SEQUENCE LISTING (12 sequences)

SEQ ID NO: 1 - DNA, 34 bp, Artificial
  Primer/probe
aacggtacctcagccatggagcagcctcaggagg

SEQ ID NO: 2 - DNA, 34 bp, Artificial
  Primer/probe
taagtcgacccgttagtacatgtccttgtagatc

SEQ ID NO: 3 - DNA, 33 bp, Artificial
  Primer/probe
ttagaattcgacatggacaccaaacatttcctg

SEQ ID NO: 4 - DNA, 33 bp, Artificial
  Primer/probe
cccctcgagctaagtcatttggtgcggcgcctc

SEQ ID NO: 5 - DNA, 36 bp, Artificial
  Primer/probe
tcgacaggggaccaggacaaaggtcacgttcgggag

SEQ ID NO: 6 - DNA, 36 bp, Artificial
  Primer/probe
tcgactcccgaacgtgacctttgtcctggtcccctg

SEQ ID NO: 7 - DNA, 28 bp, Artificial
  Primer/probe
cccagatctccccagcgtcttgtcattg

SEQ ID NO: 8 - DNA, 28 bp, Artificial Sequence
  Primer/probe
tcaccatggtcaagcttttaagcgggtc

SEQ ID NO: 9 - DNA, 33 bp, Artificial
  Primer/probe
gtgggtaccgaaatgaccatggttgacacagag

SEQ ID NO: 10 - DNA, 33 bp, Artificial
  Primer/probe
ggggtcgaccaggactctctgctagtacaagtc

SEQ ID NO: 11 - DNA, 33 bp, Artificial
  Primer/probe
cgtcgacccggcggccccatggacctgcccccg

SEQ ID NO: 12 - DNA, 33 bp, Artificial
  Primer/probe
catcgattagcagtggcgttacttctgggactt

(19) United States
(12) Patent Application Publication
(10) Pub. No.: US 2006/0148858 A1
(43) Pub. Date: Jul. 6, 2006

(54) 1, 2-Azole derivatives with hypoglycemic and hypolipidemic activity

(76) Inventors: Tsuyoshi Maekawa, Nara (JP); Ryoma Hara, Osaka (JP); Hiroyuki Odaka, Hyogo (JP); Hiroyuki Kimura, Osaka (JP); Hideya Mizufune, Osaka (JP); Kohji Fukatsu, Hyogo (JP)

Correspondence Address: TAKEDA PHARMACEUTICALS NORTH AMERICA, INC;INTELLECTUAL PROPERTY DEPARTMENT, 475 HALF DAY ROAD, SUITE 500, LINCOLNSHIRE, IL, 60069, US

(21) Appl. No.: 10/517,214

(22) PCT Filed: May 22, 2003
(86) PCT No.: PCT/JP03/06389

§ 371 Date: Mar. 1, 2005

(30) Foreign Application Priority Data
May 24, 2002 (JP) 2002-151405
Sep. 30, 2002 (JP) 2002-287161
Jan. 24, 2003 (JP) 2003-16748

(51) Int. Cl. A61K 31/4439; A61K 31/42; C07D 413/02; C07D 403/02

(52) U.S. Cl. 514/341; 514/378; 546/275.4; 548/247

(57) ABSTRACT

A compound represented by the formula (1) wherein ring A is a ring optionally having 1 to 3 substituents; ring B is a 1,2-azole ring which may further have 1 to 3 substituents; Xa, Xb and Xc are the same or different and each is a bond, —O—, —S— and the like; Ya is a divalent aliphatic hydrocarbon residue having 1 to 20 carbon atoms; Yb and Yc are the same or different and each is a bond or a divalent aliphatic hydrocarbon residue having 1 to 20 carbon atoms; ring C is a monocyclic aromatic ring which may further have 1 to 3 substituents; and R represents —OR⁴ (R⁴ is hydrogen atom or optionally substituted hydrocarbon group) and the like, or a salt thereof or a prodrug thereof is useful as an agent for the prophylaxis or treatment of diabetes and the like.

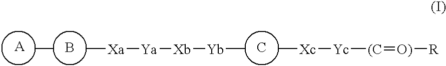

### TECHNICAL FIELD

The present invention relates to a 1,2-azole derivative having an excellent hypoglycemic action and hypolipidemic action, which is useful as an agent for the prophylaxis or treatment of diabetes, hyperlipidemia, arteriosclerosis, impaired glucose tolerance and the like.

### BACKGROUND ART

Peroxisome proliferator-activated receptor gamma (PPARγ), a member of the intranuclear hormone receptor superfamily, which is typically exemplified by steroid hormone receptors and thyroid hormone receptors, plays an important role as a master regulator in the differentiation of adipocytes with its expression induced in the very early stage of adipocyte differentiation. PPARγ forms a dimer with the retinoid X receptor (RXR) by binding to a ligand, and binds to a responsive site of the target gene in the nucleus to directly control (activate) transcription efficiency. In recent years, the possibility that 15-deoxy-Δ^{12.14} prostaglandin J₂, a metabolite of prostaglandin D₂, serves as an endogenous ligand for PPARγ, has been suggested, and it has been shown that a class of insulin sensitivity enhancers, typically exemplified by thiazolidinedione derivatives, possess ligand activity for PPARγ, and that its potency is proportional to its hypoglycemic action or adipocyte differentiation-promoting action (Cell, vol. 83, p. 803 (1995); The Journal of Biological Chemistry, vol. 270, p. 12953 (1995); Journal of Medicinal Chemistry, vol. 39, p. 655 (1996)). Furthermore, in recent years, it has been shown that 1) PPARγ is expressed in cultured cells of human liposarcoma origin, whose proliferation is ceased by the addition of a PPARγ ligand (Proceedings of the National Academy of Sciences of the United States of America, vol. 94, p. 237 (1997)), 2) nonsteroidal anti-inflammatory drugs, typically exemplified by indomethacin and fenoprofen, have PPARγ ligand activity (The Journal of Biological Chemistry, vol. 272, p. 3406 (1997)), 3) PPARγ is expressed at high levels in activated macrophages, with the transcription of a gene involved in inflammation inhibited by the addition of a ligand therefor (Nature, vol. 391, p. 79 (1998)), 4) PPARγ ligands suppress the production of inflammatory cytokines (TNFα, IL-1β, IL-6) by monocytes (Nature, vol. 391, p. 82 (1998)), 5) hypertrophy of adipocyte, accumulation of lipid and expression of insulin resistance are suppressed in PPARγ hetero deficient mouse (Molecular Cell, vol. 4, p. 597 (1999)), 6) PPARγ ligand inhibits differentiation of 10T1/2 cells to adipocytes by PPARγ agonist (Proceedings of The National Academy of Sciences of The United States of America, vol. 96, p. 6102 (1999)), 7) PPARγ ligand suppresses differentiation of 3T3-L1 cells to adipocytes by PPARγ agonist (Molecular Endocrinology, vol. 14, p. 1425 (2000)) and the like.

Peroxisome proliferator-activated receptor delta (PPARδ) is a member of the intranuclear hormone receptor PPAR family, forms a dimer with a retinoid X receptor (RXR) by ligand binding as in other PPAR families, and binds with a responsive element located upstream of the target gene in nucleus, thereby directly controlling transcription efficiency. As the ligand of PPARδ, long chain fatty acids and carbaprostacyclin can be mentioned; however, a target gene specific to PPARδ has not been identified as yet. PPARδ shows ubiquitous expression, but shows particularly strong expression in gut, kidney and heart. As regards PPARδ, it has been reported that PPARδ shows differentiation-promoting effect on mouse preadipocytes (The Journal of Biological Chemistry, vol. 274, p. 21920-21925 (1999); The Journal of Biological Chemistry, vol. 275, p. 38768-38773 (2000); The Journal of Biological Chemistry, vol. 276, p. 3175-3182 (2001)); it shows UCP-2 and UCP-3 expression-promoting effect on rat and human skeletal muscle cells (The Journal of Biological Chemistry, vol. 276, p. 10853-10860 (2001); Endocrinology, vol. 142, p. 4189-4194 (2001)); it shows differentiation-promoting effect on oligodendrocytes (Molecular Cell Biology, vol. 20, p.5119-5128 (2000); Glia, vol. 33, p. 191-204 (2001); it shows HDL-C increasing effect in db/db mouse (FEBS letters, vol. 473, p. 333-336 (2000)); it shows HDL-C increasing effect and LDL-C, VLDL and TG-lowering effect in obesity Rhesus monkey; and it shows promoting effect on cholesterol transport of human monocyte THP-1 cells via ApoA1 (Proceedings of The National Academy of Sciences of The United States of America, vol. 98, p. 5306-5311 (2001)). Moreover, it has been reported that PPARδ is involved in colon cancer (Cell, vol. 99, p. 335-345 (1999); Proceedings of The National Academy of Sciences of The United States of America, vol. 98, p. 2598-2603 (2001)), embryo implantation during gestation (Genes and Development, vol. 13, p. 1561-1574 (1999)), bone resorption in osteoclasts (The Journal of Biological Chemistry, vol. 275, p. 8126-8132 (2000)), apoptosis in inflammation (Genes and Development, vol. 15, p. 3263-3277 (2001)), and regulation of type 2 acyl-CoA synthetase in brain (The Journal of Biological Chemistry, vol. 274, p. 35881-35888 (1999)).

As PPAR ligands, the following compounds are known.
(1) As a PPAR receptor ligand, a compound represented by the formula 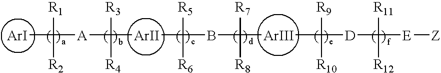
wherein 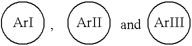
and are independently aryl and the like; A is —O— and the like; B is —O— and the like; D is —O— and the like; E is a bond or ethylene group; a, b, c and e are each 0-4; d is 0-5; f is 0-6; R₁, R₃, R₅, R₇, R₉ and R₁₁, are independently hydrogen and the like; R₂, R₄, R₆, R₈, R₁₀ and R₁₂ are independently —(CH)_{q}—X; q is 0-3; X is hydrogen and the like; Z is R₂₁O₂C— and the like; and R₂₁ is hydrogen and the like has been reported (WO00/64876).
(2) As a retinoid-related receptor function regulator, a compound represented by the formula 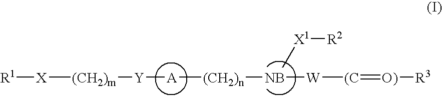
wherein R¹ is an optionally substituted hydrocarbon group or an optionally substituted heterocyclic group; X is a bond, O, S, —CO—, —CS—, —CR⁴(OR⁵)— or —NR⁶— (R⁴ and R⁶ are each a hydrogen atom or an optionally substituted hydrocarbon group, R⁵ is a hydrogen atom or a hydroxy-protecting group); m is 0-3; Y is O, S, —SO—, —SO₂—, —NR⁷—, —CONR⁷— or —NR⁷CO— (R⁷ is a hydrogen atom or an optionally substituted hydrocarbon group); ring A is an aromatic ring which may further have 1 to 3 substituents; n is 1-8; ring B is a nitrogen-containing 5-membered heterocyclic ring which may be further substituted by alkyl group; X¹ is a bond, O, S, —SO—, —SO2—, —O—SO2— or —NR¹⁶—(R¹⁶ is a hydrogen atom or an optionally substituted hydrocarbon group); R² is a hydrogen atom, an optionally substituted hydrocarbon group or an optionally substituted heterocyclic group; W is a bond or a C1-20 divalent hydrocarbon residue; and R³ is —OR⁸ (R⁸ is a hydrogen atom or an optionally substituted hydrocarbon group) or —NR⁹R¹⁰ (R⁹ and R¹⁰ are the same or different and each is a hydrogen atom, an optionally substituted hydrocarbon group, an optionally substituted heterocyclic group or an optionally substituted acyl group, or R⁹ and R¹⁰ are bonded to each other to form a ring) has been reported (WO01/38325).
(3) As a selective activator of human PPARδ, a compound represented by the formula 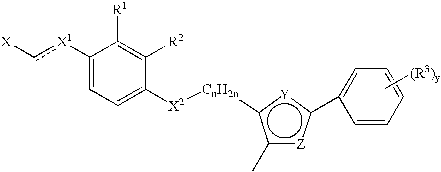
wherein X is COOH or a tetrazolyl group; X¹ is NH, NCH₃, O, S, a bond and the like; X² is O or S; R¹ and R² are independently H, CH₃, OCH₃ or a halogen; n is 1 or 2; one of Y and Z is N and the other is S or O; y is 0, 1, 2, 3, 4 or 5; and R³ is CF₃ or a halogen (WO01/00603).
(4) As a PPARδ activator, a compound represented by the formula 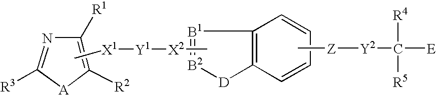
wherein A is O, S and the like; R¹, R² and R³ are each a hydrogen atom, C1-8 alkyl, C6-10 aryl group which may have substituents and the like; X¹ and X² are O, S and the like; Y¹ is a C1-8 alkylene chain which may have substituents; B¹ is CW¹ (W¹ is a hydrogen atom and the like) or N; B² is CW² (W² is a hydrogen atom and the like) or N; D is O, S and the like; Z is O or S; Y² is a C1-4 alkylene chain or a bond; R⁴ and R⁵ are each a hydrogen atom and the like; and E is a carboxyl group, a C2-8 alkoxycarbonyl group and the like, has been reported (JP-A-2001-354671).
(5) As a PPARγ agonist, a compound represented by the formula 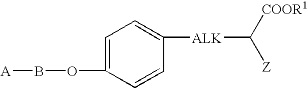
wherein A is a phenyl optionally substituted by a substituent selected from a halogen atom, C1-6 alkyl, C1-3 alkoxy, C1-3 fluoroalkoxy and the like, a 5- or 6-membered heterocyclic group containing at least one heteroatom selected from O, N and S and the like; B is C1-6 alkylene, -MC1-6 alkylene (M is O, S and the like), a 5- or 6-membered heterocyclic group containing at least one nitrogen heteroatom and at least one heteroatom selected from O, N and S, which is optionally substituted by C1-3 alkyl, Het-C1-6 alkylene (Het is a heterocyclic group) and the like; ALK is C1-3 alkylene; R¹ is a hydrogen atom or C1-3 alkyl; Z is —(C1-3 alkylene)phenyl in which phenyl may be substituted by halogen atom and the like, has been reported (WO97/31907).

In the meantime, as a 1,2-azole derivative, the following compounds are known.
(6) As a bleach accelerator releasing compound used for color photosensitive materials, the following compounds have been reported (JP-A-4-194845). 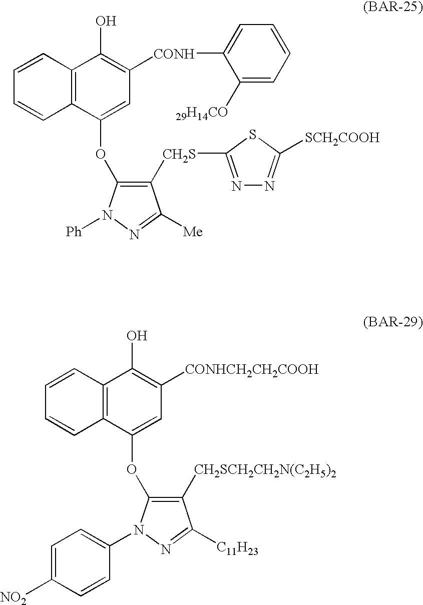
(7) As a bleach accelerator releasing compound used for color photosensitive materials, the following compounds have been reported (JP-A-4-184435). 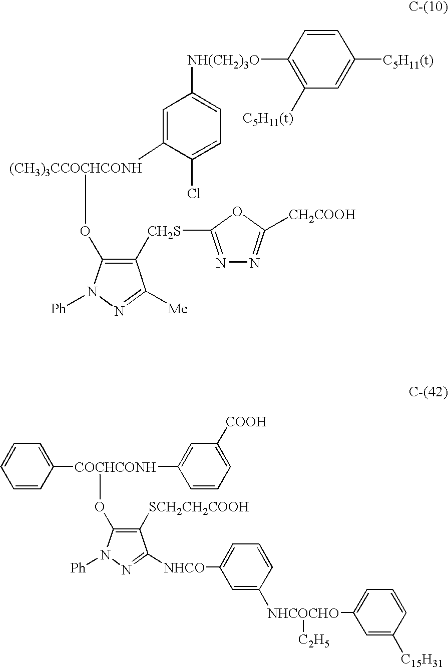
(8) As an endothelin converting enzyme inhibitor, a compound represented by the formula 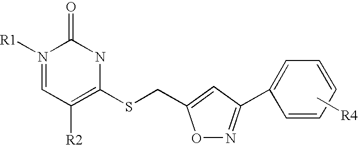
wherein R1 is C1-8 alkyl and the like which may be substituted by a substituent selected from halogen, nitro, cyano, —COOH, —COO-C1-3 alkyl and the like; R2 is C1-5 alkyl and the like; R4 is H and the like, has been reported (WO00/61579).
(9) As a platelet aggregation inhibitor, a compound represented by the formula 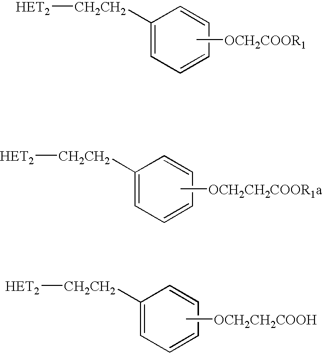
wherein R₁ is a hydrogen atom, lower alkyl or alkali metal ion; R₁ₐ is lower alkyl; HET₂ is 4,5-diphenyl-2-thiazolyl, 4,5-diphenyl-1H-imidazol-2-yl, 3,4-diphenyl-1H-pyrazol-1-yl, 4,5-diphenyl-1H-pyrazol-1-yl, 1,5-diphenyl-1H-pyrazol-3-yl and the like, has been reported (EP-A-442448).
(10) As a therapeutic agent of cardiovascular diseases, a compound represented by the formula 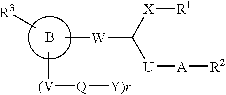
wherein B is C6-10 aryl or a heterocyclic ring containing 1 to 9 carbon atoms and up to 3 heteroatoms; r is 0 or 1; V is void or O and the like; Q is void, O or saturated or unsaturated alkylene and the like; Y is a hydrogen atom and the like; R³ is a hydrogen atom, halogen and the like; W is alkylene and the like; U is alkylene and the like; A is void or C6-10 aryl or an aromatic heterocyclic ring containing 1 to 9 carbon atoms and up to 3 heteroatoms; R² is CN, tetrazolyl, COOR²⁶ or CONR²⁷R²⁸ (R²⁶, R²⁷ and R²⁸ are each a hydrogen atom and the like); X is alkylene and the like; R1 is CN, tetrazolyl, COOR³⁵ or CONR³⁶R³⁷ (R³⁵, R³⁶ and R³⁷ are each a hydrogen atom and the like) has been reported (WO01/19778).

### DISCLOSURE OF THE INVENTION

There is a demand for development of a 1,2-azole derivative useful as an agent for the prophylaxis or treatment of diabetes, hyperlipidemia, arteriosclerosis, impaired glucose tolerance etc., and having pharmaceutically excellent properties such as low side effects, etc.

Accordingly, the present invention relates to
1) a compound represented by the formula 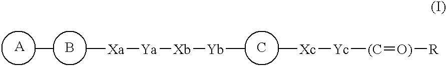
wherein
ring A is a ring optionally having 1 to 3 substituents;
ring B is a 1,2-azole ring optionally further having 1 to 3 substituents;
Xa, Xb and Xc
are the same or different and each is a bond, —O—, —S—, —SO—, —SO₂—, —CO—, —CS—, —CR¹(OR²)—, —NR³—, —CONR³— or —NR³CO— (R¹ is a hydrogen atom or an optionally substituted hydrocarbon group, R² is a hydrogen atom or a hydroxy-protecting group, and R³ is a hydrogen atom, an optionally substituted hydrocarbon group or an amino-protecting group);
Ya is a divalent aliphatic hydrocarbon residue having 1 to 20 carbon atoms;
Yb and Yc
are the same or different and each is a bond or a divalent aliphatic hydrocarbon residue having 1 to 20 carbon atoms;
ring C is a monocyclic aromatic ring optionally further having 1 to 3 substituents; and
R represents —OR⁴ (R⁴ is a hydrogen atom or an optionally substituted hydrocarbon group) or —NR⁵R⁶ (R⁵ and R⁶ are the same or different and each is a hydrogen atom, an optionally substituted hydrocarbon group or an optionally substituted heterocyclic group, or R⁵ and R⁶ form, together with the adjacent nitrogen atom, an optionally substituted heterocyclic ring), provided that,
(1) when the 1,2-azole ring represented by ring B is pyrazole, ring C is not thiadiazole or oxadiazole;
(2) when the 1,2-azole ring represented by ring B is isoxazole, ring C is not an optionally substituted pyridone; and
(3) when the 1,2-azole ring represented by ring B is pyrazole and Xa and Xb are each a bond, ring C is not a benzene ring,
   or a salt thereof,
   2) the compound of the aforementioned 1), wherein the ring represented by ring A is an aromatic ring,
   3) the compound of the aforementioned 2), wherein the aromatic ring is a benzene ring, a pyridine ring or a pyridazine ring,
   4) the compound of the aforementioned 1), wherein the 1,2-azole ring represented by ring B is pyrazole,
   5) the compound of the aforementioned 1), wherein the substituent that ring B is optionally further having is a hydrocarbon group,
   6) the compound of the aforementioned 1), wherein the substituent that ring B is optionally further having is an alkoxy group,
   7) the compound of the aforementioned 1), wherein Ya is C₁₋₆ alkylene or C₂₋₆ alkenylene,
   8) the compound of the aforementioned 1), wherein Xb is —O—, —S—, —SO—, —SO₂—, —CO—, —CS—, —CR¹(OR²)—, —NR³—-CONR³— or —NR³CO— (R¹ is a hydrogen atom or an optionally substituted hydrocarbon group, R² is a hydrogen atom or a hydroxy-protecting group, and R³ is a hydrogen atom, an optionally substituted hydrocarbon group or an amino-protecting group),
   9) the compound of the aforementioned 1), wherein the monocyclic aromatic ring represented by ring C is a benzene ring,
   10) the compound of the aforementioned 1), wherein the monocyclic aromatic ring represented by ring C is pyrazole,
   11) the compound of the aforementioned 1), wherein R represents —OR⁴ (R⁴ is a hydrogen atom or an optionally substituted hydrocarbon group),
   12) the compound of the aforementioned 1), wherein Xa is a bond,
   13) the compound of the aforementioned 1), wherein Xb is —O—,
   14) the compound of the aforementioned 1), wherein Yb is a bond,
   15) the compound of the aforementioned 1), wherein Xc is a bond or —O—,
   16) the compound of the aforementioned 1), wherein Yc is C₁₋₆ alkylene or C₂₋₆ alkenylene,
   17) the compound of the aforementioned 1), which is 3-[1-phenyl-3-(4-{3-[4-(trifluoromethyl)phenyl]-5-isoxazolyl}butoxy)-1H-pyrazol-5-yl]propionic acid;
2-[3-(3-{3-ethoxy-1-[5-(trifluoromethyl)-2-pyridyl]-1H-pyrazol-4-yl}propoxy)phenoxy]-2-methylpropionic acid;
3-[2-ethoxy-4-(3-{3-ethoxy-1-[5-(trifluoromethyl)-2-pyridyl]-1H-pyrazol-4-yl}propoxy)phenyl]propionic acid;
3-[3-(3-{3-ethoxy-1-[5-(trifluoromethyl)-2-pyridyl]-1H-pyrazol-4-yl}propoxy)-1-phenyl-1H-pyrazol-5-yl]propionic acid;
[1-phenyl-3-(4-{3-propyl-1-[5-(trifluoromethyl)-2-pyridinyl]-1H-pyrazol-4-yl}butoxy)-1H-pyrazol-4-yl]acetic acid;
[2-(3-{3-isopropyl-1-[5-(trifluoromethyl)-2-pyridyl]-1H-pyrazol-4-yl}propoxy)-3-methoxyphenyl]acetic acid;
[2-(3-{3-(1-ethylpropyl)-1-[5-(trifluoromethyl)-2-pyridyl]-1H-pyrazol-4-yl}propoxy)-3-methoxyphenyl]acetic acid;
(2-{3-[1-(5-chloro-2-pyridyl)-3-(1-ethylpropyl)-1H-pyrazol-4-yl]propoxy}-3-methoxyphenyl)acetic acid;
[3-ethyl-2-(3-{3-isopropyl-1-[6-(trifluoromethyl)pyridazin-3-yl]-1H-pyrazol-4-yl}propoxy)phenyl]acetic acid;
[2-(3-{3-isopropyl-1-[6-(trifluoromethyl)pyridazin-3-yl]-1H-pyrazol-4-yl}propoxy)-3-methoxyphenyl]acetic acid;
[3-(3-{3-isopropyl-1-[5-(trifluoromethyl)-2-pyridinyl]-1H-pyrazol-4-yl}propoxy)-1-methyl-1H-pyrazol-4-yl]acetic acid;
[1-ethyl-5-(3-{3-isopropyl-1-[5-(trifluoromethyl)-2-pyridinyl]-1H-pyrazol-4-yl}propoxy)-1H-pyrazol-4-yl]acetic acid;
[1-ethyl-5-(3-{3-propyl-1-[5-(trifluoromethyl)-2-pyridinyl]-1H-pyrazol-4-yl}propoxy)-1H-pyrazol-4-yl]acetic acid;
(2-{3-[1-(5-bromo-2-pyridinyl)-3-(1-ethylpropyl)-1H-pyrazol-4-yl]propoxy}-3-methoxyphenyl)acetic acid; or
[2-(3-{3-tert-butyl-1-[6-(trifluoromethyl)pyridazin-3-yl]-1H-pyrazol-4-yl}propoxy)-3-methylphenyl]acetic acid.
   18) a prodrug of the compound of the aforementioned 1) or a salt thereof,
   19) a pharmaceutical composition comprising the compound of the aforementioned 1) or a salt thereof or a prodrug thereof,
   20) an agent for the prophylaxis or treatment of diabetes, which comprises a compound represented by the formula 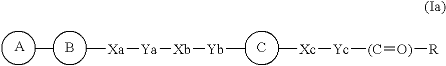
   wherein
ring A is a ring optionally having 1 to 3 substituents;
ring B is a 1,2-azole ring optionally further having 1 to 3 substituents;
Xa, Xb and Xc
are the same or different and each is a bond, —O—, —S—, —SO—, —SO₂—, —CO—, —CS—, —CR¹(OR²)—, —NR³—, —CONR³— or —NR³CO— (R¹ is a hydrogen atom or an optionally substituted hydrocarbon group, R² is a hydrogen atom or a hydroxy-protecting group, and R³ is a hydrogen atom, an optionally substituted hydrocarbon group or an amino-protecting group);
Ya is a divalent aliphatic hydrocarbon residue having 1 to 20 carbon atoms;
Yb and Yc
are the same or different and each is a bond or a divalent aliphatic hydrocarbon residue having 1 to 20 carbon atoms;
ring C is a monocyclic aromatic ring optionally further having 1 to 3 substituents; and
R represents —OR⁴ (R⁴ is a hydrogen atom or an optionally substituted hydrocarbon group) or —NR⁵R⁶ (R⁵ and R⁶ are the same or different and each is a hydrogen atom, an optionally substituted hydrocarbon group or an optionally substituted heterocyclic group, or R⁵ and R⁶ form, together with the adjacent nitrogen atom, an optionally substituted heterocyclic ring),
   or a salt thereof or a prodrug thereof,
   21) an agent for the prophylaxis or treatment of hyperlipidemia, which comprises a compound represented by the formula (Ia) or a salt thereof or a prodrug thereof,
   22) an agent for the prophylaxis or treatment of arteriosclerosis, which comprises a compound represented by the formula 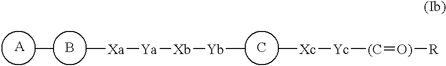
   wherein
ring A is a ring optionally having 1 to 3 substituents;
ring B is a 1,2-azole ring optionally further having 1 to 3 substituents;
Xa, Xb and Xc
are the same or different and each is a bond, —O—, —S—, —SO—, —SO₂—, —CO—, —CS—, —CR¹(OR²)—, —NR³—, —CONR³— or —NR³CO— (R¹ is a hydrogen atom or an optionally substituted hydrocarbon group, R² is a hydrogen atom or a hydroxy-protecting group, and R³ is a hydrogen atom, an optionally substituted hydrocarbon group or an amino-protecting group);
Ya is a divalent aliphatic hydrocarbon residue having 1 to 20 carbon atoms;
Yb and Yc
are the same or different and each is a bond or a divalent aliphatic hydrocarbon residue having 1 to 20 carbon atoms;
ring C is a monocyclic aromatic ring optionally further having 1 to 3 substituents; and
R represents —OR⁴ (R⁴ is a hydrogen atom or an optionally substituted hydrocarbon group) or —NR⁵R⁶ (R⁵ and R⁶ are the same or different and each is a hydrogen atom, an optionally substituted hydrocarbon group or an optionally substituted heterocyclic group, or R⁵ and R⁶ form, together with the adjacent nitrogen atom, an optionally substituted heterocyclic ring),
provided that, when the 1,2-azole ring represented by ring B is isoxazole, ring C is not an optionally substituted pyridone,
   or a salt thereof or a prodrug thereof.
   23) an agent for the prophylaxis or treatment of impaired glucose tolerance, which comprises a compound represented by the formula (Ia) or a salt thereof or a prodrug thereof,
   24) a retinoid-related receptor function regulating agent, which comprises a compound represented by the formula (Ia) or a salt thereof or a prodrug thereof,
   25) the agent of the aforementioned 24), which is a peroxisome proliferator-activated receptor ligand,
   26) the agent of the aforementioned 24), which is a retinoid X receptor ligand,
   27) an insulin resistance improving agent, which comprises a compound represented by the formula (Ia) or a salt thereof or a prodrug thereof,
   28) a method for the prophylaxis or treatment of diabetes in a mammal in need thereof, which comprises administering to the mammal a compound represented by the formula (Ia) or a salt thereof or a prodrug thereof,
   29) use of a compound represented by the formula (Ia) or a salt thereof or a prodrug thereof, for the production of an agent for the prophylaxis or treatment of diabetes,
   30) a GPR40 receptor function modulator comprising a compound represented by the formula (Ia) or a salt thereof or a prodrug thereof,
   31) a production method of a compound represented by the formula 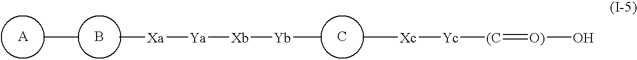
   wherein the symbols in the formula are as defined in the aforementioned 1), or a salt thereof, which comprises subjecting a compound represented by the formula 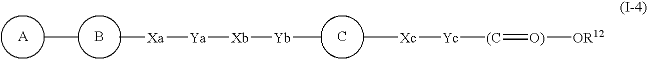
   wherein R¹² is an optionally substituted hydrocarbon group and other symbols are as defined above, or a salt thereof to a hydrolysis reaction,
   32) a production method of a compound represented by the formula 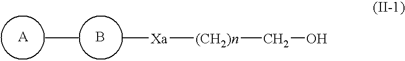
   wherein n is an integer of 0 to 5 and other symbols are as defined in the aforementioned 1), or a salt thereof, which comprises subjecting a compound represented by the formula 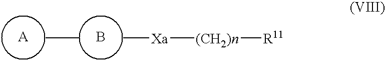
   wherein R¹¹ is CHO or COOR¹³ (R¹³ is an alkyl group having 1-6 carbon atoms), and other symbols are as defined above, or a salt thereof to a reduction reaction,
   33) a compound represented by the formula 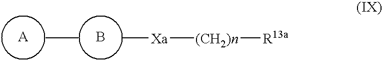
   wherein n is an integer of 0 to 5, R^{13a} is CH₂OH, CHO or COOR¹⁴ (R¹⁴ is an alkyl group having 1-6 carbon atoms), and other symbols are as defined in the aforementioned 1), or a salt thereof, and the like.

The definition of each symbol in the formulas (I), (Ia) and (Ib) is explained in detail in the following.

As the ring represented by ring A, for example, aromatic rings such as aromatic hydrocarbon, aromatic heterocyclic ring and the like; and non-aromatic rings such as alicyclic hydrocarbon, non-aromatic heterocyclic ring and the like can be mentioned.

As the aromatic hydrocarbon, for example, aromatic hydrocarbon having 6 to 14 carbon atoms can be mentioned. As preferable examples of the aromatic hydrocarbon, benzene, naphthalene, anthracene, phenanthrene, acenaphthylene, indene and the like can be mentioned. Of these, benzene, naphthalene and the like are preferable.

As the aromatic heterocyclic ring, for example, a 5- to 7-membered monocyclic aromatic heterocyclic ring, which contains, besides carbon atom, 1 to 4 heteroatoms selected from oxygen atom, sulfur atom and nitrogen atom as ring-constituting atom, or condensed aromatic heterocyclic ring can be mentioned. As the condensed aromatic heterocyclic ring, for example, a ring wherein the above-mentioned. 5- to 7-membered monocyclic aromatic heterocyclic ring and a 6-membered ring containing 1 or 2 nitrogen atoms, a benzene ring or a 5-membered ring containing one sulfur atom are condensed, and the like can be mentioned.

Preferable examples of the aromatic heterocyclic ring include furan, thiophene, pyridine, pyrimidine, pyridazine, pyrazine, pyrrole, imidazole, pyrazole, isoxazole, isothiazole, oxazole, thiazole, oxadiazole, thiadiazole, triazole, tetrazole, quinoline, quinazoline, quinoxaline, benzofuran, benzothiophene, benzoxazole, benzothiazole, benzimidazole, indole, 1H-indazole, 1H-pyrrolo[2,3-b]pyrazine, 1H-pyrrolopyridine, 1H-imidazopyridine, 1H-imidazopyrazine, triazine, isoquinoline, benzothiadiazole and the like.

The aromatic heterocyclic ring is preferably a 5- or 6-membered aromatic heterocyclic ring, more preferably furan, thiophene, pyridine, pyrimidine, pyrazole, oxazole, thiazole, pyridazine, oxadiazole, thiadiazole and the like.

As the alicyclic hydrocarbon, a saturated or unsaturated alicyclic hydrocarbon having 3 to 12 carbon atoms, for example, cycloalkane, cycloalkene, cycloalkadiene and the like can be mentioned.

Preferable examples of cycloalkane include cycloalkane having 3 to 10 carbon atoms such as cyclopropane, cyclobutane, cyclopentane, cyclohexane, cycloheptane, cyclooctane, bicyclo[2.2.1]heptane, bicyclo[2.2.2]octane, bicyclo[3.2.1]octane, bicyclo[3.2.2]nonane, bicyclo[3.3.1]nonane, bicyclo[4.2.1]nonane, bicyclo[4.3.1]decane and the like.

Preferable examples of cycloalkene include cycloalkene having 3 to 10 carbon atoms, such as cyclopentene, cyclohexene and the like.

Preferable examples of cycloalkadiene include cycloalkadiene having 4 to 10 carbon atoms, such as 2,4-cyclopentadiene, 2,4-cyclohexadiene, 2,5-cyclohexadiene and the like.

As the non-aromatic heterocyclic ring, for example, a 5- to 7-membered monocyclic non-aromatic heterocyclic ring, which contains, besides carbon atom, 1 to 4 heteroatoms selected from oxygen atom, sulfur atom and nitrogen atom as ring-constituting atom, or condensed non-aromatic heterocyclic ring can be mentioned. As the condensed non-aromatic heterocyclic ring, for example, a ring wherein the above-mentioned 5- to 7-membered monocyclic non-aromatic heterocyclic ring and a 6-membered ring containing 1 or 2 nitrogen atoms, a benzene ring or a 5-membered ring containing one sulfur atom are condensed, and the like can be mentioned.

Preferable examples of the non-aromatic heterocyclic ring include pyrrolidine, pyrroline, pyrazolidine, piperidine, piperazine, morpholine, thiomorpholine, hexamethyleneimine, oxazolidine, thiazolidine, imidazolidine, imidazoline, tetrahydrofuran, azepane, tetrahydropyridine and the like.

The ring represented by ring A is preferably an aromatic ring such as aromatic hydrocarbon, aromatic heterocyclic ring and the like, more preferably an aromatic hydrocarbon having 6 to 14 carbon atoms or a 5- or 6-membered aromatic heterocyclic ring. Of these, benzene, pyridine, pyrimidine, pyridazine, oxadiazole, thiadiazole and the like are preferable. Especially, benzene, pyridine, pyridazine and the like are preferable. The ring represented by ring A is most preferably pyridine or pyridazine.

The ring represented by ring A may have 1 to 3 substituents at substitutable positions. As the substituent, for example, “halogen atom”, “nitro group”, “cyano group”, “optionally substituted aliphatic hydrocarbon group”, “optionally substituted alicyclic hydrocarbon group”, “optionally substituted aromatic hydrocarbon group”, “optionally substituted aromatic aliphatic hydrocarbon group”, “optionally substituted heterocyclic group”, “optionally substituted acyl group”, “optionally substituted amino group”, “optionally substituted hydroxy group”, “optionally substituted thiol group”, “optionally esterified or amidated carboxyl group” and the like can be mentioned.

As the “halogen atom”, fluorine, chlorine, bromine and iodine can be mentioned. Of these, fluorine and chlorine are preferable.

As the aliphatic hydrocarbon group of the “optionally substituted aliphatic hydrocarbon group”, a straight-chain or branched aliphatic hydrocarbon group having 1 to 15 carbon atoms are preferable. As the aliphatic hydrocarbon group, for example, alkyl group, alkenyl group, alkynyl group and the like can be mentioned.

Preferable examples of alkyl group include alkyl group having 1 to 10 carbon atoms, such as methyl, ethyl, propyl, isopropyl, butyl, isobutyl, sec.-butyl, t.-butyl, pentyl, isopentyl, neopentyl, 1-ethylpropyl, hexyl, isohexyl, 1,1-dimethylbutyl, 2,2-dimethylbutyl, 3,3-dimethylbutyl, 2-ethylbutyl, heptyl, octyl, nonyl, decyl, 1-methylbutyl and the like.

Preferable examples of alkenyl group include alkenyl group having 2 to 10 carbon atoms such as ethenyl, 1-propenyl, 2-propenyl, 2-methyl-1-propenyl, 1-butenyl, 2-butenyl, 3-butenyl, 3-methyl-2-butenyl, 1-pentenyl, 2-pentenyl, 3-pentenyl, 4-pentenyl, 4-methyl-3-pentenyl, 1-hexenyl, 3-hexenyl, 5-hexenyl, 1-heptenyl, 1-octenyl and the like.

Preferable examples of alkynyl group include alkynyl group having 2 to 10 carbon atoms, such as ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl, 1-heptynyl, 1-octynyl and the like.

As the substituent of the “optionally substituted aliphatic hydrocarbon group”, for example, halogen atom (e.g., fluorine, chlorine, bromine, iodine); sulfo group; cyano group; azido group; nitro group; nitroso group; cycloalkyl group having 3 to 10 carbon atoms; aromatic heterocyclic group (e.g., thienyl, furyl, pyridyl, oxazolyl, thiazolyl); non-aromatic heterocyclic group (e.g., tetrahydrofuryl, morpholino, thiomorpholino, piperidino, pyrrolidinyl, piperazinyl); amino group which may be mono- or di-substituted by a substituent selected from alkyl group having 1 to 4 carbon atoms and acyl group having 2 to 8 carbon atoms (e.g., alkanoyl group); amidino group; acyl group having 2 to 8 carbon atoms (e.g., alkanoyl group); carbamoyl group which may be mono- or di-substituted by alkyl group having 1 to 4 carbon atoms; sulfamoyl group which may be mono- or di-substituted by alkyl group having 1 to 4 carbon atoms; carboxyl group; alkoxycarbonyl group having 2 to 8 carbon atoms; hydroxy group; alkoxy group having 1 to 6 carbon atoms which may be substituted by 1 to 3 halogen atoms (e.g., fluorine, chlorine, bromine, iodine); aralkyloxy group having 7 to 13 carbon atoms; aryloxy group having 6 to 14 carbon atoms (e.g., phenyloxy, naphthyloxy); thiol group; alkylthio group having 1 to 6 carbon atoms which may be substituted by 1 to 3 halogen atoms (e.g., fluorine, chlorine, bromine, iodine); aralkylthio group having 7 to 13 carbon atoms; arylthio group having 6 to 14 carbon atoms (e.g., phenylthio, naphthylthio) and the like can be mentioned. The number of substituent is, for example, 1 to 3.

As the alicyclic hydrocarbon group of the “optionally substituted alicyclic hydrocarbon group”, saturated or unsaturated alicyclic hydrocarbon group having 3 to 10 carbon atoms is preferable. As the alicyclic hydrocarbon group, for example, cycloalkyl group, cycloalkenyl group, cycloalkadienyl group and the like can be mentioned.

Preferable examples of the cycloalkyl group include cycloalkyl group having 3 to 10 carbon atoms, such as cyclopropyl, cyclobutyl, cyclopentyl, cyclohexyl, cycloheptyl and the like.

Preferable examples of the cycloalkenyl group include cycloalkenyl group having 3 to 10 carbon atoms, such as 1-cyclopentenyl, 2-cyclopentenyl, 3-cyclopentenyl, 1-cyclohexenyl, 2-cyclohexenyl, 3-cyclohexenyl, 1-cycloheptenyl, 2-cycloheptenyl, 3-cycloheptenyl and the like.

Preferable examples of the cycloalkadienyl group include cycloalkadienyl group having 5 to 10 carbon atoms, such as 2,4-cycloheptadienyl and the like.

As the aromatic hydrocarbon group of the “optionally substituted aromatic hydrocarbon group”, aryl group having 6 to 14 carbon atoms is preferable. As the aryl group, for example, phenyl, naphthyl, anthryl, phenanthryl, acenaphthylenyl and the like can be mentioned. Of these, phenyl, 1-naphthyl, 2-naphthyl and the like are preferable.

As the aromatic aliphatic hydrocarbon group of the “optionally substituted aromatic aliphatic hydrocarbon group”, aromatic aliphatic hydrocarbon group having 7 to 13 carbon atoms is preferable. As the aromatic aliphatic hydrocarbon group, for example, aralkyl group, arylalkenyl group and the like can be mentioned.

Preferable examples of the aralkyl group include aralkyl group having 7 to 13 carbon atoms, such as benzyl, phenethyl, naphthylmethyl, benzhydryl and the like.

Preferable examples of the arylalkenyl group include arylalkenyl group having 8 to 13 carbon atoms, such as styryl and the like.

As the heterocyclic group of the “optionally substituted heterocyclic group”, for example, a 5- to 7-membered monocyclic heterocyclic group, which contains, besides carbon atom, 1 to 4 heteroatoms selected from oxygen atom, sulfur atom and nitrogen atom as ring-constituting atom, or condensed heterocyclic group can be mentioned. As the condensed heterocyclic group, for example, a group wherein the above-mentioned 5- to 7-membered monocyclic heterocyclic group is condensed with a 6-membered ring containing 1 or 2 nitrogen atoms, a benzene ring or a 5-membered ring containing one sulfur atom and the like can be mentioned.

Specific examples of the heterocyclic group include aromatic heterocyclic groups such as furyl (2-furyl, 3-furyl), thienyl (2-thienyl, 3-thienyl), pyrrolyl (1-pyrrolyl, 2-pyrrolyl, 3-pyrrolyl), imidazolyl (1-imidazolyl, 2-imidazolyl, 4-imidazolyl, 5-imidazolyl), pyrazolyl (1-pyrazolyl, 3-pyrazolyl, 4-pyrazolyl), isoxazolyl (3-isoxazolyl, 4-isoxazolyl, 5-isoxazolyl), isothiazolyl (3-isothiazolyl, 4-isothiazolyl, 5-isothiazolyl), thiazolyl (2-thiazolyl, 4-thiazolyl, 5-thiazolyl), oxazolyl (2-oxazolyl, 4-oxazolyl, 5-oxazolyl), oxadiazolyl (1,2,4-oxadiazol-3-yl, 1,2,4-oxadiazol-5-yl, 1,3,4-oxadiazol-2-yl), thiadiazolyl (1,3,4-thiadiazol-2-yl), triazolyl (1,2,4-triazol-1-yl, 1,2,4-triazol-3-yl, 1,2,3-triazol-1-yl, 1,2,3-triazol-2-yl, 1,2,3-triazol-4-yl), tetrazolyl (tetrazol-1-yl, tetrazol-5-yl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyridazinyl (3-pyridazinyl, 4-pyridazinyl), pyrazinyl (2-pyrazinyl), quinolyl (2-quinolyl, 3-quinolyl, 4-quinolyl), quinazolyl (2-quinazolyl, 4-quinazolyl), quinoxalyl (2-quinoxalyl), benzoxazolyl (2-benzoxazolyl), benzothiazolyl (2-benzothiazolyl), benzimidazolyl (benzimidazol-1-yl, benzimidazol-2-yl), indolyl (indol-1-yl, indol-3-yl), indazolyl (1H-indazol-3-yl), pyrrolopyrazinyl (1H-pyrrolo[2,3-b]pyrazin-2-yl), pyrrolopyridinyl (1H-pyrrolo[2,3-b]pyridin-6-yl), imidazopyridinyl (1H-imidazo[4,5-b]pyridin-2-yl, 1H-imidazo[4,5-c]pyridin-2-yl), imidazopyrazinyl (1H-imidazo[4,5-b]pyrazin-2-yl), benzotriazolyl (benzotriazol-1-yl) and the like; non-aromatic heterocyclic groups such as pyrrolidinyl (1-pyrrolidinyl, 2-pyrrolidinyl, 3-pyrrolidinyl), imidazolidinyl (2-imidazolidinyl, 4-imidazolidinyl), pyrazolidinyl (2-pyrazolidinyl, 3-pyrazolidinyl, 4-pyrazolidinyl), thiazolidinyl (thiazolidin-3-yl), oxazolidinyl (oxazolidin-3-yl), piperidino, morpholino, thiomorpholino, piperazinyl (1-piperazinyl), hexamethyleneiminyl (hexamethyleneimin-1-yl) and the like.

As the substituent of the aforementioned “optionally substituted alicyclic hydrocarbon group”, “optionally substituted aromatic hydrocarbon group”, “optionally substituted aromatic aliphatic hydrocarbon group” and “optionally substituted heterocyclic group”, for example, halogen atom (e.g., fluorine, chlorine, bromine, iodine); sulfo group; cyano group; azido group; nitro group; nitroso group; alkyl group having 1 to 6 carbon atoms which may be substituted by 1 to 3 halogen atoms (e.g., fluorine, chlorine, bromine, iodine); alkenyl group having 2 to 6 carbon atoms which may be substituted by 1 to 3 halogen atoms (e.g., fluorine, chlorine, bromine, iodine); cycloalkyl group having 3 to 10 carbon atoms; aryl group having 6 to 14 carbon atoms (e.g., phenyl, naphthyl); aromatic heterocyclic group (e.g., thienyl, furyl, pyridyl, oxazolyl, thiazolyl); non-aromatic heterocyclic group (e.g., tetrahydrofuryl, morpholino, thiomorpholino, piperidino, pyrrolidinyl, piperazinyl); aralkyl group having 7 to 13 carbon atoms; amino group which may be mono- or di-substituted by a substituent selected from alkyl group having 1 to 4 carbon atoms and acyl group having 2 to 8 carbon atoms (e.g., alkanoyl group); amidino group; acyl group having 2 to 8 carbon atoms (e.g., alkanoyl group); carbamoyl group which may be mono- or di-substituted by alkyl group having 1 to 4 carbon atoms; sulfamoyl group which may be mono- or di-substituted by alkyl group having 1 to 4 carbon atoms; carboxyl group; alkoxycarbonyl group having 2 to 8 carbon atoms; hydroxy group; alkoxy group having 1 to 6 carbon atoms which may be substituted by 1 to 3 halogen atoms (e.g., fluorine, chlorine, bromine, iodine); aralkyloxy group having 7 to 13 carbon atoms; aryloxy group having 6 to 14 carbon atoms (e.g., phenyloxy, naphthyloxy); thiol group; alkylthio group having 1 to 6 carbon atoms which may be substituted by 1 to 3 halogen atoms (e.g., fluorine, chlorine, bromine, iodine); aralkylthio group having 7 to 13 carbon atoms; arylthio group having 6 to 14 carbon atoms (e.g., phenylthio, naphthylthio) and the like can be mentioned. The number of substituent is, for example, 1 to 3.

The acyl group of the “optionally substituted acyl group” is exemplified by an acyl group having 1 to 13 carbon atoms, which is specifically formyl, a group represented by the formula: —COR⁷, —SO₂R⁷, —SOR⁷ or —PO₃R⁷R⁸ [wherein R⁷ and R⁸ are the same or different and each is hydrocarbon group or heterocyclic group, or R⁷ and R⁸ may form a heterocyclic ring together with the adjacent oxo-substituted phosphorus atom and two oxygen atoms] and the like.

As the hydrocarbon group represented by R⁷ or R⁸, for example, aliphatic hydrocarbon group, alicyclic hydrocarbon group, aromatic hydrocarbon group, aromatic aliphatic hydrocarbon group and the like can be mentioned.

As these aliphatic hydrocarbon group, alicyclic hydrocarbon group, aromatic hydrocarbon group and aromatic aliphatic hydrocarbon group, those exemplified as the substituent for ring A can be mentioned.

The hydrocarbon group is preferably alkyl group having 1 to 10 carbon atoms, alkenyl group having 2 to 10 carbon atoms, cycloalkyl group having 3 to 10 carbon atoms, cycloalkenyl group having 3 to 10 carbon atoms, aryl group having 6 to 14 carbon atoms, aralkyl group having 7 to 13 carbon atoms and the like.

As the heterocyclic group represented by R⁷ or R⁸, those exemplified as the substituent for ring A can be mentioned. The heterocyclic group is preferably thienyl, furyl, pyridyl and the like.

As the heterocyclic ring formed by R⁷ and R⁸ together with the adjacent oxo-substituted phosphorus atom and two oxygen atoms, for example, a 4- to 7-membered heterocyclic ring, which contains, besides carbon atom, oxo-substituted phosphorus atom and two oxygen atoms and optionally 1 or 2 heteroatoms selected from oxygen atom, nitrogen atom and sulfur atom as ring-constituting atom and the like can be mentioned. Specific examples of the heterocyclic ring include 2-oxide-1,3,2-dioxaphosphinane, 2-oxide-1,3,2-dioxaphospholane and the like.

Preferable examples of the acyl group include an alkanoyl group having 2 to 10 carbon atoms (e.g., acetyl, propionyl, butyryl, isobutyryl, valeryl, isovaleryl, pivaloyl, hexanoyl, heptanoyl, octanoyl), an alkenoyl group having 3 to 10 carbon atoms (e.g., crotonyl), a cycloalkanoyl group having 4 to 10 carbon atoms (e.g., cyclobutanecarbonyl, cyclopentanecarbonyl, cyclohexanecarbonyl, cycloheptanecarbonyl), a cycloalkenoyl group having 4 to 10 carbon atoms (e.g., 2-cyclohexenecarbonyl), an arylcarbonyl group having 7 to 13 carbon atoms (e.g., benzoyl), an aromatic heterocyclic carbonyl group (e.g., nicotinoyl, isonicotinoyl), alkylsulfinyl group having 1 to 10 carbon atoms (e.g., methylsulfinyl, ethylsulfinyl), an alkylsulfonyl group having 1 to 10 carbon atoms (e.g., methylsulfonyl, ethylsulfonyl), a (mono- or di-alkyl having 1 to 10 carbon atoms)phosphono group optionally forming a ring (e.g., dimethylphosphono, diethylphosphono, diisopropylphosphono, dibutylphosphono, 2-oxide-1,3,2-dioxaphosphinanyl) and the like.

The acyl group may have 1 to 3 substituents at substitutable positions, and as such substituent, for example, a C₁₋₆ alkyl group (e.g., methyl, ethyl) which may be substituted by 1 to 3 halogen atoms (e.g., fluorine, chlorine, iodine), a C₁₋₆ alkoxy group (e.g., methoxy, ethoxy) which may be substituted by 1 to 3 halogen atoms (e.g., fluorine, chlorine, bromine, iodine), a halogen atom (e.g., fluorine, chlorine, bromine, iodine), a nitro group, a hydroxy group, an amino group and the like can be mentioned.

As the “optionally substituted amino group”, an amino group which may be mono- or di-substituted by a substituent selected from, for example, an alkyl group having 1 to 10 carbon atoms, an alkenyl group having 2 to 10 carbon atoms, a cycloalkyl group having 3 to 10 carbon atoms, a cycloalkenyl group having 3 to 10 carbon atoms, an aryl group having 6 to 14 carbon atoms, an aralkyl group having 7 to 13 carbon atoms and an acyl group having 1 to 13 carbon atoms can be mentioned.

As these alkyl group having 1 to 10 carbon atoms, alkenyl group having 2 to 10 carbon atoms, cycloalkyl group having 3 to 10 carbon atoms, cycloalkenyl group having 3 to 10 carbon atoms, aryl group having 6 to 14 carbon atoms, aralkyl group having 7 to 13 carbon atoms and acyl group having 1 to 13 carbon atoms, those exemplified as the substituent for ring A can be mentioned.

Preferable examples of the substituted amino group include mono- or di-C₁₋₁₀ alkylamino (e.g., methylamino, dimethylamino, ethylamino, diethylamino, ethylmethylamino, propylamino, dibutylamino), mono- or di-C₂₋₁₀ alkenylamino (e.g., diallylamino), mono- or di-C₃₋₁₀ cycloalkylamino (e.g., cyclohexylamino), mono- or di-C₂₋₁₀ alkanoylamino (e.g., acetylamino, propionylamino, butyrylamino, isobutyrylamino), arylcarbonylamino group having 7 to 13 carbon atoms (e.g., benzoylamino), arylamino having 6 to 14 carbon atoms (e.g., phenylamino), N-C₁₋₁₀ alkyl-N—C₆₋₁₄ arylamino (e.g., N-methyl-N-phenylamino), C₁₋₁₀ alkylsulfonylamino (e.g., methylsulfonylamino) and the like.

As the “optionally substituted hydroxy group”, for example, a hydroxy group which may be substituted by an “alkyl group having 1 to 10 carbon atoms”, “alkenyl group having 2 to 10 carbon atoms”, “cycloalkyl group having 3 to 10 carbon atoms”, “cycloalkenyl group having 3 to 10 carbon atoms”, “aryl group having 6 to 14 carbon atoms”, “aralkyl group having 7 to 13 carbon atoms” or “acyl group having 1 to 13 carbon atoms”, each of which may be substituted, can be mentioned.

As these “alkyl group having 1 to 10 carbon atoms”, “alkenyl group having 2 to 10 carbon atoms”, “cycloalkyl group having 3 to 10 carbon atoms”, “cycloalkenyl group having 3 to 10 carbon atoms”, “aryl group having 6 to 14 carbon atoms”, “aralkyl group having 7 to 13 carbon atoms” and “acyl group having 1 to 13 carbon atoms”, those exemplified as the substituent for ring A can be mentioned.

These “alkyl group having 1 to 10 carbon atoms”, “alkenyl group having 2 to 10 carbon atoms”, “cycloalkyl group having 3 to 10 carbon atoms”, “cycloalkenyl group having 3 to 10 carbon atoms”, “aryl group having 6 to 14 carbon atoms”, “aralkyl group having 7 to 13 carbon atoms” and “acyl group having 1 to 13 carbon atoms” may have 1 to 3 substituents at substitutable positions. As such substituents, for example, a halogen atom (e.g., fluorine, chlorine, bromine, iodine), a C₁₋₆ alkoxy group (e.g., methoxy, ethoxy) which may be substituted by 1 to 3 halogen atoms (e.g., fluorine, chlorine, bromine, iodine), a hydroxy group, a nitro group, an amino group and the like can be mentioned.

As the substituted hydroxy group, for example, an alkoxy group, an alkenyloxy group, a cycloalkyloxy group, a cycloalkenyloxy group, an aryloxy group, an aralkyloxy group, an acyloxy group and the like, each of which may be substituted, can be mentioned.

Preferable examples of the alkoxy group include an alkoxy group having 1 to 10 carbon atoms, such as methoxy, ethoxy, propoxy, isopropoxy, butoxy, isobutoxy, sec.-butoxy, t.-butoxy, pentyloxy, isopentyloxy, neopentyloxy, hexyloxy, heptyloxy, nonyloxy and the like.

Preferable examples of the alkenyloxy group include an alkenyloxy group having 2 to 10 carbon atoms, such as allyloxy, crotyloxy, 2-pentenyloxy, 3-hexenyloxy and the like.

Preferable examples of the cycloalkyloxy group include a cycloalkyloxy group having 3 to 10 carbon atoms, such as cyclobutoxy, cyclopentyloxy, cyclohexyloxy and the like.

Preferable examples of the cycloalkenyloxy group include a cycloalkenyloxy group having 3 to 10 carbon atoms, such as 2-cyclopentenyloxy, 2-cyclohexenyloxy and the like.

Preferable examples of the aryloxy group include an aryloxy group having 6 to 14 carbon atoms, such as phenoxy, naphthyloxy and the like.

Preferable examples of the aralkyloxy group include an aralkyloxy group having 7 to 13 carbon atoms, such as benzyloxy, phenethyloxy, naphthylmethyloxy and the like.

Preferable examples of the acyloxy group include an acyloxy group having 2 to 13 carbon atoms, such as an alkanoyloxy having 2 to 4 carbon atoms (e.g., acetyloxy, propionyloxy, butyryloxy, isobutyryloxy) and the like.

The above-mentioned alkoxy group, alkenyloxy group, cycloalkyloxy group, cycloalkenyloxy group, aryloxy group, aralkyloxy group and acyloxy group may have 1 to 3 substituents at substitutable positions. Examples of such substituent include a halogen atom (e.g., fluorine, chlorine, bromine, iodine), a C₁₋₆ alkoxy group (e.g., methoxy, ethoxy) which may be substituted by 1 to 3 halogen atoms (e.g., fluorine, chlorine, bromine, iodine), a hydroxy group, a nitro group, an amino group and the like.

As the optionally substituted thiol group, for example, a thiol group which may be substituted by an “alkyl group having 1 to 10 carbon atoms”, “alkenyl group having 2 to 10 carbon atoms”, “cycloalkyl group having 3 to 10 carbon atoms”, “cycloalkenyl group having 3 to 10 carbon atoms”, “aryl group having 6 to 14 carbon atoms”, “aralkyl group having 7 to 13 carbon atoms” or “acyl group having 1 to 13 carbon atoms”, each of which may be substituted, can be mentioned.

As used herein, as the “alkyl group having 1 to 10 carbon atoms”, “alkenyl group having 2 to 10 carbon atoms”, “cycloalkyl group having 3 to 10 carbon atoms”, “cycloalkenyl group having 3 to 10 carbon atoms”, “aryl group having 6 to 14 carbon atoms”, “aralkyl group having 7 to 13 carbon atoms” and “acyl group having 1 to 13 carbon atoms”, those exemplified as the substituent for ring A can be mentioned.

These “alkyl group having 1 to 10 carbon atoms”, “alkenyl group having 2 to 10 carbon atoms”, “cycloalkyl group having 3 to 10 carbon atoms”, “cycloalkenyl group having 3 to 10 carbon atoms”, “aryl group having 6 to 14 carbon atoms”, “aralkyl group having 7 to 13 carbon atoms” and “acyl group having 1 to 13 carbon atoms” may have 1 to 3 substituents at substitutable positions. As such substituents, for example, a halogen atom (e.g., fluorine, chlorine, bromine, iodine), a C₁₋₆ alkoxy group (e.g., methoxy, ethoxy) which may be substituted by 1 to 3 halogen atoms (e.g., fluorine, chlorine, bromine, iodine), a hydroxy group, a nitro group, an amino group and the like can be mentioned.

As the substituted thiol group, for example, an alkylthio group, an alkenylthio group, a cycloalkylthio group, a cycloalkenylthio group, an arylthio group, an aralkylthio group, an acylthio group and the like, each of which may be substituted, can be mentioned.

Preferable examples of the alkylthio group include an alkylthio group having 1 to 10 carbon atoms, such as methylthio, ethylthio, propylthio, isopropylthio, butylthio, isobutylthio, sec.-butylthio, t.-butylthio, pentylthio, isopentylthio, neopentylthio, hexylthio, heptylthio, nonylthio and the like.

Preferable examples of the alkenylthio group include an alkenylthio group having 2 to 10 carbon atoms, such as allylthio, crotylthio, 2-pentenylthio, 3-hexenylthio and the like.

Preferable examples of the cycloalkylthio group include a cycloalkylthio group having 3 to 10 carbon atoms, such as cyclobutylthio, cyclopentylthio, cyclohexylthio and the like.

Preferable examples of the cycloalkenylthio group include a cycloalkenylthio group having 3 to 10 carbon atoms, such as 2-cyclopentenylthio, 2-cyclohexenylthio and the like.

Preferable examples of the arylthio group include an arylthio group having 6 to 14 carbon atoms, such as phenylthio, naphthylthio and the like.

Preferable examples of the aralkylthio group include an aralkylthio group having 7 to 13 carbon atoms, such as benzylthio, phenethylthio, naphthylmethylthio and the like.

Preferable examples of the acylthio group include an acylthio group having 2 to 13 carbon atoms, such as alkanoylthio group having 2 to 4 carbon atoms (e.g., acetylthio, propionylthio, butyrylthio, isobutyrylthio) and the like.

The above-mentioned alkylthio group, alkenylthio group, cycloalkylthio group, cycloalkenylthio group, arylthio group, aralkylthio group and acylthio group may have 1 to 3 substituents at substitutable positions. As such substituents, for example, a halogen atom (e.g., fluorine, chlorine, bromine, iodine), a C₁₋₆ alkoxy group (e.g., methoxy, ethoxy) which may be substituted by 1 to 3 halogen atoms (e.g., fluorine, chlorine, bromine, iodine), a hydroxy group, a nitro group, an amino group and the like can be mentioned.

As the esterified carboxyl group of the optionally esterified carboxyl group, for example, an alkoxycarbonyl group having 2 to 5 carbon atoms (e.g., methoxycarbonyl, ethoxycarbonyl, propoxycarbonyl, butoxycarbonyl), an aralkyloxycarbonyl group having 8 to 14 carbon atoms (e.g., benzyloxycarbonyl), an aryloxycarbonyl group having 7 to 15 carbon atoms (e.g., phenoxycarbonyl) and the like can be mentioned.

As the amidated carboxyl group of the optionally amidated carboxyl group, a group of the formula: —CON(R⁹) (R¹⁰) [wherein R⁹ and R¹⁰ are the same or different and each is a hydrogen atom, an optionally substituted hydrocarbon group or an optionally substituted heterocyclic group, or R⁹ and R¹⁰ may form, together with the adjacent nitrogen atom, an optionally substituted nitrogen-containing heterocyclic ring] can be mentioned.

As used herein, the hydrocarbon group of the “optionally substituted hydrocarbon group” represented by R⁹ and R¹⁰ is exemplified by the hydrocarbon groups exemplified for the aforementioned R⁷. The hydrocarbon group is preferably an alkyl group having 1 to 10 carbon atoms (preferably methyl, ethyl, propyl, isopropyl, butyl, tert-butyl), an alkynyl group having 2 to 10 carbon atoms (preferably 2-propynyl), a cycloalkyl group having 3 to 10 carbon atoms (preferably cyclopropyl, cyclohexyl), an aryl group having 6 to 14 carbon atoms (preferably phenyl), an aralkyl group having 7 to 13 carbon atoms (preferably benzyl, phenethyl, naphthylmethyl) and the like.

As the substituent of the “optionally substituted hydrocarbon group” represented by R⁹ and R¹⁰, for example, a halogen atom (e.g., fluorine, chlorine, bromine, iodine); a sulfo group; a cyano group; an azido group; a nitro group; a nitroso group; an aromatic heterocyclic group (e.g., thienyl, furyl, pyridyl, oxazolyl, thiazolyl); a non-aromatic heterocyclic group (e.g., tetrahydrofuryl, morpholino, thiomorpholino, piperidino, pyrrolidinyl, piperazinyl); an amino group which may be mono- or di-substituted by a substituent selected from alkyl group having 1 to 4 carbon atoms and acyl group having 2 to 8 carbon atoms (e.g., alkanoyl group); an amidino group; an acyl group having 2 to 8 carbon atoms (e.g., alkanoyl group); a carbamoyl group which may be mono- or di-substituted by alkyl group having 1 to 4 carbon atoms; a sulfamoyl group which may be mono- or di-substituted by alkyl group having 1 to 4 carbon atoms; a carboxyl group; an alkoxycarbonyl group having 2 to 8 carbon atoms; a hydroxy group; an alkoxy group having 1 to 6 carbon atoms which may be substituted by 1 to 3 halogen atoms (e.g., fluorine, chlorine, bromine, iodine); an alkenyloxy group having 2 to 5 carbon atoms which may be substituted by 1 to 3 halogen atoms (e.g., fluorine, chlorine, bromine, iodine); a cycloalkyloxy group having 3 to 7 carbon atoms; an aralkyloxy group having 7 to 13 carbon atoms; an aryloxy group having 6 to 14 carbon atoms (e.g., phenyloxy, naphthyloxy); a thiol group; an alkylthio group having 1 to 6 carbon atoms which may be substituted by 1 to 3 halogen atoms (e.g., fluorine, chlorine, bromine, iodine); an aralkylthio group having 7 to 13 carbon atoms; an arylthio group having 6 to 14 carbon atoms (e.g., phenylthio, naphthylthio) and the like can be mentioned. The number of the substituent is, for example, 1 to 3.

As the heterocyclic group of the “optionally substituted heterocyclic group” represented by R⁹ and R¹⁰, the heterocyclic group exemplified for the aforementioned R⁷ can be mentioned.

As the substituent for the heterocyclic group, for example, a halogen atom (e.g., fluorine, chlorine, bromine, iodine); a sulfo group; a cyano group; an azido group; a nitro group; a nitroso group; an alkyl group having 1 to 6 carbon atoms which may be substituted by 1 to 3 halogen atoms (e.g., fluorine, chlorine, bromine, iodine); an alkenyl group having 2 to 6 carbon atoms which may be substituted by 1 to 3 halogen atoms (e.g., fluorine, chlorine, bromine, iodine); a cycloalkyl group having 3 to 10 carbon atoms; an aryl group having 6 to 14 carbon atoms (e.g., phenyl, naphthyl); an aromatic heterocyclic group (e.g., thienyl, furyl, pyridyl, oxazolyl, thiazolyl); a non-aromatic heterocyclic group (e.g., tetrahydrofuryl, morpholino, thiomorpholino, piperidino, pyrrolidinyl, piperazinyl); an aralkyl group having 7 to 13 carbon atoms; an amino group which may be mono- or di-substituted by a substituent selected from alkyl group having 1 to 4 carbon atoms and acyl group having 2 to 8 carbon atoms (e.g., alkanoyl group); an amidino group; an acyl group having 2 to 8 carbon atoms (e.g., alkanoyl group); a carbamoyl group which may be mono- or di-substituted by alkyl group having 1 to 4 carbon atoms; a sulfamoyl group which may be mono- or di-substituted by alkyl group having 1 to 4 carbon atoms; a carboxyl group; an alkoxycarbonyl group having 2 to 8 carbon atoms; a hydroxy group; an alkoxy group having 1 to 6 carbon atoms which may be substituted by 1 to 3 halogen atoms (e.g., fluorine, chlorine, bromine, iodine); an alkenyloxy group having 2 to 5 carbon atoms which may be substituted by 1 to 3 halogen atoms (e.g., fluorine, chlorine, bromine, iodine); a cycloalkyloxy group having 3 to 7 carbon atoms; an aralkyloxy group having 7 to 13 carbon atoms; an aryloxy group having 6 to 14 carbon atoms (e.g., phenyloxy, naphthyloxy); a thiol group; an alkylthio group having 1 to 6 carbon atoms which may be substituted by 1 to 3 halogen atoms (e.g., fluorine, chlorine, bromine, iodine); an aralkylthio group having 7 to 13 carbon atoms; an arylthio group having 6 to 14 carbon atoms (e.g., phenylthio, naphthylthio) and the like can be mentioned. The number of substituent is, for example, 1 to 3.

As the nitrogen-containing heterocyclic ring formed by R⁹ and R¹⁰ together with the adjacent nitrogen atom, for example, a 5- to 8-membered nitrogen-containing heterocyclic ring which contains, besides carbon atom, at least one nitrogen atom and optionally 1 or 2 heteroatoms selected from oxygen atom, sulfur atom and nitrogen atom can be mentioned. Preferable examples of the nitrogen-containing heterocyclic ring include pyrrolidine, imidazolidine, pyrazolidine, piperidine, piperazine, morpholine, thiomorpholine, azepane and the like.

The nitrogen-containing heterocyclic ring may have 1 or 2 substituents at substitutable positions. As such substituent, a C₁₋₆ alkyl group (e.g., methyl, ethyl) which may be substituted by 1 to 3 halogen atoms (e.g., fluorine, chlorine, bromine, iodine); a C₇₋₁₄ aralkyl group (e.g., benzyl, diphenylmethyl); a C₆₋₁₄ aryl group (e.g., phenyl) which may be substituted by a substituent selected from a C₁₋₆ alkyl group (e.g., methyl, trifluoromethyl) which may be substituted by 1 to 3 halogen atoms (e.g., fluorine, chlorine, bromine, iodine), a halogen atom (e.g., fluorine, chlorine, bromine, iodine), C₁₋₆ alkoxy group (e.g., methoxy, ethoxy) or C₂₋₁₀ alkanoyl group (e.g., acetyl); a cyano group; a hydroxy group; a C₂₋₇ alkoxycarbonyl group (e.g., methoxycarbonyl, ethoxycarbonyl) and the like can be mentioned.

The substituent for ring A is preferably a halogen atom, an optionally substituted aliphatic hydrocarbon group, an optionally substituted aromatic hydrocarbon group, an optionally substituted hydroxy group, a optionally substituted thiol group, a nitro group, a cyano group or an optionally substituted amino group, more preferably

1) a halogen atom (e.g., fluorine, chlorine, bromine, iodine);

2) an alkyl group having 1 to 6 carbon atoms (e.g., methyl, ethyl, propyl, isopropyl, trifluoromethyl) which may be substituted by 1 to 3 halogen atoms (e.g., fluorine, chlorine, bromine, iodine);

3) an aryl group having 6 to 14 carbon atoms (e.g., phenyl);

4) an alkoxy group having 1 to 6 carbon atoms (e.g., methoxy, ethoxy, propoxy, isopropoxy, butoxy, trifluoromethoxy) which may be substituted by 1 to 3 halogen atoms (e.g., fluorine, chlorine, bromine, iodine);

5) an alkylthio group having 1 to 6 carbon atoms (e.g., methylthio) which may be substituted by 1 to 3 halogen atoms (e.g., fluorine, chlorine, bromine, iodine);

6) a nitro group;

7) a cyano group; or

8) an amino group (e.g., amino, acetylamino, propionylamino, butyrylamino, isobutyrylamino, methylsulfonylamino) which may be substituted by C₂₋₁₀ alkanoyl group or C₁₋₁₀ alkylsulfonyl group. The number of substituent is preferably 1 or 2.

The ring A is preferably an aromatic ring (preferably aromatic hydrocarbon, aromatic heterocyclic ring) which may have 1 to 3 substituents selected from a halogen atom, an optionally substituted aliphatic hydrocarbon group, an optionally substituted aromatic hydrocarbon group, an optionally substituted hydroxy group, an optionally substituted thiol group, a nitro group, a cyano group, an optionally substituted amino group and the like, more preferably an aromatic hydrocarbon having 6 to 14 carbon atoms (preferably benzene) or a 5- or 6-membered aromatic heterocyclic ring (preferably pyridine, pyrimidine, pyridazine, oxadiazole, thiadiazole; more preferably pyridine, pyridazine), each of which may have 1 to 3 substituents selected from

1) a halogen atom (e.g., fluorine, chlorine, bromine, iodine);

2) an alkyl group having 1 to 6 carbon atoms (e.g., methyl, ethyl, propyl, isopropyl, trifluoromethyl) which may be substituted by 1 to 3 halogen atoms (e.g., fluorine, chlorine, bromine, iodine);

3) an aryl group having 6 to 14 carbon atoms (e.g., phenyl);

4) an alkoxy group having 1 to 6 carbon atoms (e.g., methoxy, ethoxy, propoxy, isopropoxy, butoxy, trifluoromethoxy) which may be substituted by 1 to 3 halogen atoms (e.g., fluorine, chlorine, bromine, iodine);

5) an alkylthio group having 1 to 6 carbon atoms (e.g., methylthio) which may be substituted by 1 to 3 halogen atoms (e.g., fluorine, chlorine, bromine, iodine);

6) a nitro group;

7) a cyano group;

8) an amino group (e.g., amino, acetylamino, propionylamino, butyrylamino, isobutyrylamino, methylsulfonylamino) which may be substituted by C₂₋₁₀ alkanoyl group or C₁₋₁₀ alkylsulfonyl group; and the like.

As the 1,2-azole ring represented by ring B, for example, pyrazole, isoxazole, isothiazole and the like can be mentioned. Of these, pyrazole is preferable.

The 1,2-azole ring represented by ring B may have 1 to 3 (preferably 1 or 2) substituents at substitutable positions. As such substituent, “a halogen atom”, “a nitro group”, “a cyano group”, “an optionally substituted aliphatic hydrocarbon group”, “an optionally substituted alicyclic hydrocarbon group”, “an optionally substituted aromatic hydrocarbon group”, “an optionally substituted heterocyclic group”, “an optionally substituted acyl group”, “an optionally substituted amino group”, “an optionally substituted hydroxy group”, “an optionally substituted thiol group”, “an optionally esterified or amidated carboxyl group” and the like exemplified as the substituent for ring A can be mentioned.

The substituent for ring B is preferably “an optionally substituted aliphatic hydrocarbon group”, “an optionally substituted alicyclic hydrocarbon group”, “an optionally substituted aromatic hydrocarbon group”, “an optionally substituted hydroxy group” and the like, more preferably a hydrocarbon group such as aliphatic hydrocarbon group, alicyclic hydrocarbon group, aromatic hydrocarbon group and the like; an alkoxy group; an aralkyloxy group and the like.

Specific examples of the substituent include an alkyl group having 1 to 6 carbon atoms (e.g., methyl, ethyl, propyl, isopropyl, butyl, sec.-butyl, t.-butyl, 1-ethylpropyl, 1-methylbutyl), an alkoxy group having 1 to 6 carbon atoms (e.g., methoxy, ethoxy), an aralkyloxy group having 7 to 13 carbon atoms (e.g., benzyloxy), a hydroxy group, an aryl group having 6 to 14 carbon atoms (e.g., phenyl), a cycloalkyl group having 3 to 10 carbon atoms (e.g., cyclohexyl) and the like.

The ring B is preferably a 1,2-azole ring (preferably pyrazole, isoxazole, isothiazole) which may have 1 to 3 (preferably 1 or 2) substituents selected from an optionally substituted aliphatic hydrocarbon group, an optionally substituted alicyclic hydrocarbon group, an optionally substituted aromatic hydrocarbon group, an optionally substituted hydroxy group and the like; more preferably pyrazole or isoxazole (preferably pyrazole), each of which may have 1 to 3 (preferably 1 or 2) substituents selected from an alkyl group having 1 to 6 carbon atoms (e.g., methyl, ethyl, propyl, isopropyl, butyl, sec.-butyl, t.-butyl, 1-ethylpropyl, 1-methylbutyl), an alkoxy group having 1 to 6 carbon atoms (e.g., methoxy, ethoxy, propoxy, isopropoxy, butoxy), an aralkyloxy group having 7 to 13 carbon atoms (e.g., benzyloxy), a hydroxy group, an aryl group having 6 to 14 carbon atoms (e.g., phenyl), a cycloalkyl group having 3 to 10 carbon atoms (e.g., cyclohexyl) and the like.

When ring B is pyrazole, it is preferable that ring A and Xa, which are substituents on ring B, are substituted on the 1st and 4th position on the pyrazole, respectively.

Xa, Xb and Xc are the same or different and each is a bond, —O—, —S—, —SO—, —SO₂—, —CO—, —CS—, —CR¹(OR²)—, —NR³—, —CONR³— or —NR³CO— (R¹ is a hydrogen atom or an optionally substituted hydrocarbon group, R² is a hydrogen atom or a hydroxy-protecting group, R³ is a hydrogen atom, an optionally substituted hydrocarbon group or an amino-protecting group).

As the “optionally substituted hydrocarbon group” represented by R¹ or R³, those exemplified as the aforementioned R⁹ can be mentioned.

The “optionally substituted hydrocarbon group” is preferably an optionally substituted alkyl group having 1 to 6 carbon atoms (e.g., methyl, ethyl, propyl, isopropyl, butyl, isobutyl, sec.-butyl, t.-butyl). The alkyl group may have 1 to 3 substituents at substitutable positions, and as such substituent, for example, a halogen atom (e.g., fluorine, chlorine, bromine, iodine), an alkoxy group having 1 to 4 carbon atoms (e.g., methoxy, ethoxy, propoxy, isopropoxy, butoxy, isobutoxy, sec.-butoxy, t.-butoxy), a hydroxy group, a nitro group, an amino group, an acyl group having 1 to 4 carbon atoms (e.g., alkanoyl group having 1 to 4 carbon atoms such as formyl, acetyl, propionyl etc.) and the like can be mentioned.

As the hydroxy-protecting group represented by R², for example, a C₁₋₆alkyl group (e.g., methyl, ethyl, propyl, isopropyl, butyl, tert-butyl), a phenyl group, a trityl group, a C₇₋₁₀ aralkyl group (e.g., benzyl), a formyl group, a C₁₋₆ alkyl-carbonyl group (e.g., acetyl, propionyl), a benzoyl group, a C₇₋₁₀ aralkyl-carbonyl group (e.g., benzylcarbonyl), a 2-tetrahydropyranyl group, a 2-tetrahydrofuranyl group, a silyl group (e.g., trimethylsilyl, triethylsilyl, dimethylphenylsilyl, tert-butyldimethylsilyl, tert-butyldiethylsilyl), a C₂₋₆ alkenyl group (e.g., 1-allyl) and the like can be mentioned. These groups may be substituted by 1 to 3 substituents selected from a halogen atom (e.g., fluorine, chlorine, bromine, iodine), a C₁₋₆ alkyl group (e.g., methyl, ethyl, propyl), a C₁₋₆ alkoxy group (e.g., methoxy, ethoxy, propoxy), a nitro group and the like.

As the amino-protecting group represented by R³, for example, a formyl group, a C₁₋₆ alkyl-carbonyl group (e.g., acetyl, propionyl), a C₁₋₆ alkoxy-carbonyl group (e.g., methoxycarbonyl, ethoxycarbonyl, tert-butoxycarbonyl), a benzoyl group, a C₇₋₁₀ aralkyl-carbonyl group (e.g., benzylcarbonyl), a C₇₋₁₄ aralkyloxy-carbonyl group (e.g., benzyloxycarbonyl, a 9-fluorenylmethoxycarbonyl), a trityl group, a phthaloyl group, an N,N-dimethylaminomethylene group, a silyl group (e.g., trimethylsilyl, triethylsilyl, dimethylphenylsilyl, tert-butyldimethylsilyl, tert-butyldiethylsilyl), a C₂₋₆ alkenyl group (e.g., 1-allyl) and the like can be mentioned. These groups may be substituted by 1 to 3 substituents selected from a halogen atom (e.g., fluorine, chlorine, bromine, iodine), a C₁₋₆ alkoxy group (e.g., methoxy, ethoxy, propoxy), a nitro group and the like.

R¹ and R³ are preferably a hydrogen atom or an alkyl group having 1 to 6 carbon atoms, R² is preferably a hydrogen atom.

Xa is preferably a bond, —O—, —NR³— or —CONR³— (R³ is preferably a hydrogen atom or an alkyl group having 1 to 6 carbon atoms), more preferably a bond or —O—, particularly preferably a bond.

Xb is preferably —O—, —S—, —SO—, —SO₂—, —CO—, —CS—, —CR¹(OR²)—, —NR³—, —CONR³— or —NR³CO— (R¹ and R³ are preferably a hydrogen atom or an alkyl group having 1 to 6 carbon atoms; and R² is preferably a hydrogen atom), more preferably a bond or —O—, particularly preferably —O—.

Xc is preferably a bond or —O—, more preferably a bond.

As the “divalent aliphatic hydrocarbon residue having 1 to 20 carbon atoms” represented by Ya, Yb and Yc, for example, an alkylene having 1 to 20 carbon atoms, an alkenylene having 2 to 20 carbon atoms, an alkynylene having 2 to 20 carbon atoms and the like can be mentioned.

The “divalent aliphatic hydrocarbon residue having 1 to 20 carbon atoms” is preferably a divalent aliphatic hydrocarbon group having 1 to 6 carbon atoms, more preferably

(1) a C₁₋₆ alkylene (e.g., —CH₂—, —(CH₂)₂—, —(CH₂)₃—, —(CH₂)₄—, —(CH₂)₅—, —(CH₂)₆—, —CH(CH₃)—, —C(CH₃)₂—, —(CH(CH₃))₂—, —(CH₂)₂C(CH₃)₂—, —(CH₂)₃C(CH₃)₂— and the like);

(2) a C₂₋₆ alkenylene (e.g., —CH═CH—, —CH₂—CH═CH—, —C(CH₃)₂—CH═CH—, —CH₂—CH═CH—CH₂—, —CH₂—CH₂—CH═CH—, —CH═CH—CH═CH—, —CH═CH—CH₂—CH₂—CH₂— and the like); or

(3) a C₂₋₆ alkynylene (e.g., —C≡C—, —CH₂—C≡C—, —CH₂—C≡C—CH₂—CH₂— and the like) and the like.

Of these, a C₁₋₆ alkylene and a C₂₋₆ alkenylene are preferable.

Ya is preferably a C₁₋₆ alkylene or a C₂₋₆ alkenylene, more preferably a C₁₋₆ alkylene (preferably —CH₂—, —(CH₂)₂—, —(CH₂)₃— and the like). When Xa and Xb are bonds, Ya is preferably a C₃₋₆ alkylene or a C₃₋₆ alkenylene.

Yb is preferably a bond, a C₁₋₆ alkylene or a C₂₋₆ alkenylene, more preferably a bond.

Yc is preferably a bond, a C₁₋₆ alkylene or a C₂₋₆ alkenylene, more preferably a C₁₋₆ alkylene or a C₂₋₆ alkenylene. Especially, a C₁₋₆ alkylene (preferably —CH₂— and the like) is preferable.

As the monocyclic aromatic ring represented by ring C, monocyclic ring from among the aromatic hydrocarbon and aromatic heterocyclic ring exemplified for the aforementioned ring A can be mentioned.

The monocyclic aromatic ring is preferably a benzene or a 5- or 6-membered monocyclic aromatic heterocyclic ring, more preferably benzene, pyrazole, pyridine and the like. Of these, benzene, pyrazole and the like are preferable. Especially, benzene is preferable.

The monocyclic aromatic ring represented by ring C may have 1 to 3 substituents at substitutable positions. As the substituent, “a halogen atom”, “a nitro group”, “a cyano group”, “an optionally substituted aliphatic hydrocarbon group”, “an optionally substituted alicyclic hydrocarbon group”, “an optionally substituted aromatic hydrocarbon group”, “an optionally substituted heterocyclic group”, “an optionally substituted acyl group”, “an optionally substituted amino group”, “an optionally substituted hydroxy group”, “an optionally substituted thiol group”, “an optionally esterified or amidated carboxyl group” and the like exemplified as substituent for ring A can be mentioned.

The substituent for ring C is preferably a halogen atom, an optionally substituted aliphatic hydrocarbon group, an optionally substituted aromatic hydrocarbon group, an optionally substituted hydroxy group, an optionally substituted thiol group, a cyano group, an optionally substituted alicyclic hydrocarbon group and the like, more preferably

1) a halogen atom (e.g., fluorine, chlorine, bromine, iodine);

2) an alkyl group having 1 to 6 carbon atoms (e.g., methyl, ethyl, propyl, isopropyl, trifluoromethyl) which may be substituted by 1 to 3 halogen atoms (e.g., fluorine, chlorine, bromine, iodine);

3) an aryl group having 6 to 14 carbon atoms (e.g., phenyl) which may be substituted by 1 to 3 halogen atoms (e.g., fluorine, chlorine, bromine, iodine);

4) an alkoxy group having 1 to 6 carbon atoms (e.g., methoxy, ethoxy, propoxy, isopropoxy, butoxy, trifluoromethoxy) which may be substituted by 1 to 3 halogen atoms (e.g., fluorine, chlorine, bromine, iodine);

5) an alkylthio group having 1 to 6 carbon atoms (e.g., methylthio) which may be substituted by 1 to 3 halogen atoms (e.g., fluorine, chlorine, bromine, iodine);

6) a hydroxy group;

7) an aralkyloxy group having 7 to 13 carbon atoms (e.g., benzyloxy);

8) a cyano group;

9) a cycloalkyl group having 3 to 10 carbon atoms (e.g., cyclohexyl); and the like.

The ring C is preferably a benzene or a 5- or 6-membered monocyclic aromatic heterocyclic ring (preferably pyrazole or pyridine, more preferably pyrazole), each of which may have 1 to 3 substituents selected from a halogen atom, an optionally substituted aliphatic hydrocarbon group, an optionally substituted aromatic hydrocarbon group, an optionally substituted hydroxy group, an optionally substituted thiol group, a cyano group, an optionally substituted alicyclic hydrocarbon group and the like; more preferably a benzene or a 5- or 6-membered monocyclic aromatic heterocyclic ring (preferably pyrazole or pyridine, more preferably pyrazole), each of which may have 1 to 3 substituents selected from

1) a halogen atom (e.g., fluorine, chlorine, bromine, iodine);

2) an alkyl group having 1 to 6 carbon atoms (e.g., methyl, ethyl, propyl, isopropyl, trifluoromethyl) which may be substituted by 1 to 3 halogen atoms (e.g., fluorine, chlorine, bromine, iodine);

3) an aryl group having 6 to 14 carbon atoms (e.g., phenyl) which may be substituted by 1 to 3 halogen atoms (e.g., fluorine, chlorine, bromine, iodine);

4) an alkoxy group having 1 to 6 carbon atoms (e.g., methoxy, ethoxy, propoxy, isopropoxy, butoxy, trifluoromethoxy) which may be substituted by 1 to 3 halogen atoms (e.g., fluorine, chlorine, bromine, iodine);

5) an alkylthio group having 1 to 6 carbon atoms (e.g., methylthio) which may be substituted by 1 to 3 halogen atoms (e.g., fluorine, chlorine, bromine, iodine);

6) a hydroxy group;

7) an aralkyloxy group having 7 to 13 carbon atoms (e.g., benzyloxy);

8) a cyano group;

9) a cycloalkyl group having 3 to 10 carbon atoms (e.g., cyclohexyl); and the like.

R represents —OR⁴ (R⁴ is a hydrogen atom or an optionally substituted hydrocarbon group) or —NR⁵R⁶ (R⁵ and R⁶ are the same or different and each is a hydrogen atom, an optionally substituted hydrocarbon group or an optionally substituted heterocyclic group, or R⁵ and R⁶ form, together with the adjacent nitrogen atom, an optionally substituted heterocyclic ring).

As the “optionally substituted hydrocarbon group” represented by R⁴, R⁵ and R⁶, those exemplified as the aforementioned R⁹ can be mentioned.

The “optionally substituted hydrocarbon group” is preferably an optionally substituted alkyl group having 1 to 6 carbon atoms (e.g., methyl, ethyl, propyl, isopropyl, butyl, isobutyl, sec.-butyl, t.-butyl).

As the “optionally substituted heterocyclic group” represented by R⁵ and R⁶, those exemplified as the aforementioned R⁹ can be mentioned.

As the “optionally substituted heterocyclic ring” formed by R⁵ and R⁶ together with the adjacent nitrogen atom, the aforementioned optionally substituted nitrogen-containing heterocyclic ring” formed by R⁹ and R¹⁰ together with the adjacent nitrogen atom can be mentioned.

R is preferably —OR⁴ (R⁴ is a hydrogen atom or an optionally substituted hydrocarbon group). As used herein, R⁴ is preferably a hydrogen atom or an alkyl group having 1 to 6 carbon atoms (preferably methyl, ethyl and the like), more preferably a hydrogen atom.

In the formula (I),

(1) when the 1,2-azole ring represented by ring B is pyrazole, ring C is not thiadiazole or oxadiazole;

(2) when the 1,2-azole ring represented by ring B is isoxazole, ring C is not an optionally substituted pyridone;

(3) when the 1,2-azole ring represented by ring B is pyrazole and Xa and Xb are bonds, ring C is not a benzene ring.

In the formula (Ib),

when the 1,2-azole ring represented by ring B is isoxazole, ring C is not an optionally substituted pyridone.

Preferable examples of the compound represented by the formula (I) include the following compounds.

[Compound A]

A compound wherein

ring A is an aromatic hydrocarbon having 6 to 14 carbon atoms (preferably benzene) or a 5- or 6-membered aromatic heterocyclic ring (preferably pyridine), each of which may have 1 to 3 substituents selected from

1) a halogen atom (e.g., fluorine, chlorine, bromine, iodine);

2) an alkyl group having 1 to 6 carbon atoms (e.g., methyl, ethyl, propyl, isopropyl, trifluoromethyl) which may be substituted by 1 to 3 halogen atoms (e.g., fluorine, chlorine, bromine, iodine);

3) an aryl group having 6 to 14 carbon atoms (e.g., phenyl);

4) an alkoxy group having 1 to 6 carbon atoms (e.g., methoxy, ethoxy, propoxy, isopropoxy, butoxy, trifluoromethoxy) which may be substituted by 1 to 3 halogen atoms (e.g., fluorine, chlorine, bromine, iodine);

5) an alkylthio group having 1 to 6 carbon atoms (e.g., methylthio) which may be substituted by 1 to 3 halogen atoms (e.g., fluorine, chlorine, bromine, iodine); and the like; ring B is pyrazole or isoxazole (preferably pyrazole), each of which may have 1 to 3 (preferably 1 or 2) substituents selected from an alkyl group having 1 to 6 carbon atoms (e.g., methyl, ethyl, propyl, isopropyl), an alkoxy group having 1 to 6 carbon atoms (e.g., methoxy, ethoxy, propoxy, isopropoxy, butoxy), an aralkyloxy group having 7 to 13 carbon atoms (e.g., benzyloxy) and the like;

Xa is a bond or —O—;

Xb is a bond or —O—;

Xc is a bond or —O—;

Ya is a C₁₋₆ alkylene or a C₂₋₆ alkenylene;

Yb is a bond;

Yc is a bond, a C₁₋₆ alkylene or a C₂₋₆ alkenylene; ring C is benzene optionally having 1 to 3 substituents selected from

1) a halogen atom (e.g., fluorine, chlorine, bromine, iodine);

2) an alkyl group having 1 to 6 carbon atoms (e.g., methyl, ethyl, propyl, isopropyl, trifluoromethyl) which may be substituted by 1 to 3 halogen atoms (e.g., fluorine, chlorine, bromine, iodine);

3) an aryl group having 6 to 14 carbon atoms (e.g., phenyl);

4) an alkoxy group having 1 to 6 carbon atoms (e.g., methoxy, ethoxy, propoxy, isopropoxy, butoxy, trifluoromethoxy) which may be substituted by 1 to 3 halogen atoms (e.g., fluorine, chlorine, bromine, iodine);

5) an alkylthio group having 1 to 6 carbon atoms (e.g., methylthio) which may be substituted by 1 to 3 halogen atoms (e.g., fluorine, chlorine, bromine, iodine); and the like; and

R is —OR⁴ (R⁴ is preferably a hydrogen atom or an alkyl group having 1 to 6 carbon atoms).

[Compound B]

A compound wherein

ring A is an aromatic hydrocarbon having 6 to 14 carbon atoms (preferably benzene) or a 5- or 6-membered aromatic heterocyclic ring (preferably pyridine), each of which may have 1 to 3 substituents selected from

1) a halogen atom (e.g., fluorine, chlorine, bromine, iodine);

2) an alkyl group having 1 to 6 carbon atoms (e.g., methyl, ethyl, propyl, isopropyl, trifluoromethyl) which may be substituted by 1 to 3 halogen atoms (e.g., fluorine, chlorine, bromine, iodine);

3) an aryl group having 6 to 14 carbon atoms (e.g., phenyl);

4) an alkoxy group having 1 to 6 carbon atoms (e.g., methoxy, ethoxy, propoxy, isopropoxy, butoxy, trifluoromethoxy) which may be substituted by 1 to 3 halogen atoms (e.g., fluorine, chlorine, bromine, iodine);

5) an alkylthio group having 1 to 6 carbon atoms (e.g., methylthio) which may be substituted by 1 to 3 halogen atoms (e.g., fluorine, chlorine, bromine, iodine) and the like;

ring B is pyrazole or isoxazole (preferably pyrazole), each of which may have 1 to 3 (preferably 1 or 2) substituents selected from an alkyl group having 1 to 6 carbon atoms (e.g., methyl, ethyl, propyl, isopropyl), an alkoxy group having 1 to 6 carbon atoms (e.g., methoxy, ethoxy, propoxy, isopropoxy, butoxy), an aralkyloxy group having 7 to 13 carbon atoms (e.g., benzyloxy); and the like;

Xa is a bond or —O—;

Xb is a bond or —O—;

Xc is a bond or —O—;

Ya is a C₁₋₆ alkylene or a C₂₋₆ alkenylene;

Yb is a bond;

Yc is a bond, a C₁₋₆ alkylene or a C₂₋₆ alkenylene;

ring C is a 5- or 6-membered monocyclic aromatic heterocyclic ring (preferably pyrazole), which may have 1 to 3 substituents selected from

1) a halogen atom (e.g., fluorine, chlorine, bromine, iodine);

2) an alkyl group having 1 to 6 carbon atoms (e.g., methyl, ethyl, propyl, isopropyl, trifluoromethyl) which may be substituted by 1 to 3 halogen atoms (e.g., fluorine, chlorine, bromine, iodine);

3) an aryl group having 6 to 14 carbon atoms (e.g., phenyl);

4) an alkoxy group having 1 to 6 carbon atoms (e.g., methoxy, ethoxy, propoxy, isopropoxy, butoxy, trifluoromethoxy) which may be substituted by 1 to 3 halogen atoms (e.g., fluorine, chlorine, bromine, iodine);

5) an alkylthio group having 1 to 6 carbon atoms (e.g., methylthio) which may be substituted by 1 to 3 halogen atoms (e.g., fluorine, chlorine, bromine, iodine); and the like; and

R is —OR⁴ (R⁴ is preferably a hydrogen atom or an alkyl group having 1 to 6 carbon atoms).

[Compound C]

A compound wherein

ring A is an aromatic hydrocarbon having 6 to 14 carbon atoms (preferably benzene), a 5- or 6-membered aromatic heterocyclic ring (preferably pyridine) or an alicyclic hydrocarbon having 3 to 12 carbon atoms (preferably cyclopentane), each of which may have 1 to 3 substituents selected from

1) a halogen atom (e.g., fluorine, chlorine, bromine, iodine);

2) an alkyl group having 1 to 6 carbon atoms (e.g., methyl, ethyl, propyl, isopropyl, trifluoromethyl) which may be substituted by 1 to 3 halogen atoms (e.g., fluorine, chlorine, bromine, iodine);

3) an aryl group having 6 to 14 carbon atoms (e.g., phenyl);

4) an alkoxy group having 1 to 6 carbon atoms (e.g., methoxy, ethoxy, propoxy, isopropoxy, butoxy, trifluoromethoxy) which may be substituted by 1 to 3 halogen atoms (e.g., fluorine, chlorine, bromine, iodine);

5) an alkylthio group having 1 to 6 carbon atoms (e.g., methylthio) which may be substituted by 1 to 3 halogen atoms (e.g., fluorine, chlorine, bromine, iodine); and the like;

ring B is a pyrazole or isoxazole (preferably pyrazole), each of which may have 1 to 3 (preferably 1 or 2) substituents selected from an alkyl group having 1 to 6 carbon atoms (e.g., methyl, ethyl, propyl, isopropyl), an alkoxy group having 1 to 6 carbon atoms (e.g., methoxy, ethoxy, propoxy, isopropoxy, butoxy), an aralkyloxy group having 7 to 13 carbon atoms (e.g., benzyloxy), a hydroxy group, an aryl group having 6 to 14 carbon atoms (e.g., phenyl) and the like;

Xa is a bond or —O—;

Xb is a bond or —O—;

Xc is a bond or —O—;

Ya is a C₁₋₆ alkylene or a C₂₋₆ alkenylene;

Yb is a bond;

Yc is a bond, a C₁₋₆ alkylene or a C₂₋₆ alkenylene;

ring C is a benzene optionally having 1 to 3 substituents selected from

1) a halogen atom (e.g., fluorine, chlorine, bromine, iodine);

2) an alkyl group having 1 to 6 carbon atoms (e.g., methyl, ethyl, propyl, isopropyl, trifluoromethyl) which may be substituted by 1 to 3 halogen atoms (e.g., fluorine, chlorine, bromine, iodine);

3) an aryl group having 6 to 14 carbon atoms (e.g., phenyl) which may be substituted by 1 to 3 halogen atoms (e.g., fluorine, chlorine, bromine, iodine);

4) an alkoxy group having 1 to 6 carbon atoms (e.g., methoxy, ethoxy, propoxy, isopropoxy, butoxy, trifluoromethoxy) which may be substituted by 1 to 3 halogen atoms (e.g., fluorine, chlorine, bromine, iodine);

5) an alkylthio group having 1 to 6 carbon atoms (e.g., methylthio) which may be substituted by 1 to 3 halogen atoms (e.g., fluorine, chlorine, bromine, iodine);

6) a hydroxy group;

7) an aralkyloxy group having 7 to 13 carbon atoms (e.g., benzyloxy); and the like; and

R is —OR⁴ (R⁴ is preferably a hydrogen atom or an alkyl group having 1 to 6 carbon atoms).

[Compound D]

A compound wherein ring A is an aromatic hydrocarbon having 6 to 14 carbon atoms (preferably benzene), a 5- or 6-membered aromatic heterocyclic ring (preferably pyridine) or an alicyclic hydrocarbon having 3 to 12 carbon atoms (preferably cyclopentane), each of which may have 1 to 3 substituents selected from

1) a halogen atom (e.g., fluorine, chlorine, bromine, iodine);

2) an alkyl group having 1 to 6 carbon atoms (e.g., methyl, ethyl, propyl, isopropyl, trifluoromethyl) which may be substituted by 1 to 3 halogen atoms (e.g., fluorine, chlorine, bromine, iodine);

3) an aryl group having 6 to 14 carbon atoms (e.g., phenyl);

4) an alkoxy group having 1 to 6 carbon atoms (e.g., methoxy, ethoxy, propoxy, isopropoxy, butoxy, trifluoromethoxy) which may be substituted by 1 to 3 halogen atoms (e.g., fluorine, chlorine, bromine, iodine);

5) an alkylthio group having 1 to 6 carbon atoms (e.g., methylthio) which may be substituted by 1 to 3 halogen atoms (e.g., fluorine, chlorine, bromine, iodine); and the like;

ring B is a pyrazole or isoxazole (preferably pyrazole), each of which may have 1 to 3 (preferably 1 or 2) substituents selected from an alkyl group having 1 to 6 carbon atoms (e.g., methyl, ethyl, propyl, isopropyl), an alkoxy group having 1 to 6 carbon atoms (e.g., methoxy, ethoxy, propoxy, isopropoxy, butoxy), an aralkyloxy group having 7 to 13 carbon atoms (e.g., benzyloxy), a hydroxy group, an aryl group having 6 to 14 carbon atoms (e.g., phenyl) and the like;

Xa is a bond or —O—;

Xb is a bond or —O—;

Xc is a bond or —O—;

Ya is a C₁₋₆ alkylene or a C₂₋₆ alkenylene;

Yb is a bond;

Yc is a bond, a C₁₋₆ alkylene or a C₂₋₆ alkenylene;

ring C is a 5- or 6-membered monocyclic aromatic heterocyclic ring (preferably pyrazole) optionally having 1 to 3 substituents selected from

1) a halogen atom (e.g., fluorine, chlorine, bromine, iodine);

2) an alkyl group having 1 to 6 carbon atoms (e.g., methyl, ethyl, propyl, isopropyl, trifluoromethyl) which may be substituted by 1 to 3 halogen atoms (e.g., fluorine, chlorine, bromine, iodine);

3) an aryl group having 6 to 14 carbon atoms (e.g., phenyl) which may be substituted by 1 to 3 halogen atoms (e.g., fluorine, chlorine, bromine, iodine);

4) an alkoxy group having 1 to 6 carbon atoms (e.g., methoxy, ethoxy, propoxy, isopropoxy, butoxy, trifluoromethoxy) which may be substituted by 1 to 3 halogen atoms (e.g., fluorine, chlorine, bromine, iodine);

5) an alkylthio group having 1 to 6 carbon atoms (e.g., methylthio) which may be substituted by 1 to 3 halogen atoms (e.g., fluorine, chlorine, bromine, iodine);

6) a hydroxy group;

7) an aralkyloxy group having 7 to 13 carbon atoms (e.g., benzyloxy); and the like; and

R is —OR⁴ (R⁴ is preferably a hydrogen atom or an alkyl group having 1 to 6 carbon atoms).

[Compound E]

A compound wherein ring A is an aromatic hydrocarbon having 6 to 14 carbon atoms (preferably benzene), a 5- or 6-membered aromatic heterocyclic ring (preferably pyridine, pyrimidine, pyridazine, oxadiazole, thiadiazole) or an alicyclic hydrocarbon having 3 to 12 carbon atoms (preferably cyclopentane), each of which may have 1 to 3 substituents selected from

1) a halogen atom (e.g., fluorine, chlorine, bromine, iodine);

2) an alkyl group having 1 to 6 carbon atoms (e.g., methyl, ethyl, propyl, isopropyl, trifluoromethyl) which may be substituted by 1 to 3 halogen atoms (e.g., fluorine, chlorine, bromine, iodine);

3) an aryl group having 6 to 14 carbon atoms (e.g., phenyl);

4) an alkoxy group having 1 to 6 carbon atoms (e.g., methoxy, ethoxy, propoxy, isopropoxy, butoxy, trifluoromethoxy) which may be substituted by 1 to 3 halogen atoms (e.g., fluorine, chlorine, bromine, iodine);

5) an alkylthio group having 1 to 6 carbon atoms (e.g., methylthio) which may be substituted by 1 to 3 halogen atoms (e.g., fluorine, chlorine, bromine, iodine);

6) a nitro group;

7) a cyano group;

8) an amino group (e.g., amino, acetylamino, propionylamino, butyrylamino, isobutyrylamino, methylsulfonylamino) which may be substituted by a C₂₋₁₀ alkanoyl group or a C₁₋₁₀ alkylsulfonyl group; and the like;

ring B is pyrazole or isoxazole (preferably pyrazole), each of which may have 1 to 3 (preferably 1 or 2) substituents selected from an alkyl group having 1 to 6 carbon atoms (e.g., methyl, ethyl, propyl, isopropyl, butyl, sec.-butyl, t.-butyl, 1-ethylpropyl, 1-methylbutyl), an alkoxy group having 1 to 6 carbon groups (e.g., methoxy, ethoxy, propoxy, isopropoxy, butoxy), an aralkyloxy group having 7 to 13 carbon atoms (e.g., benzyloxy), a hydroxy group, an aryl group having 6 to 14 carbon atoms (e.g., phenyl), a cycloalkyl group having 3 to 10 carbon atoms (e.g., cyclohexyl) and the like;

Xa is a bond or —O—;

Xb is a bond or —O—;

Xc is a bond or —O—;

Ya is a C₁₋₆ alkylene or a C₂₋₆ alkenylene;

Yb is a bond;

Yc is a bond, C₁₋₆ alkylene or a C₂₋₆ alkenylene;

ring C is benzene optionally having 1 to 3 substituents selected from

1) a halogen atom (e.g., fluorine, chlorine, bromine, iodine);

2) an alkyl group having 1 to 6 carbon atoms (e.g., methyl, ethyl, propyl, isopropyl, trifluoromethyl) which may be substituted by 1 to 3 halogen atoms (e.g., fluorine, chlorine, bromine, iodine);

3) an aryl group having 6 to 14 carbon atoms (e.g., phenyl) which may be substituted by 1 to 3 halogen atoms (e.g., fluorine, chlorine, bromine, iodine);

4) an alkoxy group having 1 to 6 carbon atoms (e.g., methoxy, ethoxy, propoxy, isopropoxy, butoxy, trifluoromethoxy) which may be substituted by 1 to 3 halogen atoms (e.g., fluorine, chlorine, bromine, iodine);

5) an alkylthio group having 1 to 6 carbon atoms (e.g., methylthio) which may be substituted by 1 to 3 halogen atoms (e.g., fluorine, chlorine, bromine, iodine);

6) a hydroxy group;

7) an aralkyloxy group having 7 to 13 carbon atoms (e.g., benzyloxy);

8) a cyano group;

9) a cycloalkyl group having 3 to 10 carbon atoms (e.g., cyclohexyl); and the like; and

R is —OR⁴ (R⁴ is preferably a hydrogen atom or an alkyl group having 1 to 6 carbon atoms).

[Compound F]

A compound wherein ring A is an aromatic hydrocarbon having 6 to 14 carbon atoms (preferably benzene), a 5- or 6-membered aromatic heterocyclic ring (preferably pyridine, pyrimidine, pyridazine, oxadiazole, thiadiazole) or an alicyclic hydrocarbon having 3 to 12 carbon atoms (preferably cyclopentane), each of which may have 1 to 3 substituents selected from

1) a halogen atom (e.g., fluorine, chlorine, bromine, iodine);

2) an alkyl group having 1 to 6 carbon atoms (e.g., methyl, ethyl, propyl, isopropyl, trifluoromethyl) which may be substituted by 1 to 3 halogen atoms (e.g., fluorine, chlorine, bromine, iodine);

3) an aryl group having 6 to 14 carbon atoms (e.g., phenyl);

4) an alkoxy group having 1 to 6 carbon atoms (e.g., methoxy, ethoxy, propoxy, isopropoxy, butoxy, trifluoromethoxy) which may be substituted by 1 to 3 halogen atoms (e.g., fluorine, chlorine, bromine, iodine);

5) an alkylthio group having 1 to 6 carbon atoms (e.g., methylthio) which may be substituted by 1 to 3 halogen atoms (e.g., fluorine, chlorine, bromine, iodine);

6) a nitro group;

7) a cyano group;

8) an amino group (e.g., amino, acetylamino, propionylamino, butyrylamino, isobutyrylamino, methylsulfonylamino) which may be substituted by a C₂₋₁₀ alkanoyl group or a C₁₋₁₀ alkylsulfonyl group; and the like;

ring B is pyrazole or isoxazole (preferably pyrazole), each of which may have 1 to 3 (preferably 1 or 2) substituents selected from an alkyl group having 1 to 6 carbon atoms (e.g., methyl, ethyl, propyl, isopropyl, butyl, sec.-butyl, t.-butyl, 1-ethylpropyl, 1-methylbutyl), alkoxy group having 1 to 6 carbon atoms (e.g., methoxy, ethoxy, propoxy, isopropoxy, butoxy), aralkyloxy group having 7 to 13 carbon atoms (e.g., benzyloxy), hydroxy group, aryl group having 6 to 14 carbon atoms (e.g., phenyl), cycloalkyl group having 3 to 10 carbon atoms (e.g., cyclohexyl) and the like;

Xa is a bond or —O—;

Xb is a bond or —O—;

Xc is a bond or —O—;

Ya is a C₁₋₆ alkylene or C₂₋₆ alkenylene;

Yb is a bond;

Yc is a bond, a C₁₋₆ alkylene or a C₂₋₆ alkenylene;

ring C is a 5- or 6-membered monocyclic aromatic heterocyclic ring (preferably pyrazole) optionally having 1 to 3 substituents selected from

1) a halogen atom (e.g., fluorine, chlorine, bromine, iodine);

2) an alkyl group having 1 to 6 carbon atoms (e.g., methyl, ethyl, propyl, isopropyl, trifluoromethyl) which may be substituted by 1 to 3 halogen atoms (e.g., fluorine, chlorine, bromine, iodine);

3) an aryl group having 6 to 14 carbon atoms (e.g., phenyl) which may be substituted by 1 to 3 halogen atoms (e.g., fluorine, chlorine, bromine, iodine);

4) an alkoxy group having 1 to 6 carbon atoms (e.g., methoxy, ethoxy, propoxy, isopropoxy, butoxy, trifluoromethoxy) which may be substituted by 1 to 3 halogen atoms (e.g., fluorine, chlorine, bromine, iodine);

5) an alkylthio group having 1 to 6 carbon atoms (e.g., methylthio) which may be substituted by 1 to 3 halogen atoms (e.g., fluorine, chlorine, bromine, iodine);

6) a hydroxy group;

7) an aralkyloxy group having 7 to 13 carbon atoms (e.g., benzyloxy);

8) a cyano group;

9) a cycloalkyl group having 3 to 10 carbon atoms (e.g., cyclohexyl); and the like; and

R is —OR⁴ (R⁴ is preferably a hydrogen atom or an alkyl group having 1 to 6 carbon atoms).

[Compound G]

3-[1-phenyl-3-(4-{3-[4-(trifluoromethyl)phenyl]-5-isoxazolyl}butoxy)-1H-pyrazol-5-yl]propionic acid (Example 11);
2-[3-(3-{3-ethoxy-1-[5-(trifluoromethyl)-2-pyridyl]-1H-pyrazol-4-yl}propoxy)phenoxy]-2-methylpropionic acid (Example 29);
3-[2-ethoxy-4-(3-[(3-ethoxy-1-[5-(trifluoromethyl)-2-pyridyl]-1H-pyrazol-4-yl]propoxy)phenyl]propionic acid (Example 35);
3-[3-(3-{3-ethoxy-1-[5-(trifluoromethyl)-2-pyridyl]-1H-pyrazol-4-yl}propoxy)-1-phenyl-1H-pyrazol-5-yl]propionic acid (Example 42);
[1-phenyl-3-(4-{3-propyl-1-[5-(trifluoromethyl)-2-pyridinyl]-1H-pyrazol-4-yl}butoxy)-1H-pyrazol-4-yl]acetic acid (Example 66);
[2-(3-{3-isopropyl-1-[5-(trifluoromethyl)-2-pyridyl]-1H-pyrazol-4-yl}propoxy)-3-methoxyphenyl]acetic acid (Example 181);
[2-(3-{3-(1-ethylpropyl)-1-[5-(trifluoromethyl)-2-pyridyl]-1H-pyrazol-4-yl}propoxy)-3-methoxyphenyl]acetic acid (Example 212);
(2-{3-[1-(5-chloro-2-pyridyl)-3-(1-ethylpropyl)-1H-pyrazol-4-yl]propoxy}-3-methoxyphenyl)acetic acid (Example 223);
[3-ethyl-2-(3-{3-isopropyl-1-[6-(trifluoromethyl)pyridazin-3-yl]-1H-pyrazol-4-yl}propoxy)phenyl]acetic acid (Example 245);
[2-(3-{3-isopropyl-1-[6-(trifluoromethyl)pyridazin-3-yl]-1H-pyrazol-4-yl}propoxy)-3-methoxyphenyl]acetic acid (Example 274);
[3-(3-{3-isopropyl-1-[5-(trifluoromethyl)-2-pyridinyl]-1H-pyrazol-4-yl}propoxy)-1-methyl-1H-pyrazol-4-yl]acetic acid (Example 299);
[1-ethyl-5-(3-{3-isopropyl-1-[5-(trifluoromethyl)-2-pyridinyl]-1H-pyrazol-4-yl}propoxy)-1H-pyrazol-4-yl]acetic acid (Example 322);
[1-ethyl-5-(3-{3-propyl-1-[5-(trifluoromethyl)-2-pyridinyl]-1H-pyrazol-4-yl}propoxy)-1H-pyrazol-4-yl]acetic acid (Example 326);
(2-{3-[1-(5-bromo-2-pyridinyl)-3-(1-ethylpropyl)-1H-pyrazol-4-yl]propoxy}-3-methoxyphenyl)acetic acid (Example 351); or
[2-(3-{3-tert-butyl-1-[6-(trifluoromethyl)pyridazin-3-yl]-1H-pyrazol-4-yl}propoxy)-3-methylphenyl]acetic acid (Example 367).

The salt of a compound of the formula (I), (Ia), or (Ib) (hereinafter also referred to as Compound (I)) is preferably a pharmacologically acceptable salt, and is exemplified by salts with inorganic bases, salts with organic bases, salts with inorganic acids, salts with organic acids, and salts with basic or acidic amino acids.

Preferable examples of the salts with inorganic bases include alkali metal salts such as sodium salts, potassium salts and lithium salts; alkaline earth metal salts such as calcium salts and magnesium salts; and aluminum salts and ammonium salts.

Preferable examples of the salts with organic bases include salts with trimethylamine, triethylamine, pyridine, picoline, ethanolamine, diethanolamine, triethanolamine, dicyclohexylamine, N,N-dibenzylethylenediamine, etc.

Preferable examples of the salts with inorganic acids include salts with hydrochloric acid, hydrobromic acid, nitric acid, sulfuric acid, phosphoric acid, etc.

Preferable examples of the salts with organic acids include salts with formic acid, acetic acid, trifluoroacetic acid, fumaric acid, oxalic acid, tartaric acid, maleic acid, citric acid, succinic acid, malic acid, methanesulfonic acid, benzenesulfonic acid, p-toluenesulfonic acid, etc.

Preferable examples of the salts with basic amino acids include salts with arginine, lysine, ornithine, etc.

Examples of preferable salts with acidic amino acids include salts with aspartic acid, glutamic acid, etc.

A prodrug of Compound (I) refers to a compound capable of being converted to Compound (I) by reactions of an enzyme, gastric juice, or the like, under physiological conditions in vivo, specifically a compound capable of being converted to Compound (I) upon enzymatic oxidation, reduction, hydrolysis, or the like, or a compound capable of being converted to Compound (I) upon hydrolysis or the like by gastric juice or the like. Examples of the prodrugs of Compound (I) include compounds derived by acylation, alkylation or phosphorylation of the amino group of Compound (I) (e.g., compounds derived by eicosanoylation, alanylation, pentylaminocarbonylation, (5-methyl-2-oxo-1,3-dioxolen-4-yl)methoxycarbonylation, tetrahydrofuranylation, tetrahydropyranylation, pyrrolidylmethylation, pivaloyloxymethylation or tert-butylation of the amino group of Compound (I)); compounds derived by acylation, alkylation, phosphorylation or boration of the hydroxyl group of Compound (I) (e.g., compounds derived by acetylation, palmitoylation, propanoylation, pivaloylation, succinylation, fumarylation, alanylation, dimethylaminomethylcarbonylation or tetrahydropyranylation of the hydroxyl group of Compound (I)); and compounds derived by esterification or amidation of the carboxyl group of Compound (I) (e.g., compounds derived by ethyl esterification, phenyl esterification, carboxymethyl esterification, dimethylaminomethyl esterification, pivaloyloxymethyl esterification, ethoxycarbonyloxyethyl esterification, phthalidyl esterification, (5-methyl-2-oxo-1,3-dioxolen-4-yl)methyl esterification, cyclohexyloxycarbonylethyl esterification, or methylamidation of the carboxyl group of Compound (I)). These compounds can be produced from Compound (I) by methods known per se.

The prodrug of Compound (I) may be one capable of being converted to Compound (I) under physiological conditions, as described in “Iyakuhin No Kaihatsu (Development of Drugs)”, vol. 7, Molecular Designing, published by Hirokawa Shoten, 1990, pages 163-198.

In addition, Compound (I) may be labeled with an isotope (e.g., ³H, ¹⁴C, ³⁵S, ¹²⁵I).

Furthermore, Compound (I) may be anhydrides or hydrates.

Compounds (I) and salts thereof (hereinafter also referred to as “compound of the present invention”) are of low toxicity and can be used as an agent for the prophylaxis or treatment of the various diseases mentioned below in mammals (e.g., humans, mice, rats, rabbits, dogs, cats, bovines, horses, swine, monkeys), as such or in the form of pharmaceutical compositions prepared by admixing with a pharmacologically acceptable carrier, etc.

Here, the pharmacologically acceptable carriers are exemplified by various organic or inorganic carrier substances in common use as materials for pharmaceutical preparations, and they are formulated as excipients, lubricants, binders, and disintegrants for solid preparations; and as solvents, solubilizers, suspending agents, isotonizing agents, buffers, soothing agents, etc. for liquid preparations. In addition, other additives for pharmaceutical preparations, such as antiseptics, antioxidants, coloring agents, and sweetening agents, may also be used as necessary.

Preferable examples of the excipients include lactose, saccharose, D-mannitol, D-sorbitol, starch, gelatinized starch, dextrin, crystalline cellulose, low-substituted hydroxypropylcellulose, carboxymethylcellulose sodium, gum arabic, dextrin, pullulan, light silicic anhydride, synthetic aluminum silicate, and magnesium metasilicate aluminate.

Preferable examples of the lubricants include magnesium stearate, calcium stearate, talc, and colloidal silica.

Preferable examples of the binders include gelatinized starch, sucrose, gelatin, gum arabic, methylcellulose, carboxymethylcellulose, carboxymethylcellulose sodium, crystalline cellulose, saccharose, D-mannitol, trehalose, dextrin, pullulan, hydroxypropylcellulose, hydroxypropylmethylcellulose, and polyvinylpyrrolidone.

Preferable examples of the disintegrants include lactose, saccharose, starch, carboxymethylcellulose, carboxymethylcellulose calcium, croscarmellose sodium, carboxymethyl starch sodium, light silicic anhydride, and low-substituted hydroxypropylcellulose.

Preferable examples of the solvents include water for injection, physiological saline, Ringer's solution, alcohol, propylene glycol, polyethylene glycol, sesame oil, corn oil, olive oil, and cottonseed oil.

Preferable examples of the solubilizers include polyethylene glycol, propylene glycol, D-mannitol, trehalose, benzyl benzoate, ethanol, trisaminomethane, cholesterol, triethanolamine, sodium carbonate, sodium citrate, sodium salicylate, and sodium acetate.

Preferable examples of the suspending agents include surfactants such as stearyltriethanolamine, sodium lauryl sulfate, laurylaminopropionic acid, lecithin, benzalkonium chloride, benzethonium chloride, and monostearic glycerol; hydrophilic polymers such as polyvinyl alcohol, polyvinylpyrrolidone, carboxymethylcellulose sodium, methylcellulose, hydroxymethylcellulose, hydroxyethylcellulose, and hydroxypropylcellulose; and polysorbates and polyoxyethylene-hardened castor oil.

Preferable examples of the isotonizing agents include sodium chloride, glycerol, D-mannitol, D-sorbitol, and glucose.

Preferable examples of the buffers include buffer solutions of phosphates, acetates, carbonates, citrates etc.

Preferable examples of the soothing agents include benzyl alcohol.

Preferable examples of the antiseptics include p-oxybenzoic acid esters, chlorobutanol, benzyl alcohol, phenethyl alcohol, dehydroacetic acid, and sorbic acid.

Preferable examples of the antioxidants include sulfites and ascorbates.

Preferable examples of the coloring agents include food colors such as water-soluble tar colors for food (e.g., Food Color Red Nos. 2 and 3, Food Color Yellow Nos. 4 and 5, Food Color Blue Nos. 1 and 2), water-insoluble lake colors (e.g., aluminum salts of the aforementioned water-soluble tar colors for food), and natural colors (e.g., β-carotene, chlorophyll, red oxide).

Preferable examples of the sweetening agents include saccharin sodium, dipotassium glycyrrhetinate, aspartame, and stevia.

Examples of the dosage forms of the pharmaceutical composition include oral preparations such as tablets (including sublingual tablet, orally disintegrating tablet), capsules (including soft capsules and microcapsules), powders, granules, troche, syrups; and non-oral preparations such as injections (e.g., subcutaneous injections, intravenous injections, intramuscular injections, intraperitoneal injections, drip infusions), external preparations (e.g., dermal preparations, ointments), suppositories (e.g., rectal suppositories, vaginal suppositories), pellets, preparations for nasal administration, preparations for transpulmonary administration (inhalant) and eye drop. These preparations may be controlled-release preparations (e.g., sustained-release microcapsule) such as rapid release preparations, sustained-release preparations and the like.

The pharmaceutical composition can be prepared by conventional methods in the fields of pharmaceutical manufacturing techniques, for example, methods described in the Japanese Pharmacopoeia. Specific production methods for oral preparations and non-oral preparations are hereinafter described in detail.

An oral preparation, for instance, is produced by adding to the active ingredient an excipient (e.g., lactose, saccharose, starch, D-mannitol), a disintegrant (e.g., carboxymethylcellulose calcium), a binder (e.g., gelatinized starch, gum arabic, carboxymethylcellulose, hydroxypropylcellulose, polyvinylpyrrolidone) or a lubricant (e.g., talc, magnesium stearate, polyethyleneglycol 6000), compression molding the obtained mixture, then, if necessary coating by a method known per se using a coating base for the purpose of taste masking, enteric coating or sustained release.

Examples of the coating base include a sugar coating base, a water-soluble film coating base, an enteric film coating base, a sustained-release film coating base.

As the sugar coating base saccharose is employed. Further, one or two or more species selected from talc, precipitated calcium carbonate, gelatin, gum arabic, pullulan, carnauba wax and the like may be used in combination.

Examples of the water-soluble film coating base include cellulose polymers such as hydroxypropylcellulose, hydroxypropylmethylcellulose, hydroxyethylcellulose, methylhydroxyethylcellulose; synthetic polymers such as polyvinylacetal diethylaminoacetate, aminoalkyl methacrylate copolymer E [Eudragit E (trademark), Rhom Pharma] and polyvinylpyrrolidone; polysaccharides such as pullulan.

Examples of the enteric film coating base include cellulose polymers such as hydroxypropylmethylcellulose phthalate, hydroxypropylmethylcellulose acetate succinate, carboxymethylethylcellulose, cellulose acetate phthalate; acrylic acid polymers such as methacrylic acid copolymer L [Eudragit L (trademark), Rhom Pharma], methacrylic acid copolymer LD [Eudragit L-30D55 (trademark), Rhom Pharma], methacrylic acid copolymer S [Eudragit S (trademark), Rhom Pharma]; natural products such as shellac and the like.

Examples of the sustained-release film coating base include cellulose polymers such as ethylcellulose; acrylic acid polymers such as aminoalkyl methacrylate copolymer RS [Eudragit RS (trademark), Rhom Pharma] and an ethyl acrylate-methyl methacrylate copolymer suspension [Eudragit NE (trademark), Rhom Pharma].

Two or more of the above coating bases may be used in admixture in an appropriate ratio. On the occasion of coating, a shading agent such as titanium oxide, red ferric oxide may be used.

Injections are produced by dissolving, suspending or emulsifying the active ingredient in an aqueous solvent (e.g. distilled water, physiological saline, Ringer's solution) or an oleaginous solvent (e.g. vegetable oils such as olive oil, sesame oil, cotton seed oil, corn oil; propylene glycol), together with a dispersant (e.g. polysorbate 80, polyoxyethylene-hardened castor oil 60), polyethylene glycol, carboxymethylcellulose, sodium alginate), a preservative (e.g. methylparaben, propylparaben, benzyl alcohol, chlorobutanol, phenol), an isotonizing agent (e.g. sodium chloride, glycerol, D-mannitol, D-sorbitol, glucose) and the like. If desirable, additives such as a solubilizer (e.g. sodium salicylate, sodium acetate), a stabilizer (e.g. human serum albumin), a soothing agent (e.g. benzyl alcohol), may be used.

The compound of the present invention has a hypoglycemic action, a hypolipidemic action, a hypoinsulinemic action, an insulin resistance improving action, an insulin sensitivity enhancing action, and a retinoid-related receptor function regulating action.

The term “function regulating action” used here stands for both an agonistic action and an antagonistic action.

The term “retinoid-related receptor” used here is classified as nuclear receptors, and is a DNA-binding transcription factor whose ligand is a signal molecule such as oil-soluble vitamins, etc., and may be any of a monomer receptor, a homodimer receptor and a heterodimer receptor.

Here, examples of the monomer receptor include retinoid O receptor (hereinafter, also abbreviated as ROR) α (GenBank Accession No. L14611), RORβ (GenBank Accession No. L14160), RORγ (GenBank Accession No. U16997); Rev-erb α (GenBank Accession No. M24898), Rev-erb β (GenBank Accession No. L31785); ERRα (GenBank Accession No. X51416), ERRβ (GenBank Accession No. X51417); Ftz-FIα (GenBank Accession No. S65876), Ftz-FIβ (GenBank Accession No. M81385); TIx (GenBank Accession No. S77482); GCNF (GenBank Accession No. U14666).

Examples of the homodimer receptor include homodimers formed by retinoid X receptor (hereinafter, also abbreviated as RX R) α (GenBank Accession No. X52733), RXRβ (GenBank Accession No. M84820), RXRγ (GenBank Accession No. U38480); COUPα (GenBank Accession No. X12795), COUPβ (GenBank Accession No. M64497), COUPγ (GenBank Accession No. X12794); TR2α (GenBank Accession No. M29960), TR2β (GenBank Accession No. L27586); or HNF4α (GenBank Accession No. X76930), HNF4γ (GenBank Accession No. Z49826), etc.

Examples of the heterodimer receptor include heterodimers which are formed by the above-mentioned retinoid X receptor (RXRα, RXRβ or RXTγ) and one receptor selected from retinoid A receptor (hereinafter, also abbreviated as RAR) α (GenBank Accession No. X06614), RARβ (GenBank Accession No. Y00291), RARγ (GenBank Accession No. M24857); thyroid hormone receptor (hereinafter, also abbreviated as TR) α (GenBank Accession No. M24748), TRβ (GenBank Accession No. M26747); vitamin D receptor (VDR) (GenBank Accession No. J03258): peroxisome proliferator-activated receptor (hereinafter, also abbreviated as PPAR) α (GenBank Accession No. L02932), PPARβ (PPARδ) (GenBank Accession No. U10375), PPAR γ (GenBank Accession No. L40904); LXRα (GenBank Accession No. U22662), LXRβ (GenBank Accession No. U14534); FXR (GenBank Accession No. U18374); MB67 (GenBank Accession No. L29263); ONR (GenBank Accession No. X75163); and NURα (GenBank Accession No. L13740), NURβ (GenBank Accession No. X75918) and NURγ (GenBank Accession No. U12767).

The compound of the present invention has an excellent ligand activity particularly to retinoid X receptors (RXRα, RXRβ, RXRγ) and to peroxisome proliferator-activated receptors (PPARα, PPARβ (PPARδ), PPARγ) among the above-mentioned retinoid-related receptors. It is useful as an agonist, a partial agonist, an antagonist or a partial antagonist to these receptors.

Further, the compound of the present invention has an excellent ligand activity to peroxisome proliferator-activated receptors in heterodimer receptors formed from a retinoid X receptor and a peroxisome proliferator-activated receptor (e.g. heterodimer receptors formed from RXRα and PPARδ, heterodimer receptors formed from RXRα and PPARγ).

Accordingly, the retinoid-related receptor ligand of the present invention can be used advantageously as a peroxisome proliferator-activated receptor ligand or a retinoid X receptor ligand.

The compound of the present invention can be used as, for example, an agent for the prophylaxis or treatment of diabetes (e.g., type 1 diabetes, type 2 diabetes, gestational diabetes); an agent for the prophylaxis or treatment of hyperlipidemia (e.g., hypertriglyceridemia, hypercholesterolemia, hypo-high-density-lipoproteinemia, postprandial hyperlipemia); an agent for improving insulin resistance; an agent for enhancing insulin sensitivity; an agent for the prophylaxis or treatment of impaired glucose tolerance (IGT); and an agent for preventing progress from impaired glucose tolerance to diabetes.

Regarding diagnostic criteria of diabetes, new diagnostic criteria were reported by the Japan Diabetes Society in 1999.

According to this report, diabetes is a condition wherein the fasting blood glucose level (glucose concentration in venous plasma) is not less than 126 mg/dl, the 2-hour value (glucose concentration in venous plasma) of the 75 g oral glucose tolerance test (75 g OGTT) is not less than 200 mg/dl, or the non-fasting blood glucose level (glucose concentration in venous plasma) is not less than 200 mg/dl. In addition, a condition which does not fall within the scope of the above definition of diabetes, and which is not a “condition wherein the fasting blood glucose level (glucose concentration in venous plasma) is less than 110 mg/dl or the 2-hour value (glucose concentration in venous plasma) of the 75 g oral glucose tolerance test (75 g OGTT) is less than 140 mg/dl” (normal type), is called the “borderline type”.

In addition, regarding diagnostic criteria for diabetes, new diagnostic criteria were reported by ADA (American Diabetic Association) in 1997 and by WHO in 1998.

According to these reports, diabetes is a condition wherein the fasting blood glucose level (glucose concentration in venous plasma) is not less than 126 mg/dl, and the 2-hour value (glucose concentration in venous plasma) of the 75 g oral glucose tolerance test is not less than 200 mg/dl.

In addition, according to the above reports, impaired glucose tolerance is a condition wherein the fasting blood glucose level (glucose concentration in venous plasma) is less than 126 mg/dl, and the 2-hour value (glucose concentration in venous plasma) of the 75 g oral glucose tolerance test is not less than 140 mg/dl and less than 200 mg/dl. Furthermore, according to the ADA report, a condition wherein the fasting blood glucose level (glucose concentration in venous plasma) is not less than 110 mg/dl and less than 126 mg/dl, is called IFG (impaired fasting glucose). On the other hand, according to the WHO report, a condition of IFG (impaired fasting glucose) as such wherein the 2-hour value (glucose concentration in venous plasma) of the 75 g oral glucose tolerance test is less than 140 mg/dl, is called IFG (impaired fasting glycemia).

The compound of the present invention can be used as an agent for the prophylaxis or treatment of diabetes, borderline type, impaired glucose tolerance, IFG (impaired fasting glucose) and IFG (impaired fasting glycemia) as defined by the above new diagnostic criteria. Furthermore, the compound of the present invention can also be used to prevent the progression of the borderline type, impaired glucose tolerance, IFG (impaired fasting glucose) or IFG (impaired fasting glycemia) to diabetes.

The compound of the present invention possesses a total cholesterol lowering action and enhance a plasma anti-arteriosclerosis index [(HDL cholesterol/total cholesterol)×100], and therefore, can be used as an agent for the prophylaxis or treatment of arteriosclerosis (e.g., atherosclerosis), and the like. Particularly, since the compound of the present invention concurrently has a hypoglycemic action and a total cholesterol lowering action, it is extremely useful as an agent for the prophylaxis or treatment of arteriosclerosis in diabetic patients.

The compound of the present invention can be used also as an agent for the prophylaxis or treatment of diabetic complications (e.g., neuropathy, nephropathy, retinopathy, cataract, macroangiopathy, osteopenia, diabetic hyperosmolar coma, infectious diseases (e.g., respiratory infection, urinary tract infection, gastrointestinal tract infection, dermal soft tissue infection, inferior limb infection), diabetic gangrene, xerostomia, lowered sense of hearing, cerebrovascular disease, peripheral circulatory disturbance, etc.), obesity, osteoporosis, cachexia (e.g., carcinomatous cachexia, tuberculous cachexia, diabetic cachexia, hemopathic cachexia, endocrinopathic cachexia, infectious cachexia, cachexia induced by acquired immunodeficiency syndrome), fatty liver, hypertension, polycystic ovary syndrome, renal diseases (e.g., diabetic nephropathy, glomerular nephritis, glomerulosclerosis, nephrotic syndrome, hypertensive nephrosclerosis, terminal renal disorder), muscular dystrophy, myocardiac infarction, angina pectoris, cerebrovascular disease (e.g., cerebral infarction, cerebral apoplexy), insulin resistance syndrome, syndrome X, hyperinsulinemia, hyperinsulinemia-induced sensory disorder, tumor (e.g., leukemia, breast cancer, prostate cancer, skin cancer), irritable intestinum syndrome, acute or chronic diarrhea, inflammatory diseases (e.g., Alzheimer's disease, chronic rheumatoid arthritis, spondylitis deformans, osteoarthritis, lumbago, gout, postoperative or traumatic inflammation, remission of swelling, neuralgia, pharyngolaryngitis, cystitis, hepatitis (including steatohepatitis such as non-alcoholic steatohepatitis), pneumonia, pancreatitis, inflammatory colitis, ulcerative colitis), visceral obesity syndrome, and the like.

The compound of the present invention can be used for ameliorating bellyache, nausea, vomiting, or dysphoria in epigastrium, each of which is accompanied by gastrointestinal ulcer, acute or chronic gastritis, biliary dyskinesia, or cholecystitis.

The compound of the present invention can control (enhance or inhibit) appetite and food intake, and therefore, can be used as an agent for treating leanness and cibophobia (the weight increase in administration subjects suffering from leanness or cibophobia) or an agent for treating obesity.

Since the compound of the present invention has a TNF-α suppressing effect (a TNF-α production amount-lowering effect and a TNF-α activity lowering effect in tissues of living organisms), the compound of the present invention can be also used as an agent for the prophylaxis or treatment of TNF-α mediated inflammatory diseases. Examples of such inflammatory diseases include diabetic complications (e.g., retinopathy, nephropathy, neuropathy, macroangiopathy), rheumatoid arthritis, spondylitis deformans, osteoarthritis, lumbago, gout, postoperative or traumatic inflammation, remission of swelling, neuralgia, pharyngolaryngitis, cystitis, hepatitis, pneumonia, gastric mucosal injury (including aspirin-induced gastric mucosal injury), and the like.

The compound of the present invention has an apoptosis inhibitory activity, and can be used as an agent for the prophylaxis or treatment of diseases mediated by promotion of apoptosis. Examples of the diseases mediated by promotion of apoptosis include viral diseases (e.g., AIDS, fulminant hepatitis), neurodegenerative diseases (e.g., Alzheimer's disease, Parkinson's disease, amyotropic lateral sclerosis, retinitis pigmentosa, cerebellar degeneration), myelodysplasia (e.g., aplastic anemia), ischemic diseases (e.g., myocardial infarction, cerebral apoplexy), hepatic diseases (e.g., alcoholic hepatitis, hepatitis B, hepatitis C), joint-diseases (e.g., osteoarthritis), atherosclerosis, and the like.

The compound of the present invention can be used for reducing visceral fats, inhibiting accumulation of visceral fats, ameliorating glycometabolism, ameliorating lipidmetabolism, ameliorating insulin resistance, inhibiting production of oxidized LDL, ameliorating lipoprotein metabolism, ameliorating coronary artery metabolism, preventing or treating cardiovascular complications, preventing or treating heart failure complications, lowering blood remnant, preventing or treating anovulation, preventing or treating hirsutism, preventing or treating hyperandrogenism, and the like.

The compound of the present invention can be used for secondary prevention and for inhibition in progress, of the various diseases described above (e.g., cardiovascular events such as myocardial infarction, etc.).

The compound of the present invention has a GPR40 receptor function modulating activity (agonistic activity and antagonistic activity; preferably agonistic activity), namely, an action to change the bindability between fatty acid, which is a ligand of GPR40 receptor, and a GPR40 receptor, and is used as a modulator of physiological function, in which GPR40 receptor is involved, or a prophylactic or therapeutic agent of a disease state or a disease, in which GPR40 receptor is involved.

As used herein, as the “modulator of physiological function, in which GPR40 receptor is involved”, for example, insulin secretion modulator (preferably insulin secretagogue), pancreatic β cells protective agent and the like can be mentioned. As the “disease state or a disease, in which GPR40 receptor is involved”, for example, diabetes (e.g., type 1 diabetes, type 2 diabetes), impaired glucose tolerance (IGT), ketosis, acidosis, diabetic neuropathy, diabetic nephropathy, diabetic retinopathy, hyperlipidemia, genital disorder, dermatosis, arthropathy, osteopenia, arteriosclerosis, thrombotic disease, dyspepsia, memory and learning disorder, obesity, hypoglycemia, hypertension, edema, insulin resistance, unstable diabetes, fatty atrophy, insulin allergy, insulinoma, lipotoxicity, cancer and the like can be mentioned.

Although the dose of the compound of the present invention varies depending on administration subject, administration route, target disease, clinical condition, etc., it is, for instance, about 0.005 to 50 mg/kg body weight, preferably 0.01 to 2 mg/kg body weight, more preferably 0.025 to 0.5 mg/kg body weight, as a usual dosage per administration for oral administration to an adult diabetic patient. This dose is desirably administered 1 to 3 times a day.

The compound of the present invention can be used in combination with a drug such as a therapeutic agent for diabetes, a therapeutic agent for diabetic complications, an antihyperlipidemic agent, a hypotensive agent, an antiobesity agent, a diuretic agent, a chemotherapeutic agent, an immunotherapeutic agent, antithrombotic agent, ameliorative agent for cachexia, and the like (hereinafter abbreviated as a combination drug). The combination drug may be a low molecular weight compound or a high molecular weight protein, polypeptide, antibody, vaccine and the like. On such occasions, the timing of administration of the compound of the present invention and that of the combination drug is not limited. They may be administered simultaneously or at staggered times to the administration subject. Moreover, the compound of the present invention and a combination drug may be administered as two kinds of preparations respectively containing an active ingredient, or as a single preparation containing both active ingredients.

The dose of the combination drug can be appropriately selected based on the dose which is clinically employed. The proportion of the compound of the present invention and the combination drug can be appropriately selected according to the administration subject, administration route, target disease, clinical condition, combination, and other factors. In cases where the administration subject is human, for instance, the combination drug may be used in an amount of 0.01 to 100 parts by weight per part by weight of the compound of the present invention.

Examples of the therapeutic agent for diabetes include insulin preparations (e.g., animal insulin preparations extracted from the bovine or swine pancreas; human insulin preparations synthesized by a genetic engineering technique using Escherichia coli or a yeast, insulin zinc; protamine zinc insulin; fragment or derivative of insulin (e.g., INS-1 and the like)), insulin resistance improving agents (e.g., pioglitazone hydrochloride, troglitazone, rosiglitazone or its maleate, GI-262570, Reglixane (JTT-501), Netoglitazone (MCC-555), YM-440, KRP-297, CS-011, FK-614, compounds described in WO99/58510 (e.g., (E)-4-[4-(5-methyl-2-phenyl-4-oxazolylmethoxy)benzyloxyimino]-4-phenylbutyric acid), Tesaglitazar (AZ-242), Ragaglitazar (NN-622), BMS-298585, ONO-5816, BM-13-1258, LM-4156, MBX-102, LY-519818, MX-6054, LY-510929 and the like), α-glucosidase inhibitors (e.g., voglibose, acarbose, miglitol, emiglitate), biguanides (e.g., phenformin, metformin, buformin), insulin secretagogues [sulfonylureas (e.g., tolbutamide, glibenclamide, gliclazide, chlorpropamide, tolazamide, acetohexamide, glyclopyramide, glimepiride, glipizide, glybuzole), repaglinide, nateglinide, mitiglinide or its calcium salt hydrate, GLP-1), dipeptidylpeptidase IV inhibitors (e.g., NVP-DPP-278, PT-100, P32/98, LAF237), β3 agonists (e.g., CL-316243, SR-58611-A, UL-TG-307, SB-226552, AJ-9677, BMS-196085, AZ40140), amyrin agonist (e.g., pramlintide), phosphotyrosine phosphatase inhibitors (e.g., vanadic acid), gluconeogenesis inhibitors (e.g., glycogen phosphorylase inhibitors, glucose-6-phosphatase inhibitors, glucagon antagonists), SGLUT (sodium-glucose cotransporter) inhibitors (e.g., T-1095).

Examples of the therapeutic agent for diabetic complications include aldose reductase inhibitors (e.g., tolrestat, epalrestat, zenarestat, zopolrestat, minalrestat, fidarestat (SNK-860), CT-112), neurotrophic factors (e.g., NGF, NT-3, BDNF), neurotrophic factor production-secretion promoter [e.g., neurotrophin production secretion promoter (e.g., 4-(4-chlorophenyl)-2-(2-methyl-1-imidazole)-5-(3-(2-methylphenoxy)propyl)oxazole and the like) described in WO01/14372], PKC inhibitors (e.g., LY-333531), AGE inhibitors (e.g., ALT946, pimagedine, pyratoxathine, N-phenacylthiazolium bromide (ALT766), EXO-226), active oxygen scavengers (e.g. thioctic acid), cerebral vasodilators (e.g., tiapuride, mexiletine).

Examples of the antihyperlipidemic agent include HMG-CoA reductase inhibitors (e.g., pravastatin, simvastatin, lovastatin, atorvastatin, fluvastatin, lipantil, cerivastatin, itavastatin, ZD-4522 or their salts (e.g., sodium salt)), fibrate compounds (e.g., bezafibrate, beclofibrate, binifibrate, cyprofibrate, clinofibrate, clofibrate, clofibric acid, etofibrate, fenofibrate, gemfibrozil, nicofibrate, pirifibrate, ronifibrate, simfibrate, theofibrate), squalene synthase inhibitors (e.g., compound described in WO97/10224, such as N-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]piperidine-4-acetic acid and the like), ACAT inhibitors (e.g., Avasimibe, Eflucimibe), anion exchange resins (e.g., cholestylamine), probuchol, nicotinic pharmaceutical agents (e.g., nicomol, niceritrol), ethyl icosapentate, phytosterol (e.g., soysterol, γ-oryzanol) and the like.

Examples of the hypotensive agent include angiotensin converting enzyme inhibitors (e.g., captopril, enalapril, delapril), angiotensin II antagonists (e.g., candesartan cilexetil, losartan, eprosartan, valsartan, termisartan, irbesartan, tasosartan), calcium antagonist (e.g., manidipine, nifedipine, nicardipine, amlodipine, efonidipine), potassium channel opener (e.g., levcromakalim, L-27152, AL 0671 NIP-121) and clonidine.

Examples of the antiobesity agent include antiobesity drugs acting on the central nervous system (e.g. dexfenfluramine, fenfluramine, phentermine, sibutramine, anfepramon, dexamphetamine, mazindol, phenylpropanolamine, clobenzorex; MCH receptor antagonists (e.g., SB-568849; SNAP-7941; compounds described in WO01/82925 and WO01/87834), pancreatic lipase inhibitors (e.g. orlistat), β3 agonists (e.g. CL-316243, SR-58611-A, UL-TG-307, SB-226552, AJ-9677, BMS-196085, AZ-40140), anorectic peptides (e.g. leptin, CNTF (Ciliary Neurotrophic Factor)), cholecystokinin agonists (e.g. lintitript, FPL-15849).

Examples of the diuretic agent include xanthine derivatives (e.g., theobromine and sodium salicylate, theobromine and calcium salicylate), thiazide preparations (e.g., ethiazide, cyclopenthiazide, trichlormethiazide, hydrochlorothiazide, hydroflumethiazide, benzylhydrochlorothiazide, penflutizide, polythiazide, methyclothiazide), antialdosterone preparations (e.g., spironolactone, triamterene), carbonate dehydratase inhibitors (e.g., acetazolamide), chlorobenzenesulfonamide preparations (e.g., chlorthalidone, mefruside, indapamide), azosemide, isosorbide, ethacrynic acid, piretanide, bumetanide, furosemide.

Examples of the chemotherapeutic agent include alkylating agents (e.g., cyclophosphamide, ifosamide), metabolic antagonists (e.g., methotrexate, 5-fluorouracil or derivative thereof), antitumor antibiotics (e.g., mitomycin, adriamycin), plant-derived antitumor agents (e.g., vincristine, vindesine, Taxol), cisplatin, carboplatin, etoposide. Among these, 5-fluorouracil derivatives such as Furtulon and Neo-Furtulon are preferable.

Examples of the immunotherapeutic agent include microorganism- or bacterium-derived components (e.g., muramyl dipeptide derivatives, Picibanil), immunopotentiator polysaccharides (e.g., lentinan, schizophyllan, krestin), genetically engineered cytokines (e.g., interferons, interleukins (IL)), colony stimulating agents (e.g., granulocyte colony stimulating factor, erythropoietin), etc. Among these, interleukins such as IL-1, IL-2, IL-12 and the like are preferable.

As the antithrombotic agent, for example, heparin (e.g., heparin sodium, heparin calcium, dalteparin sodium), warfarin (e.g., warfarin potassium), antithrombin agents (e.g., aragatroban), thrombolytic agents (e.g., urokinase, tisokinase, alteplase, nateplase, monteplase, pamiteplase), platelet aggregation inhibitors (e.g., ticlopidine hydrochloride, cilostazol, ethyl icosapentate, beraprost sodium, sarpogrelate hydrochloride) and the like can be mentioned.

Examples of the ameliorative agent for cachexia include cyclooxygenase inhibitors (e.g., indomethacin) (Cancer Research, vol. 49, pp. 5935-5939, 1989), progesterone derivatives (e.g., megestrol acetate) (Journal of Clinical Oncology, vol. 12, pp. 213-225, 1994), glucocorticoids (e.g. dexamethasone), metoclopramide pharmaceuticals, tetrahydrocannabinol pharmaceuticals (the above references are applied to both), fat metabolism ameliorating agents (e.g., eicosapentanoic acid) (British Journal of Cancer, vol. 68, pp. 314-318, 1993), growth hormones, IGF-1, and antibodies to the cachexia-inducing factor TNF-α, LIF, IL-6 or oncostatin M. As the combination drug, nerve regeneration promoting drugs (e.g., Y-128, VX-853, prosaptide), antidepressants (e.g., desipramine, amitriptyline, imipramine), anticonvulsants (e.g., lamotrigine), antiarrhythmic drugs (e.g., mexiletine), acetylcholine receptor ligands (e.g., ABT-594), endothelin receptor antagonists (e.g., ABT-627), monoamine uptake inhibitors (e.g., tramadol), narcotic analgesics (e.g., morphine), GABA receptor agonists (e.g., gabapentine), a2 receptor agonists (e.g., clonidine), local analgesics (e.g., capsaicin), protein kinase C inhibitors (e.g., LY-333531), antianxiety drugs (e.g., benzodiazepine), phosphodiesterase inhibitors (e.g., sildenafil (citrate)), dopamine agonists (e.g., apomorphine), osteoporosis therapeutic agents (e.g., alphacalcidol, calcitriol, elcatonin, salmon calcitonine, estriol, ipriflavone, pamidronate disodium, arendronate disodium hydrate, incadronate disodium), antidementia drugs (e.g., tacrine, donepezil, rivastigmine, galantamine), therapeutic agents for anischuria or polakisuria (e.g., flavoxate hydrochloride, oxybutynin hydrochloride, propiverine hydrochloride), midazolam, ketoconazole and the like can be mentioned.

The combination drug is preferably an insulin preparation, an insulin resistance improving agent, an α-glucosidase inhibitor, a biguanide, an insulin secretagogue (preferably sulfonylurea), and the like.

The above combination drugs can be used as a mixture of two or more species in an appropriate ratio. In the case of using two or more combination drugs, preferable combinations include the following.

1) an insulin resistance improving agent and an insulin preparation;

2) an insulin resistance improving agent and an insulin secretagogue;

3) an insulin resistance improving agent and an α-glucosidase inhibitor;

4) an insulin resistance improving agent and a biguanide;

5) an insulin preparation and a biguanide;

6) an insulin preparation and an insulin secretagogue;

7) an insulin preparation and an α-glucosidase inhibitor;

8) an insulin secretagogue and an α-glucosidase inhibitor;

9) an insulin secretagogue and a biguanide;

10) an insulin resistance improving agent, an insulin preparation and a biguanide;

11) an insulin resistance improving agent, an insulin preparation and an insulin secretagogue;

12) an insulin resistance improving agent, an insulin preparation and an α-glucosidase inhibitor;

13) an insulin resistance improving agent, an insulin secretagogue and a biguanide;

14) an insulin resistance improving agent, an insulin secretagogue and an α-glucosidase inhibitor; and

15) an insulin resistance improving agent, a biguanide and an α-glucosidase inhibitor.

By a combined use of the compound of the present invention and a combination drug, superior effects such as potentiation of the action of the compound of the present invention and/or the combination drug (preferably insulin preparation, insulin resistance improving agent, insulin secretagogue or biguanide), reduction of the dose of the compound of the present invention and/or the combination drug (preferably insulin resistance improving agent, insulin secretagogue or biguanide), reduction of the side effect of the compound of the present invention and/or the combination drug and the like can be obtained.

The production method for the compound of the present invention is hereinafter described.

Compound (I) can be produced by a method known per se, such as METHODs A-E and METHOD K shown in the following or a method analogous thereto. In each of the following production methods, the starting material may be used in the form of a salt, and examples of such salt include those exemplified as the salts of the aforementioned compound (I).

The compound (I-1), having —O—, —S— or —NR³— (R³ is as defined above) for Xb in the formula (I), can be produced by, for example, the following METHOD A. 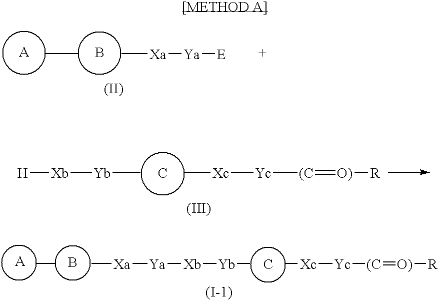
wherein E is a leaving group, and other symbols are as defined above.

As used herein, as the leaving group represented by E, for example, a hydroxy group, a halogen atom, —OSO₂R¹¹ (R¹¹ is alkyl group having 1 to 6 carbon atoms or aryl group having 6 to 10 carbon atoms which may be substituted by alkyl group having 1 to 6 carbon atoms) and the like can be mentioned.

As the halogen atom, fluorine, chlorine, bromine, iodine and the like can be mentioned. Of these, chlorine, bromine and iodine are preferable.

As the alkyl group having 1 to 6 carbon atoms of the “alkyl group having 1 to 6 carbon atoms” and “aryl group having 6 to 10 carbon atoms which may be substituted by alkyl group having 1 to 6 carbon atoms” represented by R¹¹, for example, methyl, ethyl, propyl, isopropyl, butyl, isobutyl, sec.-butyl and t.-butyl can be preferably mentioned, particularly preferably methyl.

As the aryl group having 6 to 10 carbon atoms of the “aryl group having 6 to 10 carbon atoms which may be substituted by alkyl group having 1 to 6 carbon atoms” represented by R¹¹, for example, phenyl, naphthyl can be mentioned, particularly preferably phenyl.

R¹¹ is particularly preferably methyl, tolyl and the like.

In this method, compound (II) and compound (III) are reacted to give compound (I-1).

When E is hydroxy group, this reaction is carried out according to a method known per se, such as a method described in Synthesis, page 1 (1981), or a method analogous thereto. That is, this reaction is generally carried out in the presence of an organic phosphorus compound and an electrophilic agent in a solvent which does not interfere with the reaction.

As the organic phosphorus compound, for example, triphenylphosphine, tributylphosphine and the like can be mentioned.

As the electrophilic agent, for example, diethyl azodicarboxylate, diisopropyl azodicarboxylate, azodicarbonyldipiperazine and the like can be mentioned.

The amount of the organic phosphorus compound and electrophilic agent to be used is preferably about 1-about 5 molar equivalents relative to compound (III).

As the solvent which does not interfere with the reaction, for example, ethers such as diethyl ether, tetrahydrofuran, dioxane and the like; halogenated hydrocarbons such as chloroform, dichloromethane and the like; aromatic hydrocarbons such as benzene, toluene, xylene and the like; amides such as N,N-dimethylformamide and the like; sulfoxides such as dimethyl sulfoxide and the like, and the like can be mentioned. These solvents may be used after mixing at a suitable ratio.

The amount of the compound (II) to be used is preferably about 1-about 5 molar equivalents relative to compound (III).

The reaction temperature is generally about −50° C. to about 150° C., preferably about −10° C. to about 100° C.

The reaction time is generally about 0.5-about 20 hours.

When E is a halogen atom or —OSO₂R¹¹, this reaction is carried out according to a conventional method in the presence of a base in a solvent which does not interfere with the reaction.

As the base, for example, alkali metal salts or alkaline earth metal salts such as potassium hydroxide, sodium hydroxide, sodium hydrogen carbonate, potassium carbonate, sodium carbonate, cesium carbonate, potassium hydrogen carbonate, potassium acetate, sodium acetate, potassium propionate, sodium propionate and the like; amines such as pyridine, triethylamine, N,N-dimethylaniline, 1,8-diazabicyclo[5.4.0]undec-7-ene, trimethylamine, diisopropylethylamine, tripropylamine, N-methylmorpholine, 1,4-diazabicyclo[2.2.2]octane (DABCO), proton sponge, 4-dimethylaminopyridine, 4-diethylaminopyridine, picoline, quinoline and the like; metal hydrides such as potassium hydride, sodium hydride, calcium hydride and the like; alkaline metal alkoxides such as sodium methoxide, sodium ethoxide, potassium t.-butoxide; quaternary ammonium hydroxides (e.g., Triton B (trademark), tetrabutylammonium hydroxide) and the like can be mentioned.

The amount of these bases to be used is preferably about 1-about 5 molar equivalents relative to compound (III).

As the solvent which does not interfere with the reaction, for example, aromatic hydrocarbons such as benzene, toluene, xylene and the like; ethers such as tetrahydrofuran, dioxane, diethyl ether and the like; ketones such as acetone, 2-butanone and the like; halogenated hydrocarbons such as chloroform, dichloromethane and the like; amides such as N,N-dimethylformamide and the like; sulfoxides such as dimethyl sulfoxide and the like; and the like can be mentioned. These solvents may be used after mixing at a suitable ratio.

The amount of the compound (II) to be used is preferably about 1-about 5 molar equivalents relative to compound (III).

The reaction temperature is generally about −50° C. to about 150° C., preferably about −10° C. to about 100° C.

The reaction time is generally about 0.5-about 20 hours.

The compound (I-1) thus obtained can be isolated and purified by a known means of separation and purification, such as concentration, concentration under reduced pressure, solvent extraction, crystallization, recrystallization, phase transfer, chromatography and the like.

The compound (II) and compound (III) to be used as a starting material in the above-mentioned METHOD A can be produced by, for example, a method described in WO 01/38325 and the like, or a method analogous thereto.

The compound (I-3), having —S(O)ₘ— (m is 1 or 2) for Xb in the formula (I), can be produced by, for example, the following METHOD B. 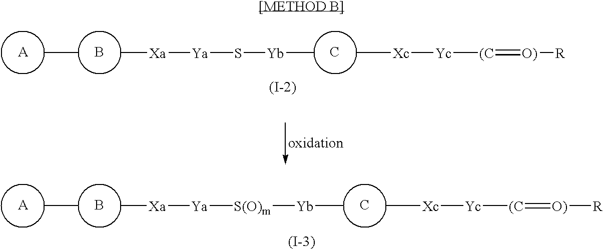
wherein the symbols in the formula are as defined above.

In this method, compound (I-2) is subjected to oxidation reaction to give compound (I-3). This reaction is generally carried out using an oxidant in a solvent which does not interfere with the reaction.

As the oxidant, for example, 3-chlorophenylperbenzoic acid, sodium periodate, hydrogen peroxide, peracetic acid and the like can be mentioned.

As the solvent which does not interfere with the reaction, for example, ethers such as diethyl ether, tetrahydrofuran, dioxane and the like; halogenated hydrocarbons such as chloroform, dichloromethane and the like; aromatic hydrocarbons such as benzene, toluene, xylene and the like; amides such as N,N-dimethylformamide and the like; alcohols such as ethanol, methanol and the like; and the like can be mentioned. These solvents may be used after mixing at a suitable ratio.

The reaction temperature is generally about −50° C. to about 150° C., preferably about −10° C. to about 100° C.

The reaction time is generally about 0.5-about 20 hours.

The compound (I-3) thus obtained can be isolated and purified by a known means of separation and purification, such as concentration, concentration under reduced pressure, solvent extraction, crystallization, recrystallization, phase transfer, chromatography and the like.

The compound (I-2) to be used as a starting material in the above-mentioned METHOD B can be produced by, for example, the above-mentioned METHOD A.

The compound (I-5), having —OH for R in the formula (I), can be also produced by, for example, the following METHOD C. 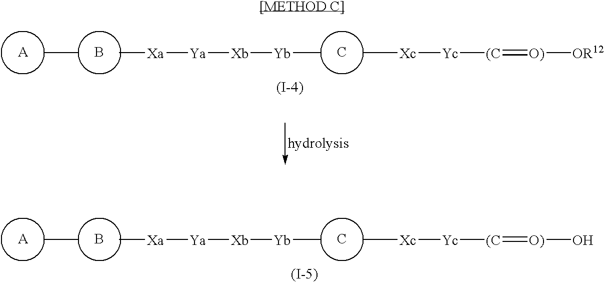
wherein R¹² is an optionally substituted hydrocarbon group, and other symbols are as defined above.

In this method, compound (I-4) is subjected to hydrolysis reaction to give compound (I-5).

As the “optionally substituted hydrocarbon group” represented by the above-mentioned R¹², those exemplified as the aforementioned R⁴ can be mentioned. R¹² is preferably an alkyl group having 1 to 6 carbon atoms, more preferably methyl, ethyl and the like.

This reaction is carried out according to a conventional method in the presence of an acid or base in an aqueous solvent.

As the acid, for example, inorganic acids such as hydrochloric acid, sulfuric acid, hydrobromic acid and the like; organic acids such as acetic acid and the like; and the like can be mentioned.

As the base, for example, alkaline metal carbonates such as potassium carbonate, sodium carbonate and the like; alkaline metal alkoxides such as sodium methoxide and the like; alkaline metal hydroxides such as potassium hydroxide, sodium hydroxide, lithium hydroxide and the like; and the like can be mentioned.

The amount of the acid or base to be used is generally an excess amount relative to compound (I-4). Preferably, the amount of the acid to be used is about 2-about 50 equivalent amount relative to compound (I-4), and the amount of the base to be used is about 1.2-about 5 equivalent amount relative to compound (I-4).

As the aqueous solvent, for example, a mixed solvent of water with one or more kinds of solvent selected from alcohols such as methanol, ethanol and the like; ethers such as tetrahydrofuran, dioxane, diethyl ether and the like; dimethyl sulfoxide, acetone and the like, and the like can be mentioned.

The reaction temperature is generally about −20° C. to about 150° C., preferably about −10° C. to about 100° C.

The reaction time is generally about 0.1-about 20 hours.

The compound (I-5) thus obtained can be isolated and purified by a known means of separation and purification, such as concentration, concentration under reduced pressure, solvent extraction, crystallization, recrystallization, phase transfer, chromatography and the like.

The compound (I-4) to be used as a starting material in the above-mentioned METHOD C can be produced by, for example, the above-mentioned METHOD A or METHOD B.

The compound (I-6), having —NR⁵R⁶ (R⁵ and R⁶ are as defined above) for R in the formula (I), can be also produced by, for example, the following METHOD D. 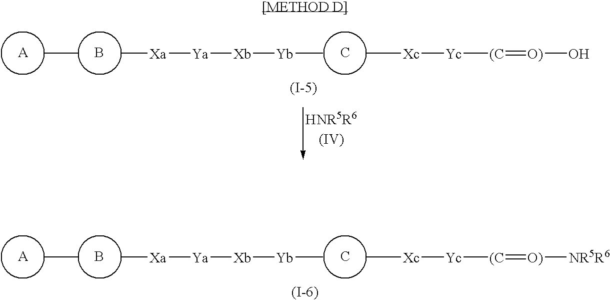
wherein the symbols in the formula are as defined above.

In this method, compound (I-5) is subjected to amidation reaction to give compound (I-6). This reaction is carried out according to a method known per se, such as a method comprising direct condensation of compound (I-5) and compound (IV) using a condensing agent, a method comprising appropriate reaction of a reactive derivative of compound (I-5) with compound (IV) and the like. As used herein, as the reactive derivative of compound (I-5), for example, acid anhydrides, acid halides (e.g., acid chlorides, acid bromides), imidazolide, or mixed acid anhydride (e.g., anhydrides with methylcarbonate, ethylcarbonate, or isobutylcarbonate) and the like can be mentioned.

As the aforementioned condensing agent, for example, generally known condensing agents such as carbodiimide condensing reagents (e.g., dicyclohexylcarbodiimide, diisopropylcarbodiimide, 1-ethyl-3-dimethylaminopropylcarbodiimide, hydrochloride thereof and the like); phosphoric acid condensing reagents (e.g., diethyl cyanophosphonate, diphenylphosphoryl azide and the like); carbonyldiimidazole, 2-chloro-1,3-dimethylimidazolium tetrafluoroborate and the like can be mentioned.

As the solvent to be used for the method using a condensing agent, for example, amides such as N,N-dimethylformamide, N,N-dimethylacetamide and the like; halogenated hydrocarbons such as chloroform, dichloromethane and the like; aromatic hydrocarbons such as benzene, toluene and the like; ethers such as tetrahydrofuran, dioxane, diethyl ether and the like; ethyl acetate, water and the like can be mentioned. These solvents may be used after mixing at a suitable ratio.

The amount of compound (IV) to be used is generally 0.1-10 molar equivalents, preferably 0.3-3 molar equivalents, relative to compound (I-5).

The amount of the condensing agent to be used is generally 0.1-10 molar equivalents, preferably 0.3-3 molar equivalents, relative to compound (I-5).

When a carbodiimide condensing reagent such as dicyclohexylcarbodiimide, diisopropylcarbodiimide, 1-ethyl-3-dimethylaminopropylcarbodiimide, hydrochloride thereof and the like is used as the condensing agent, the reaction efficiency can be improved by the use of a suitable condensation promoter (e.g., 1-hydroxy-7-azabenzotriazole, 1-hydroxybenzotriazole, N-hydroxysuccinimide, N-hydroxyphthalimide and the like) as necessary. When a phosphoric acid condensing reagent such as diethyl cyanophosphonate, diphenylphosphoryl azide and the like is used as the condensing agent, the reaction efficiency can be generally improved by the addition of an organic amine base such as triethylamine and the like.

The amount of the above-mentioned condensation promoter and organic amine base is 0.1-10 molar equivalents, preferably 0.3-3 molar equivalents, relative to compound (I-5).

The reaction temperature is generally −30° C. to 100° C.

The reaction time is generally 0.5-60 hours.

In the method using a reactive derivative of compound (I-5), for example, an acid halide is used as the reactive derivative of compound (I-5), the reaction is carried out in the presence of a base in a solvent which does not interfere with the reaction.

As the base, for example, amines such as triethylamine, N-methylmorpholine, N,N-dimethylaniline and the like; alkali metal salts such as sodium hydrogen carbonate, sodium carbonate, potassium carbonate and the like; and the like can be mentioned.

As the solvent which does not interfere with the reaction, for example, halogenated hydrocarbons such as chloroform, dichloromethane and the like; aromatic hydrocarbons such as benzene, toluene and the like; ethers such as tetrahydrofuran, dioxane, diethyl ether and the like, ethyl acetate, water and the like can be mentioned. These solvents may be used after mixing at a suitable ratio.

The amount of the compound (IV) to be used is 0.1-10 molar equivalents, preferably 0.3-3 molar equivalents, relative to compound (I-5).

The reaction temperature is generally −30° C. to 100° C.

The reaction time is generally 0.5-20 hours.

The above-mentioned acid halide can be produced using compound (I-5), for example, by a method described in J. org. Chem., vol. 52, p. 5143 (1987) and the like, or a method analogous thereto.

When a mixed acid anhydride is used as the reactive derivative of compound (I-5), moreover, compound (I-5) is reacted with a chlorocarbonic ester (e.g., methyl chlorocarbonate, ethyl chlorocarbonate, isobutyl chlorocarbonate) in the presence of a base (e.g., amines such as triethylamine, N-methylmorpholine, N,N-dimethylaniline and the like; alkali metal salt such as sodium hydrogen carbonate, sodium carbonate, potassium carbonate and the like) and then reacted with compound (IV).

The amount of compound (IV) to be used is generally 0.1-10 molar equivalents, preferably 0.3-3 molar equivalents relative to compound (I-5).

The reaction temperature is generally −30° C. to 100° C.

The reaction time is generally 0.5-20 hours.

The compound (I-6) thus obtained can be isolated and purified by a known means of separation and purification, such as concentration, concentration under reduced pressure, solvent extraction, crystallization, recrystallization, phase transfer, chromatography and the like.

The compound (I-5) to be used as a starting material in the above-mentioned METHOD D can be produced by, for example, the above-mentioned METHOD A-METHOD C. In addition, a known compound is used as compound (IV).

The compound (I-7), having a bond for Xb in the formula (I), can be produced by, for example, the following METHOD E. 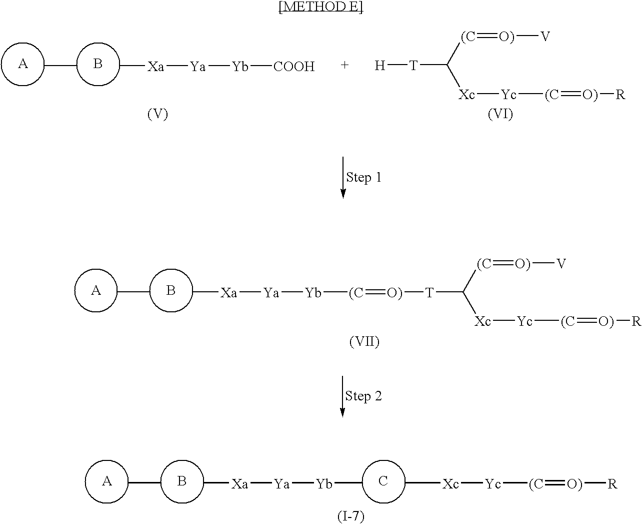
wherein T is —O—, —S— or —NR³— (R³ is as defined above), V is a hydrogen atom or a substituent, and other symbols are as defined above.

As the substituent represented by V, those exemplified as the substituent for the aforementioned ring C can be mentioned.

[Step 1]

This method is performed in the same manner as in the reaction between compound (I-5) and compound (IV) in the aforementioned METHOD D.

The compound (VII) thus obtained can be isolated and purified by a known means of separation and purification, such as concentration, concentration under reduced pressure, solvent extraction, crystallization, recrystallization, phase transfer, chromatography and the like. It is also possible to use a reaction mixture containing compound (VII) as a starting material for Step 2, without isolating compound (VII).

The compound (V) to be used as a starting material in Step 1 of the above-mentioned METHOD E can be produced by, for example, a method described in WO 01/38325 and the like, or a method analogous thereto. The compound (VI) can be produced by a known method.

[Step 2]

In this method, compound (VII) is subjected to ring closure reaction to give compound (I-7).

This reaction is carried out according to a conventional method in the presence of an ammonium salt in a solvent which does not interfere with the reaction.

As the ammonium salt, for example, ammonium acetate and the like can be mentioned.

The amount of the ammonium salt to be used is generally 0.1-10 molar equivalents, preferably 0.3-5 molar equivalents, relative to compound (VII).

As the solvent which does not interfere with the reaction, for example, ethers such as diethyl ether, tetrahydrofuran, dioxane and the like; halogenated hydrocarbons such as chloroform, dichloromethane and the like; aromatic hydrocarbons such as benzene, toluene, xylene and the like; amides such as N,N-dimethylformamide and the like; alcohols such as ethanol, methanol and the like; organic acids such as acetic acid and the like; and the like can be mentioned. These solvents may be used after mixing at a suitable ratio.

The reaction temperature is generally −50° C. to about 200° C., preferably about −10° C. to about 150° C.

The reaction time is generally about 0.5-about 20 hours.

The compound (I-7) thus obtained can be isolated and purified by a known means of separation and purification, such as concentration, concentration under reduced pressure, solvent extraction, crystallization, recrystallization, phase transfer, chromatography and the like.

Of compounds (II) used as a starting material in the above-mentioned METHOD A, compound (II-1), having —(CH₂)ₙ—CH₂— (n is an integer of 0 to 5) for Ya, and a hydroxy group for E, can be also produced by, for example, the following METHOD F. 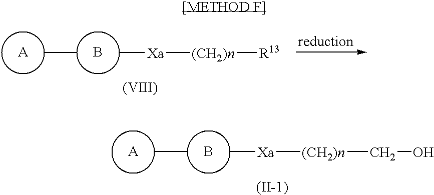
wherein R¹³ is CHO or COOR¹⁴ (R¹⁴ is an alkyl group having 1 to 6 carbon atoms), and other symbols are as defined above.

As the alkyl group group having 1 to 6 carbon atoms represented by R¹⁴, those exemplified for the aforementioned R¹¹ are used.

In this method, compound (VIII) is subjected to reduction to give compound (II-1).

This reaction is generally carried out in the presence of a reducing agent in a solvent that does not interfere with the reaction.

As the reducing agent, for example, metal hydride compounds such as sodium bis(2-methoxyethoxy)aluminum hydride, diisobutylaluminum hydride and the like; metal hydride complex compounds such as sodium borohydride, sodium cyanoborohydride, lithium aluminum hydride, sodium aluminum hydride and the like; and the like can be mentioned.

The amount of the reducing agent to be used is generally 1 to 20 molar equivalents relative to compound (VIII).

As the solvent that does not interfere with the reaction, for example, alcohols such as methanol, ethanol, propanol, 2-propanol, butanol, isobutanol, tert-butanol and the like; aromatic hydrocarbons such as benzene, toluene, xylene and the like; aliphatic hydrocarbons such as hexane, heptane and the like; ethers such as diethyl ether, diisopropyl ether, tert-butyl methyl ether, tetrahydrofuran, dioxane, dimethoxyethane and the like; amides such as N,N-dimethylformamide, N,N-dimethylacetamide, N-methylpyrrolidone and the like; halogenated hydrocarbons such as dichloromethane, chloroform, 1,2-dichloroethane, 1,1,2,2-tetrachloroethane and the like; and the like can be mentioned. These solvents may be used after mixing at an appropriate ratio.

The reaction temperature is generally −70° C. to 150° C., preferably −20° C. to 100° C.

The reaction time is generally 0.1-100 hrs, preferably 0.1-40 hrs.

The compound (II-1) thus obtained can be isolated and purified by a known separation and purification means, such as concentration, concentration under reduced pressure, solvent extraction, crystallization, recrystallization, phase transfer, chromatography and the like.

In the present invention, a compound represented by the formula 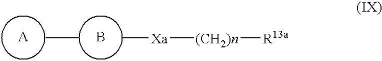
wherein R^{13a} is CH₂OH, CHO or COOR¹⁴ (R¹⁴ is as defined above), and other symbols are as defined above, and a salt thereof are useful as starting materials for the aforementioned METHOD A and METHOD F.

Of compounds (VIII) used as a starting material in the above-mentioned METHOD F, compound (VIII-1), having a bond for Xa, and na (na is an integer of 2 to 5) for n, can be also produced by, for example, the following METHOD G. 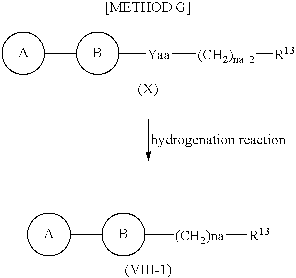
wherein Yaa is —CH═CH— or —C≡C—, and other symbols are as defined above.

In this method, compound (X) is subjected to hydrogenation reaction to give compound (VIII-1).

This reaction can be carried out in the presence of a metal catalysts such as palladium-carbon, palladium black, palladium chloride, platinum oxide, platinum black, platinum-palladium, Raney-nickel, Raney-cobalt and the like and a hydrogen source in a solvent that does not interfere with the reaction.

The amount of the metal catalyst to be used is generally 0.001 to 1000 molar equivalents, preferably 0.01 to 100 molar equivalents, relative to compound (X).

As the hydrogen source, for example, hydrogen gas, formic acid, formic acid amine salts, phosphinic acid salts, hydrazine and the like can be mentioned.

As the solvent that does not interfere with the reaction, those exemplified for the aforementioned METHOD F are used.

The reaction temperature and the reaction time are the same as those in the aforementioned METHOD F.

The compound (VIII-1) thus obtained can be isolated and purified by a known separation and purification means, such as concentration, concentration under reduced pressure, solvent extraction, crystallization, recrystallization, phase transfer, chromatography and the like.

Of compounds (VIII) used as a starting material in the above-mentioned METHOD F, compound (VIII-2), having a bond for Xa, and 0 for n, can be also produced by, for example, a method described in WO 01/38325 and the like, or a method analogous thereto.

Of compounds (X) used as a starting material in the above-mentioned METHOD G, compound (X-1), having COOR¹⁴ (R¹⁴ is as defined above) for R¹³, can be also produced by, for example, the following METHOD H. 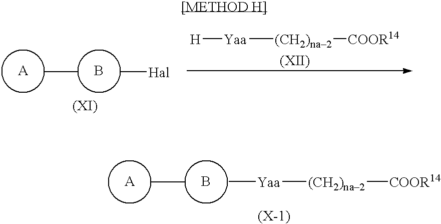
wherein Hal is a halogen atom, and other symbols are as defined above.

As the halogen atom represented by Hal, for example, fluorine, chlorine, bromine, iodine and the like can be mentioned. Of these, bromine, iodine and the like are preferable.

In this method, compound (XI) is reacted with compound (XII) to give compound (X-1).

This reaction is generally carried out in the presence of a metal catalyst and a ligand in a solvent that does not interfere with the reaction.

As used herein, as the metal catalyst, for example palladium [e.g., divalent palladium salts and complex thereof, such as palladium acetate, palladium chloride, palladium bromide, palladium iodide, bis(triphenylphosphine)palladium(II) chloride, bis(acetonyl)palladium(II) chloride, palladium trifluoroacetate and the like; non-valent palladium and complex thereof such as palladium carbon, palladium black, tetrakistriphenylphosphinepalladium, bis(benzalacetone)palladium(0) and the like], nickel (e.g., nickel acetate, nickel chloride), cobalt (e.g., cobalt chloride) and the like can be mentioned.

As the ligand, for example, phosphines (e.g., trimethylphosphine, triethylphosphine, tri-n-butylphosphine, tri-tert-butylphosphine, triphenylphosphine, tri-o-tolylphosphine, tri-p-tolylphosphine, BINAP [2,2′-bis(diphenylphosphino)-1,1′-binaphthyl], tri(2-furyl)phosphine, tri(2-thienyl)phosphine, 1,2-bis(diphenylphosphino)ethane, 1,2-bis(diphenylphosphino)propane, 1,2-bis(diphenylphosphino)butane and the like) and the like can be mentioned.

As the solvent that does not interfere with the reaction, for example, aromatic hydrocarbons such as benzene, toluene, xylene and the like; ethers such as dioxane, tetrahydrofuran, dimethoxyethane and the like; alcohols such as methanol, ethanol, propanol, isopropanol, butanol, tert-butanol and the like; esters such as methyl acetate, ethyl acetate, butyl acetate and the like; nitriles such as acetonitrile, propionitrile and the like; ketones such as acetone, 2-butanone, 2-pentanone and the like; amides such as N,N-dimethylformamide, N,N-dimethylacetamide, N-methylpyrrolidone, N,N-dimethylimidazolidinone and the like; halogenated hydrocarbons such as dichloromethane, chloroform, 1,2-dichloroethane, 1,1,2,2-tetrachloroethane and the like; sulfoxides such as dimethyl sulfoxide and the like; water and the like are used. These solvents may be used after mixing at an appropriate ratio.

For the purpose of promoting the reaction, this reaction may be carried out in the presence of a base or a quaternary ammonium salt. As the base, for example, alkali metal salts or alkaline earth metal salts (e.g., potassium hydroxide, sodium hydroxide, potassium carbonate, sodium carbonate, cesium carbonate, potassium hydrogen carbonate, sodium hydrogen carbonate, potassium acetate, sodium acetate, calcium acetate, potassium propionate, sodium propionate), metal hydrides (e.g., potassium hydride, sodium hydride, calcium hydride), amines (e.g., trimethylamine, triethylamine, diisopropylethylamine, tripropylamine, N-methylmorpholine, 1,8-diazabicyclo[5.4.0-7-undecene (DBU), 1,4-diazabicyclo[2,2,2]octane (DABCO), proton sponge, 4-dimethylaminopyridine, 4-diethylaminopyridine, pyridine, picoline, quinoline) and the like can be mentioned. As the quaternary ammonium salt, for example, tetraethylammonium chloride, tetraethylammonium bromide, benzyltrimethylammonium chloride, benzyltrimethylammonium bromide and the like can be mentioned.

The amount of compound (XII) to be used is generally 1 to 100 molar equivalents, preferably 1-10 molar equivalents, relative to compound (XI).

While the amount of the metal catalyst and ligand to be used varies depending on the reaction conditions, it is generally 0.00001-100 molar equivalents, preferably 0.0001-10 molar equivalents, relative to compound (XI).

The amount of the base or quaternary ammonium salt to be used is generally 0.01-100 molar equivalents, preferably 0.1-10 molar equivalents, relative to compound (XI).

The reaction temperature is generally −30° C. to 200° C., preferably −10° C. to 150° C.

The reaction time is generally 0.1-100 hrs, preferably 0.1-40 hrs.

The compound (X-1) thus obtained can be isolated and purified by a known separation and purification means, such as concentration, concentration under reduced pressure, solvent extraction, crystallization, recrystallization, phase transfer, chromatography and the like.

The above-mentioned compound (XII) can be produced according to a method known per se.

Of the aforementioned compounds (X-1), compound (X-1a), having —CH═CH— for Yaa, 2 for na, can be also produced by reacting, from among the compounds (VIII) used as a starting material in the above-mentioned METHOD F, compound (VIII-2a), having a bond for Xa, 0 for n, and CHO for R¹³, with an organic phosphorus reagent.

This reaction is generally carried out according to the conventional method in the presence of a base in a solvent that does not interfere with the reaction.

As the organic phosphorus reagent, for example, methyl dimethylphosphonoacetate, ethyl diethylphosphonoacetate, ethyl dimethylphosphonoacetate and the like can be mentioned.

The amount of the organic phosphorus reagent to be used is preferably about 1-about 10 molar equivalents relative to compound (VIII-2a).

As the solvent that does not interfere with the reaction, those exemplified for the reaction in the aforementioned METHOD A when E is a halogen atom or —OSO₂R¹¹ can be used. The amount of the base to be used, reaction temperature and reaction time are the same as those in said reaction.

The compound (X-1a) thus obtained can be isolated and purified by a known separation and purification means, such as concentration, concentration under reduced pressure, solvent extraction, crystallization, recrystallization, phase transfer, chromatography and the like.

The above-mentioned compound (VIII-2a) can be also produced by subjecting, from among the compounds (II-1) produced in the above-mentioned METHOD F, compound (II-1a), having a bond for Xa, and 0 for n, to oxidation reaction.

The oxidation reaction is generally carried out according to a conventional method in the presence of an oxidizing agent in a solvent that does not interfere with the reaction.

As the oxidizing agent, for example, metal oxidizing agents such as manganese dioxide, pyridinium chlorochromate, pyridinium dichromate, ruthenium oxide and the like, and the like can be mentioned.

The amount of the oxidizing agent to be used is preferably about 1-about 10 molar equivalents relative to compound (II-1a).

As the solvent that does not interfere with the reaction, for example, aromatic hydrocarbons such as benzene, toluene, xylene and the like; ethers such as tetrahydrofuran, dioxane, diethyl ether and the like; halogenated hydrocarbons such as chloroform, dichloromethane and the like; and the like can be mentioned. These solvents may be used after mixing at an appropriate ratio.

The reaction temperature is generally about −50° C. to about 150° C., preferably about −10° C. to about 100° C.

The reaction time is generally about 0.5-about 20 hrs.

In addition, compound (VIII-2a) can be also produced by adding a reaction reagent such as sulfur trioxide pyridine complex or oxalyl chloride and the like to compound (II-1a) in dimethyl sulfoxide or a mixed solvent of dimethyl sulfoxide and a halogenated hydrocarbon such as chloroform, dichloromethane and the like, and reacting the resulting compound with an organic base such as triethylamine, N-methylmorpholine and the like.

The amount of the reaction reagent to be used is preferably about 1-about 10 molar equivalents relative to compound (II-1a).

The amount of the organic base to be used is preferably about 1-about 10 molar equivalents relative to compound (II-1a).

The reaction temperature is generally about −50° C. to about 150° C., preferably about −10° C. to about 100° C.

The reaction time is generally about 0.5-about 20 hrs.

The compound (VIII-2a) thus obtained can be isolated and purified by a known separation and purification means, such as concentration, concentration under reduced pressure, solvent extraction, crystallization, recrystallization, phase transfer, chromatography and the like.

Of compound (VIII), compound (VIII-3), having a bond for Xa, 2 for n, and CHO for R¹³, can be produced by using allyl alcohol instead of compound (XII) in the aforementioned METHOD H.

The compound (VIII-3) thus obtained can be isolated and purified by a known separation and purification means, such as concentration, concentration under reduced pressure, solvent extraction, crystallization, recrystallization, phase transfer, chromatography and the like.

The compound (XI) used as a starting material in the above-mentioned METHOD H can be produced by, for example, the following METHOD I. 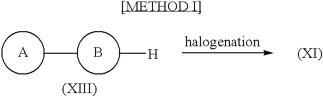
wherein the symbols in the formula are as defined above.

In this method, compound (XIII) is subjected to halogenation to give compound (XI).

This reaction is carried out according to a method known per se, for example, a method described in Tetrahedron Letters, vol. 42, page 863 (2001); Journal of Heterocyclic Chemistry, vol. 32, page 1351 (1995) and the like, or a method analogous thereto.

This reaction can be also carried out using a halogenating agent in a solvent that does not interfere with the reaction.

As the halogenating agent, for example, bromine, iodine, N-bromosuccinimide, N-iodosuccinimide, N-chlorosuccinimide, sulfuryl chloride and the like can be mentioned.

The amount of the halogenating agent to be used is generally 1 to about 20 molar equivalents relative to compound (XIII).

As the solvent that does not interfere with the reaction, for example, aromatic hydrocarbons such as benzene, toluene, xylene and the like; ethers such as tetrahydrofuran, dioxane, diethyl ether and the like; halogenated hydrocarbons such as chloroform, dichloromethane and the like; nitrites such as acetonitrile, propionitrile and the like; amides such as N,N-dimethylformamide, N,N-dimethylacetamide, N-methylpyrrolidone, N,N-dimethylimidazolidinone and the like; carboxylic acids such as acetic acid, propionic acid and the like; and the like can be mentioned. These solvents may be used after mixing at an appropriate ratio. The reaction temperature is generally about −20° C. to 150° C., preferably about 0° C. to about 100° C.

The reaction time is generally about 0.1-about 20 hrs.

The compound (XI) thus obtained can be isolated and purified by a known separation and purification means, such as concentration, concentration under reduced pressure, solvent extraction, crystallization, recrystallization, phase transfer, chromatography and the like.

The compound (XIII) used as a starting material in the above-mentioned METHOD I can be produced according to a method known per se, for example, a method described in Heterocycles, vol. 22, page 859 (1984); Journal of Organic Chemistry, vol. 48, page 3807 (1983); Tetrahedron Letters, vol. 34, page 75 (1993) and the like, or a method analogous thereto.

Of compounds (XIII), compound (XIII-1), having a pyrazole ring for 1,2-azole ring represented by ring B, can be also produced by, for example, the following METHOD J. 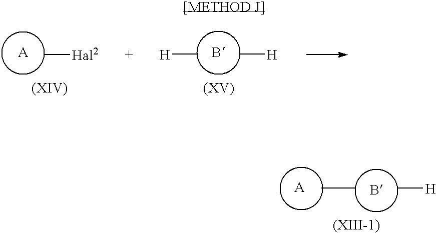
wherein Hal² is a halogen atom, B′ is a pyrazole ring optionally further having 1 to 3 substituents, and other symbols are as defined above.

As used herein, as the halogen atom represented by Hal², for example, fluorine, chlorine, bromine, iodine and the like can be mentioned. Of these, fluorine, chlorine, bromine and the like are preferable.

As the “pyrazole ring optionally further having 1 to 3 substituents” represented by B′, the “1,2-azole ring optionally further having 1 to 3 substituents” exemplified by the aforementioned B, wherein the 1,2-azole ring is a pyrazole ring can be mentioned.

In this method, compound (XIV) is reacted with compound (XV) to give compound (XIII-1).

This reaction is carried out in the same manner as in the reaction in the aforementioned METHOD A when E is a halogen atom or —OSO₂R¹¹.

The compound (XIII-1) thus obtained can be isolated and purified by a known separation and purification means, such as concentration, concentration under reduced pressure, solvent extraction, crystallization, recrystallization, phase transfer, chromatography and the like.

The compound (XIV) and compound (XV) used as starting materials in the above-mentioned METHOD J can be produced according to a method known per se. For example, compound (XV) can be produced according to a method described in Inorganic Chemistry, vol. 28, page 1091 (1998); WO 02/44173 and the like, or a method analogous thereto.

Of the aforementioned compounds (I-4), compound (I-4a), having a bond for Xc, and —CH₂— for Yc, can be also produced by, for example, the following METHOD K. 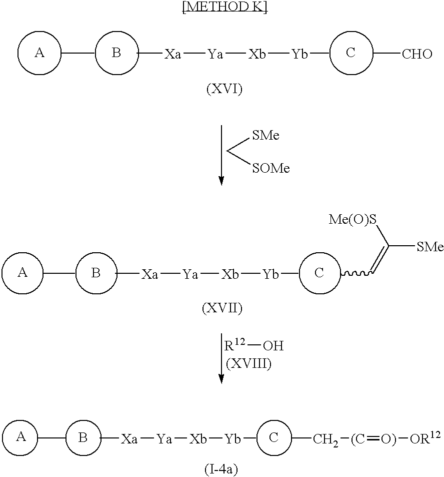
wherein the symbols in the formula are as defined above.

The optionally substituted hydrocarbon group represented by R¹² is preferably an alkyl group having 1 to 6 carbon atoms (e.g., methyl, ethyl, propyl, isopropyl, butyl, isobutyl, sec.-butyl, t.-butyl), aralkyl group having 7 to 13 carbon atoms (e.g., benzyl) and the like, more preferably methyl, ethyl and the like.

In this method, compound (XVI) is reacted with methyl methylthiomethyl sulfoxide (hereinafter to be abbreviated as FAMSO) to give compound (XVII), and said compound (XVII) is reacted with compound (XVIII) to give compound (I-4a).

This method can be performed according to a method known per se, for example, a method described in Journal of Organic Chemistry, vol. 47, page 5404 (1982) and the like, or a method analogous thereto.

For example, the reaction of compound (XVI) with FAMSO is generally carried out in the presence of a base in a solvent that does not interfere with the reaction. This reaction is carried out in the same manner as in the reaction in the aforementioned METHOD A when E is a halogen atom or —OSO₂R¹¹.

The compound (XVII) thus obtained can be isolated and purified by a known separation and purification means, such as concentration, concentration under reduced pressure, solvent extraction, crystallization, recrystallization, phase transfer, chromatography and the like.

The reaction of compound (XVII) and compound (XVIII) is generally carried out in the presence of an acid.

As used herein, as the acid, mineral acids such as hydrochloric acid, hydrobromic acid, sulfuric acid and the like; acidic gas such as hydrogen chloride gas, hydrogen bromide gas and the like; organic acids such as acetic acid, propionic acid and the like; and the like are used. The amount of the acid to be used is generally 0.01-100 molar equivalents, preferably 0.1-10 molar equivalents, relative to compound (XVII).

The reaction temperature is −30° C. to 200° C., preferably −10° C. to 150° C.

The reaction time is generally about 0.1-about 20 hrs.

This reaction may be carried out in a solvent used in the reaction of the aforementioned compound (XVI) with FAMSO.

The compound (I-4a) thus obtained can be isolated and purified by a known separation and purification means, such as concentration, concentration under reduced pressure, solvent extraction, crystallization, recrystallization, phase transfer, chromatography and the like.

The compound (XVI) used as a starting material in the aforementioned METHOD K can be produced by, for example, the following METHOD L. 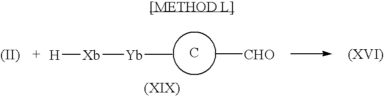
wherein the symbols in the formula are as defined above.

In this method, compound (II) is reacted with compound (XIX) to give compound (XVI). This reaction is carried out in the same manner as in the aforementioned METHOD A.

The compound (XVI) thus obtained can be isolated and purified by a known separation and purification means, such as concentration, concentration under reduced pressure, solvent extraction, crystallization, recrystallization, phase transfer, chromatography and the like.

The above-mentioned compound (XIX) can be produced according to a method known per se.

In each of the aforementioned reactions, when the starting material has an amino group, a carboxyl group, a hydroxyl group or a carbonyl group as a substituent, a protective group generally used in the peptide chemistry and the like may be introduced into these groups. After reaction, the protective group can be removed as necessary to give the object compound.

As the amino-protecting group, those exemplified as the aforementioned R³ can be mentioned.

As the carboxyl-protecting group, for example, C₁₋₆ alkyl group (e.g., methyl, ethyl, propyl, isopropyl, butyl, tert-butyl), C₇₋₁₁ aralkyl group (e.g., benzyl), phenyl group, trityl group, silyl group (e.g., trimethylsilyl, triethylsilyl, dimethylphenylsilyl, tert-butyldimethylsilyl, tert-butyldiethylsilyl), C₂₋₆ alkenyl group (e.g., 1-allyl) and the like can be mentioned. These groups may be substituted by 1 to 3 substituents selected from halogen atom (e.g., fluorine, chlorine, bromine, iodine), C₁₋₆ alkoxy group (e.g., methoxy, ethoxy, propoxy), nitro group and the like.

As the hydroxy-protecting group, those exemplified as the aforementioned R² can be mentioned.

Examples of the protective groups for carbonyl include cyclic acetals (e.g., 1,3-dioxane) and non-cyclic acetals (e.g., di-C₁₋₆ alkyl acetals).

In addition, these protective groups can be removed by a method known per se, e.g., the method described in Protective Groups in Organic Synthesis, published by John Wiley and Sons (1980). For example, there may be used methods employing an acid, a base, ultraviolet rays, hydrazine, phenylhydrazine, sodium N-methyldithiocarbamate, tetrabutylammonium fluoride, palladium acetate, a trialkylsilyl halide (e.g., trimethylsilyl iodide, trimethylsilyl bromide), or the like, the reduction method, and the like.

When compound (I) contains an optical isomer, a stereomer, a position isomer, or a rotation isomer, these isomers are also contained as Compound (I) and can each be obtained as a single substance by means of a method known per se of synthesis or separation. For example, when an optical isomer is present in Compound (I), the optical isomer separated from said compound is also included in Compound (I).

Optical isomers can be produced by a method known per se. Specifically, optical isomers are obtained by using an optically active synthesis intermediate, or optically resolving a racemate of the final product by a conventional method.

Examples of the methods of optical resolution include methods known per se, such as the fractional recrystallization method, the chiral column method, and the diastereomer method.

1) Fractional Recrystallization Method

A method wherein a salt is formed between a racemate and an optically active compound [e.g., (+)-mandelic acid, (−)-mandelic acid, (+)-tartaric acid, (−)-tartaric acid, (+)-1-phenethylamine, (−)-1-phenethylamine, cinchonine, (−)-cinchonidine, brucine], which salt is separated by fractional recrystallization, etc., and, if desired, subjected to a neutralization process, to yield a free optical isomer.

2) Chiral Column Method

A method wherein a racemate or a salt thereof is applied to a column for optical isomer separation (chiral column). In the case of liquid chromatography, for example, optical isomers are separated by adding a mixture of the optical isomers to a chiral column such as ENANTIO-OVM (produced by Tosoh Corporation) or CHIRAL series produced by DAICEL CHEMICAL IND., and developing it in water, various buffers (e.g., phosphate buffer), an organic solvent (e.g., ethanol, methanol, isopropanol, acetonitrile, trifluoroacetic acid, diethylamine), or a solvent mixture thereof. In the case of gas chromatography, for example, a chiral column such as CP-Chirasil-DeX CB (produced by GL Science) is used to separate optical isomers.

3) Diastereomer Method

A method wherein a racemate mixture and an optically active reagent are chemically reacted to yield a diastereomer mixture, which is then subjected to ordinary means of separation (e.g., fractional recrystallization, chromatography) to obtain single substances, which are subjected to a chemical reaction such as hydrolysis reaction to cut off the optically active reagent moiety, whereby the desired optical isomer is obtained. For example, when Compound (I) has hydroxy or primary or secondary amino in the molecule thereof, said compound, an optically active organic acid (e.g., MTPA [α-methoxy-α-(trifluoromethyl)phenylacetic acid], (−)-menthoxyacetic acid) and the like may be subjected to a condensation reaction to yield a diastereomer of an ester or amide, respectively. On the other hand, when Compound (I) has a carboxyl group, said compound and an optically active amine or an alcohol reagent may be subjected to a condensation reaction to yield a diastereomer of an amide or ester, respectively. The diastereomer thus separated is converted to an optical isomer of the original compound by subjecting it to an acid hydrolysis or basic hydrolysis reaction.

### EXAMPLES

The present invention is hereinafter described in more detail by means of, but is not limited to, the following Test Examples, Reference Examples, Examples and Preparation Examples.

In addition, % in the Reference Examples and Examples below means percent by weight, unless mentioned otherwise. Room temperature means the temperature of 1 to 30° C.

Abbreviations for bases, amino acids and others used in the present specification are based on abbreviations specified by the IUPAC-IUB Commission on Biochemical Nomenclature or abbreviations in common use in relevant fields. Some examples are given below. When an optical isomer may be present in amino acid, it is of the L-configuration, unless otherwise mentioned.

The sequence numbers in the sequence listing in the present specification show the following respective sequences.

[SEQ ID NO:1]

Shows the base sequence of the primer PARD-U used in Reference Example 1a.

[SEQ ID NO:2]

Shows the base sequence of the primer PARD-L used in Reference Example 1a.

[SEQ ID NO:3]

Shows the base sequence of the primer XRA-U used in Reference Example 2a.

[SEQ ID NO:4]

Shows the base sequence of the primer XRA-L used in Reference Example 2a.

[SEQ ID NO:5]

Shows the base sequence of the primer PPRE-U used in Reference Example 5a.

[SEQ ID NO:6]

Shows the base sequence of the primer PPRE-L used in Reference Example 5a.

[SEQ ID NO:7]

Shows the base sequence of the primer TK-U used in Reference Example 5a.

[SEQ ID NO:8]

Shows the base sequence of the primer TK-L used in Reference Example 5a.

[SEQ ID NO:9]

Shows the base sequence of the primer PAG-U used in Reference Example 6a.

[SEQ ID NO:10]

Shows the base sequence of the primer PAG-L used in Reference Example 6a.

[SEQ ID NO:11]

Shows the base sequence of the sense chain primer used in Reference Example 10a.

[SEQ ID NO:12]

Shows the base sequence of the antisense chain primer used in Reference Example 10a.

### Test Example 1

Hypoglycemic and Hypolipidemic Actions in Mice

Test compounds were mixed in a powdery diet (CE-2, Japan Clea) at the concentration of 0.005%, and freely given to KKAY mice (9 to 12 weeks old, 5 mice in a group), a model of obese and non-insulin dependent diabetes (type 2 diabetes), for four days. During this period, water was given freely. Blood was sampled from orbital venous plexus, and glucose and triglyceride levels in plasma separated from blood were determined enzymatically using L type Wako Glu2 (Wako Pure Chemical Industries, Ltd.) or L type Wako TG·H (Wako Pure Chemical Industries, Ltd.), respectively. The results are given in Table 1.

In the table, “hypoglycemic action (%)” means the rate of decrease (%) in the blood glucose level of the treated group when the blood glucose level of the non-treated group is taken as 100%. In addition, the “hypolipidemic action (%)” means the rate of decrease (%) in the blood triglyceride level of the treated group when the blood triglyceride level of the non-treated group is taken as 100%.

TABLE 1

| Test compound | Hypoglycemic action | Hypolipidemic action |
|---|---|---|
| (Example No.) | (%) | (%) |
| 28 | 42 | 56 |
| 29 | 46 | 65 |
| 30 | 35 | 58 |
| 31 | 50 | 69 |
| 34 | 49 | 77 |
| 35 | 30 | 32 |
| 41 | 25 | 48 |
| 42 | 32 | 19 |
| 179 | 34 | 37 |
| 180 | 32 | 36 |
| 181 | 49 | 49 |
| 185 | 47 | 43 |
| 189 | 50 | 38 |
| 197 | 45 | 65 |
| 207 | 49 | 72 |
| 212 | 52 | 55 |
| 213 | 51 | 52 |
| 214 | 44 | 52 |
| 215 | 50 | 45 |
| 216 | 46 | 61 |
| 217 | 34 | 18 |
| 218 | 34 | 43 |
| 220 | 44 | 50 |
| 221 | 46 | 21 |
| 222 | 36 | 54 |
| 223 | 38 | 55 |
| 224 | 48 | 60 |
| 225 | 31 | 35 |
| 227 | 43 | 26 |
| 229 | 41 | 49 |
| 235 | 43 | 64 |
| 239 | 42 | 65 |
| 241 | 38 | 27 |
| 245 | 42 | 55 |
| 247 | 25 | 34 |
| 253 | 49 | 35 |
| 259 | 34 | 70 |
| 260 | 42 | 44 |
| 272 | 48 | 69 |
| 274 | 50 | 60 |
| 300 | 36 | 39 |
| 305 | 50 | 55 |
| 311 | 52 | 29 |
| 313 | 51 | 48 |
| 315 | 53 | 70 |
| 337 | 44 | 48 |
| 339 | 50 | 54 |
| 340 | 49 | 55 |
| 351 | 51 | 49 |

These results indicated that the compounds of the present invention possess excellent hypoglycemic and hypolipidemic actions, and are proved to be useful as agents for preventing or treating diabetes, hyperlipidemia (especially hypertriglyceridemia), impaired glucose tolerance, etc.

### Test Example 2

Plasma Anti-Arteriosclerosis Index-Enhancing Action in Mice

Test compounds were mixed in a powdery diet (CE-2, Japan Clea) at the concentration of 0.005%, and freely given to KKAY mice (9 to 12 weeks old, 5 mice per group), a model of obese and non-insulin dependent diabetes (type 2 diabetes), for four days. During this period, water was given freely. Blood was sampled from orbital venous plexus and components in plasma separated from blood were determined. Total cholesterol levels were determined by using L type Wako Cholesterol (Wako Pure Chemical Industries, Ltd.). Precipitation reagent for HDL cholesterol (Wako Pure Chemical Industries, Ltd.) was added to a part of the plasma to precipitate non-HDL lipoprotein, and cholesterol (HDL cholesterol) in the resulting supernatant was determined. The plasma anti-arteriosclerosis index [(HDL cholesterol/total cholesterol)×100] was calculated by using these cholesterol levels. The results are given in Table 2.

In the Table, “Plasma anti-arteriosclerosis index-enhancing action (%)” represents the percent increase (%) of plasma anti-arteriosclerosis index in the treatment group, when the plasma anti-arteriosclerosis index in the non-treatment group is taken as 100%.

TABLE 2

| | Plasma anti- |
|---|---|
| Test compound | arteriosclerosis index- |
| (Example No.) | enhancing action (%) |
| 22 | 12 |
| 28 | 18 |
| 29 | 23 |
| 30 | 19 |
| 31 | 16 |
| 34 | 20 |
| 35 | 14 |
| 41 | 12 |
| 185 | 15 |
| 189 | 20 |
| 223 | 12 |
| 224 | 14 |
| 225 | 12 |
| 253 | 16 |
| 259 | 25 |
| 260 | 22 |
| 274 | 11 |
| 299 | 11 |
| 300 | 12 |
| 302 | 24 |
| 303 | 14 |
| 304 | 13 |
| 305 | 22 |
| 313 | 15 |
| 315 | 11 |
| 316 | 10 |
| 318 | 22 |
| 322 | 14 |
| 332 | 11 |
| 333 | 11 |
| 335 | 12 |
| 337 | 24 |
| 339 | 22 |
| 340 | 21 |

These results indicated that the compounds of the present invention possess excellent total cholesterol lowering actions, and are proved to be useful as agents for preventing or treating hyperlipidemia (especially hypercholesterolemia). Additionally, the compounds of the present invention possess excellent plasma anti-arteriosclerosis index-enhancing actions, and are proved to be useful as an agent for the prophylaxis or treatment of hyperlipidemia (especially hypo-HDL-cholesterolemia), arteriosclerosis, etc.

### Test Example 3

(PPARγ-RXRα Heterodimer Ligand Activity)

A PPARγ: RXRα: 4ERPP/CHO-K1 cells obtained in Reference Example 8a described later were cultured in HAM F12 medium (produced by Life Technologies, Inc., USA) containing 10% Fetal bovine serum (produced by Life Technologies, Inc., USA) and then inoculated to a 96-well white plate (produced by Corning Costar Corporation, USA) at the density of 2×10⁴ cells/well, and cultured in a CO₂ gas incubator at 37° C. overnight.

After removing the medium from 96 well white plate, 80 μl of HAM F12 medium containing 0.1% fatty acid-free bovine serum albumin (BSA) and 20 μl of test compound were added, which was cultured in a CO₂ gas incubator at 37° C. for 18-48 hours. After removing the medium, 40 μl of PIKKAGENE 7.5 (produced by Wako Pure Chemical Industries, Ltd.) diluted twice with HBSS (HANKS' BALANCED SALT SOLUTION)(produced by BIO WHITTAKER Inc., USA), was added. After stirring, the luciferase activity was determined using 1420 ARVO Multilabel Counter (produced by PerkinElmer Inc., USA).

A fold induction was calculated based on the luciferase activity of each test compound by taking the luciferase activity in the non-treatment group as 1. The values of the test compound concentration and the fold induction were analyzed using PRISM (produced by GraphPad Software Inc. USA) to calculate the EC₅₀ values, the effective concentration of a test compound for 50% of the maximum fold induction. The results are shown in Table 3.

TABLE 3

| Test compound | |
|---|---|
| (Example No.) | EC₅₀ (nM) |
| 24 | 38 |
| 28 | 35 |
| 29 | 160 |
| 30 | 210 |
| 31 | 35 |
| 41 | 77 |
| 42 | 19 |
| 43 | 53 |
| 58 | 43 |
| 77 | 21 |
| 98 | 110 |
| 104 | 34 |
| 116 | 82 |
| 125 | 26 |
| 137 | 35 |
| 181 | 75 |
| 189 | 14 |
| 196 | 42 |
| 197 | 22 |
| 198 | 30 |
| 201 | 63 |
| 210 | 16 |
| 212 | 13 |
| 213 | 7.8 |
| 214 | 18 |
| 215 | 20 |
| 216 | 18 |
| 218 | 51 |
| 220 | 9.6 |
| 221 | 12 |
| 223 | 24 |
| 227 | 22 |
| 229 | 21 |
| 235 | 26 |
| 237 | 17 |
| 239 | 35 |
| 245 | 19 |
| 259 | 76 |
| 270 | 99 |
| 271 | 30 |
| 272 | 50 |
| 273 | 90 |
| 274 | 82 |
| 277 | 36 |
| 302 | 37 |
| 303 | 52 |
| 304 | 40 |
| 306 | 17 |
| 307 | 23 |
| 311 | 100 |
| 315 | 35 |
| 316 | 3.8 |
| 319 | 26 |
| 332 | 29 |
| 333 | 61 |
| 334 | 74 |
| 340 | 22 |
| 351 | 20 |
| 367 | 41 |

These results indicated that the compounds of the present invention have potent PPARγ-RXRα heterodimer ligand activity.

### Test example 4

(PPARδ-RXRα Heterodimer Ligand Activity)

The transformant obtained in Reference Example 9a was suspended in DMEM medium (produced by Life Technologies, Inc., USA) containing 0.1% fatty acid-free bovine serum albumin (BSA) (produced by Wako Pure Chemical Industries, Ltd.), and inoculate to each well of a 96-well white plate (produced by Corning Costar Corporation, USA) by 80 μl at 1×10⁴ cells/well. Then the test compound (20 μl) was added and cultured at 37° C. under 5% CO₂ for 36-48 hours. After removing the medium from the 96-well white plate, 40 μl of PIKKAGENE LT 7.5 (produced by Wako Pure Chemical Industries, Ltd.) diluted twice with HBSS (HANKS' BALANCED SALT SOLUTION)(produced by BIO WHITTAKER Inc., USA), was added. After stirring, the luciferase activity was determined using 1420 ARVO Multilabel Counter (produced by PerkinElmer Inc., USA).

A fold induction was calculated based on the luciferase activity of each test compound by taking the luciferase activity in the non-treatment group as 1. The values of the test compound concentration and the fold induction were analyzed using PRISM (produced by GraphPad Software Inc. USA) to calculate the EC₅₀ values, the effective concentration of a test compound for 50% of the maximum fold induction. The results are shown in Table 4.

TABLE 4

| Test compound | |
|---|---|
| (Example No.) | EC₅₀ (nM) |
| 22 | 8.6 |
| 24 | 9.3 |
| 30 | 2.6 |
| 31 | 9.6 |
| 34 | 8.1 |
| 35 | 1.6 |
| 42 | 1.9 |
| 43 | 3.7 |
| 44 | 3.9 |
| 46 | 6.4 |
| 49 | 1.7 |
| 51 | 3.9 |
| 56 | 2.8 |
| 58 | 1.9 |
| 59 | 9.7 |
| 62 | 0.81 |
| 63 | 9.5 |
| 65 | 1.8 |
| 75 | 3.8 |
| 76 | 1.9 |
| 85 | 6.0 |
| 86 | 1.5 |
| 91 | 6.0 |
| 92 | 1.9 |
| 94 | 4.0 |
| 96 | 1.7 |
| 98 | 1.2 |
| 99 | 0.55 |
| 102 | 9.1 |
| 104 | 7.0 |
| 105 | 7.2 |
| 110 | 4.6 |
| 111 | 6.1 |
| 113 | 4.8 |
| 116 | 0.6 |
| 117 | 1.6 |
| 118 | 7.2 |
| 122 | 4.9 |
| 123 | 2.9 |
| 124 | 2.4 |
| 125 | 1.5 |
| 126 | 2.2 |
| 127 | 3.9 |
| 129 | 4.9 |
| 131 | 2.7 |
| 137 | 9.6 |
| 146 | 5.8 |
| 150 | 2.7 |
| 152 | 9.9 |
| 153 | 1.9 |
| 154 | 1.5 |
| 155 | 3.8 |
| 157 | 4.7 |
| 168 | 1.6 |
| 169 | 5.7 |
| 181 | 84 |
| 182 | 5.6 |
| 186 | 1.9 |
| 189 | 2.1 |
| 200 | 5.9 |
| 201 | 1.2 |
| 204 | 4.6 |
| 212 | 42 |
| 213 | 8.3 |
| 223 | 97 |
| 227 | 54 |
| 237 | 6.1 |
| 245 | 130 |
| 255 | 9.5 |
| 258 | 5.5 |
| 274 | 320 |
| 278 | 6.0 |
| 279 | 5.1 |
| 304 | 5.7 |
| 311 | 280 |
| 316 | 9.9 |
| 319 | 5.1 |
| 340 | 45 |
| 351 | 72 |
| 367 | 150 |

These results indicated that the compounds of the present invention have potent PPARδ-RXRα heterodimer ligand activity.

### Reference Example 1a

(Human PPARδ Gene Cloning)

A human PPARδ gene was cloned using a pancreas cDNA (produced by Toyobo Co., Ltd., trade name: QUICK-Clone cDNA) as a template by means of a PCR method employing a primer set shown below which was prepared with reference to the base sequence of PPARδ gene reported by Schmidt, A. et al (Mol. Endocrinol., 1992, Vol. 6, page 1634-1641).

PARD-U;5′-AAC GGT ACC TCA GCC ATG GAG CAG CCT CAG GAG G-3′ (SEQ ID NO:1)

PARD-L;5′-TAA GTC GAC CCG TTA GTA CAT GTC CTT GTA GAT C-3′ (SEQ ID NO: 2)

The PCR reaction was performed by Hot Start method using AMPLIWAX PCR Gem 100 (produced by TAKARA SHUZO CO., LTD.). First, 2 μl of 10×LA Buffer, 3 μl of 2.5 mM dNTP solution, 2.5 μl each of 12.5 μM primer solutions and 10 μl of sterilized distilled water were mixed to obtain a bottom layer solution mixture. 1 μl of human heart cDNA (1 ng/ml) as a template, 3 μl of 10×LA PCR Buffer, 1 μl of 2.5 mM dNTP solution, 0.5 μl of TaKaRa LA Taq DNA polymerase (produced by TAKARA SHUZO CO., LTD.) and 24.5 μl of sterilized distilled water were mixed to obtain a top layer solution mixture.

To the prepared bottom layer solution mixture, added was one unit of AmpliWax PCR Gem 100 (produced by TAKARA SHUZO CO., LTD.), which was treated at 70° C. for 5 minutes and then in ice for 5 minutes. Then, the top layer solution mixture was added to the mixture to prepare the reaction mixture of PCR. A tube containing the reaction mixture was set on a thermal cycler (produced by Perkin Elmer, USA) and treated at 95° C. for 2 minutes. After repeating the cycle of 95° C. for 15 seconds and 68° C. for 2 minutes a further 45 times, the tube was treated at 72° C. for 8 minutes.

The PCR product thus obtained was subjected to electrophoresis on agarose gel (1%), and 1.4 kb DNA fragment containing PPARδ gene was recovered from the gel, and then inserted into pT7 Blue-T vector (produced by TAKARA SHUZO CO., LTD.) to obtain a plasmid pTBT-hPPARδ.

### Reference Example 2a

(Human RXRα Gene Cloning)

A human RXRα gene was cloned using a kidney cDNA (produced by Toyobo Co., Ltd., trade name: QUICK-Clone cDNA) as a template by means of a PCR method employing a primer set shown below which was prepared with reference to the base sequence of RXRα gene reported by Mangelsdorf, D. J. et al (Nature, 1990, Vol. 345 (6272), page 224-229).

XRA-U:

(SEQ ID NO:3)

5′-TTA GAA TTC GAC ATG GAC ACC AAA CAT TTC CTG-3′
XRA-L:

(SEQ ID NO:4)

5′-CCC CTC GAG CTA AGT CAT TTG GTG CGG CGC CTC-3′

The PCR reaction was performed by Hot Start method using AmpliWax PCR Gem 100 (produced by TAKARA SHUZO CO., LTD.). First, 2 μl of 10×LA PCR Buffer, 3 μl of 2.5 mM dNTP solution, 2.5 μl each of 12.5 μM primer solutions and 10 μl of sterilized distilled water were mixed to obtain a bottom layer solution mixture. 1 μl of human kidney cDNA (1 ng/ml) as a template, 3 μl of 10×LA PCR Buffer, 1 μl of 2.5 mM DNTP solution, 0.5 μl of TaKaRa LA Taq DNA polymerase (produced by TAKARA SHUZO CO., LTD.) and 24.5 μl of sterilized distilled water were mixed to obtain a top layer solution mixture.

To the bottom layer solution mixture described above, added was one unit of AmpliWax PCR Gem 100 (produced by TAKARA SHUZO CO., LTD.), which was treated at 70° C. for 5 minutes and then in ice for 5 minutes. Then, the top layer solution mixture was added to the mixture to prepare the reaction mixture of PCR. A tube containing the reaction mixture was set on a thermal cycler (produced by Perkin Elmer, USA) and treated at 95° C. for 2 minutes. After repeating the cycle of 95° C. for 15 seconds and 68° C. for 2 minutes a further 35 times, the tube was treated at 72° C. for 8 minutes.

The PCR product thus obtained was subjected to electrophoresis on agarose gel (1%), and 1.4 kb DNA fragment containing RXRα gene was recovered from the gel, and then inserted into pT7 Blue-T vector (produced by TAKARA SHUZO CO., LTD.) to obtain a plasmid pTBT-hRXRα.

### Reference Example 3a

### Construction of Plasmids for Expressing Human PPARδ

pCI vector (produced by Promega, USA) was digested with BamHI (produced by TAKARA SHUZO CO., LTD.) and then treated with T4 DNA polymerase (produced by TAKARA SHUZO CO., LTD.) to obtain a blunt terminal. On the other hand, pGFP-C1 (produced by Toyobo Co., Ltd.) was digested with Bsu36I (produced by Daiichi Pure Chemicals CO., LTD.) and then treated with T4 DNA polymerase (produced by TAKARA SHUZO CO., LTD.) to form a blunt terminal, the both DNA fragments were ligated using DNA Ligation kit (produced by TAKARA SHUZO CO., LTD.) to obtain the plasmid pMCMVneo. A 5.6 Kb KpnI-SalI fragment of plasmid pMCMVneo was ligated to a 1.3 kb KpnI-SalI fragment containing hPPARδ gene of plasmid pTBT-hPPARδ described in Reference Example 1a to construct a plasmid pMCMVneo-hPPARδ.

### Reference Example 4a

### Construction of Plasmids for Expressing Human RXRa

A 5.6 Kb EcoRI-SalI fragment of plasmid pMCMVneo described in Reference Example 3a was ligated to a 1.4 kb EcoRI-XhoI fragment containing hRXRa gene of plasmid pTBT-hRXRa described in Reference Example 2a to prepare plasmid pMCMVneo-hRXRa.

### Reference Example 5a

(Construction of Reporter Plasmids)

A DNA fragment containing PPAR-responding element (PPRE) of an acyl CoA oxidase was prepared using the following 5′-terminal phosphorylated synthetic DNA.

PPRE-U:

(SEQ ID NO:5)

5′-pTCGACAGGGGACCAGGACAAAGGTCACGTTCGGGAG-3′
PPRE-L:

(SEQ ID NO:6)

5′-pTCGACTCCCGAACGTGACCTTTGTCCTGGTCCCCTG-3′

First, PPRE-U and PPRE-L were annealed and inserted to Sal I site of plasmid pBlue Script SK+. By determining the base sequence of the inserted fragment, plasmid pBSS-PPRE4 in which 4 PPREs were ligated in tandem was selected.

A HSV thymidine kinase minimum promoter (TK promoter) region was cloned using pRL-TK vector (produced by Promega, USA) as a template by means of a PCR method employing a primer set shown below which was prepared with reference to the base sequence of the promoter region of thymidine kinase reported by Luckow, B et al (Nucleic Acids Res., 1987, Vol. 15 (13), p. 5490)

TK-U:

(SEQ ID NO:7)

5′-CCCAGATCTCCCCAGCGTCTTGTCATTG-3′
TK-L:

(SEQ ID NO:8)

5′-TCACCATGGTCAAGCTTTTAAGCGGGTC-3′

The PCR reaction was performed by Hot Start method using AmpliWax PCR Gem 100 (TAKARA SHUZO CO., LTD.). First, 2 μl of 10×LA PCR Buffer, 3 μl of 2.5 mM dNTP solution, 2.5 μl each of 12.5 μM primer solutions and 10 μl of sterilized distilled water were mixed to obtain a bottom layer solution mixture. 1 μl of pRL-TK vector (produced by Promega, USA) as a template, 3 μl of 10×LA PCR Buffer, 1 μl of 2.5 mM DNTP solution, 0.5 μl of TaKaRa LA Taq DNA polymerase (produced by TAKARA SHUZO CO., LTD.) and 24.5 μl of sterilized distilled water were mixed to obtain a top layer solution mixture.

To the bottom layer solution mixture described above, added was one unit of AmpliWax PCR Gem 100 (produced by TAKARA SHUZO CO., LTD.), which was treated at 70° C. for 5 minutes and then in ice for 5 minutes. Then, the top layer solution mixture was added to the mixture to prepare the reaction mixture of PCR. A tube containing the reaction mixture was set on a thermal cycler (produced by Perkin Elmer, USA) and treated at 95° C. for 2 minutes. After repeating the cycle of 95° C. for 15 seconds and 68° C. for 2 minutes a further 35 times, the tube was treated at 72° C. for 8 minutes.

The PCR product thus obtained was subjected to electrophoresis on agarose gel (1%), and 140 b DNA fragment containing TK promoter was recovered from the gel, and then inserted into pT7 Blue-T vector (produced by TAKARA SHUZO CO., LTD.). By digesting the plasmid thus obtained with the restriction enzymes Bg1 II and NcoI, a fragment containing TK promoter was obtained, which was ligated to the Bg1 II-NcoI fragment of plasmid pGL3-Basic vector (produced by Promega, USA) to obtain plasmid pGL3-TK.

A 4.9 kb NheI-XhoI fragment of plasmid pGL3-TK thus obtained was ligated to a 200 bp NheI-XhoI fragment of plasmid pBSS-PPRE4 to obtain plasmid pGL3-4ERPP-TK.

This plasmid pGL3-4ERPP-TK was digested with BamHI (produced by TAKARA SHUZO CO., LTD.) and then treated with T4DNA polymerase (produced by TAKARA SHUZO CO., LTD.) to form a blunt terminal, whereby obtaining a DNA fragment.

On the other hand, pGFP-C1 (produced by Toyobo Co., Ltd.) was digested with Bsu36I (NEB) and then treated with T4DNA polymerase (produced by TAKARA SHUZO CO., LTD.) to form a blunt terminal, whereby obtaining a 1.6 kb of a DNA fragment. The both DNA fragments were ligated to construct a reporter plasmid pGL3-4ERPP-TK neo.

### Reference Example 6a

(Human PPARγ Gene Cloning)

A human PPARγ gene was cloned using a heart cDNA (produced by Toyobo Co., Ltd., trade name: QUICK-Clone cDNA) as a template by means of a PCR method employing a primer set shown below which was prepared with reference to the base sequence of PPARγ gene reported by Greene et al (Gene Expr., 1995, Vol. 4 (4-5), page 281-299).

PAG-U:

(SEQ ID NO:9)

5′-GTG GGT ACC GAA ATG ACC ATG GTT GAC ACA GAG-3′
PAG-L:

(SEQ ID NQ:10)

5′-GGG GTC GAC CAG GAC TCT CTG CTA GTA CAA GTC-3′

The PCR reaction was performed by Hot Start method using AmpliWax PCR Gem 100 (produced by TAKARA SHUZO CO., LTD.). First, 2 μl of 10×LA PCR Buffer, 3 μl of 2.5 mM dNTP solution, 2.5 μl each of 12.5 μM primer solutions and 10 μl of sterilized distilled water were mixed to obtain a bottom layer solution mixture. 1 μl of human heart cDNA (1 ng/ml) as a template, 3 μl of 10×LA PCR Buffer, 1 μl of 2.5 mM dNTP solution, 0.5 μl of TaKaRa LA Taq DNA polymerase (produced by TAKARA SHUZO CO., LTD.) and 24.5 μl of sterilized distilled water were mixed to obtain a top layer solution mixture.

To the bottom layer solution mixture described above, added was one unit of AmpliWax PCR Gem 100 (produced by TAKARA SHUZO CO., LTD.), which was treated at 70° C. for 5 minutes and then in ice for 5 minutes. Then the top layer solution mixture was added to the mixture to prepare the reaction mixture of PCR. A tube containing the reaction mixture was set on a thermal cycler (produced by Perkin Elmer, USA) and treated at 95° C. for 2 minutes. After repeating the cycle of 95° C. for 15 seconds and 68° C. for 2 minutes a further 35 times, the tube was treated at 72° C. for 8 minutes.

The PCR product thus obtained was subjected to electrophoresis on agarose gel (1%), and 1.4 kb DNA fragment containing PPARγ gene was recovered from the gel, and then inserted into pT7 Blue-T vector (produced by TAKARA SHUZO CO., LTD.) to obtain a plasmid pTBT-hPPARγ.

### Reference Example 7a

(Construction of Plasmids for Expressing Human PPARγ, RXRα)

A 7.8 kb FspI-NotI fragment of plasmid pVgRXR (produced by Invitrogen, USA) was ligated to a 0.9 kb FspI-NotI fragment containing RXRα gene of plasmid pTBT-hRXRα obtained in Reference Example 2a to prepare plasmid pVgRXR2. Then, pVgRXR2 was digested with BstXI and then treated with T4DNA polymerase (produced by TAKARA SHUZO CO., LTD.) to obtain a blunt terminal. Then digestion at KpnI gave a 6.5 kb DNA fragment. On the other hand, plasmid pTBT-hPPARγ obtained in Reference Example 6a was digested with Sal I and then treated with T4DNA polymerase (produced by TAKARA SHUZO CO., LTD.) to obtain a blunt terminal. Then digestion at KpnI gave a 1.4 kb DNA fragment containing human PPARγ gene.

The both DNA fragments were ligated to construct plasmid pVgRXR2-hPPARγ.

### Reference Example 8a

(Introduction of Plasmids for Expressing Human PPARγ and RXRα, and Reporter Plasmid into CHO-K1 Cell and Establishment of Expressed Cell)

After a CHO-K1 cell cultured in a 150 cm² cell culture flask (750 ml)(produced by Corning Costar Corporation, USA) containing HAM F12 medium (produced by Life Technologies, Inc., USA) supplemented with 10% Fetal Bovine Serum (produced by Life Technologies, Inc., USA) was scraped by treating with 0.5 g/L trypsin-0.2 g/L EDTA (ethylenediaminetetraacetic acid) (produced by Life Technologies, Inc., USA), the cell was washed with PBS (phosphate-buffered saline) (produced by Life Technologies, Inc., USA), centrifuged (1000 rpm, 5 minutes), and then suspended in PBS. Subsequently, a DNA was introduced into the cell under the condition shown below using GENE PULSER (produced by Bio-Rad Laboratories, USA).

Namely, to a cuvette having a 0.4 cm gap, added were 8×10⁶ cells and 10 μg of plasmid pVgRXR2-hPPARγ obtained in Reference Example 7a and 10 μg of reporter plasmid pGL3-4ERPP-TK neo obtained in Reference Example 5a, which was subjected to electroporation at the voltage of 0.25 kV under the capacitance of 960 μF. Subsequently, the cell was transferred into a HAM F12 medium containing 10% Fetal Bovine Serum and cultured for 24 hours and then the cell was scraped again and centrifuged, and then suspended in HAM F12 medium containing 10% Fetal Bovine Serum supplemented with 500 μg/ml of GENETICIN (produced by Life Technologies, Inc., USA) and 250 μg/ml of ZEOCIN (produced by Invitrogen, USA). The obtained suspension was diluted to the density of 10⁴ cells/ml and inoculated to a 96-well plate (produced by Corning Costar Corporation, USA), which was cultured in a CO₂ gas incubator at 37° C., whereby obtaining a GENETICIN- and ZEOCIN-resistant transformant.

Subsequently, after the transformant cell line thus obtained was cultured in a 24-well plate (produced by Corning Costar Corporation, USA), selected was a cell line in which the luciferase was expressed and induced, i.e., PPARγ:RXRα:4ERPP/CHO-K1 cell by addition of 10 μM of pioglitazone hydrochloride.

### Reference Example 9a

(Introduction of Plasmids for Expressing Human PPARδ and RXRα, and Reporter Plasmid into COS-1 Cell and Establishment of Transformant)

COS-1 cells were inoculated to a 150 cm² cell culture flask (produced by Corning Costar Corporation, USA) at the density of 5×10⁶ cells/50 ml, and cultured at 37° C. under 5% CO₂ conditions for 24 hours. Subsequently, a DNA was introduced into the cell under the condition shown below using Lipofectamine (produced by Invitrogen, USA).

First, Lipofectamine (125 μl), PLUS Reagent (100 μl, produced by Invitrogen, USA), plasmid pMCMVneo-hPPARδ (2.5 μg) obtained in Reference Example 3a, plasmid pMCMVneo-hRXRα (2.5 μg) obtained in Reference Example 4a and reporter plasmid pGL3-4ERPP-TK neo (5 μg) obtained in Reference Example 5a, and pRL-tk (5 μg, produced by Promega, USA) were mixed with opti-MEM (5 ml, produced by Invitrogen, USA) to give a transfection mixture.

Then, the above-mentioned transfection mixture and opti-MEM (20 ml) were added to COS-1 cells washed with opti-MEM, and the cells were cultured at 37° C. under 5% CO₂ conditions for 3 hours. DMEM medium (25 ml, produced by Life Technologies, Inc., USA) containing 0.1% fatty acid-free bovine serum albumin (BSA) (produced by Wako Pure Chemical Industries, Ltd.) was added to the obtained COS-1 cells, and the cells were cultured at 37° C. under 5% CO₂ conditions for 18-24 hours to give a transformant.

### Reference Example 10a

### Construction of Expression Vector for Human GPR40

The DNA fragment encoding human GPR40 was obtained by the following PCR method. That is, a mixture (50 μl) was prepared containing 20 pmol each of an oligo DNA (SEQ ID NO:11) depicted by 5′>CGTCGACCCGGCGGCCCCATGGACCTGCCCCCG<3′ as a sense chain primer and an oligo DNA (SEQ ID NO:12) depicted by 5′>CATCGATTAGCAGTGGCGTTACTTCTGGGACTT<3′ as an antisense chain primer, 5 μl of 10× Advantage (trademark) 2 PCR Buffer (CLONTECH), 1 μl of 50×dNTP mix (CLONTECH), 1 μl of 50× Advantage 2 Polymerase Mix (CLONTECH) and 1 μl of human pancreatic cDNA (CLONTECH) as a template DNA. PCR was performed using a thermal cycler (GeneAmp (trademark) PCR system model 9700 (Applied Biosystems)), and repeating 35 cycles of 96° C., 1 min, then 96° C., 30 sec→+61° C., 30 sec→72° C., 120 sec, followed by elongation at 72° C. for 10 min. The resulting reaction mixture was applied to agarose gel electrophoresis to give a single product, cloned using a TA cloning kit (Invitrogen), and the gene sequence was confirmed. The clones free of PCR error were digested twice with restriction enzymes SalI (Takara Shuzo) and ClaI (Takara Shuzo) and applied to agarose gel electrophoresis, upon which a single product was cleaved out. The obtained fragment (ca. 1 kb) was introduced into a pAKKO-111 vector, which was used for transfection of CHO cells.

### Reference Example 1

To a mixture of N-hydroxy-4-(trifluoromethyl)benzenecarboximidoyl chloride (11.00 g), 4-pentyn-1-ol (4.98 g) and tetrahydrofuran (150 ml) was dropwise added a solution (10 ml) of triethylamine (10 ml) in tetrahydrofuran at 0° C. and the mixture was stirred at room temperature overnight. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and 3-{3-[4-(trifluoromethyl)phenyl]-5-isoxazolyl}-1-propanol (10.68 g, yield 80%) was obtained as colorless crystals from a fraction eluted with ethyl acetate-hexane (1:2, volume ratio). The crystals were recrystallized from ethyl acetate-hexane. melting point: 59-60° C.

¹H-NMR (CDCl₃)δ: 1.41 (1H, br t), 1.92-2.14 (2H, m), 2.88-3.05 (2H, m), 3.68-3.86 (2H, m), 6.37 (1H, s), 7.66-7.76 (2H, m), 7.87-7.97 (2H, m).

### Reference Example 2

To a mixture of 3-{3-[4-(trifluoromethyl)phenyl]-5-isoxazolyl)-1-propanol (9.68 g), triethylamine (6.5 ml) and ethyl acetate (150 ml), was dropwise added a solution (10 ml) of methanesulfonyl chloride (3.3 ml) in ethyl acetate at 0° C. and the mixture was stirred at room temperature overnight. The reaction mixture was poured into dilute hydrochloric acid, and extracted with ethyl acetate. The ethyl acetate layer was washed with saturated aqueous sodium hydrogen carbonate and then saturated aqueous sodium chloride solution, dried (MgSO₄) and concentrated. The residue was subjected to silica gel column chromatography, and 3-{3-[4-(trifluoromethyl)phenyl]-5-isoxazolyl}-1-propyl methanesulfonate (11.78 g, yield 94%) was obtained as a pale-yellow oily substance from a fraction eluted with ethyl acetate-hexane (1:2, volume ratio).

¹H-NMR (CDCl₃)δ: 1.96-2.10 (2H, m), 2.86-2.96 (2H, m), 3.16 (3H, s), 4.24-4.34 (2H, m), 6.36 (1H, s), 7.65-7.76 (2H, m), 7.86-7.97 (2H, m).

### Reference Example 3

A mixture of 3-hydroxy-1-phenyl-1H-pyrazole-5-carboxylic acid (29.55 g), benzyl bromide (35 ml), potassium carbonate (40.99 g) and N,N-dimethylformamide (300 ml) was stirred overnight at 90° C. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and benzyl 3-benzyloxy-1-phenyl-1H-pyrazole-5-carboxylate (51.33 g, yield 92%) was obtained as a colorless oil from a fraction eluted with ethyl acetate-hexane (1:2, volume ratio).

¹H-NMR (CDCl₃)δ: 5.20 (2H, s), 5.27 (2H, s), 6.49 (1H, s), 7.18-7.47 (15H, m).

### Reference Example 4

A mixture of benzyl 3-benzyloxy-1-phenyl-1H-pyrazole-5-carboxylate (50.88 g), 1N aqueous sodium hydroxide solution (200 ml), tetrahydrofuran (200 ml) and ethanol (200 ml) was refluxed at room temperature for 5 hours. 1N Hydrochloric acid (200 ml) was added and the mixture was extracted with ethyl acetate. The ethyl acetate layer was washed with saturated aqueous sodium chloride solution, dried (MgSO₄) and concentrated. The obtained colorless crystals were collected by filtration to give 3-benzyloxy-1-phenyl-1H-pyrazole-5-carboxylic acid (36.91 g, yield 95%). The crystals were recrystallized from acetone-isopropyl ether. melting point: 163-164° C.

¹H-NMR (CDCl₃)δ: 5.27 (2H, s), 6.52 (1H, s), 7.30-7.50 (10H, m).

### Reference Example 5

A mixture of 3-benzyloxy-1-phenyl-1H-pyrazole-5-carboxylic acid (33.00 g), iodomethane (8.5 ml), potassium carbonate (18.88 g) and N,N-dimethylformamide (300 ml) was stirred at room temperature overnight. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and methyl 3-benzyloxy-1-phenyl-1H-pyrazole-5-carboxylate (33.48 g, yield 97%) was obtained as colorless crystals from a fraction eluted with ethyl acetate-hexane (1:4, volume ratio). The crystals were recrystallized from ethyl acetate-hexane. melting point: 53-54° C.

¹H-NMR (CDCl₃)δ: 3.77 (3H, s), 5.28 (2H, s), 6.44 (1H, s), 7.32-7.49 (10H, m).

### Reference Example 6

A mixture of methyl 3-benzyloxy-1-phenyl-1H-pyrazole-5-carboxylate (15.00 g), 5% palladium-carbon (10.92 g) and tetrahydrofuran (200 ml) was stirred at room temperature for 1 hour under a hydrogen atmosphere. Palladium-carbon was removed by filtration and the filtrate was concentrated. The residue was subjected to silica gel column chromatography, and methyl 3-hydroxy-1-phenyl-1H-pyrazole-5-carboxylate (10.30 g, yield 97%) was obtained as colorless crystals from a fraction eluted with ethyl acetate-hexane (1:2, volume ratio). The crystals were recrystallized from tetrahydrofuran-isopropyl ether. melting point: 227-228° C.

¹H-NMR (CDCl₃)δ: 3.77 (3H, s), 6.32 (1H, s), 7.35-7.54 (5H, m), 10.77 (1H, br s).

### Reference Example 7

To a mixture of methyl 3-benzyloxy-1-phenyl-1H-pyrazole-5-carboxylate (14.53 g) and tetrahydrofuran (300 ml) was slowly added lithium aluminum hydride (1.79 g) at 0° C. and the mixture was stirred at room temperature for 1 hour. To the reaction mixture was slowly added sodium sulfate 10 hydrate (15.20 g) at 0° C. and the mixture was stirred at room temperature for 30 minutes. The insoluble material was removed by filtration and the filtrate was concentrated. The residue was subjected to silica gel column chromatography, and (3-benzyloxy-1-phenyl-1H-pyrazol-5-yl)methanol (11.65 g, yield 88%) was obtained as colorless crystals from a fraction eluted with ethyl acetate-hexane (1:2, volume ratio). The crystals were recrystallized from ethyl acetate-hexane. melting point: 87-88° C.

¹H-NMR (CDCl₃)δ: 1.79 (1H, t, J=6.0 Hz), 4.61 (2H, d, J=6.0 Hz), 5.28 (2H, s), 5.94 (1H, s), 7.30-7.60 (10H, m).

### Reference Example 8

A mixture of (3-benzyloxy-1-phenyl-1H-pyrazol-5-yl)methanol (11.20 g), activated manganese dioxide (30.00 g) and tetrahydrofuran (300 ml), was stirred overnight at room temperature. The insoluble material was removed by filtration and the filtrate was concentrated. The residue was subjected to silica gel column chromatography, and 3-benzyloxy-1-phenyl-1H-pyrazole-5-carbaldehyde (10.10 g, yield 91%) was obtained as a pale-yellow oily substance from a fraction eluted with ethyl acetate-hexane (1:2, volume ratio).

¹H-NMR (CDCl₃)δ: 5.31 (2H, s), 6.51 (1H, s), 7.32-7.52 (10H, m), 9.78 (1H, s).

### Reference Example 9

To a mixture of 3-benzyloxy-1-phenyl-1H-pyrazole-5-carbaldehyde (6.24 g), ethyl diethylphosphonoacetate (5.55 g) and N,N-dimethylformamide (50 ml) was added sodium hydride (60%, in oil, 960 mg) at 0° C. and the mixture was stirred overnight at room temperature. The reaction mixture was poured into water, and the mixture was extracted with ethyl acetate. The ethyl acetate layer was washed with dilute hydrochloric acid and then with saturated aqueous sodium chloride solution, dried (MgSO₄) and concentrated. The residue was subjected to silica gel column chromatography, and ethyl (E)-3-(3-benzyloxy-1-phenyl-1H-pyrazol-5-yl)propenoate (7.33 g, yield 94%) was obtained as a pale-yellow oily substance from a fraction eluted with ethyl acetate-hexane (1:4, volume ratio).

¹H-NMR (CDCl₃)δ: 1.30 (3H, t, J=6.8 Hz), 4.23 (2H, q, J=6.8 Hz), 5.29 (2H s), 6.18 (1H, s), 6.33 (1H, d, J=15.8 Hz), 7.28-7.55 (10H, m).

### Reference Example 10

A mixture of ethyl (E)-3-(3-benzyloxy-1-phenyl-1H-pyrazol-5-yl)propenoate (7.33 g), 5% palladium-carbon (7.11 g) and tetrahydrofuran (50 ml) was stirred overnight at room temperature under a hydrogen atmosphere. Palladium-carbon was removed by filtration and the filtrate was concentrated. The residue was subjected to silica gel column chromatography, and ethyl 3-(3-hydroxy-1-phenyl-1H-pyrazol-5-yl)propionate (4.85 g, yield 89%) was obtained as colorless crystals from a fraction eluted with ethyl acetate-hexane (1:2, volume ratio). The crystals were recrystallized from acetone-hexane. melting point: 150-151° C.

¹H-NMR (CDCl₃)δ: 1.23 (3H, t, J=7.2 Hz), 2.52-2.60 (2H, m) 2.86-2.94 (2H, m), 4.11 (2H, q, J=7.2 Hz), 5.59 (1H, s), 7.33-7.51 (5H, m).

### Reference Example 11

A mixture of methyl 3-hydroxy-1-methyl-1H-pyrazole-5-carboxylate (1.45 g), benzyl bromide (1.16 ml), potassium carbonate (1.54 g) and N,N-dimethylformamide (10 ml) was stirred at room temperature for 2 hours. The reaction mixture was poured into dilute hydrochloric acid, and extracted with ethyl acetate; The ethyl acetate layer was washed with saturated aqueous sodium chloride solution, dried (MgSO₄) and concentrated. The residue was subjected to silica gel column chromatography, and methyl 3-benzyloxy-1-methyl-1H-pyrazole-5-carboxylate (2.20 g, yield 96%) was obtained as a colorless oil from a fraction eluted with ethyl acetate-hexane (1:5, volume ratio).

¹H-NMR (CDCl₃)δ: 3.86 (3H, s), 4.05 (3H, s), 5.19 (2H, s), 6.21 (1H, s), 7.27-7.50 (5H, m).

### Reference Example 12

To a mixture of methyl 3-benzyloxy-1-methyl-1H-pyrazole-5-carboxylate (9.60 g) and tetrahydrofuran (100 ml) was slowly added lithium aluminum hydride (890 mg) at 0° C. and the mixture was stirred at room temperature for 1 hour. To the reaction mixture was slowly added sodium sulfate 10 hydrate (8.43 g) at 0° C., and the mixture was stirred at room temperature for 1 hour. The insoluble material was removed by filtration and the filtrate was concentrated. The residue was subjected to silica gel column chromatography, and (3-benzyloxy-1-methyl-1H-pyrazol-5-yl)methanol (8.52 g, quantitative) was obtained as a pale-yellow oily substance from a fraction eluted with ethyl acetate-hexane (1:2, volume ratio).

¹H-NMR (CDCl₃)δ: 1.72 (1H, br s), 3.76 (3H, s), 4.58 (2H, d, J=6.2 Hz), 5.16 (2H, s), 5.64 (1H, s), 7.27-7.50 (5H, m).

### Reference Example 13

A mixture of (3-benzyloxy-1-methyl-1H-pyrazol-5-yl)methanol (9.40 g), activated manganese dioxide (29.10 g) and tetrahydrofuran (200 ml) was stirred overnight at room temperature. The insoluble material was removed by filtration and the filtrate was concentrated. The residue was subjected to silica gel column chromatography, and 3-benzyloxy-1-methyl-1H-pyrazole-5-carbaldehyde (6.05 g, yield 65%) was obtained as colorless crystals from a fraction eluted with ethyl acetate-hexane (1:3, volume ratio). The crystals were recrystallized from ethyl acetate-hexane. melting point: 49.5-50.5° C.

¹H-NMR (CDCl₃)δ: 4.05 (3H, s), 5.22 (2H, s), 6.25 (1H, s), 7.26-7.51 (5H, m), 9.73 (1H, s).

### Reference Example 14

To a mixture of 3-benzyloxy-1-methyl-1H-pyrazole-5-carbaldehyde (3.05 g), ethyl diethylphosphonoacetate (3.25 g) and N,N-dimethylformamide (50 ml) was added sodium hydride (60%, in oil, 575 mg) at 0° C., and the mixture was stirred overnight at room temperature. The reaction mixture was poured into water, and extracted with ethyl acetate. The ethyl acetate layer was washed with dilute hydrochloric acid and then with saturated aqueous sodium chloride solution, dried (MgSO₄) and concentrated. The residue was subjected to silica gel column chromatography, and ethyl (E)-3-(3-benzyloxy-1-methyl-1H-pyrazol-5-yl)propenoate (3.34 g, yield 83%) was obtained as a colorless oil from a fraction eluted with ethyl acetate-hexane (1:4, volume ratio).

¹H-NMR (CDCl₃)δ: 1.33 (3H, t, J=7.0 Hz), 3.82 (3H, s), 4.26 (2H, q, J=7.0 Hz), 5.18 (2H, s), 5.95 (1H, s), 6.27 (1H, d, J=15.8 Hz), 7.27-7.53 (6H, m).

### Reference Example 15

A mixture of ethyl (E)-3-(3-benzyloxy-1-methyl-1H-pyrazol-5-yl)propenoate (730 mg), 10% palladium-carbon (73 mg) and methanol (15 ml) was stirred at room temperature for 1 hour under a hydrogen atmosphere. Palladium-carbon was removed by filtration and the filtrate was concentrated. The obtained colorless crystals were collected by filtration to give ethyl 3-(3-hydroxy-1-methyl-1H-pyrazol-5-yl)propionate (440 mg, yield 87%). The crystals were recrystallized from ethyl acetate-hexane. melting point: 132-135° C.

¹H-NMR (CDCl₃)δ: 1.26 (3H, t, J=6.9 Hz), 2.59-2.66 (2H, m), 2.80-2.87 (2H, m), 3.61 (3H, s), 4.15 (2H, q, J=6.9 Hz), 5.39 (1H, s).

### Reference Example 16

A mixture of ethyl 3-methyl-1H-pyrazole-4-carboxylate (23.10 g), 2-chloro-5-(trifluoromethyl)pyridine (25.09 g), potassium carbonate (19.00 g) and N,N-dimethylformamide (300 ml) was stirred overnight at 100° C. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and ethyl 3-methyl-1-[5-(trifluoromethyl)-2-pyridyl]-1H-pyrazole-4-carboxylate (40.22 g, yield 97%) was obtained as colorless crystals from a fraction eluted with ethyl acetate-hexane (1:4, volume ratio). The crystals were recrystallized from ethyl acetate-hexane. melting point: 88-89° C.

¹H-NMR (CDCl₃)δ: 1.38 (3H, t, J=7.2 Hz), 2.57 (3H, s), 4.34 (2H, q, J=7.2 Hz), 8.05 (1H, dd, J=2.4, 9.3 Hz), 8.10 (1H, d, J=9.3 Hz), 8.64-8.72 (1H, m), 9.00 (1H, s).

### Reference Example 17

To a solution of ethyl 3-methyl-1-[5-(trifluoromethyl)-2-pyridyl]-1H-pyrazole-4-carboxylate (35.19 g) in tetrahydrofuran (300 ml) was dropwise added a 1.0 M solution (360 ml) of diisobutylaluminum hydride in hexane at 0° C., and the mixture was stirred at room temperature for 1 hour. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and {3-methyl-1-[5-(trifluoromethyl)-2-pyridyl]-1H-pyrazol-4-yl}methanol (29.33 g, yield 97%) was obtained as colorless crystals from a fraction eluted with ethyl acetate-hexane (1:1, volume ratio). The crystals were recrystallized from ethyl acetate-hexane. melting point: 157-158° C.

¹H-NMR (CDCl₃)δ: 1.46 (1H, t, J=5.4 Hz), 2.39 (3H, s), 4.64 (2H, d, J=5.4 Hz), 7.98-8.04 (2H, m), 8.49 (1H, s), 8.60-8.66 (1H, m).

### Reference Example 18

To a mixture of N-hydroxy-4-(trifluoromethyl)benzenecarboximidoyl chloride (13.11 g), 5-hexyn-1-ol (5.88 g) and tetrahydrofuran (300 ml) was dropwise added a solution (50 ml) of triethylamine (17 ml) in tetrahydrofuran at 0° C., and the mixture was stirred at room temperature overnight. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and 4-{3-[4-(trifluoromethyl)phenyl]-5-isoxazolyl}-1-butanol (13.92 g, yield 83%) was obtained as colorless crystals from a fraction eluted with ethyl acetate-hexane (1:2, volume ratio). The crystals were recrystallized from ethyl acetate-hexane. melting point: 68-69° C.

¹H-NMR (CDCl₃)δ: 1.60-1.98 (4H, m), 2.80-2.95 (2H, m), 3.66-3.78 (2H, m), 6.36 (1H, s), 7.66-7.76 (2H, m), 7.86-7.96 (2H, m).

### Reference Example 19

To a mixture of 4-{3-[4-(trifluoromethyl)phenyl]-5-isoxazolyl}-1-butanol (7.00 g), triethylamine (4 ml) and ethyl acetate (180 ml), was dropwise added a solution (20 ml) of methanesulfonyl chloride (2 ml) in ethyl acetate at 0° C., and the mixture was stirred at room temperature overnight. The reaction mixture was poured into dilute hydrochloric acid, and extracted with ethyl acetate. The ethyl acetate layer was washed with saturated aqueous sodium hydrogen carbonate and then saturated aqueous sodium chloride solution, dried (MgSO₄) and concentrated. The residue was subjected to silica gel column chromatography, and 4-(3-[4-(trifluoromethyl)phenyl]-5-isoxazolyl}-1-butyl methanesulfonate (8.42 g, yield 95%) was obtained as a pale-yellow oily substance from a fraction eluted with ethyl acetate-hexane (1:2, volume ratio).

¹H-NMR (CDCl₃)δ: 1.78-2.04 (4H, m), 2.82-2.94 (2H, m), 3.14 (3H, s), 4.22-4.34 (2H, m), 6.36 (1H, s), 7.65-7.76 (2H, m), 7.86-7.97 (2H, m).

### Reference Example 20

A mixture of ethyl 3-isopropyl-1H-pyrazole-4-carboxylate (5.00 g), 2-chloro-5-(trifluoromethyl)pyridine (4.95 g), potassium carbonate (3.80 g) and N,N-dimethylformamide (50 ml) was stirred overnight at 100° C. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and ethyl 3-isopropyl-1-[5-(trifluoromethyl)-2-pyridyl]-1H-pyrazole-4-carboxylate (8.61 g, yield 96%) was obtained as colorless crystals from a fraction eluted with ethyl acetate-hexane (1:4, volume ratio). The crystals were recrystallized from ethyl acetate-hexane. melting point: 94-95° C.

¹H-NMR (CDCl₃)δ: 1.32-1.44 (9H, m), 3.52-3.68 (1H, m), 4.33 (2H, q, J=7.0 Hz), 8.03 (1H, dd, J=2.2, 8.8 Hz), 8.14 (1H, d, J=8.8 Hz), 8.68 (1H, d, J=2.2 Hz), 8.98 (1H, s).

### Reference Example 21

To a solution of ethyl 3-isopropyl-1-[5-(trifluoromethyl)-2-pyridyl]-1H-pyrazole-4-carboxylate (8.50 g) in tetrahydrofuran (200 ml) was dropwise added a 1.0 M solution (60 ml) of diisobutylaluminum hydride in hexane at 0° C., and the mixture was stirred at room temperature for 1 hour. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and (3-isopropyl-1-[5-(trifluoromethyl)-2-pyridyl]-1H-pyrazol-4-yl]methanol (7.20 g, yield 97%) was obtained as colorless crystals from a fraction eluted with ethyl acetate-hexane (1:1, volume ratio). The crystals were recrystallized from ethyl acetate-hexane. melting point: 119-120° C.

¹H-NMR (CDCl₃)δ:1.36 (6H, d, J=6.8 Hz), 1.45 (1H, t, J=5.6 Hz), 3.05-3.24 (1H, m), 4.67 (2H, d, J=5.6 Hz), 7.92-8.10 (2H, m), 8.49 (1H, s), 8.59-8.67 (1H, m).

### Reference Example 22

A mixture of {3-isopropyl-1-[5-(trifluoromethyl)-2-pyridyl]-1H-pyrazol-4-yl}methanol (5.85 g), activated manganese dioxide (15.44 g) and tetrahydrofuran (300 ml) was stirred overnight at room temperature. The insoluble material was removed by filtration and the filtrate was concentrated. The residue was subjected to silica gel column chromatography, and 3-isopropyl-1-[5-(trifluoromethyl)-2-pyridyl]-1H-pyrazole-4-carbaldehyde (5.22 g, yield 90%) was obtained as colorless crystals from a fraction eluted with ethyl acetate-hexane (1:4, volume ratio). The crystals were recrystallized from ethyl acetate-hexane. melting point: 89-90° C.

¹H-NMR (CDCl₃)δ: 1.38 (6H, d, J=7.0 Hz), 3.42-3.59 (1H, m), 8.06 (1H, dd, J=2.2, 8.4 Hz), 8.15 (1H, d, J=8.4 Hz), 8.70 (1H, d, J=2.2 Hz), 9.04 (1H, s), 10.06 (1H, s).

### Reference Example 23

To a mixture of 3-isopropyl-1-[5-(trifluoromethyl)-2-pyridyl]-1H-pyrazole-4-carbaldehyde (5.00 g), ethyl diethylphosphonoacetate (4.05 g) and N,N-dimethylformamide (50 ml) was added sodium hydride (60%, in oil, 730 mg) at 0° C. and the mixture was stirred overnight at room temperature. The reaction mixture was poured into water, and extracted with ethyl acetate. The ethyl acetate layer was washed with dilute hydrochloric acid and then with saturated aqueous sodium chloride solution, dried (MgSO₄) and concentrated. The residue was subjected to silica gel column chromatography, and ethyl (E)-3-{3-isopropyl-1-[5-(trifluoromethyl)-2-pyridyl]-1H-pyrazol-4-yl]propenoate (5.93 g, yield 95%) was obtained as colorless crystals from a fraction eluted with ethyl acetate-hexane (1:4, volume ratio). The crystals were recrystallized from ethyl acetate-hexane. melting point: 112-113° C.

¹H-NMR (CDCl₃)δ: 1.34 (3H, t, J=7.4 Hz), 1.37 (6H, d, J=7.0 Hz), 3.14-3.32 (1H, m), 4.26 (2H, q, J=7.4 Hz), 6.29 (1H, d, J=16.0 Hz), 7.63 (1H, d, J=16.0 Hz), 7.96-8.15 (2H, m), 8.63-8.69 (1H, m), 8.75 (1H, s).

### Reference Example 24

A mixture of ethyl (E)-3-(3-isopropyl-1-[5-(trifluoromethyl)-2-pyridyl]-1H-pyrazol-4-yl}propenoate (5.80 g), 5% palladium-carbon (1.35 g) and tetrahydrofuran (50 ml) was stirred at room temperature for 1 hour under a hydrogen atmosphere. Palladium-carbon was removed by filtration and the filtrate was concentrated. The residue was subjected to silica gel column chromatography, and ethyl 3-{3-isopropyl-1-[5-(trifluoromethyl)-2-pyridyl]-1H-pyrazol-4-yl}propionate (5.82 g, quantitative) was obtained as a colorless oil from a fraction eluted with ethyl acetate-hexane (1:2, volume ratio).

¹H-NMR (CDCl₃)δ: 1.27 (3H, t, J=7.0 Hz), 1.33 (6H, d, J=7.0 Hz), 2.58-3.16 (5H, m), 4.16 (2H, q, J=7.0 Hz), 7.90-8.06 (2H, m), 8.26-8.33 (1H, m), 8.56-8.64 (1H, m).

### Reference Example 25

To a solution of ethyl 3-{3-isopropyl-1-[5-(trifluoromethyl)-2-pyridyl]-1H-pyrazol-4-yl)propionate (5.82 g) in tetrahydrofuran (50 ml) was dropwise added a 1.0 M solution (40 ml) of diisobutylaluminum hydride in hexane at 0° C., and the mixture was stirred at room temperature for 1 hour. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and 3-{3-isopropyl-1-[5-(trifluoromethyl)-2-pyridyl]-1H-pyrazol-4-yl}-1-propanol (4.50 g, yield 88%) was obtained as colorless crystals from a fraction eluted with ethyl acetate-hexane (1:1, volume ratio). The crystals were recrystallized from ethyl acetate-hexane. melting point: 87-88° C.

¹H-NMR (CDCl₃)δ: 1.33 (6H, d, J=7.0 Hz), 1.82-2.02 (2H, m), 2.53-2.68 (2H, m), 2.95-3.16 (1H, m), 3.68-3.84 (2H, m), 7.90-8.08 (2H, m), 8.28 (1H, s), 8.57-8.64 (1H, m).

### Reference Example 26

To a solution of methyl 3-(3-isopropyl-1-[5-(trifluoromethyl)-2-pyridyl]-1H-pyrazol-4-ylmethoxy}-1-methyl-1H-pyrazole-5-carboxylate (1.90 g) in tetrahydrofuran (30 ml) was dropwise added a 1.0 M solution (15 ml) of diisobutylaluminum hydride in hexane at 0° C., and the mixture was stirred at room temperature for 1 hour. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and (3-{3-isopropyl-1-[5-(trifluoromethyl)-2-pyridyl]-1H-pyrazol-4-ylmethoxy}-1-methyl-1H-pyrazol-5-yl)methanol (1.70 g, yield 96%) was obtained as a colorless oil from a fraction eluted with ethyl acetate-hexane (1:1, volume ratio).

¹H-NMR (CDCl₃)δ: 1.36 (6H, d, J=7.0 Hz), 3.04-3.27 (1H, m), 3.78 (3H, s), 4.59 (2H, s), 5.13 (2H, s), 5.64 (1H, s), 7.97 (1H, dd, J=2.2, 8.8 Hz), 8.06 (1H, d, J=8.8 Hz), 8.56 (1H, s), 8.60-8.64 (1H, m).

### Reference Example 27

A mixture of (3-{3-isopropyl-1-[5-(trifluoromethyl)-2-pyridyl]-1H-pyrazol-4-ylmethoxy}-1-methyl-1H-pyrazol-5-yl)methanol (1.70 g), activated manganese dioxide (5.11 g) and tetrahydrofuran (50 ml) was stirred overnight at room temperature. The insoluble material was removed by filtration and the filtrate was concentrated. The residue was subjected to silica gel column chromatography, and 3-{3-isopropyl-1-[5-(trifluoromethyl)-2-pyridyl]-1H-pyrazol-4-ylmethoxy}-1-methyl-1H-pyrazole-5-carbaldehyde (1.41 g, yield 83%) was obtained as colorless crystals from a fraction eluted with ethyl acetate-hexane (1:4, volume ratio). The crystals were recrystallized from ethyl acetate-hexane. melting point: 112-113° C.

¹H-NMR (CDCl₃)δ: 1.37 (6H, d, J=6.8 Hz), 3.07-3.25 (1H, m), 4.06 (3H, s), 5.18 (2H, s), 6.25 (1H, s), 7.98 (1H, dd, J=2.2, 8.4 Hz), 8.07 (1H, d, J=8.4 Hz), 8.58 (1H, s), 8.60-8.65 (1H, m), 9.75 (1H, s).

### Reference Example 28

A mixture of ethyl 3-(3-ethoxy-1H-pyrazol-4-yl)propionate (12.98 g), 2-chloro-5-(trifluoromethyl)pyridine (11.10 g), potassium carbonate (12.33 g) and N,N-dimethylformamide (150 ml) was stirred overnight at 100° C. The reaction mixture was poured into dilute hydrochloric acid, and extracted with ethyl acetate. The ethyl acetate layer was washed with saturated aqueous sodium chloride solution, dried (MgSO₄) and concentrated. To a solution of the residue in tetrahydrofuran (200 ml) was dropwise added a 1.0 M solution (140 ml) of diisobutylaluminum hydride in hexane at 0° C., and the mixture was stirred at room temperature for 1 hour. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and 3-{3-ethoxy-1-[5-(trifluoromethyl)-2-pyridyl]-1H-pyrazol-4-yl}-1-propanol (6.10 g, yield 32%) was obtained as colorless crystals from a fraction eluted with ethyl acetate-hexane (1:1, volume ratio). The crystals were recrystallized from ethyl acetate-hexane. melting point: 85-86° C.

¹H-NMR (CDCl₃)δ: 1.44 (3H, t, J=7.2 Hz), 1.65 (1H, br t), 1.80-1.94 (2H, m), 2.54 (2H, t, J-7.2 Hz), 3.64-3.78 (2H, m), 4.38 (2H, q, J=7.2 Hz), 7.82 (1H, d, J=8.7 Hz), 7.91 (1H, dd, J=2.4, 8.7 Hz), 8.19 (1H, s), 8.53-8.59 (1H, m).

### Reference Example 29

To a solution of methyl 1-methyl-3-{3-methyl-1-[5-(trifluoromethyl)-2-pyridyl]-1H-pyrazol-4-ylmethoxy}-1H-pyrazole-5-carboxylate (4.74 g) in tetrahydrofuran (30 ml) was dropwise added a 1.0 M solution (30 ml) of diisobutylaluminum hydride in hexane at 0° C., and the mixture was stirred at room temperature for 1 hour. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and (1-methyl-3-{3-methyl-1-[5-(trifluoromethyl)-2-pyridyl]-1H-pyrazol-4-ylmethoxy}-1H-pyrazol-5-yl)methanol (4.18 g, yield 88%) was obtained as colorless crystals from a fraction eluted with ethyl acetate-hexane (1:1, volume ratio). The crystals were recrystallized from ethyl acetate-hexane. melting point: 128-129° C.

¹H-NMR (CDCl₃)δ: 1.58 (1H, t, J=5.7 Hz), 2.40 (3H, s), 3.77 (3H, s), 4.59 (2H, d, J=5.7 Hz), 5.10 (2H, s), 5.63 (1H, s), 7.94-8.06 (2H, m), 8.56 (1H, s), 8.58-8.67 (1H, m).

### Reference Example 30

A mixture of (1-methyl-3-{3-methyl-1-[5-(trifluoromethyl)-2-pyridyl]-1H-pyrazol-4-ylmethoxy}-1H-pyrazol-5-yl)methanol (4.00 g), activated manganese dioxide (12.18 g) and tetrahydrofuran (100 ml) was stirred overnight at room temperature. The insoluble material was removed by filtration and the filtrate was concentrated. The residue was subjected to silica gel column chromatography, and 1-methyl-3-{3-methyl-1-[5-(trifluoromethyl)-2-pyridyl]-1H-pyrazol-4-ylmethoxy}-1H-pyrazole-5-carbaldehyde (3.39 g, yield 85%) was obtained as colorless crystals from a fraction eluted with ethyl acetate-hexane (1:4, volume ratio). The crystals were recrystallized from ethyl acetate-hexane. melting point: 130-131° C.

### Reference Example 31

A mixture of ethyl 3-propyl-1H-pyrazole-4-carboxylate (25.88 g), 2-chloro-5-(trifluoromethyl)pyridine (25.14 g), potassium carbonate (34.11 g) and N,N-dimethylformamide (300 ml) was stirred overnight at 100° C. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and ethyl 3-propyl-1-[5-(trifluoromethyl)-2-pyridyl]-1H-pyrazole-4-carboxylate (38.45 g, yield 85%) was obtained as colorless crystals from a fraction eluted with ethyl acetate-hexane (1:4, volume ratio). The crystals were recrystallized from isopropyl ether-hexane. melting point: 102-103° C.

¹H-NMR (CDCl₃)δ: 1.03 (3H, t, J=7.2 Hz), 1.38 (3H, t, J=7.0 Hz), 1.66-1.88 (2H, m), 2.86-3.00 (2H, m), 4.33 (2H, q, J=7.0 Hz), 7.99-8.16 (2H, m), 8.65-8.72 (1H, m), 8.99 (1H, s).

### Reference Example 32

To a solution of ethyl 3-propyl-1-[5-(trifluoromethyl)-2-pyridyl]-1H-pyrazole-4-carboxylate (36.41 g) in tetrahydrofuran (300 ml) was dropwise added a 1.0 M solution (250 ml) of diisobutylaluminum hydride in hexane at 0° C., and the mixture was stirred at room temperature for 1 hour. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and {3-propyl-1-[5-(trifluoromethyl)-2-pyridyl]-1H-pyrazol-4-yl}methanol (30.22 g, yield 95%) was obtained as colorless crystals from a fraction eluted with ethyl acetate-hexane (1:1, volume ratio). The crystals were recrystallized from ethyl acetate-hexane. melting point: 120-121° C.

¹H-NMR (CDCl₃)δ: 1.03 (3H, t, J=7.4 Hz), 1.45 (1H, t, J=5.4 Hz), 1.65-1.88 (2H, m), 2.65-2.77 (2H, m), 4.64 (2H, d, J=5.4 Hz), 7.93-8.08 (2H, m), 8.49 (1H, s), 8.61-8.66 (1H, m).

### Reference Example 33

A mixture of {3-propyl-1-[5-(trifluoromethyl)-2-pyridyl]-1H-pyrazol-4-yl}methanol (10.00 g), activated manganese dioxide (29.48 g) and tetrahydrofuran (300 ml) was stirred overnight at room temperature. The insoluble material was removed by filtration and the filtrate was concentrated. The residue was subjected to silica gel column chromatography, and 3-propyl-1-[5-(trifluoromethyl)-2-pyridyl]-1H-pyrazole-4-carbaldehyde (8.87 g, yield 89%) was obtained as colorless crystals from a fraction eluted with ethyl acetate-hexane (1:4, volume ratio). The crystals were recrystallized from ethyl acetate-hexane. melting point: 52-53° C.

¹H-NMR (CDCl₃)δ: 1.03 (3H, t, J=7.2 Hz), 1.68-1.89 (2H, m), 2.88-3.02 (2H, m), 8.07 (1H, dd, J=2.2, 8.8 Hz), 8.14 (1H, d, J=8.8 Hz), 8.67-8.74 (1H, m), 9.04 (1H, s), 10.04 (1H, s).

### Reference Example 34

To a mixture of 3-propyl-1-[5-(trifluoromethyl)-2-pyridyl]-1H-pyrazole-4-carbaldehyde (8.70 g), ethyl diethylphosphonoacetate (8.25 g) and N,N-dimethylformamide (100 ml) was added sodium hydride (60%, in oil, 1.45 g) at 0° C., and the mixture was stirred overnight at room temperature. The reaction mixture was poured into water, and extracted with ethyl acetate. The ethyl acetate layer was washed with dilute hydrochloric acid and then with saturated aqueous sodium chloride solution, dried (MgSO₄) and concentrated. The residue was subjected to silica gel column chromatography, and ethyl (E)-3-{3-propyl-1-[5-(trifluoromethyl)-2-pyridyl]-1H-pyrazol-4-yl}propenoate (10.14 g, yield 93%) was obtained as colorless crystals from a fraction eluted with ethyl acetate-hexane (1:4, volume ratio). The crystals were recrystallized from ethyl acetate-hexane. melting point: 104-105° C.

¹H-NMR (CDCl₃)δ:1.04 (3H, t, J=7.2 Hz), 1.34 (3H, t, J=7.0 Hz) 1.67-1.89 (2H, m), 2.78 (2H, t, J=7.6 Hz), 4.27 (2H, q, J=7.0 Hz), 6.27 (1H, d, J=16.2 Hz), 7.60 (1H, d, J=16.2 Hz), 7.97-8.11 (2H, m), 8.64-8.68 (1H, m), 8.75 (1H, s).

### Reference Example 35

A mixture of ethyl (E)-3-(3-propyl-1-[5-(trifluoromethyl)-2-pyridyl]-1H-pyrazol-4-yl)propenoate (10.00 g), 5% palladium-carbon (3.03 g) and tetrahydrofuran (100 ml) was stirred at room temperature for 1 hour under a hydrogen atmosphere. Palladium-carbon was removed by filtration and the filtrate was concentrated. The residue was subjected to silica gel column chromatography, and ethyl 3-(3-propyl-1-[5-(trifluoromethyl)-2-pyridyl]-1H-pyrazol-4-yl)propionate (9.36 g, yield 93%) was obtained as colorless crystals from a fraction eluted with ethyl acetate-hexane (1:2, volume ratio). The crystals were recrystallized from ethyl acetate-hexane. melting point: 73-74° C.

¹H-NMR (CDCl₃)δ: 1.02 (3H, t, J=7.4 Hz), 1.26 (3H, t, J=7.0 Hz), 1.62-1.86 (2H, m), 2.56-2.68 (4H, m), 2.75-2.86 (2H, m), 4.16 (2H, q, J=7.0 Hz), 7.91-8.04 (2H, m), 8.30 (1H, s), 8.58-8.64 (1H, m).

### Reference Example 36

To a solution of ethyl 3-(3-propyl-1-[5-(trifluoromethyl)-2-pyridyl]-1H-pyrazol-4-yl)propionate (9.10 g) in tetrahydrofuran (100 ml) was dropwise added a 1.0 M solution (60 ml) of diisobutylaluminum hydride in hexane at 0° C., and the mixture was stirred at room temperature for 1 hour. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and 3-{3-propyl-1-[5-(trifluoromethyl)-2-pyridyl]-1H-pyrazol-4-yl}-1-propanol (7.61 g, yield 95%) was obtained as colorless crystals from a fraction eluted with ethyl acetate-hexane (1:1, volume ratio). The crystals were recrystallized from ethyl acetate-hexane. melting point: 96-97° C.

¹H-NMR (CDCl₃)δ: 1.02 (3H, t, J=7.2 Hz), 1.32 (1H, br t), 1.64-1.99 (4H, m), 2.50-2.68 (4H, m), 3.68-3.80 (2H, m), 7.91-8.05 (2H, m), 8.29 (1H, s), 8.58-8.63 (1H, m).

### Reference Example 37

A mixture of ethyl 3-hydroxy-1-methyl-1H-pyrazole-4-carboxylate (25.50 g), benzyl bromide (17.8 ml), potassium carbonate (31.10 g) and N,N-dimethylformamide (250 ml) was stirred overnight at 50° C. The reaction mixture was poured into water, and extracted with ethyl acetate. The ethyl acetate layer was washed with dilute hydrochloric acid and then with saturated aqueous sodium chloride solution, dried (MgSO₄) and concentrated. The residue was subjected to silica gel column chromatography, and ethyl 3-benzyloxy-1-methyl-1H-pyrazole-4-carboxylate (31.90 g, yield 82%) was obtained as colorless crystals from a fraction eluted with ethyl acetate-hexane (1:1, volume ratio). The crystals were recrystallized from ethyl acetate-hexane. melting point: 66-67° C.

### Reference Example 38

To a solution of ethyl 3-benzyloxy-1-methyl-1H-pyrazole-4-carboxylate (18.00 g) in tetrahydrofuran (200 ml) was added lithium aluminum hydride (2.62 g) at 0° C., and the mixture was stirred at room temperature for 1 hour. Sodium sulfate 10 hydrate (22.20 g) was added to the reaction mixture, and the mixture was stirred at room temperature for 1 hour. The precipitate was filtered off and the filtrate was concentrated. The residue was subjected to silica gel column chromatography, and (3-benzyloxy-1-methyl-1H-pyrazol-4-yl)methanol (23.90 g, yield 91%) was obtained as a colorless oil from a fraction eluted with ethyl acetate.

¹H-NMR (CDCl₃)δ: 1.74 (1H, t, J=5.4 Hz), 3.72 (3H, s), 4.47 (2H, d, J=5.4 Hz), 5.24 (2H, s), 7.17 (1H, s), 7.28-7.47 (5H, m).

### Reference Example 39

A mixture of (3-benzyloxy-1-methyl-1H-pyrazol-4-yl)methanol (18.40 g), activated manganese dioxide (40.00 g) and tetrahydrofuran (200 ml) was stirred at room temperature for 9 hours. Manganese dioxide was removed by filtration and the filtrate was concentrated. The residue was subjected to silica gel column chromatography, and 3-benzyloxy-1-methyl-1H-pyrazole-4-carbaldehyde (14.80 g, yield 81%) was obtained as a colorless oil from a fraction eluted with ethyl acetate-hexane (2:1, volume ratio).

¹H-NMR (CDCl₃)δ: 3.78 (3H, s), 5.32 (2H, s), 7.29-7.50 (5H, m), 7.69 (1H, s), 9.76 (1H, s)

### Reference Example 40

To a mixture of potassium t-butoxide (2.24 g) and dimethoxyethane (10 ml) was added a solution of p-toluenesulfonylmethyl isocyanide (2.05 g) in dimethoxyethane (10 ml) at −78° C. and the mixture was stirred for 5 minutes. Then a solution of 3-benzyloxy-1-methyl-1H-pyrazole-4-carbaldehyde (2.16 g) in dimethoxyethane (10 ml) was added. After stirring at the same temperature for 1 hour, the mixture was stirred for 1 hour while raising the temperature to room temperature. To the obtained mixture was added methanol (380 ml), and mixture was refluxed for 1 hour. After cooling, the reaction mixture was poured into saturated aqueous ammonium chloride solution, and extracted with ethyl acetate. The ethyl acetate layer was washed with saturated aqueous sodium chloride solution, dried (MgSO₄) and concentrated. The residue was subjected to silica gel column chromatography, and (3-benzyloxy-1-methyl-1H-pyrazol-4-yl)acetonitrile (1.86 g, yield 82%) was obtained as a colorless oil from a fraction eluted with ethyl acetate-hexane (1:2, volume ratio). ¹H-NMR (CDCl₃)δ: 3.43 (2H, s), 3.74 (3H, s), 5.22 (2H, s), 7.21 (1H, s), 7.29-7.47 (5H, m).

### Reference Example 41

A mixture of (3-benzyloxy-1-methyl-1H-pyrazol-4-yl)acetonitrile (12.0 g), 4N aqueous sodium hydroxide solution (100 ml), tetrahydrofuran (100 ml) and ethanol (100 ml) was refluxed for 21 hours. After cooling, the mixture was neutralized with dilute hydrochloric acid, and extracted with ethyl acetate. The ethyl acetate layer was washed with saturated aqueous sodium chloride solution, dried (MgSO₄) and concentrated. A mixture of the residue, methyl iodide (4.95 ml), potassium carbonate (14.7 g) and N,N-dimethylformamide (100 ml) was stirred overnight at room temperature. The reaction mixture was poured into water, and extracted with ethyl acetate. The ethyl acetate layer was washed with saturated aqueous sodium chloride solution, dried (MgSO₄) and concentrated. The residue was subjected to silica gel column chromatography, and methyl (3-benzyloxy-1-methyl-1H-pyrazol-4-yl)acetate (12.2 g, yield 88%) was obtained as a yellow oily substance from a fraction eluted with ethyl acetate-hexane (1:1, volume ratio).

¹H-NMR (CDCl₃)δ: 3.41 (2H, s), 3.68 (3H, s), 3.73 (3H, s), 5.22 (2H, s), 7.19 (1H, s), 7.30-7.46 (5H, m).

### Reference Example 42

A mixture of methyl (3-benzyloxy-1-methyl-1H-pyrazol-4-yl)acetate (12.2 g), 5% palladium-carbon (25.0 g), tetrahydrofuran (100 ml) and ethanol (100 ml) was stirred under a hydrogen atmosphere for 5 hours. Palladium-carbon was removed by filtration and the filtrate was concentrated to give methyl (3-hydroxy-1-methyl-1H-pyrazol-4-yl)acetate (6.33 g, yield 79%) as colorless crystals. The crystals were recrystallized from tetrahydrofuran-hexane. melting point: 118-119° C.

### Reference Example 43

A mixture of ethyl 3-hydroxy-1-phenyl-1H-pyrazole-4-carboxylate (7.76 g), benzyl bromide (3.97 ml), potassium carbonate (6.91 g) and N,N-dimethylformamide (75 ml) was stirred overnight at 50° C. The reaction mixture was poured into water, and extracted with ethyl acetate. The ethyl acetate layer was washed with dilute hydrochloric acid and then with saturated aqueous sodium chloride solution, dried (MgSO₄) and concentrated. The residue was subjected to silica gel column chromatography, and ethyl 3-benzyloxy-1-phenyl-1H-pyrazole-4-carboxylate (8.29 g, yield 77%) was obtained as colorless crystals from a fraction eluted with ethyl acetate-hexane (1:1, volume ratio). The crystals were recrystallized from ethyl acetate-hexane. melting point: 113-114° C.

### Reference Example 44

To a solution of ethyl 3-benzyloxy-1-phenyl-1H-pyrazole-4-carboxylate (8.06 g) in tetrahydrofuran (100 ml) was added lithium aluminum hydride (0.95 g) at 0° C., and the mixture was stirred at room temperature for 1 hour. To the reaction mixture was added sodium sulfate 10 hydrate (8.06 g), and the mixture was stirred at room temperature for 1 hour. The precipitate was removed by filtration and the filtrate was concentrated. The residue was subjected to silica gel column chromatography, and (3-benzyloxy-1-phenyl-1H-pyrazol-4-yl)methanol (5.91 g, yield 84%) was obtained as colorless crystals from a fraction eluted with ethyl acetate The crystals were recrystallized from ethyl acetate-hexane. melting point: 93-94° C.

### Reference Example 45

A mixture of (3-benzyloxy-1-phenyl-1H-pyrazol-4-yl)methanol (5.61 g), activated manganese dioxide (15.00 g) and tetrahydrofuran (75 ml) was stirred overnight at room temperature. Manganese dioxide was removed by filtration and the filtrate was concentrated. The residue was subjected to silica gel column chromatography, and 3-benzyloxy-1-phenyl-1H-pyrazole-4-carbaldehyde (5.03 g, yield 90%) was obtained as colorless crystals from a fraction eluted with ethyl acetate-hexane (2:1, volume ratio). The crystals were recrystallized from tetrahydrofuran-hexane. melting point: 153-154° C.

### Reference Example 46

To a mixture of potassium t-butoxide (3.82 g) and dimethoxyethane (20 ml) was added a solution of p-toluenesulfonylmethyl isocyanide (3.51 g) in dimethoxyethane (20 ml) at −78° C., and the mixture was stirred for 5 minutes. Then a solution of 3-benzyloxy-1-phenyl-1H-pyrazole-4-carbaldehyde (4.73 g) in dimethoxyethane (80 ml) was added. After stirring at the same temperature for 1 hour, the mixture was stirred for 1 hour while raising the temperature to room temperature. Methanol (100 ml) was added to the obtained mixture, and the mixture was refluxed for 1 hour. After cooling, the reaction mixture was poured into saturated aqueous ammonium chloride solution, and extracted with ethyl acetate. The ethyl acetate layer was washed with saturated aqueous sodium chloride solution, dried (MgSO₄) and concentrated. The residue was subjected to silica gel column chromatography, and (3-benzyloxy-1-phenyl-1H-pyrazol-4-yl)acetonitrile (3.31 g, yield 67%) was obtained as colorless crystals from a fraction eluted with ethyl acetate-hexane (1:3, volume ratio). The crystals were recrystallized from tetrahydrofuran-hexane. melting point: 102-103° C.

### Reference Example 47

A mixture of (3-benzyloxy-1-phenyl-1H-pyrazol-4-yl)acetonitrile (3.01 g), 6N aqueous sodium hydroxide solution (25 ml), tetrahydrofuran (25 ml) and ethanol (25 ml) was refluxed for 3 days. After cooling, the mixture was neutralized with dilute hydrochloric acid, and extracted with ethyl acetate. The ethyl acetate layer was washed with saturated aqueous sodium chloride solution, dried (MgSO₄) and concentrated to give (3-benzyloxy-1-phenyl-1H-pyrazol-4-yl)acetic acid (2.63 g, yield 82%) as colorless crystals. The crystals were recrystallized from acetone-hexane. melting point: 105-106° C.

### Reference Example 48

A mixture of (3-benzyloxy-1-phenyl-1H-pyrazol-4-yl)acetic acid (2.47 g), methyl iodide (0.75 ml), potassium carbonate (2.21 g) and N,N-dimethylformamide (25 ml) was stirred at room temperature for 1 hour. The reaction mixture was poured into water, and extracted with ethyl acetate. The ethyl acetate layer was washed with saturated aqueous sodium chloride solution, dried (MgSO₄) and concentrated. The residue was subjected to silica gel column chromatography, and methyl (3-benzyloxy-1-phenyl-1H-pyrazol-4-yl)acetate (2.55 g, yield 99%) was obtained as colorless crystals from a fraction eluted with ethyl acetate-hexane (1:3, volume ratio). The crystals were recrystallized from ethyl acetate-hexane. melting point: 74-75° C.

### Reference Example 49

A mixture of methyl (3-benzyloxy-1-phenyl-1H-pyrazol-4-yl)acetate (2.35 g), 5% palladium-carbon (4.00 g), tetrahydrofuran (25 ml) and methanol (25 ml) was stirred for 1 hour under a hydrogen atmosphere. Palladium-carbon was removed by filtration and the filtrate was concentrated to give methyl (3-hydroxy-1-phenyl-1H-pyrazol-4-yl)acetate (1.58 g, yield 93%) as colorless crystals. The crystals were recrystallized from ethyl acetate-hexane. melting point: 144-145° C.

### Reference Example 50

A mixture of [2-(1,3-dioxolan-2-yl)ethyl]triphenylphosphonium bromide (18.86 g), sodium hydride (60%, in oil, 1.70 g) and N,N-dimethylformamide (100 ml) was stirred at room temperature for 30 minutes. 3-Propyl-1-[5-(trifluoromethyl)-2-pyridyl]-1H-pyrazole-4-carbaldehyde (9.00 g) was added thereto and the mixture was stirred at 70° C. for 5 hours. The reaction mixture was poured into water, and extracted with ethyl acetate. The ethyl acetate layer was washed with saturated aqueous sodium chloride solution, dried (MgSO₄) and concentrated. A mixture of the residue, 5% palladium-carbon (2.04 g) and tetrahydrofuran (100 ml) was stirred for 1 hour under a hydrogen atmosphere. Palladium-carbon was removed by filtration and the filtrate was concentrated. The obtained residue was dissolved in tetrahydrofuran (150 ml), and 1N hydrochloric acid (200 ml) and methanol (50 ml) were added, which was followed by stirring at room temperature for 2 hours. The reaction mixture was poured into water, and extracted with ethyl acetate. The ethyl acetate layer was washed with saturated aqueous sodium chloride solution, dried (MgSO₄) and concentrated. The residue was subjected to silica gel column chromatography and 4-{3-propyl-1-[5-(trifluoromethyl)-2-pyridyl]-1H-pyrazol-4-yl}butanal (8.08 g, yield 78%) was obtained as colorless crystals from a fraction eluted with ethyl acetate-hexane (1:4, volume ratio). The crystals were recrystallized from ethyl acetate-hexane. melting point: 71-72° C.

### Reference Example 51

To a mixture of 4-{3-propyl-1-[5-(trifluoromethyl)-2-pyridyl]-1H-pyrazol-4-yl}butanal (7.85 g), methanol (20 ml) and tetrahydrofuran (20 ml) was slowly added sodium borohydride (700 mg) at 0° C., and the mixture was stirred at room temperature for 30 minutes. The reaction mixture was poured into dilute hydrochloric acid, and extracted with ethyl acetate. The ethyl acetate layer was washed with saturated aqueous sodium chloride solution, dried (MgSO₄) and concentrated to give 4-{3-propyl-1-[5-(trifluoromethyl)-2-pyridyl]-1H-pyrazol-4-yl}-1-butanol (7.48 g, yield 95%) as colorless crystals. The crystals were recrystallized from ethyl acetate-hexane. melting point: 80-81° C.

### Reference Example 52

To a mixture of 2-(1,3-dioxolan-2-yl)ethyltetraphenylphosphonium bromide (18.95 g) and N,N-dimethylformamide (178 ml) was added sodium hydride (60%, in oil, 1.71 g) at 0° C. and the mixture was stirred at room temperature for 30 minutes. Then, 3-isopropyl-1-[5-(trifluoromethyl)-2-pyridinyl]-1H-pyrazole-4-carbaldehyde (10.09 g) was added and the mixture was stirred at room temperature overnight, and at 70° C. for 4 hours. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and a colorless oil was obtained from a fraction eluted with ethyl acetate-hexane (1:15, volume ratio). A mixture of the obtained oily substance, 5% palladium-carbon (1.28 g) and ethanol (174 ml) was stirred at room temperature for 3.5 hours under a hydrogen atmosphere. Palladium-carbon was removed by filtration and the filtrate was concentrated to give 2-{4-[3-(1,3-dioxolan-2-yl)propyl]-3-isopropyl-1H-pyrazol-1-yl}-5-(trifluoromethyl)pyridine (12.84 g, yield 98%) as a colorless oil.

¹H-NMR (CDCl₃)δ: 1.32 (6H, d, J=7.0 Hz), 1.72-1.82 (4H, m) 2.46-2.58 (2H, m), 2.92-3.10 (1H, m), 3.82-4.00 (4H, m), 4.88-4.96 (1H, m), 7.88-7.98 (1H, m), 8.02 (1H, d, J=8.4 Hz), 8.27 (1H, s), 8.56-8.61 (1H, m).

### Reference Example 53

A mixture of 2-{4-[3-(1,3-dioxolan-2-yl)propyl]-3-isopropyl-1H-pyrazol-1-yl}-5-(trifluoromethyl)pyridine (12.84 g), 1N hydrochloric acid (100 ml), tetrahydrofuran (100 ml) and methanol (100 ml) was stirred overnight at 50° C. The reaction mixture was concentrated under reduced pressure, and extracted with ethyl acetate. The ethyl acetate layer was washed with saturated aqueous sodium chloride solution, dried (MgSO₄) and concentrated. The residue was subjected to silica gel column chromatography, and 4-{3-isopropyl-1-[5-(trifluoromethyl)-2-pyridinyl]-1H-pyrazol-4-yl}butyraldehyde (11.25 g, yield 99%) was obtained as a colorless oil from a fraction eluted with ethyl acetate-hexane (1:2, volume ratio).

¹H-NMR (CDCl₃)δ: 1.32 (6H, d, J=6.9 Hz), 1.90-2.06 (2H, m), 2.44-2.60 (4H, m), 2.94-3.07 (1H, m), 7.90-7.98 (1H, m), 8.02 (1H, d, J=8.7 Hz), 8.27 (1H, s), 8.55-8.61 (1H, m), 9.78-9.81 (1H, m).

### Reference Example 54

To a solution of 4-{3-isopropyl-1-[5-(trifluoromethyl)-2-pyridinyl]-1H-pyrazol-4-yl}butyraldehyde (11.25 g) in ethanol (170 ml) was added sodium borohydride (1.57 g) at room temperature and the mixture was stirred for 1 hour. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and 4-{3-isopropyl-1-[5-(trifluoromethyl)-2-pyridinyl]-1H-pyrazol-4-yl}-1-butanol (6.11 g, yield 54%) was obtained as colorless crystals from a fraction eluted with ethyl acetate-hexane (1:4, volume ratio). Along therewith, 4-{3-isopropyl-1-[5-(trifluoromethyl)-2-pyridinyl]-1H-pyrazol-4-yl}butyraldehyde (2.46 g), which was a starting material, was also recovered. The obtained colorless crystals were recrystallized from ethyl acetate-hexane. melting point: 67-68° C.

### Reference Example 55

A mixture of ethyl (3-ethoxy-1H-pyrazol-4-yl)acetate (18.95 g), sodium hydride (60%, in oil, 4.59 g) and N,N-dimethylformamide (478 ml) was stirred at room temperature for 1 hour, to which 2-chloro-5-(trifluoromethyl)pyridine (20.82 g) was added and the mixture was stirred overnight. The reaction mixture was poured into water, and extracted with ethyl acetate. The ethyl acetate layer was washed with saturated aqueous sodium chloride solution, dried (MgSO₄) and concentrated. The residue was subjected to silica gel column chromatography, and ethyl {3-ethoxy-1-[5-(trifluoromethyl)-2-pyridinyl]-1H-pyrazol-4-yl}acetate (11.27 g, yield 41%) was obtained as a colorless oil from a fraction eluted with ethyl acetate-hexane (1:9, volume ratio).

¹H-NMR (CDCl₃)δ: 1.29 (3H, t, J=7.4 Hz), 1.42 (3H, t, J=7.0 Hz), 3.46 (2H, s), 4.20 (2H, q, J=7.4 Hz), 4.36 (2H, q, J=7.0 Hz), 7.83 (1H, d, J=8.8 Hz), 7.84-7.96 (1H, m), 8.39 (1H, s), 8.54-8.60 (1H, m).

### Reference Example 56

To a solution of ethyl {3-ethoxy-1-[5-(trifluoromethyl)-2-pyridinyl]-1H-pyrazol-4-yl}acetate (11.27 g) in tetrahydrofuran (400 ml) was dropwise added a 1.0 M solution (117 ml) of diisobutylaluminum hydride in hexane at 0° C., and the mixture was stirred at room temperature for 3 hours. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and 2-{3-ethoxy-1-[5-(trifluoromethyl)-2-pyridinyl]-1H-pyrazol-4-yl}ethanol (4.38 g, yield 45%) was obtained as pale-yellow crystals from a fraction eluted with ethyl acetate-hexane (1:4, volume ratio). The crystals were recrystallized from ethyl acetate-hexane. melting point: 75-76° C.

### Reference Example 57

To a solution of ethyl 3-(3-hydroxy-1H-pyrazol-4-yl)propanoate (7.40 g) in tetrahydrofuran (100 ml) were added di-tert-butyl dicarbonate (9.71 ml) and triethylamine (5.89 ml) at room temperature and the mixture was stirred overnight. The reaction mixture was concentrated to give a residue. To a mixture of the obtained residue, benzyl alcohol (5.00 ml), tributylphosphine (20.1 ml) and tetrahydrofuran (805 ml) was added a 40% toluene solution (52.9 ml) of 1,1′-diethyl azodicarboxylate at room temperature and the mixture was stirred overnight. The reaction solution was concentrated. The residue was subjected to silica gel column chromatography, and tert-butyl 3-benzyloxy-4-(2-ethoxycarbonylethyl)-1H-pyrazole-1-carboxylate (5.08 g, yield 34%) was obtained as a colorless oil from a fraction eluted with ethyl acetate-hexane (1:6, volume ratio).

¹H-NMR (CDCl₃)δ: 1.23 (3H, t, J=6.9 Hz), 1.61 (9H, s), 2.53-2.60 (2H, m), 2.66-2.73 (2H, m), 4.11 (2H, q, J=6.9 Hz), 5.34 (2H, s), 7.27-7.46 (5H, m), 7.65 (1H, s).

### Reference Example 58

To a solution of tert-butyl 3-benzyloxy-4-(2-ethoxycarbonylethyl)-1H-pyrazole-1-carboxylate (5.08 g) in ethyl acetate (13.6 ml) was added 4N ethyl acetate solution (43.6 ml) of hydrochloric acid and the mixture was stirred overnight. The reaction mixture was poured into saturated aqueous sodium hydrogen carbonate solution and extracted with ethyl acetate. The ethyl acetate layer was washed with saturated aqueous sodium chloride solution, dried (MgSO₄) and concentrated to give ethyl 3-(3-benzyloxy-1H-pyrazol-4-yl)propanoate (3.92 g, quantitative) as a colorless oil.

¹H-NMR (CDCl₃)δ: 1.22 (3H, t, J=7.2 Hz), 2.04-2.59 (2H, m), 2.69-2.75 (2H, m), 4.10 (2H, q, J=7.2 Hz), 5.25 (2H, s), 7.19 (1H, s), 7.25-7.45 (5H, m).

### Reference Example 59

A mixture of ethyl 3-(3-benzyloxy-1H-pyrazol-4-yl)propanoate (2.84 g), sodium hydride (60%, in oil, 497 mg) and N,N-dimethylformamide (104 ml) was stirred at room temperature for 1 hour and 2-chloro-5-(trifluoromethyl)pyridine (2.26 g) was added. The mixture was stirred overnight. The reaction mixture was poured into water, and extracted with ethyl acetate. The ethyl acetate layer was washed with saturated aqueous sodium chloride solution, dried (MgSO₄) and concentrated. The residue was subjected to silica gel column chromatography, and ethyl 3-{3-benzyloxy-1-[5-(trifluoromethyl)-2-pyridinyl]-1H-pyrazol-4-yl}propanoate (3.14 g, yield 72%) was obtained as a colorless oil from a fraction eluted with ethyl acetate-hexane (1:4, volume ratio).

¹H-NMR (CDCl₃)δ: 1.24 (3H, t, J=7.2 Hz), 2.57-2.65 (2H, m), 2.74-2.81 (2H, m), 4.12 (2H, q, J=7.2 Hz), 5.35 (2H, s), 7.39-7.43 (3H, m), 7.44-7.50 (2H, m), 7.82 (1H, d, J=8.4 Hz), 7.89-7.94 (1H, m), 8.22 (1H, s), 8.53-8.57 (1H, m).

### Reference Example 60

To a solution of ethyl 3-{3-benzyloxy-1-[5-(trifluoromethyl)-2-pyridinyl]-1H-pyrazol-4-yl}propanoate (3.14 g) in tetrahydrofuran (75 ml) was dropwise added a 1.0 M solution (16.5 ml) of diisobutylaluminum hydride in hexane at 0° C., and the mixture was stirred at room temperature for 3 hours. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and 3-{3-benzyloxy-1-[5-(trifluoromethyl)-2-pyridinyl]-1H-pyrazol-4-yl}-1-propanol (2.41 g, yield 85%) was obtained as colorless crystals from a fraction eluted with ethyl acetate-hexane (1:4, volume ratio). The crystals were recrystallized from ethyl acetate-hexane. melting point: 79-81° C.

### Reference Example 61

To a mixture of 4-{3-isopropyl-1-[5-(trifluoromethyl)-2-pyridinyl]-1H-pyrazol-4-yl}-1-butanol (1.20 g), triethylamine (613 μL) and tetrahydrofuran (37 ml) was added methanesulfonyl chloride (341 μL) at room temperature, and the mixture was stirred overnight. The reaction solution was concentrated. The residue was subjected to silica gel column chromatography, and 4-{3-isopropyl-1-[5-(trifluoromethyl)-2-pyridinyl]-1H-pyrazol-4-yl}butyl methanesulfonate (1.25 g, yield 84%) was obtained as colorless crystals from a fraction eluted with ethyl acetate-hexane (1:4, volume ratio). The crystals were recrystallized from ethyl acetate-hexane. melting point: 87-89° C.

### Reference Example 62

To a mixture of 5-benzyloxy-2-methoxybenzaldehyde (3.45 g), ethyl diethylphosphonoacetate (3.41 ml) and N,N-dimethylformamide (100 ml) was added sodium hydride (60%, in oil, 684 mg) at 0° C. and the mixture was stirred at room temperature for 2 days. The reaction mixture was poured into 0.1N hydrochloric acid, and extracted with ethyl acetate. The ethyl acetate layer was washed with saturated aqueous sodium chloride solution, dried (MgSO₄) and concentrated. The residue was subjected to silica gel column chromatography, and a pale-yellow oily substance was obtained from a fraction eluted with ethyl acetate-hexane (1:9, volume ratio). A mixture of the obtained oily substance, 5% palladium-carbon (1.00 g) and ethanol (150 ml) was stirred at room temperature for 2 hours under a hydrogen atmosphere. Palladium-carbon was removed by filtration and the filtrate was concentrated. The residue was subjected to silica gel column chromatography, and ethyl 3-(5-hydroxy-2-methoxyphenyl)propanoate (2.54 g, yield 80%) was obtained as a brown oily substance from a fraction eluted with ethyl acetate-hexane (1:6, volume ratio).

¹H-NMR (CDCl₃)δ: 1.24 (3H, t, J=6.8 Hz), 2.52-2.64 (2H, m), 2.82-2.94 (2H, m), 3.77 (3H, s), 4.12 (2H, q, J=6.8 Hz), 4.94 (1H, brs), 6.61-6.74 (3H, m).

### Reference Example 63

To a mixture of ethyl 3-(3-phenyl-1H-pyrazol-4-yl)propionate (3.00 g), 2-chloro-5-(trifluoromethyl)pyridine (2.35 g) and N,N-dimethylformamide (30 ml) was added sodium hydride (60%, in oil, 620 mg) at 0° C., and the mixture was stirred overnight at room temperature. The reaction mixture was poured into water, and extracted with ethyl acetate. The ethyl acetate layer was washed with dilute hydrochloric acid and then with saturated aqueous sodium chloride solution, dried (MgSO₄) and concentrated. The residue was subjected to silica gel column chromatography, and a colorless oil was obtained from a fraction eluted with ethyl acetate-hexane (1:4, volume ratio). To a solution of the obtained colorless oil in tetrahydrofuran (50 ml) was dropwise added a 1.0 M solution (30 ml) of diisobutylaluminum hydride in hexane at 0° C., and the mixture was stirred at room temperature for 1 hour. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and 3-{3-phenyl-1-[5-(trifluoromethyl)-2-pyridyl]-1H-pyrazol-4-yl}-1-propanol (3.85 g, yield 86%) was obtained as colorless crystals from a fraction eluted with ethyl acetate-hexane (1:1, volume ratio). The crystals were recrystallized from ethyl acetate-hexane. melting point: 99-100° C.

### Reference Example 64

A mixture of {3-methyl-1-[5-(trifluoromethyl)-2-pyridyl]-1H-pyrazol-4-yl}methanol (10.05 g), activated manganese dioxide (31.48 g) and tetrahydrofuran (200 ml) was stirred overnight at room temperature. The insoluble material was removed by filtration and the filtrate was concentrated. The residue was subjected to silica gel column chromatography, and 3-methyl-1-[5-(trifluoromethyl)-2-pyridyl]-1H-pyrazole-4-carbaldehyde (8.94 g, yield 90%) was obtained as colorless crystals from a fraction eluted with ethyl acetate-hexane (1:4, volume ratio). The crystals were recrystallized from ethyl acetate-hexane. melting point: 226-227° C.

### Reference Example 65

To a mixture of 3-methyl-1-[5-(trifluoromethyl)-2-pyridyl]-1H-pyrazole-4-carbaldehyde (8.30 g), ethyl diethylphosphonoacetate (8.50 g) and N,N-dimethylformamide (75 ml) was added sodium hydride (60%, in oil, 1.50 g) at 0° C. and the mixture was stirred overnight at room temperature. The reaction mixture was poured into water, and extracted with ethyl acetate. The ethyl acetate layer was washed with dilute hydrochloric acid and then with saturated aqueous sodium chloride solution, dried (MgSO₄) and concentrated. The residue was subjected to silica gel column chromatography, and ethyl (E)-3-(3-methyl-1-[5-(trifluoromethyl)-2-pyridyl]-1H-pyrazol-4-yl)propenoate (9.53 g, yield 90%) was obtained as colorless crystals from a fraction eluted with ethyl acetate-hexane (1:4, volume ratio). The crystals were recrystallized from ethyl acetate-hexane. melting point: 131-132° C.

### Reference Example 66

A mixture of ethyl (E)-3-(3-methyl-1-[5-(trifluoromethyl)-2-pyridyl]-1H-pyrazol-4-yl)propenoate (9.00 g), 5% palladium-carbon (2.42 g) and tetrahydrofuran (100 ml) was stirred at room temperature for 1 hour under a hydrogen atmosphere. Palladium-carbon was removed by filtration and the filtrate was concentrated. The residue was subjected to silica gel column chromatography, and ethyl 3-(3-methyl-1-[5-(trifluoromethyl)-2-pyridyl]-1H-pyrazol-4-yl)propionate (8.45 g, yield 93%) was obtained as colorless crystals from a fraction eluted with ethyl acetate-hexane (1:2, volume ratio). The crystals were recrystallized from ethyl acetate-hexane. melting point: 50-51° C.

### Reference Example 67

To a solution of ethyl 3-(3-methyl-1-[5-(trifluoromethyl)-2-pyridyl]-1H-pyrazol-4-yl)propionate (7.00 g) in tetrahydrofuran (100 ml) was dropwise added a 1.0 M solution (50 ml) of diisobutylaluminum hydride in hexane at 0° C., and the mixture was stirred at room temperature for 1 hour. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and 3-{3-methyl-1-[5-(trifluoromethyl)-2-pyridyl]-1H-pyrazol-4-yl}-1-propanol (5.63 g, yield 92%) was obtained as colorless crystals from a fraction eluted with ethyl acetate-hexane (1:1, volume ratio). The crystals were recrystallized from ethyl acetate-hexane. melting point: 103-104° C.

### Reference Example 68

To a solution of 2-benzyloxy-3-methoxybenzaldehyde (9.90 g) in tetrahydrofuran (100 ml) was added lithium aluminum hydride (1.15 g) at 0° C., and the mixture was stirred at room temperature for 1 hour. Sodium sulfate 10 hydrate (11.03 g) was added to the reaction mixture, and the mixture was stirred at room temperature for 1 hour. The precipitate was removed by filtration and the filtrate was concentrated. The residue was subjected to silica gel column chromatography, and 2-benzyloxy-3-methoxybenzyl alcohol (9.94 g, quantitative) was obtained as a colorless oil from a fraction eluted with ethyl acetate.

¹H-NMR (CDCl₃)δ: 1.97 (1H, t, J=6.6 Hz), 3.91 (3H, s), 4.55 (2H, d, J=6.6 Hz), 5.09 (2H, s), 6.86-6.96 (2H, m), 7.01-7.12 (1H, m), 7.28-7.49 (5H, m).

### Reference Example 69

To a mixture of 2-benzyloxy-3-methoxybenzyl alcohol (9.90 g), acetone cyanohydrin (4.60 g), triphenylphosphine (16.21 g) and tetrahydrofuran (200 ml) was dropwise added a 40% toluene solution (26.49 g) of diethyl azodicarboxylate at room temperature, and the mixture was stirred overnight. The reaction solution was concentrated. The residue was subjected to silica gel column chromatography, and (2-benzyloxy-3-methoxyphenyl)acetonitrile (8.62 g, yield 84%) was obtained as a colorless oil from a fraction eluted with ethyl acetate-hexane (1:4, volume ratio).

¹H-NMR (CDCl₃)δ: 3.53 (2H, s), 3.92 (3H, s), 5.09 (2H, s), 6.90-7.14 (3H, m), 7.32-7.46 (5H, m).

### Reference Example 70

A mixture of (2-benzyloxy-3-methoxyphenyl)acetonitrile (8.62 g), 8N aqueous sodium hydroxide solution (40 ml) and ethanol (200 ml) was stirred under reflux overnight. After cooling, the reaction mixture was acidified by slowly adding conc. hydrochloric acid (30 ml). After concentration, the residue was dissolved in ethyl acetate. The obtained ethyl acetate solution was washed with saturated aqueous sodium chloride solution, dried (MgSO₄) and concentrated. A mixture of the residue, a 10% solution (200 ml) of hydrochloric acid in methanol and methanol (200 ml) was stirred overnight at room temperature. After concentration, the residue was dissolved in ethyl acetate. The obtained ethyl acetate solution was washed with saturated aqueous sodium chloride solution, dried (MgSO₄) and concentrated. The residue was subjected to silica gel column chromatography, and methyl (2-benzyloxy-3-methoxyphenyl)acetate (7.40 g, yield 76%) was obtained as a colorless oil from a fraction eluted with ethyl acetate-hexane (1:4, volume ratio).

¹H-NMR (CDCl₃)δ: 3.61 (5H, s), 3.89 (3H, s), 5.03 (2H, s), 6.79-7.10 (3H, m), 7.25-7.56 (5H, m).

### Reference Example 71

A mixture of methyl (2-benzyloxy-3-methoxyphenyl)acetate (7.40 g), 5% palladium-carbon (1.39 g) and tetrahydrofuran (100 ml) was stirred overnight at room temperature under a hydrogen atmosphere. Palladium-carbon was removed by filtration and the filtrate was concentrated. The residue was subjected to silica gel column chromatography, and was obtained from a fraction eluted with ethyl acetate-hexane (1:4, volume ratio) to give methyl (2-hydroxy-3-methoxyphenyl)acetate (5.01 g, yield 99%) as a colorless oil.

¹H-NMR (CDCl₃)δ: 3.68 (2H, s), 3.70 (3H, s), 3.88 (3H, s), 5.88 (1H, s), 6.76-6.86 (3H, m).

### Reference Example 72

A mixture of methyl 3,5-dihydroxybenzoate (500 mg), benzyl bromide (17.7 ml), potassium carbonate (20.62 g) and N,N-dimethylformamide (250 ml) was stirred overnight at room temperature. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and colorless crystals were obtained from a fraction eluted with ethyl acetate-hexane (1:3, volume ratio). A mixture of the obtained colorless crystals, methyl iodide (4.6 ml), potassium carbonate (7.90 g) and N,N-dimethylformamide (150 ml) was stirred overnight at room temperature. The reaction mixture was poured into water, and extracted with diethyl ether. The diethyl ether layer was washed with saturated aqueous sodium chloride solution, dried (MgSO₄) and concentrated to give methyl 3-benzyloxy-5-methoxybenzoate (15.54 g, yield 38%) as a pale-yellow oily substance.

¹H-NMR (CDCl₃)δ: 3.82 (3H, s), 3.91 (3H, s), 5.08 (2H, s), 6.73 (1H, t, J=2.3 Hz), 7.19-7.46 (7H, m).

### Reference Example 73

To a mixture of lithium aluminum hydride (5.40 g) and tetrahydrofuran (100 ml) was slowly added a solution of methyl 3-benzyloxy-5-methoxybenzoate (15.54 g) in tetrahydrofuran (20 ml) at 0° C., and the mixture was stirred at room temperature for 30 minutes. Acetone (80 ml) was slowly added to decompose excess lithium aluminum hydride, and brine (15.4 ml) was added. The precipitate was removed by filtration and the filtrate was concentrated. The residue was subjected to silica gel column chromatography, and (3-benzyloxy-5-methoxyphenyl)methanol (14.00 g, quantitative) was obtained as a colorless oil from a fraction eluted with ethyl acetate-hexane (2:3, volume ratio).

¹H-NMR (CDCl₃)δ: 1.69 (1H, t, J=6.1 Hz), 3.79 (3H, s), 4.63 (2H, d, J=6.1 Hz), 5.05 (2H, s), 6.47 (1H, t, J=2.3 Hz), 6.53-6.55 (1H, m), 6.66-6.68 (1H, m), 7.29-7.45 (5H, m).

### Reference Example 74

A mixture of (3-benzyloxy-5-methoxyphenyl)methanol (6.03 g), activated manganese dioxide (18.0 g) and tetrahydrofuran (80 ml) was stirred overnight at room temperature. The insoluble material was removed by filtration and the filtrate was concentrated. The residue was subjected to silica gel column chromatography, and a colorless oil was obtained from a fraction eluted with ethyl acetate-hexane (1:3, volume ratio). To a mixture of the obtained oil, ethyl diethylphosphonoacetate (4.84 g) and N,N-dimethylformamide (50 ml) was added sodium hydride (60%, in oil, 950 mg) at 0° C., and the mixture was stirred overnight at room temperature. The reaction mixture was poured into water, and extracted with ethyl acetate. The ethyl acetate layer was washed with saturated aqueous sodium chloride solution, dried (MgSO₄) and concentrated. The residue was subjected to silica gel column chromatography, and ethyl (E)-3-(3-benzyloxy-5-methoxyphenyl)propenoate (3.96 g, yield 51%) was obtained as a pale-yellow oily substance from a fraction eluted with ethyl acetate-hexane (1:3, volume ratio).

¹H-NMR (CDCl₃)δ: 1.34 (3H, t, J=7.1 Hz), 3.80 (3H, s), 4.26 (2H, q, J=7.1 Hz), 5.06 (2H, s), 6.39 (1H, d, J=15.9 Hz), 6.57 (1H, t, J=2.2 Hz), 6.68 (1H, t, J=1.7 Hz), 6.75 (1H, t, J=1.7 Hz), 7.30-7.45 (5H, m), 7.59 (1H, d, J=15.9 Hz).

### Reference Example 75

A mixture of ethyl (E)-3-(3-benzyloxy-5-methoxyphenyl)propenoate (3.96 g), 5% palladium-carbon (0.4 g) and ethanol (25 ml) was stirred at room temperature overnight under a hydrogen atmosphere. Palladium-carbon was removed by filtration and the filtrate was concentrated. The residue was subjected to silica gel column chromatography, and ethyl 3-(3-hydroxy-5-methoxyphenyl)propionate (2.78 g, yield 98%) was obtained as a pale-yellow oily substance from a fraction eluted with ethyl acetate-hexane (2:3, volume ratio).

¹H-NMR (CDCl₃)δ: 1.25 (3H, t, J=7.1 Hz), 2.60 (2H, t, J=7.8 Hz), 2.86 (2H, t, J=7.8 Hz), 3.76 (3H, s), 4.14 (2H, q, J=7.1 Hz), 5.22 (1H, s), 6.25-6.35 (3H, m).

### Reference Example 76

To a mixture of (3-benzyloxy-5-methoxyphenyl)methanol (8.00 g), acetone cyanohydrin (4.65 ml), tributylphosphine (13.3 g) and tetrahydrofuran (200 ml) was added 1,1′-azodicarbonyldipiperidine (16.53 g) at room temperature and the mixture was stirred overnight. The reaction solution was concentrated. The residue was subjected to silica gel column chromatography, and (3-benzyloxy-5-methoxyphenyl)acetonitrile (5.77 g, yield 70%) was obtained as a yellow oily substance from a fraction eluted with ethyl acetate-hexane (1:2, volume ratio).

¹H-NMR (CDCl₃)δ: 3.68 (2H, s), 3.78 (3H, s), 5.05 (2H, s) 6.46-6.56 (3H, m), 7.30-7.45 (5H, m).

### Reference Example 77

A mixture of (3-benzyloxy-5-methoxyphenyl)acetonitrile (5.77 g), potassium hydroxide (4.50 g) and ethylene glycol (50 ml) was stirred overnight 120° C. The reaction mixture was poured into water, and washed with diethyl ether. The aqueous layer was acidified by adding hydrochloric acid, and extracted with ethyl acetate. The ethyl acetate layer was washed with saturated aqueous sodium chloride solution, dried (MgSO₄) and concentrated to give a residue. A mixture of the obtained residue, methyl iodide (1.80 ml), potassium carbonate (4.00 g) and N,N-dimethylformamide (50 ml), and the mixture was stirred at room temperature for 1 hour. The reaction mixture was poured into water, and extracted with ethyl acetate. The ethyl acetate layer was washed with saturated aqueous sodium chloride solution, dried (MgSO₄) and concentrated. The residue was subjected to silica gel column chromatography, and methyl (3-benzyloxy-5-methoxyphenyl)acetate (4.43 g, yield 68%) was obtained as a colorless oil from a fraction eluted with ethyl acetate-hexane (1:4, volume ratio).

¹H-NMR (CDCl₃)δ: 3.56 (2H, s), 3.69 (3H, s), 3.77 (3H, s), 5.03 (2H, s), 6.44-6.47 (2H, m), 6.51-6.54 (1H, m), 7.29-7.45 (5H, m).

### Reference Example 78

A mixture of methyl (3-benzyloxy-5-methoxyphenyl)acetate (4.43 g), 5% palladium-carbon (0.44 g) and ethanol (25 ml) was stirred overnight at room temperature under a hydrogen atmosphere. Palladium-carbon was removed by filtration and the filtrate was concentrated. The residue was subjected to silica gel column chromatography, and methyl (3-hydroxy-5-methoxyphenyl)acetate (2.97 g, yield 97%) was obtained as a colorless oil from a fraction eluted with ethyl acetate-hexane (2:3, volume ratio).

¹H-NMR (CDCl₃)δ: 3.54 (2H, s), 3.70 (3H, s), 3.76 (3H, s), 5.38 (1H, br s), 6.32 (1H, t, J=2.3 Hz), 6.35-6.42 (2H, m).

### Reference Example 79

A mixture of (4-hydroxyphenyl)acetonitrile (15.0 g), benzyl bromide (13.6 ml), potassium carbonate (15.6 g) and N,N-dimethylformamide (100 ml) was stirred overnight at room temperature. The reaction mixture was poured into water, The precipitated crystals were collected by filtration, washed well with water and dried to give (4-benzyloxyphenyl)acetonitrile (24.12 g, yield 96%). melting point: 70-71° C.

¹H-NMR (CDCl₃)δ: 3.68 (2H, s), 5.07 (2H, s), 6.95-6.99 (2H, m), 7.21-7.25 (2H, m), 7.30-7.45 (5H, m).

### Reference Example 80

To a mixture of (4-benzyloxyphenyl)acetonitrile (600 mg), methyl iodide (20.0 ml) and dimethyl sulfoxide (200 ml) was slowly added 50% aqueous sodium hydroxide solution at 0° C., and the mixture was stirred at room temperature for 3 hours. The reaction mixture was poured into water, and the precipitated crystals were collected by filtration, washed well with water and dried to give 2-(4-benzyloxyphenyl)-2-methylpropanenitrile (25.88 g, yield 99%). melting point: 63-64° C.

¹H-NMR (CDCl₃)δ: 1.70 (6H, s), 5.07 (2H, s), 6.95-7.00 (2H, m), 7.30-7.45 (7H, m).

### Reference Example 81

A mixture of 2-(4-benzyloxyphenyl)-2-methylpropanenitrile (25.88 g), potassium hydroxide (20.34 g) and ethylene glycol (200 ml) was stirred at 120° C. for 2 days. The reaction mixture was poured into ice water, acidified by adding hydrochloric acid, and extracted with ethyl acetate. The ethyl acetate layer was washed with saturated aqueous sodium chloride solution, dried (MgSO₄) and concentrated. The obtained colorless crystals were collected by filtration to give 2-(4-benzyloxyphenyl)-2-methylpropanoic acid (27.62 g, yield 99%). melting point: 128-130° C.

¹H-NMR (CDCl₃)δ: 1.58 (6H, s), 5.05 (2H, s), 6.92-6.97 (2H, m), 7.29-7.45 (7H, m).

### Reference Example 82

A mixture of 2-(4-benzyloxyphenyl)-2-methylpropanoic acid (27.62 g), sulfuric acid (6 ml) and ethanol (500 ml) was refluxed for 14 hours. The reaction mixture was poured into ice water, and the precipitated crystals were collected by filtration, washed well with aqueous sodium hydrogen carbonate and water and dried to give ethyl 2-(4-benzyloxyphenyl)-2-methylpropanoate (2820 g, yield 92%). melting point: 54-55° C.

¹H-NMR (CDCl₃)δ: 1.82 (3H, t, J=7.1 Hz), 1.55 (6H, s), 4.11 (2H, q, J=7.1 Hz), 5.04 (2H, s), 6.90-6.95 (2H, m), 7.24-7.45 (7H, m).

### Reference Example 83

A mixture of ethyl 2-(4-benzyloxyphenyl)-2-methylpropanoate (28.20 g), 5% palladium-carbon (2.8 g) and ethanol (100 ml) was stirred overnight at room temperature under a hydrogen atmosphere. Palladium-carbon was removed by filtration and the filtrate was concentrated. The residue was subjected to silica gel column chromatography, and ethyl 2-(4-hydroxyphenyl)-2-methylpropanoate (17.20 g, yield 87%) was obtained as a colorless oil from a fraction eluted with ethyl acetate-hexane (1:3, volume ratio).

¹H-NMR (CDCl₃)δ: 1.19 (3H, t, J=7.1 Hz), 1.55 (6H, s), 4.12 (2H, q, J=7.2 Hz), 5.26 (1H, s), 6.74-6.79 (2H, m), 7.18-7.23 (2H, m)

### Reference Example 84

To a mixture of (3-benzyloxyphenyl)methanol (22.09 g) and dichloroethane (250 ml) was added thionyl chloride (14.8 ml) at 0° C., and the mixture was stirred at room temperature for 3 hours. The reaction mixture was concentrated, and the residue was poured into aqueous sodium hydrogen carbonate and extracted with diethyl ether. The diethyl ether layer was washed with saturated aqueous sodium chloride solution, dried (MgSO₄) and concentrated to give a residue. A mixture of the obtained residue, sodium cyanide (5.32 g) and N,N-dimethylformamide (100 ml) was stirred overnight at 50° C. The reaction mixture was poured into water, and extracted with ethyl acetate. The ethyl acetate layer was washed with saturated aqueous sodium chloride solution, dried (MgSO₄) and concentrated. The residue was subjected to silica gel column chromatography, and (3-benzyloxyphenyl)acetonitrile (19.64 g, yield 85%) was obtained as a pale-yellow oily substance from a fraction eluted with ethyl acetate-hexane (1:3, volume ratio).

¹H-NMR (CDCl₃)δ: 3.72 (2H, s), 5.07 (2H, s), 6.89-6.96 (3H, m), 7.24-7.45 (6H, m).

### Reference Example 85

To a mixture of (3-benzyloxyphenyl)acetonitrile (19.64 g), methyl iodide (16.5 ml) and dimethyl sulfoxide (200 ml) was slowly added 50% aqueous sodium hydroxide solution (28.2 g) at 0° C., and the mixture was stirred at room temperature for 3 hours. The reaction mixture was poured into water, and extracted with ethyl acetate. The ethyl acetate layer was washed with saturated aqueous sodium chloride solution, dried (MgSO₄) and concentrated to give 2-(3-benzyloxyphenyl)-2-methylpropanenitrile (21.63 g, yield 98%) as a yellow oily substance.

¹H-NMR (CDCl₃)δ: 1.71 (6H, s), 5.08 (2H, s), 6.90-6.94 (1H, m) 7.05-7.11 (2H, m), 7.28-7.47 (6H, m).

### Reference Example 86

A mixture of 2-(3-benzyloxyphenyl)-2-methylpropanenitrile (21.63 g), potassium hydroxide (17.0 g) and ethylene glycol (150 ml) was stirred at 120° C. for 2 days. The reaction mixture was poured into ice water, acidified by adding hydrochloric acid, and extracted with ethyl acetate. The ethyl acetate layer was washed with saturated aqueous sodium chloride solution, dried (MgSO₄) and concentrated to give 2-(3-benzyloxyphenyl)-2-methylpropanoic acid (20.68 g, yield 89%) as yellow crystals. melting point: 114-116° C.

¹H-NMR (CDCl₃)δ: 1.58 (6H, s), 5.05 (2H, s), 6.85-6.89 (2H, m), 6.98-7.05 (2H, m), 7.23-7.46 (6H, m).

### Reference Example 87

A mixture of 2-(3-benzyloxyphenyl)-2-methylpropanoic acid (20.68 g), potassium carbonate (10.6 g), methyl iodide (7.1 ml) and N,N-dimethylformamide (160 ml) was stirred at room temperature for 2 hours. The reaction mixture was poured into water, and extracted with ethyl acetate. The ethyl acetate layer was washed with saturated aqueous sodium chloride solution, dried (MgSO₄) and concentrated. The residue was subjected to silica gel column chromatography, and methyl 2-(3-benzyloxyphenyl)-2-methylpropanoate (19.62 g, yield 90%) was obtained as a pale-yellow oily substance from a fraction eluted with ethyl acetate-hexane (1:5, volume ratio).

¹H-NMR (CDCl₃)δ: 1.56 (6H, s), 3.63 (3H, s), 5.05 (2H, s), 6.84-6.97 (3H, m), 7.22-7.46 (6H, m)

### Reference Example 88

A mixture of methyl 2-(3-benzyloxyphenyl)-2-methylpropanoate (19.62 g), 5% palladium-carbon (2.0 g) and ethanol (100 ml) was stirred overnight at room temperature under a hydrogen atmosphere. Palladium-carbon was removed by filtration and the filtrate was concentrated. The residue was subjected to silica gel column chromatography, and methyl 2-(3-hydroxyphenyl)-2-methylpropanoate (12.32 g, yield 92%) was obtained as a pale-yellow oily substance from a fraction eluted with ethyl acetate-hexane (1:3, volume ratio).

¹H-NMR (CDCl₃)δ: 1.56 (6H, s), 3.66 (3H, s), 5.35 (1H, s), 6.72 (1H, ddd, J=8.1, 2.4, 1.0 Hz), 6.83 (1H, t, J=2.1 Hz), 6.89 (1H, ddd, J=7.8, 1.7, 1.0 Hz), 7.19 (1H, t, J=7.9 Hz)

### Reference Example 89

A mixture of 3,4-dihydroxybenzaldehyde (25.30 g), potassium carbonate (15.20 g), benzyl bromide (21.7 ml) and N,N-dimethylformamide (250 ml) was stirred overnight at room temperature. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and 3-benzyloxy-4-hydroxybenzaldehyde (24.62 g, yield 59%) was obtained from a fraction eluted with ethyl acetate-hexane-chloroform (3:10:12, volume ratio). The crystals were recrystallized from ethanol. melting point: 123-124° C.

¹H-NMR (CDCl₃)δ: 5.21 (2H, s), 5.79 (1H, s), 7.04 (1H, d, J=8.3 Hz), 7.38-7.47 (7H, m), 9.84 (1H, s).

### Reference Example 90

A mixture of 3-benzyloxy-4-hydroxybenzaldehyde (10.60 g), potassium carbonate (12.84 g), chloromethyl methyl ether (5.2 ml) and N,N-dimethylformamide (150 ml) was stirred overnight at room temperature. The reaction mixture was poured into dilute hydrochloric acid, and extracted with toluene. The toluene layer was washed with saturated aqueous sodium chloride solution, dried (MgSO₄) and concentrated. The residue was subjected to silica gel column chromatography, and a colorless oil was obtained from a fraction eluted with ethyl acetate-hexane (1:2, volume ratio). To a mixture of the obtained oily substance, ethyl diethylphosphonoacetate (12.38 g) and N,N-dimethylformamide (90 ml) was added sodium hydride (60%, in oil, 2.43 mg) at 0° C. and the mixture was stirred overnight at room temperature. The reaction mixture was poured into water, and extracted with ethyl acetate. The ethyl acetate layer was washed with saturated aqueous sodium chloride solution, dried (MgSO₄) and concentrated. The residue was subjected to silica gel column chromatography, and ethyl (E)-3-[3-benzyloxy-4-(methoxymethoxy)phenyl]propenoate (13.48 g, yield 85%) was obtained as a pale-yellow oily substance from a fraction eluted with ethyl acetate-hexane (1:2, volume ratio).

¹H-NMR (CDCl₃)δ: 1.33 (3H, t, J=7.1 Hz), 3.53 (3H, s), 4.25 (2H, q, J=7.1 Hz), 5.19 (2H, s), 5.25 (2H, s), 6.30 (1H, d, J=15.9 Hz), 6.90 (1H, d, J=8.5 Hz), 7.10 (1H, dd, J=8.3, 2.2 Hz), 7.29-7.44 (5H, m), 7.59 (1H, d, J=15.9 Hz), 9.84 (1H, s).

### Reference Example 91

A mixture of ethyl (E)-3-[3-benzyloxy-4-(methoxymethoxy)phenyl]propenoate (13.48 g), 5% palladium-carbon (1.35 g) and ethanol (60 ml) was stirred overnight at room temperature under a hydrogen atmosphere. Palladium-carbon was removed by filtration and the filtrate was concentrated to give a residue. A mixture of the obtained residue, potassium carbonate (10.88 g), benzyl bromide (5.1 ml) and N,N-dimethylformamide (50 ml) was stirred overnight at room temperature. The reaction mixture was poured into water, and extracted with ethyl acetate. The ethyl acetate layer was washed with saturated aqueous sodium chloride solution, dried (MgSO₄) and concentrated. The residue was subjected to silica gel column chromatography, and ethyl 3-[3-benzyloxy-4-(methoxymethoxy)phenyl]propionate (9.46 g, yield 70%) was obtained as a colorless oil from a fraction eluted with ethyl acetate-hexane (1:2, volume ratio).

¹H-NMR (CDCl₃)δ: 1.23 (3H, t, J=7.2 Hz), 2.58 (2H, d, J=7.8 Hz), 2.87 (2H, t, J=7.8 Hz), 3.52 (3H, s), 4.12 (2H, q, J=7.1 Hz), 5.12 (2H, s), 5.21 (2H, s), 6.76 (1H, dd, J=8.3, 2.0 Hz), 6.83 (1H, d, J=8.1 Hz), 6.99 (1H, d, J=2.2 Hz), 7.27-7.44 (5H, m).

### Reference Example 92

To a mixture of ethyl 3-[3-benzyloxy-4-(methoxymethoxy)phenyl]propionate (9.46 g) and ethanol (100 ml) was added hydrochloric acid (3 drops) with a pipette, and the mixture was stirred at 80° C. for 1 hour. The reaction solution was concentrated. The residue was subjected to silica gel column chromatography, and ethyl 3-(3-benzyloxy-4-hydroxyphenyl)propionate (8.13 g, yield 99%) was obtained as colorless crystals from a fraction eluted with ethyl acetate-hexane (1:2, volume ratio). melting point: 60-61° C.

### Reference Example 93

A mixture of ethyl (E)-3-(2-benzyloxy-3-methoxyphenyl)propenoate (6.65 g), 5% palladium-carbon (2.46 g) and tetrahydrofuran (100 ml) was stirred overnight at room temperature under a hydrogen atmosphere. Palladium-carbon was removed by filtration and the filtrate was concentrated. The residue was subjected to silica gel column chromatography, and ethyl 3-(2-hydroxy-3-methoxyphenyl)propionate (5.86 g, yield 88%) was obtained as a colorless oil from a fraction eluted with ethyl acetate-hexane (1:4, volume ratio).

¹H-NMR (CDCl₃)δ: 1.23 (3H, t, J=7.0 Hz), 2.58-2.69 (2H, m), 2.90-3.01 (2H, m), 3.88 (3H, s), 4.13 (2H, q, J=7.0 Hz), 5.84 (1H, s), 6.72-6.78 (3H, m).

### Reference Example 94

A mixture of 2-hydroxy-5-methoxybenzaldehyde (10.25 g), benzyl bromide (8.1 ml), potassium carbonate (13.93 g) and N,N-dimethylformamide (100 ml) was stirred overnight at room temperature. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and a colorless oil was obtained from a fraction eluted with ethyl acetate-hexane (1:4, volume ratio). To a mixture of the colorless oil, ethyl diethylphosphonoacetate (15.66 g) and N,N-dimethylformamide (100 ml) was added sodium hydride (60%, in oil, 2.73 g) at 0° C. and the mixture was stirred overnight at room temperature. The reaction mixture was poured into water, and extracted with ethyl acetate. The ethyl acetate layer was washed with dilute hydrochloric acid and then with saturated aqueous sodium chloride solution, dried (MgSO₄) and concentrated. The residue was subjected to silica gel column chromatography, and ethyl (E)-3-(2-benzyloxy-5-methoxyphenyl)propenoate (16.58 g, yield 79%) was obtained as a colorless oil from a fraction eluted with ethyl acetate-hexane (1:4, volume ratio).

¹H-NMR (CDCl₃)δ: 1.33 (3H, t, J=7.0 Hz), 3.78 (3H, s), 4.26 (2H, q, J=7.0 Hz), 5.11 (2H, s), 6.49 (1H, d, J=16.0 Hz), 6.80-6.94 (2H, m), 7.04-7.11 (1H, m), 7.26-7.48 (5H, m), 8.06 (1H, d, J=16.0 Hz).

### Reference Example 95

A mixture of ethyl (E)-3-(2-benzyloxy-5-methoxyphenyl)propenoate (6.83 g), 5% palladium-carbon (1.11 g) and tetrahydrofuran (100 ml) was stirred overnight at room temperature under a hydrogen atmosphere. Palladium-carbon was removed by filtration and the filtrate was concentrated. The residue was subjected to silica gel column chromatography, and ethyl 3-(2-hydroxy-5-methoxyphenyl)propionate (4.54 g, yield 92%) was obtained as a colorless oil from a fraction eluted with ethyl acetate-hexane (1:4, volume ratio).

¹H-NMR (CDCl₃)δ: 1.23 (3H, t, J=7.2 Hz), 2.68-2.74 (2H, m), 2.83-2.89 (2H, m), 3.74 (3H, s), 4.13 (2H, q, J-7.2 Hz), 6.62-6.70 (2H, m), 6.83 (1H, d, J=8.4 Hz), 6.95-6.98 (1H, br s).

### Reference Example 96

A mixture of 2-hydroxy-4-methoxybenzaldehyde (25.16 g), benzyl bromide (20 ml), potassium carbonate (25.03 g) and N,N-dimethylformamide (300 ml) was stirred overnight at room temperature. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and 2-benzyloxy-4-methoxybenzaldehyde (37.18 g, yield 93%) was obtained as a colorless oil from a fraction eluted with ethyl acetate-hexane (1:4, volume ratio).

¹H-NMR (CDCl₃)δ: 3.86 (3H, s), 5.17 (2H, s), 6.50-6.62 (2H, m), 7.24-7.50 (5H, m), 7.85 (1H, d, J=8.4 Hz), 10.39 (1H, s).

### Reference Example 97

To a mixture of 2-benzyloxy-4-methoxybenzaldehyde (5.00 g), ethyl diethylphosphonoacetate (4.75 g) and N,N-dimethylformamide (50 ml) was added sodium hydride (60%, in oil, 0.84 g) at 0° C., and the mixture was stirred overnight at room temperature. The reaction mixture was poured into water, and extracted with ethyl acetate. The ethyl acetate layer was washed with dilute hydrochloric acid and then with saturated aqueous sodium chloride solution, dried (MgSO₄) and concentrated. The residue was subjected to silica gel column chromatography, and ethyl (E)-3-(2-benzyloxy-4-methoxyphenyl)propenoate (5.48 g, yield 85%) was obtained as a colorless oil from a fraction eluted with ethyl acetate-hexane (1:4, volume ratio).

¹H-NMR (CDCl₃)δ: 1.32 (3H, t, J=6.8 Hz), 3.80 (3H, s), 4.23 (2H, q, J=6.8 Hz), 5.15 (2H, s), 6.37-6.56 (3H, m), 7.24-7.53 (6H, m), 8.00 (1H, d, J=16.2 Hz).

### Reference Example 98

A mixture of ethyl (E)-3-(2-benzyloxy-4-methoxyphenyl)propenoate (5.45 g), 5% palladium-carbon (1.16 g) and tetrahydrofuran (100 ml) was stirred overnight at room temperature under a hydrogen atmosphere. Palladium-carbon was removed by filtration and the filtrate was concentrated. The residue was subjected to silica gel column chromatography, and ethyl 3-(2-hydroxy-4-methoxyphenyl)propionate (3.80 g, yield 97%) was obtained as a colorless oil from a fraction eluted with ethyl acetate-hexane (1:4, volume ratio).

¹H-NMR (CDCl₃)δ: 1.24 (3H, t, J=7.0 Hz), 2.57-2.68 (2H, m), 2.77-2.88 (2H, m), 3.76 (3H, s), 4.15 (2H, q, J=7.0 Hz), 6.40-6.52 (2H, m), 6.97 (1H, d, J=8.0 Hz), 7.58 (1H, br s).

### Reference Example 99

To a solution of 2-benzyloxy-4-methoxybenzaldehyde (13.15 g) in tetrahydrofuran (100 ml) was added lithium aluminum hydride (1.50 g) at 0° C., and the mixture was stirred at room temperature for 1 hour. To the reaction mixture was added sodium sulfate 10 hydrate (15.09 g), and the mixture was stirred at room temperature for 1 hour. The precipitate was removed by filtration and the filtrate was concentrated. The residue was subjected to silica gel column chromatography, and 2-benzyloxy-4-methoxybenzyl alcohol (12.84 g, yield 97%) was obtained as a colorless oil from a fraction eluted with ethyl acetate.

¹H-NMR (CDCl₃)δ: 2.19 (1H, br t), 3.79 (3H, s), 4.66 (2H, d, J=5.8 Hz), 5.09 (2H, s), 6.44-6.56 (2H m), 7.16-7.46 (6H, m).

### Reference Example 100

To a mixture of 2-benzyloxy-4-methoxybenzyl alcohol (12.25 g), acetone cyanohydrin (5.70 g), triphenylphosphine (20.03 g) and tetrahydrofuran (200 ml) was dropwise added a 40% toluene solution (32.75 g) of diethyl azodicarboxylate at room temperature, and the mixture was stirred overnight. The reaction solution was concentrated. The residue was subjected to silica gel column chromatography, and (2-benzyloxy-4-methoxyphenyl)acetonitrile (10.34 g, yield 81%) was obtained as a colorless oil from a fraction eluted with ethyl acetate-hexane (1:4, volume ratio).

¹H-NMR (CDCl₃)δ: 3.65 (2H, s), 3.79 (3H, s), 5.08 (2H, s), 6.43-6.56 (2H, m), 7.22-7.48 (6H, m).

### Reference Example 101

A mixture of (2-benzyloxy-4-methoxyphenyl)acetonitrile (10.34 g), 8N aqueous sodium hydroxide solution (50 ml) and ethanol (200 ml) was stirred under reflux overnight. After cooling, the reaction mixture was acidified by slowly adding conc. hydrochloric acid (350 ml). After concentration, the residue was dissolved in ethyl acetate. The obtained ethyl acetate solution was washed with saturated aqueous sodium chloride solution, dried (MgSO₄) and concentrated. A mixture of the residue, a 10% solution (200 ml) of hydrochloric acid in methanol and methanol (200 ml) was stirred overnight at room temperature. After concentration, the residue was dissolved in ethyl acetate. The obtained ethyl acetate solution was washed with saturated aqueous sodium chloride solution, dried (MgSO₄) and concentrated. The residue was subjected to silica gel column chromatography, and methyl (2-benzyloxy-4-methoxyphenyl)acetate (9.35 g, yield 80%) was obtained as a colorless oil from a fraction eluted with ethyl acetate-hexane (1:4, volume ratio).

¹H-NMR (CDCl₃)δ: 3.61 (2H, s), 3.63 (3H, s), 3.78 (3H, s), 5.06 (2H, s), 6.43-6.54 (2H, m), 7.11 (1H, d, J=8.0 Hz), 7.24-7.46 (5H, m).

### Reference Example 102

A mixture of methyl (2-benzyloxy-4-methoxyphenyl)acetate (9.35 g), 5% palladium-carbon (1.44 g) and tetrahydrofuran (100 ml) was stirred overnight at room temperature under a hydrogen atmosphere. Palladium-carbon was removed by filtration and the filtrate was concentrated. The residue was subjected to silica gel column chromatography, and methyl (2-hydroxy-4-methoxyphenyl)acetate (6.11 g, yield 95%) was obtained as a colorless oil from a fraction eluted with ethyl acetate-hexane (1:4, volume ratio).

¹H-NMR (CDCl₃)δ: 3.62 (2H, s), 3.75 (3H, s), 3.77 (3H, s), 6.45 (1H, dd, J=2.4, 8.4 Hz), 6.53 (1H, d, J=2.4 Hz), 6.98 (1H, d, J=8.4 Hz), 7.62 (1H, s).

### Reference Example 103

A mixture of 2-hydroxy-3-methoxybenzaldehyde (8.50 g), benzyl bromide (6.7 ml), potassium carbonate (11.66 g) and N,N-dimethylformamide (100 ml) was stirred overnight at room temperature. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and 2-benzyloxy-3-methoxybenzaldehyde (13.08 g, yield 97%) was obtained as a colorless oil from a fraction eluted with ethyl acetate-hexane (1:4, volume ratio).

¹H-NMR (CDCl₃)δ: 3.95 (3H, s), 5.18 (2H, s), 7.10-7.21 (2H, m), 7.32-7.43 (6H, m), 10.23 (1H, s).

### Reference Example 104

To a mixture of 2-benzyloxy-3-methoxybenzaldehyde (5.51 g), ethyl diethylphosphonoacetate (6.12 g) and N,N-dimethylformamide (50 ml) was added sodium hydride (60%, in oil, 1.03 g) at 0° C., and the mixture was stirred overnight at room temperature. The reaction mixture was poured into water, and extracted with ethyl acetate. The ethyl acetate layer was washed with dilute hydrochloric acid and then with saturated aqueous sodium chloride solution, dried (MgSO₄) and concentrated. The residue was subjected to silica gel column chromatography, and ethyl (E)-3-(2-benzyloxy-3-methoxyphenyl)propenoate (6.68 g, yield 94%) was obtained as a colorless oil from a fraction eluted with ethyl acetate-hexane (1:4, volume ratio).

¹H-NMR (CDCl₃)δ: 1.33 (3H, t, J=7.0 Hz), 3.90 (3H, s), 4.24 (2H, q, J=7.0 Hz), 5.02 (2H, s), 6.38 (1H, d, J=16.4 Hz), 6.92-7.18 (3H, m), 7.28-7.52 (5H, m), 7.98 (1H, d, J=16.4 Hz).

### Reference Example 105

A mixture of [3-(benzyloxy)-1-methyl-1H-pyrazol-5-yl]acetonitrile (5.08 g), 6N aqueous sodium hydroxide solution (30 ml), tetrahydrofuran (30 ml) and methanol (30 ml) was stirred at 80° C. for 2.5 days. The reaction mixture was neutralized with 1N hydrochloric acid, and extracted with ethyl acetate. The ethyl acetate layer was washed with saturated aqueous sodium chloride solution, dried (MgSO₄) and concentrated to give a brown oily substance. To a mixture of the obtained oily substance, potassium carbonate (6.12 g) and N,N-dimethylformamide (230 ml) was added methyl iodide (2.76 ml) at room temperature, and the mixture was stirred overnight. The reaction mixture was poured into saturated aqueous ammonium chloride solution, and extracted with ethyl acetate. The ethyl acetate layer was washed with saturated aqueous sodium chloride solution, dried (MgSO₄) and concentrated. The residue was subjected to silica gel column chromatography, and methyl [3-(benzyloxy)-1-methyl-1H-pyrazol-5-yl]acetate (1.60 g, yield 28%) was obtained as a yellow oily substance from a fraction eluted with ethyl acetate-hexane (1:6, volume ratio).

¹H-NMR (CDCl₃)δ: 3.60 (2H, s), 3.68 (3H, s), 3.72 (3H, s), 5.15 (2H, s), 5.62 (1H, s), 7.26-7.46 (5H, m).

### Reference Example 106

A mixture of methyl [3-(benzyloxy)-1-methyl-1H-pyrazol-5-yl]acetate (1.60 g), 5% palladium-carbon (320 mg) and ethanol (100 ml) was stirred at room temperature for 2.5 hours under a hydrogen atmosphere. Palladium-carbon was removed by filtration and the filtrate was concentrated to give methyl (3-hydroxy-1-methyl-1H-pyrazol-5-yl)acetate (1.02 g, yield 97%) as a yellow solid. The crystals were recrystallized from ethyl acetate-hexane to give colorless crystals. melting point: 147-148° C.

### Reference Example 107

To a mixture of 3-(1-benzyl-3-ethoxy-1H-pyrazol-4-yl)-1-propanol (6.75 g), ethyl 2-(3-hydroxyphenoxy)-2-methylpropanoate (6.39 g), tributylphosphine (12.9 ml) and tetrahydrofuran (1.00 L) was added 1,1′-azodicarbonyldipiperidine (13.1 g) at room temperature, and the mixture was stirred overnight. The reaction solution was concentrated. The residue was subjected to silica gel column chromatography, and ethyl 2-{3-[3-(1-benzyl-3-ethoxy-1H-pyrazol-4-yl)propoxy]phenoxy}-2-methylpropanoate (9.47 g, yield 78%) was obtained as a pale-yellow oily substance from a fraction eluted with ethyl acetate-hexane (1:6, volume ratio).

¹H-NMR (CDCl₃)δ: 1.24 (3H, t, J=7.2 Hz), 1.35 (3H, t, J=6.9 Hz), 1.59 (6H, s), 1.92-2.03 (2H, m), 2.45-2.55 (2H, m), 3.86-3.94 (2H, m), 4.18-4.28 (4H, m), 5.07 (2H, s), 6.35-6.44 (2H, m), 6.49-6.54 (1H, m), 6.96 (1H, s), 7.06-7.12 (1H, m), 7.14-7.18 (2H, m), 7.26-7.36 (3H, m).

### Reference Example 108

A mixture of 1-benzyl-4-[3-(1,3-dioxolan-2-yl)propyl]-1H-pyrazol-3-ol (21.8 g), diethylsulfuric acid (17.3 ml), potassium carbonate (16.7 g) and N,N-dimethylformamide (150 ml) was stirred overnight at room temperature. The reaction mixture was poured into saturated aqueous ammonium chloride solution, and extracted with ethyl acetate. The ethyl acetate layer was washed with saturated aqueous sodium chloride solution, dried (MgSO₄) and concentrated. The residue was subjected to silica gel column chromatography, and 1-benzyl-4-[3-(1,3-dioxolan-2-yl)propyl]-3-ethoxy-1H-pyrazole (19.5 g, yield 82%) was obtained as a yellow oily substance from a fraction eluted with ethyl acetate-hexane (1:4, volume ratio).

¹H-NMR (CDCl₃)δ: 1.36 (3H, t, J=6.9 Hz), 1.57-1.74 (4H, m), 2.32-2.39 (2H, m), 3.80-3.98 (4H, m), 4.22 (2H, q, J=6.9 Hz), 4.82-4.87 (1H, m), 5.07 (2H, s), 6.93 (1H, s), 7.13-7.17 (2H, m), 7.23-7.35 (3H, m).

### Reference Example 109

To a mixture of ethyl 4-(1-benzyl-3-ethoxy-1H-pyrazol-4-yl)-1-butanol (1.50 g), ethyl 2-(3-hydroxyphenoxy)-2-methylpropanoate (1.35 g), tributylphosphine (2.73 ml) and tetrahydrofuran (110 ml) was added 1,1′-azodicarbonyldipiperidine (2.76 g) at room temperature, and the mixture was stirred overnight. The reaction solution was concentrated. The residue was subjected to silica gel column chromatography, and ethyl 2-{3-[4-(1-benzyl-3-ethoxy-1H-pyrazol-4-yl)butoxy]phenoxy}-2-methylpropanoate (1.33 g, yield 52%) was obtained as a colorless oil from a fraction eluted with ethyl acetate-hexane (1:6, volume ratio).

¹H-NMR (CDCl₃)δ: 1.24 (3H, t, J=7.0 Hz), 1.37 (3H, t, J=7.0 Hz), 1.48-1.87 (4H, m), 1.59 (6H, s), 2.33-2.43 (2H, m), 3.86-3.95 (2H, m), 4.16-4.29 (4H, m), 5.09 (2H, s), 6.34-6.44 (2H, m), 6.48-6.56 (1H, m), 6.95 (1H, s), 7.04-7.20 (3H, m), 7.24-7.39 (3H, m).

### Reference Example 110

To a solution of potassium tert-butoxide (3.79 g) in 1,2-dimethoxyethane (17 ml) was dropwise added a solution of toluenesulfonylmethyl isocyanide (3.29 g) in 1,2-dimethoxyethane (17 ml) at −78° C. Then a solution of 5-(benzyloxy)-2-methoxybenzaldehyde (3.90 g) in 1,2-dimethoxyethane (50 ml) was dropwise added at the same temperature, and the reaction mixture was warmed to room temperature. The mixture was stirred at room temperature for 1 hour and methanol (85 ml) was added. The reaction mixture was heated until reflux and the mixture was stirred at said temperature for 2 hours. Saturated aqueous ammonium chloride solution was added to the reaction mixture, and extracted with ethyl acetate. The ethyl acetate layer was washed with saturated aqueous sodium chloride solution, dried (MgSO₄) and concentrated. The residue was subjected to silica gel column chromatography, and [5-(benzyloxy)-2-methoxyphenyl]acetonitrile (3.63 g, yield 89%) was obtained as a pale-yellow oily substance from a fraction eluted with ethyl acetate-hexane (1:6, volume ratio).

¹H-NMR (CDCl₃)δ: 3.66 (2H, s), 3.81 (3H, s), 5.02 (2H, s), 6.79 (1H, d, J=9.0 Hz), 6.88 (1H, dd, J=2.7, 9.0 Hz), 7.03 (1H, d, J=2.7 Hz), 7.28-7.44 (m, 5H).

### Reference Example 111

A mixture of [5-(benzyloxy)-2-methoxyphenyl]acetonitrile (3.63 g), 6N aqueous sodium hydroxide solution (40 ml), tetrahydrofuran (40 ml) and methanol (40 ml) was stirred at 80° C. for 3 days. The reaction mixture was neutralized with 1N hydrochloric acid, and extracted with ethyl acetate. The ethyl acetate layer was washed with saturated aqueous sodium chloride solution, dried (MgSO₄) and concentrated to give a pale-yellow solid. To a mixture of the obtained solid, potassium carbonate (3.95 g) and N,N-dimethylformamide (478 ml) was added methyl iodide (1.78 ml) at room temperature, and the mixture was stirred overnight. Dilute hydrochloric acid was added to the reaction mixture, and the mixture was extracted with ethyl acetate. The ethyl acetate layer was washed with saturated aqueous sodium chloride solution, dried (MgSO₄) and concentrated. The residue was subjected to silica gel column chromatography, and methyl [5-(benzyloxy)-2-methoxyphenyl]acetate (3.76 g, yield 92%) was obtained from a fraction eluted with ethyl acetate-hexane (1:4, volume ratio) as a brown solid. The crystals were recrystallized from ethyl acetate-hexane to give colorless crystals. melting point: 74-75° C.

### Reference Example 112

A mixture of methyl [5-(benzyloxy)-2-methoxyphenyl]acetate (3.61 g), 5% palladium-carbon (800 mg) and ethanol (150 ml) was stirred at room temperature for 4.5 hours under a hydrogen atmosphere. Palladium-carbon was removed by filtration and the filtrate was concentrated. The residue was subjected to silica gel column chromatography, and methyl (5-hydroxy-2-methoxyphenyl)acetate (2.40 g, yield 97%) was obtained as a pale-yellow oily substance from a fraction eluted with ethyl acetate-hexane (1:2, volume ratio).

¹H-NMR (CDCl₃)δ: 3.58 (2H, s), 3.70 (3H, s), 3.75 (3H, s), 5.21 (1H, s), 6.66-6.76 (3H, m).

### Reference Example 113

To a mixture of ethyl 2-{3-[3-(1-benzyl-3-ethoxy-1H-pyrazol-4-yl)propoxy]phenoxy}-2-methylpropanoate (9.47 g), 5% palladium-carbon (10.0 g) and ethanol (200 ml) was added formic acid (65 ml) and the mixture was stirred overnight while heating under reflux. Palladium-carbon was removed by filtration and the filtrate was concentrated. The residue was subjected to silica gel column chromatography, and ethyl 2-{3-[3-(3-ethoxy-1H-pyrazol-4-yl)propoxy]phenoxy}-2-methylpropanoate (5.10 g, yield 69%) was obtained as a yellow oily substance from a fraction eluted with ethyl acetate-hexane (1:1, volume ratio).

¹H-NMR (CDCl₃)δ: 1.25 (3H, t, J=7.0 Hz), 1.37 (3H, t, J=6.8 Hz), 1.60 (6H, s), 1.91-2.09 (2H, m), 2.48-2.60 (2H, m), 3.85-3.96 (2H, m), 4.16-4.30 (4H, m), 6.34-6.45 (2H, m), 6.50-6.58 (1H, m), 7.04-7.17 (2H, m).

### Reference Example 114

A mixture of ethyl 3-(3-ethoxy-1H-pyrazol-4-yl)propanoate (7.65 g), sodium hydride (60%, in oil, 1.16 g) and N,N-dimethylformamide (120 ml) was stirred at room temperature for 30 minutes, and 2-fluoropyridine (2.48 ml) was added. The mixture was stirred at 100° C. overnight. To the reaction mixture was added dilute hydrochloric acid, and the mixture was extracted with ethyl acetate. The ethyl acetate layer was washed with saturated aqueous sodium chloride solution, dried (MgSO₄) and concentrated. The residue was subjected to silica gel column chromatography, and ethyl 3-[3-ethoxy-1-(2-pyridinyl)-1H-pyrazol-4-yl]propanoate (1.52 g, yield 22%) was obtained as a colorless oil from a fraction eluted with ethyl acetate-hexane (1:6, volume ratio).

¹H-NMR (CDCl₃)δ: 1.26 (3H, t, J=7.2 Hz), 1.43 (3H, t, J=7.2 Hz), 2.57-2.65 (2H, m), 2.70-2.78 (2H, m), 4.14 (2H, q, J=7.2 Hz), 4.34 (2H, q, J=7.2 Hz), 6.98-7.06 (1H, m), 7.66-7.74 (2H, m), 8.16 (1H, s), 8.27-8.31 (1H, m).

### Reference Example 115

To a solution of ethyl 3-[3-ethoxy-1-(2-pyridinyl)-1H-pyrazol-4-yl]propanoate (2.90 g) in tetrahydrofuran (100 ml) was dropwise added a 0.93 M solution (22.0 ml) of diisobutylaluminum hydride in hexane at 0° C., and the mixture was stirred at room temperature for 2 hours. The reaction mixture was cooled to 0° C. and a 0.93 M solution (11.0 ml) of diisobutylaluminum hydride in hexane was added dropwise. The reaction mixture was warmed to room temperature and the mixture was stirred for 1 hour. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and 3-[3-ethoxy-1-(2-pyridinyl)-1H-pyrazol-4-yl]-1-propanol (2.41 g, yield 97%) was obtained as a colorless oil from a fraction eluted with ethyl acetate-hexane (1:1, volume ratio).

¹H-NMR (CDCl₃)δ: 1.44 (3H, t, J=7.2 Hz), 1.73-1.90 (3H, m), 2.49-2.56 (2H, m), 3.64-3.71 (2H, m), 4.37 (2H, q, J=7.2 Hz), 6.98-7.08 (1H, m), 7.67-7.75 (2H, m), 8.16 (1H, s), 8.28-8.32 (1H, m).

### Reference Example 116

To a mixture of 2-(1,3-dioxolan-2-yl)ethyltetraphenylphosphonium bromide (53.2 g) and N,N-dimethylformamide (500 ml) was added sodium hydride (60%, in oil, 4.80 g) at 0° C. The reaction mixture was stirred at room temperature for 30 minutes and a solution of 1-benzyl-3-(benzyloxy)-1H-pyrazole-4-carbaldehyde (28.9 g) in N,N-dimethylformamide (100 ml) was added. The mixture was stirred at room temperature overnight, and at 70° C. for 5 hours. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and a yellow oily substance was obtained from a fraction eluted with ethyl acetate-hexane (1:6, volume ratio). A mixture of the obtained oily substance, 5% palladium-carbon (3.80 g) and ethanol (500 ml) was stirred overnight at room temperature under a hydrogen atmosphere. Palladium-carbon was removed by filtration and the filtrate was concentrated. The residue was subjected to silica gel column chromatography, and 1-benzyl-4-[3-(1,3-dioxolan-2-yl)propyl]-1H-pyrazol-3-ol (21.8 g, yield 76%) was obtained as a white solid from a fraction eluted with ethyl acetate-hexane (1:1, volume ratio). The crystals were recrystallized from ethyl acetate-hexane to give colorless crystals. melting point: 93-94° C.

### Reference Example 117

A mixture of 1-benzyl-4-[3-(1,3-dioxolan-2-yl)propyl]-3-ethoxy-1H-pyrazole (22.0 g), 1N hydrochloric acid (150 ml), ethanol (150 ml) and tetrahydrofuran (150 ml) was stirred at room temperature for 2.5 hours, and at 50° C. for 3 hours. Saturated aqueous ammonium chloride solution was added to the reaction mixture, and the mixture was extracted with ethyl acetate. The ethyl acetate layer was washed with saturated aqueous sodium chloride solution, dried (MgSO₄) and concentrated. The residue was subjected to silica gel column chromatography, and 4-(1-benzyl-3-ethoxy-1H-pyrazol-4-yl)butanal (10.1 g, yield 53%) was obtained as a colorless oil from a fraction eluted with ethyl acetate-hexane (1:4, volume ratio).

¹H-NMR (CDCl₃)δ: 1.36 (3H, t, J=6.9 Hz), 1.79-1.91 (2H, m), 2.32-2.48 (4H, m), 4.22 (2H, q, J=6.9 Hz), 5.07 (2H, s), 6.93 (1H, s), 7.13-7.18 (2H, m), 7.24-7.36 (3H, m), 9.73 (1H, s).

### Reference Example 118

To a solution of 4-(1-benzyl-3-ethoxy-1H-pyrazol-4-yl)butanal (10.1 g) in ethanol (185 ml) was added sodium borohydride (1.54 g) at room temperature, and the mixture was stirred overnight. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and 4-(1-benzyl-3-ethoxy-1H-pyrazol-4-yl)-1-butanol (9.44 g, yield 93%) was obtained as a colorless oil from a fraction eluted with ethyl acetate-hexane (1:2, volume ratio).

¹H-NMR (CDCl₃)δ: 1.37 (3H, t, J=7.0 Hz), 1.52-1.69 (4H, m) 2.29-2.41 (2H, m), 3.60-3.71 (2H, brm), 4.23 (2H, q, J=7.0 Hz), 5.08 (2H, s), 6.94 (1H, s), 7.13-7.21 (2H, m), 7.22-7.39 (3H, m).

### Reference Example 119

To a mixture of ethyl 2-{3-[4-(1-benzyl-3-ethoxy-1H-pyrazol-4-yl)butoxy]phenoxy}-2-methylpropanoate (950 mg), 5% palladium-carbon (950 mg) and ethanol (10 ml) was added formic acid (3.3 ml), and the mixture was stirred while heating under reflux for 3 hours. Palladium-carbon was removed by filtration and the filtrate was concentrated. The residue was subjected to silica gel column chromatography, and ethyl 2-{3-[4-(3-ethoxy-1H-pyrazol-4-yl)butoxy]phenoxy}-2-methylpropanoate (740 mg, yield 93%) was obtained as a colorless oil from a fraction eluted with ethyl acetate-hexane (1:1, volume ratio).

¹H-NMR (CDCl₃)δ: 1.25 (3H, t, J=7.2 Hz), 1.39 (3, t, J=7.2 Hz), 1.59 (6H, s), 1.63-1.89 (4H, m), 2.38-2.46 (2H, m), 3.89-3.95 (2H, m), 4.18-4.28 (4H, m), 6.35-6.43 (2H, m), 6.49-6.55 (1H, m), 7.05-7.12 (1H, m), 7.15 (1H, s).

### Reference Example 120

To a mixture of 4-(1-benzyl-3-ethoxy-1H-pyrazol-4-yl)-1-butanol (1.50 g), methyl 3-(4-hydroxy-2-ethoxyphenyl)propanoate (1.35 g), tributylphosphine (2.73 ml) and tetrahydrofuran (110 ml) was added 1,1′-azodicarbonyldipiperidine (2.76 g) at room temperature, and the mixture was stirred for 2.5 days. The reaction solution was concentrated. The residue was subjected to silica gel column chromatography, and a yellow oily substance was obtained from a fraction eluted with ethyl acetate-hexane (1:6, volume ratio). To a mixture of the obtained oily substance, 5% palladium-carbon (1.80 g) and ethanol (18 ml) was added formic acid (6.0 ml) and the mixture was stirred while heating under reflux for 7 hours. Palladium-carbon was removed by filtration and the filtrate was concentrated. The residue was subjected to silica gel column chromatography, and methyl 3-{2-ethoxy-4-[4-(3-ethoxy-1H-pyrazol-4-yl)butoxy]phenyl}propanoate (0.86 g, yield 60%) was obtained as a brown oily substance from a fraction eluted with ethyl acetate-hexane (1:1, volume ratio).

¹H-NMR (CDCl₃)δ: 1.35-1.45 (6H, m), 1.62-1.90 (4H, m), 2.38-2.48 (2H, m), 2.53-2.64 (2H, m), 2.81-2.92 (2H, m), 3.66 (3H, s), 3.90-4.06 (4H, m), 4.21 (2H, q, J=7.0 Hz), 6.28-6.43 (2H, m), 6.94-7.04 (1H, m), 7.17 (1H, s).

### Reference Example 121

To a mixture of 4-(1-benzyl-3-ethoxy-1H-pyrazol-4-yl)-1-butanol (1.01 g), ethyl 3-(3-hydroxy-1-phenyl-1H-pyrazol-5-yl)propanoate (1.05 g), tributylphosphine (1.83 ml) and tetrahydrofuran (75 ml) was added 1,1′-azodicarbonyldipiperidine (1.85 g) at room temperature, and the mixture was stirred overnight. The reaction solution was concentrated. The residue was subjected to silica gel column chromatography, and a colorless oil was obtained from a fraction eluted with ethyl acetate-hexane (1:6, volume ratio). To a mixture of the obtained oily substance, 5% palladium-carbon (1.73 g) and ethanol (18 ml) was added formic acid (6 ml) and the mixture was stirred overnight while heating under reflux. Palladium-carbon was removed by filtration and the filtrate was concentrated. The residue was subjected to silica gel column chromatography, and ethyl 3-{3-[4-(3-ethoxy-1H-pyrazol-4-yl)butoxy]-1-phenyl-1H-pyrazol-5-yl}propanoate (900 mg, yield 57%) was obtained as a yellow oily substance from a fraction eluted with ethyl acetate-hexane (1:1, volume ratio).

¹H-NMR (CDCl₃)δ: 1.24 (3H, t, J=7.0 Hz), 1.39 (3H, t, J=7.0 Hz), 1.64-1.87 (4H, m), 2.36-2.47 (2H, m), 2.52-2.63 (2H, m), 2.88-2.99 (2H, m), 4.05-4.30 (6H, m), 5.65 (1H, s), 7.15 (1H, s), 7.28-7.50 (5H, m).

### Reference Example 122

A mixture of ethyl 3-(3-ethoxy-1H-pyrazol-4-yl)propanoate (5.00 g), 4-(trifluoromethyl)phenylboric acid (8.95 g), copper(II) acetate (6.42 g), pyridine (3.42 ml) and methylene chloride (120 ml) was stirred overnight at room temperature. The precipitate was removed by filtration, and the filtrate was concentrated. The residue was subjected to silica gel column chromatography, and ethyl 3-{3-ethoxy-1-[4-(trifluoromethyl)phenyl]-1H-pyrazol-4-yl}propanoate (2.41 g, yield 29%) was obtained from a fraction eluted with ethyl acetate-hexane (1:9, volume ratio) as colorless crystals. The crystals were recrystallized from ethyl acetate-hexane. melting point: 47-48° C.

### Reference Example 123

To a solution of ethyl 3-{3-ethoxy-1-[4-(trifluoromethyl)phenyl]-1H-pyrazol-4-yl}propanoate (4.31 g) in tetrahydrofuran (120 ml) was dropwise added a 0.93 M solution (39 ml) of diisobutylaluminum hydride in hexane at 0° C. and the mixture was stirred at room temperature overnight. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and 3-{3-ethoxy-1-[4-(trifluoromethyl)phenyl]-1H-pyrazol-4-yl}-1-propanol (3.68 g, yield 97%) was obtained as a colorless oil from a fraction eluted with ethyl acetate-hexane (1:2, volume ratio).

¹H-NMR (CDCl₃)δ: 1.44 (3H, t, J=7.0 Hz), 1.68-1.92 (3H, m), 2.48-2.59 (2H, m), 3.62-3.75 (2H, brm), 4.37 (2H, q, J=7.0 Hz), 7.58-7.70 (5H, m).

### Reference Example 124

To a mixture of 4-(1-benzyl-3-ethoxy-1H-pyrazol-4-yl)-1-butanol (2.00 g), triethylamine (1.22 ml) and tetrahydrofuran (70 ml) at room temperature was added methanesulfonyl chloride (677 μL), and the mixture was stirred overnight. Triethylamine (2.03 ml) and methanesulfonyl chloride (1.13 ml) were added to the reaction mixture at room temperature and the mixture was stirred at room temperature for 2 hours. The reaction mixture was poured into saturated aqueous sodium hydrogen carbonate and extracted with ethyl acetate. The ethyl acetate layer was washed with saturated aqueous sodium chloride solution, dried (MgSO₄) and concentrated. The residue was subjected to silica gel column chromatography, and 4-(1-benzyl-3-ethoxy-1H-pyrazol-4-yl)butyl methanesulfonate (2.46 g, yield 96%) was obtained as a yellow oily substance from a fraction eluted with ethyl acetate-hexane (1:2, volume ratio).

¹H-NMR (CDCl₃)δ: 1.36 (3H, t, J=6.9 Hz), 1.54-1.68 (2H, m), 1.70-1.82 (2H, m), 2.32-2.40 (2H, m), 2.98 (3H, s), 4.18-4.26 (4H, m), 5.07 (2H, s), 6.92 (1H, s), 7.14-7.19 (2H, m), 7.24-7.36 (3H, m).

### Reference Example 125

A mixture of ethyl 3-(3-ethoxy-1H-pyrazol-4-yl)propanoate (662 mg), sodium hydride (60%, in oil, 136 mg) and N,N-dimethylformamide (25 ml) was stirred at room temperature for 30 minutes, and a solution of 4-(1-benzyl-3-ethoxy-1H-pyrazol-4-yl)butyl methanesulfonate (1.00 g) in N,N-dimethylformamide (5 ml) was added. The mixture was stirred overnight at room temperature and the reaction mixture was poured into 0.1N aqueous hydrochloric acid solution. The mixture was extracted with ethyl acetate. The ethyl acetate layer was washed with saturated aqueous sodium chloride solution, dried (MgSO₄) and concentrated. The residue was subjected to silica gel column chromatography, and a colorless oil was obtained from a fraction eluted with ethyl acetate-hexane (1:2, volume ratio). To a mixture of the obtained oily substance, 5% palladium-carbon (1.00 g) and ethanol (10 ml) was added formic acid (3 ml) and the mixture was stirred while heating under reflux for 4 hours. Palladium-carbon was removed by filtration and the filtrate was concentrated. The residue was diluted with ethyl acetate and washed with saturated aqueous sodium hydrogen carbonate, and saturated aqueous sodium chloride solution. After drying (MgSO₄), the mixture was concentrated to give ethyl 3-{3-ethoxy-1-[4-(3-ethoxy-1H-pyrazol-4-yl)butyl]-1H-pyrazol-4-yl}propanoate (680 mg, yield 63%) as a colorless oil.

¹H-NMR (CDCl₃)δ: 1.23 (3H, t, J=6.9 Hz), 1.32-1.41 (6H, m), 1.44-1.56 (2H, m), 1.72-1.84 (2H, m), 2.33-2.40 (2H, m), 2.48-2.56 (2H, m), 2.61-2.68 (2H, m), 3.84-3.91 (2H, m), 4.10 (2H, q, J=6.9 Hz), 4.15-4.27 (4H, m), 6.96 (1H, s), 7.10 (1H, s).

### Reference Example 126

To a solution of potassium tert-butoxide (5.22 g) in 1,2-dimethoxyethane (300 ml) was dropwise added a solution of toluenesulfonylmethyl isocyanide (4.54 g) in 1,2-dimethoxyethane (30 ml) at −78° C. After stirring at the same temperature for 10 minutes, a solution of 3-(benzyloxy)-1-methyl-1H-pyrazole-4-carbaldehyde (4.79 g) in 1,2-dimethoxyethane (60 ml) was added dropwise. The reaction mixture was warmed to room temperature. Then methanol (120 ml) was added and stirred while heating under reflux for 2.5 hours. The reaction mixture was poured into saturated aqueous ammonium chloride solution, and extracted with ethyl acetate. The ethyl acetate layer was washed with saturated aqueous sodium chloride solution and dried (MgSO₄). The solvent was removed under reduced pressure and [3-(benzyloxy)-1-methyl-1H-pyrazol-5-yl]acetonitrile (5.08 g, quantitative) was obtained as a brown oily substance.

¹H-NMR (CDCl₃)δ: 3.67 (2H, s), 3.73 (3H, s), 5.16 (2H, s), 5.73 (1H, s), 7.27-7.48 (5H, m)

### Reference Example 127

A mixture of 6-methoxysalicylaldehyde (11.20 g), benzyl bromide (8.8 ml), potassium carbonate (15.29 g) and N,N-dimethylformamide (200 ml) was stirred overnight at room temperature. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and 2-benzyloxy-6-methoxybenzaldehyde (15.64 g, yield 88%) was obtained as a colorless oil from a fraction eluted with ethyl acetate-hexane (1:4, volume ratio).

¹H-NMR (CDCl₃)δ: 3.91 (3H, s), 5.18 (2H, s), 6.56-6.66 (2H, m), 7.28-7.49 (6H, m), 10.59 (1H, s).

### Reference Example 128

To a solution of 2-benzyloxy-6-methoxybenzaldehyde (10.44 g) in tetrahydrofuran (100 ml) was added lithium aluminum hydride (1.23 g) at 0° C., and the mixture was stirred at room temperature for 1 hour. Sodium sulfate 10 hydrate (12.02 g) was added to the reaction mixture, and the mixture was stirred at room temperature for 1 hour. The precipitate was removed by filtration and the filtrate was concentrated. The residue was subjected to silica gel column chromatography, and 2-benzyloxy-6-methoxybenzyl alcohol (10.21 g, yield 97%) was obtained as a colorless oil from a fraction eluted with ethyl acetate.

¹H-NMR (CDCl₃)δ: 2.50 (1H, t, J=6.6 Hz), 3.86 (3H, s), 4.85 (2H, d, J=6.6 Hz), 5.11 (2H, s), 6.54-6.66 (2H, m), 7.14-7.48 (6H, m).

### Reference Example 129

To a mixture of 2-benzyloxy-6-methoxybenzyl alcohol (12.53 g), acetone cyanohydrin (7.27 g), triphenylphosphine (27.32 g) and tetrahydrofuran (250 ml) was dropwise added a 40% toluene solution (44.65 g) of diethyl azodicarboxylate at room temperature, and the mixture was stirred overnight. The reaction solution was concentrated. The residue was subjected to silica gel column chromatography, and (2-benzyloxy-6-methoxyphenyl)acetonitrile (11.46 g, yield 88%) was obtained as a colorless oil from a fraction eluted with ethyl acetate-hexane (1:4, volume ratio).

¹H-NMR (CDCl₃)δ: 3.73 (2H, s), 3.88 (3H, s), 5.13 (2H, s), 6.52-6.66 (2H, m), 7.17-7.50 (6H, m).

### Reference Example 130

A mixture of (2-benzyloxy-6-methoxyphenyl)acetonitrile (11.46 g), 8N aqueous sodium hydroxide solution (40 ml) and ethanol (200 ml) was stirred under reflux overnight. After cooling, the reaction mixture was acidified by slowly adding conc. hydrochloric acid (30 ml). After concentration, the residue was dissolved in ethyl acetate. The obtained ethyl acetate solution was washed with saturated aqueous sodium chloride solution, dried (MgSO₄) and concentrated. A mixture of the residue, a 10% solution (200 ml) of hydrochloric acid in methanol and methanol (200 ml) was stirred overnight at room temperature. After concentration, the residue was dissolved in ethyl acetate. The obtained ethyl acetate solution was washed with saturated aqueous sodium chloride solution, dried (MgSO₄) and concentrated. The residue was subjected to silica gel column chromatography, and methyl (2-benzyloxy-6-methoxyphenyl)acetate (6.43 g, yield 50%) was obtained as a colorless oil from a fraction eluted with ethyl acetate-hexane (1:4, volume ratio).

¹H-NMR (CDCl₃)δ: 3.63 (3H, s), 3.76 (2H, s), 3.82 (3H, s), 5.08 (2H, s), 6.52-6.64 (2H, m), 7.12-7.40 (6H, m).

### Reference Example 131

A mixture of methyl (2-benzyloxy-6-methoxyphenyl)acetate (6.43 g), 5% palladium-carbon (1.59 g) and tetrahydrofuran (100 ml) was stirred overnight at room temperature under a hydrogen atmosphere. Palladium-carbon was removed by filtration and the filtrate was concentrated. The residue was subjected to silica gel column chromatography, and methyl (6-hydroxy-2-methoxyphenyl)acetate (4.20 g, yield 95%) was obtained as a colorless oil from a fraction eluted with ethyl acetate-hexane (1:4, volume ratio).

¹H-NMR (CDCl₃)δ: 3.73 (3H, s), 3.77 (2H, s), 3.81 (3H, s), 6.40-6.62 (2H, m), 6.94 (1H, s), 7.06-7.18 (1H, m).

### Reference Example 132

To a mixture of 2-benzyloxy-6-methoxybenzaldehyde (3.30 g), ethyl diethylphosphonoacetate (3.60 g) and N,N-dimethylformamide (50 ml) was added sodium hydride (60%, in oil, 0.61 g) at 0° C., and the mixture was stirred overnight at room temperature. The reaction mixture was poured into water, and extracted with ethyl acetate. The ethyl acetate layer was washed with dilute hydrochloric acid and then with saturated aqueous sodium chloride solution, dried (MgSO₄) and concentrated. The residue was subjected to silica gel column chromatography to give ethyl (E)-3-(2-benzyloxy-6-methoxyphenyl)propenoate (3.86 g, yield 91%) as a colorless oil from a fraction eluted with ethyl acetate-hexane (1:4, volume ratio).

¹H-NMR (CDCl₃)δ: 1.32 (3H, t, J=7.0 Hz), 3.89 (3H, s), 4.24 (2H, q, J=7.0 Hz), 5.18 (2H, s), 6.53-6.62 (2H, m), 6.91 (1H, d, J=16.2 Hz), 7.16-7.47 (6H, m), 8.20 (1H, d, J=16.2 Hz).

### Reference Example 133

A mixture of ethyl (E)-3-(2-benzyloxy-6-methoxyphenyl)propenoate (3.86 g), 5% palladium-carbon (1.00 g) and tetrahydrofuran (50 ml), and the reaction mixture was poured into saturated aqueous ammonium chloride solution. Palladium-carbon was removed by filtration and the filtrate was concentrated. The residue was subjected to silica gel column chromatography, and ethyl 3-(6-hydroxy-2-methoxyphenyl)propionate (2.52 g, yield 90%) was obtained as a colorless oil from a fraction eluted with ethyl acetate-hexane (1:4, volume ratio).

¹H-NMR (CDCl₃)δ: 1.22 (3H, t, J=7.0 Hz), 2.65-2.75 (2H, m) 2.83-2.93 (2H, m), 3.80 (3H, s), 4.13 (2H, q, J=7.0 Hz), 6.45 (1H, d, J=8.0 Hz), 6.60 (1H, d, J=8.0 Hz), 7.02-7.14 (1H, m), 7.86 (1H, s).

### Reference Example 134

To a solution of ethyl 3-[1-(5-chloro-2-pyridyl)-3-(1-ethylpropyl)-1H-pyrazol-4-yl]propionate (3.92 g) in tetrahydrofuran (25 ml) was dropwise added a 1.0 M solution (25 ml) of diisobutylaluminum hydride in hexane at 0° C., and the mixture was stirred at room temperature for 1 hour. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and 3-[1-(5-chloro-2-pyridyl)-3-(1-ethylpropyl)-1H-pyrazol-4-yl]-1-propanol (3.15 g, yield 91%) was obtained as colorless crystals from a fraction eluted with ethyl acetate-hexane (1:1, volume ratio). The crystals were recrystallized from ethyl acetate-hexane. melting point: 62-63° C.

### Reference Example 135

To a solution of 3-benzyloxy-4-ethoxybenzaldehyde (5.34 g) in tetrahydrofuran (50 ml) was added lithium aluminum hydride (0.40 g) at 0° C., and the mixture was stirred at room temperature for 1 hour. Sodium sulfate 10 hydrate (4.02 g) was added to the reaction mixture, and the mixture was stirred at room temperature for 1 hour. The precipitate was removed by filtration and the filtrate was concentrated. The residue was subjected to silica gel column chromatography, and 3-benzyloxy-4-ethoxybenzyl alcohol (4.88 g, yield 91%) was obtained as a colorless oil from a fraction eluted with ethyl acetate.

¹H-NMR (CDCl₃)δ: 1.47 (3H, t, J=7.0 Hz), 4.13 (2H, q, J=7.0 Hz), 4.60 (2H, d, J=5.8 Hz), 5.14 (2H, s), 6.78-6.99 (3H, m), 7.26-7.50 (5H, m).

### Reference Example 136

A mixture of ethyl 3-oxoheptanate (10.16 g) and N,N-dimethylformamide dimethyl acetal (9.53 g) were refluxed for 1 hour, and concentrated under reduced pressure. The residue was dissolved in ethanol (250 ml) and a solution of hydrazine monohydrate (3.06 g) in ethanol (50 ml) was slowly added at room temperature, which was followed by stirring overnight. The reaction mixture was concentrated under reduced pressure and the residue was dissolved in ethyl acetate. The obtained ethyl acetate solution was washed with saturated aqueous sodium chloride solution, dried (MgSO₄) and concentrated. A mixture of the residue, 2-chloro-5-(trifluoromethyl)pyridine (11.35 g), potassium carbonate (13.00 g) and N,N-dimethylformamide (200 ml) was stirred overnight at 100° C. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and ethyl 3-butyl-1-[5-(trifluoromethyl)-2-pyridyl]-1H-pyrazole-4-carboxylate (17.25 g, yield 86%) was obtained as colorless crystals from a fraction eluted with ethyl acetate-hexane (1:4, volume ratio). The crystals were recrystallized from ethyl acetate-hexane. melting point: 58-59° C.

### Reference Example 137

To a solution of ethyl 3-butyl-1-[5-(trifluoromethyl)-2-pyridyl]-1H-pyrazole-4-carboxylate (16.50 g) in tetrahydrofuran (100 ml) was dropwise added a 1.0 M solution (100 ml) of diisobutylaluminum hydride in hexane at 0° C., and the mixture was stirred at room temperature for 1 hour. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and {3-butyl-1-[5-(trifluoromethyl)-2-pyridyl]-1H-pyrazol-4-yl}methanol (13.59 g, yield 94%) was obtained as colorless crystals from a fraction eluted with ethyl acetate-hexane (1:1, volume ratio). The crystals were recrystallized from ethyl acetate-hexane. melting point: 110-111° C.

### Reference Example 138

A mixture of {3-butyl-1-[5-(trifluoromethyl)-2-pyridyl]-1H-pyrazol-4-yl}methanol (6.00 g), activated manganese dioxide (18.19 g) and tetrahydrofuran (100 ml) was stirred overnight at room temperature. The insoluble material was removed by filtration and the filtrate was concentrated. The residue was subjected to silica gel column chromatography, and 3-butyl-1-[5-(trifluoromethyl)-2-pyridyl]-1H-pyrazole-4-carbaldehyde (5.16 g, yield 87%) was obtained as a colorless oil from a fraction eluted with ethyl acetate-hexane (1:4, volume ratio).

¹H-NMR (CDCl₃)δ: 0.97 (3H, t, J=7.4 Hz), 1.34-1.82 (4H m), 2.90-3.04 (2H, m), 8.03-8.17 (2H, m), 8.68-8.73 (1H, m), 9.03 (1H, s), 10.05 (1H, s).

### Reference Example 139

To a mixture of 3-butyl-1-[5-(trifluoromethyl)-2-pyridyl]-1H-pyrazole-4-carbaldehyde (4.33 g), ethyl diethylphosphonoacetate (3.95 g) and N,N-dimethylformamide (50 ml) was added, sodium hydride (60%, in oil, 0.64 g) at 0° C., and the mixture was stirred overnight at room temperature. The reaction mixture was poured into water, and extracted with ethyl acetate. The ethyl acetate layer was washed with dilute hydrochloric acid and then with saturated aqueous sodium chloride solution, dried (MgSO₄) and concentrated. The residue was subjected to silica gel column chromatography, and ethyl (E)-3-{3-butyl-1-[5-(trifluoromethyl)-2-pyridyl]-1H-pyrazol-4-yl}propenoate (4.81 g, yield 90%) was obtained as colorless crystals from a fraction eluted with ethyl acetate-hexane (1:4, volume ratio). The crystals were recrystallized from ethyl acetate-hexane. melting point: 84-85° C.

### Reference Example 140

A mixture of ethyl (E)-3-{3-butyl-1-[5-(trifluoromethyl)-2-pyridyl]-1H-pyrazol-4-yl}propenoate (3.50 g), 5% palladium-carbon (0.73 g) and tetrahydrofuran (50 ml) was stirred at room temperature for 1 hour under a hydrogen atmosphere. Palladium-carbon was removed by filtration and the filtrate was concentrated. The residue was subjected to silica gel column chromatography, and ethyl 3-{3-butyl-1-[5-(trifluoromethyl)-2-pyridyl]-1H-pyrazol-4-yl}propionate (3.31 g, yield 94%) was obtained as colorless crystals from a fraction eluted with ethyl acetate-hexane (1:2, volume ratio). The crystals were recrystallized from ethyl acetate-hexane. melting point: 63-64° C.

### Reference Example 141

To a solution of ethyl 3-{3-butyl-1-[5-(trifluoromethyl)-2-pyridyl]-1H-pyrazol-4-yl}propionate (3.00 g) in tetrahydrofuran (50 ml) was dropwise added a 1.0 M solution (20 ml) of diisobutylaluminum hydride in hexane at 0° C., and the mixture was stirred at room temperature for 1 hour. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and 3-{3-butyl-1-[5-(trifluoromethyl)-2-pyridyl]-1H-pyrazol-4-yl}-1-propanol (2.43 g, yield 91%) was obtained as colorless crystals from a fraction eluted with ethyl acetate-hexane (1:1, volume ratio). The crystals were recrystallized from ethyl acetate-hexane. melting point: 93-94° C.

### Reference Example 142

To a solution of ethyl 3-[3-(1-ethylpropyl)-1H-pyrazol-4-yl]propionate (3.30 g) in N,N-dimethylformamide (40 ml) was added sodium hydride (60%, in oil, 0.57 g) at 0° C. and the mixture was stirred at room temperature for 15 minutes. 2,5-Dichloropyridine (2.10 g) was added at room temperature, and stirred overnight at 100° C. The reaction mixture was poured into water, and extracted with ethyl acetate. The ethyl acetate layer was washed with saturated aqueous sodium chloride solution, dried (MgSO₄) and concentrated. The residue was subjected to silica gel column chromatography, and ethyl 3-[1-(5-chloro-2-pyridyl)-3-(1-ethylpropyl)-1H-pyrazol-4-yl]propionate (3.92 g, yield 81%) was obtained as a colorless oil from a fraction eluted with ethyl acetate-hexane (1:4, volume ratio).

¹H-NMR (CDCl₃)δ: 0.86 (6H, t, J=7.2 Hz), 1.26 (3H, t, J=7.2 Hz), 1.60-1.86 (4H, m), 2.48-2.88 (5H, m), 4.16 (2H, q, J=7.2 Hz), 7.69 (1H, d, J=2.6, 8.8 Hz), 7.84-7.92 (1H, m), 8.20 (1H, s), 8.26-8.39 (1H, m).

### Reference Example 143

A mixture of ethyl 3-(3-propyl-1H-pyrazol-4-yl)propanoate (1.30 g), 4-(trifluoromethyl)phenylboric acid (2.37 g), copper(II) acetate (1.69 g), pyridine (0.9 ml) and N,N-dimethylformamide (50 ml) was stirred overnight at room temperature. The precipitate was removed by filtration and the filtrate was concentrated. The residue was subjected to silica gel column chromatography, and a colorless oil was obtained from a fraction eluted with ethyl acetate-hexane (1:9, volume ratio). To a solution of the obtained colorless oil in tetrahydrofuran (30 ml) was added lithium aluminum hydride (0.23 g) at 0° C., and the mixture was stirred at room temperature for 1 hour. The sodium sulfate 10 hydrate (2.10 g) was added to the reaction mixture, and the mixture was stirred at room temperature for 1 hour. The precipitate was removed by filtration and the filtrate was concentrated. The residue was subjected to silica gel column chromatography, and 3-{3-propyl-1-[4-(trifluoromethyl)phenyl]-1H-pyrazol-4-yl}-1-propanol (0.87 g, yield 45%) was obtained as a colorless oil from a fraction eluted with ethyl acetate-hexane (1:2, volume ratio).

¹H-NMR (CDCl₃)δ: 1.02 (3H, t, J=7.0 Hz), 1.36 (1H, br t), 1.64-1.98 (4H, m), 2.52-2.69 (4H, m), 3.68-3.81 (2H, m), 7.60-7.80 (5H, m).

### Reference Example 144

A mixture of ethyl 3-isopropyl-1H-pyrazole-4-carboxylate (5.00 g), 4-(trifluoromethyl)phenylboric acid (10.45 g), copper(II) acetate (7.50 g), pyridine (4.0 ml) and N,N-dimethylformamide (75 ml) was stirred overnight at room temperature. The precipitate was removed by filtration, and the filtrate was concentrated. The residue was subjected to silica gel column chromatography, and ethyl 3-isopropyl-1-[4-(trifluoromethyl)phenyl]-1H-pyrazole-4-carboxylate (6.93 g, yield 77%) was obtained as colorless crystals from a fraction eluted with ethyl acetate-hexane (1:4, volume ratio). The crystals were recrystallized from ethyl acetate-hexane. melting point: 74-75° C.

### Reference Example 145

To a solution of ethyl 3-isopropyl-1-[4-(trifluoromethyl)phenyl]-1H-pyrazole-4-carboxylate (6.00 g) in tetrahydrofuran (30 ml) was added lithium aluminum hydride (0.54 g) at 0° C., and the mixture was stirred at room temperature for 1 hour. Sodium sulfate 10 hydrate (5.10 g) was added to the reaction mixture, and the mixture was stirred at room temperature for 1 hour. The precipitate was removed by filtration and the filtrate was concentrated. The residue was subjected to silica gel column chromatography, and {3-isopropyl-1-[4-(trifluoromethyl)phenyl]-1H-pyrazol-4-yl}methanol (4.86 g, yield 93%) was obtained as colorless crystals from a fraction eluted with ethyl acetate-hexane (1:2, volume ratio). The crystals were recrystallized from ethyl acetate-hexane. melting point: 84-85° C.

### Reference Example 146

A mixture of {3-isopropyl-1-[4-(trifluoromethyl)phenyl]-1H-pyrazol-4-yl}methanol (2.35 g), activated manganese dioxide (7.90 g) and tetrahydrofuran (50 ml) was stirred overnight at room temperature. The insoluble material was removed by filtration and the filtrate was concentrated. The residue was subjected to silica gel column chromatography, and 3-isopropyl-1-[4-(trifluoromethyl)phenyl]-1H-pyrazole-4-carbaldehyde (2.25 g, yield 96%) was obtained as colorless crystals from a fraction eluted with ethyl acetate-hexane (1:4, volume ratio). The crystals were recrystallized from ethyl acetate-hexane. melting point: 81-82° C.

### Reference Example 147

To a mixture of 3-isopropyl-1-[4-(trifluoromethyl)phenyl]-1H-pyrazole-4-carbaldehyde (2.10 g), ethyl diethylphosphonoacetate (2.50 g) and N,N-dimethylformamide (30 ml) was added sodium hydride (60%, in oil, 0.36 g) at 0° C., and the mixture was stirred overnight at room temperature. The reaction mixture was poured into water, and extracted with ethyl acetate. The ethyl acetate layer was washed with dilute hydrochloric acid and then with saturated aqueous sodium chloride solution, dried (MgSO₄) and concentrated. The residue was subjected to silica gel column chromatography, and ethyl (E)-3-{3-isopropyl-1-[4-(trifluoromethyl)phenyl]-1H-pyrazol-4-yl}propenoate (2.47 g, yield 94%) was obtained as colorless crystals from a fraction eluted with ethyl acetate-hexane (1:4, volume ratio). The crystals were recrystallized from ethyl acetate-hexane. melting point: 118-119° C.

### Reference Example 148

A mixture of ethyl (E)-3-{3-isopropyl-1-[4-(trifluoromethyl)phenyl]-1H-pyrazol-4-yl}propenoate (2.30 g), 5% palladium-carbon (0.82 g) and tetrahydrofuran (50 ml) was stirred at room temperature for 1 hour under a hydrogen atmosphere. Palladium-carbon was removed by filtration and the filtrate was concentrated. The residue was subjected to silica gel column chromatography, and ethyl 3-{3-isopropyl-1-[4-(trifluoromethyl)phenyl]-1H-pyrazol-4-yl}propionate (2.30 g, 99%) was obtained as a colorless oil from a fraction eluted with ethyl acetate-hexane (1:2, volume ratio).

¹H-NMR (CDCl₃)δ: 1.26 (3H, t, J=7.0 Hz), 1.34 (6H, d, J=6.8 Hz), 2.56-2.67 (2H, m), 2.79-2.90 (2H, m), 2.96-3.13 (1H, m), 4.16 (2H, q, J=7.0 Hz), 7.61-7.80 (5H, m).

### Reference Example 149

To a solution of ethyl 3-{3-isopropyl-1-[4-(trifluoromethyl)phenyl]-1H-pyrazol-4-yl}propionate (2.30 g) in tetrahydrofuran (20 ml) was added lithium aluminum hydride (0.25 g) at 0° C., and the mixture was stirred at room temperature for 1 hour. Sodium sulfate 10 hydrate (2.30 g) was added to the reaction mixture, and the mixture was stirred at room temperature for 1 hour. The precipitate was removed by filtration and the filtrate was concentrated. The residue was subjected to silica gel column chromatography, and 3-{3-isopropyl-1-[4-(trifluoromethyl)phenyl]-1H-pyrazol-4-yl}-1-propanol (1.89 g, yield 93%) was obtained as a colorless oil from a fraction eluted with ethyl acetate-hexane (1:2, volume ratio).

¹H-NMR (CDCl₃)δ: 1.34 (6H, d, J=6.8 Hz), 1.80-1.98 (2H, m) 2.53-2.67 (2H, m), 2.94-3.13 (1H, m), 3.68-3.82 (2H, m), 7.61-7.80 (5H, m).

### Reference Example 150

A mixture of ethyl 3-cyclohexyl-3-oxopropionate (12.60 g) and N,N-dimethylformamide dimethyl acetal (11.33 g) was refluxed for 1 hour, and concentrated under reduced pressure. The residue was dissolved in ethanol (150 ml) and a solution of hydrazine monohydrate (3.20 g) in ethanol (150 ml) was slowly added at room temperature. The mixture was stirred overnight. The reaction mixture was concentrated under reduced pressure and the residue was dissolved in ethyl acetate. The obtained ethyl acetate solution was washed with saturated aqueous sodium chloride solution, dried (MgSO₄) and concentrated. A mixture of the residue, 2-chloro-5-(trifluoromethyl)pyridine (12.06 g), potassium carbonate (15.94 g) and N,N-dimethylformamide (200 ml) was stirred overnight at 100° C. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and ethyl 3-cyclohexyl-1-[5-(trifluoromethyl)-2-pyridyl]-1H-pyrazole-4-carboxylate (20.15 g, yield 86%) was obtained as colorless crystals from a fraction eluted with ethyl acetate-hexane (1:4, volume ratio). The crystals were recrystallized from ethyl acetate-hexane. melting point: 99-100° C.

### Reference Example 151

To a solution of ethyl 3-cyclohexyl-1-[5-(trifluoromethyl)-2-pyridyl]-1H-pyrazole-4-carboxylate (20.00 g) in tetrahydrofuran (150 ml) was dropwise added a 1.0 M solution (120 ml) of diisobutylaluminum hydride in hexane at 0° C., and the mixture was stirred at room temperature for 1 hour. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and {3-cyclohexyl-1-[5-(trifluoromethyl)-2-pyridyl]-1H-pyrazol-4-yl}methanol (16.39 g, yield 93%) was obtained as colorless crystals from a fraction eluted with ethyl acetate-hexane (1:1, volume ratio). The crystals were recrystallized from ethyl acetate-hexane. melting point: 138-139° C.

### Reference Example 152

A mixture of {3-cyclohexyl-1-[5-(trifluoromethyl)-2-pyridyl]-1H-pyrazol-4-yl}methanol (7.10 g), activated manganese dioxide (22.90 g) and tetrahydrofuran (100 ml) was stirred overnight at room temperature. The insoluble material was removed by filtration and the filtrate was concentrated. The residue was subjected to silica gel column chromatography, and 3-cyclohexyl-1-[5-(trifluoromethyl)-2-pyridyl]-1H-pyrazole-4-carbaldehyde (6.69 g, yield 95%) was obtained as colorless crystals from a fraction eluted with ethyl acetate-hexane (1:4, volume ratio). The crystals were recrystallized from ethyl acetate-hexane. melting point: 103-104° C.

### Reference Example 153

To a mixture of 3-cyclohexyl-1-[5-(trifluoromethyl)-2-pyridyl]-1H-pyrazole-4-carbaldehyde (6.40 g), ethyl diethylphosphonoacetate (5.33 g) and N,N-dimethylformamide (50 ml) was added sodium hydride (60%, in oil, 0.93 g) at 0° C., and the mixture was stirred overnight at room temperature. The reaction mixture was poured into water, and extracted with ethyl acetate. The ethyl acetate layer was washed with dilute hydrochloric acid and then with saturated aqueous sodium chloride solution, dried (MgSO₄) and concentrated. The residue was subjected to silica gel column chromatography, and ethyl (E)-3-{3-cyclohexyl-1-[5-(trifluoromethyl)-2-pyridyl]-1H-pyrazol-4-yl}propenoate (7.53 g, yield 96%) was obtained as colorless crystals from a fraction eluted with ethyl acetate-hexane (1:4, volume ratio). The crystals were recrystallized from ethyl acetate-hexane. melting point: 132-133° C.

### Reference Example 154

A mixture of ethyl (E)-3-{3-cyclohexyl-1-[5-(trifluoromethyl)-2-pyridyl]-1H-pyrazol-4-yl}propenoate (7.40 g), 5% palladium-carbon (1.49 g) and tetrahydrofuran (100 ml) was stirred at room temperature for 1 hour under a hydrogen atmosphere. Palladium-carbon was removed by filtration and the filtrate was concentrated. The residue was subjected to silica gel column chromatography, and ethyl 3-{3-cyclohexyl-1-[5-(trifluoromethyl)-2-pyridyl]-1H-pyrazol-4-yl}propionate (7.20 g, yield 97%) was obtained as a colorless oil from a fraction eluted with ethyl acetate-hexane (1:2, volume ratio).

¹H-NMR (CDCl₃)δ: 1.32-2.00 (13H, m), 2.58-2.88 (5H, m), 4.16 (2H, q, J=7.0 Hz), 7.89-8.05 (2H, m), 8.27 (1H, s), 8.56-8.64 (1H, m).

### Reference Example 155

To a solution of ethyl 3-{3-cyclohexyl-1-[5-(trifluoromethyl)-2-pyridyl]-1H-pyrazol-4-yl}propionate (7.20 g) in tetrahydrofuran (60 ml) was dropwise added a 1.0 M solution (40 ml) of diisobutylaluminum hydride in hexane at 0° C., and the mixture was stirred at room temperature for 1 hour. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and 3-{3-cyclohexyl-1-[5-(trifluoromethyl)-2-pyridyl]-1H-pyrazol-4-yl}-1-propanol (5.83 g, yield 91%) was obtained as colorless crystals from a fraction eluted with ethyl acetate-hexane (1:1, volume ratio). The crystals were recrystallized from ethyl acetate-hexane. melting point: 106-107° C.

### Reference Example 156

To a mixture of 3-benzyloxy-4-ethoxybenzyl alcohol (4.80 g), acetone cyanohydrin (3.50 g), triphenylphosphine (9.86 g) and tetrahydrofuran (100 ml) was dropwise added a 40% toluene solution (16.16 g) of diethyl azodicarboxylate at room temperature, and the mixture was stirred overnight. The reaction solution was concentrated. The residue was subjected to silica gel column chromatography, and (3-benzyloxy-4-ethoxyphenyl)acetonitrile (3.68 g, yield 74%) was obtained as a colorless oil from a fraction eluted with ethyl acetate-hexane (1:4, volume ratio).

¹H-NMR (CDCl₃)δ: 1.47 (3H, t, J=6.8 Hz), 3.67 (2H, s), 4.12 (2H, q, J=6.8 Hz), 5.15 (2H, s), 6.74-6.96 (3H, m), 7.28-7.47 (5H, m).

### Reference Example 157

A mixture of (3-benzyloxy-4-ethoxyphenyl)acetonitrile (3.68 g), 4N aqueous sodium hydroxide solution (10 ml) and ethanol (50 ml) was stirred under reflux overnight. After cooling, the reaction mixture was acidified by slowly adding conc. hydrochloric acid (5 ml). After concentration, the residue was dissolved in ethyl acetate. The obtained ethyl acetate solution was washed with saturated aqueous sodium chloride solution, dried (MgSO₄) and concentrated. A mixture of the residue, a 10% solution (20 ml) of hydrochloric acid in methanol and methanol (50 ml) was stirred overnight at room temperature. After concentration, the residue was dissolved in ethyl acetate. The obtained ethyl acetate solution was washed with saturated aqueous sodium chloride solution, dried (MgSO₄) and concentrated. The residue was subjected to silica gel column chromatography, and methyl (3-benzyloxy-4-ethoxyphenyl)acetate (2.99 g, yield 72%) was obtained as a colorless oil from a fraction eluted with ethyl acetate-hexane (1:4, volume ratio).

¹H-NMR (CDCl₃)δ: 1.45 (3H, t, J=7.0 Hz), 3.54 (2H, s), 3.69 (3H, s), 4.11 (2H, q, J=7.0 Hz), 5.13 (2H, s), 6.70-6.88 (3H, m), 7.27-7.48 (5H, m).

### Reference Example 158

A mixture of methyl (3-benzyloxy-4-ethoxyphenyl)acetate (2.99 g), 5% palladium-carbon (0.61 g) and tetrahydrofuran (50 ml) was stirred overnight at room temperature under a hydrogen atmosphere. Palladium-carbon was removed by filtration and the filtrate was concentrated. The residue was subjected to silica gel column chromatography, and methyl (4-ethoxy-3-hydroxyphenyl)acetate (1.89 g, yield 90%) was obtained as a colorless oil from a fraction eluted with ethyl acetate-hexane (1:4, volume ratio).

¹H-NMR (CDCl₃)δ: 1.44 (3H, t, J=7.0 Hz), 3.54 (2H, s), 3.69 (3H, s), 4.11 (2H, q, J=7.0 Hz), 5.61 (1H, s), 6.72-6.89 (3H, m).

### Reference Example 159

A mixture of 3-fluorosalicylaldehyde (5.20 g), benzyl bromide (4.5 ml), potassium carbonate (5.26 g) and N,N-dimethylformamide (75 ml) was stirred overnight at room temperature. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and 2-benzyloxy-3-fluorobenzaldehyde (8.24 g, yield 96%) was obtained as a colorless oil from a fraction eluted with ethyl acetate-hexane (1:4, volume ratio).

¹H-NMR (CDCl₃)δ: 5.28 (2H, s), 7.07-7.16 (1H, m), 7.24-7.42 (6H, m), 7.56-7.60 (1H, m), 10.25 (1H, s).

### Reference Example 160

To a solution of 2-benzyloxy-3-fluorobenzaldehyde (8.24 g) in tetrahydrofuran (50 ml) was added lithium aluminum hydride (0.45 g) at 0° C., and the mixture was stirred at room temperature for 1 hour. Sodium sulfate 10 hydrate (4.02 g) was added to the reaction mixture, and the mixture was stirred at room temperature for 1 hour. The precipitate was removed by filtration and the filtrate was concentrated. The residue was subjected to silica gel column chromatography, and 2-benzyloxy-3-fluorobenzyl alcohol (8.18 g, yield 98%) was obtained as a colorless oil from a fraction eluted with ethyl acetate.

¹H-NMR (CDCl₃)δ: 1.87 (1H, t, J=6.6 Hz), 4.58 (2H, d, J=6.6 Hz), 5.17 (2H, s), 6.97-7.13 (3H, m), 7.34-7.46 (5H, m).

### Reference Example 161

To a mixture of 2-benzyloxy-3-fluorobenzyl alcohol (8.10 g), acetone cyanohydrin (4.95 g), triphenylphosphine (18.57 g) and tetrahydrofuran (150 ml) was dropwise added a 40% solution (30.36 g) of diethyl azodicarboxylate in toluene at room temperature, and the mixture was stirred overnight. The reaction solution was concentrated. The residue was subjected to silica gel column chromatography, and 2-benzyloxy-3-fluorophenylacetonitrile (7.20 g, yield 85%) was obtained as a colorless oil from a fraction eluted with ethyl acetate-hexane (1:4, volume ratio).

¹H-NMR (CDCl₃)δ: 3.56 (2H, s), 5.19 (2H, s), 6.98-7.18 (3H, m), 7.30-7.46 (5H, m).

### Reference Example 162

A mixture of 2-benzyloxy-3-fluorophenylacetonitrile (7.20 g), 4N aqueous sodium hydroxide solution (10 ml) and ethanol (50 ml) was stirred under reflux overnight. After cooling, the reaction mixture was acidified by slowly adding conc. hydrochloric acid (4 ml). After concentration, the residue was dissolved in ethyl acetate. The obtained ethyl acetate solution was washed with saturated aqueous sodium chloride solution, dried (MgSO₄) and concentrated. A mixture of the residue, a 10% solution (50 ml) of hydrochloric acid in methanol and methanol (50 ml) was stirred overnight at room temperature. After concentration, the residue was dissolved in ethyl acetate. The obtained ethyl acetate solution was washed with saturated aqueous sodium chloride solution, dried (MgSO₄) and concentrated. The residue was subjected to silica gel column chromatography, and methyl (2-benzyloxy-3-fluorophenyl)acetate (6.63 g, yield 81%) was obtained as a colorless oil from a fraction eluted with ethyl acetate-hexane (1:4, volume ratio).

¹H-NMR (CDCl₃)δ: 3.62 (5H, s), 5.12 (2H, s), 6.94-7.12 (3H, m), 7.26-7.47 (5H, m).

### Reference Example 163

A mixture of methyl (2-benzyloxy-3-fluorophenyl)acetate (6.63 g), 5% palladium-carbon (1.44 g) and tetrahydrofuran (150 ml) was stirred overnight at room temperature under a hydrogen atmosphere. Palladium-carbon was removed by filtration and the filtrate was concentrated. The residue was subjected to silica gel column chromatography, and methyl (3-fluoro-2-hydroxyphenyl)acetate (4.53 g, yield 98%) was obtained as a colorless oil from a fraction eluted with ethyl acetate-hexane (1:4, volume ratio).

¹H-NMR (CDCl₃)δ: 1.58 (1H, br t), 3.71 (2H, s), 3.74 (3H, s), 6.74-7.08 (3H, m).

### Reference Example 164

A mixture of [1-(5-chloro-2-pyridyl)-3-isopropyl-1H-pyrazol-4-yl]methanol (2.00 g), activated manganese dioxide (6.08 g) and tetrahydrofuran (50 ml) was stirred overnight at room temperature. The insoluble material was removed by filtration and the filtrate was concentrated. The residue was subjected to silica gel column chromatography, and 1-(5-chloro-2-pyridyl)-3-isopropyl-1H-pyrazole-4-carbaldehyde (1.84 g, yield 93%) was obtained as colorless crystals from a fraction eluted with ethyl acetate-hexane (1:4, volume ratio). The crystals were recrystallized from ethyl acetate-hexane. melting point: 69-70° C.

### Reference Example 165

To a mixture of 1-(5-chloro-2-pyridyl)-3-isopropyl-1H-pyrazole-4-carbaldehyde (1.50 g), ethyl diethylphosphonoacetate (1.62 g) and N,N-dimethylformamide (30 ml) was added sodium hydride (60%, in oil, 0.27 g) at 0° C. and the mixture was stirred overnight at room temperature. The reaction mixture was poured into water, and extracted with ethyl acetate. The ethyl acetate layer was washed with dilute hydrochloric acid and then with saturated aqueous sodium chloride solution, dried (MgSO₄) and concentrated. The residue was subjected to silica gel column chromatography, and ethyl (E)-3-[1-(5-chloro-2-pyridyl)-3-isopropyl-1H-pyrazol-4-yl]propenoate (1.83 g, yield 95%) was obtained as colorless crystals from a fraction eluted with ethyl acetate-hexane (1:4, volume ratio). The crystals were recrystallized from ethyl acetate-hexane. melting point: 105-106° C.

### Reference Example 166

A mixture of 2-ethylbutanoic acid (7.03 g), 1,1′-carbonyldiimidazole (10.30 g) and tetrahydrofuran (200 ml) was refluxed for 1.5 hours. After cooling to room temperature, magnesium chloride (6.66 g) and potassium ethyl malonate (11.90 g) were added and the mixture was refluxed for 1.5 hours. The reaction solution was acidified with dilute hydrochloric acid, and extracted with ethyl acetate. The ethyl acetate layer was washed with saturated aqueous sodium chloride solution, dried (MgSO₄) and concentrated. A mixture of the residue and N,N-dimethylformamide dimethyl acetal (15.00 g) was refluxed for 1 hour, and concentrated under reduced pressure. The residue was dissolved in ethanol (100 ml), and a solution of hydrazine monohydrate (3.03 g) in ethanol (30 ml) was slowly added at room temperature. The mixture was stirred overnight. The reaction mixture was concentrated under reduced pressure and the residue was dissolved in ethyl acetate. The obtained ethyl acetate solution was washed with saturated aqueous sodium chloride solution, dried (MgSO₄) and concentrated. The residue was subjected to silica gel column chromatography, and ethyl 3-(1-ethylpropyl)-1H-pyrazole-4-carboxylate (9.83 g, yield 77%) was obtained as a colorless oil from a fraction eluted with ethyl acetate-hexane (1:2, volume ratio).

¹H-NMR (CDCl₃)δ: 0.85 (6H, t, J=7.0 Hz), 1.36 (3H, t, J=7.0 Hz), 1.50-1.88 (4H, m), 3.28-3.50 (1H, m), 4.29 (2H, q, J=7.0 Hz), 7.96 (1H, s).

### Reference Example 167

A mixture of ethyl 3-(1-ethylpropyl)-1H-pyrazole-4-carboxylate (5.00 g), 2-chloro-5-(trifluoromethyl)pyridine (4.35 g), potassium carbonate (4.84 g) and N,N-dimethylformamide (75 ml) was stirred overnight at 100° C. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and ethyl 3-(1-ethylpropyl)-1-[5-(trifluoromethyl)-2-pyridyl]-1H-pyrazole-4-carboxylate (7.45 g, yield 88%) was obtained as a colorless oil from a fraction eluted with ethyl acetate-hexane (1:4, volume ratio).

¹H-NMR (CDCl₃)δ: 0.88 (6H, t, J=7.2 Hz), 1.38 (3H, t, J=7.0 Hz), 1.60-1.95 (4H, m), 3.20-3.40 (1H, m), 4.32 (2H, q, J=7.0 Hz), 7.98-8.17 (2H, m), 8.65-8.70 (1H, m), 8.99 (1H, s).

### Reference Example 168

To a solution of ethyl 3-(1-ethylpropyl)-1-[5-(trifluoromethyl)-2-pyridyl]-1H-pyrazole-4-carboxylate (6.58 g) in tetrahydrofuran (50 ml) was dropwise added a 1.0 M solution (40 ml) of diisobutylaluminum hydride in hexane at 0° C., and the mixture was stirred at room temperature for 1 hour. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and {3-(1-ethylpropyl)-1-[5-(trifluoromethyl)-2-pyridyl]-1H-pyrazol-4-yl}methanol (5.16 g, yield 89%) was obtained as a colorless oil from a fraction eluted with ethyl acetate-hexane (1:1, volume ratio).

¹H-NMR (CDCl₃)δ: 0.88 (6H, t, J=7.4 Hz), 1.42 (1H, t, J=5.2 Hz), 1.66-1.88 (4H, m), 2.60-2.80 (1H, m), 4.64 (2H, d, J=5.2 Hz), 7.93-8.11 (2H, m), 8.50 (1H, s), 8.61-8.65 (1H, m).

### Reference Example 169

A mixture of {3-(1-ethylpropyl)-1-[5-(trifluoromethyl)-2-pyridyl]-1H-pyrazol-4-yl}methanol (5.00 g), activated manganese dioxide (15.18 g) and tetrahydrofuran (50 ml) was stirred overnight at room temperature. The insoluble material was removed by filtration and the filtrate was concentrated. The residue was subjected to silica gel column chromatography, and 3-(1-ethylpropyl)-1-[5-(trifluoromethyl)-2-pyridyl]-1H-pyrazole-4-carbaldehyde (4.75 g, yield 95%) was obtained as a colorless oil from a fraction eluted with ethyl acetate-hexane (1:4, volume ratio).

¹H-NMR (CDCl₃)δ: 0.88 (6H, t, J=7.4 Hz), 1.68-1.94 (4H, m), 3.08-3.20 (1H, m), 8.02-8.17 (2H, m), 8.67-8.72 (1H, m), 9.03 (1H, s), 10.03 (1H, s).

### Reference Example 170

To a mixture of 3-(1-ethylpropyl)-1-[5-(trifluoromethyl)-2-pyridyl]-1H-pyrazole-4-carbaldehyde (4.70 g), ethyl diethylphosphonoacetate (4.06 g) and N,N-dimethylformamide (50 ml) was added sodium hydride (60%, in oil, 0.66 g) at 0° C. and the mixture was stirred overnight at room temperature. The reaction mixture was poured into water, and extracted with ethyl acetate. The ethyl acetate layer was washed with dilute hydrochloric acid and then with saturated aqueous sodium chloride solution, dried (MgSO₄) and concentrated. The residue was subjected to silica gel column chromatography, and ethyl (E)-3-{3-(1-ethylpropyl)-1-[5-(trifluoromethyl)-2-pyridyl]-1H-pyrazol-4-yl}propenoate (5.45 g, yield 95%) was obtained as a colorless oil from a fraction eluted with ethyl acetate-hexane (1:4, volume ratio).

¹H-NMR (CDCl₃)δ: 0.88 (6H, t, J=7.4 Hz), 1.34 (3H, t, J=7.0 Hz), 1.66-1.90 (4H, m), 2.70-2.88 (1H, m), 4.26 (2H, q, J=7.0 Hz), 6.30 (1H, d, J=16.0 Hz), 7.61 (1H, d, J=16.0 Hz), 7.97-8.14 (2H, m), 8.62-8.69 (1H, m), 8.78 (1H, s).

### Reference Example 171

A mixture of ethyl (E)-3-{3-(1-ethylpropyl)-1-[5-(trifluoromethyl)-2-pyridyl]-1H-pyrazol-4-yl}propenoate (5.45 g), 5% palladium-carbon (1.02 g) and tetrahydrofuran (50 ml) was stirred at room temperature for 1 hour under a hydrogen atmosphere. Palladium-carbon was removed by filtration and the filtrate was concentrated. The residue was subjected to silica gel column chromatography, and ethyl 3-{3-(1-ethylpropyl)-1-[5-(trifluoromethyl)-2-pyridyl]-1H-pyrazol-4-yl}propionate (5.28 g, yield 97%) was obtained as a colorless oil from a fraction eluted with ethyl acetate-hexane (1:2, volume ratio).

¹H-NMR (CDCl₃)δ: 0.87 (6H, t, J=7.2 Hz), 1.27 (3H, t, J=7.0 Hz), 1.64-1.86 (4H, m), 2.51-2.68 (3H, m), 2.76-2.88 (2H, m), 4.16 (2H, q, J=7.0 Hz), 7.90-8.07 (2H, m), 8.29 (1H, s), 8.58-8.62 (1H, m).

### Reference Example 172

To a solution of ethyl 3-{3-(1-ethylpropyl)-1-[5-(trifluoromethyl)-2-pyridyl]-1H-pyrazol-4-yl}propionate (5.20 g) in tetrahydrofuran (30 ml) was dropwise added a 1.0 M solution (30 ml) of diisobutylaluminum hydride in hexane at 0° C., and the mixture was stirred at room temperature for 1 hour. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and 3-{3-(1-ethylpropyl)-1-[5-(trifluoromethyl)-2-pyridyl]-1H-pyrazol-4-yl}-1-propanol (4.29 g, yield 93%) was obtained as colorless crystals from a fraction eluted with ethyl acetate-hexane (1:1, volume ratio). The crystals were recrystallized from ethyl acetate-hexane. melting point: 79-80° C.

### Reference Example 173

A mixture of 2-methylbutanoic acid (10.27 g), 1,1′-carbonyldiimidazole (16.48 g) and tetrahydrofuran (200 ml) was refluxed for 1.5 hours. After cooling to room temperature, magnesium chloride (10.58 g) and potassium ethyl malonate (18.92 g) were added and the mixture was refluxed for 1.5 hours. The reaction solution was acidified with dilute hydrochloric acid, and extracted with ethyl acetate. The ethyl acetate layer was washed with saturated aqueous sodium chloride solution, dried (MgSO₄) and concentrated. A mixture of the residue and N,N-dimethylformamide dimethyl acetal (18.05 g) was refluxed for 1 hour, and concentrated under reduced pressure. The residue was dissolved in ethanol (150 ml) and a solution of hydrazine monohydrate (5.13 g) in ethanol (50 ml) was slowly added at room temperature. The mixture was stirred overnight. The reaction mixture was concentrated under reduced pressure and the residue was dissolved in ethyl acetate. The obtained ethyl acetate solution was washed with saturated aqueous sodium chloride solution, dried (MgSO₄) and concentrated. The residue was subjected to silica gel column chromatography, and ethyl 3-(1-methylpropyl)-1H-pyrazole-4-carboxylate (14.48 g, yield 73%) was obtained as a colorless oil from a fraction eluted with ethyl acetate-hexane (1:2, volume ratio).

¹H-NMR (CDCl₃)δ: 0.91 (3H, t, J=7.2 Hz), 1.31 (3H, d, J=7.2 Hz), 1.36 (3H, t, J=7.2 Hz), 1.50-1.82 (2H, m), 3.44-3.58 (1H, m), 4.29 (2H, q, J=7.2 Hz), 7.94 (1H, s).

### Reference Example 174

A mixture of ethyl 3-(1-methylpropyl)-1H-pyrazole-4-carboxylate (10.00 g), 2-chloro-5-(trifluoromethyl)pyridine (9.38 g), potassium carbonate (8.66 g) and N,N-dimethylformamide (100 ml) was stirred overnight at 100° C. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and ethyl 3-(1-methylpropyl)-1-[5-(trifluoromethyl)-2-pyridyl]-1H-pyrazole-4-carboxylate (15.39 g, yield 88%) was obtained as colorless crystals from a fraction eluted with ethyl acetate-hexane (1:4, volume ratio). The crystals were recrystallized from ethyl acetate-hexane. melting point: 63-64° C.

### Reference Example 175

To a solution of ethyl 3-(1-methylpropyl)-1-[5-(trifluoromethyl)-2-pyridyl]-1H-pyrazole-4-carboxylate (13.44 g) in tetrahydrofuran (100 ml) was dropwise added a 1.0 M solution (90 ml) of diisobutylaluminum hydride in hexane at 0° C., and the mixture was stirred at room temperature for 1 hour. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and {3-(1-methylpropyl)-1-[5-(trifluoromethyl)-2-pyridyl]-1H-pyrazol-4-yl}methanol (10.86 g, yield 92%) was obtained as colorless crystals from a fraction eluted with ethyl acetate-hexane (1:1, volume ratio). The crystals were recrystallized from ethyl acetate-hexane. melting point: 76-77° C.

### Reference Example 176

A mixture of {3-(1-methylpropyl)-1-[5-(trifluoromethyl)-2-pyridyl]-1H-pyrazol-4-yl}methanol (8.00 g), activated manganese dioxide (24.16 g) and tetrahydrofuran (100 ml) was stirred overnight at room temperature. The insoluble material was removed by filtration and the filtrate was concentrated. The residue was subjected to silica gel column chromatography, and 3-(1-methylpropyl)-1-[5-(trifluoromethyl)-2-pyridyl]-1H-pyrazole-4-carbaldehyde (7.39 g, yield 93%) was obtained as colorless crystals from a fraction eluted with ethyl acetate-hexane (1:4, volume ratio). The crystals were recrystallized from ethyl acetate-hexane. melting point: 82-83° C.

### Reference Example 177

To a mixture of 3-(1-methylpropyl)-1-[5-(trifluoromethyl)-2-pyridyl]-1H-pyrazole-4-carbaldehyde (6.50 g), ethyl diethylphosphonoacetate (5.06 g) and N,N-dimethylformamide (50 ml) was added sodium hydride (60%, in oil, 0.88 g) at 0° C. and the mixture was stirred overnight at room temperature. The reaction mixture was poured into water, and extracted with ethyl acetate. The ethyl acetate layer was washed with dilute hydrochloric acid and then with saturated aqueous sodium chloride solution, dried (MgSO₄) and concentrated. The residue was subjected to silica gel column chromatography, and ethyl (E)-3-{3-(1-methylpropyl)-1-[5-(trifluoromethyl)-2-pyridyl]-1H-pyrazol-4-yl}propenoate (7.59 g, yield 95%) was obtained as colorless crystals from a fraction eluted with ethyl acetate-hexane (1:4, volume ratio). The crystals were recrystallized from ethyl acetate-hexane. melting point: 75-76° C.

### Reference Example 178

A mixture of ethyl (E)-3-{3-(1-methylpropyl)-1-[5-(trifluoromethyl)-2-pyridyl]-1H-pyrazol-4-yl}propenoate (7.30 g), 5% palladium-carbon (1.48 g) and tetrahydrofuran (50 ml) was stirred at room temperature for 1 hour under a hydrogen atmosphere. Palladium-carbon was removed by filtration and the filtrate was concentrated. The residue was subjected to silica gel column chromatography, and ethyl 3-{3-(1-methylpropyl)-1-[5-(trifluoromethyl)-2-pyridyl]-1H-pyrazol-4-yl}propionate (7.21 g, yield 98%) was obtained as a colorless oil from a fraction eluted with ethyl acetate-hexane (1:2, volume ratio).

¹H-NMR (CDCl₃)δ: 0.92 (3H, t, J=7.2 Hz), 1.20-1.34 (6H, m), 1.54-1.90 (2H, m), 2.58-2.68 (2H, m), 2.76-2.87 (3H, m), 4.16 (2H, q, J=7.2 Hz), 7.90-8.05 (2H, m), 8.28 (1H, s), 8.57-8.63 (1H, m).

### Reference Example 179

To a solution of ethyl 3-{3-(1-methylpropyl)-1-[5-(trifluoromethyl)-2-pyridyl]-1H-pyrazol-4-yl}propionate (7.20 g) in tetrahydrofuran (50 ml) was dropwise added a 1.0 M solution (50 ml) of diisobutylaluminum hydride in hexane at 0° C., and the mixture was stirred at room temperature for 1 hour. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and 3-{3-(1-methylpropyl)-1-[5-(trifluoromethyl)-2-pyridyl]-1H-pyrazol-4-yl}-1-propanol (6.09 g, yield 95%) was obtained as colorless crystals from a fraction eluted with ethyl acetate-hexane (1:1, volume ratio). The crystals were recrystallized from ethyl acetate-hexane. melting point: 72-73° C.

### Reference Example 180

A mixture of 2-methylpentanoic acid (11.65 g), 1,1′-carbonyldiimidazole (17.89 g) and tetrahydrofuran (200 ml) was refluxed for 1.5 hours. After cooling to room temperature, magnesium chloride (10.48 g) and potassium ethoxycarbonylacetate (18.75 g) were added and the mixture was refluxed for 1.5 hours. The reaction solution was acidified with dilute hydrochloric acid, and extracted with ethyl acetate. The ethyl acetate layer was washed with saturated aqueous sodium chloride solution, dried (MgSO₄) and concentrated. A mixture of the residue and N,N-dimethylformamide dimethylacetal (17.90 g) was refluxed for 1 hour, and concentrated under reduced pressure. The residue was dissolved in ethanol (200 ml), and a solution of hydrazine monohydrate (5.10 g) in ethanol (50 ml) was slowly added at room temperature. The mixture was stirred overnight. The reaction mixture was concentrated under reduced pressure and the residue was dissolved in ethyl acetate. The obtained ethyl acetate solution was washed with saturated aqueous sodium chloride solution, dried (MgSO₄) and concentrated. The residue was subjected to silica gel column chromatography, and ethyl 3-(1-methylbutyl)-1H-pyrazole-4-carboxylate (16.85 g, yield 80%) was obtained as a colorless oil from a fraction eluted with ethyl acetate-hexane (1:2, volume ratio).

¹H-NMR (CDCl₃)δ: 0.90 (3H, t, J=7.0 Hz), 1.18-1.44 (6H, m), 1.48-1.80 (4H, m), 3.52-3.70 (1H, m), 4.30 (2H, q, J=7.0 Hz), 7.94 (1H, s).

### Reference Example 181

A mixture of ethyl 3-(1-methylbutyl)-1H-pyrazole-4-carboxylate (6.50 g), 2-chloro-5-(trifluoromethyl)pyridine (5.85 g), potassium carbonate (5.09 g) and N,N-dimethylformamide (100 ml) was stirred overnight at 100° C. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and ethyl 3-(1-methylbutyl)-1-[5-(trifluoromethyl)-2-pyridyl]-1H-pyrazole-4-carboxylate (9.71 g, yield 88%) was obtained as a colorless oil from a fraction eluted with ethyl acetate-hexane (1:4, volume ratio).

¹H-NMR (CDCl₃)δ: 0.91 (3H, t, J=7.2 Hz), 1.23-1.92 (10H, m), 3.44-3.59 (1H, m), 4.32 (2H, q, J=7.2 Hz), 8.00-8.15 (2H, m), 8.65-8.69 (1H, m), 8.97 (1H, s).

### Reference Example 182

To a solution of ethyl 3-(1-methylbutyl)-1-[5-(trifluoromethyl)-2-pyridyl]-1H-pyrazole-4-carboxylate (9.71 g) in tetrahydrofuran (100 ml) was dropwise added a 1.0 M solution (60 ml) of diisobutyl aluminum hydride in hexane at 0° C., and the mixture was stirred at room temperature for 1 hour. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and {3-(1-methylbutyl)-1-[5-(trifluoromethyl)-2-pyridyl]-1H-pyrazol-4-yl}methanol (8.21 g, yield 96%) was obtained as a colorless oil from a fraction eluted with ethyl acetate-hexane (1:1, volume ratio).

¹H-NMR (CDCl₃)δ: 0.91 (3H, t, J=6.8 Hz), 1.27-1.90 (8H, m), 2.88-3.10 (1H, m), 4.65 (2H, d, J=6.2 Hz), 7.93-8.10 (2H, m), 8.48 (1H, s), 8.60-8.66 (1H, m).

### Reference Example 183

A mixture of {3-(1-methylbutyl)-1-[5-(trifluoromethyl)-2-pyridyl]-1H-pyrazol-4-yl}methanol (8.21 g), activated manganese dioxide (26.48 g) and tetrahydrofuran (100 ml) was stirred overnight at room temperature. The insoluble material was removed by filtration and the filtrate was concentrated. The residue was subjected to silica gel column chromatography, and 3-(1-methylbutyl)-1-[5-(trifluoromethyl)-2-pyridyl]-1H-pyrazole-4-carbaldehyde (7.56 g, yield 93%) was obtained as colorless crystals from a fraction eluted with ethyl acetate-hexane (1:4, volume ratio). The crystals were recrystallized from ethyl acetate-hexane. melting point: 63-64° C.

### Reference Example 184

To a mixture of 3-(1-methylbutyl)-1-[5-(trifluoromethyl)-2-pyridyl]-1H-pyrazole-4-carbaldehyde (7.40 g), ethyl diethylphosphonoacetate (5.50 g) and N,N-dimethylformamide (70 ml) was added sodium hydride (60%, in oil, 0.96 g) at 0° C., and the mixture was stirred overnight at room temperature. The reaction mixture was poured into water, and extracted with ethyl acetate. The ethyl acetate layer was washed with dilute hydrochloric acid and then with saturated aqueous sodium chloride solution, dried (MgSO₄) and concentrated. The residue was subjected to silica gel column chromatography, and ethyl (E)-3-{3-(1-methylbutyl)-1-[5-(trifluoromethyl)-2-pyridyl]-1H-pyrazol-4-yl}propenoate (8.15 g, yield 90%) was obtained as a colorless oil from a fraction eluted with ethyl acetate-hexane (1:4, volume ratio).

¹H-NMR (CDCl₃)δ: 0.92 (3H, t, J=7.2 Hz), 1.24-1.45 (8H, m), 1.56-1.88 (2H, m), 2.98-3.14 (1H, m), 4.27 (2H, q, J=7.2 Hz), 6.29 (1H, d, J=16.2 Hz), 7.62 (1H, d, J=16.2 Hz), 7.98-8.13 (2H, m), 8.64-8.70 (1H, m), 8.76 (1H, s).

### Reference Example 185

A mixture of ethyl (E)-3-{3-(1-methylbutyl)-1-[5-(trifluoromethyl)-2-pyridyl]-1H-pyrazol-4-yl}propenoate (8.15 g), 5% palladium-carbon (1.33 g) and tetrahydrofuran (75 ml) was stirred at room temperature for 1 hour under a hydrogen atmosphere. Palladium-carbon was removed by filtration and the filtrate was concentrated. The residue was subjected to silica gel column chromatography, and ethyl 3-{3-(1-methylbutyl)-1-[5-(trifluoromethyl)-2-pyridyl]-1H-pyrazol-4-yl}propionate (8.10 g, yield 99%) was obtained as a colorless oil from a fraction eluted with ethyl acetate-hexane (1:2, volume ratio).

¹H-NMR (CDCl₃)δ: 0.91 (3H, t, J=7.4 Hz), 1.22-1.90 (10H, m), 2.58-2.68 (2H, m), 2.76-2.98 (3H, m), 4.16 (2H, q, J=7.0 Hz), 7.90-8.06 (2H, m), 8.28 (1H, s), 8.58-8.63 (1H, m).

### Reference Example 186

To a solution of ethyl 3-{3-(1-methylbutyl)-1-[5-(trifluoromethyl)-2-pyridyl]-1H-pyrazol-4-yl}propionate (8.10 g) in tetrahydrofuran (50 ml) was dropwise added a 1.0 M solution (50 ml) of diisobutylaluminum hydride in hexane at 0° C., and the mixture was stirred at room temperature for 1 hour. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and 3-{3-(1-methylbutyl)-1-[5-(trifluoromethyl)-2-pyridyl]-1H-pyrazol-4-yl}-1-propanol (6.63 g, yield 92%) was obtained as colorless crystals from a fraction eluted with ethyl acetate-hexane (1:1, volume ratio). The crystals were recrystallized from ethyl acetate-hexane. melting point: 72-73° C.

### Reference Example 187

To a solution of 3-isopropyl-4-[3-(methoxymethoxy)propyl]-1H-pyrazole (0.90 g) in N,N-dimethylformamide (30 ml) was added sodium hydride (60%, in oil, 0.17 g) at 0° C. and the mixture was stirred at room temperature for 15 minutes. 2,3-Dichloro-5-(trifluoromethyl)pyridine (0.93 g) was added at room temperature and the mixture was stirred overnight at 50° C. The reaction mixture was poured into water, and extracted with ethyl acetate. The ethyl acetate layer was washed with saturated aqueous sodium chloride solution, dried (MgSO₄) and concentrated. The residue was subjected to silica gel column chromatography, and 1-[3-chloro-5-(trifluoromethyl)-2-pyridyl]-3-isopropyl-4-[3-(methoxymethoxy)propyl]-1H-pyrazole (1.59 g, yield 96%) was obtained as a colorless oil from a fraction eluted with ethyl acetate-hexane (1:4, volume ratio).

¹H-NMR (CDCl₃)δ: 1.35 (6H, d, J=7.0 Hz), 1.88-2.00 (2H, m), 2.55-2.66 (2H, m), 2.97-3.15 (1H, m), 3.38 (3H, s), 3.58-3.67 (2H, m), 4.65 (2H, s), 8.01 (1H, s), 8.02-8.09 (1H, m), 8.57-8.61 (1H, m).

### Reference Example 188

A mixture of 1-[3-chloro-5-(trifluoromethyl)-2-pyridyl]-3-isopropyl-4-[3-(methoxymethoxy)propyl]-1H-pyrazole (1.59 g), conc. hydrochloric acid (0.05 ml) and methanol (50 ml) was refluxed for 2 hours. The mixture was concentrated under reduced pressure and the residue was dissolved in ethyl acetate. An ethyl acetate solution was washed with saturated aqueous sodium chloride solution, dried (MgSO₄) and concentrated. The residue was subjected to silica gel column chromatography, and 3-{1-[3-chloro-5-(trifluoromethyl)-2-pyridyl]-3-isopropyl-1H-pyrazol-4-yl}-1-propanol (1.33 g, yield 94%) was obtained as colorless crystals from a fraction eluted with ethyl acetate-hexane (1:4, volume ratio). The crystals were recrystallized from ethyl acetate-hexane. melting point: 66-67° C.

### Reference Example 189

To a solution of 3-isopropyl-4-[3-(methoxymethoxy)propyl]-1H-pyrazole (0.98 g) in N,N-dimethylformamide (30 ml) was added sodium hydride (60%, in oil, 0.19 g) at 0° C. and the mixture was stirred at room temperature for 15 minutes. 2,5-Dibromopyridine (1.15 g) was added at room temperature, and the mixture was stirred overnight at 100° C. The reaction mixture was poured into water, and extracted with ethyl acetate. The ethyl acetate layer was washed with saturated aqueous sodium chloride solution, dried (MgSO₄) and concentrated. The residue was subjected to silica gel column chromatography, and 1-(5-bromo-2-pyridyl)-3-isopropyl-4-[3-(methoxymethoxy)propyl]-1H-pyrazole (1.63 g, yield 96%) was obtained as a colorless oil from a fraction eluted with ethyl acetate-hexane (1:4, volume ratio).

¹H-NMR (CDCl₃)δ: 1.32 (6H, d, J=7.0 Hz), 1.84-2.02 (2H, m), 2.52-2.64 (2H, m), 2.94-3.10 (1H, m), 3.38 (3H, s), 3.55-3.66 (2H, m), 4.65 (2H, s), 7.81-7.85 (2H, m), 8.19 (1H, s), 8.36-8.39 (1H, m).

### Reference Example 190

A mixture of 1-(5-bromo-2-pyridyl)-3-isopropyl-4-[3-(methoxymethoxy)propyl]-1H-pyrazole (1.63 g), conc. hydrochloric acid (0.05 ml) and methanol (50 ml) was refluxed for 2 hours. The mixture was concentrated under reduced pressure and the residue was dissolved in ethyl acetate. An ethyl acetate solution was washed with saturated aqueous sodium chloride solution, dried (MgSO₄) and concentrated. The residue was subjected to silica gel column chromatography, and 3-[1-(5-bromo-2-pyridyl)-3-isopropyl-1H-pyrazol-4-yl]-1-propanol (1.32 g, yield 92%) was obtained as colorless crystals from a fraction eluted with ethyl acetate-hexane (1:4, volume ratio). The crystals were recrystallized from ethyl acetate-hexane. melting point: 96-97° C.

### Reference Example 191

A mixture of ethyl 3-(1-ethylpropyl)-1H-pyrazole-4-carboxylate (41.42 g), benzyl bromide (25 ml), potassium carbonate (30.00 g) and N,N-dimethylformamide (200 ml) was stirred overnight at room temperature. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and ethyl 1-benzyl-3-(1-ethylpropyl)-1H-pyrazole-4-carboxylate (55.62 g, yield 94%) was obtained as a colorless oil from a fraction eluted with ethyl acetate-hexane (1:4, volume ratio).

¹H-NMR (CDCl₃)δ: 0.84 (6H, t, J=7.2 Hz), 1.31 (3H, t, J=7.2 Hz), 1.60-1.88 (4H, m), 3.14-3.32 (1H, m), 4.23 (2H, q, J=7.2 Hz), 5.27 (2H, s), 7.10-7.40 (5H, m), 7.86 (1H, s).

### Reference Example 192

To a solution of ethyl 1-benzyl-3-(1-ethylpropyl)-1H-pyrazole-4-carboxylate (55.62 g) in tetrahydrofuran (200 ml) was added lithium aluminum hydride (5.38 g) at 0° C., and the mixture was stirred at room temperature for 1 hour. Sodium sulfate 10 hydrate (53.88 g) was added to the reaction mixture, and the mixture was stirred at room temperature for 1 hour. The precipitate was removed by filtration and the filtrate was concentrated. The residue was subjected to silica gel column chromatography, and [1-benzyl-3-(1-ethylpropyl)-1H-pyrazol-4-yl]methanol (47.18 g, yield 99%) was obtained as a colorless oil from a fraction eluted with ethyl acetate.

¹H-NMR (CDCl₃)δ: 0.84 (6H, t, J=7.4 Hz), 1.22 (1H, br t), 1.60-1.82 (4H, m), 2.48-2.70 (1H, m), 4.52 (2H, d, J=5.0 Hz), 5.26 (2H, s), 7.08-7.42 (6H, m).

### Reference Example 193

A mixture of [1-benzyl-3-(1-ethylpropyl)-1H-pyrazol-4-yl]methanol (47.18 g), activated manganese dioxide (152.00 g) and tetrahydrofuran (300 ml) was stirred overnight at room temperature. The insoluble material was removed by filtration and the filtrate was concentrated. The residue was subjected to silica gel column chromatography, and 1-benzyl-3-(1-ethylpropyl)-1H-pyrazole-4-carbaldehyde (42.25 g, yield 90%) was obtained as a colorless oil from a fraction eluted with ethyl acetate-hexane (1:4, volume ratio).

¹H-NMR (CDCl₃)δ: 0.85 (6H, t, J=7.4 Hz), 1.67-1.90 (4H, m), 2.88-3.10 (1H, m), 5.29 (2H, s), 7.18-7.41 (5H, m), 7.76 (1H, s), 9.87 (1H, s).

### Reference Example 194

To a mixture of 1-benzyl-3-(1-ethylpropyl)-1H-pyrazole-4-carbaldehyde (42.25 g), ethyl diethylphosphonoacetate (40.70 g) and N,N-dimethylformamide (200 ml) was added sodium hydride (60%, in oil, 6.95 g) at 0° C. and the mixture was stirred overnight at room temperature. The reaction mixture was poured into water, and extracted with ethyl acetate. The ethyl acetate layer was washed with dilute hydrochloric acid and then with saturated aqueous sodium chloride solution, dried (MgSO₄) and concentrated. The residue was subjected to silica gel column chromatography, and ethyl (E)-3-[1-benzyl-3-(1-ethylpropyl)-1H-pyrazol-4-yl]propenoate (52.30 g, yield 97%) was obtained as a colorless oil from a fraction eluted with ethyl acetate-hexane (1:4, volume ratio).

¹H-NMR (CDCl₃)δ: 0.83 (6H, t, J=7.2 Hz), 1.30 (3H, t, J=7.2 Hz), 1.60-1.84 (4H, m), 2.64-2.78 (1H, m), 4.21 (2H, q, J=7.2 Hz), 5.27 (2H, s), 6.02 (1H, d, J=15.6 Hz), 7.08-7.42 (5H, m), 7.51 (1H, s), 7.57 (1H, d, J=15.6 Hz).

### Reference Example 195

A mixture of ethyl (E)-3-[1-benzyl-3-(1-ethylpropyl)-1H-pyrazol-4-yl]propenoate (10.00 g), 5% palladium-carbon (10.26 g), formic acid (50 ml) and ethanol (50 ml) was refluxed for 5 hours. Palladium-carbon was removed by filtration and the filtrate was concentrated. The residue was dissolved in ethyl acetate, washed with saturated aqueous sodium chloride solution, dried (MgSO₄) and concentrated. The residue was subjected to silica gel column chromatography, and ethyl 3-[3-(1-ethylpropyl)-1H-pyrazol-4-yl]propionate (6.60 g, yield 91%) was obtained as a colorless oil from a fraction eluted with ethyl acetate-hexane (1:1, volume ratio).

¹H-NMR (CDCl₃)δ: 0.82 (6H, t, J=7.4 Hz), 1.25 (3H, t, J=7.4 Hz), 1.50-1.82 (4H, m), 2.48-2.81 (5H, m), 4.14 (2H, q, J=7.4 Hz), 7.36 (1H, s).

### Reference Example 196

To a solution of 3-isopropyl-4-[3-(methoxymethoxy)propyl]-1H-pyrazole (0.90 g) in N,N-dimethylformamide (30 ml) was added sodium hydride (60%, in oil, 0.19 g) at 0° C., and the mixture was stirred at room temperature for 30 minutes. 2,3,5-Trichloropyridine (0.89 g) was added at room temperature, and the mixture was stirred at room temperature for 3 hours. The reaction mixture was poured into water, and extracted with ethyl acetate. The ethyl acetate layer was washed with saturated aqueous sodium chloride solution, dried (MgSO₄) and concentrated. The residue was subjected to silica gel column chromatography, and 1-(3,5-dichloro-2-pyridyl)-3-isopropyl-4-[3-(methoxymethoxy)propyl]-1H-pyrazole (1.19 g, yield 78%) was obtained as a colorless oil from a fraction eluted with ethyl acetate-hexane (1:5, volume ratio).

¹H-NMR (CDCl₃)δ: 1.34 (6H, d, J=7 Hz), 1.85-2.00 (2H, m), 2.55-2.65 (2H, m), 2.95-3.15 (1H, m), 3.38 (3H, s), 3.62 (2H, t, J=6 Hz), 4.65 (2H, s), 7.84 (1H, s), 7.86 (1H, d, J=2 Hz), 8.35 (1H, d, J=2 Hz).

### Reference Example 197

A mixture of 1-(3,5-dichloro-2-pyridyl)-3-isopropyl-4-[3-(methoxymethoxy)propyl]-1H-pyrazole (1.18 g), conc. hydrochloric acid (0.1 ml) and methanol (20 ml) was refluxed for 2 hours. The mixture was concentrated under reduced pressure and the residue was dissolved in ethyl acetate. An ethyl acetate solution was washed with saturated aqueous sodium chloride solution, dried (MgSO₄) and concentrated to give 3-[1-(3,5-dichloro-2-pyridyl)-3-isopropyl-1H-pyrazol-4-yl]-1-propanol (1.02 g, yield 99%) as a colorless oil.

¹H-NMR (CDCl₃)δ: 1.34 (6H, d, J=7 Hz), 1.80-2.00 (2H, m), 2.55-2.65 (2H, m), 2.95-3.15 (1H, m), 3.70-3.80 (2H, m), 7.84 (1H, s), 7.86 (1H, d, J=2 Hz), 8.35 (1H, d, J=2 Hz).

### Reference Example 198

To a mixture of sodium ethoxide (39.58 g) and diisopropyl ether (800 ml) was added a mixture of ethyl valerate (74.21 g) and ethyl formate (50.67 g) at 0° C. over 1 hour. The mixture was stirred at room temperature overnight. Acetic acid (66 ml) was added to the reaction mixture over 20 minutes and then hydrazine monohydrate (32.0 g) was added over 10 minutes. The mixture was refluxed for 2 hours. Water (150 ml) was added to the reaction mixture and the mixture was stirred at 0° C. for 1 hour. The precipitated crystals were collected by filtration, washed with cold water and isopropyl ether, and dried to give gray-white crystals. To a mixture of the obtained crystals, triethylamine (10.1 ml) and tetrahydrofuran (70 ml) was added di-tert-butyl dicarbonate (16.7 ml) and the mixture was stirred overnight at room temperature. The reaction solution was concentrated and water was added to the residue. The resulting crystals were collected by filtration, washed with water and hexane, and dried to give tert-butyl 3-hydroxy-4-propyl-1H-pyrazole-1-carboxylate (10.30 g, yield 66%) as white crystals. melting point: 70-71° C. (decomposition).

¹H-NMR (CDCl₃)δ: 0.95 (3H, t, J=7.3 Hz), 1.55-1.65 (11H, m), 2.35 (2H, t, J=7.4 Hz), 7.62 (1H, br s).

### Reference Example 199

To a mixture of 3-{3-propyl-1-[5-(trifluoromethyl)-2-pyridyl]-1H-pyrazol-4-yl}-1-propanol (660 mg), tert-butyl 3-hydroxy-4-propyl-1H-pyrazole-1-carboxylate (530 mg), tributylphosphine (860 mg) and tetrahydrofuran (30 ml) was added 1,1′-azodicarbonyldipiperidine (1.06 g) at room temperature and the mixture was stirred overnight. The reaction solution was concentrated. The residue was subjected to silica gel column chromatography, and a colorless oil was obtained from a fraction eluted with diethyl ether-hexane (1:4, volume ratio). A mixture of the obtained oily substance and 4N hydrogen chloride ethyl acetate solution (10 ml) was stirred overnight at room temperature. The reaction mixture was poured into saturated aqueous sodium hydrogen carbonate and extracted with ethyl acetate. The ethyl acetate layer was washed with saturated aqueous sodium chloride solution, dried (MgSO₄) and concentrated. The obtained colorless crystals were collected by filtration to give 4-propyl-3-(3-{3-propyl-1-[5-(trifluoromethyl)-2-pyridyl]-1H-pyrazol-4-yl}propoxy)-1H-pyrazole (700 mg, yield 79%). melting point: 127-128° C.

### Reference Example 200

To a mixture of 3-{3-ethoxy-1-[5-(trifluoromethyl)-2-pyridyl]-1H-pyrazol-4-yl}-1-propanol (1.00 g), tert-butyl 3-hydroxy-4-propyl-1H-pyrazole-1-carboxylate (790 mg), tributylphosphine (1.31 g) and tetrahydrofuran (50 ml) was added 1,1′-azodicarbonyldipiperidine (1.64 g) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance and 4N hydrogen chloride ethyl acetate solution (20 ml) was stirred overnight at room temperature. The reaction mixture was poured into saturated aqueous sodium hydrogen carbonate and extracted with ethyl acetate. The ethyl acetate layer was washed with saturated aqueous sodium chloride solution, dried (MgSO₄) and concentrated. The obtained colorless crystals were collected by filtration to give 3-(3-{3-ethoxy-1-[5-(trifluoromethyl)-2-pyridyl]-1H-pyrazol-4-yl}propoxy)-4-propyl-1H-pyrazole (1.19 g, yield 89%). melting point: 121-122° C.

¹H-NMR (CDCl₃)δ: 0.94 (3H, t, J=7.3 Hz), 1.42 (3H, t, J=7.1 Hz), 1.57 (2H, sextet, J=7.4 Hz), 2.09 (2H, quintet, J=7.0 Hz), 2.34 (2H, t, J=7.4 Hz), 2.59 (2H, t, J=7.4 Hz), 4.24 (2H, t, J=6.3 Hz), 4.35 (2H, q, J=7.0 Hz), 7.14 (1H, s), 7.80 (1H, d, J=8.5 Hz), 7.90 (1H, dd, J=8.8, 2.2 Hz), 8.20 (1H, s), 8.53-8.55 (1H, m), 8.82 (1H, br s).

### Reference Example 201

A mixture of cyclohexylhydrazine hydrochloride (20.12 g), dimethyl acetylenedicarboxylate (19.00 g), potassium acetate (13.11 g), acetic acid (70 ml) and toluene (70 ml) was stirred at 80° C. for 3 hours. The reaction mixture was poured into water, and extracted with ethyl acetate. The ethyl acetate layer was washed with saturated aqueous sodium chloride solution, dried (MgSO₄) and concentrated. Toluene was added to the residue, and the resulting solid was removed by filtration. The filtrate was concentrated. The residue was subjected to silica gel column chromatography, and methyl 1-cyclohexyl-3-hydroxy-1H-pyrazole-5-carboxylate (11.86 g, yield 40%) was obtained as colorless crystals from a fraction eluted with ethyl acetate-chloroform (1:6, volume ratio). melting point: 195-196° C.

¹H-NMR (CDCl₃)δ: 1.23-1.97 (10H, m), 3.87 (3H, s), 5.00-5.10 (1H, m), 6.14 (1H, s), 10.99 (1H, br s).

### Reference Example 202

A mixture of methyl 1-cyclohexyl-3-hydroxy-1H-pyrazole-5-carboxylate (11.00 g), benzyl bromide (6.10 ml), potassium carbonate (6.80 g) and N,N-dimethylformamide (80 ml) was stirred overnight at room temperature. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and methyl 3-benzyloxy-1-cyclohexyl-1H-pyrazole-5-carboxylate (15.40 g, quantitative) was obtained as a colorless oil from a fraction eluted with ethyl acetate-hexane (1:4, volume ratio).

¹H-NMR (CDCl₃)δ: 1.18-1.48 (3H, m), 1.65-1.74 (1H, m), 1.82-1.97 (6H, m), 3.84 (3H, s), 4.94-5.03 (1H, m), 5.17 (2H, s), 6.18 (1H, s), 7.28-7.47 (5H, m).

### Reference Example 203

To a mixture of lithium aluminum hydride (4.65 g) and tetrahydrofuran (100 ml) was slowly added a solution of methyl 3-benzyloxy-1-cyclohexyl-1H-pyrazole-5-carboxylate (15.40 g) in tetrahydrofuran (10 ml) at 0° C., and the mixture was stirred at room temperature for 30 minutes. Acetone (20 ml) was slowly added to decompose excess lithium aluminum hydride, and brine (13 ml) was added. The precipitate was removed by filtration and the filtrate was concentrated. The residue was subjected to silica gel column chromatography, and (3-benzyloxy-1-cyclohexyl-1H-pyrazol-5-yl)methanol (13.61 g, yield 97%) was obtained as colorless crystals from a fraction eluted with ethyl acetate-hexane (2:3, volume ratio). melting point: 195-196° C.

¹H-NMR (CDCl₃)δ: 1.20-1.45 (3H, m), 1.55-1.73 (2H, m), 1.84-2.01 (6H, m), 3.97-4.07 (1H, m), 4.57 (2H, d, J=6.1 Hz), 5.15 (2H, s), 5.59 (1H, s), 7.27-7.47 (5H, m).

### Reference Example 204

A mixture of (3-benzyloxy-1-cyclohexyl-1H-pyrazol-5-yl)methanol (12.50 g), activated manganese dioxide (50.0 g) and tetrahydrofuran (250 ml) was stirred overnight at room temperature. The insoluble material was removed by filtration and the filtrate was concentrated. The residue was subjected to silica gel column chromatography, and a colorless oil was obtained from a fraction eluted with ethyl acetate-hexane (1:3, volume ratio). To a mixture of the obtained oily substance, ethyl diethylphosphonoacetate (6.75 g) and N,N-dimethylformamide (50 ml) was added sodium hydride (60%, in oil, 1.20 g) at 0° C. and the mixture was stirred overnight at room temperature. The reaction mixture was poured into water, and extracted with ethyl acetate. The ethyl acetate layer was washed with saturated aqueous sodium chloride solution, dried (MgSO₄) and concentrated. The residue was subjected to silica gel column chromatography, and ethyl (E)-3-(3-benzyloxy-1-cyclohexyl-1H-pyrazol-5-yl)propenoate (7.72 g, yield 50%) was obtained as a colorless oil from a fraction eluted with ethyl acetate-hexane (1:4, volume ratio).

¹H-NMR (CDCl₃)δ: 1.17-1.49 (6H, m), 1.67-1.76 (1H, m), 1.83-2.02 (6H, m), 4.06-4.15 (1H, m), 4.26 (2H, q, J=7.1 Hz), 5.17 (2H, s), 5.92 (1H, s), 6.27 (1H, d, J=15.9 Hz), 7.28-7.47 (5H, m), 7.55 (1H, d, J=15.9 Hz).

### Reference Example 205

A mixture of ethyl (E)-3-(3-benzyloxy-1-cyclohexyl-1H-pyrazol-5-yl)propenoate (7.70 g), 5% palladium-carbon (1.0 g), tetrahydrofuran (50 ml) and ethanol (50 ml) was stirred overnight at room temperature under a hydrogen atmosphere. Palladium-carbon was removed by filtration and the filtrate was concentrated to give ethyl 3-(1-cyclohexyl-3-hydroxy-1H-pyrazol-5-yl)propanoate (5.54 g, yield 96%) as colorless crystals. melting point: 173-174° C.

### Reference Example 206

To a mixture of methyl acetylenedicarboxylate (29.20 g) and methanol (200 ml) was added hydrazine monohydrate (10.30 g) at 0° C., and the mixture was stirred overnight at room temperature. The reaction mixture was concentrated to give yellow crystals (28.61 g). To a mixture of the obtained crystals, triethylamine (29.5 ml) and tetrahydrofuran (200 ml) was added di-tert-butyl dicarbonate (48.6 ml), and the mixture was stirred overnight. The reaction mixture was concentrated. A mixture of the obtained residue, benzyl bromide, potassium carbonate (29.20 g) and N,N-dimethylformamide (200 ml) was stirred overnight at room temperature. The reaction mixture was poured into dilute hydrochloric acid, and extracted with ethyl acetate. The ethyl acetate layer was washed with saturated aqueous sodium chloride solution, dried (MgSO₄) and concentrated. A mixture of the residue and 4N hydrogen chloride ethyl acetate solution (100 ml) was stirred overnight at room temperature. The reaction mixture was poured into saturated aqueous sodium hydrogen carbonate and extracted with ethyl acetate. The ethyl acetate layer was washed with saturated aqueous sodium chloride solution, dried (MgSO₄) and concentrated. The residue was subjected to silica gel column chromatography, and methyl 3-benzyloxy-1H-pyrazole-5-carboxylate (12.10 g, yield 26%) was obtained as a yellow oily substance from a fraction eluted with ethyl acetate-hexane (2:3, volume ratio).

¹H-NMR (CDCl₃)δ: 3.89 (3H, s), 5.25 (2H, s), 6.26 (1H, s), 7.22-7.47 (5H, m), 10.60 (1H, br s).

### Reference Example 207

To a mixture of methyl 3-benzyloxy-1H-pyrazole-5-carboxylate (12.10 g) and N,N-dimethylformamide (50 ml) was added sodium hydride (60%, in oil, 1.20 g) at 0° C. and the mixture was stirred for 30 minutes. Isopropyl iodide (5.70 ml) was added and the mixture was stirred overnight at room temperature. The reaction mixture was poured into water, and extracted with ethyl acetate. The ethyl acetate layer was washed with saturated aqueous sodium chloride solution, dried (MgSO₄) and concentrated. The residue was subjected to silica gel column chromatography, and methyl 3-benzyloxy-1-(1-methylethyl)-1H-pyrazole-5-carboxylate (7.34 g, yield 51%) was obtained as a colorless oil from a fraction eluted with ethyl acetate-hexane (1:4, volume ratio).

¹H-NMR (CDCl₃)δ: 1.44 (6H, d, J=6.6 Hz), 3.84 (3H, s), 5.18 (2H, s), 5.41 (1H, septet, J=6.6 Hz), 6.18 (1H, s), 7.27-7.47 (5H, m).

### Reference Example 208

To a mixture of lithium aluminum hydride (1.30 g) and tetrahydrofuran (50 ml) was slowly added a solution of methyl 3-benzyloxy-1-(1-methylethyl)-1H-pyrazole-5-carboxylate (7.34 g) in tetrahydrofuran (5 ml) at 0° C., and the mixture was stirred at room temperature for 30 minutes. Acetone (20 ml) was slowly added to decompose excess lithium aluminum hydride, and brine (4 ml) was further added. The precipitate was removed by filtration and the filtrate was concentrated. The residue was subjected to silica gel column chromatography, and [3-benzyloxy-1-(1-methylethyl)-1H-pyrazol-5-yl]methanol (2.63 g, yield 40%) was obtained as a colorless oil from a fraction eluted with acetone-hexane (2:3, volume ratio).

¹H-NMR (CDCl₃)δ: 1.44 (6H, d, J=6.6 Hz), 1.74 (1H, t, J=6.1 Hz), 4.48 (1H, septet, J=6.6 Hz), 4.57 (2H, d, J=5.8 Hz), 5.15 (2H, s), 5.58 (1H, s), 7.24-7.50 (5H, m).

### Reference Example 209

A mixture of [3-benzyloxy-1-(1-methylethyl)-1H-pyrazol-5-yl]methanol (2.60 g), activated manganese dioxide (8.0 g) and tetrahydrofuran (30 ml) was stirred overnight at room temperature. The insoluble material was removed by filtration and the filtrate was concentrated. The residue was subjected to silica gel column chromatography, and a colorless oil was obtained from a fraction eluted with ethyl acetate-hexane (1:3, volume ratio). To a mixture of the obtained oily substance, ethyl diethylphosphonoacetate (1.67 g) and N,N-dimethylformamide (20 ml) was added sodium hydride (60%, in oil, 0.30 g) at 0° C., and the mixture was stirred overnight at room temperature. The reaction mixture was poured into water, and extracted with ethyl acetate. The ethyl acetate layer was washed with saturated aqueous sodium chloride solution, dried (MgSO₄) and concentrated. The residue was subjected to silica gel column chromatography, and ethyl (E)-3-(3-benzyloxy-1-(1-methylethyl)-1H-pyrazol-5-yl)propenoate (1.23 g, yield 37%) was obtained as a colorless oil from a fraction eluted with diethyl ether-hexane (1:4, volume ratio).

¹H-NMR (CDCl₃)δ: 1.33 (3H, t, J=7.1 Hz), 1.46 (6H, d, J=6.6 Hz), 4.26 (2H, q, J=7.2 Hz), 4.57 (1H, septet, J=6.6 Hz), 5.17 (2H, s), 5.92 (1H, s), 6.27 (1H, d, J=15.8 Hz), 7.27-7.50 (5H, m), 7.54 (1H, d, J=15.8 Hz).

### Reference Example 210

A mixture of ethyl (E)-3-(3-benzyloxy-1-(1-methylethyl)-1H-pyrazol-5-yl)propenoate (1.23 g), 5% palladium-carbon (0.2 g) and tetrahydrofuran (10 ml) was stirred overnight at room temperature under a hydrogen atmosphere. Palladium-carbon was removed by filtration and the filtrate was concentrated to give ethyl 3-(3-hydroxy-1-(1-methylethyl)-1H-pyrazol-5-yl)propanoate (0.88 g, quantitative) as colorless crystals. melting point: 123-124° C.

¹H-NMR (CDCl₃)δ: 1.27 (3H, t, J=7.1 Hz), 1.42 (6H, d, J=6.6 Hz), 2.57-2.68 (2H, m), 2.80-2.92 (2H, m), 4.16 (2H, q, J=7.1 Hz), 4.32 (1H, septet, J=6.6 Hz), 5.37 (1H, s).

### Reference Example 211

A mixture of methyl 4-methyl-3-oxopentanoate (20.00 g) nd 1,1-dimethoxytrimethylamine (24.8 g) was refluxed for 2 ours. The reaction mixture was concentrated to give a yellow ily substance. To a mixture of the obtained oily substance and ethanol (200 ml) was added hydrazine monohydrate (7.30 g) at 0° C., and the mixture was stirred at room temperature overnight. The reaction mixture was concentrated, and the residue was dissolved in ethyl acetate, washed with saturated aqueous sodium hydrogen carbonate and brine in this order, dried (MgSO₄) and concentrated to give a brown oily substance. A mixture of the obtained oily substance, benzyl bromide (17.0 ml), potassium carbonate (20.0 g) and N,N-dimethylformamide (200 ml) was stirred at room temperature for 4 hours. The reaction mixture was poured into water, and extracted with ethyl acetate. The ethyl acetate layer was washed with saturated aqueous sodium chloride solution, dried (MgSO₄) and concentrated. The residue was subjected to silica gel column chromatography, and methyl 1-benzyl-3-(1-methylethyl)-1H-pyrazole-4-carboxylate (29.93 g, yield 84%) was obtained as a yellow oily substance from a fraction eluted with diethyl ether-hexane (1:4, volume ratio).

¹H-NMR (CDCl₃)δ: 1.31 (6H, d, J=7.0 Hz), 3.30-3.60 (1H, m), 3.76 (3H, s), 5.24 (2H, s), 7.18-7.40 (5H, m), 7.69 (1H, s).

### Reference Example 212

To a mixture of lithium aluminum hydride (5.50 g) and tetrahydrofuran (260 ml) was slowly added a solution of methyl 1-benzyl-3-(1-methylethyl)-1H-pyrazole-4-carboxylate (29.93 g) in tetrahydrofuran (40 ml) at 0° C., and the mixture was stirred at room temperature for 30 minutes. Acetone (20 ml) was slowly added to decompose excess lithium aluminum hydride and brine (15 ml) was added. The precipitate was removed by filtration and the filtrate was concentrated. The residue was subjected to silica gel column chromatography, and [1-benzyl-3-(1-methylethyl)-1H-pyrazol-4-yl]methanol (25.21 g, yield 94%) was obtained as a colorless oil from a fraction eluted with acetone-hexane (2:3, volume ratio).

¹H-NMR (CDCl₃)δ: 1.32 (6H, d, J=7.0 Hz), 1.45 (1H, br s), 3.08 (1H, septet, J=7.0 Hz), 4.54 (2H, br s), 5.22 (2H, s), 7.14-7.40 (6H, m).

### Reference Example 213

A mixture of [1-benzyl-3-(1-methylethyl)-1H-pyrazol-4-yl]methanol (25.00 g), activated manganese dioxide (100.0 g) and tetrahydrofuran (350 ml) was stirred overnight at room temperature. The insoluble material was removed by filtration and the filtrate was concentrated. The residue was subjected to silica gel column chromatography, and a colorless oil was obtained from a fraction eluted with ethyl acetate-hexane (1:2, volume ratio). To a mixture of the obtained oily substance, ethyl diethylphosphonoacetate (25.80 g) and N,N-dimethylformamide (180 ml) was added sodium hydride (60%, in oil, 4.60 g) at 0° C., and the mixture was stirred overnight at room temperature. The reaction mixture was poured into water, and extracted with ethyl acetate. The ethyl acetate layer was washed with saturated aqueous sodium chloride solution, dried (MgSO₄) and concentrated. The residue was subjected to silica gel column chromatography, and ethyl (E)-3-(1-benzyl-3-(1-methylethyl)-1H-pyrazol-4-yl)propenoate (30.25 g, yield 94%) was obtained as a colorless oil from a fraction eluted with ethyl acetate-hexane (1:4, volume ratio).

¹H-NMR (CDCl₃)δ: 1.30 (3H, t, J=7.3 Hz), 1.33 (6H, d, J=6.8 Hz), 3.16 (1H, septet, J=6.8 Hz), 4.21 (2H, q, J=7.2 Hz), 5.25 (2H, s), 6.51 (1H, d, J=16.0 Hz), 7.18-7.40 (5H, m), 7.45 (1H, s), 7.58 (1H, d, J=16.0 Hz).

### Reference Example 214

To a mixture of 2-ethylphenol (12.22 g), tributylamine (7.41 g) and toluene (50 ml) was added tin tetrachloride (2.61 g) and the mixture was stirred at room temperature for 30 minutes. Paraformaldehyde (6.60 g) was added and the mixture was stirred overnight at 100° C. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and 3-ethylsalicylaldehyde (8.20 g, yield 55%) was obtained as a colorless oil from a fraction eluted with hexane.

¹H-NMR (CDCl₃)δ: 1.23 (3H, t, J=7.6 Hz), 2.70 (2H, q, J=7.6 Hz), 6.96 (1H, t, J=7.6 Hz), 7.37-7.42 (2H, m), 9.89 (1H, s), 11.28 (1H, s).

### Reference Example 215

To a mixture of lithium aluminum hydride (2.00 g) and tetrahydrofuran (50 ml) was slowly added a solution of ethyl 3-[1-benzyl-3-(1-methylethyl)-1H-pyrazol-4-yl]propanoate (11.73 g) in tetrahydrofuran (10 ml) at 0° C., and the mixture was stirred at room temperature for 30 minutes. Acetone (20 ml) was slowly added to decompose excess lithium aluminum hydride, and brine (5.5 ml) was added. The precipitate was removed by filtration and the filtrate was concentrated. The residue was subjected to silica gel column chromatography, and 3-[1-benzyl-3-(1-methylethyl)-1H-pyrazol-4-yl]-1-propanol (9.95 g, yield 98%) was obtained as a colorless oil from a fraction eluted with acetone-hexane (2:3, volume ratio).

¹H-NMR (CDCl₃)δ: 1.29 (6H, d, J=7.0 Hz), 1.44 (1H, t, J=5.3 Hz), 1.70-1.85 (2H, m), 2.49 (2H, t, J=7.7 Hz), 2.98 (1H, septet, J=7.0 Hz), 3.67 (2H, d, J=5.9 Hz), 5.22 (2H, s), 7.02 (1H, s), 7.13-7.39 (5H, m).

### Reference Example 216

To a mixture of 3-[1-benzyl-3-(1-methylethyl)-1H-pyrazol-4-yl]-1-propanol (9.95 g), N-ethyldiisopropylamine (10.0 ml) and tetrahydrofuran (100 ml) was added chloromethyl methyl ether (5.50 ml) at 0° C. and the mixture was stirred at room temperature overnight. The reaction mixture was poured into water, and extracted with ethyl acetate. The ethyl acetate layer was washed with saturated aqueous sodium chloride solution, dried (MgSO₄) and concentrated. The residue was subjected to silica gel column chromatography, and 1-benzyl-4-[3-(methoxymethoxy)propyl]-3-(1-methylethyl)-1H-pyrazole (10.57 g, yield 91%) was obtained as a colorless oil from a fraction eluted with ethyl acetate-hexane (2:3, volume ratio).

¹H-NMR (CDCl₃)δ: 1.29 (6H, d, J=7.0 Hz), 1.70-1.88 (2H, m), 2.49 (2H, t, J=7.7 Hz), 2.98 (1H, septet, J=7.0 Hz), 3.34 (3H, s), 3.54 (2H, t, J=6.4 Hz), 4.61 (2H, s), 5.22 (2H, s), 7.01 (1H, s), 7.12-7.38 (5H, m).

### Reference Example 217

A mixture of 1-benzyl-4-[3-(methoxymethoxy)propyl]-3-(1-methylethyl)-1H-pyrazole (10.57 g), 5% palladium-carbon (2.0 g) and tetrahydrofuran (100 ml) was stirred overnight at 50° C. under a hydrogen atmosphere. Palladium-carbon was removed by filtration and the filtrate was concentrated to give 4-[3-(methoxymethoxy)propyl]-3-(1-methylethyl)-1H-pyrazole (7.44 g, quantitative) as a yellow oily substance.

¹H-NMR (CDCl₃)δ: 1.29 (6H, d, J=7.0 Hz), 1.77-1.94 (2H, m), 2.53 (2H, t, J=7.7 Hz), 3.05 (1H, septet, J=7.0 Hz), 3.38 (3H, s), 3.57 (2H, t, J=6.4 Hz), 4.64 (2H, s), 7.34 (1H, s).

### Reference Example 218

To a mixture of ethyl 3-[3-(1-methylethyl)-1H-pyrazol-4-yl]propanoate (1.00 g), 2-chloro-5-nitropyridine (0.79 g) and N,N-dimethylformamide (10 ml) was added sodium hydride (60%, in oil, 0.25 g) at 0° C., and the mixture was stirred overnight at room temperature. The reaction mixture was poured into water, and extracted with ethyl acetate. The ethyl acetate layer was washed with saturated aqueous sodium chloride-solution, dried (MgSO₄) and concentrated. The residue was subjected to silica gel column chromatography, and ethyl 3-[3-(1-methylethyl)-1-(5-nitro-2-pyridyl)-1H-pyrazol-4-yl]propanoate (1.26 g, yield 74%) was obtained as yellow crystals from a fraction eluted with ethyl acetate-hexane (1:4, volume ratio). melting point: 90-91° C.

¹H-NMR (CDCl₃)δ: 1.27 (3H, t, J=7.2 Hz), 1.34 (6H, d, J=7.0 Hz), 2.60-2.72 (2H, m), 2.78-2.90 (2H, m), 3.04 (1H, septet, J=6.9 Hz), 4.17 (2H, q, J=7.2 Hz), 8.05 (1H, d, J=9.0 Hz), 8.30 (1H, s), 8.50 (1H, dd, J=9.2, 2.6 Hz), 9.20 (1H, dd, J=2.6, 0.6 Hz).

### Reference Example 219

A mixture of ethyl 3-[3-(1-methylethyl)-1-(5-nitro-2-pyridyl)-1H-pyrazol-4-yl]propanoate (1.18 g), 5% palladium-carbon (0.15 g), methanol (4 ml) and tetrahydrofuran (4 ml) was stirred overnight at room temperature under a hydrogen atmosphere. Palladium-carbon was removed by filtration and the filtrate was concentrated to give ethyl 3-[1-(5-amino-2-pyridyl)-3-(1-methylethyl)-1H-pyrazol-4-yl]propanoate (0.93 g, yield 94%) as yellow crystals. melting point: 75-76° C.

¹H-NMR (CDCl₃)δ: 1.26 (3H, t, J=7.1 Hz), 1.32 (6H, d, J=6.9 Hz), 2.57-2.65 (2H, m), 2.77-2.85 (2H, m), 3.03 (1H, septet, J=6.9 Hz), 3.63 (2H, br s), 4.14 (2H, q, J=7.2 Hz), 7.09 (1H, dd, J=8.9, 2.9 Hz), 7.70 (1H, dd, J=8.6, 0.8 Hz), 7.82 (1H, dd, J=3.0, 0.6 Hz), 8.09 (1H, s).

### Reference Example 220

To a mixture of ethyl 3-[1-(5-amino-2-pyridyl)-3-(1-methylethyl)-1H-pyrazol-4-yl]propanoate (2.00 g), tetrafluoroboric acid (42%, 4 ml) and 1,4-dioxane (3 ml) was slowly added a solution of sodium nitrite (0.50 g) in water (1 ml) at 0° C. and the mixture was stirred for 30 minutes. Cold water (30 ml) was added to the reaction mixture, and the precipitated crystals were collected by filtration, washed with water and air-dried. The obtained crystal was slowly added to toluene (15 ml) heated to 90° C., and the mixture was stirred at 100° C. for 30 minutes. The reaction mixture was poured into water, and extracted with ethyl acetate. The ethyl acetate layer was washed with saturated aqueous sodium chloride solution, dried (MgSO₄) and concentrated. The residue was subjected to silica gel column chromatography, and a yellow oily substance (1.17 g) was obtained from a fraction eluted with ethyl acetate-hexane (1:4, volume ratio). To a mixture of the obtained oily substance and tetrahydrofuran (15 ml) was slowly added a 1.5M solution (6.5 ml) of diisobutylaluminum hydride in toluene at 0° C., and the mixture was stirred at room temperature for 1 hour. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and 3-[1-(5-fluoro-2-pyridyl)-3-(1-methylethyl)-1H-pyrazol-4-yl]-1-propanol (0.74 g, yield 42%) was obtained as colorless crystals from a fraction eluted with ethyl acetate-hexane (1:2, volume ratio). melting point: 78-79° C.

¹H-NMR (CDCl₃)δ: 1.32 (6H, d, J=6.9 Hz), 1.83-1.98 (2H, m), 2.58 (2H, t, J=7.8 Hz), 3.02 (1H, septet, J=6.9 Hz), 3.74 (2H, t, j=5.6 Hz), 7.42-7.52 (1H, m), 7.88-7.95 (1H, m), 8.14-8.20 (2H, m).

### Reference Example 221

To a mixture of 4-[3-(methoxymethoxy)propyl]-3-(1-methylethyl)-1H-pyrazole (0.50 g), 6-chloropyridine-3-carbonitrile (0.36 g) and N,N-dimethylformamide (6 ml) was added sodium hydride (60%, in oil, 0.12 g) at 0° C., and, after termination of hydrogen generation, the mixture was stirred at 80° C. for 3 hours. The reaction mixture was poured into water, and extracted with ethyl acetate. The ethyl acetate layer was washed with saturated aqueous sodium chloride solution, dried (MgSO₄) and concentrated. A mixture of the obtained residue, conc. hydrochloric acid (2 drops) and methanol (6 ml) was stirred overnight at 60° C. The reaction mixture was poured into aqueous sodium hydrogen carbonate, and the precipitated crystals were collected by filtration, washed with water and dried to give 6-[4-(3-hydroxypropyl)-3-(1-methylethyl)-1H-pyrazol-1-yl]pyridine-3-carbonitrile (550 mg, yield 90%) as colorless crystals. melting point: 105-106° C.

¹H-NMR (CDCl₃)δ: 1.32 (6H, d, J=7.0 Hz), 1.47 (1H, br s), 1.82-2.00 (2H, m), 2.53-2.66 (2H, m), 3.03 (1H, septet, J=6.9 Hz), 3.75 (2H, t, J=6.4 Hz), 7.95 (1H, dd, J=8.6, 2.0 Hz), 8.03 (1H, dd, J=8.6, 1.0 Hz), 8.25 (1H, t, J=0.9 Hz), 8.61 (1H, dd, J=2.0, 1.0 Hz).

### Reference Example 222

To a mixture of 4-[3-(methoxymethoxy)propyl]-3-(1-methylethyl)-1H-pyrazole (1.50 g), 2-chloro-5-nitropyridine (1.23 g) and N,N-dimethylformamide (10 ml) was added sodium hydride (60%, in oil, 0.37 g) at 0° C., and, after termination of hydrogen generation, the mixture was stirred at 80° C. for 3 hours. The reaction mixture was poured into water, and extracted with ethyl acetate. The ethyl acetate layer was washed with saturated aqueous sodium chloride solution, dried (MgSO₄) and concentrated. A mixture of the obtained residue, conc. hydrochloric acid (2 drops) and methanol (6 ml) was stirred overnight at 60° C. The reaction mixture was poured into aqueous sodium hydrogen carbonate, and the precipitated crystals were collected by filtration, washed with water and dried to give 3-[3-(1-methylethyl)-1-(5-nitro-2-pyridyl)-1H-pyrazol-4-yl]-1-propanol (1.60 g, yield 80%) as colorless crystals. melting point: 130-131° C.

¹H-NMR (CDCl₃)δ: 1.34 (6H, d, J=7.0 Hz), 1.36 (1H, t, J=5.0 Hz), 1.84-2.00 (2H, m), 2.55-2.67 (2H, m), 3.04 (1H, septet, J=6.9 Hz), 3.76 (2H, t, J=6.0 Hz), 8.06 (1H, d, J=9.2 Hz), 8.30 (1H, s), 8.51 (1H, dd, J=9.2, 2.8 Hz), 9.20 (1H, dd, J=2.5, 0.7 Hz).

### Reference Example 223

To a mixture of 4-[3-(methoxymethoxy)propyl]-3-(1-methylethyl)-1H-pyrazole (1.52 g), 2-chloro-5-methylpyridine (1.83 g) and N,N-dimethylformamide (15 ml), was added sodium hydride (60%, in oil, 0.43 g) at 0° C., and, after termination of hydrogen generation, the mixture was stirred at 110° C. overnight. The reaction mixture was poured into water, and extracted with ethyl acetate. The ethyl acetate layer was washed with saturated aqueous sodium chloride solution, dried (MgSO₄) and concentrated. A mixture of the obtained residue, conc. hydrochloric acid (2 ml) and methanol (20 ml) was refluxed of 2 hours. The reaction mixture was poured into water, and extracted with ethyl acetate. The ethyl acetate layer was washed with saturated aqueous sodium chloride solution, dried (MgSO₄) and concentrated. The residue was subjected to silica gel column chromatography, and 3-[3-(1-methylethyl)-1-(5-methyl-2-pyridyl)-1H-pyrazol-4-yl]-1-propanol (0.80 g, yield 43%) was obtained as colorless crystals from a fraction eluted with ethyl acetate-hexane (2:3, volume ratio). melting point: 82-83° C.

¹H-NMR (CDCl₃)δ: 1.33 (6H, d, J=7.0 Hz), 1.56 (1H, br s), 1.82-1.97 (2H, m), 2.32 (3H, s), 2.58 (2H, t, J=7.7 Hz), 3.03 (1H, septet, J=7.0 Hz), 3.74 (2H, t, J=6.4 Hz), 7.52-7.60 (1H, m), 7.82 (1H, d, J=8.4 Hz), 8.14-8.16 (1H, m), 8.20 (1H, s).

### Reference Example 224

To a mixture of 3-[3-(1-methylethyl)-1-(5-nitro-2-pyridyl)-1H-pyrazol-4-yl]-1-propanol (1.18 g), methyl (3-methoxy-2-hydroxyphenyl)acetate (800 mg), tributylphosphine (1.64 g) and tetrahydrofuran (40 ml) was added 1,1′-azodicarbonyldipiperidine (2.05 g) at room temperature, and the mixture was stirred overnight. The reaction solution was concentrated. The residue was subjected to silica gel column chromatography, and methyl (3-methoxy-2-{3-[3-(1-methylethyl)-1-(5-nitro-2-pyridyl)-1H-pyrazol-4-yl]propoxy}phenyl)acetate (1.30 g, yield 50%) was obtained as yellow crystals from a fraction eluted with ethyl acetate-hexane (1:4, volume ratio). melting point: 108-109° C.

¹H-NMR (CDCl₃)δ: 1.35 (6H, d, J-7.0 Hz), 2.00-2.17 (2H, m), 2.71 (2H, t, J=7.7 Hz), 3.07 (1H, septet, J=6.9 Hz), 3.68 (3H, s), 3.85 (2H, s), 4.07 (2H, t, J=6.2 Hz), 6.80-6.90 (2H, m), 7.02 (1H, dd, J=8.4, 7.4 Hz), 8.06 (1H, d, J=9.2 Hz), 8.35 (1H, s), 8.51 (1H, dd, J=9.1, 2.5 Hz), 9.20 (1H, d, J=2.2 Hz).

### Reference Example 225

A mixture of methyl (3-methoxy-2-{3-[3-(1-methylethyl)-1-(5-nitro-2-pyridyl)-1H-pyrazol-4-yl]propoxy}phenyl)acetate (0.88 g), 5% palladium-carbon (0.1 g), methanol (4 ml) and tetrahydrofuran (4 ml) was stirred overnight at room temperature under a hydrogen atmosphere. Palladium-carbon was removed by filtration and the filtrate was concentrated. The residue was subjected to silica gel column chromatography, and methyl (2-{3-[1-(5-amino-2-pyridyl)-3-(1-methylethyl)-1H-pyrazol-4-yl]propoxy}-3-methoxyphenyl)acetate (0.80 g, yield 95%) was obtained as a yellow oily substance from a fraction eluted with ethyl acetate-hexane (1:4, volume ratio).

¹H-NMR (CDCl₃)δ: 1.33 (6H, d, J=7.0 Hz), 1.97-2.15 (2H, m), 2.68 (2H, t, J=7.8 Hz), 3.05 (1H, septet, J=6.9 Hz), 3.63 (2H, br s), 3.66 (3H, s), 3.68 (2H, s), 3.83 (3H, s), 4.06 (2H, t, J=6.4 Hz), 6.78-6.88 (2H, m), 6.95-7.27 (2H, m), 7.72 (1H, d, J=8.8 Hz), 7.83 (1H, d, J=2.6 Hz), 8.14 (1H, s).

### Reference Example 226

To a mixture of 4-[3-(methoxymethoxy)propyl]-3-(1-methylethyl)-1H-pyrazole (1.00 g), 3-chloro-6-(trifluoromethyl)pyridazine (1.03 g) and N,N-dimethylformamide (15 ml) was added sodium hydride (60%, in oil, 0.28 g) at 0° C., and, after termination of hydrogen generation, the mixture was stirred at room temperature for 3 hours. The reaction mixture was poured into water, and the precipitated crystals were collected by filtration and washed with water. A mixture of the obtained residue, conc. hydrochloric acid (3 drops) and methanol (15 ml) was refluxed for 4 hours. The reaction mixture was poured into ice water, and the precipitated crystals were collected by filtration, washed with water, dried and subjected to silica gel column chromatography, and 3-{3-(1-methylethyl)-1-[6-(trifluoromethyl)pyridazin-3-yl]-1H-pyrazol-4-yl}-1-propanol (1.00 g, yield 68%) was obtained as colorless crystals from a fraction eluted with ethyl acetate-chloroform (1:3, volume ratio). melting point: 113-114° C.

H-NMR (CDCl₃)δ: 1.33 (6H, d, J=6.6 Hz), 1.42 (1H, t, J=5.1 Hz), 1.84-2.01 (2H, m), 2.63 (2H, t, J=7.9 Hz), 3.05 (1H, septet, J=6.8 Hz), 3.77 (2H, q, J-5.7 Hz), 7.83 (1H, d, J=9.0 Hz), 8.29 (1H, d, J=9.0 Hz), 8.50 (1H, s).

### Reference Example 227

To a mixture of 4-[3-(methoxymethoxy)propyl]-3-(1-methylethyl)-1H-pyrazole (1.00 g), 3-chloro-6-methoxypyridazine (0.82 g) and N,N-dimethylformamide (15 ml) was added sodium hydride (60%, in oil, 0.24 g) at 0° C., and, after termination of hydrogen generation, the mixture was stirred at room temperature for 3 hours. The reaction mixture was poured into water, and the precipitated crystals were collected by filtration and washed with water. A mixture of the obtained wet crystals, conc. hydrochloric acid (3 drops) and methanol (15 ml) was refluxed for 4 hours. The reaction mixture was poured into ice water, and the precipitated crystals were collected by filtration, washed with water, dried and subjected to silica gel column chromatography, and 3-{1-[6-methoxypyridazin-3-yl]-3-(1-methylethyl)-1H-pyrazol-4-yl}-1-propanol (300 mg, yield 23%) was obtained as a colorless oil from a fraction eluted with acetone-chloroform (1:4, volume ratio). melting point: 122-123° C.

¹H-NMR (CDCl₃)δ: 1.32 (6H, d, J=6.9 Hz), 1.39 (1H, t, J=5.3 Hz), 1.84-1.97 (2H, m), 2.60 (2H, t, J=7.7 Hz), 3.03 (1H, septet, J=7.0 Hz), 3.75 (2H, q, J=5.8 Hz), 4.12 (3H, s), 7.06 (1H, d, J=9.3 Hz), 8.11 (1H, d, J=9.3 Hz), 8.32 (1H, s).

### Reference Example 228

To a mixture of 4-[3-(methoxymethoxy)propyl]-3-(1-methylethyl)-1H-pyrazole (1.00 g), 6-chloropyridazine-3-carbonitrile (0.72 g) and N,N-dimethylformamide (15 ml) was added sodium hydride (60%, in oil, 0.24 g) at 0° C., and, after termination of hydrogen generation, the mixture was stirred at room temperature for 3 hours. The reaction mixture was poured into water, and the precipitated crystals were collected by filtration and washed with water. A mixture of the obtained wet crystals, conc. hydrochloric acid (3 drops) and methanol (15 ml) was refluxed for 4 hours. The reaction mixture was poured into ice water, and the precipitated crystals were collected by filtration, washed with water, dried and subjected to silica gel column chromatography, and 6-[4-(3-hydroxypropyl)-3-(1-methylethyl)-1H-pyrazol-1-yl]pyridazine-3-carbonitrile (950 mg, yield 74%) was obtained as colorless crystals from a fraction eluted with ethyl acetate-chloroform (1:2, volume ratio). melting point: 140-141° C.

¹H-NMR (CDCl₃)δ: 1.32 (6H, d, J=7.0 Hz), 1.37 (1H, t, J=5.1 Hz), 1.84-2.01 (2H, m), 2.63 (2H, t, J=7.9 Hz), 3.05 (1H, septet, J=6.9 Hz), 3.77 (2H, q, J=5.6 Hz), 7.82 (1H, d, J=9.0 Hz), 8.25 (1H, d, J=9.0 Hz), 8.48-8.50 (1H, m).

### Reference Example 229

To a mixture of 3-(1-ethylpropyl)-4-[3-(methoxymethoxy)propyl]-1H-pyrazole (2.20 g), 3-chloro-6-(trifluoromethyl)pyridazine (2.17 g) and N,N-dimethylformamide (30 ml) was added sodium hydride (60%, in oil, 0.48 g) at 0° C., and, after termination of hydrogen generation, the mixture was stirred at room temperature for 2 hours. The reaction mixture was poured into water, and extracted with ethyl acetate. The ethyl acetate layer was washed with saturated aqueous sodium chloride solution, dried (MgSO₄) and concentrated. A mixture of the obtained wet crystals, conc. hydrochloric acid (3 drops) and methanol (50 ml) was refluxed for 4 hours. The reaction mixture was poured into water, and extracted with ethyl acetate. The ethyl acetate layer was washed with saturated aqueous sodium chloride solution, dried (MgSO₄) and concentrated. The residue was subjected to silica gel column chromatography, and 3-{3-(1-ethylpropyl)-1-[6-(trifluoromethyl)pyridazin-3-yl]-1H-pyrazol-4-yl}-1-propanol (1.73 g, yield 55%) was obtained as colorless crystals from a fraction eluted with ethyl acetate-hexane (1:2, volume ratio). melting point: 86-87° C.

¹H-NMR (CDCl₃)δ: 0.87 (6H, d, J=7.3 Hz), 1.46 (1H, br s), 1.60-2.00 (6H, m), 2.53-2.70 (3H, m), 3.76 (2H, t, J=6.4 Hz), 7.83 (1H, d, J=9.2 Hz), 8.29 (1H, d, J=9.2 Hz), 8.51 (1H, s).

### Reference Example 230

To a mixture of 4-[3-(methoxymethoxy)propyl]-3-(1-methylethyl)-1H-pyrazole (1.50 g), 2-methylthio-5-(trifluoromethyl)pyrimidine (1.40 g) and N,N-dimethylformamide (15 ml) was added sodium hydride (60%, in oil, 0.48 g) at 0° C., and, after termination of hydrogen generation, the mixture was stirred at room temperature for 4 hours. The reaction mixture was poured into water, and extracted with ethyl acetate. The ethyl acetate layer was washed with saturated aqueous sodium chloride solution, dried (MgSO₄) and concentrated. A mixture of the obtained residue, conc. hydrochloric acid (3 drops) and methanol (50 ml) was refluxed for 4 hours. The reaction mixture was poured into water, and extracted with ethyl acetate. The ethyl acetate layer was washed with saturated aqueous sodium chloride solution, dried (MgSO₄) and concentrated. The residue was subjected to silica gel column chromatography, and 3-{3-(1-methylethyl)-1-[5-(trifluoromethyl)pyrimidin-2-yl]-1H-pyrazol-4-yl}-1-propanol (240 mg, yield 11%) was obtained as colorless crystals from a fraction eluted with ethyl acetate-chloroform (1:3, volume ratio). melting point: 98-99° C.

¹H-NMR (CDCl₃)δ: 1.38 (6H, d, J=7.0 Hz), 1.85-2.01 (2H, m), 2.63 (2H, t, J=7.7 Hz), 3.11 (1H, septet, J=7.0 Hz), 3.77 (2H, t, J=6.2 Hz), 8.34 (1H, s), 8.91 (2H, s).

### Reference Example 231

To a mixture of 4-[3-(methoxymethoxy)propyl]-3-(1-methylethyl)-1H-pyrazole (1.00 g), 2-methylthiopyrimidine-5-carbonitrile (0.80 g) and N,N-dimethylformamide (15 ml) was added sodium hydride (60%, in oil, 0.24 g) at 0° C., and, after termination of hydrogen generation, the mixture was stirred at room temperature for 2 hours. The reaction mixture was poured into water, and extracted with ethyl acetate. The ethyl acetate layer was washed with saturated aqueous sodium chloride solution, dried (MgSO₄) and concentrated. A mixture of the obtained residue, conc. hydrochloric acid (3 drops) and methanol (20 ml) was refluxed for 4 hours. The reaction mixture was poured into water, and extracted with ethyl acetate. The ethyl acetate layer was washed with saturated aqueous sodium chloride solution, dried (MgSO₄) and concentrated. The residue was subjected to silica gel column chromatography, and 2-[4-(3-hydroxypropyl)-3-(1-methylethyl)-1H-pyrazol-1-yl]pyrimidine-5-carbonitrile (450 mg, yield 36%) was obtained as colorless crystals from a fraction eluted with ethyl acetate-chloroform (1:4, volume ratio). melting point: 153-154° C.

¹H-NMR (CDCl₃)δ: 1.38 (6H, d, J=7.0 Hz), 1.44 (1H, t, J=5.2 Hz), 1.84-2.00 (2H, m), 2.62 (2H, t, J=7.8 Hz), 3.10 (1H, septet, J=7.0 Hz), 3.77 (2H, q, J=5.9 Hz), 8.31 (1H, s), 8.93 (2H, s).

### Reference Example 232

To a mixture of 4-[3-(methoxymethoxy)propyl]-3-(1-methylethyl)-1H-pyrazole (1.20 g), 2-chloro-5-ethylpyrimidine (0.89 g) and N,N-dimethylformamide (15 ml) was added sodium hydride (60%, in oil, 0.29 g) at 0° C., and, after termination of hydrogen generation, the mixture was stirred at room temperature for 3 hours. The reaction mixture was poured into water, and extracted with ethyl acetate. The ethyl acetate layer was washed with saturated aqueous sodium chloride solution, dried (MgSO₄) and concentrated. A mixture of the obtained residue, conc. hydrochloric acid (1 ml) and methanol (20 ml) was refluxed for 5 hours. The reaction mixture was poured into water, and extracted with ethyl acetate. The ethyl acetate layer was washed with saturated aqueous sodium chloride solution, dried (MgSO₄) and concentrated. The residue was subjected to silica gel column chromatography, and 3-[1-(5-ethylpyrimidin-2-yl)-3-(1-methylethyl)-1H-pyrazol-4-yl]-1-propanol (1.36 g, yield 88%) was obtained as colorless crystals from a fraction eluted with ethyl acetate-hexane (7:3, volume ratio). melting point: 70-71° C.

¹H-NMR (CDCl₃)δ: 1.27 (3H, t, J=7.5 Hz), 1.37 (6H, d, J=6.8 Hz), 1.73 (1H, br s), 1.83-2.00 (2H, m), 2.54-2.72 (4H, m), 3.11 (1H, septet, J=7.0 Hz), 3.75 (2H, t, J=6.4 Hz), 8.28 (1H, s), 8.53 (2H, s).

### Reference Example 233

To a mixture of 3-(1-ethylpropyl)-4-[3-(methoxymethoxy)propyl]-1H-pyrazole (2.70 g), 2-methylthio-5-(trifluoromethyl)pyrimidine (2.62 g) and tetrahydrofuran (50 ml) was added sodium hydride (60%, in oil, 0.58 g) at 0° C., and, after termination of hydrogen generation, the mixture was stirred at room temperature overnight. The reaction mixture was poured into water, and extracted with ethyl acetate. The ethyl acetate layer was washed with saturated aqueous sodium chloride solution, dried (MgSO₄) and concentrated. A mixture of the obtained residue, conc. hydrochloric acid (3 drops) and methanol (50 ml) was refluxed for 6 hours. The reaction mixture was poured into water, and extracted with ethyl acetate. The ethyl acetate layer was washed with saturated aqueous sodium chloride solution, dried (MgSO₄) and concentrated. The residue was subjected to silica gel column chromatography, and 3-{3-(1-ethylpropyl)-1-[5-(trifluoromethyl)pyrimidin-2-yl]-1H-pyrazol-4-yl}-1-propanol (1.19 g, yield 31%) was obtained as a yellow oily substance from a fraction eluted with ethyl acetate-hexane (2:3, volume ratio).

¹H-NMR (CDCl₃)δ: 0.87 (6H, t, J=7.3 Hz), 1.63-2.00 (6H, m), 2.55-2.80 (3H, m), 3.76 (2H, t, J=6.2 Hz), 8.35 (1H, s), 8.92 (2H, s).

### Reference Example 234

A mixture of methyl (2-hydroxy-3-methoxyphenyl)acetate (1.00 g), benzyl alcohol (1.10 g), p-toluenesulfonic acid-monohydrate (0.10 g) and toluene (15 ml) was stirred overnight at 90° C. while evaporating produced methanol. The reaction mixture was concentrated. The residue was subjected to silica gel column chromatography, and benzyl (2-hydroxy-3-methoxyphenyl)acetate (1.35 g, yield 97%) was obtained as a colorless oil from a fraction eluted with ethyl acetate-hexane (1:2, volume ratio).

¹H-NMR (CDCl₃)δ: 3.73 (2H, s), 3.88 (3H, s), 5.16 (2H, s), 5.87 (1H, s), 6.80 (3H, s), 7.28-7.40 (5H, m).

### Reference Example 235

A mixture of ethyl 3-[3-(1-ethylpropyl)-1H-pyrazol-4-yl]propanoate (10.57 g), benzyl bromide (4.40 ml), potassium carbonate (5.00 g) and N,N-dimethylformamide (80 ml) was stirred at 70° C. for 6 hours. The reaction mixture was poured into water, and extracted with ethyl acetate. The ethyl acetate layer was washed with saturated aqueous sodium chloride solution, dried (MgSO₄) and concentrated. The residue was subjected to silica gel column chromatography, and a yellow oily substance was obtained from a fraction eluted with ethyl acetate-hexane (1:4, volume ratio). To a mixture of lithium aluminum hydride (1.50 g) and tetrahydrofuran (20 ml) was slowly added a solution of the above-mentioned oily substance in tetrahydrofuran (10 ml) at 0° C., and the mixture was stirred at room temperature for 30 minutes. Acetone (10 ml) was slowly added to decompose excess lithium aluminum hydride, and brine (4 ml) was further added. The precipitate was removed by filtration and the filtrate was concentrated. The residue was subjected to silica gel column chromatography, and 3-[1-benzyl-3-(1-ethylpropyl)-1H-pyrazol-4-yl]-1-propanol (7.69 g, yield 80%) was obtained as a colorless oil from a fraction eluted with acetone-hexane (1:2, volume ratio).

¹H-NMR (CDCl₃)δ: 0.83 (6H, t, J=7.3 Hz), 1.35 (1H, t, J=5.4 Hz), 1.60-1.85 (6H, m), 2.40-2.65 (3H, m), 3.67 (2H, q, J=5.9 Hz), 5.24 (2H, s), 7.03-7.40 (6H, m).

### Reference Example 236

To a mixture of 3-[1-benzyl-3-(1-ethylpropyl)-1H-pyrazol-4-yl]-1-propanol (7.53 g), N-ethyldiisopropylamine (11.5 ml) and tetrahydrofuran (100 ml) was added chloromethyl methyl ether (6.40 ml) at 0° C. and the mixture was stirred at room temperature overnight. The reaction mixture was poured into water, and extracted with ethyl acetate. The ethyl acetate layer was washed with saturated aqueous sodium chloride solution, dried (MgSO₄) and concentrated. The residue was subjected to silica gel column chromatography, and a colorless oil was obtained from a fraction eluted with ethyl acetate-hexane (1:2, volume ratio). A mixture of the obtained oily substance, 5% palladium-carbon (0.8 g) and tetrahydrofuran (50 ml) was stirred overnight at 50° C. under a hydrogen atmosphere. Palladium-carbon was removed by filtration and the filtrate was concentrated. The residue was subjected to silica gel column chromatography, and a colorless oil was obtained from a fraction eluted with acetone-hexane (2:3, volume ratio). 3-(1-Ethylpropyl)-4-[3-(methoxymethoxy)propyl]-1H-pyrazole (4.93 g, yield 77%) was obtained as a colorless oil.

¹H-NMR (CDCl₃)δ: 0.82 (6H, d, J=7.3 Hz), 1.50-1.94 (6H, m), 2.44-2.70 (3H, m), 3.38 (3H, s), 3.57 (2H, t, J=6.4 Hz), 4.64 (2H, s), 7.36 (1H, s).

### Reference Example 237

A mixture of 3-ethylsalicylaldehyde (8.10 g), benzyl bromide (11.07 g), potassium carbonate (8.94 g) and N,N-dimethylformamide (30 ml) was stirred at 50° C. for 1 hour. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and 2-benzyloxy-3-ethylbenzaldehyde (12.50 g, yield 96%) was obtained as a colorless oil from a fraction eluted with ethyl acetate-hexane (2:98, volume ratio).

¹H-NMR (CDCl₃)δ: 1.27 (3H, t, J=7.6 Hz), 2.76 (2H, q, J=7.6 Hz), 4.98 (2H, s), 7.22 (1H, t, J=7.6 Hz), 7.39-7.43 (5H, m), 7.51-7.53 (1H, m), 7.70-7.72 (1H, m), 10.28 (1H, m).

### Reference Example 238

A mixture of ethyl 3-(3-ethoxy-1H-pyrazol-4-yl)propanoate (7.01 g), sodium hydride (60%, in oil, 1.59 g) and N,N-dimethylformamide (165 ml) was stirred at room temperature for 30 minutes. 2-Chloro-4-(trifluoromethyl)pyridine (6.00 g) was added and the mixture was stirred overnight. Saturated aqueous ammonium chloride solution was added to the reaction mixture, and extracted with ethyl acetate. The ethyl acetate layer was washed with saturated aqueous sodium chloride solution, dried (MgSO₄) and concentrated. The residue was subjected to silica gel column chromatography, and ethyl 3-{3-ethoxy-1-[4-(trifluoromethyl)-2-pyridinyl]-1H-pyrazol-4-yl}propanoate (9.05 g, yield 77%) was obtained as a pale-yellow oily substance from a fraction eluted with ethyl acetate-hexane (1:9, volume ratio).

¹H-NMR (CDCl₃)δ: 1.26 (3H, t, J=7.0 Hz), 1.44 (3H, t, J=7.0 Hz), 2.56-2.66 (2H, m), 2.70-2.81 (2H, m), 4.15 (2H, q, J=7.0 Hz), 4.37 (2H, q, J=7.0 Hz), 7.18-7.24 (1H, m), 7.91-7.94 (1H, m), 8.18 (1H, s), 8.45 (1H, d, J=5.0 Hz).

### Reference Example 239

To a solution of ethyl 3-{3-ethoxy-1-[4-(trifluoromethyl)-2-pyridinyl]-1H-pyrazol-4-yl}propanoate (10.2 g) in tetrahydrofuran (280 ml) was dropwise added a 0.93 M solution (92.0 ml) of diisobutylaluminum hydride in hexane at 0° C., and the mixture was stirred at room temperature for 1 hour. 1N Hydrochloric acid was added to the reaction mixture and extracted with ethyl acetate. The ethyl acetate layer was washed with saturated aqueous sodium chloride solution, dried (MgSO₄) and concentrated to give 3-{3-ethoxy-1-[4-(trifluoromethyl)-2-pyridinyl]-1H-pyrazol-4-yl}-1-propanol (9.07 g, quantitative) as a white solid. The crystals were recrystallized from ethyl acetate-hexane to give colorless crystals. melting point: 73-74° C.

### Reference Example 240

A mixture of ethyl 3-isopropyl-1H-pyrazole-4-carboxylate (12.8 g), sodium hydride (60%, in oil, 3.08 g) and N,N-dimethylformamide (350 ml) was stirred at room temperature for 30 minutes. 2,5-Dichloropyridine (11.4 g) was added and the mixture was stirred overnight at 100° C. Saturated aqueous ammonium chloride solution was added to the reaction mixture, and extracted with ethyl acetate. The ethyl acetate layer was washed with saturated aqueous sodium chloride solution and saturated aqueous sodium chloride solution, dried (MgSO₄) and concentrated. The residue was subjected to silica gel column chromatography, and a white solid was obtained from a fraction eluted with ethyl acetate-hexane (1:19, volume ratio). To a solution of the obtained solid in tetrahydrofuran (230 ml) was dropwise added a 1.0 M solution (176 ml) of diisobutylaluminum hydride in hexane at 0° C., and the mixture was stirred at room temperature for 1 hour. Dilute hydrochloric acid was added to the reaction mixture, and extracted with ethyl acetate. The ethyl acetate layer was washed with saturated aqueous sodium chloride solution, dried (MgSO₄) and concentrated. The residue was subjected to silica gel column chromatography, and [1-(5-chloro-2-pyridinyl)-3-isopropyl-1H-pyrazol-4-yl]methanol (12.6 g, yield 71%) was obtained as a white solid from a fraction eluted with ethyl acetate-hexane (1:4, volume ratio). The crystals were recrystallized from ethyl acetate-hexane to give colorless crystals. melting point: 135-136° C.

### Reference Example 241

A mixture of ethyl (E)-3-[1-(5-chloro-2-pyridinyl)-3-isopropyl-1H-pyrazol-4-yl]propenoate (1.35 g), platinum oxide (100 mg) and ethanol (100 ml) was stirred at room temperature for 1 hour under a hydrogen atmosphere. Platinum oxide was removed by filtration and the filtrate was concentrated. The residue was subjected to silica gel column chromatography, and ethyl 3-[1-(5-chloro-2-pyridinyl)-3-isopropyl-1H-pyrazol-4-yl]propanoate (1.09 g, yield 68%) was obtained as a white solid from a fraction eluted with ethyl acetate-hexane (1:19, volume ratio). The crystals were recrystallized from ethyl acetate-hexane to give colorless crystals. melting point: 70-71° C.

### Reference Example 242

To a solution of ethyl 3-[1-(5-chloro-2-pyridinyl)-3-isopropyl-1H-pyrazol-4-yl]propanoate (1.08 g) in tetrahydrofuran (30 ml) was dropwise added a 0.93 M a solution (9.8 ml) of diisobutylaluminum hydride in hexane at 0° C., and the mixture was stirred at room temperature for 1 hour. 1N Hydrochloric acid was added to the reaction mixture, and extracted with ethyl acetate. The ethyl acetate layer was washed with saturated aqueous sodium chloride solution, dried (MgSO₄) and concentrated to give 3-[1-(5-chloro-2-pyridinyl)-3-isopropyl-1H-pyrazol-4-yl]-1-propanol (0.92 g, quantitative) as a white solid. The crystals were recrystallized from ethyl acetate-hexane to give colorless crystals. melting point: 93-95° C.

### Reference Example 243

Diethyl ethoxymethylenemalonate (56.9 ml) was added to a solution of ethylhydrazine oxalate (42.6 g) in toluene (150 ml)-acetic acid (150 ml)-water (100 ml) and the mixture was stirred at room temperature for 1 hour, and at 100° C. overnight. The reaction solution was cooled to room temperature, the organic solvent was evaporated under reduced pressure, and the residue was extracted with ethyl acetate. The ethyl acetate layer was washed with saturated aqueous sodium chloride solution, dried (MgSO₄) and concentrated. The residue was washed with diisopropyl ether to give a pale-yellow solid. A mixture of the obtained solid, benzyl bromide (29.0 ml), potassium carbonate (33.7 g) and N,N-dimethylformamide (350 ml) was stirred at room temperature for 2.5 days and saturated aqueous ammonium chloride solution was added to the reaction mixture. The mixture was extracted with ethyl acetate. The ethyl acetate layer was washed with saturated aqueous sodium chloride solution, dried (MgSO₄) and concentrated. The residue was subjected to silica gel column chromatography, and ethyl 3-benzyloxy-1-ethyl-1H-pyrazole-4-carboxylate (34.0 g, yield 43%) was obtained as a pale yellow oily substance from a fraction eluted with ethyl acetate-hexane (1:4, volume ratio).

¹H-NMR (CDCl₃)δ: 1.33 (3H, t, J=7.4 Hz), 1.46 (3H, t, J=7.4 Hz), 4.01 (2H, q, J=7.4 Hz), 4.27 (2H, q, J=7.4 Hz), 5.34 (2H, s), 7.22-7.42 (3H, m), 7.46-7.54 (2H, m), 7.72 (1H, s).

### Reference Example 244

To a mixture of ethyl 3-benzyloxy-1-ethyl-1H-pyrazole-4-carboxylate (34.0 g) and tetrahydrofuran (500 ml) was slowly added lithium aluminum hydride (4.70 g) at 0° C. and the mixture was stirred at room temperature for 1.5 hours. 1N Hydrochloric acid was added to the reaction mixture, and extracted with ethyl acetate. The ethyl acetate layer was washed with saturated aqueous sodium chloride solution, dried (MgSO₄) and concentrated. The residue was subjected to silica gel column chromatography, and (3-benzyloxy-1-ethyl-1H-pyrazol-4-yl)methanol (19.9 g, yield 69%) was obtained as a colorless oil from a fraction eluted with ethyl acetate-hexane (3:2, volume ratio).

¹H-NMR (CDCl₃)δ: 1.42 (3H, t, J=7.2 Hz), 3.98 (2H, d, J=7.2 Hz), 4.47 (2H, s), 5.24 (2H, s), 7.20 (1H, s), 7.27-7.39 (3H, m), 7.40-7.46 (2H, m).

### Reference Example 245

To a mixture of (3-benzyloxy-1-ethyl-1H-pyrazol-4-yl)methanol (1.40 g), acetone cyanohydrin (1.10 ml), tributylphosphine (3.00 ml) and tetrahydrofuran (60 ml) was added 1,1′-azodicarbonyldipiperidine (3.04 g) at room temperature, and the mixture was stirred overnight. The reaction solution was concentrated. The residue was subjected to silica gel column chromatography, and (3-benzyloxy-1-ethyl-1H-pyrazol-4-yl)acetonitrile (0.72 g, yield 49%) was obtained as a yellow oily substance from a fraction eluted with ethyl acetate-hexane (1:5, volume ratio).

¹H-NMR (CDCl₃)δ: 1.44 (3H, t, J=7.2 Hz), 3.43 (2H, s), 3.99 (2H, q, J=7.2 Hz), 5.22 (2H, s), 7.23-7.46 (6H, m).

### Reference Example 246

A mixture of (3-benzyloxy-1-ethyl-1H-pyrazol-4-yl)acetonitrile (720 mg), 6N aqueous sodium hydroxide solution (20 ml), tetrahydrofuran (20 ml) and ethanol (20 ml) was stirred under reflux for 2 days. After cooling, the reaction mixture was acidified by adding 1N hydrochloric acid, and extracted with ethyl acetate. The ethyl acetate layer was washed with saturated aqueous sodium chloride solution, dried (MgSO₄) and concentrated. A mixture of the residue, a 10% solution (30 ml) of hydrochloric acid in methanol and methanol (30 ml) was stirred at room temperature for 2.5 hours. After concentration, the residue was subjected to silica gel column chromatography, and methyl (3-benzyloxy-1-ethyl-1H-pyrazol-4-yl)acetate (470 mg, yield 57%) was obtained as a yellow oily substance from a fraction eluted with ethyl acetate-hexane (2:3, volume ratio).

¹H-NMR (CDCl₃)δ: 1.43 (3H, t, J=7.2 Hz), 3.40 (2H, s), 3.68 (3H, s), 3.98 (2H, q, J=7.2 Hz), 5.22 (2H, s), 7.23 (1H, s), 7.27-7.39 (3H, m), 7.40-7.47 (2H, m).

### Reference Example 247

A mixture of methyl (3-benzyloxy-1-ethyl-1H-pyrazol-4-yl)acetate (11.0 g), 5% palladium-carbon (2.19 g) and ethanol (300 ml) was stirred overnight at room temperature under a hydrogen atmosphere. Palladium-carbon was removed by filtration and the filtrate was concentrated. The residue was subjected to silica gel column chromatography, and methyl (1-ethyl-3-hydroxy-1H-pyrazol-4-yl)acetate (7.17 g, yield 97%) was obtained as a white solid from a fraction eluted with ethyl acetate. The crystals were recrystallized from ethyl acetate-hexane to give colorless crystals. melting point: 72-73° C.

### Reference Example 248

To a solution of cyclohexylhydrazine hydrochloride (30.0 g) in toluene (100 ml)-acetic acid (100 ml) was added sodium acetate (16.3 g) and the mixture was reacted at room temperature for 10 minutes. A solution of diethyl ethoxymethylenemalononate (39.8 ml) was added and the mixture was stirred overnight at 80° C. After cooling the reaction solution to room temperature, the resulting precipitate was removed by filtration. The filtrate was concentrated. The residue was subjected to silica gel column chromatography, and ethyl 1-cyclohexyl-3-hydroxy-1H-pyrazole-4-carboxylate (46.2 g, yield 97%) was obtained as a purple solid from a fraction eluted with ethyl acetate-hexane (1:4, volume ratio). The crystals were recrystallized from ethyl acetate-hexane to give colorless crystals. melting point: 91-92° C.

### Reference Example 249

A mixture of ethyl 1-cyclohexyl-3-hydroxy-1H-pyrazole-4-carboxylate (46.0 g), benzyl bromide (24.1 ml), potassium carbonate (28.1 g) and N,N-dimethylformamide (400 ml) was stirred overnight at room temperature. Saturated aqueous ammonium chloride solution was added to the reaction mixture, and extracted with ethyl acetate. The ethyl acetate layer was washed with saturated aqueous ammonium chloride solution and saturated aqueous sodium chloride solution, dried (MgSO₄) and concentrated. The residue was subjected to silica gel column chromatography, and ethyl 3-benzyloxy-1-cyclohexyl-1H-pyrazole-4-carboxylate (61.5 g, yield 97%) was obtained as a yellow oily substance from a fraction eluted with ethyl acetate-hexane (1:19, volume ratio).

¹H-NMR (CDCl₃)δ: 1.10-1.28 (3H, m), 1.37 (3H, t, J=7.2 Hz), 1.38-1.49 (2H, m), 1.56-1.82 (5H, m), 3.81-3.92 (1H, m), 4.31 (2H, q, J=7.2 Hz), 5.41 (2H, s), 7.32-7.39 (5H, m), 7.77 (1H, s).

### Reference Example 250

To a mixture of ethyl 3-benzyloxy-1-cyclohexyl-1H-pyrazole-4-carboxylate (31.5 g) and tetrahydrofuran (300 ml) was slowly added lithium aluminum hydride (2.73 g) at 0° C. and the mixture was stirred at room temperature for 1.5 hours. Aluminum lithium hydride (1.81 g) was added, and the mixture was stirred at room temperature for 1 hour. 1N Hydrochloric acid was added to the reaction mixture, and extracted with ethyl acetate. The ethyl acetate layer was washed with 1N hydrochloric acid and saturated aqueous sodium chloride solution, dried (MgSO₄) and concentrated. The residue was subjected to silica gel column chromatography, and (3-benzyloxy-1-cyclohexyl-1H-pyrazol-4-yl)methanol (16.5 g, yield 60%) was obtained as a colorless oil from a fraction eluted with ethyl acetate-hexane (1:1, volume ratio).

¹H-NMR (CDCl₃)δ: 1.14-1.35 (3H, m), 1.40-1.86 (1H, brm), 1.59-1.86 (7H, m), 3.87-4.00 (1H, m), 4.48 (2H, d, J=4.5 Hz), 5.24 (2H, s), 7.31-7.41 (6H, m).

### Reference Example 251

To a mixture of (3-benzyloxy-1-cyclohexyl-1H-pyrazol-4-yl)methanol (16.5 g), acetone cyanohydrin (8.77 ml), tributylphosphine (21.5 ml) and tetrahydrofuran (350 ml) was added a 40% solution (39.1 ml) of diethyl azodicarboxylate in toluene at room temperature and the mixture was stirred overnight. The reaction solution was concentrated and diisopropyl ether was added to the residue. The resulting unnecessary material was removed by filtration. The filtrate was concentrated. The residue was subjected to silica gel column chromatography, and a pale yellow oily substance was obtained from a fraction eluted with ethyl acetate-hexane (1:5, volume ratio). A mixture of the obtained oily substance, 6N aqueous sodium hydroxide solution (100 ml), tetrahydrofuran (100 ml) and ethanol (100 ml) was stirred under reflux for one day. After cooling to room temperature, the reaction solution was concentrated. The residue was diluted with water (300 ml) and washed with ethyl acetate. The aqueous layer was acidified by adding conc. hydrochloric acid, and extracted with ethyl acetate. The ethyl acetate layer was washed with saturated aqueous sodium chloride solution, dried (MgSO₄) and concentrated to give (3-benzyloxy-1-cyclohexyl-1H-pyrazol-4-yl)acetic acid (7.86 g, yield 44%) as a yellow oily substance.

¹H-NMR (CDCl₃)δ: 1.14-1.28 (3H, m), 1.54-1.84 (7H, m), 3.40 (2H, s), 3.76-3.92 (1H, m), 5.05 (2H, s), 7.32-7.41 (6H, m)

### Reference Example 252

A mixture of (3-benzyloxy-1-cyclohexyl-1H-pyrazol-4-yl)acetic acid (7.86 g), a 10% solution (125 ml) of hydrochloric acid in methanol and methanol (125 ml) was stirred overnight at room temperature. The reaction solution was concentrated and the residue was diluted with ethyl acetate. The diluted solution was washed with saturated aqueous sodium chloride solution, dried (MgSO₄) and concentrated. The residue was subjected to silica gel column chromatography, and methyl (3-benzyloxy-1-cyclohexyl-1H-pyrazol-4-yl)acetate (1.98 g, yield 24%) was obtained as a yellow oily substance from a fraction eluted with ethyl acetate-hexane (1:4, volume ratio).

¹H-NMR (CDCl₃)δ: 1.12-1.30 (3H, m), 1.52-1.84 (7H, m), 3.38 (2H, s), 3.70 (3H, s), 3.76-3.89 (1H, m), 5.06 (2H, s), 7.33-7.42 (6H, m).

### Reference Example 253

A mixture of methyl (3-benzyloxy-1-cyclohexyl-1H-pyrazol-4-yl)acetate (1.98 g), 5% palladium-carbon (400 mg) and ethanol (60 ml) was stirred overnight at room temperature under a hydrogen atmosphere. Palladium-carbon was removed by filtration and the filtrate was concentrated. The residue was subjected to silica gel column chromatography, and methyl (1-cyclohexyl-3-hydroxy-1H-pyrazol-4-yl)acetate (1.24 g, yield 92%) was obtained as a white solid from a fraction eluted with ethyl acetate-hexane (3:1, volume ratio). The crystals were recrystallized from ethyl acetate-hexane to give colorless crystals. melting point: 135-136° C.

### Reference Example 254

To a solution of diethyl 2-formylsuccinate (2.02 g) in ethanol (15 ml) was dropwise added a solution of methylhydrazine (580 μL) in ethanol (5 ml) at 0° C. The reaction solution was stirred at 0° C. for 30 minutes and at room temperature for 1 hour, followed by heating to 80° C. After stirring at said temperature overnight, the reaction solution was concentrated. The obtained brown solid was recrystallized from ethyl acetate-hexane to give ethyl (5-hydroxy-1-methyl-1H-pyrazol-4-yl)acetate (1.42 g, yield 77%) as colorless crystals. melting point: 104-105° C.

### Reference Example 255

To a solution of ethylhydrazine oxalate (4.08 g) in ethanol (30 ml) was added sodium ethoxide (3.70 g) at 0° C. The mixture was stirred at room temperature for 1 hour and a solution of diethyl 2-formylsuccinate (5.00 g) in ethanol (30 ml) was dropwise added at 0° C. The reaction solution was stirred at 0° C. for 30 minutes and at room temperature for 2 hours, which was followed by heating until reflux. After stirring at said temperature overnight, the reaction solution was cooled to room temperature, and the resulting precipitate was removed by filtration. The filtrate was concentrated. The obtained residue was subjected to silica gel column chromatography, and ethyl (1-ethyl-5-hydroxy-1H-pyrazol-4-yl)acetate (2.36 g, yield 48%) was obtained as a brown solid from a fraction eluted with ethyl acetate-hexane (1:9, volume ratio). The crystals were recrystallized from ethyl acetate-hexane to give colorless crystals. melting point: 107-108° C.

### Reference Example 256

To a solution of ethyl hydrazinoacetate hydrochloride (3.56 g) in ethanol (25 ml) was added 1N aqueous sodium hydroxide solution (23.1 ml) at 0° C. The reaction solution was stirred at room temperature for 1 hour and a solution of ethyl 2-formylpropanoate (3.00 g) in ethanol (75 ml) was dropwise added at 0° C. The reaction solution was stirred at room temperature for 1 hour, which was followed by heating until reflux. After stirring overnight, the reaction solution was cooled to room temperature, and concentrated. The obtained residue was subjected to silica gel column chromatography, and ethyl (5-hydroxy-4-methyl-1H-pyrazol-1-yl)acetate (3.35 g, yield 79%) was obtained as a colorless oil from a fraction eluted with methanol-ethyl acetate (1:7, volume ratio).

¹H-NMR (CDCl₃)δ: 1.25-1.32 (3H, m), 1.39 (1.0H, d, J=8.1 Hz), 1.89 (2H, s), 3.22 (0.3H, t, J=8.1 Hz), 4.17-4.26 (2H, m), 4.45 (0.6H, s), 4.58 (1.4H, s), 7.22-7.24 (0.7H, m), 7.29-7.31 (0.3H, m).

### Reference Example 257

To a solution of ethyl hydrazinoacetate hydrochloride (1.64 g) in ethanol (10 ml) was dropwise added 1N aqueous sodium hydroxide solution (10.6 ml) at 0° C. The reaction solution was stirred at room temperature for 1 hour and a solution of ethyl 2-formylbutanoate (2.13 g) in ethanol (30 ml) was dropwise added at 0° C. The reaction solution was stirred at room temperature for 2.5 hours, and at 80° C. overnight. The reaction solution was cooled to room temperature and concentrated. The obtained residue was subjected to silica gel column chromatography, and ethyl (4-ethyl-5-hydroxy-1H-pyrazol-1-yl)acetate (1.54 g, yield 81%) was obtained as a white solid from a fraction eluted with ethyl acetate-hexane (19:1, volume ratio). The crystals were recrystallized from ethyl acetate-hexane to give colorless crystals. melting point: 77-78° C.

### Reference Example 258

A mixture of ethyl (E)-3-[1-benzyl-3-(1-methylethyl)-1H-pyrazol-4-yl]propenoate (30.25 g), 5% palladium-carbon (3.5 g) and tetrahydrofuran (200 ml) was stirred overnight at room temperature under a hydrogen atmosphere. Palladium-carbon was removed by filtration and the filtrate was concentrated. The residue was subjected to silica gel column chromatography, and ethyl 3-[1-benzyl-3-(1-methylethyl)-1H-pyrazol-4-yl]propanoate (11.73 g, yield 39%) was obtained as a yellow oily substance from a fraction eluted with ethyl acetate-hexane (1:3, volume ratio).

¹H-NMR (CDCl₃)δ: 1.20 (3H, t, J=7.2 Hz), 1.30 (6H, d, J=7.0 Hz), 2.44-2.55 (2H, m), 2.68-2.79 (2H, m), 2.99 (1H, septet, J=7.0 Hz), 4.09 (2H, q, J=7.2 Hz), 5.23 (2H, s), 7.12-7.40 (6H, m).

### Reference Example 259

Ethyl 3-[3-(1-methylethyl)-1H-pyrazol-4-yl]propanoate (10.06 g, yield 47%) was obtained as a yellow oily substance from a fraction eluted following the compound described in Reference Example 258 in the silica gel column chromatography described in Reference Example 258.

¹H-NMR (CDCl₃)δ: 1.25 (3H, t, J=7.2 Hz), 1.29 (6H, d, J=7.0 Hz), 2.50-2.60 (2H, m), 2.72-2.83 (2H, m), 3.06 (1H, septet, J=7.0 Hz), 4.14 (2H, q, J=7.2 Hz), 7.34 (1H, s).

### Reference Example 260

To a mixture of 2-benzyloxy-3-ethylbenzaldehyde (12.40 g), methyl (methylthio)methyl sulfoxide (12.82 g) and tetrahydrofuran (100 ml) was added a 40% solution (2.00 ml) of benzyltrimethylammonium hydroxide in methanol at room temperature, and the mixture was stirred at 65° C. for 2 hours. The reaction solution was concentrated. The residue was subjected to silica gel column chromatography, and 2-(2-benzyloxy-3-ethylphenyl)-1-(methylthio)vinyl methyl sulfoxide (15.20 g, yield 85%) was obtained as a yellow oily substance from a fraction eluted with ethyl acetate-hexane (1:1, volume ratio).

¹H-NMR (CDCl₃)δ: 1.24 (3H, t, J=7.6 Hz), 2.29 (3H, s), 2.72 (2H, q, J=7.6 Hz), 2.72 (3H, s), 4.79-4.82 (2H, m), 7.16 (1H, t, J=7.6 Hz), 7.29 (1H, dd, J=7.6, 1.6 Hz), 7.32-7.42 (3H, m), 7.49-7.51 (2H, m), 7.95 (1H, dd, J=7.6, 1.6 Hz), 8.03 (1H, s).

### Reference Example 261

A mixture of 2-(2-benzyloxy-3-ethylphenyl)-1-(methylthio)vinyl methyl sulfoxide (14.90 g), a 10% solution (100 ml) of hydrogen chloride in methanol and methanol (100 ml) was refluxed for 16 hours. The reaction solution was concentrated. Ethyl acetate and aqueous sodium hydrogen carbonate were added to the residue and the mixture extracted. The ethyl acetate layer was washed with saturated aqueous sodium chloride solution, dried (MgSO₄) and concentrated. The residue was subjected to silica gel column chromatography, and methyl (2-benzyloxy-3-ethylphenyl)acetate (9.60 g, yield 79%) was obtained as a colorless oil from a fraction eluted with ethyl acetate-hexane (4:96, volume ratio).

¹H-NMR (CDCl₃)δ: 1.25 (3H, t, J=7.6 Hz), 2.73 (2H, q, J=7.6 Hz), 3.66 (3H, s), 3.69 (2H, s), 4.84 (2H, s), 7.08 (1H, t, J=7.6 Hz), 7.13 (1H, dd, J=7.6,1.6 Hz), 7.19 (1H, dd, J=7.6,1.6 Hz), 7.32-7.43 (3H, m), 7.46-7.48 (2H, m).

### Reference Example 262

A mixture of methyl (2-benzyloxy-3-ethylphenyl)acetate (9.20 g), 5% palladium-carbon (1.00 g) and methanol (50 ml) was stirred overnight at room temperature under a hydrogen atmosphere. Palladium-carbon was removed by filtration and the filtrate was concentrated. The residue was subjected to silica gel column chromatography, and methyl (3-ethyl-2-hydroxyphenyl)acetate (5.40 g, yield 86%) was obtained as a colorless oil from a fraction eluted with ethyl acetate-hexane (1:9, volume ratio).

¹H-NMR (CDCl₃)δ: 1.23 (3H, t, J=7.6 Hz), 2.69 (2H, q, J=7.6 Hz), 3.68 (2H, s), 3.75 (3H, s), 6.83 (1H, t, J=7.6 Hz), 6.94 (1H, dd, J=7.6,1.6 Hz), 7.10 (1H, dd, J=7.6,1.2 Hz), 7.53 (1H, s).

### Reference Example 263

A mixture of 2-coumaranone (25.00 g), a 10% solution (30 ml) of hydrogen chloride in methanol and methanol (30 ml) was stirred at 50° C. for 30 minutes. The reaction solution was concentrated. Ethyl acetate and aqueous sodium hydrogen carbonate were added to the residue and the mixture was extracted. The ethyl acetate layer was washed with saturated aqueous sodium chloride solution, dried (MgSO₄) and concentrated. The residue was subjected to silica gel column chromatography, and methyl (2-hydroxyphenyl)acetate (30.60 g, yield 99%) was obtained as a colorless oil from a fraction eluted with diethyl ether.

¹H-NMR (CDCl₃)δ: 3.68 (2H, s), 3.74 (3H, s), 6.86-6.93 (2H, m), 7.10 (1H, dd, J=7.2,1.6 Hz), 7.16-7.20 (1H, m), 7.35 (1H, brs).

### Reference Example 264

To a mixture of methyl (2-hydroxyphenyl)acetate (4.99 g), diisopropylamine (610 mg) and methylene chloride (300 ml) was slowly added N-bromosuccinimide (5.34 g) under ice-cooling, and the mixture was stirred for 1 hour. The reaction mixture was poured into dilute hydrochloric acid, and extracted with chloroform. The chloroform layer was washed with saturated aqueous sodium chloride solution, dried (MgSO₄) and concentrated. The residue was subjected to silica gel column chromatography, and methyl (3-bromo-2-hydroxyphenyl)acetate (5.60 g, yield 76%) was obtained as a colorless oil from a fraction eluted with chloroform.

¹H-NMR (CDCl₃)δ: 3.71 (2H, s), 3.73 (3H, s), 6.32 (1H, s), 6.78 (1H, t, J=8.0 Hz), 7.11 (1H, dt, J=8.0,0.8 Hz), 7.41 (1H, dd, J=8.0, 1.6 Hz).

### Reference Example 265

A mixture of methyl (3-bromo-2-hydroxyphenyl)acetate (4.30 g), benzyl bromide (3.30 g), potassium carbonate (4.84 g) and acetone (50 ml) was refluxed for 1 hour. The reaction solution was concentrated. The residue was subjected to silica gel column chromatography, and methyl (2-benzyloxy-3-bromophenyl)acetate (4.10 g, yield 70%) was obtained as a colorless oil from a fraction eluted with ethyl acetate-hexane (4:96, volume ratio).

¹H-NMR (CDCl₃)δ: 3.65 (3H, s), 3.66 (2H, s), 5.01 (2H, s), 7.00 (1H, t, J=8.0 Hz), 7.23 (1H, dd, J=8.0, 1.2 Hz), 7.33-7.43 (3H, m), 7.49-7.54 (3H, m).

### Reference Example 266

A mixture of methyl (2-benzyloxy-3-bromophenyl)acetate (2.01 g), copper(I) cyanide (2.14 g) and N,N-dimethylformamide (30 ml) was stirred at 190° C. for 16 hours. The reaction mixture was poured into a mixture of iron(III) chloride and dilute hydrochloric acid. The mixture was stirred for 1 hour and extracted with ethyl acetate. The ethyl acetate layer was washed with saturated aqueous sodium chloride solution, dried (MgSO₄) and concentrated. The residue was subjected to silica gel column chromatography, and methyl (2-benzyloxy-3-cyanophenyl)acetate (1.20 g, yield 71%) was obtained as a colorless oil from a fraction eluted with ethyl acetate-hexane (1:4, volume ratio).

¹H-NMR (CDCl₃)δ: 3.62 (2H, s), 3.64 (3H, s), 5.24 (2H, s), 7.16 (1H, t, J=7.6 Hz), 7.34-7.42 (3H, m), 7.46-7.50 (3H, m), 7.57 (1H, dd, J=7.6, 1.6 Hz).

### Reference Example 267

A mixture of methyl (2-benzyloxy-3-cyanophenyl)acetate (1.10 g), 5% palladium-carbon (110 mg) and methanol (15 ml) was stirred overnight at room temperature under a hydrogen atmosphere. Palladium-carbon was removed by filtration and the filtrate was concentrated. The residue was subjected to silica gel column chromatography, and methyl (3-cyano-2-hydroxyphenyl)acetate (700 mg, yield 94%) was obtained as a colorless oil from a fraction eluted with ethyl acetate-hexane (3:7, volume ratio).

¹H-NMR (CDCl₃)δ: 3.73 (2H, s), 3.80 (3H, s), 6.95 (1H, t, J=7.6 Hz), 7.31 (1H, dt, J=7.6, 0.8 Hz), 7.48 (1H, dd, J=7.6, 1.6 Hz).

### Reference Example 268

A mixture of methyl (2-benzyloxy-3-bromophenyl)acetate 1.90 g), copper(I) chloride (2.24 g) and N,N-dimethylformamide (20 ml) was stirred at 190° C. for 16 hours. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and methyl (2-benzyloxy-3-chlorophenyl)acetate (740 mg, yield 45%) was obtained as a colorless oil from a fraction eluted with ethyl acetate-hexane (2:98, volume ratio).

¹H-NMR (CDCl₃)δ: 3.64 (2H, s), 3.65 (3H, s), 5.02 (2H, s), 7.05 (1H, t, J=8.0 Hz), 7.17 (1H, dd, J=8.0, 1.6 Hz), 7.34-7.42 (4H, m), 7.46-7.51 (2H, m).

### Reference Example 269

A mixture of methyl (2-benzyloxy-3-chlorophenyl)acetate (680 mg), 5% palladium-carbon (70 mg) and methanol (15 ml) was stirred overnight at room temperature under a hydrogen atmosphere. Palladium-carbon was removed by filtration and the filtrate was concentrated. The residue was subjected to silica gel column chromatography, and methyl (3-chloro-2-hydroxyphenyl)acetate (300 mg, yield 64%) was obtained as a colorless oil from a fraction eluted with ethyl acetate-hexane (1:9, volume ratio).

¹H-NMR (CDCl₃)δ: 3.70 (2H, s), 3.73 (3H, s), 6.28 (1H, s), 6.84 (1H, t, J=8.0 Hz), 7.08 (1H, dd, J=8.0, 0.8 Hz), 7.27 (1H, dd, J=8.0, 1.0 Hz).

### Reference Example 270

To a mixture of ethyl 3-[3-(1-methylethyl)-1H-pyrazol-4-yl]propanoate (1.50 g), 2-chloro-5-(trifluoromethyl)-1,3,4-thiadiazole (1.50 g) and N,N-dimethylformamide (15 ml) was added sodium hydride (60%, in oil, 0.34 g) at 0° C., and, after termination of hydrogen generation, the mixture was stirred at room temperature for 1 hour. The reaction mixture was poured into water, and extracted with ethyl acetate. The ethyl acetate layer was washed with saturated aqueous sodium chloride solution, dried (MgSO₄) and concentrated. The residue was subjected to silica gel column chromatography, and ethyl 3-{3-(1-methylethyl)-1-[5-(trifluoromethyl)-1,3,4-thiadiazol-2-yl]-1H-pyrazol-4-yl}propanoate (1.29 g, yield 50%) was obtained as a yellow oily substance from a fraction eluted with ethyl acetate-hexane (1:4, volume ratio).

¹H-NMR (CDCl₃)δ: 1.27 (3H, t, J=7.1 Hz), 1.30 (6H, d, J=7.0 Hz), 2.57-2.90 (4H, m), 3.01 (1H, septet, J=7.0 Hz), 4.17 (2H, q, J=7.1 Hz), 8.13 (1H, s).

### Reference Example 271

To a solution of ethyl 3-{3-(1-methylethyl)-1-[5-(trifluoromethyl)-1,3,4-thiadiazol-2-yl]-1H-pyrazol-4-yl}propanoate (1.29 g) in tetrahydrofuran (15 ml) was dropwise added a 1.5M solution (5.7 ml) of diisobutylaluminum hydride in toluene at 0° C., and the mixture was stirred at room temperature for 1 hour. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and 3-{3-(1-methylethyl)-1-[5-(trifluoromethyl)-1,3,4-thiadiazol-2-yl]-1H-pyrazol-4-yl}-1-propanol (0.82 g, yield 73%) was obtained as colorless crystals from a fraction eluted with ethyl acetate-hexane (2:3, volume ratio). The crystals were recrystallized from ethyl acetate-hexane. melting point: 89-90° C.

¹H-NMR (CDCl₃)δ: 1.30 (6H, d, J=7.0 Hz), 1.45 (1H, br s), 1.82-1.98 (2H, m), 2.62 (2H, t, J=7.8 Hz), 3.00 (1H, septet, J=7.0 Hz), 3.76 (2H, t, J=6.0 Hz), 8.13 (1H, s).

### Reference Example 272

To a mixture of 1-benzyl-4-[3-(1,3-dioxolan-2-yl)propyl]-1H-pyrazol-3-ol (21.8 g) and N,N-dimethylformamide (150 ml) potassium carbonate (16.7 g) was added diethylsulfuric acid (17.3 ml) at room temperature, and the mixture was stirred overnight. Saturated aqueous ammonium chloride solution was added to the reaction mixture, and the mixture was extracted with ethyl acetate. The ethyl acetate layer was washed with saturated aqueous sodium chloride solution, dried (MgSO₄) and concentrated. The obtained residue was subjected to silica gel column chromatography, and 1-benzyl-4-[3-(1,3-dioxolan-2-yl)propyl]-3-ethoxy-1H-pyrazole (19.5 g, yield 82%) was obtained as a yellow oily substance from a fraction eluted with ethyl acetate-hexane (1:3, volume ratio).

¹H NMR (CDCl₃) δ: 1.36 (3H, t, J=6.9 Hz), 1.57-1.74 (4H, m), 2.32-2.39 (2H, m), 3.80-3.98 (4H, m), 4.22 (2H, q, J=6.9 Hz), 4.82-4.87 (1H, m), 5.07 (2H, s), 6.93 (1H, s), 7.13-7.17 (2H, m), 7.23-7.35 (3H, m)

### Reference Example 273

A mixture of 3,3-dimethyl-2-butanone (6.19 ml) and bis(dimethylamino)methoxymethane (6.61 g) was heated under reflux for 10 hours. The reaction mixture was concentrated under reduced pressure. Hydrazine monohydrate (1.60 g) and n-butyl alcohol (24.9 ml) were added to the residue, and the mixture was heated under reflux for 7 hours. The reaction mixture was concentrated under reduced pressure to give 3-tert-butyl-1H-pyrazole (3.79 g, yield 61%) as a yellow oily substance.

¹H-NMR (CDCl₃) δ: 1.34 (9H, s), 6.10 (1H, d, J=2.0 Hz), 7.49 (1H, d, J=2.0 Hz), 10.3 (1H, br s).

### Reference Example 274

To a mixture of 3-tert-butyl-1H-pyrazole (3.72 g), 2-chloro-5-(trifluoromethyl)pyridine (5.45 g) and N-methylpyrrolidone (18.6 ml) was added sodium hydroxide (1.80 g) while stirring the mixture at room temperature. After allowing reaction as it was for 8 hours, water (38 ml) and 6N hydrochloric acid (80 ml) were added and the mixture was extracted with ethyl acetate. The extract was washed with water and concentrated under reduced pressure. The residue was subjected to silica gel column chromatography, and eluted with hexane and then with toluene to give 2-(3-tert-butyl-1H-pyrazol-1-yl)-5-(trifluoromethyl)pyridine (7.04 g, yield 87%) as a colorless oil.

¹H-NMR (CDCl₃) δ: 1.37 (9H, s), 6.37 (1H, d, J=2.6 Hz), 7.97 (1H, dd, J=8.7, 2.1 Hz), 8.08 (1H, d, J=8.7 Hz), 8.46 (1H, d, J=2.7 Hz), 8.6-8.7 (1H, m).

### Reference Example 275

Iodine (3.91 g) and successively diammonium cerium(IV) nitrate (844 mg) were added to a solution of 2-(3-tert-butyl-1H-pyrazol-1-yl)-5-(trifluoromethyl)pyridine (6.93 g) in acetonitrile (139 ml) while stirring the mixture at room temperature, and the reaction was continued for 5 hours. After the completion of the reaction, the reaction mixture was concentrated under reduced pressure. Water was added to the residue and the mixture was extracted with ethyl acetate. The organic layers were combined, washed with saturated aqueous sodium thiosulfate solution, dried (magnesium sulfate) and concentrated under reduced pressure to give 2-(3-tert-butyl-4-iodo-1H-pyrazol-1-yl)-5-(trifluoromethyl)pyridine (9.82 g, yield 96%) as a yellow oily substance.

¹H-NMR (CDCl₃) δ: 1.49 (9H, s), 7.97 (1H, dd, J=8.7, 2.1 Hz), 8.03(1H, d, J=8.7 Hz), 8.59 (1H, s), 8.6-8.7 (1H, m).

### Reference Example 276

A mixture of 2-(3-tert-butyl-4-iodo-1H-pyrazol-1-yl)-5-(trifluoromethyl)pyridine (8.68 g), palladium acetate (494 mg), triphenylphosphine (1.15 g), sodium acetate (3.61 g), benzyltriethylammonium chloride (5.01 g), methyl acrylate (7.89 ml) and N-methylpyrrolidone (86.8 ml) was stirred in a nitrogen stream at an outer temperature of 80° C. for 17 hours. The reaction mixture was cooled to room temperature and an insoluble material was removed by filtration. Water was added to the filtrate and the mixture was extracted with ethyl acetate. The organic layers were combined, washed with water, dried (sodium sulfate) and concentrated under reduced pressure. The residue was subjected to silica gel column chromatography, and methyl (E)-3-(3-tert-butyl-1-[5-(trifluoromethyl)-2-pyridinyl]-1H-pyrazol-4-yl)-2-propenoate (5.43 g, yield 70%) was obtained as a white solid and was obtained from a fraction eluted with hexane-ethyl acetate (19:1, volume ratio).

¹H-NMR (CDCl₃) δ: 1.44 (9H, s), 3.80 (3H, s), 6.26 (1H, d, J=15.8 Hz), 7.86 (1H, d, J=15.8 Hz), 8.00 (1H, dd, J=8.6, 2.2 Hz), 8.10 (1H, d, J=8.7 Hz), 8.65 (1H, d, J=2.2 Hz), 8.77 (1H, s).

### Reference Example 277

To a mixture of methyl (E)-3-{3-tert-butyl-1-[5-(trifluoromethyl)-2-pyridinyl]-1H-pyrazol-4-yl}-2-propenoate (3.00 g), 5% palladium-carbon (9.00 g), ethanol (50 ml) and tetrahydrofuran (10 ml) was added formic acid (25 ml), and the mixture was stirred for 2 hours with heating under reflux. The reaction mixture was cooled to room temperature and palladium-carbon was removed by filtration. The filtrate was concentrated under reduced pressure and the residue was diluted with ethyl acetate. The obtained ethyl acetate solution was washed with saturated aqueous sodium hydrogen carbonate and saturated brine, dried (MgSO₄) and concentrated to give a white solid. To a solution of the obtained solid in tetrahydrofuran (100 ml) was dropwise added a 0.93M solution (26.9 ml) of diisobutylaluminum hydride in hexane at 0° C. and the mixture was stirred at room temperature for 30 minutes. 1N Hydrochloric acid was added to the reaction mixture and the mixture was extracted with ethyl acetate. The extract was washed with saturated brine, dried (MgSO₄) and concentrated. The residue was subjected to silica gel column chromatography, and 3-{3-tert-butyl-1-[(5-(trifluoromethyl)-2-pyridinyl]-1H-pyrazol-4-yl}-1-propanol (2.74 g, yield 98%) was obtained as a white solid from a fraction eluted with ethyl acetate-hexane (1:4, volume ratio). The crystals were recrystallized from ethyl acetate-hexane to give colorless crystals. melting point: 69-70° C.

### Reference Example 278

A mixture of 3-tert-butyl-1H-pyrazole (2.00 g), sodium hydride (60% in oil, 773 mg) and N,N-dimethylformamide (80 ml) was stirred at room temperature for 30 minutes, and benzyl bromide (2.11 ml) was added. The mixture was stirred overnight. Water was added to the reaction mixture, and the mixture was extracted with ethyl acetate. The ethyl acetate layer was washed with saturated brine, dried (MgSO₄) and concentrated. The residue was subjected to silica gel column chromatography, and 1-benzyl-3-tert-butyl-1H-pyrazole (3.44 g, quantitative) was obtained as a colorless oil from a fraction eluted with ethyl acetate-hexane (1:5, volume ratio).

¹H-NMR (CDCl₃) δ: 1.33 (9H, s), 5.27 (2H, s), 6.10 (1H, d, J=2.4 Hz), 7.14-7.19 (3H, m), 7.24-7.37 (3H, m).

### Reference Example 279

A mixture of 1-benzyl-3-tert-butyl-1H-pyrazole (3.44 g), iodine (2.44 g), diammonium cerium(IV) nitrate (5.28 g) and acetonitrile (80 ml) was stirred overnight at room temperature. Water was added to the reaction mixture and the mixture was extracted with ethyl acetate. The ethyl acetate layer was washed with saturated aqueous sodium hydrosulfite solution and saturated brine, dried (MgSO₄) and concentrated to give 1-benzyl-3-tert-butyl-4-iodo-1H-pyrazole (5.34 g, yield 97%) as a green oily substance.

¹H-NMR (CDCl₃) δ: 1.44 (9H, s), 5.21 (2H, s), 7.18-7.26 (3H, m), 7.27-7.38 (3H, m).

### Reference Example 280

To a mixture of 1-benzyl-3-tert-butyl-4-iodo-1H-pyrazole (5.34 g), palladium(II) acetate (353 mg), triphenylphosphine (824 mg), benzyltriethylammonium chloride (3.58 g), methyl acrylate (5.63 ml) and 1-methyl-2-pyrrolidone (62.8 ml) was added sodium acetate (2.58 g) at room temperature, and the mixture was heated to 80° C. under an argon atmosphere. The mixture was stirred overnight at said temperature. The reaction mixture was cooled to room temperature, and an insoluble material was removed by filtration. Water was added to the filtrate, and the mixture was extracted with ethyl acetate. The extract was washed with water and saturated aqueous sodium hydrosulfite solution, dried (MgSO₄) and concentrated. The residue was subjected to silica gel column chromatography, and methyl (E)-3-(1-benzyl-3-tert-butyl-1H-pyrazol-4-yl)-2-propenoate (3.24 g, yield 69%) was obtained as a brown oily substance from a fraction eluted with ethyl acetate-hexane (1:9, volume ratio).

¹H-NMR (CDCl₃) δ: 1.40 (9H, s), 3.75 (3H, s), 5.23 (2H, s), 5.93 (1H, d, J=15.8 Hz), 7.20-7.28 (2H, m), 7.31-7.40 (3H, m), 7.47 (1H, s), 7.84 (1H, d, J=15.8 Hz).

### Reference Example 281

To a mixture of methyl (E)-3-(1-benzyl-3-tert-butyl-1H-pyrazol-4-yl)-2-propenoate (3.24 g), 5% palladium-carbon (9.00 g), ethanol (50 ml) and tetrahydrofuran (10 ml) was added formic acid (25 ml), and the mixture was stirred overnight while heating under reflux. The reaction mixture was cooled to room temperature and palladium-carbon was removed by filtration. The filtrate was concentrated and the residue was diluted with ethyl acetate. The obtained ethyl acetate solution was washed with saturated aqueous sodium hydrogen carbonate and saturated brine, dried (MgSO₄) and concentrated to give methyl 3-(3-tert-butyl-1H-pyrazol-4-yl)propanoate (2.08 g, yield 91%) as a colorless oil.

¹H-NMR (CDCl₃) δ: 1.38 (9H, s), 2.57-2.65 (2H, m), 2.88-2.95 (2H, m), 3.69 (3H, s), 7.33 (1H, s).

### Reference Example 282

To a mixture of 3-hydroxy-2-methylisonicotinic acid (4.52 g), potassium carbonate (18.6 g) and N,N-dimethylformamide (200 ml) was added benzyl bromide (15.9 ml) at room temperature and the mixture was stirred for 3.5 days. Saturated aqueous sodium hydrogen carbonate was added to the reaction mixture and the mixture was extracted with ethyl acetate. The ethyl acetate layer was washed with water and saturated brine, dried (MgSO₄) and concentrated. The residue was subjected to silica gel column chromatography, and benzyl 3-(benzyloxy)-2-methylisonicotinate (4.18 g, yield 43%) was obtained as a colorless oil from a fraction eluted with ethyl acetate-hexane (3:7, volume ratio).

¹H-NMR (CDCl₃) δ: 2.55 (3H, s), 4.94 (2H, s), 5.34 (2H, s), 7.30-7.44 (10H, m), 7.48 (1H, d, J=5.1 Hz), 8.35 (1H, d, J=5.1 Hz).

### Reference Example 283

To a solution of benzyl 3-(benzyloxy)-2-methylisonicotinate (4.18 g) in tetrahydrofuran (100 ml) was dropwise added a 0.93M solution (45.0 ml) of diisobutylaluminum hydride in hexane at 0° C. and the mixture was stirred at said temperature for 1 hour. Sodium sulfate 10 hydrate (13.5 g) was added to the reaction mixture and the mixture was stirred overnight at room temperature. The resulting insoluble material was removed by filtration and the filtrate was concentrated. The residue was subjected to silica gel column chromatography, and [3-(benzyloxy)-2-methyl-4-pyridinyl]methanol (2.50 g, yield 87%) was obtained as a white solid from a fraction eluted with ethyl acetate-hexane (4:1, volume ratio). The crystals were recrystallized from ethyl acetate-hexane to give colorless crystals. melting point: 130-131° C.

### Reference Example 284

To a mixture of [3-(benzyloxy)-2-methyl-4-pyridinyl]methanol (2.40 g), acetone cyanohydrin (2.14 ml), tributylphosphine (5.23 ml) and tetrahydrofuran (200 ml) was added 1,1′-azodicarbonyldipiperidine (5.30 g) at room temperature and the mixture was stirred for 1 hour. The reaction solution was concentrated. The residue was subjected to silica gel column chromatography, and a orange oily substance was obtained from a fraction eluted with ethyl acetate-hexane (1:1, volume ratio). A mixture of the obtained oily substance, potassium hydroxide (2.95 g), water (25 ml) and ethanol (100 ml) was stirred overnight while heating under reflux. The reaction mixture was concentrated, and the residue was diluted with water. The obtained aqueous solution was washed with ether, carefully neutralized with conc. hydrochloric acid and extracted with ethyl acetate. The extract was washed with saturated brine, dried (MgSO₄) and concentrated to give [3-(benzyloxy)-2-methyl-4-pyridinyl]acetic acid (1.41 g, yield 52%) as a brown solid.

¹H-NMR (CDCl₃) δ: 2.54 (3H, s), 3.69 (2H, s), 4.90 (2H, s), 7.20 (1H, d, J=5.1 Hz), 7.30-7.48 (5H, m), 8.25 (1H, d, J=5.1 Hz).

### Reference Example 285

To a mixture of [3-(benzyloxy)-2-methyl-4-pyridinyl]acetic acid (1.41 g), potassium carbonate (2.28 g) and N,N-dimethylformamide (50 ml) was added methyl iodide (1.02 ml) at room temperature and the mixture was stirred for 2 hours. Saturated aqueous sodium hydrogen carbonate was added to the reaction mixture and the mixture was extracted with ethyl acetate. The extract was washed with water and saturated brine, dried (MgSO₄) and concentrated. The residue was subjected to silica gel column chromatography, and methyl [3-(benzyloxy)-2-methyl-4-pyridinyl]acetate (1.46 g, yield 98%) was obtained as a yellow oily substance from a fraction eluted with ethyl acetate-hexane (3:7, volume ratio).

¹H-NMR (CDCl₃) δ: 2.57 (3H, s), 3.63 (2H, s), 3.67 (3H, s), 4.87 (2H, s), 7.07 (1H, d, J=5.2 Hz), 7.30-7.50 (5H, m), 8.25 (1H, d, J=5.2 Hz).

### Reference Example 286

A mixture of methyl [3-(benzyloxy)-2-methyl-4-pyridinyl]acetate (1.46 g), 5% palladium-carbon (500 mg) and ethanol (60 ml) was stirred overnight at room temperature under a hydrogen atmosphere. Palladium-carbon was removed by filtration and the filtrate was concentrated. The residue was subjected to silica gel column chromatography, and methyl (3-hydroxy-2-methyl-4-pyridinyl)acetate (671 mg, yield 69%) was obtained as a yellow oily substance from a fraction eluted with methanol-ethyl acetate (1:9, volume ratio).

¹H-NMR (CDCl₃) δ: 2.51 (3H, s), 3.70 (2H, s), 3.78 (3H, s), 6.89 (1H, d, J=5.0 Hz), 8.01 (1H, d, J=5.0 Hz).

### Reference Example 287

To a mixture of {3-isopropyl-1-[5-(trifluoromethyl)-2-pyridyl]-1H-pyrazol-4-yl}methanol (3.34 g), acetone cyanohydrin (2.20 g), tributylphosphine (4.76 g) and tetrahydrofuran (50 ml) was added 1,1′-azodicarbonyldipiperidine (5.90 g) at room temperature and the mixture was stirred for 2 hours. The reaction solution was concentrated. The residue was subjected to silica gel column chromatography, and {3-isopropyl-1-[5-(trifluoromethyl)-2-pyridyl]-1H-pyrazol-4-yl}acetonitrile (3.30 g, yield 96%) was obtained as a yellow oily substance from a fraction eluted with ethyl acetate-hexane (2:3, volume ratio).

¹H-NMR (CDCl₃) δ: 1.36 (6H, d, J=7.0 Hz), 3.04 (1H, septet, J=6.9 Hz), 3.61 (2H, s), 7.95-8.10 (2H, m), 8.56 (1H, s), 8.62-8.65 (1H, m).

### Reference Example 288

A mixture of {3-isopropyl-1-[5-(trifluoromethyl)-2-pyridyl]-1H-pyrazol-4-yl}acetonitrile (3.30 g), 6N aqueous sodium hydroxide solution (11 ml), ethanol (20 ml) and tetrahydrofuran (20 ml) was ref luxed overnight. The reaction mixture was concentrated and water (80 ml) was added. The mixture was washed with diethyl ether. The aqueous layer was acidified by adding conc. hydrochloric acid and the mixture was extracted with ethyl acetate. The ethyl acetate layer was washed with saturated brine, dried (MgSO₄) and concentrated. A mixture of the obtained oily substance, conc. sulfuric acid (0.1 ml) and ethanol (40 ml) was refluxed overnight. The reaction mixture was concentrated and aqueous sodium hydrogen carbonate was added to the residue. The mixture was extracted with ethyl acetate. The ethyl acetate layer was washed with saturated brine, dried (MgSO₄) and concentrated. The residue was subjected to silica gel column chromatography, and ethyl {3-isopropyl-1-[5-(trifluoromethyl)-2-pyridyl]-1H-pyrazol-4-yl}acetate (2.78 g, yield 73%) was obtained as a yellow oily substance from a fraction eluted with ethyl acetate-hexane (1:4, volume ratio).

¹H-NMR (CDCl₃) δ: 1.28 (3H, t, J=7.1 Hz), 1.32 (6H, d, J=6.9 Hz), 3.02 (1H, septet, J=6.9 Hz), 3.53 (2H, s), 4.18 (2H, q, J=7.1 Hz), 7.91-7.97 (1H, m), 8.04 (1H, d, J=8.4 Hz), 8.46 (1H, s), 8.60-8.62 (1H, m).

### Reference Example 289

To a mixture of ethyl {3-isopropyl-1-[5-(trifluoromethyl)-2-pyridyl]-1H-pyrazol-4-yl}acetate (2.68 g) and tetrahydrofuran (35 ml) was slowly added a 1.5M solution (13.0 ml) of diisobutylaluminum hydride in toluene at 0° C. and the mixture was stirred at room temperature for 1 hour. The reaction mixture was poured into dilute hydrochloric acid and the mixture was extracted with ethyl acetate. The ethyl acetate layer was washed with saturated brine, dried (MgSO₄) and concentrated to give 2-{3-isopropyl-1-[5-(trifluoromethyl)-2-pyridyl]-1H-pyrazol-4-yl}-1-ethanol (1.21 g, yield 51%) as colorless crystals. The crystals were recrystallized from ethyl acetate-hexane. melting point: 74-75° C.

¹H-NMR (CDCl₃) δ: 1.34 (6H, d, J=7.0 Hz), 1.58 (1H, t, J=5.8 Hz), 2.78 (2H, td, J=6.6, 0.8 Hz), 3.05 (1H, septet, J=6.9 Hz), 3.87 (2H, q, J=6.4 Hz), 7.95 (1H, dd, J-9.0, 2.0 Hz), 8.04 (1H, d, J=8.8 Hz), 8.36 (1H, s), 8.59-8.61 (1H, m).

### Reference Example 290

To a mixture of ethyl 3-[3-(1-ethylpropyl)-1H-pyrazol-4-yl]propanoate (3.34 g), 2,5-dibromopyridine (3.65 g) and N,N-dimethylformamide (20 ml) was added 60% sodium hydride (0.67 g) and the mixture was stirred at 100° C. for 4 hours. The reaction mixture was poured into dilute hydrochloric acid and extracted with ethyl acetate. The ethyl acetate layer was washed with saturated brine, dried (MgSO₄) and concentrated. Ethanol (20 ml) and conc. sulfuric acid (0.1 ml) were added to the residue and the mixture was stirred at 50° C. for 6 hours. The reaction mixture was poured into aqueous sodium hydrogen carbonate solution and the mixture was extracted with ethyl acetate. The ethyl acetate layer was washed with saturated brine, dried (MgSO₄) and concentrated. The residue was subjected to silica gel column chromatography, and ethyl 3-[1-(5-bromo-2-pyridinyl)-3-(1-ethylpropyl)-1H-pyrazol-4-yl]propanoate (3.90 g, yield 71%) was obtained as a white powder from a fraction eluted with ethyl acetate-hexane (1:9, volume ratio).

¹H-NMR (CDCl₃) δ: 0.86 (6H, t, J=7.6 Hz), 1.26 (3H, t, J=7.2 Hz), 1.64-1.80 (4H, m), 2.56-2.64 (3H, m), 2.78-2.81 (2H, m), 4.16 (2H, q, J=7.2 Hz), 7.82-7.83 (2H, m), 8.20 (1H, s), 8.38-8.39 (1H, m).

### Reference Example 291

To a solution of ethyl 3-[1-(5-bromo-2-pyridinyl)-3-(1-ethylpropyl)-1H-pyrazol-4-yl]propanoate (3.80 g) in tetrahydrofuran (50 ml) was dropwise added a 1.0 M solution (30 ml) of diisobutylaluminum hydride in hexane at 0° C. and the mixture was stirred at room temperature for 2 hours. The reaction mixture was poured into dilute hydrochloric acid, and the mixture was extracted with ethyl acetate. The ethyl acetate layer was washed with saturated brine, dried (MgSO₄) and concentrated. The residue was subjected to silica gel column chromatography, and 3-[1-(5-bromo-2-pyridinyl)-3-(1-ethylpropyl)-1H-pyrazol-4-yl]-1-propanol (2.60 g, yield 77%) was obtained as a white powder from a fraction eluted with ethyl acetate-hexane (3:7, volume ratio).

¹H-NMR (CDCl₃) δ: 0.86 (6H, t, J=7.6 Hz), 1.30 (1H, t, J=5.2 Hz), 1.66-1.80 (4H, m), 1.87-1.91 (2H, m), 2.54-2.60 (3H, m), 3.72-3.76 (2H, m), 7.83 (2H, m), 8.20 (1H, s), 8.38-8.39 (1H, m).

### Reference Example 292

To a mixture of 2-isopropylphenol (13.62 g), tributylamine (7.41 g) and toluene (50 ml) was added tin tetrachloride (1.18 ml) at room temperature and the mixture was stirred for 30 minutes. Paraformaldehyde (6.60 g) was added, and the mixture was stirred overnight at 100° C. The reaction mixture was poured into dilute hydrochloric acid and extracted with ethyl acetate. The ethyl acetate layer was washed with saturated brine, dried (MgSO₄) and concentrated. The residue was subjected to silica gel column chromatography, and 2-hydroxy-3-isopropylbenzaldehyde (9.90 g, yield 60%) was obtained as a colorless oil from a fraction eluted with hexane

¹H-NMR (CDCl₃) δ: 1.25 (6H, t, J=6.8 Hz), 3.30-3.40 (1H, m), 6.99 (1H, t, J=7.6 Hz), 7.40 (1H, dd, J=7.6, 1.6 Hz), 7.47 (1H, dd, J=7.6, 1.6 Hz), 9.89 (1H, s), 11.37 (1H, s).

### Reference Example 293

A mixture of 2-hydroxy-3-isopropylbenzaldehyde (8.10 g), benzyl bromide (10.12 g), potassium carbonate (8.18 g) and N,N-dimethylformamide (30 ml) was stirred at 50° C. for 1 hour. The reaction mixture was poured into dilute hydrochloric acid, and extracted with ethyl acetate. The ethyl acetate layer was washed with saturated brine, dried (MgSO₄) and concentrated. The residue was subjected to silica gel column chromatography, and 2-benzyloxy-3-isopropylbenzaldehyde (11.70 g, yield 93%) was obtained as a colorless oil from a fraction eluted with ethyl acetate-hexane (2:98, volume ratio).

¹H-NMR (CDCl₃) δ: 1.25 (6H, d, J=6.8 Hz), 3.40-3.46 (1H, m), 4.97(2H, s), 7.25 (1H, t, J=7.8 Hz), 7.36-7.44 (5H, m), 7.57 (1H, dd, J=7.8, 1.8 Hz), 7.71 (1H, dd, J=7.8, 1.8 Hz), 10.30 (1H, s).

### Reference Example 294

To a mixture of 2-benzyloxy-3-isopropylbenzaldehyde (11.50 g), methyl (methylthio)methyl sulfoxide (11.23 g) and tetrahydrofuran (100 ml) was added a 40% solution (2.00 ml) of benzyltrimethylammonium hydroxide in methanol at room temperature and the mixture was stirred at 65° C. for 2 hours. The reaction solution was concentrated. The residue was subjected to silica gel column chromatography, and 2-[2-(benzyloxy)-3-isopropylphenyl]-1-(methylthio)vinyl methyl sulfoxide (13.50 g, yield 83%) was obtained as a yellow oily substance from a fraction eluted with ethyl acetate-hexane (1:1, volume ratio).

¹H-NMR (CDCl₃) δ: 1.22 (6H, dd, J=6.8, 0.8 Hz), 2.30 (3H, s), 2.72 (3H, s), 3.35-3.43 (1H, m), 4.76-4.82 (2H, m), 7.19 (1H, t, J=7.8 Hz), 7.32-7.43 (4H, m), 7.49-7.52 (2H, m), 7.93 (1H, dd, J=7.8, 1.6 Hz), 8.05 (1H, s).

### Reference Example 295

A mixture of 2-[2-(benzyloxy)-3-isopropylphenyl]-1-(methylthio)vinyl methyl sulfoxide (13.30 g) and a 10% solution (100 ml) of hydrogen chloride in methanol was refluxed for 2 hours. The reaction solution was concentrated and ethyl acetate and aqueous sodium hydrogen carbonate were added to the residue and the mixture was extracted. The ethyl acetate layer was washed with saturated brine, dried (MgSO₄) and concentrated. The residue was subjected to silica gel column chromatography, and methyl (2-benzyloxy-3-isopropylphenyl)acetate (8.90 g, yield 80%) was obtained as a colorless oil from a fraction eluted with ethyl acetate-hexane (4:96, volume ratio).

¹H-NMR (CDCl₃) δ: 1.24 (6H, d, J=6.8 Hz), 3.32-3.44 (1H, m), 3.67 (3H, s), 3.71 (2H, s), 4.84 (2H, s), 7.11-7.14 (2H, m), 7.24 (1H, dd, J=6.4, 3.2 Hz), 7.35-7.43 (3H, m), 7.47-7.49 (2H, m).

### Reference Example 296

A mixture of methyl (2-benzyloxy-3-isopropylphenyl)acetate (8.40 g), 5% palladium-carbon (0.80 g) and methanol (80 ml) was stirred overnight at room temperature under a hydrogen atmosphere. Palladium-carbon was removed by filtration, and the filtrate was concentrated. The residue was subjected to silica gel column chromatography, and methyl (2-hydroxy-3-isopropylphenyl)acetate (4.80 g, yield 82%) was obtained as a colorless oil from a fraction eluted with ethyl acetate-hexane (1:9, volume ratio).

¹H-NMR (CDCl₃) δ: 1.24 (6H, d, J=6.8 Hz), 3.32-3.43 (1H, m), 3.68 (2H, s), 3.75 (3H, s), 6.83 (1H, t, J=7.6 Hz), 6.93 (1H, dd, J=7.6, 1.2 Hz), 7.16 (1H, dd, J=7.6, 2.0 Hz), 7.66 (1H, s).

### Reference Example 297

To a mixture of ethyl 3-(3-isopropyl-1H-pyrazol-4-yl)propanoate (0.50 g), 2-chloro-3-(trifluoromethyl)pyridine (0.43 g) and N,N-dimethylformamide (10 ml) was added 60% sodium hydride (0.1 g) at 100° C. and the mixture was stirred for 1 hour. The reaction mixture was poured into dilute ydrochloric acid, and extracted with ethyl acetate. The ethyl acetate layer was washed with saturated brine, dried (MgSO4) and concentrated. Ethanol (10 ml) and conc. sulfuric acid (0.05 ml) were added to the residue and the mixture was stirred at 70° C. for 2 hours. The reaction mixture was poured into an aqueous sodium hydrogen carbonate solution, and extracted with ethyl acetate. The ethyl acetate layer was washed with saturated brine, dried (MgSO₄) and concentrated. The residue was subjected to silica gel column chromatography, and ethyl 3-{3-isopropyl-1-[3-(trifluoromethyl)-2-pyridinyl]-1H-pyrazol-4-yl}propanonate (0.60 g, yield 71%) was obtained as a colorless oil from a fraction eluted with ethyl acetate-hexane (1:9, volume ratio).

¹H-NMR (CDCl₃) δ: 1.26 (3H, t, J=7.2 Hz), 1.32 (6H, d, J=7.2 Hz), 2.62-2.66 (2H, m), 2.82-2.86 (2H, m), 2.99-3.06 (1H, m), 4.15 (2H, q, J=7.2 Hz), 7.32 (1H, dd, J=8.0, 4.8 Hz), 7.96 (1H, s), 8.14 (1H, dd, J=8.0, 1.6 Hz), 8.59 (1H, dd, J=4.8, 1.6 Hz).

### Reference Example 298

To a solution of ethyl 3-{3-isopropyl-1-[3-(trifluoromethyl)-2-pyridinyl]-1H-pyrazol-4-yl}propanoate (0.60 g) in tetrahydrofuran (10 ml) was dropwise added a 1.0 M solution (10 ml) of diisobutylaluminum hydride in hexane at 0° C. and the mixture was stirred at room temperature for 2 hours. The reaction mixture was poured into dilute hydrochloric acid, and extracted with ethyl acetate. The ethyl acetate layer was washed with saturated brine, dried (MgSO₄) and concentrated. The residue was subjected to silica gel column chromatography, and 3-{(3-isopropyl-1-[3-(trifluoromethyl)-2-pyridinyl]-1H-pyrazol-4-yl}-1-propanol (0.44 g, yield 83%) was obtained as a colorless oil from a fraction eluted with ethyl acetate-hexane (3:7, volume ratio).

¹H-NMR (CDCl₃) δ: 1.32 (6H, d, J=6.8 Hz), 1.88-1.95 (2H, m) 2.58-2.62 (2H, m), 2.98-3.05 (1H, m), 3.73-3.76 (2H, m), 7.29-7.33 (1H, m), 7.96 (1H, s), 8.14 (1H, dd, J=8.2, 1.2 Hz), 8.59 (1H, dd, J=4.8, 1.6 Hz).

### Reference Example 299

To a mixture of ethyl 3-(3-isopropyl-1H-pyrazol-4-yl)propanoate (0.50 g), 2-chloro-4-(trifluoromethyl)pyridine (0.43 g) and N,N-dimethylformamide (10 ml) was added 60% sodium hydride (0.19 g) at 100° C. and the mixture was stirred for 1 hour. The reaction mixture was poured into dilute hydrochloric acid, and extracted with ethyl acetate. The ethyl acetate layer was washed with saturated brine, dried (MgSO₄) and concentrated. Ethanol (10 ml) and conc. sulfuric acid (0.05 ml) were added to the residue and the mixture was stirred at 70° C. for 2 hours. The reaction mixture was poured into an aqueous sodium hydrogen carbonate solution, and extracted with ethyl acetate. The ethyl acetate layer was washed with saturated brine, dried (MgSO₄) and concentrated. The residue was subjected to silica gel column chromatography, and ethyl 3-{3-isopropyl-1-[4-(trifluoromethyl)-2-pyridinyl]-1H-pyrazol-4-yl}propanoate (0.54 g, yield 64%) was obtained as a colorless oil from a fraction eluted with ethyl acetate-hexane (1:9, volume ratio).

¹H-NMR (CDCl₃) δ: 1.26 (3H, t, J=7.2 Hz), 1.34 (6H, d, J=7.2 Hz), 2.62-2.66 (2H, m), 2.81-2.85 (2H, m), 3.01-3.08 (1H, m), 4.16(2H, q, J=7.2 Hz), 7.28 (1H, dd, J=5.2, 1.2 Hz), 8.14 (1H, s), 8.27 (1H, s), 8.50 (1H, d, J-5.2 Hz).

### Reference Example 300

To a solution of ethyl 3-{3-isopropyl-1-[4-(trifluoromethyl)-2-pyridinyl]-1H-pyrazol-4-yl}propanoate (0.45 g) in tetrahydrofuran (6 ml) was dropwise added a 1.0 M solution (5 ml) of diisobutylaluminum hydride in hexane at 0° C. and the mixture was stirred at room temperature for 2 hours. The reaction mixture was poured into dilute hydrochloric acid, and extracted with ethyl acetate. The ethyl acetate layer was washed with saturated brine, dried (MgSO₄) and concentrated. The residue was subjected to silica gel column chromatography, and 3-{3-isopropyl-1-[4-(trifluoromethyl)-2-pyridinyl]-1H-pyrazol-4-yl}-1-propanol (0.37 g, yield 93%) was obtained as a colorless oil from a fraction eluted with ethyl acetate-hexane (3:7, volume ratio).

¹H-NMR (CDCl₃) δ: 1.34 (6H, d, J=6.8 Hz), 1.89-1.95 (2H, m), 2.58-2.62 (2H, m), 3.01-3.08 (1H, m), 3.75 (2H, m), 7.28 (1H, d, J=5.2 Hz), 8.15 (1H, s), 8.26 (1H, s), 8.50 (1H, d, J=5.2 Hz).

### Reference Example 301

To a mixture of ethyl 3-(3-isopropyl-1H-pyrazol-4-yl)propanoate (0.63 g), 2-chloro-6-(trifluoromethyl)pyridine (0.55 g) and N,N-dimethylformamide (10 ml) was added 60% sodium hydride (0.20 g) at 100° C. and the mixture was stirred for 1 hour. The reaction mixture was poured into dilute hydrochloric acid, and extracted with ethyl acetate. The ethyl acetate layer was washed with saturated brine, dried (MgSO₄) and concentrated. Ethanol (10 ml) and conc. sulfuric acid (0.05 ml) were added to the residue and the mixture was stirred at 70° C. for 2 hours. The reaction mixture was poured into an aqueous sodium hydrogen carbonate solution, and extracted with ethyl acetate. The ethyl acetate layer was washed with saturated brine, dried (MgSO₄) and concentrated. The residue was subjected to silica gel column chromatography, and ethyl 3-{3-isopropyl-1-[6-(trifluoromethyl)-2-pyridinyl]-1H-pyrazol-4-yl}propanoate (0.67 g, yield 63%) was obtained as a colorless oil from a fraction eluted with ethyl acetate-hexane (1:9, volume ratio).

¹H-NMR (CDCl₃) δ: 1.28 (3H, t, J=7.2 Hz), 1.33 (6H, d, J=7.2 Hz), 2.63-2.67 (2H, m), 2.83 (2H, t, J=8.0 Hz), 3.01-3.07 (1H, m), 4.17 (2H, q, J=7.2 Hz), 7.44 (1H, d, J=7.6 Hz), 7.88-7.92 (1H, m), 8.11 (1H, d, J=8.4 Hz), 8.30 (1H, s).

### Reference Example 302

To a solution of ethyl 3-{3-isopropyl-1-[6-(trifluoromethyl)-2-pyridinyl]-1H-pyrazol-4-yl}propanoate (0.47 g) in tetrahydrofuran (5 ml) was dropwise added a 1.0 M solution (4 ml) of diisobutylaluminum hydride in hexane at 0° C. and the mixture was stirred at room temperature for 2 hours. The reaction mixture was poured into dilute hydrochloric acid, and extracted with ethyl acetate. The ethyl acetate layer was washed with saturated brine, dried (MgSO₄) and concentrated. The residue was subjected to silica gel column chromatography, and 3-{3-isopropyl-1-[6-(trifluoromethyl)-2-pyridinyl]-1H-pyrazol-4-yl}-1-propanol (0.38 g, yield 92%) was obtained as a white powder from a fraction eluted with ethyl acetate-hexane (3:7, volume ratio).

¹H-NMR (CDCl₃) δ: 1.33 (6H, d, J=7.2 Hz), 1.89-1.96 (2H, m) 2.57-2.61 (2H, m), 3.00-3.07 (1H, m), 3.73-3.78 (2H, m), 7.44 (1H, d, J=7.6 Hz), 7.88-7.91 (1H, m), 8.12 (1H, d, J=8.4 Hz), 8.30 (1H, s).

### Reference Example 303

15 To a mixture of 2-benzyloxy-3-methylbenzaldehyde (37.00 g), methyl (methylthio)methyl sulfoxide (40.60 g) and tetrahydrofuran (400 ml) was added a 40% solution (8.00 ml) of benzyltrimethylammonium hydroxide in methanol at room temperature and the mixture was stirred at 65° C. for 2 hours. The reaction solution was concentrated. The residue was subjected to silica gel column chromatography, and 2-[2-(benzyloxy)-3-methylphenyl]-1-(methylthio)vinyl methyl sulfoxide (47.00 g, yield 86%) was obtained as a yellow oily substance from a fraction eluted with ethyl acetate-hexane (1:1, volume ratio).

¹H-NMR (CDCl₃) δ: 2.28 (3H, s), 2.32 (3H, s), 2.72 (3H, s), 4.81 (2H, s), 7.11 (1H, t, J-7.6 Hz), 7.23-7.26 (1H, m), 7.33-7.42 (3H, m), 7.48-7.51 (2H, m), 7.93-7.96 (1H, m), 8.02 (1H, s).

### Reference Example 304

To a mixture of 2-benzyloxy-3-methoxybenzaldehyde (55.00 g), methyl (methylthio)methyl sulfoxide (57.10 g) and tetrahydrofuran (400 ml) was added a 40% solution (10.00 ml) of benzyltrimethylammonium hydroxide in methanol at room temperature and the mixture was stirred at 65° C. for 2 hours. The reaction solution was concentrated. The residue was subjected to silica gel column chromatography, and 2-[2-(benzyloxy)-3-methoxyphenyl]-1-(methylthio)vinyl methyl sulfoxide (72.80 g, yield 91%) was obtained as a yellow oily substance from a fraction eluted with ethyl acetate-hexane (1:1, volume ratio).

¹H-NMR (CDCl₃) δ: 2.17 (3H, s), 2.68 (3H, s), 3.91 (3H, s), 5.03-5.04 (2H, m), 6.97 (1H, dd, J=8.0, 1.6 Hz), 7.10 (1H, t, J=8.0 Hz), 7.29-7.36 (3H, m), 7.44-7.46 (2H, m), 7.68(1H, dd, J=8.0, 1.2 Hz), 7.92(1H, s).

### Reference Example 305

To a mixture of methyl 3-(3-tert-butyl-1H-pyrazol-4-yl)propanoate (0.75 g), 3-chloro-6-(trifluoromethyl)pyridazine (0.98 g) and N,N-dimethylformamide (10 ml) was added 60% sodium hydride (0.17 g), and the mixture was stirred at room temperature for 1 hour. The reaction mixture was poured into dilute hydrochloric acid, and extracted with ethyl acetate. The ethyl acetate layer was washed with saturated brine, dried (MgSO₄) and concentrated. The residue was subjected to silica gel column chromatography, and methyl 3-{3-tert-butyl-1-[6-(trifluoromethyl)pyridazin-3-yl]-1H-pyrazol-4-yl}propanoate (1.08 g, yield 85%) was obtained as a colorless oil from a fraction eluted with ethyl acetate-hexane (1:10, volume ratio).

¹H-NMR (CDCl₃) δ: 1.41 (9H, s), 2.72 (2H, t, J=7.5 Hz), 3.01 (2H, t, J=7.5 Hz), 3.73 (3H, s), 7.83 (1H, d, J=9.6 Hz), 8.28 (1H, d, J=9.6 Hz), 8.50 (1H, s).

### Reference Example 306

To a solution of methyl 3-{3-tert-butyl-1-[6-(trifluoromethyl)pyridazin-3-yl]-1H-pyrazol-4-yl}propanoate (1.08 g) in tetrahydrofuran (50 ml) was dropwise added a 0.93 M solution (8.1 ml) of diisobutylaluminum hydride in hexane at 0° C. and the mixture was stirred at room temperature for 1 hour. The reaction mixture was poured into dilute hydrochloric acid, and extracted with ethyl acetate. The ethyl acetate layer was washed with saturated brine, dried (MgSO₄) and concentrated. The residue was subjected to silica gel column chromatography, and 3-{3-tert-butyl-1-[6-(trifluoromethyl)pyridazin-3-yl]-1H-pyrazol-4-yl}-1-propanol (0.66 g, yield 66%) was obtained as a colorless oil from a fraction eluted with ethyl acetate-hexane (1:2, volume ratio).

¹H-NMR (CDCl₃) δ: 1.41 (9H, s), 1.92-2.06 (2H, m), 2.77 (2H, t, J=7.8 Hz), 3.80 (2H, t, J=6.0 Hz), 7.83 (1H, d, J=9.3 Hz), 8.29 (1H, d, J=9.3 Hz), 8.52 (1H, s).

### Reference Example 307

To a solution of methyl 3-(3-tert-butyl-1H-pyrazol-4-yl)propanoate (580 mg) in N,N-dimethylformamide (15 ml) was added 60% sodium hydride (132 mg), and the mixture was stirred at room temperature for 30 minutes. 2,5-Dibromopyridine (784 mg) was added to the reaction mixture and the mixture was stirred at 100° C. for 1 hour. The reaction mixture was poured into water, neutralized with 2N hydrochloric acid and extracted with ethyl acetate. The ethyl acetate layer was washed with saturated brine, dried (MgSO₄) and concentrated. Ethanol (10 ml) and conc. sulfuric acid (0.05 ml) were added to the residue and the mixture was stirred at 70° C. for 2 hours. The reaction mixture was poured into an aqueous sodium hydrogen carbonate solution, and extracted with ethyl acetate. The ethyl acetate layer was washed with saturated brine, dried (MgSO₄) and concentrated. The residue was subjected to silica gel column chromatography, and crystals were obtained from a fraction eluted with ethyl acetate-hexane (1:9, volume ratio). The crystals were recrystallized from hexane to give ethyl 3-[1-(5-bromo-2-pyridinyl)-3-tert-butyl-1H-pyrazol-4-yl]propanoate (560 mg, yield 55%). melting point: 94-95° C.

### Reference Example 308

To a solution of ethyl 3-[1-(5-bromo-2-pyridinyl)-3-tert-butyl-1H-pyrazol-4-yl]propanoate (550 mg) in tetrahydrofuran (20 ml) was dropwise added a 1.0 M solution (5 ml) of diisobutylaluminum hydride in hexane at 0° C. and the mixture was stirred at room temperature for 40 minutes. The reaction mixture was poured into dilute hydrochloric acid, and extracted with ethyl acetate. The ethyl acetate layer was washed with saturated brine, dried (MgSO₄) and concentrated. The residue was subjected to silica gel column chromatography, and 3-[1-(5-bromo-2-pyridinyl)-3-tert-butyl-1H-pyrazol-4-yl]-1-propanol (455 mg, yield 90%) was obtained as a colorless oil from a fraction eluted with ethyl acetate-hexane (2:3, volume ratio).

¹H-NMR (CDCl₃) δ: 1.32 (1H, t, J=5.2 Hz), 1.40 (9H, s), 1.9-2.05 (2H, m), 2.65-2.8 (2H, m), 3.7-3.85 (2H, m), 7.83 (1H, br s), 7.84 (1H, s), 8.2-8.22 (1H, m), 8.35-8.4 (1H, m).

### Reference Example 309

To a solution of methyl 3-(3-tert-butyl-1H-pyrazol-4-yl)propanoate (0.75 g) in N,N-dimethylformamide (10 ml) was added 60% sodium hydride (0.17 g) and the mixture was stirred at room temperature for 30 minutes. 2,5-Dichloropyridine (0.80 g) was added to the reaction mixture and the mixture was stirred at 90° C. for 4 hours. 0.1N Hydrochloric acid was poured into the reaction mixture, and the mixture was extracted with ethyl acetate. The ethyl acetate layer was washed with saturated brine, dried (MgSO₄) and concentrated. The residue was subjected to silica gel column chromatography, and methyl 3-[3-tert-butyl-1-(5-chloropyridin-2-yl)-1H-pyrazol-4-yl]propanoate (0.95 g, yield 81%) was obtained as a colorless oil from a fraction eluted with ethyl acetate-hexane (5:95, volume ratio).

¹H-NMR (CDCl₃) δ: 1.40 (9H, s), 2.64-2.73 (2H, m), 2.94 (2H, m), 3.71 (3H, s), 7.69 (1H, dd, J=8.8, 2.6 Hz), 7.88 (1H, d, J=8.8 Hz), 8.20 (1H, s), 8.28 (1H, d, J=2.6 Hz).

### Reference Example 310

To a solution of methyl 3-[3-tert-butyl-1-(5-chloropyridin-2-yl)-1H-pyrazol-4-yl]propanoate (0.95 g) in tetrahydrofuran (50 ml) was dropwise added a 0.93 M solution (8.0 ml) of diisobutylaluminum hydride in hexane at 0° C. and the mixture was stirred at 0° C. for 1 hour. 1N Hydrochloric acid was poured into the reaction mixture and the mixture was extracted with ethyl acetate. The ethyl acetate layer was washed with saturated brine, dried (MgSO₄) and concentrated. The residue was subjected to silica gel column chromatography, and 3-[3-tert-butyl-1-(5-chloropyridin-2-yl)-1H-pyrazol-4-yl]-1-propanol (0.48 g, yield 55%) was obtained as a colorless oil from a fraction eluted with ethyl acetate-hexane (1:3, volume ratio).

¹H-NMR (CDCl₃) δ: 1.34 (1H, t, J=5.2 Hz), 1.40 (9H, s), 1.87-2.02 (2H, m), 2.68-2.76 (2H, m), 3.72-3.82 (2H, m), 7.69 (1H, dd, J=8.8, 2.5 Hz), 7.89 (1H, d, J=8.8 Hz), 8.21 (1H, s), 8.28 (1H, d, J=2.5 Hz).

### Reference Example 311

A mixture of sodium ethoxide (391 g) and diisopropyl ether (2 L) was added a mixture of diethyl succinate (500 g) and ethyl trifluoroacetate (836 g) at 60° C. over 3 hours. The reaction mixture was stirred overnight at 60° C. The reaction mixture was poured into ice water (2 L) and conc. hydrochloric acid was added to adjust to pH 2. The mixture was extracted with ethyl acetate. The ethyl acetate layer was washed with saturated brine, dried (MgSO₄) and concentrated to give an oily substance (796.2 g). A mixture of the obtained oily substance (796.2 g) and 40% aqueous sulfuric acid solution (3.3 L) was refluxed overnight. The reaction mixture was added to ice (2 kg), and extracted with ethyl acetate. The ethyl acetate layer was washed with saturated brine, dried (MgSO₄) and concentrated to give an oily substance (401.6 g). To a mixture of the obtained oily substance (401.6 g) and ethanol (1.5 L) was added hydrazine monohydrate (200 ml) at 0° C. and the mixture was refluxed overnight. The reaction mixture was concentrated and water was added to the residue. The mixture was extracted with ethyl acetate. The ethyl acetate layer was washed with saturated brine, dried (MgSO₄) and concentrated. The residue was subjected to silica gel column chromatography, and 4,5-dihydro-6-(trifluoromethyl)-3-pyridazinone (209.57 g, yield 44%) was obtained as yellow crystals from a fraction eluted with ethyl acetate-hexane (2:3, volume ratio). melting point: 94-95° C.

¹H-NMR (CDCl₃) δ: 2.57-2.85 (4H, m), 9.15 (1H, brs).

### Reference Example 312

A mixture of 4,5-dihydro-6-(trifluoromethyl)-3-pyridazinone (90.0 g), bromine (30.5 ml) and acetic acid (270 ml) was stirred at 80° C. for 1 hour. Ice water (500 ml) was added to the reaction mixture. The precipitated crystals were collected by filtration, washed with aqueous sodium hydrogen carbonate and water and dried to give 6-(trifluoromethyl)-3-pyridazinone (58.74 g, yield 66%) as white crystals. melting point: 129-130° C.

¹H-NMR (CDCl₃) δ: 7.14 (1H, dd, J=9.9, 0.5 Hz), 7.54 (1H, d, J=10.0 Hz), 12.64 (1H, brs).

### Reference Example 313

A mixture of 6-(trifluoromethyl)-3-pyridazinone (1.41 g), thionyl chloride (1.5 ml) and N,N-dimethylformamide (0.3 ml) was refluxed for 2 hours. Excess thionyl chloride was evaporated under reduced pressure and aqueous sodium hydrogen carbonate was added. The mixture was extracted with diethyl ether. The diethyl ether layer was washed with saturated brine, dried (MgSO₄) and concentrated. The residue was subjected to silica gel column chromatography, and 3-chloro-6-(trifluoromethyl)pyridazine (1.45 g, yield 92%) was obtained as white crystals from a fraction eluted with ethyl acetate-hexane (1:3, volume ratio). melting point: 51-52° C.

¹H-NMR (CDCl₃) δ: 7.75 (1H, dd, J=8.7, 0.6 Hz), 7.82 (1H, d, J=9.0 Hz).

### Reference Example 314

A mixture of 3-methyl-2-butanone (10.7 ml) and bis(dimethylamino)methoxymethane (6.61 g) was heated under reflux for 8 hours. The reaction mixture was concentrated under reduced pressure. Hydrazine monohydrate (5.80 g) and n-butyl alcohol (29 ml) were added to the residue and the mixture was heated under reflux for 6 hours. The reaction mixture was concentrated under reduced pressure. The residue was subjected to silica gel column chromatography and eluted with hexane-ethyl acetate (1:1, volume ratio) to give 3-isopropyl-1H-pyrazole (4.26 g, yield 59%) as a colorless oil.

¹H-NMR (CDCl₃) δ: 1.30 (6H, d, J=6.9 Hz), 2.84-3.24 (1H, m), 6.10 (1H, d, J=2.0 Hz), 7.49 (1H, d, J=1.9 Hz), 10.3 (1H, br s).

### Reference Example 315

In the same manner as in Reference Example 314, 3-(1-ethylpropyl)pyrazole (yield 91%) was obtained as a colorless oil.

¹H-NMR (CDCl₃) δ: 0.84 (6H, t, J=7.4 Hz), 1.5-1.8 (4H, m), 2.5-2.6 (1H, m), 6.06 (1H, d, J=1.9 Hz), 7.52 (1H, d, J=1.9 Hz).

### Reference Example 316

To a mixture of 3-isopropyl-1H-pyrazole (3.74 g), 2-chloro-5-trifluoromethylpyridine (6.17 g) and N-methylpyrrolidone (18.7 ml) was added NaOH (trademark: Tosoh pearl, 2.03 g) while stirring the mixture at room temperature. After reaction as it was for 9 hours, water (38 ml) and 6N hydrochloric acid (85 ml) were added, and the mixture was extracted with ethyl acetate. The extract was washed with water and concentrated under reduced pressure. The residue was subjected to silica gel column chromatography and eluted with hexane and then with toluene to give 2-(3-isopropyl-1H-pyrazol-1-yl)-5-(trifluoromethyl)pyridine (6.94 g, yield 80%) as a colorless oil.

¹H-NMR (CDCl₃) δ: 1.33 (6H, d, J=7.0 Hz), 3.0-3.2 (1H, m), 6.34 (1H, d, J=2.5 Hz), 7.97 (1H, dd, J=8.7, 2.1 Hz), 8.05 (1H, d, J=8.7 Hz), 8.47 (1H, d, J==2.5 Hz), 8.6-8.7 (1H, m).

### Reference Example 317

In the same manner as in Reference Example 316, 2-[3-(1-ethylpropyl)-1H-pyrazol-1-yl]-5-(trifluoromethyl)pyridine (yield 61%) was obtained as a colorless oil.

¹H-NMR (CDCl₃) δ: 0.87 (6H, t, J=7.4 Hz), 1.5-1.8 (4H, m), 2.6-2.7 (1H, m), 6.28 (1H, d, J=2.7 Hz), 7.97 (1H, dd, J=8.7, 2.2 Hz), 8.07 (1H, d, J=8.7 Hz), 8.49 (1H, d, J=2.7 Hz), 8.6-8.7 (1H, m).

### Reference Example 318

A solution of 2-(3-isopropyl-1H-pyrazol-1-yl)-5-(trifluoromethyl)pyridine (1.55 g) in acetonitrile (31 ml) was added iodine (924 mg), then diammonium cerium(IV) nitrate (2.00 g) while stirring the mixture at room temperature, and the mixture was reacted as it was for 5 hours. After the completion of the reaction, the reaction mixture was concentrated under reduced pressure. Water was added to the residue and the mixture was extracted with ethyl acetate. The organic layers were combined, washed with saturated aqueous sodium thiosulfate solution, dried (magnesium sulfate) and concentrated under reduced pressure to give 2-(4-iodo-3-isopropyl-1H-pyrazol-1-yl)-5-(trifluoromethyl)pyridine (2.19 g, yield 95%) as crystals.

¹H-NMR (CDCl₃) δ: 1.38 (6H, d, J=6.9 Hz), 3.0-3.2 (1H, m), 7.99 (1H, dd, J=8.7, 2.0 Hz), 8.05 (1H, d, J=8.7 Hz), 8.57 (1H, s), 8.6-8.7 (1H, m).

### Reference Example 319

In the same manner as in Reference Example 318, 2-[3-(1-ethylpropyl)-4-iodo-1H-pyrazol-1-yl]-5-(trifluoromethyl)pyridine (yield 95%) was obtained as a colorless oil.

¹H-NMR(CDCl₃) δ: 0.87 (6H, t, J=7.4 Hz), 1.6-1.9 (4H, m), 2.7-2.8 (1H, m), 7.99 (1H, dd, J=8.7, 2.1 Hz), 8.06 (1H, d, J=8.7 Hz), 8.59 (1H, s), 8.63 (1H, d, J=2.1 Hz).

### Reference Example 320

A mixture of 2-(4-iodo-3-isopropyl-1H-pyrazol-1-yl)-5-(trifluoromethyl)pyridine (841 mg), palladium acetate (49.6 mg), triphenylphosphine (116 mg), potassium acetate (434 mg), benzyltriethylammonium chloride (504 mg), methyl acrylate (0.793 ml) and N-methylpyrrolidone (8.41 ml) was stirred at room temperature under a nitrogen stream for 1 hour. The mixture was heated to outer temperature of 90° C. for 20 minutes and an insoluble material was filtered off and washed with ethyl acetate. Water was added to the filtrate, and the mixture was extracted with ethyl acetate. The organic layers were combined, washed with water and dried (magnesium sulfate). The mixture was concentrated under reduced pressure. The residue was subjected to silica gel column chromatography and eluted with hexane-ethyl acetate (95:5, volume ratio) to give methyl 3-{3-isopropyl-1-[5-(trifluoromethyl)pyridin-2-yl]-1H-pyrazol-4-yl}-2-propenoate (653 mg, yield 87%) as crystals.

¹H-NMR (CDCl₃) δ: 1.37 (6H, d, J=6.9 Hz), 3.1-3.3 (1H, m), 3.81 (3H, s), 6.29 (1H, d, J=16.0 Hz), 7.64 (1H, d, J=16.0 Hz), 8.00 (1H, dd, J=8.7, 2.1 Hz), 8.10 (1H, d, J=8.7 Hz), 8.6-8.7 (1H, m), 8.75 (1H, s).

### Reference Example 321

In the same manner as in Reference Example 320 except that ethyl acrylate was used instead of methyl acrylate, ethyl 3-{3-(2-ethylpropyl)-1-[5-(trifluoromethyl)pyridin-2-yl]-1H-pyrazol-4-yl}-2-propenoate (yield 70%) was obtained as a colorless oil.

¹H-NMR (CDCl₃) δ: 0.87 (6H, t, J=7.4 Hz), 1.34 (3H, t, J==7.1 Hz), 1.6-1.9 (4H, m), 2.7-2.8 (1H, m), 4.26 (2H, q, J=7.1 Hz), 6.31 (1H, d, J=16.0 Hz), 7.61 (1H, d, J=16.0 Hz), 8.01 (1H, dd, J=8.7, 2.2 Hz), 8.10 (1H, d, J=8.7 Hz), 8.6-8.7 (1H, m), 8.77 (1H, s).

### Reference Example 322

To a mixture of 3-isopropyl-1H-pyrazole (167 g), diammonium cerium(IV) nitrate (497 g) and acetonitrile (1200 ml) was added iodine (230 g) at 0° C. and the mixture was stirred overnight at room temperature. Water was added to the reaction mixture, and the mixture was extracted with ethyl acetate. The ethyl acetate layer was washed with saturated aqueous sodium thiosulfate solution and saturated brine, dried (MgSO₄) and concentrated to give 4-iodo-3-isopropyl-1H-pyrazole (254 g, yield 71%) as a dark brown oily substance.

¹H-NMR (CDCl₃) δ: 1.31 (6H, d, J=6.9 Hz), 3.00-3.17 (1H, m), 7.52 (1H, s).

### Reference Example 323

To a mixture of 4-iodo-3-isopropyl-1H-pyrazole (254 g), potassium tert-butoxide (156 g) and tetrahydrofuran (1000 ml) was added benzyl bromide (134 ml) and the mixture was stirred at 0° C. and at room temperature overnight. Water was added to the reaction mixture and the mixture was extracted with ethyl acetate. The ethyl acetate layer was washed with saturated brine, dried (MgSO₄) and concentrated. The residue was subjected to silica gel column chromatography, and 1-benzyl-4-iodo-3-isopropyl-1H-pyrazole (320 g, yield 92%) was obtained as a brown oily substance from a fraction eluted with ethyl acetate-hexane (1:9, volume ratio).

¹H-NMR (CDCl₃) δ: 1.30 (6H, d, J=6.9 Hz), 2.94-3.04 (1H, m) 5.24 (2H, s), 7.01-7.07 (1H, m), 7.16-7.36 (5H, m).

### Reference Example 324

To a mixture of 3-isopropyl-1H-pyrazole (92.5 g), potassium tert-butoxide (123 g) and tetrahydrofuran (840 ml) was added benzyl bromide (125 ml) at 0° C. and the mixture was stirred at room temperature for 5 hours. Water was added to the reaction mixture and the mixture was extracted with ethyl acetate. The ethyl acetate layer was washed with saturated brine, dried (MgSO₄) and concentrated. The residue was subjected to silica gel column chromatography, and 1-benzyl-3-isopropyl-1H-pyrazole (114 g, yield 68%) was obtained as a brown oily substance from a fraction eluted with ethyl acetate-hexane (1:9, volume ratio).

H-NMR (CDCl₃) δ: 1.27 (6H, d, J=7.2 Hz), 2.96-3.07 (1H, m), 5.26 (2H, s), 6.06-6.09 (1H, m), 7.02-7.07 (1H, m), 7.14-7.36 (5H, m).

### Reference Example 325

To a mixture of 1-benzyl-4-iodo-3-isopropyl-1H-pyrazole (110 g), palladium(II) acetate (7.56 g), triphenylphosphine (17.7 g), benzyltriethylammonium chloride (76.8 g), methyl acrylate (121 ml) and 1-methyl-2-pyrrolidone (1000 ml) was added sodium acetate (55.3 g) at room temperature and the mixture was stirred overnight at 80° C. under an argon atmosphere. The reaction mixture was cooled to room temperature and concentrated under reduced pressure. Ethyl acetate was added to the residue, and an insoluble material was removed by filtration. Water was added to the filtrate and the mixture was extracted with ethyl acetate. The extract was washed with dilute hydrochloric acid, saturated aqueous sodium thiosulfate solution and water, dried (MgSO₄) and concentrated. The residue was subjected to silica gel column chromatography, and methyl (E)-3-(1-benzyl-3-isopropyl-1H-pyrazol-4-yl)-2-propenoate (81.1 g, yield 81%) was obtained as a brown oily substance from a fraction eluted with ethyl acetate-hexane (1:7, volume ratio).

¹H-NMR (CDCl₃) δ: 1.32 (6H, d, J=6.9 Hz), 3.08-3.21 (1H, m), 3.75 (3H, s), 5.25 (2H, s), 6.02 (1H, d, J=16.2 Hz), 7.04-7.09 (1H, m), 7.20-7.38 (4H, m), 7.46 (1H, s), 7.59 (1H, d, J=16.2 Hz).

### Reference Example 326

To a mixture of methyl (E)-3-(1-benzyl-3-isopropyl-1H-pyrazol-4-yl)-2-propenoate (52.5 g), 5% palladium-carbon (100 g) and ethanol (500 ml) was added formic acid (250 ml), and the mixture was heated under reflux for 3 hours. The reaction mixture was cooled to room temperature and palladium-carbon was removed by filtration. The filtrate was concentrated and the residue was diluted with ethyl acetate. The obtained ethyl acetate solution was washed with saturated aqueous sodium hydrogen carbonate and saturated brine, dried (MgSO₄) and concentrated to give methyl 3-(3-isopropyl-1H-pyrazol-4-yl)propanoate (31.5 g, yield 87%) as a pale-yellow oily substance.

¹H-NMR (CDCl₃) δ: 1.29 (6H, d, J=7.2 Hz), 2.54-2.61 (2H, m) 2.74-2.82 (2H, m), 2.98-3.13 (1H, m), 3.68 (3H, s), 7.33 (1H, s).

### Reference Example 327

To a mixture of methyl 3-(3-isopropyl-1H-pyrazol-4-yl)propanoate (70.0 g), 3-chloro-6-(trifluoromethyl)pyridazine (71.6 g) and N,N-dimethylformamide (700 ml) was added sodium hydride (60% in oil, 16.4 g) at 0° C., and the mixture was stirred at said temperature for 2 hours. The reaction solution was poured into dilute hydrochloric acid and the organic layer was extracted with ethyl acetate. The extract was washed with dilute hydrochloric acid and saturated brine, dried (MgSO₄) and concentrated. The residue was subjected to silica gel column chromatography, and methyl 3-{3-isopropyl-1-[6-(trifluoromethyl)pyridazin-3-yl]-1H-pyrazol-4-yl}propanoate (92.6 g, yield 76%) was obtained as a pale-yellow solid from a fraction eluted with ethyl acetate-hexane (1:9, volume ratio).

¹H-NMR (CDCl₃) δ: 1.33 (6H, d, J=7.2 Hz), 2.64-2.71 (2H, m), 2.82-2.89 (2H, m), 3.00-3.10 (1H, m), 3.71 (3H, s), 7.84 (1H, d, J=9.0 Hz), 8.29 (1H, d, J=9.0 Hz), 8.49 (1H, s).

### Reference Example 328

To a solution of methyl 3-{3-isopropyl-1-[6-(trifluoromethyl)pyridazin-3-yl]-1H-pyrazol-4-yl)propanoate (92.6 g) in tetrahydrofuran (400 ml) was dropwise added a 1.5 M solution (396 ml) of diisobutylaluminum hydride in toluene at 0° C. and the mixture was stirred at said temperature for 30 minutes. Sodium sulfate 10 hydrate (87.0 g) was added to the reaction mixture at 0° C. and the mixture was stirred overnight at room temperature. Dilute hydrochloric acid was added to the mixture and the mixture was extracted with ethyl acetate. The extract was washed with dilute hydrochloric acid and saturated brine, dried (MgSO₄) and concentrated. The residue was subjected to silica gel column chromatography, and a pale-yellow solid was obtained from a fraction eluted with ethyl acetate-hexane (1:3, volume ratio). The obtained solid was washed with hexane to give 3-{3-isopropyl-1-[6-(trifluoromethyl)pyridazin-3-yl]-1H-pyrazol-4-yl}-1-propanol (57.8 g, yield 68%) as a white solid.

¹H-NMR(CDCl₃) δ: 1.33 (6H, d, J=6.6 Hz), 1.42 (1H, t, J=5.1 Hz), 1.84-2.01 (2H, m), 2.63 (2H, t, J=7.9 Hz), 3.05 (1H, septet, J=6.8 Hz), 3.77 (2H, q, J=5.7 Hz), 7.83 (1H, d, J=9.0 Hz), 8.29 (1H, d, J=9.0 Hz), 8.50 (1H, s).

### Reference Example 329

To a solution of 4-(benzyloxy)-2-hydroxybenzaldehyde (16.5 g) in ethylene glycol (90 ml) were added potassium hydroxide (12.2 g) and hydrazine monohydrate (10.6 ml) at room temperature and the mixture was stirred at 120° C. for 3 hours and at 199° C. overnight. The reaction solution was cooled to room temperature and 2N hydrochloric acid (110 ml) was added. The mixture was extracted with ethyl acetate. The extract was washed with water and saturated brine, and dried (MgSO₄) and concentrated to give 5-(benzyloxy)-2-methylphenol (14.8 g, yield 95%) as a brown oily substance.

¹H-NMR (CDCl₃) δ: 2.17 (3H, s), 5.01 (2H, s), 6.43-6.51 (2H, m), 6.99 (1H, d, J=8.4 Hz), 7.27-7.43 (5H, m).

### Reference Example 330

To a mixture of 5-(benzyloxy)-2-methylphenol (14.8 g), methyl chloromethyl ether (7.82 ml) and tetrahydrofuran (250 ml) was added sodium hydride (60% in oil, 4.12 g) at 0° C. and the mixture was stirred at room temperature for 5 hours. Water was added to the reaction mixture and the mixture was extracted with ethyl acetate. The extract was washed with saturated brine, dried (MgSO₄) and concentrated. The residue was subjected to silica gel column chromatography, and 4-(benzyloxy)-2-(methoxymethoxy)-1-methylbenzene (12.8 g, yield 72%) was obtained as a colorless oil from a fraction eluted with ethyl acetate-hexane (1:19, volume ratio).

¹H-NMR (CDCl₃) δ: 2.17 (3H, s), 3.47 (3H, s), 5.02 (2H, s), 5.16 (2H, s), 6.53 (1H, dd, J=2.4, 8.4 Hz), 6.75 (1H, d, J=2.4 Hz), 7.02 (1H, d, J=8.4 Hz), 7.28-7.45 (5H, m).

### Reference Example 331

A mixture of 4-(benzyloxy)-2-(methoxymethoxy)-1-methylbenzene (12.8 g), 5% palladium-carbon (2.56 g) and ethanol (200 ml) was stirred overnight at room temperature under a hydrogen atmosphere. Palladium-carbon was removed by filtration and the filtrate was concentrated to give 3-(methoxymethoxy)-4-methylphenol (7.98 g, yield 96%) as a colorless oil.

¹H-NMR (CDCl₃) δ: 2.15 (3H, s), 3.48 (3H, s), 5.16 (2H, s) 6.39 (1H, dd, J=2.4, 8.1 Hz), 6.60 (1H, d, J=2.4 Hz), 6.96 (1H, d, J=8.1 Hz).

### Reference Example 332

A mixture of 3-(methoxymethoxy)-4-methylphenol (7.98 g), ethyl 2-bromoisobutyrate (50 ml), potassium carbonate (48.7 g) and N,N-dimethylformamide (200 ml) was stirred at 80° C. for 2 hours. Saturated aqueous ammonium chloride solution was added to the reaction mixture, and the mixture was extracted with ethyl acetate. The extract was washed with saturated aqueous ammonium chloride solution and saturated brine, dried (MgSO₄) and concentrated. The residue was subjected to silica gel column chromatography, and ethyl 2-[3-(methoxymethoxy)-4-methylphenoxy]-2-methylpropanoate (10.6 g, yield 79%) was obtained as a colorless oil from a fraction eluted with ethyl acetate-hexane (1:19, volume ratio).

¹H-NMR (CDCl₃) δ: 1.27 (3H, t, J=6.9 Hz), 1.57 (6H, s), 2.16 (3H, s), 3.46 (3H, s), 4.23 (2H, q, J=6.9 Hz), 5.13 (2H, s), 6.36 (1H, dd, J=2.4, 8.1 Hz), 6.65 (1H, d, J=2.4 Hz), 6.95 (1H, d, J=8.1 Hz).

### Reference Example 333

To a solution of ethyl 2-[3-(methoxymethoxy)-4-methylphenoxy]-2-methylpropanoate (10.6 g) in ethanol (150 ml) was added several drops of conc. hydrochloric acid, and the mixture was stirred while heating under reflux for 4 hours. The reaction solution was cooled to room temperature, and concentrated. The residue was subjected to silica gel column chromatography, and ethyl 2-(3-hydroxy-4-methylphenoxy)-2-methylpropanoate (7.56 g, yield 85%) was obtained as a colorless oil from a fraction eluted with ethyl acetate-hexane (3:17, volume ratio).

¹H-NMR (CDCl₃) δ: 1.26 (3H, t, J=7.2 Hz), 1.57 (6H, s), 2.16 (3H, s), 4.23 (2H, q, J=7.2 Hz), 4.77 (1H, s), 6.30-6.37 (2H, m), 6.93 (1H, d, J=7.8 Hz).

### Reference Example 334

To a mixture of 2′,4′-dihydroxyacetophenone (25.0 g), potassium carbonate (24.9 g) and acetone (500 ml) was dropwise added benzyl bromide (21.4 ml) at 0° C. and the mixture was stirred overnight at room temperature. The insoluble material was removed by filtration and the filtrate was concentrated to give a pale-yellow solid. The obtained solid was recrystallized from ethanol to give 1-[4-(benzyloxy)-2-hydroxyphenyl]ethanone (33.8 g, yield 85%) as colorless crystals. melting point: 107-108° C.

### Reference Example 335

To a solution of 1-[4-(benzyloxy)-2-hydroxyphenyl]ethanone (32.8 g) in tetrahydrofuran (400 ml) were added methyl chloromethyl ether (24.8 ml) and potassium tert-butoxide (36.6 g) at 0° C. and the mixture was stirred overnight at room temperature. Water was added to the reaction mixture and the mixture was extracted with ethyl acetate. The extract was washed with saturated brine, dried (MgSO₄) and concentrated. The residue was subjected to silica gel column chromatography, and 1-[4-(benzyloxy)-2-(methoxymethoxy)phenyl]ethanone (18.6 g, yield 48%) was obtained as a colorless oil from a fraction eluted with ethyl acetate-hexane (1:4, volume ratio).

¹H-NMR (CDCl₃) δ: 2.61 (3H, s), 3.51 (3H, s), 5.10 (2H, s), 5.26 (2H, s), 6.65 (1H, dd, J=2.2, 8.8 Hz), 6.79 (1H, d, J-2.2 Hz), 7.32-7.48 (5H, m), 7.81 (1H, d, J=8.8 Hz).

### Reference Example 336

To a solution of 1-[4-(benzyloxy)-2-(methoxymethoxy)phenyl]ethanone (10.0 g) in ethylene glycol (50 ml) were added potassium hydroxide (5.88 g) and hydrazine monohydrate (5.11 ml) at room temperature and the mixture was stirred at 120° C. for 2 hours and at 199° C. overnight. The reaction solution was cooled to room temperature, neutralized with 2N hydrochloric acid, and extracted with ethyl acetate. The extract was washed with saturated brine, dried (MgSO₄) and concentrated. The residue was subjected to silica gel column chromatography, and 5-(benzyloxy)-2-ethylphenol (4.43 g, yield 56%) was obtained as a yellow oily substance from a fraction eluted with ethyl acetate-hexane (1:9, volume ratio).

¹H-NMR (CDCl₃) δ: 1.21 (3H, t, J=7.5 Hz), 2.56 (2H, q, J=7.5 Hz), 4.68 (1H, s), 5.01 (2H, s), 6.44 (1H, d, J=2.4 Hz), 6.51 (1H, dd, J=2.4, 8.4 Hz), 7.01 (1H, d, J=8.4 Hz), 7.27-7.44 (5H, m).

### Reference Example 337

To a solution of 3-(benzyloxy)-4-methoxybenzaldehyde (10.0 g) in methylene chloride (200 ml) was added m-chloroperbenzoic acid (24.4 g) at 0° C. and the mixture was stirred at said temperature for 2 hours. To a reaction solution was added a saturated aqueous sodium thiosulfate solution, and the mixture was extracted with ethyl acetate. The extract was washed with saturated aqueous sodium hydrogen carbonate and saturated brine, dried (MgSO₄) and concentrated. A mixture of the residue, a 2N ammonia-methanol solution (100 ml) and methanol (100 ml) was stirred overnight at room temperature. The reaction mixture was concentrated. The residue was subjected to silica gel column chromatography and 3-(benzyloxy)-4-methoxyphenol (8.78 g, yield 92%) was obtained as a pale-yellow oily substance from a fraction eluted with ethyl acetate-hexane (1:4, volume ratio).

¹H-NMR (CDCl₃) δ: 3.84 (3H, s), 4.51 (1H, s), 5.12 (2H, s), 6.35 (1H, dd, J=3.0, 8.8 Hz), 6.47 (1H, d, J=3.0 Hz), 6.76 (1H, d, J=8.8 Hz), 7.28-7.50 (5H, m).

### Reference Example 338

To a solution of 5-(benzyloxy)-2-ethylphenol (4.43 g) in tetrahydrofuran (90 ml) was added sodium hydride (60% in oil, 1.16 g) at room temperature and the mixture was stirred for 30 minutes. Methyl chloromethyl ether (2.21 ml) was added at room temperature, and the mixture was stirred overnight. Water was added to the reaction mixture and the mixture was extracted with ethyl acetate. The extract was washed with saturated brine, dried (MgSO₄) and concentrated. The residue was subjected to silica gel column chromatography, and 4-(benzyloxy)-1-ethyl-2-(methoxymethoxy)benzene (4.57 g, yield 86%) was obtained as a colorless oil from a fraction eluted with ethyl acetate-hexane (1:19, volume ratio).

¹H-NMR (CDCl₃) δ: 1.17 (3H, t, J=7.4 Hz), 2.59 (2H, q, J=7.4 Hz), 3.48 (3H, s), 5.02 (2H, s), 5.17 (2H, s), 6.56 (1H, dd, J=2.6, 8.4 Hz), 6.77 (1H, d, J=2.6 Hz), 7.05 (1H, d, J=8.4 Hz), 7.30-7.48 (5H, m).

### Reference Example 339

A mixture of 3-(benzyloxy)-4-methoxyphenol (8.78 g), ethyl 2-bromoisobutyrate (28.0 ml), potassium carbonate (26.3 g) and N,N-dimethylformamide (190 ml) was stirred at 80° C. for 5 hours. Saturated aqueous ammonium chloride solution was added to the reaction mixture, and the mixture was extracted with ethyl acetate. The extract was washed with saturated brine, dried (MgSO₄) and concentrated. The residue was subjected to silica gel column chromatography, and ethyl 2-[3-(benzyloxy)-4-methoxyphenoxy]-2-methylpropanoate (11.0 g, yield 83%) was obtained as a pale-yellow oily substance from a fraction eluted with ethyl acetate-hexane (1:9, volume ratio).

¹H-NMR (CDCl₃) δ: 1.25 (3H, t, J=7.0 Hz), 1.47 (6H, s), 3.84 (3H, s), 4.18 (2H, q, J=7.0 Hz), 5.10 (2H, s), 6.41 (1H, dd, J=2.6, 8.8 Hz), 6.54 (1H, d, J=2.6 Hz), 6.74 (1H, d, J=8.8 Hz), 7.24-7.46 (5H, m).

### Reference Example 340

A mixture of 4-(benzyloxy)-1-ethyl-2-(methoxymethoxy)benzene (4.57 g), 5% palladium-carbon (1.00 g) and ethanol (90 ml) was stirred overnight at room temperature under a hydrogen atmosphere. Palladium-carbon was removed by filtration and the filtrate was concentrated to give 4-ethyl-3-(methoxymethoxy)phenol (3.06 g, quantitative) as a pale-yellow oily substance.

¹H-NMR (CDCl₃) δ: 1.16 (3H, t, J=7.4 Hz), 2.58 (2H, q, J=7.4 Hz), 3.48 (3H, s), 4.69 (1H, s), 5.17 (2H, s), 6.42 (1H, dd, J=2.6, 8.0 Hz), 6.62 (1H, d, J=2.6 Hz), 7.00 (1H, d, J=8.0 Hz).

### Reference Example 341

A mixture of ethyl 2-[3-(benzyloxy)-4-methoxyphenoxy]-2-methylpropanoate (11.0 g), 5% palladium-carbon (2.19 g) and ethanol (160 ml) was stirred overnight at room temperature under a hydrogen atmosphere. Palladium-carbon was removed by filtration and the filtrate was concentrated. The residue was subjected to silica gel column chromatography, and ethyl 2-(3-hydroxy-4-methoxyphenoxy)-2-methylpropanoate (8.00 g, yield 99%) was obtained as a colorless oil from a fraction eluted with ethyl acetate.

¹H-NMR (CDCl₃) δ: 1.29 (3H, t, J=7.2 Hz), 1.54 (6H, s), 3.84 (3H, s), 4.24 (2H, q, J=7.2 Hz), 5.57 (1H, s), 6.37 (1H, dd, J=3.0, 8.7 Hz), 6.54 (1H, d, J=3.0 Hz), 6.69 (1H, d, J=8.7 Hz).

### Reference Example 342

A mixture of 4-ethyl-3-(methoxymethoxy)phenol (3.06 g), ethyl 2-bromoisobutyrate (9.86 ml), potassium carbonate (9.28 g) and N,N-dimethylformamide (85 ml) was stirred overnight at BOOC. Saturated aqueous ammonium chloride solution was added to the reaction mixture, and the mixture was extracted with ethyl acetate. The extract was washed with saturated brine, dried (MgSO₄) and concentrated. The residue was subjected to silica gel column chromatography, and ethyl 2-[4-ethyl-3-(methoxymethoxy)phenoxy]-2-methylpropanoate (4.93 g, yield 99%) was obtained as a colorless oil from a fraction eluted with ethyl acetate-hexane (1:19, volume ratio).

¹H-NMR (CDCl₃) δ: 1.16 (3H, t, J=7.4 Hz), 1.27 (3H, t, J=7.4 Hz), 1.58 (6H, s), 2.57 (2H, q, J=7.4 Hz), 3.46 (3H, s), 4.24 (2H, q, J=7.4 Hz), 5.14 (2H, s), 6.39 (1H, dd, J=2.6, 8.0 Hz), 6.66 (1H, d, J=2.6 Hz), 6.97 (1H, d, J=8.0 Hz).

### Reference Example 343

To a solution of ethyl 2-[4-ethyl-3-(methoxymethoxy)phenoxy]-2-methylpropanoate (4.93 g) in ethanol (85 ml) was added several drops of conc. hydrochloric acid and the mixture was stirred overnight while heating under reflux. The reaction solution was cooled to room temperature and concentrated. The residue was subjected to silica gel column chromatography, and ethyl 2-(4-ethyl-3-hydroxyphenoxy)-2-methylpropanoate (3.72 g, yield 89%) was obtained as a pale-yellow oily substance from a fraction eluted with ethyl acetate-hexane (3:37, volume ratio).

¹H-NMR (CDCl₃) δ: 1.20 (3H, t, J=7.5 Hz), 1.26 (3H, t, J=7.2 Hz), 1.57 (6H, s), 2.55 (2H, q, J=7.5 Hz), 4.24 (2H, q, J=7.2 Hz), 4.75 (1H, s), 6.33-6.39 (2H, m), 6.96 (1H, d, J=8.1 Hz).

### Reference Example 344

To a mixture of 1-benzyl-3-isopropyl-1H-pyrazole (224 g), diammonium cerium(IV) nitrate (368 g) and acetonitrile (1000 ml) was added iodine (171 g) at 0° C. and the mixture was stirred overnight at room temperature. Water was added to the reaction mixture and the mixture was extracted with ethyl acetate. The ethyl acetate layer was washed with saturated aqueous sodium thiosulfate solution and saturated brine, dried (MgSO₄) and concentrated. The residue was subjected to silica gel column chromatography, and 1-benzyl-4-iodo-3-isopropyl-1H-pyrazole (340 g, yield 93%) was obtained as a brown oily substance from a fraction eluted with ethyl acetate-hexane (1:9, volume ratio).

¹H-NMR (CDCl₃) δ: 1.30 (6H, d, J=6.9 Hz), 2.94-3.04 (1H, m) 5.24 (2H, s), 7.01-7.07 (1H, m), 7.16-7.36 (5H, m).

### Reference Example 345

A mixture of 2-{3-(1-ethylpropyl)-4-iodo-1H-pyrazol-1-yl}-5-(trifluoromethyl)pyridine (4.09 g), palladium acetate (112 mg), triphenylphosphine (262 mg), sodium carbonate (2.12 g), benzyltriethylammonium chloride (2.28 g), allyl alcohol (1.02 ml), water (4.09 ml) and N,N-dimethylformamide (40.9 ml) was stirred at room temperature for 1 hour under a nitrogen stream. The mixture was heated at an outer temperature of 60° C. for 8 hours. The insoluble material was filtered off and washed with ethyl acetate. Water was added to the filtrate and the mixture was extracted with ethyl acetate. The organic layers were combined, washed with saturated aqueous sodium thiosulfate solution, dried (Na₂SO₄) and concentrated under reduced pressure. The residue was subjected to silica gel column chromatography and eluted with hexane-ethyl acetate (9:1, volume ratio) to give 3-[3-(1-ethylpropyl)-1-[5-(trifluoromethyl)pyridin-2-yl]-1H-pyrazol-4-yl}propanal (1.17 g, yield 35%) as a colorless oil.

¹H-NMR (CDCl₃) δ: 0.7-0.9 (6H, m), 1.6-1.9 (4H, m), 2.5-2.6 (1H, m), 2.7-2.8 (4H, m), 7.9-8.1 (2H, m), 8.27 (1H, s), 8.5-8.6 (1H, m), 9.86 (1H, s).

### Reference Example 346

To a solution of 3-{3-(1-ethylpropyl)-1-[5-(trifluoromethyl)pyridin-2-yl]-1H-pyrazol-4-yl}propanal (1.15 g) in methanol (25.2 ml) was added sodium borohydride (492 mg) with stirring under ice-cooling under a nitrogen stream. After stirring at said temperature for 0.5 hour, water (50 ml) and 6N hydrochloric acid (13 mmol) were added, and the mixture was stirred for 1 hour. The mixture was neutralized with 2N aqueous sodium hydroxide solution and extracted with ethyl acetate. The organic layers were combined, washed with water, dried (Na₂SO₄) and concentrated under reduced pressure. The residue was subjected to silica gel column chromatography and eluted with hexane-ethyl acetate (4:1, volume ratio) to give 3-{3-(1-ethylpropyl)-1-[5-(trifluoromethyl)pyridin-2-yl]-1H-pyrazol-4-yl}propan-1-ol (669 mg, yield 58%) as a colorless oil.

¹H-NMR (CDCl₃) δ: 0.87 (6H, t, J=7.4 Hz), 1.6-1.9 (6H, m), 2.5-2.7 (3H, m), 3.6-3.8 (2H, m), 7.94 (1H, dd, J=8.7, 2.2 Hz), 8.03 (1H, d, J=8.7 Hz), 8.28 (1H, s), 8.5-8.6 (1H, m).

### Reference Example 347

A mixture of 2-(4-iodo-3-isopropyl-1H-pyrazol-1-yl)-5-(trifluoromethyl)pyridine (7.62 g), palladium acetate (225 mg), triphenylphosphine (525 mg), sodium hydrogen carbonate (3.28 g), benzyltriethylammonium chloride (4.56 g), allyl alcohol (2.05 ml), water (7.62 ml) and N-methylpyrrolidone (76.2 ml) was stirred at room temperature for 1 hour under a nitrogen stream. The mixture was heated at an outer temperature of 60° C. for 6 hours. The insoluble material was filtered off and washed with ethyl acetate. Water was added to the filtrate and the mixture was extracted with ethyl acetate. The organic layers were combined, washed with saturated aqueous sodium thiosulfate solution, dried (Na₂SO₄) and concentrated under reduced pressure. The residue was subjected to silica gel column chromatography and eluted with hexane-ethyl acetate (9:1, volume ratio) to give 3-{3-isopropyl-1-[5-(trifluoromethyl)pyridin-2-yl]-1H-pyrazol-4-yl}propanal (3.93 g, yield 63%) as a colorless oil.

¹H-NMR (CDCl₃) δ: 1.34 (6H, d, J=6.9 Hz), 2.7-3.1 (5H, m), 7.95 (1H, dd, J=8.7, 2.2 Hz), 8.03 (1H, d, J=8.7 Hz), 8.26 (1H, s), 8.60 (1H, d, J=2.0 Hz), 9.86 (1H, s).

### Reference Example 348

To a solution of 3-{3-isopropyl-1-[5-(trifluoromethyl)pyridin-2-yl]-1H-pyrazol-4-yl}propanal (3.89 g) in methanol (77.8 ml) was added sodium borohydride (1.66 g) with stirring under ice-cooling under a nitrogen stream. After stirring at said temperature for 1 hour, water (50 ml) and 6N hydrochloric acid (44 mmol) were added, and the mixture was stirred for 1 hour. The precipitated crystals were collected by filtration to give 3-{3-isopropyl-1-[5-(trifluoromethyl)pyridin-2-yl]-1H-pyrazol-4-yl}propan-1-ol (3.73 g, yield 95%).

¹H-NMR (CDCl₃) δ: 1.33 (6H, d, J=6.9 Hz), 1.8-2.0 (2H, m), 2.60 (2H, t, J=7.9 Hz), 3.0-3.1 (1H, m), 3.75 (2H, t, J=6.3 Hz), 7.94 (1H, dd, J=8.7, 2.2 Hz), 8.03 (1H, d, J=8.7 Hz), 8.28 (1H, s), 8.6-8.7 (1H, m).

### Reference Example 349

To a mixture of 3-{3-isopropyl-1-[5-(trifluoromethyl)pyridin-2-yl]-1H-pyrazol-4-yl}propan-1-ol (470 mg) and toluene (9.4 ml) were added triethylamine (258 mg) and then methanesulfonyl chloride (258 mg) with stirring under ice-cooling. After stirring at room temperature for 30 minutes, water (10 ml) was added, and the mixture was extracted with toluene. The organic layer was washed with saturated brine and the mixture was concentrated under reduced pressure. o-Vanillin (342 mg), potassium carbonate (353 mg), ethanol (4.7 ml) and toluene (4.7 ml) were added to the residue and the mixture was reacted under reflux for 5.5 hours. After completion of the reaction, water (10 ml) was added to the reaction mixture and the mixture was extracted with ethyl acetate. The organic layer was washed with 3N aqueous sodium hydroxide solution and saturated aqueous sodium hydrogen carbonate in this order, dried over anhydrous sodium sulfate and concentrated under reduced pressure. The residue was subjected to silica gel column chromatography and eluted with hexane-ethyl acetate (9:1, volume ratio) to give 2-(3-{3-isopropyl-1-[5-(trifluoromethyl)pyridin-2-yl]-1H-pyrazol-4-yl}propoxy)-3-methoxybenzaldehyde (450 mg, yield 67%) as colorless crystals.

¹H-NMR (CDCl₃) δ: 1.34 (6H, d, J=6.9 Hz), 2.1-2.2 (2H, m), 2.73 (2H, t, J=7.7 Hz), 3.0-3.1 (1H, m), 3.90 (3H, s), 4.22 (2H, t, J=6.3 Hz), 7.1-7.2 (2H, m), 7.4-7.5 (1H, m), 7.95 (1H, dd, J=8.7, 2.2 Hz), 8.04 (1H, d, J=8.7 Hz), 8.33 (1H, s), 8.6-8.7 (1H, m), 10.5 (1H, s).

### Reference Example 350

To a mixture of 3-{3-isopropyl-1-[5-(trifluoromethyl)pyridin-2-yl]-1H-pyrazol-4-yl}propan-1-ol (470 mg) and tetrahydrofuran (13.8 ml) were added triethylamine (0.927 ml) and then methanesulfonyl chloride (0.511 ml) with stirring under ice-cooling. After stirring under ice-cooling for 1.5 hours, water was added, and the mixture was extracted with ethyl acetate. The organic layer was washed with saturated brine and concentrated under reduced pressure. o-Vanillin (1.21 g), potassium carbonate (1.09 g), acetonitrile (27.6 ml) were added to the residue and the mixture was reacted under reflux for 2.5 hours. After completion of the reaction, water was added to the reaction mixture and the mixture was extracted with ethyl acetate. The organic layer was washed with 3N aqueous sodium hydroxide solution and water in this order, dried over anhydrous sodium sulfate and concentrated under reduced pressure. Hexane was added to the residue to give 2-(3-{3-isopropyl-1-[5-(trifluoromethyl)pyridin-2-yl]-1H-pyrazol-4-yl}propoxy)-3-methoxybenzaldehyde (1.21 g) as crystals. The mother liquor was concentrated, subjected to silica gel column chromatography and eluted with hexane-ethyl acetate (9:1, volume ratio) to give 2-(3-{3-isopropyl-1-[5-(trifluoromethyl)pyridin-2-yl]-1H-pyrazol-4-yl}propoxy)-3-methoxybenzaldehyde (401 mg, total yield 77%) as colorless crystals.

¹H-NMR (CDCl₃) δ: 1.34 (6H, d, J=6.9 Hz), 2.1-2.2 (2H, m), 2.73 (2H, t, J=7.7 Hz), 3.0-3.1 (1H, m), 3.90 (3H, s), 4.22 (2H, t, J=6.3 Hz), 7.1-7.2 (2H, m), 7.4-7.5 (1H, m), 7.95 (1H, dd, J=8.7, 2.2 Hz), 8.04 (1H, d, J=8.7 Hz), 8.33 (1H, s), 8.6-8.7 (1H, m), 10.5 (1H, s).

### Reference Example 351

In the same manner as in Reference Example 350, 2-(3-{3-(1-ethylpropyl)-1-[5-(trifluoromethyl)pyridin-2-yl]-1H-pyrazol-4-yl}propoxy)-3-methoxybenzaldehyde (yield 67%) was obtained.

¹H-NMR (CDCl₃) δ: 1.34 (6H, t, J=7.4 Hz), 1.5-1.8 (4H, m), 2.0-2.2 (2H, m), 2.3-2.8 (3H, m), 3.90 (3H, s), 4.1-4.3 (2H, s), 7.1-7.2 (2H, m), 7.4-7.5 (1H, m), 7.90-8.00 (2H, m), 8.33 (1H, s), 8.6-8.7 (1H, m), 10.5 (1H, s).

### Example 1

A mixture of 3-{3-[4-(trifluoromethyl)phenyl]-5-isoxazolyl}-1-propyl methanesulfonate (1.04 g), sodium iodide (450 mg), methyl 4-hydroxyphenylacetate (500 mg), potassium carbonate (440 mg) and N,N-dimethylformamide (10 ml) was stirred at 90° C. for 5 hours. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours. 1N Hydrochloric acid (5 ml) was added and the mixture was extracted with ethyl acetate. The ethyl acetate layer was washed with saturated aqueous sodium chloride solution, dried (MgSO₄) and concentrated. The obtained colorless crystals were collected by filtration to give [4-(3-{3-[4-(trifluoromethyl)phenyl]-5-isoxazolyl}propoxy)phenyl]acetic acid (300 mg, yield 25%). The crystals were recrystallized from ethyl acetate-hexane. melting point: 127-128° C.

¹H-NMR (CDCl₃)δ:2.18-2.32 (2H, m), 2.98-3.10 (2H, m), 3.60 (2H, s), 3.98-4.08 (2H, m), 6.37 (1H, s), 6.82-6.90 (2H, m), 7.15-7.24 (2H, m), 7.66-7.75 (2H, m), 7.86-7.94 (2H, m).

### Example 2

A mixture of 3-{3-[4-(trifluoromethyl)phenyl]-5-isoxazolyl}-1-propyl methanesulfonate (1.04 g), sodium iodide (450 mg), methyl 4-hydroxybenzoate (460 mg), potassium carbonate (450 mg) and N,N-dimethylformamide (10 ml) was stirred at 90° C. for 5 hours. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours. 1N Hydrochloric acid (5 ml) was added and the mixture was extracted with ethyl acetate. The ethyl acetate layer was washed with saturated aqueous sodium chloride solution, dried (MgSO₄) and concentrated. The obtained colorless crystals were collected by filtration to give 4-(3-{3-[4-(trifluoromethyl)phenyl]-5-isoxazolyl}propoxy)benzoic acid (840 mg, yield 72%). The crystals were recrystallized from acetone-hexane. melting point: 221-222° C.

¹H-NMR (CDCl₃)δ: 2.20-2.38 (2H, m), 3.00-3.14 (2H, m), 4.05-4.18 (2H, m), 6.39 (1H, s), 6.86-6.96 (2H, m), 7.64-7.74 (2H, m), 7.86-8.08 (4H, m).

### Example 3

A mixture of 3-{3-[4-(trifluoromethyl)phenyl]-5-isoxazolyl}-1-propyl methanesulfonate (1.04 g), sodium iodide (450 mg), methyl 3-hydroxyphenylacetate (500 mg), potassium carbonate (450 mg) and N,N-dimethylformamide (10 ml) was stirred at 90° C. for 5 hours. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours. 1N Hydrochloric acid (5 ml) was added and the mixture was extracted with ethyl acetate. The ethyl acetate layer was washed with saturated aqueous sodium chloride solution, dried (MgSO₄) and concentrated. The obtained colorless crystals were collected by filtration to give [3-(3-{3-[4-(trifluoromethyl)phenyl]-5-isoxazolyl}propoxy)phenyl]acetic acid (630 mg, yield 52%). The crystals were recrystallized from ethyl acetate-hexane. melting point: 126-127° C.

¹H-NMR (CDCl₃)δ: 2.16-2.34 (2H, m), 2.98-3.12 (2H, m), 3.63 (2H, s), 4.00-4.10 (2H, m), 6.38 (1H, s), 6.76-6.94 (3H, m), 7.18-7.32 (1H, m), 7.66-7.75 (2H, m), 7.86-7.96 (2H, m).

### Example 4

A mixture of 3-{3-[4-(trifluoromethyl)phenyl]-5-isoxazolyl}-1-propyl methanesulfonate (1.04 g), sodium iodide (520 mg), methyl 3-hydroxybenzoate (460 mg), potassium carbonate (450 mg) and N,N-dimethylformamide (10 ml) was stirred at 90° C. for 5 hours. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours. 1N Hydrochloric acid (5 ml) was added and the mixture was extracted with ethyl acetate. The ethyl acetate layer was washed with saturated aqueous sodium chloride solution, dried (MgSO₄) and concentrated. The obtained colorless crystals were collected by filtration to give 3-(3-{3-[4-(trifluoromethyl)phenyl]-5-isoxazolyl}propoxy)benzoic acid (860 mg, yield 74%). The crystals were recrystallized from ethyl acetate-hexane. melting point: 133-134° C.

¹H-NMR (CDCl₃)δ: 2.20-2.37 (2H, m), 3.02-3.14 (2H, m), 4.06-4.17 (2H, m), 6.39 (1H, s), 7.10-7.20 (1H, m), 7.34-7.44 (1H, m), 7.58-7.76 (4H, m), 7.86-7.96 (2H, m).

### Example 5

A mixture of 3-{3-[4-(trifluoromethyl)phenyl]-5-isoxazolyl}-1-propyl methanesulfonate (1.04 g), sodium iodide (520 mg), ethyl 3-(4-hydroxyphenyl)propionate (600 mg), potassium carbonate (450 mg) and N,N-dimethylformamide (10 ml) was stirred at 90° C. for 5 hours. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and ethanol (5 ml) was stirred at room temperature for 5 hours. 1N Hydrochloric acid (5 ml) was added and extracted with ethyl acetate. The ethyl acetate layer was washed with saturated aqueous sodium chloride solution, dried (MgSO₄) and concentrated. The obtained colorless crystals were collected by filtration to give 3-[4-(3-{3-[4-(trifluoromethyl)phenyl]-5-isoxazolyl}propoxy)phenyl]propionic acid (520 mg, yield 42%). The crystals were recrystallized from ethyl acetate-hexane. melting point: 174-175° C.

¹H-NMR (CDCl₃)δ: 2.16-2.34 (2H, m), 2.59-2.72 (2H, m), 2.84-3.12 (4H, m), 3.98-4.08 (2H, m), 6.37 (1H, s), 6.78-6.88 (2H, m), 7.07-7.18 (2H, m), 7.66-7.76 (2H, m), 7.86-7.96 (2H, m).

### Example 6

A mixture of 3-{3-[4-(trifluoromethyl)phenyl]-5-isoxazolyl}-1-propyl methanesulfonate (1.04 g), sodium iodide (500 mg), methyl salicylate (460 mg), potassium carbonate (500 mg) and N,N-dimethylformamide (10 ml) was stirred at 90° C. for 5 hours. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours. 1N Hydrochloric acid (5 ml) was added, and extracted with ethyl acetate. The ethyl acetate layer was washed with saturated aqueous sodium chloride solution, dried (MgSO₄) and concentrated. The obtained colorless crystals were collected by filtration to give 2-(3-{3-[4-(trifluoromethyl)phenyl]-5-isoxazolyl}propoxy)benzoic acid (710 mg, yield 61%). The crystals were recrystallized from ethyl acetate-hexane. melting point: 132-133° C.

¹H-NMR (CDCl₃)δ: 2.34-2.52 (2H, m), 3.03-3.16 (2H, m), 4.18-4.42 (2H, m), 6.43 (1H, s), 7.00-7.24 (2H, m), 7.50-7.64 (1H, m), 7.65-7.76 (2H, m), 7.85-7.96 (2H, m), 8.16-8.24 (1H, m).

### Example 7

A mixture of 3-{3-[4-(trifluoromethyl)phenyl]-5-isoxazolyl}-1-propyl methanesulfonate (1.04 g), sodium iodide (500 mg), methyl 3-hydroxy-1-methyl-1H-pyrazole-5-carboxylate (470 mg), potassium carbonate (500 mg) and N,N-dimethylformamide (10 ml) was stirred at 90° C. for 5 hours. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours. 1N Hydrochloric acid (5 ml) was added, and extracted with ethyl acetate. The ethyl acetate layer was washed with saturated aqueous sodium chloride solution, dried (MgSO₄) and concentrated. The obtained colorless crystals were collected by filtration to give 1-methyl-3-(3-{3-[4-(trifluoromethyl)phenyl]-5-isoxazolyl}propoxy)-1H-pyrazole-5-carboxylic acid (870 mg, yield 74%). The crystals were recrystallized from ethyl acetate-hexane. melting point: 162-163° C.

¹H-NMR (CDCl₃)δ: 2.16-2.34 (2H, m), 2.96-3.10 (2H, m), 4.04 (3H, s), 4.17-4.28 (2H, m), 6.30 (1H, s), 6.39 (1H, s), 7.67-7.77 (2H, m), 7.87-7.97 (2H, m).

### Example 8

A mixture of 3-{3-[4-(trifluoromethyl)phenyl]-5-isoxazolyl}-1-propyl methanesulfonate (1.04 g), sodium iodide (500 mg), methyl 3-hydroxy-1-phenyl-1H-pyrazole-5-carboxylate (650 mg), potassium carbonate (500 mg) and N,N-dimethylformamide (10 ml) was stirred at 90° C. for 5 hours. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours. 1N Hydrochloric acid (5 ml) was added, and extracted with ethyl acetate. The ethyl acetate layer was washed with saturated aqueous sodium chloride solution, dried (MgSO₄) and concentrated. The obtained colorless crystals were collected by filtration to give 1-phenyl-3-(3-{3-[4-(trifluoromethyl)phenyl]-5-isoxazolyl}propoxy)-1H-pyrazole-5-carboxylic acid (1.16 g, yield 85%). The crystals were recrystallized from ethyl acetate-hexane. melting point: 145-146° C.

¹H-NMR (CDCl₃)δ: 2.16-2.36 (2H, m), 2.96-3.10 (2H, m), 4.24-4.36 (2H, m), 6.40 (1H, s), 6.50 (1H, s), 7.36-7.47 (5H, m), 7.65-7.75 (2H, m), 7.84-7.94 (2H, m).

### Example 9

To a mixture of {3-methyl-1-[5-(trifluoromethyl)-2-pyridyl]-1H-pyrazol-4-yl}methanol (500 mg), methyl 3-(4-hydroxyphenyl)propionate (370 mg), triphenylphosphine (530 mg) and tetrahydrofuran (10 ml) was dropwise added a 40% solution of diethyl azodicarboxylate in toluene (900 mg) at room temperature, and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (3 ml), tetrahydrofuran (5 ml) and methanol (5 ml) was stirred at room temperature for 5 hours. 1N Hydrochloric acid (3 ml) was added and extracted with ethyl acetate. The ethyl acetate layer was washed with saturated aqueous sodium chloride solution, dried (MgSO₄) and concentrated. The obtained colorless crystals were collected by filtration to give 3-(4-{3-methyl-1-[5-(trifluoromethyl)-2-pyridyl]-1H-pyrazol-4-ylmethoxy}phenyl)propionic acid (620 mg, yield 79%). The crystals were recrystallized from ethyl acetate-hexane. melting point: 195-196° C.

¹H-NMR (CDCl₃)δ: 2.39 (3H, s), 4.64 (2H, s), 4.94 (2H, s), 6.87-6.97 (4H, m), 7.96-8.06 (2H, m), 8.55 (1H, s), 8.61-8.66 (1H, m).

### Example 10

To a mixture of {3-methyl-1-[5-(trifluoromethyl)-2-pyridyl]-1H-pyrazol-4-yl}methanol (900 mg), methyl (4-hydroxyphenoxy)acetate (650 mg), triphenylphosphine (930 mg) and tetrahydrofuran (10 ml) was dropwise added a 40% solution (1.59 g) of diethyl azodicarboxylate in toluene at room temperature, and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours. 1N Hydrochloric acid (5 ml) was added, and the mixture was extracted with ethyl acetate. The ethyl acetate layer was washed with saturated aqueous sodium chloride solution, dried (MgSO₄) and concentrated. The obtained colorless crystals were collected by filtration to give (4-{3-methyl-1-[5-(trifluoromethyl)-2-pyridyl]-1H-pyrazol-4-ylmethoxy}phenoxy)acetic acid (610 mg, yield 43%). The crystals were recrystallized from ethyl acetate-hexane. melting point: 138-139° C.

¹H-NMR (CDCl₃)δ: 2.39 (3H, s), 4.64 (2H, s), 4.94 (2H, s), 6.87-6.97 (4H, m), 7.96-8.06 (2H, m), 8.55 (1H, s), 8.61-8.66 (1H, m).

### Example 11

To a mixture of 4-{3-[4-(trifluoromethyl)phenyl]-5-isoxazolyl}-1-butanol (740 mg), ethyl 3-(3-hydroxy-1-phenyl-1H-pyrazol-5-yl)propionate (670 mg), triphenylphosphine (700 mg) and tetrahydrofuran (10 ml) was dropwise added a 40% solution (1.20 g) of diethyl azodicarboxylate in toluene at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and ethanol (5 ml) was stirred at room temperature for 5 hours. 1N Hydrochloric acid (5 ml) was added and the mixture was extracted with ethyl acetate. The ethyl acetate layer was washed with saturated aqueous sodium chloride solution, dried (MgSO₄) and concentrated. The obtained colorless crystals were collected by filtration to give 3-[1-phenyl-3-(4-{3-[4-(trifluoromethyl)phenyl]-5-isoxazolyl}butoxy)-1H-pyrazol-5-yl]propionic acid (930 mg, yield 72%). The crystals were recrystallized from ethyl acetate-hexane. melting point: 139-140° C.

¹H-NMR (CDCl₃)δ: 1.76-2.06 (4H, m), 2.56-2.70 (2H, m), 2.84-3.02 (4H, m), 4.18-4.32 (2H, m), 5.68 (1H, s), 6.36 (1H, s) 7.28-7.48 (5H, m), 7.66-7.75 (2H, m), 7.85-7.94 (2H, m).

### Example 12

A mixture of 4-{3-[4-(trifluoromethyl)phenyl]-5-isoxazolyl}-1-butyl methanesulfonate (700 mg), sodium iodide (300 mg), methyl 4-hydroxybenzoate (290 mg), potassium carbonate (460 mg) and N,N-dimethylformamide (10 ml) was stirred at 90° C. for 5 hours. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (3 ml), tetrahydrofuran (5 ml) and methanol (5 ml) was stirred at room temperature for 5 hours. 1N Hydrochloric acid (3 ml) was added, and the mixture was extracted with ethyl acetate. The ethyl acetate layer was washed with saturated aqueous sodium chloride solution, dried (MgSO₄) and concentrated. The obtained colorless crystals were collected by filtration to give 4-(4-{3-[4-(trifluoromethyl)phenyl]-5-isoxazolyl}butoxy)benzoic acid (630 mg, yield 81%). The crystals were recrystallized from ethyl acetate-hexane. melting point: 170-171° C.

H-NMR (CDCl₃)δ: 1.82-2.12 (4H, m), 2.86-2.98 (2H, m), 4.02-4.14 (2H, m), 6.36 (1H, s), 6.88-6.98 (2H, m), 7.66-7.76 (2H, m), 7.85-7.95 (2H, m), 8.00-8.10 (2H, m).

### Example 13

To a mixture of 4-{3-[4-(trifluoromethyl)phenyl]-5-isoxazolyl}-1-butanol (700 mg), methyl 4-hydroxyphenylacetate (400 mg), triphenylphosphine (660 mg) and tetrahydrofuran (10 ml) was dropwise added a 40% solution (1.10 g) of diethyl azodicarboxylate in toluene at room temperature and the mixture was stirred overnight. The reaction solution was concentrated. The residue was subjected to silica gel column chromatography, and a colorless oil was obtained from a fraction eluted with ethyl acetate-hexane (1:4, volume ratio).

A mixture of the obtained oily substance, 1N aqueous sodium hydroxide solution (5 ml), tetrahydrofuran (5 ml) and methanol (5 ml) was stirred at room temperature for 5 hours. 1N Hydrochloric acid (5 ml) was added and the mixture was extracted with ethyl acetate. The ethyl acetate layer was washed with saturated aqueous sodium chloride solution, dried (MgSO₄) and concentrated. The obtained colorless crystals were collected by filtration to give [4-(4-{3-[4-(trifluoromethyl)phenyl]-5-isoxazolyl}butoxy)phenyl]acetic acid (810 mg, yield 80%). The crystals were recrystallized from ethyl acetate-hexane. melting point: 125-126° C.

¹H-NMR (CDCl₃)δ: 1.78-2.07 (4H, m), 2.83-2.95 (2H, m), 3.59 (2H, s), 3.94-4.06 (2H, m), 6.36 (1H, s), 6.79-6.91 (2H, m), 7.14-7.26 (2H, m), 7.64-7.76 (2H, m), 7.84-7.96 (2H, m).

### Example 14

To a mixture of 4-{3-[4-(trifluoromethyl)phenyl]-5-isoxazolyl}-1-butanol (700 mg), methyl 3-(4-hydroxyphenyl)propionate (440 mg), triphenylphosphine (650 mg) and tetrahydrofuran (10 ml) was dropwise added a 40% solution (1.25 g) of diethyl azodicarboxylate in toluene at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours. 1N Hydrochloric acid (5 ml) was added and the mixture was extracted with ethyl acetate. The ethyl acetate layer was washed with saturated aqueous sodium chloride solution, dried (MgSO₄) and concentrated. The obtained colorless crystals were collected by filtration to give 3-[4-(4-{3-[4-(trifluoromethyl)phenyl]-5-isoxazolyl}butoxy)phenyl]propionic acid (760 mg, yield 72%). The crystals were recrystallized from ethyl acetate-hexane. melting point: 130-131° C.

¹H-NMR (CDCl₃)δ: 1.80-2.04 (4H, m), 2.56-2.70 (2H, m), 2.82-2.98 (4H, m), 3.94-4.06 (2H, m), 6.36 (1H, s), 6.77-6.88 (2H, m), 7.07-7.17 (2H, m), 7.64-7.76 (2H, m), 7.85-7.96 (2H, m).

### Example 15

To a mixture of 4-{3-[4-(trifluoromethyl)phenyl]-5-isoxazolyl}-1-butanol (700 mg), methyl 2-(4-hydroxyphenoxy)-2-methylpropionate (500 mg), triphenylphosphine (650 mg) and tetrahydrofuran (10 ml) was dropwise added a 40% solution (1.10 g) of diethyl azodicarboxylate in toluene at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours. 1N Hydrochloric acid (5 ml) was added and the mixture was extracted with ethyl acetate. The ethyl acetate layer was washed with saturated aqueous sodium chloride solution, dried (MgSO₄) and concentrated. The obtained colorless crystals were collected by filtration to give 2-methyl-2-[4-(4-{3-[4-(trifluoromethyl)phenyl]-5-isoxazolyl}butoxy)phenoxy]propionic acid (860 mg, yield 78%). The crystals were recrystallized from ethyl acetate-hexane. melting point: 103-104° C.

¹H-NMR (CDCl₃)δ: 1.53 (6H, s), 1.80-2.06 (4H, m), 2.86-2.98 (2H, m), 3.94-4.04 (2H, m), 6.36 (1H, s), 6.72-6.95 (4H, m), 7.66-7.75 (2H, m), 7.85-7.94 (2H, m).

### Example 16

To a mixture of 4-{3-[4-(trifluoromethyl)phenyl]-5-isoxazolyl}-1-butanol (700 mg), methyl 3-hydroxyphenylacetate (420 mg), triphenylphosphine (650 mg) and tetrahydrofuran (10 ml) was dropwise added a 40% solution (1.13 g) of diethyl azodicarboxylate in toluene at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours. 1N Hydrochloric acid (5 ml) was added and the mixture was extracted with ethyl acetate. The ethyl acetate layer was washed with saturated aqueous sodium chloride solution, dried (MgSO₄) and concentrated. The obtained colorless crystals were collected by filtration to give [3-(4-{3-[4-(trifluoromethyl)phenyl]-5-isoxazolyl}butoxy)phenyl]acetic acid (800 mg, yield 78%). The crystals were recrystallized from ethyl acetate-hexane. melting point: 134-135° C.

¹H-NMR (CDCl₃)δ: 1.80-2.08 (4H, m), 2.84-2.96 (2H, m), 3.62 (2H, s), 3.96-4.06 (2H, m), 6.36 (1H, s), 6.76-6.91 (3H, m), 7.18-7.30 (1H, m), 7.64-7.76 (2H, m), 7.85-7.96 (2H, m).

### Example 17

To a mixture of 4-{3-[4-(trifluoromethyl)phenyl]-5-isoxazolyl}-1-butanol (700 mg), methyl 2-hydroxyphenylacetate (420 mg), triphenylphosphine (650 mg) and tetrahydrofuran (10 ml) was dropwise added a 40% solution (1.10 g) of diethyl azodicarboxylate in toluene at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours. 1N Hydrochloric acid (5 ml) was added and the mixture was extracted with ethyl acetate. The ethyl acetate layer was washed with saturated aqueous sodium chloride solution, dried (MgSO₄) and concentrated. The obtained colorless crystals were collected by filtration to give [2-(4-{3-[4-(trifluoromethyl)phenyl]-5-isoxazolyl}butoxy)phenyl]acetic acid (800 mg, yield 78%). The crystals were recrystallized from ethyl acetate-hexane. melting point: 122-123° C.

¹H-NMR (CDCl₃)δ: 1.78-2.06 (4H, m), 2.78-2.92 (2H, m), 3.65 (2H, s), 3.96-4.07 (2H, m), 6.36 (1H, s), 6.80-6.96 (2H, m), 7.14-7.30 (2H, m), 7.64-7.74 (2H, m), 7.84-7.94 (2H, m).

### Example 18

To a mixture of 3-{3-ethoxy-1-[5-(trifluoromethyl)-2-pyridyl]-1H-pyrazol-4-yl}-1-propanol (330 mg), methyl 2-(4-hydroxyphenoxy)-2-methylpropionate (250 mg), triphenylphosphine (310 mg) and tetrahydrofuran (7 ml) was dropwise added a 40% solution (550 mg) of diethyl azodicarboxylate in toluene at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours. 1N Hydrochloric acid (5 ml) was added and the mixture was extracted with ethyl acetate. The ethyl acetate layer was washed with saturated aqueous sodium chloride solution, dried (MgSO₄) and concentrated. The obtained colorless crystals were collected by filtration to give 2-[4-(3-{3-ethoxy-1-[5-(trifluoromethyl)-2-pyridyl]-1H-pyrazol-4-yl}propoxy)phenoxy]-2-methylpropionic acid (370 mg, yield 71%). The crystals were recrystallized from ethyl acetate-hexane. melting point: 91-92° C.

¹H-NMR (CDCl₃)δ: 1.41 (3H, t, J=7.0 Hz), 1.54 (6H, s), 2.00-2.18 (2H, m), 2.54-2.66 (2H, m), 3.98 (2H, t, J=6.2 Hz), 4.35 (2H, q, J=7.0 Hz), 6.76-6.96 (4H, m), 7.81 (1H, d, J=8.8 Hz), 7.91 (1H, dd, J=2.0, 8.8 Hz), 8.18 (1H, s), 8.55 (1H, d, J=2.0 Hz).

### Example 19

To a mixture of {3-methyl-1-[5-(trifluoromethyl)-2-pyridyl]-1H-pyrazol-4-yl}methanol (250 mg), ethyl 3-(2-ethoxy-4-hydroxyphenyl)propionate (250 mg), triphenylphosphine (280 mg) and tetrahydrofuran (10 ml) was dropwise added a 40% solution (480 mg) of diethyl azodicarboxylate in toluene at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and ethanol (5 ml) was stirred at room temperature for 5 hours. 1N Hydrochloric acid (5 ml) was added and the mixture was extracted with ethyl acetate. The ethyl acetate layer was washed with saturated aqueous sodium chloride solution, dried (MgSO₄) and concentrated. The obtained colorless crystals were collected by filtration to give 3-(2-ethoxy-4-{3-methyl-1-[5-(trifluoromethyl)-2-pyridyl]-1H-pyrazol-4-ylmethoxy}phenyl)propionic acid (310 mg, yield 71%). The crystals were recrystallized from ethyl acetate-hexane. melting point: 151-152° C.

¹H-NMR (CDCl₃)δ: 1.42 (3H, t, J=7.0 Hz), 2.39 (3H, s), 2.60-2.71 (2H, m), 2.84-2.95 (2H, m), 4.01 (2H, q, J=7.0 Hz), 4.94 (2H, s), 6.45-6.54 (2H, m), 7.06-7.14 (1H, m), 7.94-8.08 (2H, m), 8.56 (1H, s), 8.61-8.68 (1H, m).

### Example 20

To a mixture of 4-{3-[4-(trifluoromethyl)phenyl]-5-isoxazolyl}-1-butanol (1.10 g), methyl 3-(3-hydroxyphenyl)propionate (780 mg), triphenylphosphine (1.10 g) and tetrahydrofuran (15 ml) was dropwise added a 40% solution (1.75 g) of diethyl azodicarboxylate in toluene at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (7 ml), tetrahydrofuran (7 ml) and methanol (7 ml) was stirred at room temperature for 5 hours. 1N Hydrochloric acid (7 ml) was added, and the mixture was extracted with ethyl acetate. The ethyl acetate layer was washed with saturated aqueous sodium chloride solution, dried (MgSO₄) and concentrated. The obtained colorless crystals were collected by filtration to give 3-[(3-(4-{3-[4-(trifluoromethyl)phenyl]-5-isoxazolyl}butoxy)phenyl]propionic acid (1.26 g, yield 75%). The crystals were recrystallized from ethyl acetate-hexane. melting point: 131-132° C.

¹H-NMR (CDCl₃)δ: 1.80-2.08 (4H, m), 2.60-2.74 (2H, m), 2.85-3.00 (4H, m), 3.96-4.06 (2H, m), 6.36 (1H, s), 6.72-6.84 (3H, m), 7.15-7.27 (1H, m), 7.67-7.76 (2H, m), 7.86-7.95 (2H, m).

### Example 21

To a mixture of 4-{3-[4-(trifluoromethyl)phenyl]-5-isoxazolyl}-1-butanol (570 mg), ethyl 3-(2-ethoxy-4-hydroxyphenyl)propionate (480 mg), triphenylphosphine (550 mg) and tetrahydrofuran (10 ml) was dropwise added a 40% solution (950 mg) of diethyl azodicarboxylate in toluene at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and ethanol (5 ml) was stirred at room temperature for 5 hours. 1N Hydrochloric acid (5 ml) was added and the mixture was extracted with ethyl acetate. The ethyl acetate layer was washed with saturated aqueous sodium chloride solution, dried (MgSO₄) and concentrated. The obtained colorless crystals were collected by filtration to give 3-[2-ethoxy-4-(4-{3-[4-(trifluoromethyl)phenyl]-5-isoxazolyl}butoxy)phenyl]propionic acid (260 mg, yield 27%). The crystals were recrystallized from ethyl acetate-hexane. melting point: 105-106° C.

¹H-NMR (CDCl₃)δ: 1.41 (3H, t, J=7.0 Hz), 1.78-2.08 (4H, m), 2.54-2.72 (2H, m), 2.82-2.97 (4H, m), 3.92-4.08 (4H, m), 6.32-6.44 (3H, m), 6.98-7.10 (1H, m), 7.66-7.76 (2H, m), 7.85-7.95 (2H, m).

### Example 22

To a mixture of 3-{3-isopropyl-1-[5-(trifluoromethyl)-2-pyridyl]-1H-pyrazol-4-yl}-1-propanol (410 mg), methyl 3-hydroxyphenylacetate (230 mg), triphenylphosphine (370 mg) and tetrahydrofuran (10 ml) was dropwise added a 40% solution (630 mg) of diethyl azodicarboxylate in toluene at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours. 1N Hydrochloric acid (5 ml) was added and the mixture was extracted with ethyl acetate. The ethyl acetate layer was washed with saturated aqueous sodium chloride solution, dried (MgSO₄) and concentrated. The obtained colorless crystals were collected by filtration to give [3-(3-{3-isopropyl-1-[5-(trifluoromethyl)-2-pyridyl]-1H-pyrazol-4-yl}propoxy)phenyl]acetic acid (330 mg, yield 56%). The crystals were recrystallized from ethyl acetate-hexane. melting point: 82-83° C.

¹H-NMR (CDCl₃)δ: 1.47 (6H, d, J=7.0 Hz), 2.02-2.21 (2H, m), 2.69 (2H, t, J=7.4 Hz), 2.94-3.12 (1H, m), 3.64 (2H, s), 4.05 (2H, t, J=6.0 Hz), 6.80-6.92 (3H, m), 7.19-7.30 (1H, m), 7.95 (1H, dd, J=1.8, 8.4 Hz), 8.05 (1H, d, J=8.4 Hz), 8.29 (1H, s), 8.57-8.64 (1H, m).

### Example 23

To a mixture of 3-(3-isopropyl-1-[5-(trifluoromethyl)-2-pyridyl]-1H-pyrazol-4-yl)-1-propanol (380 mg), methyl 3-(3-hydroxyphenyl)propionate (220 mg), tributylphosphine (260 mg) and tetrahydrofuran (10 ml) was added 1,1′-azodicarbonyldipiperidine (350 mg) at room temperature, and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours. 1N Hydrochloric acid (5 ml) was added and the mixture was extracted with ethyl acetate. The ethyl acetate layer was washed with saturated aqueous sodium chloride solution, dried (MgSO₄) and concentrated. The obtained colorless crystals were collected by filtration to give 3-[3-(3-{3-isopropyl-1-[5-(trifluoromethyl)-2-pyridyl]-1H-pyrazol-4-yl}propoxy)phenyl]propionic acid (380 mg, yield 68%). The crystals were recrystallized from ethyl acetate-hexane. melting point: 102-103° C.

¹H-NMR (CDCl₃)δ: 1.32 (6H, d, J=7.0 Hz), 2.00-2.20 (2H, m), 2.62-2.76 (4H, m), 2.87-3.13 (3H, m), 4.05 (2H, t, J=6.2 Hz), 6.73-6.86 (3H, m), 7.15-7.26 (1H, m), 7.91-8.08 (2H, m), 8.27 (1H, s), 8.57-8.63 (1H, m).

### Example 24

To a mixture of 3-{3-isopropyl-1-[5-(trifluoromethyl)-2-pyridyl]-1H-pyrazol-4-yl}-1-propanol (520 mg), ethyl 3-(3-hydroxy-1-phenyl-1H-pyrazol-5-yl)propionate (440 mg), tributylphosphine (510 mg) and tetrahydrofuran (10 ml) was added 1,1′-azodicarbonyldipiperidine (650 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours. 1N Hydrochloric acid (5 ml) was added and the mixture was extracted with ethyl acetate. The ethyl acetate layer was washed with saturated aqueous sodium chloride solution, dried (MgSO₄) and concentrated. The obtained colorless crystals were collected by filtration to give 3-[3-(3-{3-isopropyl-1-[5-(trifluoromethyl)-2-pyridyl]-1H-pyrazol-4-yl}propoxy)-1-phenyl-1H-pyrazol-5-yl]propionic acid (420 mg, yield 48%). The crystals were recrystallized from ethyl acetate-hexane. melting point: 139-140° C.

¹H-NMR (CDCl₃)δ: 1.32 (6H, d, J=7.0 Hz), 2.00-2.20 (2H, m), 2.56-2.76 (4H, m), 2.88-3.12 (3H, m), 4.27 (2H, t, J=6.0 Hz), 5.72 (1H, s), 7.30-7.50 (5H, m), 7.95 (1H, dd, J=2.6, 9.0 Hz), 8.04 (1H, d, J=9.0 Hz), 8.27 (1H, s), 8.54-8.61 (1H, m).

### Example 25

To a mixture of 3-{3-isopropyl-1-[5-(trifluoromethyl)-2-pyridyl]-1H-pyrazol-4-yl}-1-propanol (550 mg), ethyl 3-(3-hydroxy-1-methyl-1H-pyrazol-5-yl)propionate (360 mg), tributylphosphine (530 mg) and tetrahydrofuran (10 ml) was added 1,1′-azodicarbonyldipiperidine (670 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and ethanol (5 ml) was stirred at room temperature for 5 hours. 1N Hydrochloric acid (5 ml) was added and the mixture was extracted with ethyl acetate. The ethyl acetate layer was washed with saturated aqueous sodium chloride solution, dried (MgSO₄) and concentrated. The obtained colorless crystals were collected by filtration to give 3-[3-(3-{3-isopropyl-1-[5-(trifluoromethyl)-2-pyridyl]-1H-pyrazol-4-yl}propoxy)-1-methyl-1H-pyrazol-5-yl]propionic acid (630 mg, yield 77%). The crystals were recrystallized from ethyl acetate-hexane. melting point: 131-132° C.

¹H-NMR (CDCl₃)δ:1.31 (6H, d, J=7.0 Hz), 1.98-2.16 (2H, m), 2.58-3.12 (7H, m), 3.66 (3H, s), 4.16 (2H, t, J=6.2 Hz), 5.49 (1H s), 7.94 (1H, dd, J=1.8, 8.6 Hz), 8.04 (1H, d, J=8.6 Hz), 8.26 (1H, s), 8.56-8.62 (1H, m).

### Example 26

To a mixture of 3-{3-isopropyl-1-[5-(trifluoromethyl)-2-pyridyl]-1H-pyrazol-4-ylmethoxy}-1-methyl-1H-pyrazole-5-carbaldehyde (1.10 g), ethyl diethylphosphonoacetate (690 mg) and N,N-dimethylformamide (15 ml), was added sodium hydride (60%, in oil, 120 mg) at 0° C., and the mixture was stirred overnight at room temperature. The reaction mixture was poured into water, and extracted with ethyl acetate. The ethyl acetate layer was washed with dilute hydrochloric acid and then with saturated aqueous sodium chloride solution, dried (MgSO₄) and concentrated. The residue was subjected to silica gel column chromatography, and ethyl (E)-3-(3-{3-isopropyl-1-[5-(trifluoromethyl)-2-pyridyl]-1H-pyrazol-4-ylmethoxy}-1-methyl-1H-pyrazol-5-yl)propenoate (1.03 g, yield 79%) was obtained as colorless crystals from a fraction eluted with ethyl acetate-hexane (1:4, volume ratio). The crystals were recrystallized from ethyl acetate-hexane. melting point: 105-106° C.

¹H-NMR (CDCl₃)δ: 1.33 (3H, t, J=7.0 Hz), 1.36 (6H, d, J=7.0 Hz), 3.07-3.24 (1H, m), 3.83 (3H, s), 4.27 (2H, q, J=7.0 Hz), 5.14 (2H, s), 5.95 (1H, s), 6.28 (1H, d, J=15.6 Hz), 7.48 (1H, d, J=15.6 Hz), 7.97 (1H, dd, J=2.2, 8.4 Hz), 8.07 (1H, d, J=8.4 Hz), 8.56 (1H, s), 8.60-8.66 (1H, m).

### Example 27

A mixture of ethyl (E)-3-(3-{3-isopropyl-1-[5-(trifluoromethyl)-2-pyridyl]-1H-pyrazol-4-ylmethoxy}-1-methyl-1H-pyrazol-5-yl)propenoate (900 mg), 5% palladium-carbon (260 mg) and tetrahydrofuran (20 ml) was stirred at room temperature for 1 hour under a hydrogen atmosphere. Palladium-carbon was removed by filtration and the filtrate was concentrated. A mixture of the obtained crystal, 1N aqueous sodium hydroxide solution (5 ml), tetrahydrofuran (5 ml) and methanol (5 ml) was stirred at room temperature for 3 hours. 1N Hydrochloric acid (5 ml) was added and the mixture was extracted with ethyl acetate. The ethyl acetate layer was washed with saturated aqueous sodium chloride solution, dried (MgSO₄) and concentrated. The obtained colorless crystals were collected by filtration to give 3-(3-{3-isopropyl-1-[5-(trifluoromethyl)-2-pyridyl]-1H-pyrazol-4-ylmethoxy}-1-methyl-1H-pyrazol-5-yl)propionic acid (780 mg, yield 92%). The crystals were recrystallized from ethyl acetate-hexane. melting point: 141-142° C.

¹H-NMR (CDCl₃)δ: 1.36 (6H, d, J=7.0 Hz), 2.62-2.94 (4H, m), 3.06-3.24 (1H, m), 3.69 (3H, s), 5.10 (2H, s), 5.51 (1H, s), 7.98 (1H, dd, J=2.2, 9.2 Hz), 8.07 (1H, d, J=9.2 Hz), 8.53 (1H, s), 8.58-8.67 (1H, m).

### Example 28

To a mixture of 3-{3-isopropyl-1-[5-(trifluoromethyl)-2-pyridyl]-1H-pyrazol-4-yl}-1-propanol (1.20 g), methyl 2-(3-hydroxyphenoxy)-2-methylpropionate (830 mg), tributylphosphine (1.60 g) and tetrahydrofuran (20 ml) was added 1,1′-azodicarbonyldipiperidine (2.01 g) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours. 1N Hydrochloric acid (5 ml) was added and the mixture was extracted with ethyl acetate. The ethyl acetate layer was washed with saturated aqueous sodium chloride solution, dried (MgSO₄) and concentrated. The obtained colorless crystals were collected by filtration to give 2-[3-{3-(3-isopropyl-1-[5-(trifluoromethyl)-2-pyridyl]-1H-pyrazol-4-yl}propoxy)phenoxy]-2-methylpropionic acid (1.32 g, yield 70%). The crystals were recrystallized from ethyl acetate-hexane. melting point: 101-102° C.

¹H-NMR (CDCl₃)δ: 1.32 (6H, d, J=7.0 Hz), 1.63 (6H, s), 2.00-2.18 (2H, m), 2.69 (2H, t, J=7.2 Hz), 2.94-3.12 (1H, m), 4.00 (2H, t, J=6.2 Hz), 6.50-6.70 (3H, m), 7.11-7.24 (1H, m), 7.96 (1H, dd, J=2.2, 8.8 Hz), 8.06 (1H, d, J=8.8 Hz), 8.26 (1H, s), 8.54-8.63 (1H, m).

### Example 29

To a mixture of 3-{3-ethoxy-1-[5-(trifluoromethyl)-2-pyridyl]-1H-pyrazol-4-yl}-1-propanol (550 mg), methyl 2-(3-hydroxyphenoxy)-2-methylpropionate (380 mg), tributylphosphine (730 mg) and tetrahydrofuran (10 ml) was added 1,1′-azodicarbonyldipiperidine (910 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours. 1N Hydrochloric acid (5 ml) was added and the mixture was extracted with ethyl acetate. The ethyl acetate layer was washed with saturated aqueous sodium chloride solution, dried (MgSO₄) and concentrated. The obtained colorless crystals were collected by filtration to give 2-[3-(3-{3-ethoxy-1-[5-(trifluoromethyl)-2-pyridyl]-1H-pyrazol-4-yl}propoxy)phenoxy]-2-methylpropionic acid (530 mg, yield 62%). The crystals were recrystallized from ethyl acetate-hexane. melting point: 120-121° C.

¹H-NMR (CDCl₃)δ: 1.41 (3H, t, J=7.0 Hz), 1.62 (6H, s), 1.96-2.18 (2H, m), 2.62 (2H, t, J=7.0 Hz), 3.97 (2H, t, J=6.2 Hz), 4.35 (2H, q, J=7.0 Hz), 6.48-6.68 (3H, m), 7.08-7.23 (1H, m), 7.84 (1H, d, J=8.8 Hz), 7.93 (1H, dd, J=2.6, 8.8 Hz), 8.16 (1H, s), 8.51-8.56 (1H, m).

### Example 30

To a mixture of 3-{3-ethoxy-1-[5-(trifluoromethyl)-2-pyridyl]-1H-pyrazol-4-yl}-1-propanol (650 mg), methyl 3-hydroxyphenylacetate (380 mg), tributylphosphine (930 mg) and tetrahydrofuran (10 ml) was added 1,1′-azodicarbonyldipiperidine (1.16 g) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours. 1N Hydrochloric acid (5 ml) was added and the mixture was extracted with ethyl acetate. The ethyl acetate layer was washed with saturated aqueous sodium chloride solution, dried (MgSO₄) and concentrated. The obtained colorless crystals were collected by filtration to give [3-(3-{3-ethoxy-1-[5-(trifluoromethyl)-2-pyridyl]-1H-pyrazol-4-yl}propoxy)phenyl]acetic acid (490 mg, yield 53%). The crystals were recrystallized from ethyl acetate-hexane. melting point: 134-135° C.

¹H-NMR (CDCl₃)δ: 1.41 (3H, t, J=7.2 Hz), 2.02-2.14 (2H, m), 2.60 (2H, t, J=7.2 Hz), 3.62 (2H, s), 4.01 (2H, t, J=6.3 Hz), 4.34 (2H, q, J=7.2 Hz), 6.78-6.88 (3H, m), 7.18-7.28 (1H, m), 7.80 (1H, d, J=8.7 Hz), 7.90 (1H, dd, J=2.4, 8.7 Hz), 8.17 (1H, s), 8.52-8.57 (1H, m).

### Example 31

To a mixture of 3-{3-ethoxy-1-[5-(trifluoromethyl)-2-pyridyl]-1H-pyrazol-4-yl}-1-propanol (620 mg), methyl 2-hydroxyphenylacetate (340 mg), tributylphosphine (800 mg) and tetrahydrofuran (10 ml) was added 1,1′-azodicarbonyldipiperidine (1.00 g) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours. 1N Hydrochloric acid (5 ml) was added and the mixture was extracted with ethyl acetate. The ethyl acetate layer was washed with saturated aqueous sodium chloride solution, dried (MgSO₄) and concentrated. The obtained colorless crystals were collected by filtration to give (2-(3-{3-ethoxy-1-[5-(trifluoromethyl)-2-pyridyl]-1H-pyrazol-4-yl}propoxy)phenyl]acetic acid (310 mg, yield 35%). The crystals were recrystallized from ethyl acetate-hexane. melting point: 83-84° C.

¹H-NMR (CDCl₃)δ: 1.40 (3H, t, J=7.0 Hz), 2.00-2.18 (2H, m), 2.61 (2H, t, J=7.0 Hz), 3.68 (2H, s), 4.02 (2H, t, J=6.2 Hz), 4.34 (2H, q, J=7.0 Hz), 6.80-6.96 (2H, m), 7.14-7.28 (2H, m), 7.80 (1H, d, J=8.8 Hz), 7.90 (1H, dd, J=2.2, 8.8 Hz), 8.20 (1H, s), 8.49-8.56 (1H, m).

### Example 32

To a mixture of {3-methyl-1-[5-(trifluoromethyl)-2-pyridyl]-1H-pyrazol-4-yl}methanol (500 mg), methyl 2-(3-hydroxyphenoxy)-2-methylpropionate (430 mg), triphenylphosphine (570 mg) and tetrahydrofuran (10 ml) was dropwise added a 40% solution (980 mg) of diethyl azodicarboxylate in toluene at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and ethanol (5 ml) was stirred at room temperature for 5 hours. 1N Hydrochloric acid (5 ml) was added and the mixture was extracted with ethyl acetate. The ethyl acetate layer was washed with saturated aqueous sodium chloride solution, dried (MgSO₄) and concentrated. The obtained colorless crystals were collected by filtration to give 2-methyl-2-(3-{3-methyl-1-[5-(trifluoromethyl)-2-pyridyl]-1H-pyrazol-4-ylmethoxy}phenoxy)propionic acid (600 mg, yield 71%). The crystals were recrystallized from ethyl acetate-hexane. melting point: 152-153° C.

¹H-NMR (CDCl₃)δ: 1.64 (6H, s), 2.38 (3H, s), 4.99 (2H, s), 6.52-6.68 (3H, m), 7.15 (1H, t, J=8.1 Hz), 7.98-8.08 (2H, m), 8.58-8.68 (2H, m).

### Example 33

To a mixture of 3-{3-ethoxy-1-[5-(trifluoromethyl)-2-pyridyl]-1H-pyrazol-4-yl}-1-propanol (550 mg), methyl 3-(3-hydroxyphenyl)propionate (330 mg), tributylphosphine (700 mg) and tetrahydrofuran (10 ml) was added 1,1′-azodicarbonyldipiperidine (880 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours. 1N Hydrochloric acid (5 ml) was added and the mixture was extracted with ethyl acetate. The ethyl acetate layer was washed with saturated aqueous sodium chloride solution, dried (MgSO₄) and concentrated. The obtained colorless crystals were collected by filtration to give 3-[3-(3-{3-ethoxy-1-[5-(trifluoromethyl)-2-pyridyl]-1H-pyrazol-4-yl}propoxy)phenyl]propionic acid (590 mg, yield 73%). The crystals were recrystallized from isopropyl ether-hexane. melting point: 88-89° C.

¹H-NMR (CDCl₃)δ: 1.41 (3H, t, J=7.0 Hz), 2.00-2.18 (2H, m), 2.54-2.76 (4H, m), 2.88-3.02 (2H, m), 4.00 (2H, t, J=6.2 Hz), 4.35 (2H, q, J=7.0 Hz), 6.71-6.88 (3H, m), 7.14-7.24 (1H, m), 7.77-7.96 (2H, m), 8.17 (1H, s), 8.52-8.60 (1H, m).

### Example 34

To a mixture of {3-methyl-1-[5-(trifluoromethyl)-2-pyridyl]-1H-pyrazol-4-yl}methanol (520 mg), methyl 2-(4-hydroxyphenoxy)-2-methylpropionate (430 mg), triphenylphosphine (580 mg) and tetrahydrofuran (10 ml) was dropwise added a 40% solution (980 mg) of diethyl azodicarboxylate in toluene at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and ethanol (5 ml) was stirred at room temperature for 5 hours. 1N Hydrochloric acid (5 ml) was added and the mixture was extracted with ethyl acetate. The ethyl acetate layer was washed with saturated aqueous sodium chloride solution, dried (MgSO₄) and concentrated. The obtained colorless crystals were collected by filtration to give 2-methyl-2-(4-{3-methyl-1-[5-(trifluoromethyl)-2-pyridyl]-1H-pyrazol-4-ylmethoxy}phenoxy)propionic acid (330 mg, yield 38%). The crystals were recrystallized from isopropyl ether-hexane. melting point: 106-107° C.

¹H-NMR (CDCl₃)δ: 1.55 (6H, s), 2.39 (3H, s), 4.94 (2H, s), 6.85-6.99 (4H, m), 7.95-8.07 (2H, m), 8.55 (1H, s), 8.61-8.66 (1H, m).

### Example 35

To a mixture of 3-{3-ethoxy-1-[5-(trifluoromethyl)-2-pyridyl]-1H-pyrazol-4-yl}-1-propanol (500 mg), ethyl 3-(2-ethoxy-4-hydroxyphenyl)propionate (460 mg), tributylphosphine (650 mg) and tetrahydrofuran (10 ml) was added 1,1′-azodicarbonyldipiperidine (820 mg) at room temperature and the ixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours. 1N Hydrochloric acid (5 ml) was added and the mixture was extracted with ethyl acetate. The ethyl acetate layer was washed with saturated aqueous sodium chloride solution, dried (MgSO₄) and concentrated. The obtained colorless crystals were collected by filtration to give 3-[2-ethoxy-4-(3-{3-ethoxy-1-[5-(trifluoromethyl)-2-pyridyl]-1H-pyrazol-4-yl}propoxy)phenyl]propionic acid (540 mg, yield 67%). The crystals were recrystallized from ethyl acetate-hexane. melting point: 96-97° C.

¹H-NMR (CDCl₃)δ: 1.37-1.48 (6H, m), 2.02-2.16 (2H, m), 2.56-2.69 (4H, m), 2.83-2.94 (2H, m), 3.93-4.06 (4H, m), 4.34 (2H, q, J=7.2 Hz), 6.34-6.47 (2H, m), 7.02 (1H, d, J=8.4 Hz), 7.76-7.94 (2H, m), 8.17 (1H, s), 8.50-8.58 (1H, m).

### Example 36

To a mixture of 1-methyl-3-{3-methyl-1-[5-(trifluoromethyl)-2-pyridyl]-1H-pyrazol-4-ylmethoxy}-1H-pyrazole-5-carbaldehyde (2.00 g), ethyl diethylphosphonoacetate (1.35 g) and N,N-dimethylformamide (30 ml) was added sodium hydride (60%, in oil, 240 mg) at 0° C. and the mixture was stirred overnight at room temperature. The reaction mixture was poured into water, and extracted with ethyl acetate. The ethyl acetate layer was washed with dilute hydrochloric acid and then with saturated aqueous sodium chloride solution, dried (MgSO₄) and concentrated. The residue was subjected to silica gel column chromatography, and ethyl (E)-3-(1-methyl-3-{3-methyl-1-[5-(trifluoromethyl)-2-pyridyl]-1H-pyrazol-4-ylmethoxy}-1H-pyrazol-5-yl)propenoate (2.14 g, yield 80%) was obtained as colorless crystals from a fraction eluted with ethyl acetate-hexane (1:4, volume ratio). The crystals were recrystallized from ethyl acetate-hexane. melting point: 173-174° C.

¹H-NMR (CDCl₃)δ: 1.33 (3H, t, J=7.2 Hz), 2.40 (3H, s), 3.83 (3H, s), 4.26 (2H, q, J=7.2 Hz), 5.11 (2H, s), 5.94 (1H, s), 6.27 (1H, d, J=15.9 Hz), 7.47 (1H, d, J=15.9 Hz), 7.94-8.04 (2H, m), 8.57 (1H, s), 8.60-8.65 (1H, m).

### Example 37

A mixture of ethyl (E)-3-(1-methyl-3-{3-methyl-1-[5-(trifluoromethyl)-2-pyridyl]-1H-pyrazol-4-ylmethoxy}-1H-pyrazol-5-yl)propenoate (600 mg), 1N aqueous sodium hydroxide solution (5 ml), tetrahydrofuran (5 ml) and methanol (5 ml) was stirred at 60° C. for 2 hours. 1N Hydrochloric acid (10 ml) was added and the mixture was extracted with ethyl acetate. The ethyl acetate layer was washed with saturated aqueous sodium chloride solution, dried (MgSO₄) and concentrated. The obtained colorless crystals were collected by filtration to give (E)-3-(1-methyl-3-{3-methyl-1-[5-(trifluoromethyl)-2-pyridyl]-1H-pyrazol-4-ylmethoxy}-1H-pyrazol-5-yl)propenoic acid (520 mg, yield 93%). The crystals were recrystallized from acetone-hexane. melting point: 208-209° C.

¹H-NMR (CDCl₃)δ: 2.41 (3H, s), 3.85 (3H, s), 5.13 (2H, s), 6.00 (1H, s), 6.28 (1H, d, J=15.8 Hz), 7.57 (1H, d, J=15.8 Hz), 7.93-8.07 (2H, m), 8.58 (1H, s), 8.60-8.66 (1H, m).

### Example 38

A mixture of ethyl (E)-3-(1-methyl-3-{3-methyl-1-[5-(trifluoromethyl)-2-pyridyl]-1H-pyrazol-4-ylmethoxy}-1H-pyrazol-5-yl)propenoate (1.25 g), 5% palladium-carbon (600 mg) and tetrahydrofuran (30 ml) was stirred at room temperature for 1 hour under a hydrogen atmosphere. Palladium-carbon was removed by filtration and the filtrate was concentrated. A mixture of the obtained crystals, 1N aqueous sodium hydroxide solution (5 ml), tetrahydrofuran (5 ml) and methanol (5 ml) was stirred at room temperature for 3 hours. 1N Hydrochloric acid (5 ml) was added and the mixture was extracted with ethyl acetate. The ethyl acetate layer was washed with saturated aqueous sodium chloride solution, dried (MgSO₄) and concentrated. The obtained colorless crystals were collected by filtration to give 3-(1-methyl-3-{3-methyl-1-[5-(trifluoromethyl)-2-pyridyl]-1H-pyrazol-4-ylmethoxy}-1H-pyrazol-5-yl)propionic acid (1.13 g, yield 96%). The crystals were recrystallized from acetone-hexane. melting point: 154-155° C.

¹H-NMR (CDCl₃)δ: 2.39 (3H, s), 2.64-2.77 (2H, m), 2.81-2.94 (2H, m), 3.68 (3H, s), 5.07 (2H, s), 5.51 (1H, s), 7.94-8.07 (2H, m), 8.54 (1H, s), 8.60-8.65 (1H, m).

### Example 39

To a mixture of {3-isopropyl-1-[5-(trifluoromethyl)-2-pyridyl]-1H-pyrazol-4-yl}methanol (1.50 g), methyl 3-hydroxy-1-methyl-1H-pyrazole-5-carboxylate (830 mg), triphenylphosphine (1.40 g) and tetrahydrofuran (30 ml) was dropwise added a 40% solution (2.35 g) of diethyl azodicarboxylate in toluene at room temperature and the mixture was stirred overnight. The reaction solution was concentrated. The residue was subjected to silica gel column chromatography, and methyl 3-{3-isopropyl-1-[5-(trifluoromethyl)-2-pyridyl]-1H-pyrazol-4-ylmethoxy}-1-methyl-1H-pyrazole-5-carboxylate (2.00 g, yield 90%) was obtained as colorless crystals from a fraction eluted with ethyl acetate-hexane (1:4, volume ratio). The crystals were recrystallized from ethyl acetate-hexane. melting point: 114-115° C.

¹H-NMR (CDCl₃)δ: 1.36 (6H, d, J=6.9 Hz), 3.10-3.24 (1H, m), 3.87 (3H, s), 4.06 (3H, s), 5.15 (2H, s), 6.21 (1H, s), 7.94-8.10 (2H, m), 8.57 (1H, s), 8.61-8.66 (1H, m).

### Example 40

To a mixture of {3-methyl-1-[5-(trifluoromethyl)-2-pyridyl]-1H-pyrazol-4-yl}methanol (3.95 g), methyl 3-hydroxy-1-methyl-1H-pyrazole-5-carboxylate (2.39 g), triphenylphosphine (4.50 g) and tetrahydrofuran (50 ml) was dropwise added a 40% solution (7.60 g) of diethyl azodicarboxylate in toluene at room temperature and the mixture was stirred overnight. The reaction solution was concentrated. The residue was subjected to silica gel column chromatography, and methyl 1-methyl-3-{3-methyl-1-[5-(trifluoromethyl)-2-pyridyl]-1H-pyrazol-4-ylmethoxy}-1H-pyrazole-5-carboxylate (4.90 g, yield 81%) was obtained as colorless crystals from a fraction eluted with ethyl acetate-hexane (1:4, volume ratio). The crystals were recrystallized from ethyl acetate-hexane. melting point: 130-131° C.

¹H-NMR (CDCl₃)δ: 2.40 (3H, s), 3.86 (3H, s), 4.05 (3H, s), 5.12 (2H, s), 6.20 (1H, s), 7.94-8.06 (2H, m), 8.57 (1H, s), 8.59-8.67 (1H, m).

### Example 41

To a mixture of 3-{3-propyl-1-[5-(trifluoromethyl)-2-pyridyl]-1H-pyrazol-4-yl}-1-propanol (0.40 g), methyl 2-(3-hydroxyphenoxy)-2-methylpropionate (280 mg), tributylphosphine (500 mg) and tetrahydrofuran (10 ml) was added 1,1′-azodicarbonyldipiperidine (630 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours. 1N Hydrochloric acid (5 ml) was added and the mixture was extracted with ethyl acetate. The ethyl acetate layer was washed with saturated aqueous sodium chloride solution, dried (MgSO₄) and concentrated. The obtained colorless crystals were collected by filtration to give 2-methyl-2-[3-(3-{3-propyl-1-[5-(trifluoromethyl)-2-pyridyl]-1H-pyrazol-4-yl}propoxy)phenoxy]propionic acid (300 mg, yield 48%). The crystals were recrystallized from ethyl acetate-hexane. melting point: 99-100° C.

¹H-NMR (CDCl₃)δ: 1.00 (3H, t, J=7.0 Hz), 1.61 (6H, s), 1.60-1.83 (2H m), 1.98-2.10 (2H, m), 2.55-2.76 (4H, m), 3.98 (2H, t, J=6.2 Hz), 6.50-6.70 (3H, m), 7.11-7.24 (1H, m), 7.90-8.08 (2H, m), 8.27 (1H, s), 8.55-8.64 (1H, m).

### Example 42

To a mixture of 3-{3-ethoxy-1-[5-(trifluoromethyl)-2-pyridyl]-1H-pyrazol-4-yl}-1-propanol (500 mg), ethyl 3-(3-hydroxy-1-phenyl-1H-pyrazol-5-yl)propionate (440 mg), tributylphosphine (650 mg) and tetrahydrofuran (10 ml) was added 1,1′-azodicarbonyldipiperidine (810 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and ethanol (5 ml) was stirred at room temperature for 5 hours. 1N Hydrochloric acid (5 ml) was added and the mixture was extracted with ethyl acetate. The ethyl acetate layer was washed with saturated aqueous sodium chloride solution, dried (MgSO₄) and concentrated. The obtained colorless crystals were collected by filtration to give 3-[3-(3-{3-ethoxy-1-[5-(trifluoromethyl)-2-pyridyl]-1H-pyrazol-4-yl}propoxy)-1-phenyl-1H-pyrazol-5-yl]propionic acid (460 mg, yield 55%). The crystals were recrystallized from ethyl acetate-hexane. melting point: 121-122° C.

¹H-NMR (CDCl₃)δ: 1.42 (3H, t, J=7.0 Hz), 1.96-2.18 (2H, m), 2.52-2.71 (4H, m), 2.88-3.00 (2H, m), 4.17-4.28 (2H, m), 4.35 (2H, q, J=7.0 Hz), 5.71 (1H, s), 7.27-7.50 (5H, m), 7.76-7.95 (2H, m), 8.17 (1H, s), 8.50-8.56 (1H, m).

### Example 43

To a mixture of 3-{3-propyl-1-[5-(trifluoromethyl)-2-pyridyl]-1H-pyrazol-4-yl}-1-propanol (540 mg), ethyl 3-(3-hydroxy-1-phenyl-1H-pyrazol-5-yl)propionate (450 mg), tributylphosphine (700 mg) and tetrahydrofuran (20 ml) was added 1,1′-azodicarbonyldipiperidine (860 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours. 1N Hydrochloric acid (5 ml) was added and the mixture was extracted with ethyl acetate. The ethyl acetate layer was washed with saturated aqueous sodium chloride solution, dried (MgSO₄) and concentrated. The obtained colorless crystals were collected by filtration to give 3-[1-phenyl-3-(3-{3-propyl-1-[5-(trifluoromethyl)-2-pyridyl]-1H-pyrazol-4-yl}propoxy)-1H-pyrazol-5-yl]propionic acid (630 mg, yield 69%). The crystals were recrystallized from ethyl acetate-hexane. melting point: 149-150° C.

¹H-NMR (CDCl₃)δ: 1.00 (3H, t, J=7.2 Hz), 1.62-1.85 (2H, m), 1.98-2.18 (2H, m), 2.55-2.71 (6H, m), 2.88-3.02 (2H, m), 4.18-4.30 (2H, m), 5.71 (1H, s), 7.27-7.51 (5H, m), 7.89-8.06 (2H, m), 8.29 (1H, s), 8.55-8.62 (1H, m).

### Example 44

To a mixture of 3-{3-propyl-1-[5-(trifluoromethyl)-2-pyridyl]-1H-pyrazol-4-yl}-1-propanol (550 mg), methyl 3-hydroxyphenylacetate (300 mg), tributylphosphine (740 mg) and tetrahydrofuran (20 ml) was added 1,1′-azodicarbonyldipiperidine (890 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours. 1N Hydrochloric acid (5 ml) was added and the mixture was extracted with ethyl acetate. The ethyl acetate layer was washed with saturated aqueous sodium chloride solution, dried (MgSO₄) and concentrated. The obtained colorless crystals were collected by filtration to give [3-(3-{3-propyl-1-[5-(trifluoromethyl)-2-pyridyl]-1H-pyrazol-4-yl}propoxy)phenyl]acetic acid (630 mg, yield 80%). The crystals were recrystallized from ethyl acetate-hexane. melting point: 106-107° C.

¹H-NMR (CDCl₃)δ: 1.00 (3H, t, J=7.0 Hz), 1.62-1.82 (2H m) 2.00-2.18 (2H, m), 2.55-2.74 (4H, m), 3.62 (2H, s), 4.03 (2H, t, J=6.2 Hz), 6.70-6.92 (3H, m), 7.17-7.32 (1H, m), 7.90-8.05 (2H, m), 8.30 (1H, s), 8.58-8.64 (1H, m).

### Example 45

To a mixture of 3-{3-propyl-1-[5-(trifluoromethyl)-2-pyridyl]-1H-pyrazol-4-yl}-1-propanol (500 mg), ethyl 3-(3-hydroxy-1-methyl-1H-pyrazol-5-yl)propionate (320 mg), tributylphosphine (650 mg) and tetrahydrofuran (20 ml) was added 1,1′-azodicarbonyldipiperidine (800 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and ethanol (5 ml) was stirred at room temperature for 5 hours. 1N Hydrochloric acid (5 ml) was added and the mixture was extracted with ethyl acetate. The ethyl acetate layer was washed with saturated aqueous sodium chloride solution, dried (MgSO₄) and concentrated. The obtained colorless crystals were collected by filtration to give 3-[1-methyl-3-(3-{3-propyl-1-[5-(trifluoromethyl)-2-pyridyl]-1H-pyrazol-4-yl}propoxy)-1H-pyrazol-5-yl]propionic acid (550 mg, yield 74%). The crystals were recrystallized from ethyl acetate-hexane. melting point: 80-81° C.

¹H-NMR (CDCl₃)δ: 1.00 (3H, t, J=7.4 Hz), 1.60-1.84 (2H, m), 1.95-2.14 (2H, m), 2.54-2.93 (8H, m), 3.66 (3H, s), 4.08-4.20 (2H, m), 5.48 (1H, s), 7.90-8.06 (2H, m), 8.28 (1H, s), 8.57-8.64 (1H, m).

### Example 46

To a mixture of 3-{3-propyl-1-[5-(trifluoromethyl)-2-pyridyl]-1H-pyrazol-4-yl}-1-propanol (550 mg), methyl 4-hydroxyphenylacetate (300 mg), tributylphosphine (750 mg) and tetrahydrofuran (30 ml) was added 1,1′-azodicarbonyldipiperidine (890 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours. 1N Hydrochloric acid (5 ml) was added and the mixture was extracted with ethyl acetate. The ethyl acetate layer was washed with saturated aqueous sodium chloride solution, dried (MgSO₄) and concentrated. The obtained colorless crystals were collected by filtration to give [4-(3-{3-propyl-1-[5-(trifluoromethyl)-2-pyridyl]-1H-pyrazol-4-yl}propoxy)phenyl]acetic acid (590 mg, yield 75%). The crystals were recrystallized from ethyl acetate-hexane. melting point: 101-102° C.

¹H-NMR (CDCl₃)δ: 1.00 (3H, t, J-7.4 Hz), 1.62-1.84 (2H m) 2.01-2.19 (2H, m), 2.55-2.73 (4H, m), 3.60 (2H, s), 3.96-4.06 (2H, m), 6.82-6.92 (2H, m), 7.14-7.24 (2H, m), 7.90-8.06 (2H, m), 8.30 (1H, s), 8.57-8.64 (1H, m).

### Example 47

To a mixture of 3-{3-propyl-1-[5-(trifluoromethyl)-2-pyridyl]-1H-pyrazol-4-yl}-1-propanol (550 mg), methyl 2-hydroxyphenylacetate (300 mg), tributylphosphine (750 mg) and tetrahydrofuran (30 ml) was added 1,1′-azodicarbonyldipiperidine (900 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours. 1N Hydrochloric acid (5 ml) was added and the mixture was extracted with ethyl acetate. The ethyl acetate layer was washed with saturated aqueous sodium chloride solution, dried (MgSO₄) and concentrated. The obtained colorless crystals were collected by filtration to give [2-(3-{3-propyl-1-[5-(trifluoromethyl)-2-pyridyl]-1H-pyrazol-4-yl}propoxy)phenyl]acetic acid (620 mg, yield 79%). The crystals were recrystallized from ethyl acetate-hexane. melting point: 100-101° C.

### Example 48

To a mixture of 3-{3-ethoxy-1-[5-(trifluoromethyl)-2-pyridinyl]-1H-pyrazol-4-yl}-1-propanol (500 mg), ethyl 3-(3-hydroxy-1-methyl-1H-pyrazol-5-yl)propanoate (346 mg), tributylphosphine (790 μL) and tetrahydrofuran (53 ml) was added 1,1′-azodicarbonyldipiperidine (800 mg) at room temperature and the mixture was stirred overnight. The reaction solution was concentrated. The residue was subjected to silica gel column chromatography, and a colorless oil was obtained from a fraction eluted with ethyl acetate-hexane (1:2, volume ratio). A mixture of the obtained oily substance, 1N aqueous sodium hydroxide solution (25 ml), tetrahydrofuran (50 ml) and ethanol (25 ml) was stirred at room temperature for 3 hours. 1N Hydrochloric acid (25 ml) was added and the mixture was extracted with ethyl acetate. The ethyl acetate layer was washed with saturated aqueous sodium chloride solution, dried (MgSO₄) and concentrated. The obtained colorless crystals were recrystallized from ethyl acetate-hexane to give 3-[3-(3-{3-ethoxy-1-[5-(trifluoromethyl)-2-pyridinyl]-1H-pyrazol-4-yl}propoxy)-1-methyl-1H-pyrazol-5-yl]propanoic acid (370 mg, yield 50%). melting point: 137-138° C.

### Example 49

To a mixture of 4-{3-isopropyl-1-[5-(trifluoromethyl)-2-pyridinyl]-1H-pyrazol-4-yl}-1-butanol (500 mg), ethyl 3-(3-hydroxy-1-phenyl-1H-pyrazol-5-yl)propanoate (437 mg), tributylphosphine (761 μL) and tetrahydrofuran (50 ml) was added 1,1′-azodicarbonyldipiperidine (771 mg) at room temperature and the mixture was stirred overnight. The reaction solution was concentrated. The residue was subjected to silica gel column chromatography, and a colorless oil was obtained from a fraction eluted with ethyl acetate-hexane (1:5, volume ratio). A mixture of the obtained oily substance, 1N aqueous sodium hydroxide solution (25 ml), tetrahydrofuran (50 ml) and ethanol (25 ml) was stirred at room temperature for 2 hours. 1N Hydrochloric acid (25 ml) was added and the mixture was extracted with ethyl acetate. The ethyl acetate layer was washed with saturated aqueous sodium chloride solution, dried (MgSO₄) and concentrated. The obtained colorless crystals were recrystallized from ethyl acetate-hexane to give 3-[3-(4-{3-isopropyl-1-[5-(trifluoromethyl)-2-pyridinyl]-1H-pyrazol-4-yl}butoxy)-1-phenyl-1H-pyrazol-5-yl]propanoic acid (594 mg, yield 72%) melting point: 137-138° C.

### Example 50

To a mixture of 4-{3-isopropyl-1-[5-(trifluoromethyl)-2-pyridinyl]-1H-pyrazol-4-yl}-1-butanol (500 mg), ethyl 3-(3-hydroxy-1-methyl-1H-pyrazol-5-yl)propanoate (333 mg), tributylphosphine (761 μL) and tetrahydrofuran (50 ml) was added 1,1′-azodicarbonyldipiperidine (771 mg) at room temperature and the mixture was stirred overnight. The reaction solution was concentrated. The residue was subjected to silica gel column chromatography, and a colorless oil was obtained from a fraction eluted with ethyl acetate-hexane (1:5, volume ratio). A mixture of the obtained oily substance, 1N aqueous sodium hydroxide solution (25 ml), tetrahydrofuran (50 ml) and ethanol (25 ml) was stirred at room temperature for 2 hours. 1N Hydrochloric acid (25 ml) was added and the mixture was extracted with ethyl acetate. The ethyl acetate layer was washed with saturated aqueous sodium chloride solution, dried (MgSO₄) and concentrated. The obtained colorless crystals were recrystallized from ethyl acetate-hexane to give 3-[3-(4-{3-isopropyl-1-[5-(trifluoromethyl)-2-pyridinyl]-1H-pyrazol-4-yl}butoxy)-1-methyl-1H-pyrazol-5-yl]propanoic acid (366 mg, yield 50%). melting point: 113-114° C.

### Example 51

To a mixture of 4-{3-isopropyl-1-[5-(trifluoromethyl)-2-pyridinyl]-1H-pyrazol-4-yl}-1-butanol (500 mg), methyl 3-hydroxyphenylacetate (279 mg), tributylphosphine (761 μL) and tetrahydrofuran (50 ml) was added 1,1′-azodicarbonyldipiperidine (771 mg) at room temperature and the mixture was stirred overnight. The reaction solution was concentrated. The residue was subjected to silica gel column chromatography, and a colorless oil was obtained from a fraction eluted with ethyl acetate-hexane (1:20, volume ratio). A mixture of the obtained oily substance, 1N aqueous sodium hydroxide solution (25 ml), tetrahydrofuran (50 ml) and ethanol (25 ml) was stirred at room temperature for 6 hours. 1N Hydrochloric acid (25 ml) was added and the mixture was extracted with ethyl acetate. The ethyl acetate layer was washed with saturated aqueous sodium chloride solution, dried (MgSO₄) and concentrated. The obtained colorless crystals were recrystallized from diisopropyl ether-hexane, [3-(4-{3-isopropyl-1-[5-(trifluoromethyl)-2-pyridinyl]-1H-pyrazol-4-yl}butoxy)phenyl]acetic acid (165 mg, yield 23%). melting point: 114-115° C.

### Example 52

To a mixture of 4-{3-isopropyl-1-[5-(trifluoromethyl)-2-pyridinyl]-1H-pyrazol-4-yl}-1-butanol (500 mg), methyl 2-hydroxyphenylacetate (279 mg), tributylphosphine (761 μL) and tetrahydrofuran (80 ml) was added 1,1′-azodicarbonyldipiperidine (771 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (25 ml), tetrahydrofuran (50 ml) and ethanol (25 ml) was stirred at room temperature for 2 hours. 1N Hydrochloric acid (25 ml) was added and the mixture was extracted with ethyl acetate. The ethyl acetate layer was washed with saturated aqueous sodium chloride solution, dried (MgSO₄) and concentrated. The obtained colorless crystals were recrystallized from ethyl acetate-hexane to give [2-(4-{3-isopropyl-1-[5-(trifluoromethyl)-2-pyridinyl]-1H-pyrazol-4-yl}butoxy)phenyl]acetic acid (376 mg, yield 53%). melting point: 125-126° C.

### Example 53

To a mixture of 4-{3-isopropyl-1-[5-(trifluoromethyl)-2-pyridinyl]-1H-pyrazol-4-yl}-1-butanol (500 mg), methyl 4-hydroxyphenylacetate (279 mg), tributylphosphine (761 μL) and tetrahydrofuran (76 ml) was added 1,1′-azodicarbonyldipiperidine (771 mg) at room temperature and the mixture was stirred overnight. The reaction solution was concentrated. The residue was subjected to silica gel column chromatography, and a colorless oil was obtained from a fraction eluted with ethyl acetate-hexane (1:9, volume ratio). A mixture of the obtained oily substance, 1N aqueous sodium hydroxide solution (50 ml), tetrahydrofuran (50 ml) and ethanol (25 ml) was stirred at room temperature for 4 hours. 1N Hydrochloric acid (50 ml) was added and the mixture was extracted with ethyl acetate. The ethyl acetate layer was washed with saturated aqueous sodium chloride solution, dried (MgSO₄) and concentrated. The obtained colorless crystals were recrystallized from ethyl acetate-hexane to give [4-(4-{3-isopropyl-1-[5-(trifluoromethyl)-2-pyridinyl]-1H-pyrazol-4-yl}butoxy)phenyl]acetic acid (335 mg, yield 47%). melting point: 130-131° C.

### Example 54

To a mixture of 4-{3-isopropyl-1-[5-(trifluoromethyl)-2-pyridinyl]-1H-pyrazol-4-yl}-1-butanol (500 mg), methyl 2-(3-hydroxyphenoxy)-2-methylpropanoate (353 mg), tributylphosphine (761 μL) and tetrahydrofuran (76 ml) was added 1,1′-azodicarbonyldipiperidine (771 mg) at room temperature and the mixture was stirred overnight. The reaction solution was concentrated. The residue was subjected to silica gel column chromatography, and a colorless oil was obtained from a fraction eluted with ethyl acetate-hexane (1:9, volume ratio). A mixture of the obtained oily substance, 1N aqueous sodium hydroxide solution (50 ml), tetrahydrofuran (50 ml) and ethanol (25 ml) was stirred at room temperature for 3 hours. 1N Hydrochloric acid (50 ml) was added and the mixture was extracted with ethyl acetate. The ethyl acetate layer was washed with saturated aqueous sodium chloride solution, dried (MgSO₄) and concentrated. The residue was subjected to silica gel column chromatography, and colorless crystals were obtained from a fraction eluted with ethyl acetate-hexane (1:1, volume ratio). The obtained colorless crystals were recrystallized from ethyl acetate-hexane to give 2-[3-(4-{3-isopropyl-1-[5-(trifluoromethyl)-2-pyridinyl]-1H-pyrazol-4-yl}butoxy)phenoxy]-2-methylpropanoic acid (258 mg, yield 33%). melting point: 81-82° C.

### Example 55

To a mixture of 4-{3-isopropyl-1-[5-(trifluoromethyl)-2-pyridinyl]-1H-pyrazol-4-yl}-1-butanol (500 mg), methyl 3-(4-hydroxyphenyl)propanoate (303 mg), tributylphosphine (761 μL) and tetrahydrofuran (76 ml) was added 1,1′-azodicarbonyldipiperidine (771 mg) at room temperature and the mixture was stirred overnight. The reaction solution was concentrated. The residue was subjected to silica gel column chromatography, and a colorless oil was obtained from a fraction eluted with ethyl acetate-hexane (1:5, volume ratio). mixture of the obtained oily substance, 1N aqueous sodium hydroxide solution (50 ml), tetrahydrofuran (50 ml) and ethanol (25 ml) was stirred at room temperature for 3 hours. 1N Hydrochloric acid (50 ml) was added and the mixture was extracted with ethyl acetate. The ethyl acetate layer was washed with saturated aqueous sodium chloride solution, dried (MgSO₄) and concentrated. The obtained colorless crystals were recrystallized from ethyl acetate-hexane to give 3-[4-(4-{3-isopropyl-1-[5-(trifluoromethyl)-2-pyridinyl]-1H-pyrazol-4-yl}butoxy)phenyl]propanoic acid (231 mg, yield 32%). melting point: 144-145° C.

### Example 56

To a mixture of 2-{3-ethoxy-1-[5-(trifluoromethyl)-2-pyridinyl]-1H-pyrazol-4-yl}ethanol (300 mg), ethyl 3-(3-hydroxy-1-phenyl-1H-pyrazol-5-yl)propanoate (285 mg), tributylphosphine (496 μL) and tetrahydrofuran (50 ml) was added 1,1′-azodicarbonyldipiperidine (503 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (30 ml), tetrahydrofuran (30 ml) and ethanol (15 ml) was stirred at room temperature for 5 hours. 1N Hydrochloric acid (30 ml) was added and the mixture was extracted with ethyl acetate. The ethyl acetate layer was washed with saturated aqueous sodium chloride solution, dried (MgSO₄) and concentrated. The obtained colorless crystals were recrystallized from ethyl acetate-hexane to give 3-[3-(2-{3-ethoxy-1-[5-(trifluoromethyl)-2-pyridinyl]-1H-pyrazol-4-yl}ethoxy)-1-phenyl-1H-pyrazol-5-yl]propanoic acid (372 mg, yield 72%). melting point: 155-156° C.

### Example 57

To a mixture of 2-{3-ethoxy-1-[5-(trifluoromethyl)-2-yridinyl]-1H-pyrazol-4-yl}ethanol (300 mg), methyl 2-hydroxyphenylacetate (183 mg), tributylphosphine (496 μL) and tetrahydrofuran (50 ml) was added 1,1′-azodicarbonyldipiperidine (502 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (25 ml), tetrahydrofuran (25 ml) and ethanol (25 ml) was stirred at room temperature for 3 hours. 1N Hydrochloric acid (25 ml) was added and the mixture was extracted with ethyl acetate. The ethyl acetate layer was washed with saturated aqueous sodium chloride solution, dried (MgSO₄) and concentrated. The obtained colorless crystals were recrystallized from ethyl acetate-hexane to give [2-(2-{3-ethoxy-1-[5-(trifluoromethyl)-2-pyridinyl]-1H-pyrazol-4-yl}ethoxy)phenyl]acetic acid (242 mg, yield 56%). melting point: 134-135° C.

### Example 58

To a mixture of 4-{3-propyl-1-[5-(trifluoromethyl)-2-pyridinyl]-1H-pyrazol-4-yl}-1-butanol (500 mg), ethyl 3-(3-hydroxy-1-phenyl-1H-pyrazol-5-yl)propanoate (437 mg), tributylphosphine (761 μL) and tetrahydrofuran (76 ml) was added 1,1′-azodicarbonyldipiperidine (771 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (30 ml), tetrahydrofuran (30 ml) and ethanol (30 ml) was stirred at room temperature for 3 hours. 1N Hydrochloric acid (30 ml) was added and the mixture was extracted with ethyl acetate. The ethyl acetate layer was washed with saturated aqueous sodium chloride solution, dried (MgSO₄) and concentrated. The obtained colorless crystals were recrystallized from ethyl acetate-hexane to give 3-[1-phenyl-3-(4-{3-propyl-1-[5-(trifluoromethyl)-2-pyridinyl]-1H-pyrazol-4-yl}butoxy)-1H-pyrazol-5-yl]propanoic acid (505 mg, yield 61%). melting point: 123-124° C.

### Example 59

To a mixture of 4-{3-propyl-1-[5-(trifluoromethyl)-2-pyridinyl]-1H-pyrazol-4-yl}-1-butanol (500 mg), methyl 3-hydroxyphenylacetate (508 mg), tributylphosphine (761 μL) and tetrahydrofuran (76 ml) was added 1,1′-azodicarbonyldipiperidine (771 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (30 ml), tetrahydrofuran (30 ml) and ethanol (30 ml) was stirred at room temperature for 3.5 hours. 1N Hydrochloric acid (30 ml) was added and the mixture was extracted with ethyl acetate. The ethyl acetate layer was washed with saturated aqueous sodium chloride solution, dried (MgSO₄) and concentrated. The obtained colorless crystals were recrystallized from ethyl acetate-hexane to give [3-(4-{3-propyl-1-[5-(trifluoromethyl)-2-pyridinyl]-1H-pyrazol-4-yl}butoxy)phenyl]acetic acid (330 mg, yield 47%). melting point: 96-97° C.

### Example 60

To a mixture of 4-{3-propyl-1-[5-(trifluoromethyl)-2-pyridinyl]-1H-pyrazol-4-yl}-1-butanol (500 mg), methyl 2-hydroxyphenylacetate (279 mg), tributylphosphine (761 μL) and tetrahydrofuran (76 ml) was added 1,1′-azodicarbonyldipiperidine (771 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (30 ml), tetrahydrofuran (30 ml) and ethanol (30 ml) was stirred at room temperature for 3 hours. 1N Hydrochloric acid (30 ml) was added and the mixture was extracted with ethyl acetate. The ethyl acetate layer was washed with saturated aqueous sodium chloride solution, dried (MgSO₄) and concentrated. The obtained colorless crystals were recrystallized from ethyl acetate-hexane to give [2-(4-{3-propyl-1-[5-(trifluoromethyl)-2-pyridinyl]-1H-pyrazol-4-yl}butoxy)phenyl]acetic acid (236 mg, yield 33%). melting. point: 95-97° C.

### Example 61

To a mixture of 4-{3-propyl-1-[5-(trifluoromethyl)-2-pyridinyl]-1H-pyrazol-4-yl}-1-butanol (500 mg), methyl (3-hydroxy-1-methyl-1H-pyrazol-4-yl)acetate (286 mg), tributylphosphine (761 μL) and tetrahydrofuran (76 ml) was added 1,1′-azodicarbonyldipiperidine (771 mg) at room temperature and the mixture was stirred overnight. The reaction solution was concentrated. The residue was subjected to silica gel column chromatography, and a colorless oil was obtained from a fraction eluted with ethyl acetate-hexane (1:3, volume ratio). A mixture of the obtained oily substance, 1N aqueous sodium hydroxide solution (30 ml), tetrahydrofuran (30 ml) and ethanol (30 ml) was stirred at room temperature for 4 hours. 1N Hydrochloric acid (30 ml) was added and the mixture was extracted with ethyl acetate. The ethyl acetate layer was washed with saturated aqueous sodium chloride solution, dried (MgSO₄) and concentrated. The obtained colorless crystals were recrystallized from ethyl acetate-hexane to give [1-methyl-3-(4-{3-propyl-1-[5-(trifluoromethyl)-2-pyridinyl]-1H-pyrazol-4-yl}butoxy)-1H-pyrazol-4-yl]acetic acid (340 mg, yield 48%). melting point: 95-97° C.

### Example 62

To a mixture of 2-{3-ethoxy-1-[5-(trifluoromethyl)-2-pyridinyl]-1H-pyrazol-4-yl}ethanol (460 mg), methyl 3-hydroxyphenylacetate (507 mg), tributylphosphine (761 μL) and tetrahydrofuran (76 ml) was added 1,1′-azodicarbonyldipiperidine (771 mg) at room temperature and the mixture was stirred overnight. The reaction solution was concentrated. The residue was subjected to silica gel column chromatography, and a colorless oil was obtained from a fraction eluted with ethyl acetate-hexane (1:5, volume ratio). A mixture of the obtained oily substance, 1N aqueous sodium hydroxide solution (30 ml), tetrahydrofuran (30 ml) and ethanol (30 ml) was stirred at room temperature for 3 hours. 1N Hydrochloric acid (30 ml) was added and the mixture was extracted with ethyl acetate. The ethyl acetate layer was washed with saturated aqueous sodium chloride solution, dried (MgSO₄) and concentrated. The residue was subjected to silica gel column chromatography, and colorless crystals were obtained from a fraction eluted with ethyl acetate-hexane (1:2, volume ratio). The obtained colorless crystals were recrystallized from ethyl acetate-hexane to give [3-(2-{3-ethoxy-1-[5-(trifluoromethyl)-2-pyridinyl]-1H-pyrazol-4-yl}ethoxy)phenyl]acetic acid (206 mg, yield 31%). melting point: 128-130° C.

### Example 63

To a mixture of 4-{3-propyl-1-[5-(trifluoromethyl)-2-pyridinyl]-1H-pyrazol-4-yl}-1-butanol (500 mg), methyl 3-hydroxy-4-methoxyphenylacetate (899 mg), tributylphosphine (1.14 ml) and tetrahydrofuran (76 ml) was added 1,1′-azodicarbonyldipiperidine (1.16 g) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (30 ml), tetrahydrofuran (30 ml) and ethanol (30 ml) was stirred at room temperature for 3 hours. 1N Hydrochloric acid (30 ml) was added and the mixture was extracted with ethyl acetate. The ethyl acetate layer was washed with saturated aqueous sodium chloride solution, dried (MgSO₄) and concentrated. The obtained colorless crystals were recrystallized from ethyl acetate-hexane to give [4-methoxy-3-(4-{3-propyl-1-[5-(trifluoromethyl)-2-pyridinyl]-1H-pyrazol-4-yl}butoxy)phenyl]acetic acid (388 mg, yield 52%). melting point: 147-148° C.

### Example 64

To a mixture of 4-{3-propyl-1-[5-(trifluoromethyl)-2-pyridinyl]-1H-pyrazol-4-yl}-1-butanol (500 mg), ethyl 3-(4-hydroxy-2-methylphenyl)propanoate (350 mg), tributylphosphine (761 μL) and tetrahydrofuran (76 ml) was added 1,1′-azodicarbonyldipiperidine (771 mg) at room temperature and the mixture was stirred overnight. The reaction solution was concentrated. The residue was subjected to silica gel column chromatography, and a colorless oil was obtained from a fraction eluted with ethyl acetate-hexane (1:5, volume ratio). A mixture of the obtained oily substance, 1N aqueous sodium hydroxide solution (30 ml), tetrahydrofuran (30 ml) and ethanol (30 ml) was stirred at room temperature for 3 hours. 1N Hydrochloric acid (30 ml) was added and the mixture was extracted with ethyl acetate. The ethyl acetate layer was washed with saturated aqueous sodium chloride solution, dried (MgSO₄) and concentrated. The obtained colorless crystals were recrystallized from ethyl acetate-hexane to give 3-[2-methyl-4-(4-{3-propyl-1-[5-(trifluoromethyl)-2-pyridinyl]-1H-pyrazol-4-yl}butoxy)phenyl]propanoic acid (323 mg, yield 43%). melting point: 105-107° C.

### Example 65

To a mixture of 3-{3-propyl-1-[5-(trifluoromethyl)-2-pyridinyl]-1H-pyrazol-4-yl}-1-propanol (480 mg), ethyl 3-(4-hydroxy-2-methylphenyl)propanoate (351 mg), tributylphosphine (763 μL) and tetrahydrofuran (76 ml) was added 1,1′-azodicarbonyldipiperidine (773 mg) at room temperature and the mixture was stirred overnight. The reaction solution was concentrated. The residue was subjected to silica gel column chromatography, and a colorless oil was obtained from a fraction eluted with ethyl acetate-hexane (1:5, volume ratio). A mixture of the obtained oily substance, 1N aqueous sodium hydroxide solution (30 ml), tetrahydrofuran (30 ml) and ethanol (30 ml) was stirred at room temperature overnight. 1N Hydrochloric acid (30 ml) was added and the mixture was extracted with ethyl acetate. The ethyl acetate layer was washed with saturated aqueous sodium chloride solution, dried (MgSO₄) and concentrated. The obtained colorless crystals were recrystallized from ethyl acetate-hexane to give 3-[2-methyl-4-(3-{3-propyl-1-[5-(trifluoromethyl)-2-pyridinyl]-1H-pyrazol-4-yl}propoxy)phenyl]propanoic acid (147 mg, yield 20%). melting point: 124-126° C.

### Example 66

To a mixture of 4-{3-propyl-1-[5-(trifluoromethyl)-2-pyridinyl]-1H-pyrazol-4-yl}-1-butanol (500 mg), methyl (3-hydroxy-1-phenyl-1H-pyrazol-4-yl)acetate (390 mg), tributylphosphine (761 μL) and tetrahydrofuran (76 ml) was added 1,1′-azodicarbonyldipiperidine (771 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (30 ml), tetrahydrofuran (30 ml) and ethanol (30 ml) was stirred at room temperature overnight. 1N Hydrochloric acid (30 ml) was added and the mixture was extracted with ethyl acetate. The ethyl acetate layer was washed with saturated aqueous sodium chloride solution, dried (MgSO₄) and concentrated. The obtained colorless crystals were recrystallized from ethyl acetate-hexane to give [1-phenyl-3-(4-{3-propyl-1-[5-(trifluoromethyl)-2-pyridinyl]-1H-pyrazol-4-yl}butoxy)-1H-pyrazol-4-yl]acetic acid (600 mg, yield 74%). melting point: 114-115° C.

### Example 67

To a mixture of 3-{3-propyl-1-[5-(trifluoromethyl)-2-pyridinyl]-1H-pyrazol-4-yl}-1-propanol (480 mg), methyl (3-hydroxy-1-phenyl-1H-pyrazol-4-yl)acetate (391 mg), tributylphosphine (763 μL) and tetrahydrofuran (77 ml) was added 1,1′-azodicarbonyldipiperidine (773 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (30 ml), tetrahydrofuran (30 ml) and ethanol (30 ml) was stirred at room temperature for 3 hours. 1N Hydrochloric acid (30 ml) was added and the mixture was extracted with ethyl acetate. The ethyl acetate layer was washed with saturated aqueous sodium chloride solution, dried (MgSO₄) and concentrated. The obtained colorless crystals were recrystallized from ethyl acetate-hexane to give [1-phenyl-3-(3-{3-propyl-1-[5-(trifluoromethyl)-2-pyridinyl]-1H-pyrazol-4-yl}propoxy)-1H-pyrazol-4-yl]acetic acid (601 mg, yield 76%). melting point: 123-124° C.

### Example 68

To a mixture of 4-{3-isopropyl-1-[5-(trifluoromethyl)-2-pyridinyl]-1H-pyrazol-4-yl}-1-butanol (500 mg), methyl (3-hydroxy-1-phenyl-1H-pyrazol-4-yl)acetate (390 mg), tributylphosphine (761 μL) and tetrahydrofuran (76 ml) was added 1,1′-azodicarbonyldipiperidine (771 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (30 ml), tetrahydrofuran (30 ml) and ethanol (30 ml) was stirred at room temperature overnight. 1N Hydrochloric acid (30 ml) was added and the mixture was extracted with ethyl acetate. The ethyl acetate layer was washed with saturated aqueous sodium chloride solution, dried (MgSO₄) and concentrated. The obtained colorless crystals were recrystallized from ethyl acetate-hexane to give [3-(4-{3-isopropyl-1-[5-(trifluoromethyl)-2-pyridinyl]-1H-pyrazol-4-yl}butoxy)-1-phenyl-1H-pyrazol-4-yl]acetic acid (471 mg, yield 58%). melting point: 119-120° C.

### Example 69

To a mixture of 4-{3-isopropyl-1-[5-(trifluoromethyl)-2-pyridinyl]-1H-pyrazol-4-yl}-1-butanol (350 mg), methyl 3-(4-hydroxy-2-methoxyphenyl)propanoate (674 mg), tributylphosphine (799 μL) and tetrahydrofuran (53 ml) was added 1,1′-azodicarbonyldipiperidine (809 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (30 ml), tetrahydrofuran (30 ml) and ethanol (30 ml) was stirred at room temperature for 3 hours. 1N Hydrochloric acid (30 ml) was added and the mixture was extracted with ethyl acetate. The ethyl acetate layer was washed with saturated aqueous sodium chloride solution, dried (MgSO₄) and concentrated. The obtained colorless crystals were recrystallized from ethyl acetate-hexane to give 3-[4-(4-{3-isopropyl-1-[5-(trifluoromethyl)-2-pyridinyl]-1H-pyrazol-4-yl}butoxy)-2-methoxyphenyl]propanoic acid (319 mg, yield 59%). melting point: 125-126° C.

### Example 70

To a mixture of 4-{3-propyl-1-[5-(trifluoromethyl)-2-pyridinyl]-1H-pyrazol-4-yl}-1-butanol (500 mg), ethyl 3-(3-hydroxy-1-methyl-1H-pyrazol-5-yl)propanoate (333 mg), tributylphosphine (761 μL) and tetrahydrofuran (76 ml) was added 1,1′-azodicarbonyldipiperidine (771 mg) at room temperature and the mixture was stirred overnight. The reaction solution was concentrated. The residue was subjected to silica gel column chromatography, and a colorless oil was obtained from a fraction eluted with ethyl acetate-hexane (1:2, volume ratio). A mixture of the obtained oily substance, 1N aqueous sodium hydroxide solution (30 ml), tetrahydrofuran (30 ml) and ethanol (30 ml) was stirred at room temperature overnight. 1N Hydrochloric acid (30 ml) was added and the mixture was extracted with ethyl acetate. The ethyl acetate layer was washed with saturated aqueous sodium chloride solution, dried (MgSO₄) and concentrated. The obtained colorless crystals were recrystallized from ethyl acetate-hexane to give 3-[1-methyl-3-(4-{3-propyl-1-[5-(trifluoromethyl)-2-pyridinyl]-1H-pyrazol-4-yl}butoxy)-1H-pyrazol-5-yl]propanoic acid (345 mg, yield 47%). melting point: 122-123° C.

### Example 71

To a mixture of 3-{3-(benzyloxy)-1-[5-(trifluoromethyl)-2-pyridinyl]-1H-pyrazol-4-yl}-1-propanol (400 mg), ethyl 3-(3-hydroxy-1-phenyl-1H-pyrazol-5-yl)propanoate (247 mg), tributylphosphine (394 μL) and tetrahydrofuran (40 ml) was added 1,1′-azodicarbonyldipiperidine (399 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (30 ml), tetrahydrofuran (30 ml) and ethanol (30 ml) was stirred at room temperature for 3 hours. 1N Hydrochloric acid (30 ml) was added and the mixture was extracted with ethyl acetate. The ethyl acetate layer was washed with saturated aqueous sodium chloride solution, dried (MgSO₄) and concentrated. The obtained colorless crystals were recrystallized from ethyl acetate-hexane to give 3-[3-(3-{3-(benzyloxy)-1-[5-(trifluoromethyl)-2-pyridinyl]-1H-pyrazol-4-yl}propoxy)-1-phenyl-1H-pyrazol-5-yl]propanoic acid (378 mg, yield 81%). melting point: 159-161° C.

### Example 72

To a mixture of 2-{3-ethoxy-1-[5-(trifluoromethyl)-2-pyridinyl]-1H-pyrazol-4-yl}ethanol (400 mg), methyl (3-hydroxy-1-phenyl-1H-pyrazol-4-yl)acetate (339 mg), tributylphosphine (662 μL) and tetrahydrofuran (66 ml) was added 1,1′-azodicarbonyldipiperidine (670 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (30 ml), tetrahydrofuran (30 ml) and ethanol (30 ml) was stirred at room temperature overnight. 1N Hydrochloric acid (30 ml) was added and the mixture was extracted with ethyl acetate. The ethyl acetate layer was washed with saturated aqueous sodium chloride solution, dried (MgSO₄) and concentrated. The obtained colorless crystals were recrystallized from ethyl acetate-hexane to give [3-(2-{3-ethoxy-1-[5-(trifluoromethyl)-2-yridinyl]-1H-pyrazol-4-yl}ethoxy)-1-phenyl-1H-pyrazol-4-yl]acetic acid (544 mg, yield 82%). melting point: 135-137° C.

### Example 73

To a mixture of 2-{3-ethoxy-1-[5-(trifluoromethyl)-2-pyridinyl]-1H-pyrazol-4-yl}ethanol (400 mg), methyl 4-hydroxyphenylacetate (243 mg), tributylphosphine (662 μL) and tetrahydrofuran (66 ml) was added 1,1′-azodicarbonyldipiperidine (670 mg) at room temperature and the mixture was stirred overnight. The reaction solution was concentrated. The residue was subjected to silica gel column chromatography, and a white solid was obtained from a fraction eluted with ethyl acetate-hexane (1:4, volume ratio). A mixture of the obtained oily substance, 1N aqueous sodium hydroxide solution (30 ml), tetrahydrofuran (30 ml) and ethanol (30 ml) was stirred at room temperature for 3 hours. 1N Hydrochloric acid (30 ml) was added and the mixture was extracted with ethyl acetate. The ethyl acetate layer was washed with saturated aqueous sodium chloride solution, dried (MgSO₄) and concentrated. The obtained colorless crystals were recrystallized from ethyl acetate-hexane to give [4-(2-{3-ethoxy-1-[5-(trifluoromethyl)-2-pyridinyl]-1H-pyrazol-4-yl}ethoxy)phenyl]acetic acid (123 mg, yield 21%). melting point: 142-143° C.

### Example 74

To a mixture of 2-{3-ethoxy-1-[5-(trifluoromethyl)-2-pyridinyl]-1H-pyrazol-4-yl}ethanol (400 mg), methyl 2-(3-hydroxyphenoxy)-2-methylpropanoate (335 mg), tributylphosphine (662 μL) and tetrahydrofuran (66 ml) was added 1,1′-azodicarbonyldipiperidine (670 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (30 ml), tetrahydrofuran (30 ml) and ethanol (30 ml) was stirred at room temperature overnight. 1N Hydrochloric acid (30 ml) was added and the mixture was extracted with ethyl acetate. The ethyl acetate layer was washed with saturated aqueous sodium chloride solution, dried (MgSO₄) and concentrated. The residue was subjected to silica gel column chromatography, and colorless crystals were obtained from a fraction eluted with ethyl acetate-hexane (1:1, volume ratio). The obtained colorless crystals were recrystallized from ethyl acetate-hexane to give 2-[3-(2-{3-ethoxy-1-[5-(trifluoromethyl)-2-pyridinyl]-1H-pyrazol-4-yl}ethoxy)phenoxy]-2-methylpropanoic acid (169 mg, yield 26%). melting point: 89-90° C.

### Example 75

To a mixture of 2-{3-ethoxy-1-[5-(trifluoromethyl)-2-pyridinyl]-1H-pyrazol-4-yl}ethanol (350 mg), methyl 3-(4-hydroxy-2-methoxyphenyl)propanoate (733 mg), tributylphosphine (868 μL) and tetrahydrofuran (58 ml) was added 1,1′-azodicarbonyldipiperidine (879 mg) at room temperature and the mixture was stirred overnight. The reaction solution was concentrated. The residue was subjected to silica gel column chromatography, and a pale-yellow oily substance was obtained from a fraction eluted with ethyl acetate-hexane (1:3, volume ratio). A mixture of the obtained oily substance, 1N aqueous sodium hydroxide solution (30 ml), tetrahydrofuran (30 ml) and ethanol (30 ml) was stirred at room temperature for 3 days. 1N Hydrochloric acid (30 ml) was added and the mixture was extracted with ethyl acetate. The ethyl acetate layer was washed with saturated aqueous sodium chloride solution, dried (MgSO₄) and concentrated. The residue was subjected to silica gel column chromatography, and colorless crystals were obtained from a fraction eluted with ethyl acetate-hexane (1:1.5, volume ratio). The obtained colorless crystals were recrystallized from ethyl acetate-hexane to give 3-[4-(2-{3-ethoxy-1-[5-(trifluoromethyl)-2-pyridinyl]-1H-pyrazol-4-yl}ethoxy)-2-methoxyphenyl]propanoic acid (337 mg, yield 61%). melting point: 147-148° C.

### Example 76

To a mixture of 2-{3-ethoxy-1-[5-(trifluoromethyl)-2-pyridinyl]-1H-pyrazol-4-yl}ethanol (400 mg), ethyl 3-(4-hydroxy-2-methylphenyl)propanoate (332 mg), tributylphosphine (662 μL) and tetrahydrofuran (66 ml) was added 1,1′-azodicarbonyldipiperidine (670 mg) at room temperature and the mixture was stirred overnight. The reaction solution was concentrated. The residue was subjected to silica gel column chromatography, and a colorless oil was obtained from a fraction eluted with ethyl acetate-hexane (1:5, volume ratio). A mixture of the obtained oily substance, 1N aqueous sodium hydroxide solution (30 ml), tetrahydrofuran (30 ml) and ethanol (30 ml) was stirred at room temperature overnight. 1N Hydrochloric acid (30 ml) was added and the mixture was extracted with ethyl acetate. The ethyl acetate layer was washed with saturated aqueous sodium chloride solution, dried (MgSO₄) and concentrated. The obtained colorless crystals were recrystallized from ethyl acetate-hexane to give 3-[4-(2-{3-ethoxy-1-[5-(trifluoromethyl)-2-pyridinyl]-1H-pyrazol-4-yl}ethoxy)-2-methylphenyl]propanoic acid (210 mg, yield 34%). melting point: 117-119° C.

### Example 77

A mixture of 4-{3-propyl-1-[5-(trifluoromethyl)-2-pyridinyl]-1H-pyrazol-4-yl}butyl methanesulfonate (500 mg), sodium hydride (60%, in oil, 74.0 mg) and N,N-dimethylformamide (10 ml) was stirred at room temperature for 30 minutes and a solution of ethyl 3-[3-(4-fluorophenyl)-1H-pyrazol-4-yl]propanoate (350 mg) in N,N-dimethylformamide (2 ml) was added. After stirring overnight, water was added, and the mixture was extracted with ethyl acetate. The ethyl acetate layer was washed with saturated aqueous sodium chloride solution, dried (MgSO₄) and concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (30 ml), tetrahydrofuran (30 ml) and ethanol (30 ml) was stirred at room temperature overnight. 1N Hydrochloric acid (30 ml) was added and the mixture was extracted with ethyl acetate. The ethyl acetate layer was washed with saturated aqueous sodium chloride solution, dried (MgSO₄) and concentrated. The obtained colorless crystals were recrystallized from ethyl acetate-hexane to give 3-[3-(4-fluorophenyl)-1-(4-{3-propyl-1-[5-(trifluoromethyl)-2-pyridinyl]-1H-pyrazol-4-yl}butyl)-1H-pyrazol-4-yl]propanoic acid (395 mg, yield 59%). melting point: 119-121° C.

### Example 78

A mixture of ethyl 3-(3-ethoxy-1H-pyrazol-4-yl)propanoate (500 mg), sodium hydride (60%, in oil, 113 mg) and N,N-dimethylformamide (22 ml) was stirred at room temperature for 1 hour and 4-{3-propyl-1-[5-(trifluoromethyl)-2-pyridinyl]-1H-pyrazol-4-yl}butyl methanesulfonate (870 mg) was added. After stirring the resulting mixture overnight, 0.1N aqueous hydrochloric acid solution (100 ml) was added and the mixture was extracted with ethyl acetate. The ethyl acetate layer was washed with saturated aqueous sodium chloride solution, dried (MgSO₄) and concentrated. A mixture of the obtained oily substance, 1N aqueous sodium hydroxide solution (30 ml), tetrahydrofuran (30 ml) and ethanol (30 ml) was stirred at room temperature for 3 hours. 1N Hydrochloric acid (30 ml) was added and the mixture was extracted with ethyl acetate. The ethyl acetate layer was washed with saturated aqueous sodium chloride solution, dried (MgSO₄) and concentrated. The residue was subjected to silica gel column chromatography, and a colorless oil was obtained from a fraction eluted with ethyl acetate-hexane (1:1, volume ratio). A mixture of the obtained oily substance, 1N aqueous sodium hydroxide solution (1.39 ml), tetrahydrofuran (30 ml) and ethanol (30 ml) was stirred at room temperature for 1 hour and concentrated. The obtained colorless crystals were recrystallized from ethyl acetate-hexane to give sodium 3-[3-ethoxy-1-(4-{3-propyl-1-[5-(trifluoromethyl)-2-pyridinyl]-1H-pyrazol-4-yl}butyl)-1H-pyrazol-4-yl]propanoate (657 mg, yield 59%). melting point: 250-251° C.

### Example 79

To a mixture of 3-{3-propyl-1-[5-(trifluoromethyl)-2-pyridyl]-1H-pyrazol-4-yl}-1-propanol (470 mg), ethyl 4-hydroxy-3-methoxyphenylacetate (320 mg), tributylphosphine (610 mg) and tetrahydrofuran (30 ml) was added 1,1′-azodicarbonyldipiperidine (760 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours. 1N Hydrochloric acid (5 ml) was added and the mixture was extracted with ethyl acetate. The ethyl acetate layer was washed with saturated aqueous sodium chloride solution, dried (MgSO₄) and concentrated. The obtained colorless crystals were collected by filtration to give [3-methoxy-4-(3-{3-propyl-1-[5-(trifluoromethyl)-2-pyridyl]-1H-pyrazol-4-yl}propoxy)phenyl]acetic acid (550 mg, yield 77%). The crystals were recrystallized from ethyl acetate-hexane. melting point: 121-122° C.

### Example 80

To a mixture of 3-{3-propyl-1-[5-(trifluoromethyl)-2-pyridinyl]-1H-pyrazol-4-yl}-1-propanol (510 mg), methyl 3-hydroxy-4-methoxyphenylacetate (799 mg), tributylphosphine (1.01 ml) and tetrahydrofuran (100 ml) was added 1,1′-azodicarbonyldipiperidine (1.03 g) at room temperature and the mixture was stirred overnight. The reaction solution was concentrated. The residue was subjected to silica gel column chromatography, and a colorless oil was obtained from a fraction eluted with ethyl acetate-hexane (1:5, volume ratio). A mixture of the obtained oily substance, 1N aqueous sodium hydroxide solution (30 ml), tetrahydrofuran (30 ml) and ethanol (30 ml) was stirred at room temperature overnight. 1N Hydrochloric acid (30 ml) was added and the mixture was extracted with ethyl acetate. The ethyl acetate layer was washed with saturated aqueous sodium chloride solution, dried (MgSO₄) and concentrated. The obtained colorless crystals were recrystallized from ethyl acetate-hexane to give [4-methoxy-3-(3-{3-propyl-1-[5-(trifluoromethyl)-2-pyridinyl]-1H-pyrazol-4-yl}propoxy)phenyl]acetic acid (451 mg, yield 58%). melting point: 124-126° C.

### Example 81

To a mixture of 3-{3-propyl-1-[5-(trifluoromethyl)-2-pyridinyl]-1H-pyrazol-4-yl}-1-propanol (560 mg), ethyl 3-(5-hydroxy-2-methoxyphenyl)propanoate (441 mg), tributylphosphine (892 μL) and tetrahydrofuran (100 ml) was added 1,1′-azodicarbonyldipiperidine (903 mg) at room temperature and the mixture was stirred overnight. The reaction solution was concentrated. The residue was subjected to silica gel column chromatography, and a colorless oil was obtained from a fraction eluted with ethyl acetate-hexane (1:5, volume ratio). A mixture of the obtained oily substance, 1N aqueous sodium hydroxide solution (30 ml), tetrahydrofuran (30 ml) and ethanol (30 ml) was stirred at room temperature for 5 hours. 1N Hydrochloric acid (30 ml) was added and the mixture was extracted with ethyl acetate. The ethyl acetate layer was washed with saturated aqueous sodium chloride solution, dried (MgSO₄) and concentrated. The residue was subjected to silica gel column chromatography, and colorless crystals were obtained from a fraction eluted with ethyl acetate-hexane (1:1, volume ratio). The obtained colorless crystals were recrystallized from ethyl acetate-hexane to give 3-[2-methoxy-5-(3-{3-propyl-1-[5-(trifluoromethyl)-2-pyridinyl]-1H-pyrazol-4-yl}propoxy)phenyl]propanoic acid (407 mg, yield 46%). melting point: 104-106° C.

### Example 82

To a mixture of 3-{3-propyl-1-[5-(trifluoromethyl)-2-pyridyl]-1H-pyrazol-4-yl}-1-propanol (500 mg), methyl 3-(4-hydroxyphenyl)propionate (300 mg), tributylphosphine (700 mg) and tetrahydrofuran (30 ml) was added 1,1′-azodicarbonyldipiperidine (810 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours. 1N Hydrochloric acid (5 ml) was added and the mixture was extracted with ethyl acetate. The ethyl acetate layer was washed with saturated aqueous sodium chloride solution, dried (MgSO₄) and concentrated. The obtained colorless crystals were collected by filtration to give 3-[4-(3-{3-propyl-1-[5-(trifluoromethyl)-2-pyridyl]-1H-pyrazol-4-yl}propoxy)phenyl]propionic acid (650 mg, yield 88%). The crystals were recrystallized from ethyl acetate-hexane. melting point: 118-119° C.

### Example 83

To a mixture of 3-{3-propyl-1-[5-(trifluoromethyl)-2-pyridyl]-1H-pyrazol-4-yl}-1-propanol (500 mg), methyl 3-(2-hydroxyphenyl)propionate (300 mg), tributylphosphine (700 mg) and tetrahydrofuran (30 ml) was added 1,1′-azodicarbonyldipiperidine (800 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours. 1N Hydrochloric acid (5 ml) was added and the mixture was extracted with ethyl acetate. The ethyl acetate layer was washed with saturated aqueous sodium chloride solution, dried (MgSO₄) and concentrated. The obtained colorless crystals were collected by filtration to give 3-[2-(3-{3-propyl-1-[5-(trifluoromethyl)-2-pyridyl]-1H-pyrazol-4-yl}propoxy)phenyl]propionic acid (420 mg, yield 57%). The crystals were recrystallized from ethyl acetate-hexane. melting point: 87-88° C.

### Example 84

To a mixture of 3-{3-propyl-1-[5-(trifluoromethyl)-2-pyridyl]-1H-pyrazol-4-yl}-1-propanol (500 mg), methyl 3-(3-hydroxyphenyl)propionate (300 mg), tributylphosphine (700 mg) and tetrahydrofuran (30 ml) was added 1,1′-azodicarbonyldipiperidine (800 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours. 1N Hydrochloric acid (5 ml) was added and the mixture was extracted with ethyl acetate. The ethyl acetate layer was washed with saturated aqueous sodium chloride solution, dried (MgSO₄) and concentrated. The obtained colorless crystals were collected by filtration to give 3-[3-(3-{3-propyl-1-[5-(trifluoromethyl)-2-pyridyl]-1H-pyrazol-4-yl}propoxy)phenyl]propionic acid (520 mg, yield 71%). The crystals were recrystallized from ethyl acetate-hexane. melting point: 97-98° C.

### Example 85

To a mixture of 3-{(3-ethoxy-1-[5-(trifluoromethyl)-2-pyridyl]-1H-pyrazol-4-yl}-1-propanol (500 mg), methyl 3-(4-hydroxy-2-methoxyphenyl)propionate (340 mg), tributylphosphine (650 mg) and tetrahydrofuran (30 ml) was added 1,1′-azodicarbonyldipiperidine (810 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours. 1N Hydrochloric acid (5 ml) was added and the mixture was extracted with ethyl acetate. The ethyl acetate layer was washed with saturated aqueous sodium chloride solution, dried (MgSO₄) and concentrated. The obtained colorless crystals were collected by filtration to give 3-[4-(3-{3-ethoxy-1-[5-(trifluoromethyl)-2-pyridyl]-1H-pyrazol-4-yl}propoxy)-2-methoxyphenyl]propionic acid (530 mg, yield 67%). The crystals were recrystallized from ethyl acetate-hexane. melting point: 120-121° C.

### Example 86

To a mixture of 3-{3-propyl-1-[5-(trifluoromethyl)-2-pyridyl]-1H-pyrazol-4-yl}-1-propanol (510 mg), methyl 3-(4-hydroxy-2-methoxyphenyl)propionate (360 mg), tributylphosphine (650 mg) and tetrahydrofuran (30 ml) was added 1,1′-azodicarbonyldipiperidine (810 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours. 1N Hydrochloric acid (5 ml) was added and the mixture was extracted with ethyl acetate. The ethyl acetate layer was washed with saturated aqueous sodium chloride solution, dried (MgSO₄) and concentrated. The obtained colorless crystals were collected by filtration to give 3-[2-methoxy-4-(3-{3-propyl-1-[5-(trifluoromethyl)-2-pyridyl]-1H-pyrazol-4-yl}propoxy)phenyl]propionic acid (520 mg, yield 65%). The crystals were recrystallized from ethyl acetate-hexane. melting point: 114-115° C.

### Example 87

To a mixture of 3-{3-propyl-1-[5-(trifluoromethyl)-2-pyridyl]-1H-pyrazol-4-yl}-1-propanol (510 mg), methyl (3-hydroxy-1-methyl-1H-pyrazol-4-yl)acetate (290 mg), tributylphosphine (680 mg) and tetrahydrofuran (30 ml) was added 1,1′-azodicarbonyldipiperidine (860 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours. 1N Hydrochloric acid (5 ml) was added and the mixture was extracted with ethyl acetate. The ethyl acetate layer was washed with saturated aqueous sodium chloride solution, dried (MgSO₄) and concentrated. The obtained colorless crystals were collected by filtration to give [1-methyl-3-(3-{3-propyl-1-[5-(trifluoromethyl)-2-pyridyl]-1H-pyrazol-4-yl}propoxy)-1H-pyrazol-4-yl]acetic acid (570 mg, yield 77%). The crystals were recrystallized from ethyl acetate-hexane. melting point: 119-120° C.

### Example 88

To a mixture of 4-{3-propyl-1-[5-(trifluoromethyl)-2-pyridyl]-1H-pyrazol-4-yl}-1-butanol (500 mg), methyl 4-hydroxyphenylacetate (270 mg), tributylphosphine (620 mg) and tetrahydrofuran (30 ml) was added 1,1′-azodicarbonyldipiperidine (780 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours. 1N Hydrochloric acid (5 ml) was added and the mixture was extracted with ethyl acetate. The ethyl acetate layer was washed with saturated aqueous sodium chloride solution, dried (MgSO₄) and concentrated. The obtained colorless crystals were collected by filtration to give [4-(4-{3-propyl-1-[5-(trifluoromethyl)-2-pyridyl]-1H-pyrazol-4-yl}butoxy)phenyl]acetic acid (410 mg, yield 58%). The crystals were recrystallized from ethyl acetate-hexane. melting point: 121-122° C.

### Example 89

To a mixture of 4-{3-propyl-1-[5-(trifluoromethyl)-2-pyridyl]-1H-pyrazol-4-yl}-1-butanol (510 mg), methyl 3-(4-hydroxy-2-methoxyphenyl)propionate (330 mg), tributylphosphine (630 mg) and tetrahydrofuran (30 ml) was added 1,1′-azodicarbonyldipiperidine (790 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours. 1N Hydrochloric acid (5 ml) was added and the mixture was extracted with ethyl acetate. The ethyl acetate layer was washed with saturated aqueous sodium chloride solution, dried (MgSO₄) and concentrated. The obtained colorless crystals were collected by filtration to give 3-[2-methoxy-4-(4-{3-propyl-1-[5-(trifluoromethyl)-2-pyridyl]-1H-pyrazol-4-yl}butoxy)phenyl]propionic acid (510 mg, yield 65%). The crystals were recrystallized from ethyl acetate-hexane. melting point: 91-92° C.

### Example 90

To a mixture of 3-{3-propyl-1-[5-(trifluoromethyl)-2-pyridyl]-1H-pyrazol-4-yl}-1-propanol (190 mg), methyl 2-fluoro-5-hydroxyphenylacetate (110 mg), tributylphosphine (250 mg) and tetrahydrofuran (20 ml) was added 1,1′-azodicarbonyldipiperidine (310 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours. 1N Hydrochloric acid (5 ml) was added and the mixture was extracted with ethyl acetate. The ethyl acetate layer was washed with saturated aqueous sodium chloride solution, dried (MgSO₄) and concentrated. The obtained colorless crystals were collected by filtration to give [2-fluoro-5-(3-{3-propyl-1-[5-(trifluoromethyl)-2-pyridyl]-1H-pyrazol-4-yl}propoxy)phenyl]acetic acid (220 mg, yield 79%). The crystals were recrystallized from ethyl acetate-hexane. melting point: 111-112° C.

### Example 91

To a mixture of 3-{3-propyl-1-[5-(trifluoromethyl)-2-pyridyl]-1H-pyrazol-4-yl}-1-propanol (390 mg), methyl 4-fluoro-3-hydroxyphenylacetate (230 mg), tributylphosphine (510 mg) and tetrahydrofuran (30 ml) was added 1,1′-azodicarbonyldipiperidine (640 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours. 1N Hydrochloric acid (5 ml) was added and the mixture was extracted with ethyl acetate. The ethyl acetate layer was washed with saturated aqueous sodium chloride solution, dried (MgSO₄) and concentrated. The obtained colorless crystals were collected by filtration to give [4-fluoro-3-(3-{3-propyl-1-[5-(trifluoromethyl)-2-pyridyl]-1H-pyrazol-4-yl}propoxy)phenyl]acetic acid (220 mg, yield 79%). The crystals were recrystallized from ethyl acetate-hexane. melting point: 88-89° C.

### Example 92

To a mixture of 3-{3-propyl-1-[5-(trifluoromethyl)-2-pyridyl]-1H-pyrazol-4-yl}-1-propanol (500 mg), ethyl 3-(3-hydroxy-5-methoxyphenyl)propionate (380 mg), tributylphosphine (650 mg) and tetrahydrofuran (30 ml) was added 1,1′-azodicarbonyldipiperidine (650 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (2 ml), tetrahydrofuran (4 ml) and ethanol (4 ml) was stirred at room temperature for 5 hours. 1N Hydrochloric acid (2 ml) was added and the mixture was extracted with ethyl acetate. The ethyl acetate layer was washed with saturated aqueous sodium chloride solution, dried (MgSO₄) and concentrated. The obtained colorless crystals were collected by filtration to give 3-[3-methoxy-5-(3-{3-propyl-1-[5-(trifluoromethyl)-2-pyridyl]-1H-pyrazol-4-yl}propoxy)phenyl]propionic acid (380 mg, yield 48%). The crystals were recrystallized from isopropyl ether-hexane. melting point: 98-99° C.

### Example 93

To a mixture of 3-{3-propyl-1-[5-(trifluoromethyl)-2-pyridyl]-1H-pyrazol-4-yl}-1-propanol (500 mg), ethyl 3-(3-hydroxy-4-methoxyphenyl)propionate (360 mg), tributylphosphine (650 mg) and tetrahydrofuran (35 ml) was added 1,1′-azodicarbonyldipiperidine (810 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and ethanol (5 ml) was stirred at room temperature for 5 hours. 1N Hydrochloric acid (5 ml) was added and the mixture was extracted with ethyl acetate. The ethyl acetate layer was washed with saturated aqueous sodium chloride solution, dried (MgSO₄) and concentrated. The obtained colorless crystals were collected by filtration to give 3-[4-methoxy-3-(3-{3-propyl-1-[5-(trifluoromethyl)-2-pyridyl]-1H-pyrazol-4-yl}propoxy)phenyl]propionic acid (280 mg, yield 70%). melting point: 147-148° C.

### Example 94

To a mixture of 3-{3-propyl-1-[5-(trifluoromethyl)-2-pyridyl]-1H-pyrazol-4-yl}-1-propanol (650 mg), methyl 4-hydroxy-2-methylphenylacetate (390 mg), tributylphosphine (840 mg) and tetrahydrofuran (30 ml) was added 1,1′-azodicarbonyldipiperidine (1050 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours. 1N Hydrochloric acid (5 ml) was added and the mixture was extracted with ethyl acetate. The ethyl acetate layer was washed with saturated aqueous sodium chloride solution, dried (MgSO₄) and concentrated. The obtained colorless crystals were collected by filtration to give [2-methyl-4-(3-{3-propyl-1-[5-(trifluoromethyl)-2-pyridyl]-1H-pyrazol-4-yl}propoxy)phenyl]acetic acid (590 mg, yield 62%). The crystals were recrystallized from ethyl acetate-hexane. melting point: 134-135° C.

### Example 95

To a mixture of 3-{3-propyl-1-[5-(trifluoromethyl)-2-pyridyl]-1H-pyrazol-4-yl}-1-propanol (470 mg), methyl 4-hydroxy-2-methoxyphenylacetate (300 mg), tributylphosphine (610 mg) and tetrahydrofuran (30 ml) was added 1,1′-azodicarbonyldipiperidine (760 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours. 1N Hydrochloric acid (5 ml) was added and the mixture was extracted with ethyl acetate. The ethyl acetate layer was washed with saturated aqueous sodium chloride solution, dried (MgSO₄) and concentrated. The obtained colorless crystals were collected by filtration to give [2-methoxy-4-(3-{3-propyl-1-[5-(trifluoromethyl)-2-pyridyl]-1H-pyrazol-4-yl}propoxy)phenyl]acetic acid (580 mg, yield 81%). The crystals were recrystallized from ethyl acetate-hexane. melting point: 135-136° C.

### Example 96

To a mixture of 3-{3-propyl-1-[5-(trifluoromethyl)-2-pyridyl]-1H-pyrazol-4-yl}-1-propanol (470 mg), ethyl 3-(4-hydroxy-3-methoxyphenyl)propionate (350 mg), tributylphosphine (610 mg) and tetrahydrofuran (30 ml) was added 1,1′-azodicarbonyldipiperidine (760 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give 3-[3-methoxy-4-(3-{3-propyl-1-[5-(trifluoromethyl)-2-pyridyl]-1H-pyrazol-4-yl}propoxy)phenyl]propionic acid (590 mg, yield 80%). The crystals were recrystallized from ethyl acetate-hexane. melting point: 126-127° C.

### Example 97

To a mixture of 3-{3-propyl-1-[5-(trifluoromethyl)-2-pyridinyl]-1H-pyrazol-4-yl}-1-propanol (580 mg), methyl (5-hydroxy-2-methoxyphenyl)acetate (400 mg), tributylphosphine (924 μL) and tetrahydrofuran (90 ml) was added 1,1′-azodicarbonyldipiperidine (936 mg) at room temperature and the mixture was stirred for 3 days. The reaction solution was concentrated. The residue was subjected to silica gel column chromatography, and a pale-yellow oily substance was obtained from a fraction eluted with ethyl acetate-hexane (1:6, volume ratio). A mixture of the obtained oily substance, 1N aqueous sodium hydroxide solution (30 ml), tetrahydrofuran (30 ml) and ethanol (30 ml) was stirred at room temperature overnight. 1N Hydrochloric acid (30 ml) was added and the mixture was extracted with ethyl acetate. The ethyl acetate layer was washed with saturated aqueous sodium chloride solution, dried (MgSO₄) and concentrated. The obtained pale-yellow crystals were recrystallized from ethyl acetate-hexane to give [2-methoxy-5-(3-{3-propyl-1-[5-(trifluoromethyl)-2-pyridinyl]-1H-pyrazol-4-yl}propoxy)phenyl]acetic acid (483 mg, yield 55%) as colorless crystals. melting point: 135-136° C.

### Example 98

To a mixture of 3-{3-propyl-1-[5-(trifluoromethyl)-2-pyridinyl]-1H-pyrazol-4-yl}-1-propanol (500 mg), methyl 3-(4-hydroxy-2-ethoxyphenyl)propanoate (395 mg), tributylphosphine (797 μL) and tetrahydrofuran (80 ml) was added 1,1′-azodicarbonyldipiperidine (807 mg) at room temperature and the mixture was stirred overnight. The reaction solution was concentrated. The residue was subjected to silica gel column chromatography, and a white solid was obtained from a fraction eluted with ethyl acetate-hexane (1:7, volume ratio). A mixture of the obtained solid, 1N aqueous sodium hydroxide solution (30 ml), tetrahydrofuran (30 ml) and ethanol (30 ml) was stirred overnight at room temperature. 1N Hydrochloric acid (30 ml) was added and the mixture was extracted with ethyl acetate. The ethyl acetate layer was washed with saturated aqueous sodium chloride solution, dried (MgSO₄) and concentrated. The obtained white solid was recrystallized from ethyl acetate-hexane to give 3-[2-ethoxy-4-(3-{3-propyl-1-[5-(trifluoromethyl)-2-pyridinyl]-1H-pyrazol-4-yl}propoxy)phenyl]propanoic acid (442 mg, yield 55%) as colorless crystals. melting point: 119-120° C.

### Example 99

To a mixture of 3-{3-phenyl-1-[5-(trifluoromethyl)-2-pyridinyl]-1H-pyrazol-4-yl}-1-propanol (400 mg), methyl 3-(4-hydroxy-2-ethoxyphenyl)propanoate (260 mg), tributylphosphine (480 μL) and tetrahydrofuran (30 ml) was added 1,1′-azodicarbonyldipiperidine (600 mg) at room temperature and the mixture was stirred overnight. The reaction solution was concentrated. The residue was subjected to silica gel column chromatography, and a white solid was obtained from a fraction eluted with ethyl acetate-hexane (1:7, volume ratio). A mixture of the obtained solid, 1N aqueous sodium hydroxide solution (30 ml), tetrahydrofuran (30 ml) and ethanol (30 ml) was stirred overnight at room temperature. 1N Hydrochloric acid (30 ml) was added and the mixture was extracted with ethyl acetate. The ethyl acetate layer was washed with saturated aqueous sodium chloride solution, dried (MgSO₄) and concentrated. The obtained white solid was recrystallized from ethyl acetate-hexane to give 3-[2-ethoxy-4-(3-{3-phenyl-1-[5-(trifluoromethyl)-2-pyridinyl]-1H-pyrazol-4-yl}propoxy)phenyl]propanoic acid (373 mg, yield 60%) as colorless crystals. melting point: 135-136° C.

### Example 100

A mixture of ethyl 2-{3-[3-(3-ethoxy-1H-pyrazol-4-yl)propoxy]phenoxy}-2-methylpropanoate (300 mg), sodium hydride (60%, in oil, 63.6 mg) and N,N-dimethylformamide (10 ml) was stirred at room temperature for 30 minutes and iodocyclopentane (184 μL) was added. After stirring overnight, saturated aqueous ammonium chloride solution was added, and the mixture was extracted with ethyl acetate. The ethyl acetate layer was washed with saturated aqueous sodium chloride solution, dried (MgSO₄) and concentrated. A mixture of the obtained residue, 1N aqueous sodium hydroxide solution (25 ml), tetrahydrofuran (25 ml) and ethanol (25 ml) was stirred overnight at room temperature. 1N Hydrochloric acid (25 ml) was added and the mixture was extracted with ethyl acetate. The ethyl acetate layer was washed with saturated aqueous sodium chloride solution, dried (MgSO₄) and concentrated. The residue was subjected to silica gel column chromatography, and a colorless oil was obtained from a fraction eluted with ethyl acetate-hexane (1:1, volume ratio). A mixture of the obtained oily substance, 1N aqueous sodium hydroxide solution (645 μL), tetrahydrofuran (25 ml) and ethanol (25 ml) was stirred at room temperature for 1 hour and concentrated. To a mixture of the obtained residue and water (25 ml) was added calcium chloride (69.0 mg) dissolved in a small amount of water, and the mixture was stirred overnight at room temperature. The resulting white precipitates were collected by filtration to give calcium 2-{3-[3-(1-cyclopentyl-3-ethoxy-1H-pyrazol-4-yl)propoxy]phenoxy}-2-methylpropanoate (256 mg, yield 74%) as amorphous.

¹H-NMR (DMSO-d₆) δ: 1.25 (3H, t, J=6.9 Hz), 1.41 (6H, s), 1.52-1.61 (2H, m), 1.67-2.00 (8H, m), 2.32-2.39 (2H, m), 3.83-3.90 (2H, m), 4.09 (2H, q, J=6.9 Hz), 4.34-4.45 (1H, m), 6.34-6.44 (3H, m), 6.96-7.04 (1H, m), 7.35 (1H,

### Example 101

To a mixture of 3-[3-ethoxy-1-(2-pyridinyl)-1H-pyrazol-4-yl]-1-propanol (420 mg), ethyl 2-(3-hydroxyphenoxy)-2-methylpropanoate (419 mg), tributylphosphine (847 μL) and tetrahydrofuran (34 ml) was added 1,1′-azodicarbonyldipiperidine (858 mg) at room temperature and the mixture was stirred overnight. The reaction solution was concentrated. The residue was subjected to silica gel column chromatography, and a colorless oil was obtained from a fraction eluted with ethyl acetate-hexane (1:6, volume ratio). A mixture of the obtained oily substance, 1N aqueous sodium hydroxide solution (25 ml), tetrahydrofuran (25 ml) and ethanol (25 ml) was stirred overnight at room temperature. 1N Hydrochloric acid (25 ml) was added and the mixture was extracted with ethyl acetate. The ethyl acetate layer was washed with saturated aqueous sodium chloride solution, dried (MgSO₄) and concentrated. The obtained colorless crystals were recrystallized from ethyl acetate-hexane to give 2-(3-{3-[3-ethoxy-1-(2-pyridinyl)-1H-pyrazol-4-yl]propoxy}phenoxy)-2-methylpropanoic acid (291 mg, yield 40%). melting point: 99-101° C.

### Example 102

A mixture of ethyl 2-{3-[4-(3-ethoxy-1H-pyrazol-4-yl)butoxy]phenoxy}-2-methylpropanoate (740 mg), sodium hydride (60%, in oil, 90.8 mg) and N,N-dimethylformamide (20 ml) was stirred at room temperature for 30 minutes and 2-chloro-5-(trifluoromethyl)pyridine (412 mg) was added. After stirring the reaction mixture overnight, saturated aqueous ammonium chloride solution was added, and the mixture was extracted with ethyl acetate. The ethyl acetate layer was washed with saturated aqueous sodium chloride solution, dried (MgSO₄) and concentrated. The residue was subjected to silica gel column chromatography, and a colorless oil was obtained from a fraction eluted with ethyl acetate-hexane (1:6, volume ratio). A mixture of the obtained oily substance, 1N aqueous sodium hydroxide solution (25 ml), tetrahydrofuran (25 ml) and ethanol (25 ml) was stirred at room temperature for 2.5 days. 1N Hydrochloric acid (25 ml) was added and the mixture was extracted with ethyl acetate. The ethyl acetate layer was washed with saturated aqueous sodium chloride solution, dried (MgSO₄) and concentrated. The residue was subjected to silica gel column chromatography, and a colorless oil was obtained from a fraction eluted with ethyl acetate-hexane (1:1, volume ratio). A mixture of the obtained oily substance, 1N aqueous sodium hydroxide solution (893 μL), tetrahydrofuran (25 ml) and ethanol (25 ml) was stirred at room temperature for 1 hour and concentrated. To a mixture of the obtained residue and water (25 ml) was added calcium chloride (90.8 mg) dissolved in a small amount of water and the mixture was stirred overnight at room temperature. The resulting white precipitates were collected by filtration to give calcium 2-[3-(4-{3-ethoxy-1-[5-(trifluoromethyl)-2-pyridinyl]-1H-pyrazol-4-yl}butoxy)phenoxy]-2-methylpropanoate (394 mg, yield 39%) as amorphous.

¹H-NMR (DMSO-d₆) δ: 1.36 (3H, t, J=7.2 Hz), 1.40 (6H, s), 1.62-1.78 (4H, m), 2.36-2.46 (2H, m), 3.85-3.94 (2H, m), 4.31 (2H, q, J=7.2 Hz), 6.34-6.44 (3H, m), 6.95-7.04 (1H, m), 7.79 (1H, d, J=8.7 Hz), 8.20-8.27 (1H, m), 8.32 (1H, s), 8.69-8.74 (1H, m).

### Example 103

To a mixture of 3-[3-ethoxy-1-(2-pyridinyl)-1H-pyrazol-4-yl]-1-propanol (300 mg), ethyl 3-(3-hydroxy-1-phenyl-1H-pyrazol-5-yl)propanoate (346 mg), tributylphosphine (603 μL) and tetrahydrofuran (25 ml) was added 1,1′-azodicarbonyldipiperidine (611 mg) at room temperature and the mixture was stirred overnight. The reaction solution was concentrated. The residue was subjected to silica gel column chromatography, and a yellow oily substance was obtained from a fraction eluted with ethyl acetate-hexane (1:6, volume ratio). A mixture of the obtained oily substance, 1N aqueous sodium hydroxide solution (25 ml), tetrahydrofuran (25 ml) and ethanol (25 ml) was stirred overnight at room temperature. 1N Hydrochloric acid (25 ml) was added and the mixture was extracted with ethyl acetate. The ethyl acetate layer was washed with saturated aqueous sodium chloride solution, dried (MgSO₄) and concentrated. The obtained colorless crystals were recrystallized from ethyl acetate-hexane to give 3-(3-{3-[3-ethoxy-1-(2-pyridinyl)-1H-pyrazol-4-yl]propoxy}-1-phenyl-1H-pyrazol-5-yl)propanoic acid (483 mg, yield 87%). melting point: 156-157° C.

### Example 104

A mixture of ethyl 3-{3-[4-(3-ethoxy-1H-pyrazol-4-yl)butoxy]-1-phenyl-1H-pyrazol-5-yl}propanoate (900 mg), sodium hydride (60%, in oil, 101 mg) and N,N-dimethylformamide (20 ml) was stirred at room temperature for 30 minutes. 2-Chloro-5-(trifluoromethyl)pyridine (459 mg) was added. After stirring the obtained mixture overnight, saturated aqueous ammonium chloride solution was added, and the mixture was extracted with ethyl acetate. The ethyl acetate layer was washed with saturated aqueous sodium chloride solution, dried (MgSO₄) and concentrated. A mixture of the obtained yellow oily substance, 1N aqueous sodium hydroxide solution (25 ml), tetrahydrofuran (25 ml) and ethanol (25 ml) was stirred overnight at room temperature. 1N Hydrochloric acid (25 ml) was added and the mixture was extracted with ethyl acetate. The ethyl acetate layer was washed with saturated aqueous sodium chloride solution, dried (MgSO₄) and concentrated. The residue was subjected to silica gel column chromatography, and a white solid was obtained from a fraction eluted with ethyl acetate-hexane (1:2, volume ratio). The obtained solid was recrystallized from ethyl acetate-hexane to give 3-[3-(4-{3-ethoxy-1-[5-(trifluoromethyl)-2-pyridinyl]-1H-pyrazol-4-yl}butoxy)-1-phenyl-1H-pyrazol-5-yl]propanoic acid (640 mg, yield 56%) as colorless crystals. melting point: 138-139° C.

### Example 105

To a mixture of 3-{3-ethoxy-1-[4-(trifluoromethyl)phenyl]-1H-pyrazol-4-yl}-1-propanol (512 mg), ethyl 2-(3-hydroxyphenoxy)-2-methylpropanoate (401 mg), tributylphosphine (812 μL) and tetrahydrofuran (35 ml) was added 1,1′-azodicarbonyldipiperidine (823 mg) at room temperature and the mixture was stirred overnight. The reaction solution was concentrated. The residue was subjected to silica gel column chromatography, and a colorless oil was obtained from a fraction eluted with ethyl acetate-hexane (1:6, volume ratio). A mixture of the obtained oily substance, 1N aqueous sodium hydroxide solution (25 ml), tetrahydrofuran (25 ml) and ethanol (25 ml) was stirred overnight at room temperature. 1N Hydrochloric acid (25 ml) was added and the mixture was extracted with ethyl acetate. The ethyl acetate layer was washed with saturated aqueous sodium chloride solution, dried (MgSO₄) and concentrated. The residue was subjected to silica gel column chromatography, and a colorless oil was obtained from a fraction eluted with ethyl acetate-hexane (1:1, volume ratio). A mixture of the obtained oily substance, 1N aqueous sodium hydroxide solution (995 μL), tetrahydrofuran (25 ml) and ethanol (25 ml) was stirred at room temperature for 1 hour and concentrated. To a mixture of the obtained residue and water (50 ml) was added calcium chloride (110 mg) dissolved in water (5 ml) and the mixture was stirred overnight at room temperature. The resulting white precipitates were collected by filtration to give calcium 2-[3-(3-{3-ethoxy-1-[4-(trifluoromethyl)phenyl]-1H-pyrazol-4-yl}propoxy)phenoxy]-2-methylpropanoate (440 mg, yield 53%) as amorphous.

¹H-NMR (DMSO-d₆) δ: 1.28-1.48 (3H, m), 1.41 (6H, s), 1.87-2.08 (2H, m), 2.41-2.56 (2H, m), 3.86-4.00 (2H, m), 4.20-4.39 (2H, m), 6.31-6.52 (3H, m), 6.93-7.10 (1H, m), 7.69-7.96 (4H, m), 8.38 (1H, s).

### Example 106

To a mixture of 3-[3-ethoxy-1-(2-pyridinyl)-1H-pyrazol-4-yl]-1-propanol (300 mg), methyl 3-hydroxyphenylacetate (402 mg), tributylphosphine (603 μL) and tetrahydrofuran (25 ml) was added 1,1′-azodicarbonyldipiperidine (611 mg) at room temperature and the mixture was stirred for 3 days. The reaction solution was concentrated. The residue was subjected to silica gel column chromatography, and a colorless oil was obtained from a fraction eluted with ethyl acetate-hexane (1:6, volume ratio). A mixture of the obtained oily substance, 1N aqueous sodium hydroxide solution (25 ml), tetrahydrofuran (25 ml) and ethanol (25 ml) was stirred overnight at room temperature. 1N Hydrochloric acid (25 ml) was added and the mixture was extracted with ethyl acetate. The ethyl acetate layer was washed with saturated aqueous sodium chloride solution, dried (MgSO₄) and concentrated. The obtained colorless oil was recrystallized from diisopropyl ether-hexane to give (3-{3-[3-ethoxy-1-(2-pyridinyl)-1H-pyrazol-4-yl]propoxy}phenyl)acetic acid (247 mg, yield 53%) as colorless crystals. melting point: 66-67° C.

### Example 107

A mixture of methyl 3-{2-ethoxy-4-[4-(3-ethoxy-1H-pyrazol-4-yl)butoxy]phenyl}propanoate (860 mg), sodium hydride (60%, in oil, 106 mg) and N,N-dimethylformamide (25 ml) was stirred at room temperature for 30 minutes and 2-chloro-5-(trifluoromethyl)pyridine (479 mg) was added. After stirring overnight, saturated aqueous ammonium chloride solution was added, and the mixture was extracted with ethyl acetate. The ethyl acetate layer was washed with saturated aqueous sodium chloride solution, dried (MgSO₄) and concentrated. A mixture of the obtained yellow oily substance, 1N aqueous sodium hydroxide solution (25 ml), tetrahydrofuran (25 ml) and ethanol (25 ml) was stirred overnight at room temperature. 1N Hydrochloric acid (25 ml) was added and the mixture was extracted with ethyl acetate. The ethyl acetate layer was washed with saturated aqueous sodium chloride solution, dried (MgSO₄) and concentrated. The residue was subjected to silica gel column chromatography, and a white solid was obtained from a fraction eluted with ethyl acetate-hexane (1:2, volume ratio). The obtained solid was recrystallized from ethyl acetate-hexane to give 3-[2-ethoxy-4-(4-{3-ethoxy-1-[5-(trifluoromethyl)-2-pyridinyl]-1H-pyrazol-4-yl}butoxy)phenyl]propanoic acid (718 mg, yield 63%) as colorless crystals. melting point: 101-102° C.

### Example 108

To a mixture of 3-[3-ethoxy-1-(2-pyridinyl)-1H-pyrazol-4-yl]-1-propanol (300 mg), methyl 3-(2-ethoxy-4-hydroxyphenyl)propanoate (298 mg), tributylphosphine (603 μL) and tetrahydrofuran (25 ml) was added 1,1′-azodicarbonyldipiperidine (611 mg) at room temperature and the mixture was stirred overnight. The reaction solution was concentrated. The residue was subjected to silica gel column chromatography, and a colorless oil was obtained from a fraction eluted with ethyl acetate-hexane (1:6, volume ratio). A mixture of the obtained oily substance, 1N aqueous sodium hydroxide solution (25 ml), tetrahydrofuran (25 ml) and ethanol (25 ml) was stirred overnight at room temperature. 1N Hydrochloric acid (25 ml) was added and the mixture was extracted with ethyl acetate. The ethyl acetate layer was washed with saturated aqueous sodium chloride solution, dried (MgSO₄) and concentrated. The obtained colorless crystals were recrystallized from ethyl acetate-hexane to give 3-(2-ethoxy-4-{3-[3-ethoxy-1-(2-pyridinyl)-1H-pyrazol-4-yl]propoxy}phenyl)propanoic acid (323 mg, yield 61%). melting point: 110-111° C.

### Example 109

To a mixture of 3-[3-ethoxy-1-(2-pyridinyl)-1H-pyrazol-4-yl]-1-propanol (300 mg), ethyl 3-(4-hydroxy-3-methoxyphenyl)propanoate (298 mg), tributylphosphine (603 μL) and tetrahydrofuran (25 ml) was added 1,1′-azodicarbonyldipiperidine (611 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (25 ml), tetrahydrofuran (25 ml) and ethanol (25 ml) was stirred at room temperature for 5 hours. 1N Hydrochloric acid (25 ml) was added and the mixture was extracted with ethyl acetate. The ethyl acetate layer was washed with saturated aqueous sodium chloride solution, dried (MgSO₄) and concentrated. The obtained colorless crystals were recrystallized from ethyl acetate-hexane to give 3-(4-{3-[3-ethoxy-1-(2-pyridinyl)-1H-pyrazol-4-yl]propoxy}-3-methoxyphenyl)propanoic acid (416 mg, yield 81%). melting point: 92-93° C.

### Example 110

A mixture of ethyl 3-{(3-ethoxy-1-[4-(3-ethoxy-1H-pyrazol-4-yl)butyl]-1H-pyrazol-4-yl}propanoate (680 mg), sodium hydride (60%, in oil, 86.4 mg) and N,N-dimethylformamide (20 ml) was stirred at room temperature for 30 minutes and 2-chloro-5-(trifluoromethyl)pyridine (391 mg) was added. After stirring the obtained mixture for 7 hours, saturated aqueous ammonium chloride solution was added, and the mixture was extracted with ethyl acetate. The ethyl acetate layer was washed with saturated aqueous sodium chloride solution, dried (MgSO₄) and concentrated. A mixture of the obtained yellow oily substance, 1N aqueous sodium hydroxide solution (25 ml), tetrahydrofuran (25 ml) and ethanol (25 ml) was stirred at room temperature for 5 hours. 1N Hydrochloric acid (25 ml) was added and the mixture was extracted with ethyl acetate. The ethyl acetate layer was washed with saturated aqueous sodium chloride solution, dried (MgSO₄) and concentrated. The residue was subjected to silica gel column chromatography, and a colorless oil was obtained from a fraction eluted with ethyl acetate-hexane (1:1, volume ratio). A mixture of the obtained oily substance, 1N aqueous sodium hydroxide solution (1.25 ml), tetrahydrofuran (25 ml) and ethanol (25 ml) was stirred at room temperature for 1 hour, and concentrated. To a mixture of the obtained residue and water (50 ml) was added calcium chloride (134 mg) dissolved in a small amount of water and the mixture was stirred overnight at room temperature. The resulting white precipitates were collected by filtration to give calcium 3-[3-ethoxy-1-(4-{3-ethoxy-1-[5-(trifluoromethyl)-2-pyridinyl]-1H-pyrazol-4-yl}butyl)-1H-pyrazol-4-yl]propanoate (654 mg, yield 71%) as amorphous.

¹H-NMR (DMSO-d₆) δ: 1.22 (3H, t, J=6.9 Hz), 1.36 (3H, t, J=6.9 Hz), 1.40-1.54 (2H, m), 1.66-1.78 (2H, m), 2.26-2.44 (4H, m), 2.46-2.58 (2H, m), 3.69-3.78 (2H, m), 4.11 (2H, q, J=6.9 Hz), 4.27 (2H, q, J=6.9 Hz), 6.93 (1H, s), 7.71 (1H, d, J=8.4 Hz), 7.79-7.85 (1H, m), 8.05 (1H, s), 8.44-8.49 (1H, m).

### Example 111

To a mixture of 3-{3-ethoxy-1-[4-(trifluoromethyl)phenyl]-1H-pyrazol-4-yl}-1-propanol (157 mg), ethyl 3-(3-hydroxy-1-phenyl-1H-pyrazol-5-yl)propanoate (130 mg), tributylphosphine (249 μL) and tetrahydrofuran (20 ml) was added 1,1′-azodicarbonyldipiperidine (252 mg) at room temperature and the mixture was stirred overnight. The reaction solution was concentrated. The residue was subjected to silica gel column chromatography, and a colorless oil was obtained from a fraction eluted with ethyl acetate-hexane (1:6, volume ratio). A mixture of the obtained oily substance, 1N aqueous sodium hydroxide solution (25 ml), tetrahydrofuran (25 ml) and ethanol (25 ml) was stirred overnight at room temperature. 1N Hydrochloric acid (25 ml) was added and the mixture was extracted with ethyl acetate. The ethyl acetate layer was washed with saturated aqueous sodium chloride solution, dried (MgSO₄) and concentrated. The obtained colorless oil was recrystallized from ethyl acetate-hexane to give 3-[3-(3-{3-ethoxy-1-[4-(trifluoromethyl)phenyl]-1H-pyrazol-4-yl}propoxy)-1-phenyl-1H-pyrazol-5-yl]propanoic acid (99.5 mg, yield 38%) as colorless crystals. melting point: 126-127° C.

### Example 112

A mixture of ethyl 3-[2-(benzyloxy)-4-(3-{3-ethoxy-1-[5-(trifluoromethyl)-2-pyridinyl]-1H-pyrazol-4-yl}propoxy)phenyl]propanoate (250 mg), 1N aqueous sodium hydroxide solution (25 ml), tetrahydrofuran (25 ml) and ethanol (25 ml) was stirred overnight at room temperature. 1N Hydrochloric acid (25 ml) was added and the mixture was extracted with ethyl acetate. The ethyl acetate layer was washed with saturated aqueous sodium chloride solution, dried (MgSO₄) and concentrated. The obtained white solid was recrystallized from ethyl acetate-hexane to give 3-[2-(benzyloxy)-4-(3-{3-ethoxy-1-[5-(trifluoromethyl)-2-pyridinyl]-1H-pyrazol-4-yl}propoxy)phenyl]propanoic acid (237 mg, yield 99%) as colorless crystals. melting point: 128-130° C.

### Example 113

To a mixture of 3-{3-ethoxy-1-[4-(trifluoromethyl)phenyl]-1H-pyrazol-4-yl)-1-propanol (400 mg), methyl 3-hydroxyphenylacetate (422 mg), tributylphosphine (633 μL) and tetrahydrofuran (25 ml) was added 1,1′-azodicarbonyldipiperidine (641 mg) at room temperature and the mixture was stirred overnight. The reaction solution was concentrated. The residue was subjected to silica gel column chromatography, and a colorless oil was obtained from a fraction eluted with ethyl acetate-hexane (1:6, volume ratio). A mixture of the obtained oily substance, 1N aqueous sodium hydroxide solution (25 ml), tetrahydrofuran (25 ml) and ethanol (25 ml) was stirred at room temperature for 5 hours. 1N Hydrochloric acid (25 ml) was added and the mixture was extracted with ethyl acetate. The ethyl acetate layer was washed with saturated aqueous sodium chloride solution, dried (MgSO₄) and concentrated. The obtained colorless crystals were recrystallized from ethyl acetate-hexane to give [3-(3-{3-ethoxy-1-[4-(trifluoromethyl)phenyl]-1H-pyrazol-4-yl}propoxy)phenyl]acetic acid (424 mg, yield 74%). melting point: 108-109° C.

### Example 114

To a mixture of 3-{3-ethoxy-1-[4-(trifluoromethyl)phenyl]-1H-pyrazol-4-yl}-1-propanol (400 mg), methyl 3-(2-ethoxy-4-hydroxyphenyl)propanoate (314 mg), tributylphosphine (633 μL) and tetrahydrofuran (25 ml) was added 1,1′-azodicarbonyldipiperidine (641 mg) at room temperature and the mixture was stirred overnight. The reaction solution was concentrated. The residue was subjected to silica gel column chromatography, and a white solid was obtained from a fraction eluted with ethyl acetate-hexane (1:6, volume ratio). A mixture of the obtained solid, 1N aqueous sodium hydroxide solution (25 ml), tetrahydrofuran (25 ml) and ethanol (25 ml) was stirred at room temperature for 5 hours. 1N Hydrochloric acid (25 ml) was added and the mixture was extracted with ethyl acetate. The ethyl acetate layer was washed with saturated aqueous sodium chloride solution, dried (MgSO₄) and concentrated. The obtained white solid was recrystallized from ethyl acetate-hexane to give 3-[2-ethoxy-4-(3-{3-ethoxy-1-[4-(trifluoromethyl)phenyl]-1H-pyrazol-4-yl}propoxy)phenyl]propanoic acid (420 mg, yield 65%) as colorless crystals. melting point: 131-132° C.

### Example 115

To a mixture of 3-(3-ethoxy-1-[4-(trifluoromethyl)phenyl]-1H-pyrazol-4-yl}-1-propanol (400 mg), ethyl 3-(4-hydroxy-3-methoxyphenyl)propanoate (314 mg), tributylphosphine (633 μL) and tetrahydrofuran (25 ml) was added 1,1′-azodicarbonyldipiperidine (641 mg) at room temperature and the mixture was stirred overnight. The reaction solution was concentrated. The residue was subjected to silica gel column chromatography, and a colorless oil was obtained from a fraction eluted with ethyl acetate-hexane (1:6, volume ratio). A mixture of the obtained oily substance, 1N aqueous sodium hydroxide solution (25 ml), tetrahydrofuran (25 ml) and ethanol (25 ml) was stirred at room temperature for 5 hours. 1N Hydrochloric acid (25 ml) was added and the mixture was extracted with ethyl acetate. The ethyl acetate layer was washed with saturated aqueous sodium chloride solution, dried (MgSO₄) and concentrated. The obtained colorless oil was recrystallized from ethyl acetate-hexane to give 3-[4-(3-{3-ethoxy-1-[4-(trifluoromethyl)phenyl]-1H-pyrazol-4-yl}propoxy)-3-methoxyphenyl]propanoic acid (423 mg, yield 68%) as colorless crystals. melting point: 125-126° C.

### Example 116

To a mixture of ethyl 3-[4-(3-{3-ethoxy-1-[5-(trifluoromethyl)-2-pyridinyl]-1H-pyrazol-4-yl}propoxy)-2-hydroxyphenyl]propanoate (300 mg), isopropanol (49.5 μL), tributylphosphine (294 μL) and tetrahydrofuran (15 ml) was added 1,1′-azodicarbonyldipiperidine (298 mg) at room temperature and the mixture was stirred overnight. The reaction solution was concentrated. The residue was subjected to silica gel column chromatography, and a colorless oil was obtained from a fraction eluted with ethyl acetate-hexane (1:6, volume ratio). A mixture of the obtained oily substance, 1N aqueous sodium hydroxide solution (25 ml), tetrahydrofuran (25 ml) and ethanol (25 ml) was stirred overnight at room temperature. 1N Hydrochloric acid (25 ml) was added and the mixture was extracted with ethyl acetate. The ethyl acetate layer was washed with saturated aqueous sodium chloride solution, dried (MgSO₄) and concentrated. The obtained white solid was recrystallized from ethyl acetate-hexane to give 3-[4-(3-{3-ethoxy-1-[5-(trifluoromethyl)-2-pyridinyl]-1H-pyrazol-4-yl}propoxy)-2-isopropoxyphenyl]propanoic acid (76.0 mg, yield 25%) as colorless crystals. melting point: 104-105° C.

### Example 117

To a mixture of ethyl 3-[4-(3-{3-ethoxy-1-[5-(trifluoromethyl)-2-pyridinyl]-1H-pyrazol-4-yl}propoxy)-2-hydroxyphenyl]propanoate (400 mg), propanol (119 μL), tributylphosphine (393 μL) and tetrahydrofuran (10 ml) was added 1,1′-azodicarbonyldipiperidine (399 mg) at room temperature and the mixture was stirred for 2.5 days. The reaction solution was concentrated. The residue was subjected to silica gel column chromatography, and a white solid was obtained from a fraction eluted with ethyl acetate-hexane (1:7, volume ratio). A mixture of the obtained solid, 1N aqueous sodium hydroxide solution (25 ml), tetrahydrofuran (25 ml) and ethanol (25 ml) was stirred overnight at room temperature. 1N Hydrochloric acid (25 ml) was added and the mixture was extracted with ethyl acetate. The ethyl acetate layer was washed with saturated aqueous sodium chloride solution, dried (MgSO₄) and concentrated. The obtained white solid was recrystallized from ethyl acetate-hexane to give 3-[4-(3-{3-ethoxy-1-[5-(trifluoromethyl)-2-pyridinyl]-1H-pyrazol-4-yl}propoxy)-2-propoxyphenyl]propanoic acid (259 mg, yield 63%) as colorless crystals. melting point: 126-127° C.

### Example 118

To a mixture of ethyl 3-[4-(3-{3-ethoxy-1-[5-(trifluoromethyl)-2-pyridinyl]-1H-pyrazol-4-yl}propoxy)-2-hydroxyphenyl]propanoate (470 mg), butanol (170 μL), tributylphosphine (461 μL) and tetrahydrofuran (20 ml) was added 1,1′-azodicarbonyldipiperidine (467 mg) at room temperature and the mixture was stirred overnight. The reaction solution was concentrated. The residue was subjected to silica gel column chromatography, and a colorless oil was obtained from a fraction eluted with ethyl acetate-hexane (1:6, volume ratio). A mixture of the obtained oily substance, 1N aqueous sodium hydroxide solution (25 ml), tetrahydrofuran (25 ml) and ethanol (25 ml) was stirred overnight at room temperature. 1N Hydrochloric acid (25 ml) was added and the mixture was extracted with ethyl acetate. The ethyl acetate layer was washed with saturated aqueous sodium chloride solution, dried (MgSO₄) and concentrated. The obtained white solid was recrystallized from ethyl acetate-hexane to give 3-[2-butoxy-4-{3-(3-ethoxy-1-[5-(trifluoromethyl)-2-pyridinyl]-1H-pyrazol-4-yl}propoxy)phenyl]propanoic acid (235 mg, yield 47%) as colorless crystals. melting point: 123-124° C.

### Example 119

To a mixture of 3-{3-ethoxy-1-[5-(trifluoromethyl)-2-pyridinyl]-1H-pyrazol-4-yl}-1-propanol (463 mg), methyl (3-hydroxy-1-methyl-1H-pyrazol-5-yl)acetate (250 mg), tributylphosphine (728 μL) and tetrahydrofuran (30 ml) was added 1,1′-azodicarbonyldipiperidine (764 mg) at room temperature and the mixture was stirred overnight. The reaction solution was concentrated. The residue was subjected to silica gel column chromatography, and a yellow oily substance was obtained from a fraction eluted with ethyl acetate-hexane (1:2, volume ratio). A mixture of the obtained oily substance, 1N aqueous sodium hydroxide solution (25 ml), tetrahydrofuran (25 ml) and ethanol (25 ml) was stirred overnight at room temperature. 1N Hydrochloric acid (25 ml) was added and the mixture was extracted with ethyl acetate. The ethyl acetate layer was washed with saturated aqueous sodium chloride solution, dried (MgSO₄) and concentrated. The obtained pale-yellow crystals were recrystallized from ethyl acetate-hexane to give [3-(3-{3-ethoxy-1-[5-(trifluoromethyl)-2-pyridinyl]-1H-pyrazol-4-yl}propoxy)-1-methyl-1H-pyrazol-5-yl]acetic acid (255 mg, yield 39%) as colorless crystals. melting point: 151-152° C.

### Example 120

To a mixture of {3-propyl-1-[5-(trifluoromethyl)-2-pyridyl]-1H-pyrazol-4-yl}methanol (250 mg), methyl 3-(4-hydroxy-2-methoxyphenyl)propionate (180 mg), triphenylphosphine (280 mg) and tetrahydrofuran (10 ml) was dropwise added a 40% solution (460 mg) of diethyl azodicarboxylate in toluene at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give 3-[2-methoxy-4-({3-propyl-1-[5-(trifluoromethyl)-2-pyridyl]-1H-pyrazol-4-yl}methoxy)phenyl]propionic acid (210 mg, yield 53%). The crystals were recrystallized from ethyl acetate-hexane. melting point: 153-154° C.

### Example 121

To a mixture of {3-propyl-1-[5-(trifluoromethyl)-2-pyridyl]-1H-pyrazol-4-yl}methanol (410 mg), ethyl 3-(4-hydroxy-2-methylphenyl)propionate (300 mg), triphenylphosphine (450 mg) and tetrahydrofuran (10 ml) was dropwise added a 40% solution (750 mg) of diethyl azodicarboxylate in toluene at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give 3-[2-methyl-4-({3-propyl-1-[5-(trifluoromethyl)-2-pyridyl]-1H-pyrazol-4-yl}methoxy)phenyl]propionic acid (460 mg, yield 72%). The crystals were recrystallized from ethyl acetate-hexane. melting point: 129-130° C.

### Example 122

To a mixture of {3-methyl-1-[5-(trifluoromethyl)-2-pyridyl]-1H-pyrazol-4-yl}methanol (220 mg), methyl 3-(4-hydroxy-2-methoxyphenyl)propionate (180 mg), triphenylphosphine (260 mg) and tetrahydrofuran (10 ml) was dropwise added a 40% solution (450 mg) of diethyl azodicarboxylate in toluene at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give 3-[2-methoxy-4-({3-methyl-1-[5-(trifluoromethyl)-2-pyridyl]-1H-pyrazol-4-yl}methoxy)phenyl]propionic acid (220 mg, yield 59%). The crystals were recrystallized from ethyl acetate-hexane. melting point: 158-159° C.

### Example 123

To a mixture of {3-methyl-1-[5-(trifluoromethyl)-2-pyridyl]-1H-pyrazol-4-yl}methanol (380 mg), ethyl 3-(4-hydroxy-2-methylphenyl)propionate (300 mg), triphenylphosphine (450 mg) and tetrahydrofuran (10 ml) was dropwise added a 40% solution (450 mg) of diethyl azodicarboxylate in toluene at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give 3-[2-methyl-4-({3-methyl-1-[5-(trifluoromethyl)-2-pyridyl]-1H-pyrazol-4-yl}methoxy)phenyl]propionic acid (380 mg, yield 63%). The crystals were recrystallized from ethyl acetate-hexane. melting point: 144-145° C.

### Example 124

To a mixture of 3-{3-phenyl-1-[5-(trifluoromethyl)-2-pyridyl]-1H-pyrazol-4-yl}-1-propanol (400 mg), methyl 3-hydroxyphenylacetate (200 mg), tributylphosphine (480 mg) and tetrahydrofuran (30 ml) was added 1,1′-azodicarbonyldipiperidine (600 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give [3-(3-{3-phenyl-1-[5-(trifluoromethyl)-2-pyridyl]-1H-pyrazol-4-yl}propoxy)phenyl]acetic acid (520 mg, yield 94%). The crystals were recrystallized from ethyl acetate-hexane. melting point: 132-133° C.

### Example 125

To a mixture of 3-{3-phenyl-1-[5-(trifluoromethyl)-2-pyridyl]-1H-pyrazol-4-yl}-1-propanol (420 mg), ethyl 3-(3-hydroxy-1-phenyl-1H-pyrazol-5-yl)propionate (320 mg), tributylphosphine. (500 mg) and tetrahydrofuran (30 ml) was added 1,1′-azodicarbonyldipiperidine (630 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give 3-[1-phenyl-3-(3-{3-phenyl-1-[5-(trifluoromethyl)-2-pyridyl]-1H-pyrazol-4-yl}propoxy)-1H-pyrazol-5-yl]propionic acid (640 mg, yield 94%). The crystals were recrystallized from ethyl acetate-hexane. melting point: 175-176° C.

### Example 126

To a mixture of 3-{3-methyl-1-[5-(trifluoromethyl)-2-pyridyl]-1H-pyrazol-4-yl}-1-propanol (420 mg), methyl 3-(2-ethoxy-4-hydroxyphenyl)propionate (330 mg), tributylphosphine (600 mg) and tetrahydrofuran (30 ml) was added 1,1′-azodicarbonyldipiperidine (750 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give 3-[2-ethoxy-4-(3-{3-methyl-1-[5-(trifluoromethyl)-2-pyridyl]-1H-pyrazol-4-yl}propoxy)phenyl]propionic acid (510 mg, yield 73%). The crystals were recrystallized from ethyl acetate-hexane. melting point: 129-130° C.

### Example 127

To a mixture of 3-{3-methyl-1-[5-(trifluoromethyl)-2-pyridyl]-1H-pyrazol-4-yl}-1-propanol (400 mg), methyl 3-hydroxyphenylacetate (240 mg), tributylphosphine (600 mg) and tetrahydrofuran (30 ml) was added 1,1′-azodicarbonyldipiperidine (750 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give [3-(3-{3-methyl-1-[5-(trifluoromethyl)-2-pyridyl]-1H-pyrazol-4-yl}propoxy)phenyl]acetic acid (550 mg, yield 93%). The crystals were recrystallized from ethyl acetate-hexane. melting point: 96-97° C.

### Example 128

To a mixture of 3-{3-methyl-1-[5-(trifluoromethyl)-2-pyridyl]-1H-pyrazol-4-yl}-1-propanol (420 mg), ethyl 3-(3-hydroxy-1-phenyl-1H-pyrazol-5-yl)propionate (390 mg), tributylphosphine (600 mg) and tetrahydrofuran (30 ml) was added 1,1′-azodicarbonyldipiperidine (750 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give 3-[3-(3-{3-methyl-1-[5-(trifluoromethyl)-2-pyridyl]-1H-pyrazol-4-yl}propoxy)-1-phenyl-1H-pyrazol-5-yl]propionic acid (700 mg, yield 95%). The crystals were recrystallized from ethyl acetate-hexane. melting point: 125-126° C.

### Example 129

To a mixture of 3-{3-methyl-1-[5-(trifluoromethyl)-2-pyridyl]-1H-pyrazol-4-yl}-1-propanol (420 mg), ethyl 3-(4-hydroxy-3-methoxyphenyl)propionate (330 mg), tributylphosphine (600 mg) and tetrahydrofuran (30 ml) was added 1,1′-azodicarbonyldipiperidine (750 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give 3-[3-methoxy-4-(3-{3-methyl-1-[5-(trifluoromethyl)-2-pyridyl]-1H-pyrazol-4-yl}propoxy)phenyl]propionic acid (510 mg, yield 75%). The crystals were recrystallized from ethyl acetate-hexane. melting point: 136-137° C.

### Example 130

To a mixture of 3-{3-methyl-1-[5-(trifluoromethyl)-2-pyridyl]-1H-pyrazol-4-yl}-1-propanol (420 mg), ethyl 2-(3-hydroxyphenoxy)-2-methylpropionate (340 mg), tributylphosphine (600 mg) and tetrahydrofuran (30 ml) was added 1,1′-azodicarbonyldipiperidine (750 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give 2-methyl-2-[3-(3-{3-methyl-1-[5-(trifluoromethyl)-2-pyridyl]-1H-pyrazol-4-yl}propoxy)phenoxy]propionic acid (520 mg, yield 76%). The crystals were recrystallized from ethyl acetate-hexane. melting point: 107-108° C.

### Example 131

To a mixture of 3-{3-ethoxy-1-[5-(trifluoromethyl)-2-pyridyl]-1H-pyrazol-4-yl}-1-propanol (460 mg), ethyl 3-hydroxy-4-methoxyphenylacetate (310 mg), tributylphosphine (600 mg) and tetrahydrofuran (30 ml) was added 1,1′-azodicarbonyldipiperidine (750 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and ethanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give [3-(3-{3-ethoxy-1-[5-(trifluoromethyl)-2-pyridyl]-1H-pyrazol-4-yl}propoxy)-4-methoxyphenyl]acetic acid (560 mg, yield 80%). The crystals were recrystallized from ethyl acetate-hexane. melting point: 142-143° C.

### Example 132

To a mixture of 3-{3-ethoxy-1-[5-(trifluoromethyl)-2-pyridyl]-1H-pyrazol-4-yl}-1-propanol (400 mg), methyl 2-hydroxy-5-methoxyphenylacetate (250 mg), tributylphosphine (520 mg) and tetrahydrofuran (30 ml) was added 1,1′-azodicarbonyldipiperidine (650 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give [2-(3-{3-ethoxy-1-[5-(trifluoromethyl)-2-pyridyl]-1H-pyrazol-4-yl}propoxy)-5-methoxyphenyl]acetic acid (560 mg, yield 92%). The crystals were recrystallized from ethyl acetate-hexane. melting point: 138-139° C.

### Example 133

To a mixture of 3-{3-phenyl-1-[5-(trifluoromethyl)-2-pyridyl]-1H-pyrazol-4-yl}-1-propanol (400 mg), ethyl 2-(3-hydroxyphenoxy)-2-methylpropionate (260 mg), tributylphosphine (480 mg) and tetrahydrofuran (30 ml) was added 1,1′-azodicarbonyldipiperidine (600 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give 2-methyl-2-[3-(3-{3-phenyl-1-[5-(trifluoromethyl)-2-pyridyl]-1H-pyrazol-4-yl}propoxy)phenoxy]propionic acid (540 mg, yield 80%). The crystals were recrystallized from ethyl acetate-hexane. melting point: 141-142° C.

### Example 134

To a mixture of 3-{3-phenyl-1-[5-(trifluoromethyl)-2-pyridyl]-1H-pyrazol-4-yl}-1-propanol (500 mg), methyl 2-hydroxyphenylacetate (240 mg), tributylphosphine (600 mg) and tetrahydrofuran (30 ml) was added 1,1′-azodicarbonyldipiperidine (750 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give [2-(3-{3-phenyl-1-[5-(trifluoromethyl)-2-pyridyl]-1H-pyrazol-4-yl}propoxy)phenyl]acetic acid (510 mg, yield 74%). The crystals were recrystallized from ethyl acetate-hexane. melting point: 133-134° C.

### Example 135

To a mixture of 3-{3-ethoxy-1-[5-(trifluoromethyl)-2-pyridyl]-1H-pyrazol-4-yl}-1-propanol (400 mg), methyl (3-hydroxy-1-methyl-1H-pyrazol-4-yl)acetate (220 mg), tributylphosphine (520 mg) and tetrahydrofuran (30 ml) was added 1,1′-azodicarbonyldipiperidine (650 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give [3-(3-{3-ethoxy-1-[5-(trifluoromethyl)-2-pyridyl]-1H-pyrazol-4-yl}propoxy)-1-methyl-1H-pyrazol-4-yl]acetic acid (440 mg, yield 76%). The crystals were recrystallized from ethyl acetate-hexane. melting point: 115-116° C.

### Example 136

To a mixture of 3-{3-ethoxy-1-[5-(trifluoromethyl)-2-pyridyl]-1H-pyrazol-4-yl}-1-propanol (400 mg), methyl (3-hydroxy-1-phenyl-1H-pyrazol-4-yl)acetate (300 mg), tributylphosphine (520 mg) and tetrahydrofuran (30 ml) was added 1,1′-azodicarbonyldipiperidine (650 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give [3-(3-{3-ethoxy-1-[5-(trifluoromethyl)-2-pyridyl]-1H-pyrazol-4-yl}propoxy)-1-phenyl-1H-pyrazol-4-yl]acetic acid (580 mg, yield 89%). The crystals were recrystallized from ethyl acetate-hexane. melting point: 117-118° C.

### Example 137

To a mixture of methyl {2-[4-(3-ethoxy-1H-pyrazol-4-yl)butoxy]phenyl}acetate (550 mg), 2-chloro-5-(trifluoromethyl)pyridine (300 mg) and N,N-dimethylformamide (5 ml) was added sodium hydride (60%, in oil, 70 mg) at 0° C., and the mixture was stirred overnight at room temperature. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were ollected by filtration to give [2-(4-{3-ethoxy-1-[5-(trifluoromethyl)-2-pyridyl]-1H-pyrazol-4-yl}butoxy)phenyl]acetic acid (100 mg, yield 13%). The crystals ere recrystallized from ethyl acetate-hexane. melting point: 109-110° C.

### Example 138

A mixture of methyl {2-[4-(3-ethoxy-1H-pyrazol-4-yl)butoxy]phenyl}acetate (1.52 g), 4-(trifluoromethyl)phenylboric acid (1.74 g), copper(II) acetate (1.25 g), pyridine (0.67 ml) and N,N-dimethylformamide (20 ml) was stirred at room temperature for 3 days. The reaction mixture was poured into dilute hydrochloric acid, and extracted with ethyl acetate. The ethyl acetate layer was washed with saturated aqueous sodium chloride solution, dried (MgSO₄) and concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (10 ml), tetrahydrofuran (10 ml) and methanol (10 ml) was stirred at room temperature for 1 hour. 1N Hydrochloric acid (10 ml) was added, and the mixture was extracted with ethyl acetate. The ethyl acetate layer was washed with saturated aqueous sodium chloride solution, dried (MgSO₄) and concentrated. A mixture of the obtained oily substance, 1N aqueous sodium hydroxide solution (4 ml) and methanol (5 ml) was stirred at room temperature for 30 minutes. After concentration, water (15 ml) was added. A solution of calcium chloride (350 mg) in water (5 ml) was slowly added while stirring the mixture at room temperature. The obtained colorless amorphous was removed by filtration to give calcium [2-(4-{3-ethoxy-1-[4-(trifluoromethyl)phenyl]-1H-pyrazol-4-yl}butoxy)phenyl]acetate (830 mg, yield 38%).

### Example 139

To a mixture of 3-{3-ethoxy-1-[5-(trifluoromethyl)-2-pyridyl]-1H-pyrazol-4-yl}-1-propanol (400 mg), methyl 5-chloro-2-hydroxyphenylacetate (260 mg), tributylphosphine (520 mg) and tetrahydrofuran (30 ml) was added 1,1′-azodicarbonyldipiperidine (650 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give [5-chloro-2-(3-{3-ethoxy-1-[5-(trifluoromethyl)-2-pyridyl]-1H-pyrazol-4-yl}propoxy)phenyl]acetic acid (580 mg, yield 94%). The crystals were recrystallized from ethyl acetate-hexane. melting point: 130-131° C.

### Example 140

To a mixture of 3-{3-ethoxy-1-[5-(trifluoromethyl)-2-pyridyl]-1H-pyrazol-4-yl}-1-propanol (400 mg), ethyl 3-(2-hydroxy-5-methoxyphenyl)propionate (290 mg), tributylphosphine (520 mg) and tetrahydrofuran (30 ml) was added 1,1′-azodicarbonyldipiperidine (650 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and ethanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give 3-[2-(3-{3-ethoxy-1-[5-(trifluoromethyl)-2-pyridyl]-1H-pyrazol-4-yl}propoxy)-5-ethoxyphenyl]propionic acid (470 mg, yield 75%). The crystals were recrystallized from ethyl acetate-hexane. melting point: 104-105° C.

### Example 141

To a mixture of 3-{3-methyl-1-[5-(trifluoromethyl)-2-pyridyl]-1H-pyrazol-4-yl}-1-propanol (400 mg), methyl 2-hydroxyphenylacetate (240 mg), tributylphosphine (580 mg) and tetrahydrofuran (30 ml) was added 1,1′-azodicarbonyldipiperidine (720 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give [2-(3-{3-methyl-1-[5-(trifluoromethyl)-2-pyridyl]-1H-pyrazol-4-yl}propoxy)phenyl]acetic acid (410 mg, yield 70%). The crystals were recrystallized from ethyl acetate-hexane. melting point: 128-129° C.

### Example 142

To a mixture of 3-{3-ethoxy-1-[5-(trifluoromethyl)-2-pyridyl]-1H-pyrazol-4-yl}-1-propanol (300 mg), methyl 2-hydroxy-4-methoxyphenylacetate (190 mg), tributylphosphine (400 mg) and tetrahydrofuran (30 ml) was added 1,1′-azodicarbonyldipiperidine (510 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give [2-(3-{3-ethoxy-1-[5-(trifluoromethyl)-2-pyridyl]-1H-pyrazol-4-yl}propoxy)-4-methoxyphenyl]acetic acid (310 mg, yield 68%). The crystals were recrystallized from ethyl acetate-hexane. melting point: 147-148° C.

### Example 143

To a mixture of 3-{3-ethoxy-1-[5-(trifluoromethyl)-2-pyridyl]-1H-pyrazol-4-yl}-1-propanol (300 mg), ethyl 3-(2-hydroxy-4-methoxyphenyl)propionate (220 mg), tributylphosphine (400 mg) and tetrahydrofuran (30 ml) was added 1,1′-azodicarbonyldipiperidine (510 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and ethanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give 3-[2-(3-{3-ethoxy-1-[5-(trifluoromethyl)-2-pyridyl]-1H-pyrazol-4-yl}propoxy)-4-methoxyphenyl]propionic acid (340 mg, yield 73%). The crystals were recrystallized from ethyl acetate-hexane. melting point: 115-116° C.

### Example 144

To a mixture of 3-{3-ethoxy-1-[5-(trifluoromethyl)-2-pyridyl]-1H-pyrazol-4-yl}-1-propanol (300 mg), methyl 3-(2-hydroxyphenyl)propionate (180 mg), tributylphosphine (400 mg) and tetrahydrofuran (30 ml) was added 1,1′-azodicarbonyldipiperidine (510 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give 3-[2-(3-{3-ethoxy-1-[5-(trifluoromethyl)-2-pyridyl]-1H-pyrazol-4-yl}propoxy)phenyl]propionic acid (360 mg, yield 82%). The crystals were recrystallized from ethyl acetate-hexane. melting point: 93-94° C.

### Example 145

To a mixture of 3-{3-ethoxy-1-[5-(trifluoromethyl)-2-pyridyl]-1H-pyrazol-4-yl}-1-propanol (300 mg), ethyl 3-(2-hydroxy-3-methoxyphenyl)propionate (220 mg), tributylphosphine (400 mg) and tetrahydrofuran (30 ml) was added 1,1′-azodicarbonyldipiperidine (510 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and ethanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give 3-[2-(3-{3-ethoxy-1-[5-(trifluoromethyl)-2-pyridyl]-1H-pyrazol-4-yl}propoxy)-3-methoxyphenyl]propionic acid (360 mg, yield 77%). The crystals were recrystallized from ethyl acetate-hexane. melting point: 87-88° C.

### Example 146

To a mixture of 3-{3-ethoxy-1-[5-(trifluoromethyl)-2-pyridyl]-1H-pyrazol-4-yl}-1-propanol (300 mg), methyl (2-hydroxy-3-methoxyphenyl)acetate (200 mg), tributylphosphine (400 mg) and tetrahydrofuran (30 ml) was added 1,1′-azodicarbonyldipiperidine (510 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give [2-(3-{3-ethoxy-1-[5-(trifluoromethyl)-2-pyridyl]-1H-pyrazol-4-yl}propoxy)-3-methoxyphenyl]acetic acid (190 mg, yield 42%). The crystals were recrystallized from ethyl acetate-hexane. melting point: 122-123° C.

### Example 147

To a mixture of {3-propyl-1-[5-(trifluoromethyl)-2-pyridyl]-1H-pyrazol-4-yl}methanol (300 mg), methyl 3-hydroxyphenylacetate (190 mg), tributylphosphine (430 mg) and tetrahydrofuran (30 ml) was added 1,1′-azodicarbonyldipiperidine (550 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give [3-({3-propyl-1-[5-(trifluoromethyl)-2-pyridyl]-1H-pyrazol-4-yl}methoxy)phenyl]acetic acid (340 mg, yield 77%). The crystals were recrystallized from ethyl acetate-hexane. melting point: 77-78° C.

### Example 148

To a mixture of 3-{3-ethoxy-1-[5-(trifluoromethyl)-2-pyridyl]-1H-pyrazol-4-yl}-1-propanol (300 mg), ethyl 3-(2-hydroxy-6-methoxyphenyl)propionate (220 mg), tributylphosphine (400 mg) and tetrahydrofuran (30 ml) was added 1,1′-azodicarbonyldipiperidine (510 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and ethanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give 3-[2-(3-{3-ethoxy-1-[5-(trifluoromethyl)-2-pyridyl]-1H-pyrazol-4-yl}propoxy)-6-methoxyphenyl]propionic acid (170 mg, yield 36%). The crystals were recrystallized from ethyl acetate-hexane. melting point: 110-111° C.

### Example 149

To a mixture of {3-methyl-1-[5-(trifluoromethyl)-2-pyridyl]-1H-pyrazol-4-yl}methanol (300 mg), methyl 3-hydroxyphenylacetate (250 mg), tributylphosphine (480 mg) and tetrahydrofuran (30 ml) was added 1,1′-azodicarbonyldipiperidine (600 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give [3-({3-methyl-1-[5-(trifluoromethyl)-2-pyridyl]-1H-pyrazol-4-yl}methoxy)phenyl]acetic acid (330 mg, yield 72%). The crystals were recrystallized from ethyl acetate-hexane. melting point: 142-143° C.

### Example 150

To a mixture of 3-{3-ethoxy-1-[5-(trifluoromethyl)-2-pyridyl]-1H-pyrazol-4-yl}-1-propanol (400 mg), ethyl 3-(4-hydroxy-3-methoxyphenyl)propanoate (310 mg), tributylphosphine (530 mg) and tetrahydrofuran (30 ml) was added 1,1′-azodicarbonyldipiperidine (650 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (2 ml), tetrahydrofuran (4 ml) and methanol (4 ml) was stirred at room temperature for 5 hours. 1N Hydrochloric acid (2 ml) was added and the mixture was extracted with ethyl acetate. The ethyl acetate layer was washed with saturated aqueous sodium chloride solution, dried (MgSO₄) and concentrated. The obtained colorless crystals were collected by filtration to give 3-[4-(3-{3-ethoxy-1-[5-(trifluoromethyl)-2-pyridyl]-1H-pyrazol-4-yl}propoxy)-3-methoxyphenyl]propanoic acid (440 mg, yield 69%). The crystals were recrystallized from isopropyl ether-hexane. melting point: 131-132° C.

### Example 151

A mixture of methyl 3-[2-ethoxy-4-(3-{3-hydroxy-1-[5-(trifluoromethyl)-2-pyridyl]-1H-pyrazol-4-yl}propoxy)phenyl]propanoate (500 mg), 1N aqueous sodium hydroxide solution (3 ml), tetrahydrofuran (5 ml) and methanol (5 ml) was stirred at room temperature for 5 hours and concentrated. 1N Hydrochloric acid (3 ml) was added and the obtained colorless crystals were collected by filtration, washed with water and acetonitrile and dried to give 3-[2-ethoxy-4-(3-{3-hydroxy-1-[5-(trifluoromethyl)-2-pyridyl]-1H-pyrazol-4-yl}propoxy)phenyl]propanoic acid (470 mg, yield 97%). melting point: 192-194° C.

### Example 152

To a mixture of methyl 3-[2-ethoxy-4-(3-{3-hydroxy-1-[5-(trifluoromethyl)-2-pyridyl]-1H-pyrazol-4-yl}propoxy)phenyl]propanoate (600 mg), methyl iodide (0.11 ml) and N,N-dimethylformamide (6 ml) was added sodium hydride (60%, in oil, 58 mg) at 0° C. and the mixture was stirred at room temperature for 2 hours. The reaction mixture was poured into water, and extracted with ethyl acetate. The ethyl acetate layer was washed with saturated aqueous sodium chloride solution, dried (MgSO₄) and concentrated. The residue was subjected to silica gel column chromatography, and colorless crystals were obtained from a fraction eluted with ethyl acetate-hexane (1:4, volume ratio). A mixture of the obtained crystals, 1N aqueous sodium hydroxide solution (1.5 ml), tetrahydrofuran (4 ml) and methanol (4 ml) was stirred at room temperature for 5 hours. 1N Hydrochloric acid (1.5 ml) was added, and the mixture was extracted with ethyl acetate. The ethyl acetate layer was washed with saturated aqueous sodium chloride solution, dried (MgSO₄) and concentrated. The obtained colorless crystals were collected by filtration to give 3-[2-ethoxy-4-(3-{3-methoxy-1-[5-(trifluoromethyl)-2-pyridyl]-1H-pyrazol-4-yl}propoxy)phenyl]propanoic acid (350 mg, yield 58%). The crystals were recrystallized from isopropyl ether. melting point: 145-146° C.

### Example 153

To a mixture of methyl 3-[2-ethoxy-4-(3-{3-hydroxy-1-[5-(trifluoromethyl)-2-pyridyl]-1H-pyrazol-4-yl}propoxy)phenyl]propanoate (600 mg), 1-iodopropane (0.14 ml) and N,N-dimethylformamide (6 ml) was added sodium hydride (60%, in oil, 58 mg) at 0° C. and the mixture was stirred overnight at room temperature. The reaction mixture was poured into water, and extracted with ethyl acetate. The ethyl acetate layer was washed with saturated aqueous sodium chloride solution, dried (MgSO₄) and concentrated. The residue was subjected to silica gel column chromatography, and colorless crystals were obtained from a fraction eluted with ethyl acetate-hexane (1:5, volume ratio). A mixture of the obtained crystals, 1N aqueous sodium hydroxide solution (1.5 ml), tetrahydrofuran (4 ml) and methanol (4 ml) was stirred at room temperature for 5 hours. 1N Hydrochloric acid (1.5 ml) was added, and the mixture was extracted with ethyl acetate. The ethyl acetate layer was washed with saturated aqueous sodium chloride solution, dried (MgSO₄) and concentrated. The obtained colorless crystals were collected by filtration to give 3-[2-ethoxy-4-(3-{3-propoxy-1-[5-(trifluoromethyl)-2-pyridyl]-1H-pyrazol-4-yl}propoxy)phenyl]propanoic acid (380 mg, yield 60%). The crystals were recrystallized from isopropyl ether. melting point: 112-113° C.

### Example 154

To a mixture of methyl 3-[2-ethoxy-4-(3-{3-hydroxy-1-[5-(trifluoromethyl)-2-pyridyl]-1H-pyrazol-4-yl}propoxy)phenyl]propanoate (600 mg), 2-propanol (0.15 ml), triphenylphosphine (480 mg) and tetrahydrofuran (10 ml) was added diisopropyl azodicarboxylate (370 mg) at room temperature and the mixture was stirred for 3 hours. The reaction solution was concentrated. The residue was subjected to silica gel column chromatography, and colorless crystals were obtained from a fraction eluted with ethyl acetate-hexane (1:6, volume ratio). A mixture of the obtained crystals, 1N aqueous sodium hydroxide solution (2 ml), tetrahydrofuran (4 ml) and methanol (4 ml) was stirred at room temperature for 5 hours. 1N Hydrochloric acid (2 ml) was added and the mixture was extracted with ethyl acetate. The ethyl acetate layer was washed with saturated aqueous sodium chloride solution, dried (MgSO₄) and concentrated. The obtained colorless crystals were collected by filtration to give 3-[2-ethoxy-4-(3-{3-isopropoxy-1-[5-(trifluoromethyl)-2-pyridyl]-1H-pyrazol-4-yl}propoxy)phenyl]propanoic acid (520 mg, yield 82%). The crystals were recrystallized from isopropyl ether. melting point: 128-129° C.

### Example 155

To a mixture of methyl 3-[2-ethoxy-4-(3-{3-hydroxy-1-[5-(trifluoromethyl)-2-pyridyl]-1H-pyrazol-4-yl}propoxy)phenyl]propanoate (600 mg), 1-iodobutane (0.17 ml) and N,N-dimethylformamide (6 ml) was added sodium hydride (60%, in oil, 58 mg) at 0° C. and the mixture was stirred overnight at room temperature. The reaction mixture was poured into water, and extracted with ethyl acetate. The ethyl acetate layer was washed with saturated aqueous sodium chloride solution, dried (MgSO₄) and concentrated. The residue was subjected to silica gel column chromatography, and colorless crystals were obtained from a fraction eluted with ethyl acetate-hexane (1:5, volume ratio). A mixture of the obtained crystals, 1N aqueous sodium hydroxide solution (1.5 ml), tetrahydrofuran (4 ml) and methanol (4 ml) was stirred at room temperature for 5 hours. 1N Hydrochloric acid (1.5 ml) was added, and the mixture was extracted with ethyl acetate. The ethyl acetate layer was washed with saturated aqueous sodium chloride solution, dried (MgSO₄) and concentrated. The obtained colorless crystals were collected by filtration to give 3-[4-(3-{3-butoxy-1-[5-(trifluoromethyl)-2-pyridyl]-1H-pyrazol-4-yl}propoxy)-2-ethoxyphenyl]propanoic acid (320 mg, yield 49%). The crystals were recrystallized from isopropyl ether. melting point: 102-103° C.

### Example 156

A mixture of methyl 3-[4-(3-{3-benzyloxy-1-[5-(trifluoromethyl)-2-pyridyl]-1H-pyrazol-4-yl}propoxy)-2-ethoxyphenyl]propanoate (600 mg), 1N aqueous sodium hydroxide solution (1.5 ml), tetrahydrofuran (4 ml) and methanol (4 ml) was stirred at room temperature for 5 hours. 1N Hydrochloric acid (1.5 ml) was added, and the mixture was extracted with ethyl acetate. The ethyl acetate layer was washed with saturated aqueous sodium chloride solution, dried (MgSO₄) and concentrated. The obtained colorless crystals were collected by filtration to give 3-[4-(3-{3-benzyloxy-1-[5-(trifluoromethyl)-2-pyridyl]-1H-pyrazol-4-yl}propoxy)-2-ethoxyphenyl]propanoic acid (380 mg, yield 65%). The crystals were recrystallized from isopropyl ether. melting point: 106-107° C.

### Example 157

To a mixture of 3-{(3-propyl-1-[5-(trifluoromethyl)-2-pyridyl]-1H-pyrazol-4-yl}-1-propanol (500 mg), ethyl 2-(4-hydroxyphenyl)-2-methylpropanoate (370 mg), tributylphosphine (650 mg) and tetrahydrofuran (30 ml) was added 1,1′-azodicarbonyldipiperidine (810 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 4N aqueous sodium hydroxide solution (1 ml) and methanol (10 ml) was refluxed for 15 hours. After cooling, 1N Hydrochloric acid (5 ml) was added, and the mixture was extracted with ethyl acetate. The ethyl acetate layer was washed with saturated aqueous sodium chloride solution, dried (MgSO₄) and concentrated. The obtained colorless crystals were collected by filtration to give 2-methyl-2-[4-(3-{3-propyl-1-[5-(trifluoromethyl)-2-pyridyl]-1H-pyrazol-4-yl}propoxy)phenyl]propanoic acid (250 mg, yield 54%). The crystals were recrystallized from hexane. melting point: 84-85° C.

### Example 158

To a mixture of 3-{3-ethoxy-1-[5-(trifluoromethyl)-2-pyridyl]-1H-pyrazol-4-yl}-1-propanol (500 mg), ethyl 2-(4-hydroxyphenyl)-2-methylpropanoate (360 mg), tributylphosphine (650 mg) and tetrahydrofuran (30 ml) was added 1,1′-azodicarbonyldipiperidine (810 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 4N aqueous sodium hydroxide solution (1 ml) and methanol (10 ml) was refluxed for 15 hours. After cooling, 1N hydrochloric acid (5 ml) was added, and the mixture was extracted with ethyl acetate. The ethyl acetate layer was washed with saturated aqueous sodium chloride solution, dried (MgSO₄) and concentrated to give 2-[4-(3-{3-ethoxy-1-[5-(trifluoromethyl)-2-pyridyl]-1H-pyrazol-4-yl}propoxy)phenyl]-2-methylpropanoic acid (410 mg, yield 84%) as a yellow oily substance.

¹H-NMR (CDCl₃)δ: 1.40 (3H, t, J=7.1 Hz), 1.58 (6H, s), 2.08 (2H, quintet, J=7.3 Hz), 2.60 (2H, t, J=7.4 Hz), 3.99 (2H, t, J=6.2 Hz), 4.34 (2H, q, J=7.1 Hz), 6.84-6.89 (2H, m), 7.28-7.33 (2H, m), 7.81 (1H, d, J=8.8 Hz), 7.90 (1H, dd, J=8.7, 2.3 Hz), 8.19 (1H, s), 8.54-8.56 (1H, m).

### Example 159

To a mixture of 3-{3-propyl-1-[5-(trifluoromethyl)-2-pyridyl]-1H-pyrazol-4-yl}-1-propanol (500 mg), methyl 2-(3-hydroxyphenyl)-2-methylpropanoate (370 mg), tributylphosphine (650 mg) and tetrahydrofuran (30 ml) was added 1,1′-azodicarbonyldipiperidine (810 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 4N aqueous sodium hydroxide solution (1 ml) and methanol (10 ml) was refluxed for 15 hours. After cooling, 1N hydrochloric acid (5 ml) was added to the reaction mixture, and the mixture was extracted with ethyl acetate. The ethyl acetate layer was washed with saturated aqueous sodium chloride solution, dried (MgSO₄) and concentrated. The obtained colorless crystals were collected by filtration to give 2-methyl-2-[3-(3-{3-propyl-1-[5-(trifluoromethyl)-2-pyridyl]-1H-pyrazol-4-yl}propoxy)phenyl]propanoic acid (320 mg, yield 42%). The crystals were recrystallized from hexane. melting point: 82-83° C.

### Example 160

To a mixture of 3-{3-ethoxy-1-[5-(trifluoromethyl)-2-pyridyl]-1H-pyrazol-4-yl}-1-propanol (500 mg), methyl 2-(3-hydroxyphenyl)-2-methylpropanoate (370 mg), tributylphosphine (650 mg) and tetrahydrofuran (30 ml) was added 1,1′-azodicarbonyldipiperidine (810 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 4N aqueous sodium hydroxide solution (1 ml) and methanol (10 ml) was refluxed for 15 hours. After cooling, 1N hydrochloric acid (5 ml) was added to the reaction mixture, and the mixture was extracted with ethyl acetate. The ethyl acetate layer was washed with saturated aqueous sodium chloride solution, dried (MgSO₄) and concentrated. The obtained colorless crystals were collected by filtration to give 2-[3-(3-{3-ethoxy-1-[5-(trifluoromethyl)-2-pyridyl]-1H-pyrazol-4-yl}propoxy)phenyl]-2-methylpropanoic acid (420 mg, yield 87%). The crystals were recrystallized from hexane. melting point: 131-132° C.

### Example 161

A mixture of ethyl 3-[4-(3-{3-ethoxy-1-[5-(trifluoromethyl)-2-pyridyl]-1H-pyrazol-4-yl}propoxy)-3-hydroxyphenyl]propanoate (500 mg), 1N aqueous sodium hydroxide solution (3 ml), tetrahydrofuran (4 ml) and methanol (4 ml) was stirred at room temperature for 5 hours. 1N Hydrochloric acid (3 ml) was added, and the mixture was extracted with ethyl acetate. The ethyl acetate layer was washed with saturated aqueous sodium chloride solution, dried (MgSO₄) and concentrated. The obtained colorless crystals were collected by filtration to give 3-[4-(3-{3-ethoxy-1-[5-(trifluoromethyl)-2-pyridyl]-1H-pyrazol-4-yl}propoxy)-3-hydroxyphenyl]propanoic acid (330 mg, yield 75%). The crystals were recrystallized from isopropyl ether. melting point: 124-125° C.

### Example 162

A mixture of ethyl 3-[4-(3-{3-ethoxy-1-[5-(trifluoromethyl)-2-pyridyl]-1H-pyrazol-4-yl}propoxy)-3-hydroxyphenyl]propanoate (500 mg), potassium carbonate (160 mg), iodoethane (0.3 ml) and N,N-dimethylformamide (8 ml) was stirred overnight at room temperature. The reaction mixture was poured into water, and extracted with ethyl acetate. The ethyl acetate layer was washed with saturated aqueous sodium chloride solution, dried (MgSO₄) and concentrated. The residue was subjected to silica gel column chromatography, and colorless crystals were obtained from a fraction eluted with ethyl acetate-hexane (1:4, volume ratio). A mixture of the obtained crystals, 1N aqueous sodium hydroxide solution (1.5 ml), tetrahydrofuran (4 ml) and methanol (4 ml) was stirred at room temperature for 5 hours. 1N Hydrochloric acid (1.5 ml) was added, and the mixture was extracted with ethyl acetate. The ethyl acetate layer was washed with saturated aqueous sodium chloride solution, dried (MgSO₄) and concentrated. The obtained colorless crystals were collected by filtration to give 3-[3-ethoxy-4-(3-{3-ethoxy-1-[5-(trifluoromethyl)-2-pyridyl]-1H-pyrazol-4-yl}propoxy)phenyl]propanoic acid (370 mg, yield 75%). The crystals were recrystallized from isopropyl ether-hexane. melting point: 114-115° C.

### Example 163

A mixture of ethyl 3-[4-(3-{3-ethoxy-1-[5-(trifluoromethyl)-2-pyridyl]-1H-pyrazol-4-yl}propoxy)-3-hydroxyphenyl]propanoate (500 mg), potassium carbonate (160 mg), 1-iodopropane (0.2 ml) and N,N-dimethylformamide (8 ml) was stirred overnight at room temperature. The reaction mixture was poured into water, and extracted with ethyl acetate. The ethyl acetate layer was washed with saturated aqueous sodium chloride solution, dried (MgSO₄) and concentrated. The residue was subjected to silica gel column chromatography, and colorless crystals were obtained from a fraction eluted with ethyl acetate-hexane (1:4, volume ratio). A mixture of the obtained crystals, 1N aqueous sodium hydroxide solution (1.5 ml), tetrahydrofuran (4 ml) and methanol (4 ml) was stirred at room temperature for 5 hours. 1N Hydrochloric acid (1.5 ml) was added, and the mixture was extracted with ethyl acetate. The ethyl acetate layer was washed with saturated aqueous sodium chloride solution, dried (MgSO₄) and concentrated. The obtained colorless crystals were collected by filtration to give 3-[4-(3-{3-ethoxy-1-[5-(trifluoromethyl)-2-pyridyl]-1H-pyrazol-4-yl}propoxy)-3-propoxyphenyl]propanoic acid (440 mg, yield 86%). The crystals were recrystallized from isopropyl ether-hexane. melting point: 106-107° C.

### Example 164

To a mixture of ethyl 3-[4-(3-{3-ethoxy-1-[5-(trifluoromethyl)-2-pyridyl]-1H-pyrazol-4-yl}propoxy)-3-hydroxyphenyl]propanoate (200 mg), 2-propanol (0.11 ml), tributylphosphine (400 mg) and tetrahydrofuran (30 ml) was added 1,1′-azodicarbonyldipiperidine (500 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (1.5 ml), tetrahydrofuran (4 ml) and methanol (4 ml) was stirred at room temperature for 15 hours. 1N Hydrochloric acid (1.5 ml) was added, and the mixture was extracted with ethyl acetate. The ethyl acetate layer was washed with saturated aqueous sodium chloride solution, dried (MgSO₄) and concentrated. The obtained colorless crystals were collected by filtration to give 3-[4-(3-{3-ethoxy-1-[5-(trifluoromethyl)-2-pyridyl]-1H-pyrazol-4-yl}propoxy)-3-isopropoxyphenyl]propanoic acid (320 mg, yield 62%). The crystals were recrystallized from isopropyl ether-hexane. melting point: 93-94° C.

### Example 165

A mixture of ethyl 3-[4-(3-{3-ethoxy-1-[5-(trifluoromethyl)-2-pyridyl]-1H-pyrazol-4-yl}propoxy)-3-hydroxyphenyl]propanoate (500 mg), potassium carbonate (160 mg), 1-iodobutane (0.3 ml) and N,N-dimethylformamide (8 ml) was stirred overnight at room temperature. The reaction mixture was poured into water, and extracted with ethyl acetate. The ethyl acetate layer was washed with saturated aqueous sodium chloride solution, dried (MgSO₄) and concentrated. The residue was subjected to silica gel column chromatography, and colorless crystals were obtained from a fraction eluted with ethyl acetate-hexane (1:4, volume ratio). A mixture of the obtained crystals, 1N aqueous sodium hydroxide solution (1.5 ml), tetrahydrofuran (4 ml) and methanol (4 ml) was stirred at room temperature for 5 hours. 1N Hydrochloric acid (1.5 ml) was added, and the mixture was extracted with ethyl acetate. The ethyl acetate layer was washed with saturated aqueous sodium chloride solution, dried (MgSO₄) and concentrated. The obtained colorless crystals were collected by filtration to give 3-[3-butoxy-4-(3-{3-ethoxy-1-[5-(trifluoromethyl)-2-pyridyl]-1H-pyrazol-4-yl}propoxy)phenyl]propanoic acid (450 mg, yield 86%). The crystals were recrystallized from isopropyl ether-hexane. melting point: 92-93° C.

### Example 166

A mixture of ethyl 3-[3-benzyloxy-4-(3-{3-ethoxy-1-[5-(trifluoromethyl)-2-pyridyl]-1H-pyrazol-4-yl}propoxy)phenyl]propanoate (665 mg), 1N aqueous sodium hydroxide solution (2 ml), tetrahydrofuran (4 ml) and methanol (4 ml) was stirred at room temperature for 5 hours. 1N Hydrochloric acid (2 ml) was added and the mixture was extracted with ethyl acetate. The ethyl acetate layer was washed with saturated aqueous sodium chloride solution, dried (MgSO₄) and concentrated. The obtained colorless crystals were collected by filtration to give 3-[3-benzyloxy-4-(3-{3-ethoxy-1-[5-(trifluoromethyl)-2-pyridyl]-1H-pyrazol-4-yl}propoxy)phenyl]propanoic acid (460 mg, yield 71%). The crystals were recrystallized from isopropyl ether. melting point: 115-116° C.

### Example 167

To a mixture of 3-{3-ethoxy-1-[5-(trifluoromethyl)-2-pyridyl]-1H-pyrazol-4-yl}-1-propanol (500 mg), ethyl 3-(3-hydroxy-5-methoxyphenyl)propanoate (380 mg), tributylphosphine (650 mg) and tetrahydrofuran (30 ml) was added 1,1′-azodicarbonyldipiperidine (810 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (2 ml), tetrahydrofuran (4 ml) and methanol (4 ml) was stirred at room temperature for 5 hours. 1N Hydrochloric acid (2 ml) was added and the mixture was extracted with ethyl acetate. The ethyl acetate layer was washed with saturated aqueous sodium chloride solution, dried (MgSO₄) and concentrated. The obtained colorless crystals were collected by filtration to give 3-[5-(3-{3-ethoxy-1-[5-(trifluoromethyl)-2-pyridyl]-1H-pyrazol-4-yl}propoxy)-3-methoxyphenyl]propanoic acid (430 mg, yield 55%). The crystals were recrystallized from isopropyl ether-hexane. melting point: 99-100° C.

### Example 168

To a mixture of 3-{3-ethoxy-1-[5-(trifluoromethyl)-2-pyridyl]-1H-pyrazol-4-yl}-1-propanol (500 mg), methyl 3-hydroxy-5-methoxyphenylacetate (345 mg), tributylphosphine (650 mg) and tetrahydrofuran (30 ml) was added 1,1′-azodicarbonyldipiperidine (810 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (2 ml), tetrahydrofuran (4 ml) and methanol (4 ml) was stirred at room temperature for 5 hours. 1N Hydrochloric acid (2 ml) was added and the mixture was extracted with ethyl acetate. The ethyl acetate layer was washed with saturated aqueous sodium chloride solution, dried (MgSO₄) and concentrated. The obtained colorless crystals were collected by filtration to give [5-(3-{3-ethoxy-1-[5-(trifluoromethyl)-2-pyridyl]-1H-pyrazol-4-yl}propoxy)-3-methoxyphenyl]acetic acid (370 mg, yield 49%). The crystals were recrystallized from isopropyl ether. melting point: 125-126° C.

### Example 169

To a mixture of 3-{3-propyl-1-[5-(trifluoromethyl)-2-S pyridyl]-1H-pyrazol-4-yl}-1-propanol (500 mg), methyl 3-hydroxy-5-methoxyphenylacetate (345 mg), tributylphosphine (650 mg) and tetrahydrofuran (30 ml) was added 1,1′-azodicarbonyldipiperidine (810 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (2 ml), tetrahydrofuran (4 ml) and methanol (4 ml) was stirred at room temperature for 5 hours. 1N Hydrochloric acid (2 ml) was added and the mixture was extracted with ethyl acetate. The ethyl acetate layer was washed with saturated aqueous sodium chloride solution, dried (MgSO₄) and concentrated. The obtained colorless crystals were collected by filtration to give [3-methoxy-5-(3-{3-propyl-1-[5-(trifluoromethyl)-2-pyridyl]-1H-pyrazol-4-yl}propoxy)phenyl]acetic acid (410 mg, yield 54%). The crystals were recrystallized from isopropyl ether. melting point: 139-140° C.

### Example 170

To a mixture of 3-{3-benzyloxy-1-[5-(trifluoromethyl)-2-pyridyl]-1H-pyrazol-4-yl}-1-propanol (9.04 g), methyl 3-(2-ethoxy-4-hydroxyphenyl)propanoate (6.42 g), triphenylphosphine (7.51 g) and tetrahydrofuran (150 ml) was added diisopropyl azodicarboxylate (5.79 g) at room temperature and the mixture was stirred overnight. The reaction solution was concentrated. The residue was subjected to silica gel column chromatography, and was obtained from a fraction eluted with ethyl acetate-hexane (1:5, volume ratio), methyl 3-[4-(3-{3-benzyloxy-1-[5-(trifluoromethyl)-2-pyridyl]-1H-pyrazol-4-yl}propoxy)-2-ethoxyphenyl]propanoate (13.03 g, yield 94%) as a pale-yellow oily substance.

¹H-NMR (CDCl₃)δ: 1.39 (3H, t, J=7.0 Hz), 2.08 (2H, quintet, J=7.2 Hz), 2.55-2.66 (4H, m), 2.86 (2H, t, J=7.7 Hz), 3.65 (3H, s), 3.93-4.00 (4H, m), 5.35 (2H, s), 6.35 (1H, dd, J=8.3, 2.4 Hz), 6.40 (1H, d, J=2.4 Hz), 7.00 (1H, d, J=8.3 Hz), 7.31-7.49 (5H, m), 7.84 (1H, d, J=8.8 Hz), 7.91-7.95 (1H, m), 8.22 (1H, s), 8.55-8.57 (1H, m).

### Example 171

A mixture of methyl 3-[4-(3-{3-benzyloxy-1-[5-(trifluoromethyl)-2-pyridyl]-1H-pyrazol-4-yl}propoxy)-2-ethoxyphenyl]propanoate (12.67 g), 5% palladium-carbon (1.3 g) and ethanol (150 ml) was stirred overnight at room temperature under a hydrogen atmosphere. Palladium-carbon was removed by filtration and the filtrate was concentrated to give methyl 3-[2-ethoxy-4-(3-{3-hydroxy-1-[5-(trifluoromethyl)-2-pyridyl]-1H-pyrazol-4-yl}propoxy)phenyl]propanoate as colorless crystals. melting point: 147-148° C.

### Example 172

To a mixture of 3-{3-ethoxy-1-[5-(trifluoromethyl)-2-pyridyl]-1H-pyrazol-4-yl}-1-propanol (3.00 g), ethyl 3-(3-benzyloxy-4-hydroxyphenyl)propanoate (2.90 g), tributylphosphine (3.84 g) and tetrahydrofuran (100 ml) was added 1,1′-azodicarbonyldipiperidine (4.80 g) at room temperature and the mixture was stirred overnight. The reaction solution was concentrated. The residue was subjected to silica gel column chromatography, and ethyl 3-[3-benzyloxy-4-(3-{3-ethoxy-1-[5-(trifluoromethyl)-2-pyridyl]-1H-pyrazol-4-yl}propoxy)phenyl]propanoate (4.14 g, yield 73%) was obtained as a colorless oil from a fraction eluted with ethyl acetate-hexane (1:4, volume ratio).

¹H-NMR (CDCl₃)δ: 1.22 (3H, t, J=7.1 Hz), 1.40 (3H, t, J=7.1 Hz), 2.13 (2H, quintet, J=6.9 Hz), 2.57 (2H, t, J=7.8 Hz), 2.64 (2H, t, J=7.4 Hz), 2.86 (2H, t, J=7.8 Hz), 4.07 (2H, t, J=6.3 Hz), 4.11 (2H, q, J=7.1 Hz), 4.35 (2H, q, J=7.1 Hz), 5.11 (2H, s), 6.68 (1H, dd, J=8.2, 1.8 Hz), 6.76 (1H, d, J=2.0 Hz), 6.84 (1H, d, J=8.1 Hz), 7.27-7.47 (5H, m), 7.80 (1H, d, J=8.8 Hz), 7.90 (1H, dd, J=8.7, 2.3 Hz), 8.19 (1H, s), 8.53-8.55 (1H, m).

### Example 173

A mixture of ethyl 3-[3-benzyloxy-4-(3-{3-ethoxy-1-[5-(trifluoromethyl)-2-pyridyl]-1H-pyrazol-4-yl}propoxy)phenyl]propanoate (4.14 g), 5% palladium-carbon (0.4 g), tetrahydrofuran (25 ml) and ethanol (25 ml) was stirred overnight at room temperature under a hydrogen atmosphere. Palladium-carbon was removed by filtration and the filtrate was concentrated. The residue was subjected to silica gel column chromatography, and ethyl 3-[4-(3-{3-ethoxy-1-[5-(trifluoromethyl)-2-pyridyl]-1H-pyrazol-4-yl}propoxy)-3-hydroxyphenyl]propanoate (3.25 g, yield 92%) was obtained as colorless crystals from a fraction eluted with ethyl acetate-chloroform (1:20, volume ratio). melting point: 92-93° C.

### Example 174

To a mixture of 3-{3-ethoxy-1-[5-(trifluoromethyl)-2-pyridinyl]-1H-pyrazol-4-yl}-1-propanol (2.69 g), ethyl 3-[2-(benzyloxy)-4-hydroxyphenyl]propanoate (2.56 g), tributylphosphine (4.24 ml) and tetrahydrofuran (180 ml) was added 1,1′-azodicarbonyldipiperidine (4.29 g) at room temperature and the mixture was stirred for 3 days. The reaction solution was concentrated. The residue was subjected to silica gel column chromatography, and ethyl 3-[2-(benzyloxy)-4-(3-{3-ethoxy-1-[5-(trifluoromethyl)-2-pyridinyl]-1H-pyrazol-4-yl}propoxy)phenyl]propanoate (2.79 g, yield 55%) was obtained as a white solid from a fraction eluted with ethyl acetate-hexane (1:6, volume ratio). The obtained solid was recrystallized from ethyl acetate-hexane, colorless crystal. melting point: 80-81° C.

### Example 175

A mixture of ethyl 3-[2-(benzyloxy)-4-(3-{3-ethoxy-1-[5-(trifluoromethyl)-2-pyridinyl]-1H-pyrazol-4-yl}propoxy)phenyl]propanoate (2.44 g), 5% palladium-carbon (1.00 g), tetrahydrofuran (25 ml) and ethanol (50 ml) was stirred overnight at room temperature under a hydrogen atmosphere. Palladium-carbon was removed by filtration and the filtrate was concentrated to give ethyl 3-[4-(3-{3-ethoxy-1-[5-(trifluoromethyl)-2-pyridinyl]-1H-pyrazol-4-yl}propoxy)-2-hydroxyphenyl]propanoate (1.63 g, yield 79%) as a colorless oil.

¹H-NMR (CDCl₃) δ: 1.23 (3H, t, J=6.9 Hz), 1.41 (3H, t, J=6.9 Hz), 2.01-2.12 (2H, m), 2.54-2.62 (2H, m), 2.64-2.71 (2H, m), 2.77-2.84 (2H, m), 3.92-3.98 (2H, m), 4.14 (2H, q, J=6.9 Hz), 4.34 (2H, q, J=6.9 Hz), 6.40-6.48 (2H, m), 6.94 (1H, d, J=8.1 Hz), 7.50 (1H, s), 7.80 (1H, d, J=8.7 Hz), 7.86-7.92 (1H, m), 8.17 (1H, s), 8.52-8.54 (1H, m).

### Example 176

To a mixture of {3-isopropyl-1-[5-(trifluoromethyl)-2-pyridyl]-1H-pyrazol-4-yl}methanol (400 mg), methyl (3-hydroxyphenyl)acetate (300 mg), tributylphosphine (570 mg) and tetrahydrofuran (30 ml) was added 1,1′-azodicarbonyldipiperidine (720 mg) at room temperature and the mixture was stirred overnight. The reaction solution was concentrated and isopropyl ether (20 ml) was added to the residue. The insoluble material was removed by filtration and the filtrate was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give 3-{3-isopropyl-1-[5-(trifluoromethyl)-2-pyridyl]-1H-pyrazol-4-yl}methoxyphenylacetic acid (380 mg, yield 65%). The crystals were recrystallized from ethyl acetate-hexane. melting point: 114-115° C.

### Example 177

To a mixture of 3-[3-ethoxy-1-{5-(trifluoromethyl)-2-pyridyl]-1H-pyrazol-4-yl}-1-propanol (400 mg), methyl (6-hydroxy-2-methoxyphenyl)acetate (260 mg), tributylphosphine (540 mg) and tetrahydrofuran (30 ml) was added 1,1′-azodicarbonyldipiperidine (670 mg) at room temperature and the mixture was stirred overnight. The reaction solution was concentrated and isopropyl ether (20 ml) was added to the residue. The insoluble material was removed by filtration and the filtrate was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give [6-(3-{3-ethoxy-1-[5-(trifluoromethyl)-2-pyridyl]-1H-pyrazol-4-yl}propoxy)-2-methoxyphenyl]acetic acid (490 mg, yield 80%). The crystals were recrystallized from ethyl acetate-hexane. melting point: 150-151° C.

### Example 178

To a mixture of {3-isopropyl-1-[4-(trifluoromethyl)phenyl]-1H-pyrazol-4-yl}methanol (400 mg), methyl (3-hydroxyphenyl)acetate (350 mg), tributylphosphine (570 mg) and tetrahydrofuran (30 ml) was added 1,1′-azodicarbonyldipiperidine (720 mg) at room temperature and the mixture was stirred overnight. The reaction solution was concentrated and isopropyl ether (20 ml) was added to the residue. The insoluble material was removed by filtration and the filtrate was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give [3-({3-isopropyl-1-[4-(trifluoromethyl)phenyl]-1H-pyrazol-4-yl}methoxy)phenyl]acetic acid (360 mg, yield 61%). The crystals were recrystallized from ethyl acetate-hexane. melting point: 92-93° C.

### Example 179

To a mixture of 3-{3-isopropyl-1-[5-(trifluoromethyl)-2-pyridyl]-1H-pyrazol-4-yl}-1-propanol (380 mg), methyl (2-hydroxyphenyl)acetate (210 mg), tributylphosphine (510 mg) and tetrahydrofuran (30 ml) was added 1,1′-azodicarbonyldipiperidine (650 mg) at room temperature and the mixture was stirred overnight. The reaction solution was concentrated and isopropyl ether (20 ml) was added to the residue. The insoluble material was removed by filtration and the filtrate was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give [2-(3-{3-isopropyl-1-[5-(trifluoromethyl)-2-pyridyl]-1H-pyrazol-4-yl}propoxy)phenyl]acetic acid (440 mg, yield 81%). The crystals were recrystallized from ethyl acetate-hexane. melting point: 132-133° C.

### Example 180

To a mixture of 4-{3-isopropyl-1-[5-(trifluoromethyl)-2-pyridyl]-1H-pyrazol-4-yl}-1-butanol (430 mg), methyl (2-hydroxy-3-methoxyphenyl)acetate (260 mg), tributylphosphine (550 mg) and tetrahydrofuran (30 ml) was added 1,1′-azodicarbonyldipiperidine (690 mg) at room temperature and the mixture was stirred overnight. The reaction solution was concentrated and isopropyl ether (20 ml) was added to the residue. The insoluble material was removed by filtration and the filtrate was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give [2-(4-{3-isopropyl-1-[5-(trifluoromethyl)-2-pyridyl]-1H-pyrazol-4-yl}butoxy)-3-methoxyphenyl]acetic acid (550 mg, yield 85%). The crystals were recrystallized from ethyl acetate-hexane. melting point: 144-145° C.

### Example 181

To a mixture of 3-{3-isopropyl-1-[5-(trifluoromethyl)-2-pyridyl]-1H-pyrazol-4-yl}-1-propanol (400 mg), methyl (2-hydroxy-3-methoxyphenyl)acetate (260 mg), tributylphosphine (550 mg) and tetrahydrofuran (30 ml) was added 1,1′-azodicarbonyldipiperidine (690 mg) at room temperature and the mixture was stirred overnight. The reaction solution was concentrated and isopropyl ether (20 ml) was added to the residue. The insoluble material was removed by filtration and the filtrate was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give [2-(3-{3-isopropyl-1-[5-(trifluoromethyl)-2-pyridyl]-1H-pyrazol-4-yl}propoxy)-3-methoxyphenyl]acetic acid (460 mg, yield 75%). The crystals were recrystallized from ethyl acetate-hexane. melting point: 142-143° C.

### Example 182

To a mixture of 3-{3-ethoxy-1-[4-(trifluoromethyl)phenyl]-1H-pyrazol-4-yl}-1-propanol (400 mg), methyl (2-hydroxyphenyl)acetate (230 mg), tributylphosphine (520 mg) and tetrahydrofuran (30 ml) was added 1,1′-azodicarbonyldipiperidine (650 mg) at room temperature and the mixture was stirred overnight. The reaction solution was concentrated and isopropyl ether (20 ml) was added to the residue. The insoluble material was removed by filtration and the filtrate was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give [2-(3-{3-ethoxy-1-[4-(trifluoromethyl)phenyl]-1H-pyrazol-4-yl}propoxy)phenyl]acetic acid (430 mg, yield 76%). The crystals were recrystallized from ethyl acetate-hexane. melting point: 114-115° C.

### Example 183

To a mixture of 3-{3-isopropyl-1-[5-(trifluoromethyl)-2-pyridyl]-1H-pyrazol-4-yl}-1-propanol (400 mg), methyl (3-hydroxy-4-methoxyphenyl)acetate (260 mg), tributylphosphine (550 mg) and tetrahydrofuran (30 ml) was added 1,1′-azodicarbonyldipiperidine (690 mg) at room temperature and the mixture was stirred overnight. The reaction solution was concentrated and isopropyl ether (20 ml) was added to the residue. The insoluble material was removed by filtration and the filtrate was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give [3-(3-{3-isopropyl-1-[5-(trifluoromethyl)-2-pyridyl]-1H-pyrazol-4-yl}propoxy)-4-methoxyphenyl]acetic acid (590 mg, yield 97%). The crystals were recrystallized from ethyl acetate-hexane. melting point: 122-123° C.

### Example 184

To a mixture of 3-{3-isopropyl-1-[4-(trifluoromethyl)phenyl]-1H-pyrazol-4-yl}-1-propanol (450 mg), methyl (2-hydroxyphenyl)acetate (250 mg), tributylphosphine (590 mg) and tetrahydrofuran (30 ml) was added 1,1′-azodicarbonyldipiperidine (750 mg) at room temperature and the mixture was stirred overnight. The reaction solution was concentrated and isopropyl ether (20 ml) was added to the residue. The insoluble material was removed by filtration and the filtrate was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give [2-(3-{3-isopropyl-1-[4-(trifluoromethyl)phenyl]-1H-pyrazol-4-yl}propoxy]phenylacetic acid (560 mg, yield 87%). The crystals were recrystallized from ethyl acetate-hexane. melting point: 89-90° C.

### Example 185

To a mixture of 3-{3-isopropyl-1-[4-(trifluoromethyl)phenyl]-1H-pyrazol-4-yl}-1-propanol (500 mg), methyl (2-hydroxy-3-methoxyphenyl)acetate (330 mg), tributylphosphine (650 mg) and tetrahydrofuran (30 ml) was added 1,1′-azodicarbonyldipiperidine (810 mg) at room temperature and the mixture was stirred overnight. The reaction solution was concentrated and isopropyl ether (20 ml) was added to the residue. The insoluble material was removed by filtration and the filtrate was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give [2-(3-{3-isopropyl-1-[4-(trifluoromethyl)phenyl]-1H-pyrazol-4-yl}propoxy)-3-methoxyphenyl]acetic acid (730 mg, yield 96%). The crystals were recrystallized from ethyl acetate-hexane. melting point: 114-115° C.

### Example 186

To a mixture of 3-{3-ethoxy-1-[5-(trifluoromethyl)-2-pyridyl]-1H-pyrazol-4-yl}-1-propanol (380 mg), methyl (2-fluoro-3-hydroxyphenyl)acetate (240 mg), tributylphosphine (490 mg) and tetrahydrofuran (30 ml) was added 1,1′-azodicarbonyldipiperidine (620 mg) at room temperature and the mixture was stirred overnight. The reaction solution was concentrated and isopropyl ether (20 ml) was added to the residue. The insoluble material was removed by filtration and the filtrate was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give [3-(3-{3-ethoxy-1-[5-(trifluoromethyl)-2-pyridyl]-1H-pyrazol-4-yl}propoxy)-2-fluorophenyl]acetic acid (430 mg, yield 76%). The crystals were recrystallized from ethyl acetate-hexane. melting point: 120-121° C.

### Example 187

To a mixture of {3-cyclohexyl-1-[5-(trifluoromethyl)-2-pyridyl]-1H-pyrazol-4-yl}methanol (380 mg), methyl (3-hydroxyphenyl)acetate (200 mg), tributylphosphine (490 mg) and tetrahydrofuran (30 ml) was added 1,1′-azodicarbonyldipiperidine (610 mg) at room temperature and the mixture was stirred overnight. The reaction solution was concentrated and isopropyl ether (20 ml) was added to the residue. The insoluble material was removed by filtration and the filtrate was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give [3-({3-cyclohexyl-1-[5-(trifluoromethyl)-2-pyridyl]-1H-pyrazol-4-yl}methoxy)phenyl]acetic acid (480 mg, yield 89%). The crystals were recrystallized from ethyl acetate-hexane. melting point: 129-130° C.

### Example 188

To a mixture of {3-cyclohexyl-1-[5-(trifluoromethyl)-2-pyridyl]-1H-pyrazol-4-yl}methanol (430 mg), methyl (2-hydroxyphenyl)acetate (230 mg), tributylphosphine (530 mg) and tetrahydrofuran (30 ml) was added 1,1′-azodicarbonyldipiperidine (670 mg) at room temperature and the mixture was stirred overnight. The reaction solution was concentrated and isopropyl ether (20 ml) was added to the residue. The insoluble material was removed by filtration and the filtrate was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give [2-({3-cyclohexyl-1-[5-(trifluoromethyl)-2-pyridyl]-1H-pyrazol-4-yl}methoxy)phenyl]acetic acid (310 mg, yield 51%). The crystals were recrystallized from ethyl acetate-hexane. melting point: 131-132° C.

### Example 189

To a mixture of 3-{3-cyclohexyl-1-[5-(trifluoromethyl)-2-pyridyl]-1H-pyrazol-4-yl}-1-propanol (400 mg), ethyl 3-(3-hydroxy-1-phenyl-1H-pyrazol-5-yl)propionate (300 mg), tributylphosphine (470 mg) and tetrahydrofuran (30 ml) was added 1,1′-azodicarbonyldipiperidine (590 mg) at room temperature and the mixture was stirred overnight. The reaction solution was concentrated and isopropyl ether (20 ml) was added to the residue. The insoluble material was removed by filtration and the filtrate was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and ethanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give 3-[3-(3-{3-cyclohexyl-1-[5-(trifluoromethyl)-2-pyridyl]-1H-pyrazol-4-yl}propoxy)-1-phenyl-1H-pyrazol-5-yl]propionic acid (190 mg, yield 30%). The crystals were recrystallized from ethyl acetate-hexane. melting point: 182-183° C.

### Example 190

To a mixture of {3-cyclohexyl-1-[5-(trifluoromethyl)-2-pyridyl]-1H-pyrazol-4-yl}methanol (400 mg), methyl (3-hydroxy-4-methoxyphenyl)acetate (250 mg), tributylphosphine (530 mg) and tetrahydrofuran (30 ml) was added 1,1′-azodicarbonyldipiperidine (660 mg) at room temperature and the mixture was stirred overnight. The reaction solution was concentrated and isopropyl ether (20 ml) was added to the residue. The insoluble material was removed by filtration and the filtrate was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give [3-({3-cyclohexyl-1-[5-(trifluoromethyl)-2-pyridyl]-1H-pyrazol-4-yl}methoxy-4-methoxy)phenyl]acetic acid (240 mg, yield 40%). The crystals were recrystallized from ethyl acetate-hexane. melting point: 145-146° C.

### Example 191

To a mixture of {3-cyclohexyl-1-[5-(trifluoromethyl)-2-pyridyl]-1H-pyrazol-4-yl}methanol (400 mg), methyl 3-(3-hydroxyphenyl)propionate (230 mg), tributylphosphine (520 mg) and tetrahydrofuran (30 ml) To a mixture of was added 1,1′-azodicarbonyldipiperidine (660 mg) at room temperature and the mixture was stirred overnight. The reaction solution was concentrated and isopropyl ether (20 ml) was added to the residue. The insoluble material was removed by filtration and the filtrate was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture as extracted with ethyl acetate. The ethyl acetate layer was washed with saturated aqueous sodium chloride solution, dried (MgSO₄) and concentrated. The obtained colorless crystals were collected by filtration to give 3-(3-{3-cyclohexyl-1-[5-(trifluoromethyl)-2-pyridyl]-1H-pyrazol-4-yl}methoxyphenyl)propionic acid (300 mg, yield 52%). The crystals were recrystallized from ethyl acetate-hexane. melting point: 94-95° C.

### Example 192

To a mixture of {3-cyclohexyl-1-[5-(trifluoromethyl)-2-pyridyl]-1H-pyrazol-4-yl}methanol (400 mg), ethyl 3-(3-hydroxy-1-methyl-1H-pyrazol-5-yl)propionate (250 mg), tributylphosphine (510 mg) and tetrahydrofuran (30 ml) was added 1,1′-azodicarbonyldipiperidine (640 mg) at room temperature and the mixture was stirred overnight. The reaction solution was concentrated and isopropyl ether (20 ml) was added to the residue. The insoluble material was removed by filtration and the filtrate was concentrated. The residue was subjected to silica gel column chromatography, and a colorless oil was obtained from a fraction eluted with ethyl acetate-hexane (1:2, volume ratio). A mixture of the obtained oily substance, 1N aqueous sodium hydroxide solution (5 ml), tetrahydrofuran (5 ml) and ethanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give 3-(3-{3-cyclohexyl-1-[5-(trifluoromethyl)-2-pyridyl]-1H-pyrazol-4-yl}methoxy-1-methyl-1H-pyrazol-5-yl)}propionic acid (380 mg, yield 65%). The crystals were recrystallized from ethyl acetate-hexane. melting point: 162-163° C.

### Example 193

To a mixture of {3-cyclohexyl-1-[5-(trifluoromethyl)-2-pyridyl]-1H-pyrazol-4-yl}methanol (400 mg), ethyl 3-(3-hydroxy-1-methyl-1H-pyrazol-4-yl)propionate (250 mg), tributylphosphine (510 mg) and tetrahydrofuran (30 ml) was added 1,1′-azodicarbonyldipiperidine (640 mg) at room temperature and the mixture was stirred overnight. The reaction solution was concentrated and isopropyl ether (20 ml) was added to the residue. The insoluble material was removed by filtration and the filtrate was concentrated. The residue was subjected to silica gel column chromatography, and a colorless oil was obtained from a fraction eluted with ethyl acetate-hexane (1:2, volume ratio). A mixture of the obtained oily substance, 1N aqueous sodium hydroxide solution (5 ml), tetrahydrofuran (5 ml) and ethanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give 3-(3-{3-cyclohexyl-1-[(5-(trifluoromethyl)-2-pyridyl]-1H-pyrazol-4-yl}methoxy-1-methyl-1H-pyrazol-4-yl)propionic acid (410 mg, yield 70%). The crystals were recrystallized from ethyl acetate-hexane. melting point: 143-144° C.

### Example 194

To a mixture of 3-{3-(1-methylethyl)-1-[5-(trifluoromethyl)-1,3,4-thiadiazol-2-yl]-1H-pyrazol-4-yl}-1-propanol (400 mg), methyl (2-hydroxy-3-methoxyphenyl)acetate (270 mg), tributylphosphine (510 mg) and tetrahydrofuran (30 ml) was added 1,1′-azodicarbonyldipiperidine (630 mg) at room temperature and the mixture was stirred overnight. The reaction solution was concentrated. The residue was subjected to silica gel column chromatography, and a colorless oil was obtained from a fraction eluted with ethyl acetate-hexane (1:2, volume ratio). A mixture of the obtained oily substance, 1N aqueous sodium hydroxide solution (2 ml), tetrahydrofuran (4 ml) and methanol (4 ml) was stirred at room temperature for 5 hours. 1N Hydrochloric acid (2 ml) was added and the mixture was extracted with ethyl acetate. The ethyl acetate layer was washed with saturated aqueous sodium chloride solution, dried (MgSO₄) and concentrated. The obtained colorless crystals were collected by filtration to give [3-methoxy-2-(3-{3-(1-methylethyl)-1-[5-(trifluoromethyl)-1,3,4-thiadiazole-2-yl]-1H-pyrazol-4-yl}propoxy)phenyl]acetic acid (130 mg, yield 22%). The crystals were recrystallized from ethyl acetate-hexane. melting point: 144-145.

### Example 195

To a mixture of {3-cyclohexyl-1-[5-(trifluoromethyl)-2-pyridyl]-1H-pyrazol-4-yl}methanol (400 mg), ethyl 4-(3-hydroxyphenyl)butanoate (260 mg), tributylphosphine (510 mg) and tetrahydrofuran (30 ml) was added 1,1′-azodicarbonyldipiperidine (640 mg) at room temperature and the mixture was stirred overnight. The reaction solution was concentrated and isopropyl ether (20 ml) was added to the residue. The insoluble material was removed by filtration and the filtrate was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and ethanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give 4-(3-{3-cyclohexyl-1-[5-(trifluoromethyl)-2-pyridyl]-1H-pyrazol-4-yl}methoxyphenyl)butanoic acid (390 mg, yield 65%). The crystals were recrystallized from ethyl acetate-hexane. melting point: 128-129° C.

### Example 196

To a mixture of 3-{3-phenyl-1-[5-(trifluoromethyl)-2-pyridyl]-1H-pyrazol-4-yl}-1-propanol (400 mg), methyl (2-hydroxy-3-methoxyphenyl)acetate (240 mg), tributylphosphine (470 mg) and tetrahydrofuran (30 ml) was added 1,1′-azodicarbonyldipiperidine (590 mg) at room temperature and the mixture was stirred overnight. The reaction solution was concentrated and isopropyl ether (20 ml) was added to the residue. The insoluble material was removed by filtration and the filtrate was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give [3-methoxy-2-(3-{3-phenyl-1-[5-(trifluoromethyl)-2-pyridyl]-1H-pyrazol-4-yl}propoxy)phenylacetic acid (290 mg, yield 50%). The crystals were recrystallized from ethyl acetate-hexane. melting point: 134-135° C.

### Example 197

To a mixture of 3-{3-butyl-1-[5-(trifluoromethyl)-2-pyridyl]-1H-pyrazol-4-yl}-1-propanol (400 mg), methyl (2-hydroxy-3-methoxyphenyl)acetate (250 mg), tributylphosphine (510 mg) and tetrahydrofuran (30 ml) was added 1,1′-azodicarbonyldipiperidine (630 mg) at room temperature and the mixture was stirred overnight. The reaction solution was concentrated and isopropyl ether (20 ml) was added to the residue. The insoluble material was removed by filtration and the filtrate was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give [2-(3-{3-butyl-1-[5-(trifluoromethyl)-2-pyridyl]-1H-pyrazol-4-yl}propoxy)-3-methoxyphenyl]acetic acid (420 mg, yield 70%). The crystals were recrystallized from ethyl acetate-hexane. melting point: 121-122° C.

### Example 198

To a mixture of 3-{3-butyl-1-[5-(trifluoromethyl)-2-pyridyl]-1H-pyrazol-4-yl}-1-propanol (400 mg), methyl (2-hydroxyphenyl)acetate (210 mg), tributylphosphine (510 mg) and tetrahydrofuran (30 ml) was added 1,1′-azodicarbonyldipiperidine (630 mg) at room temperature and the mixture was stirred overnight. The reaction solution was concentrated and isopropyl ether (20 ml) was added to the residue. The insoluble material was removed by filtration and the filtrate was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give [2-(3-{3-butyl-1-[5-(trifluoromethyl)-2-pyridyl]-1H-pyrazol-4-yl}propoxy)phenyl]acetic acid (420 mg, yield 75%). The crystals were recrystallized from ethyl acetate-hexane. melting point: 106-107° C.

### Example 199

To a mixture of 3-{3-butyl-1-[5-(trifluoromethyl)-2-pyridyl]-1H-pyrazol-4-yl}-1-propanol (350 mg), methyl (3-hydroxy-1-methyl-1H-pyrazol-4-yl)acetate (190 mg), tributylphosphine (450 mg) and tetrahydrofuran (30 ml) was added 1,1′-azodicarbonyldipiperidine (570 mg) at room temperature and the mixture was stirred overnight. The reaction solution was concentrated and isopropyl ether (20 ml) was added to the residue. The insoluble material was removed by filtration and the filtrate was concentrated. The residue was subjected to silica gel column chromatography, and a colorless oil was obtained from a fraction eluted with ethyl acetate-hexane (1:1, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give [3-(3-{3-butyl-1-[5-(trifluoromethyl)-2-pyridyl]-1H-pyrazol-4-yl}propoxy)-1-methyl-1H-pyrazol-4-yl]acetic acid (420 mg, yield 84%). The crystals were recrystallized from ethyl acetate-hexane. melting point: 125-126° C.

### Example 200

To a mixture of 3-{3-isopropyl-1-[5-(trifluoromethyl)-2-pyridyl]-1H-pyrazol-4-yl}-1-propanol (500 mg), methyl (4-ethoxy-3-hydroxyphenyl)acetate (340 mg), tributylphosphine (650 mg) and tetrahydrofuran (30 ml) was added 1,1′-azodicarbonyldipiperidine (810 mg) at room temperature and the mixture was stirred overnight. The reaction solution was concentrated and isopropyl ether (20 ml) was added to the residue. The insoluble material was removed by filtration and the filtrate was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give [4-ethoxy-3-(3-{3-isopropyl-1-[5-(trifluoromethyl)-2-pyridyl]-1H-pyrazol-4-yl}propoxy)phenyl]acetic acid (580 mg, yield 74%). The crystals were recrystallized from ethyl acetate-hexane. melting point: 116-117° C.

### Example 201

To a mixture of 3-{3-cyclohexyl-1-[5-(trifluoromethyl)-2-pyridyl]-1H-pyrazol-4-yl}-1-propanol (400 mg), methyl (4-ethoxy-3-hydroxyphenyl)acetate (250 mg), tributylphosphine (460 mg) and tetrahydrofuran (30 ml) was added 1,1′-azodicarbonyldipiperidine (580 mg) at room temperature and the mixture was stirred overnight. The reaction solution was concentrated and isopropyl ether (20 ml) was added to the residue. The insoluble material was removed by filtration and the filtrate was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give [3-(3-{3-cyclohexyl-1-[5-(trifluoromethyl)-2-pyridyl]-1H-pyrazol-4-yl}propoxy)-4-ethoxyphenyl]acetic acid (510 mg, yield 85%). The crystals were recrystallized from ethyl acetate-hexane. melting point: 134-135° C.

### Example 202

To a mixture of 3-{3-butyl-1-[5-(trifluoromethyl)-2-pyridyl]-1H-pyrazol-4-yl}-1-propanol (400 mg), methyl (4-ethoxy-3-hydroxyphenyl)acetate (260 mg), tributylphosphine (510 mg) and tetrahydrofuran (30 ml) was added 1,1′-azodicarbonyldipiperidine (630 mg) at room temperature and the mixture was stirred overnight. The reaction solution was concentrated and isopropyl ether (20 ml) was added to the residue. The insoluble material was removed by filtration and the filtrate was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give [3-(3-{3-butyl-1-[5-(trifluoromethyl)-2-pyridyl]-1H-pyrazol-4-yl}propoxy)-4-ethoxyphenyl]acetic acid (510 mg, yield 83%). The crystals were recrystallized from ethyl acetate-hexane. melting point: 105-106° C.

### Example 203

To a mixture of 3-{3-isopropyl-1-[5-(trifluoromethyl)-2-pyridyl]-1H-pyrazol-4-yl}-1-propanol (400 mg), methyl (5-hydroxy-3-methoxyphenyl)acetate (260 mg), tributylphosphine (550 mg) and tetrahydrofuran (30 ml) was added 1,1′-azodicarbonyldipiperidine (690 mg) at room temperature and the mixture was stirred overnight. The reaction solution was concentrated and isopropyl ether (20 ml) was added to the residue. The insoluble material was removed by filtration and the filtrate was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give [3-(3-{3-isopropyl-1-[5-(trifluoromethyl)-2-pyridyl]-1H-pyrazol-4-yl}propoxy)-5-methoxyphenyl]acetic acid (530 mg, yield 87%). The crystals were recrystallized from ethyl acetate-hexane. melting point: 109-110° C.

### Example 204

To a mixture of 3-{3-propyl-1-[5-(trifluoromethyl)-2-pyridyl]-1H-pyrazol-4-yl}-1-propanol (400 mg), methyl (4-ethoxy-3-hydroxyphenyl)acetate (270 mg), tributylphosphine (550 mg) and tetrahydrofuran (30 ml) was added 1,1′-azodicarbonyldipiperidine (690 mg) at room temperature and the mixture was stirred overnight. The reaction solution was concentrated and isopropyl ether (20 ml) was added to the residue. The insoluble material was removed by filtration and the filtrate was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give [4-ethoxy-3-(3-{3-propyl-1-[5-(trifluoromethyl)-2-pyridyl]-1H-pyrazol-4-yl}propoxy)phenyl]acetic acid (540 mg, yield 86%). The crystals were recrystallized from ethyl acetate-hexane. melting point: 124-125° C.

### Example 205

To a mixture of 3-{3-isopropyl-1-[5-(trifluoromethyl)-2-pyridyl]-1H-pyrazol-4-yl}-1-propanol (400 mg), methyl (2-hydroxy-4-methoxyphenyl)acetate (260 mg), tributylphosphine (550 mg) and tetrahydrofuran (30 ml) was added 1,1′-azodicarbonyldipiperidine (690 mg) at room temperature and the mixture was stirred overnight. The reaction solution was concentrated and isopropyl ether (20 ml) was added to the residue. The insoluble material was removed by filtration and the filtrate was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give [2-(3-{3-isopropyl-1-[5-(trifluoromethyl)-2-pyridyl]-1H-pyrazol-4-yl}propoxy)-4-methoxyphenyl]acetic acid (460 mg, yield 75%). The crystals were recrystallized from ethyl acetate-hexane. melting point: 143-144° C.

### Example 206

To a mixture of 3-{3-isopropyl-1-[5-(trifluoromethyl)-2-pyridyl]-1H-pyrazol-4-yl}-1-propanol (400 mg), methyl (2-hydroxy-5-methoxyphenyl)acetate (270 mg), tributylphosphine (550 mg) and tetrahydrofuran (30 ml) was added 1,1′-azodicarbonyldipiperidine (690 mg) at room temperature and the mixture was stirred overnight. The reaction solution was concentrated and isopropyl ether (20 ml) was added to the residue. The insoluble material was removed by filtration and the filtrate was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give [2-(3-{3-isopropyl-1-[5-(trifluoromethyl)-2-pyridyl]-1H-pyrazol-4-yl}propoxy)-5-methoxyphenyl]acetic acid (460 mg, yield 75%). The crystals were recrystallized from ethyl acetate-hexane. melting point: 143-144° C.

### Example 207

To a mixture of 3-{3-isopropyl-1-[5-(trifluoromethyl)-2-pyridyl]-1H-pyrazol-4-yl}-1-propanol (400 mg), methyl (3-fluoro-2-hydroxyphenyl)acetate (240 mg), tributylphosphine (550 mg) and tetrahydrofuran (30 ml) was added 1,1′-azodicarbonyldipiperidine (690 mg) at room temperature and the mixture was stirred overnight. The reaction solution was concentrated and isopropyl ether (20 ml) was added to the residue. The insoluble material was removed by filtration and the filtrate was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give [3-fluoro-2-(3-{3-isopropyl-1-[5-(trifluoromethyl)-2-pyridyl]-1H-pyrazol-4-yl}propoxy)phenyl]acetic acid (560 mg, yield 94%). The crystals were recrystallized from ethyl acetate-hexane. melting point: 140-141° C.

### Example 208

To a mixture of 3-{3-isopropyl-1-[5-(trifluoromethyl)-2-pyridyl]-1H-pyrazol-4-yl}-1-propanol (400 mg), methyl (6-hydroxy-2-methoxyphenyl)acetate (260 mg), tributylphosphine (550 mg) and tetrahydrofuran (30 ml) was added 1,1′-azodicarbonyldipiperidine (690 mg) at room temperature and the mixture was stirred overnight. The reaction solution was concentrated and isopropyl ether (20 ml) was added to the residue. The insoluble material was removed by filtration and the filtrate was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give [2-(3-{3-isopropyl-1-[5-(trifluoromethyl)-2-pyridyl]-1H-pyrazol-4-yl}propoxy)-6-methoxyphenyl]acetic acid (460 mg, yield 75%). The crystals were recrystallized from ethyl acetate-hexane. melting point: 183-184° C.

### Example 209

To a mixture of 3-{3-isopropyl-1-[5-(trifluoromethyl)-2-pyridyl]-1H-pyrazol-4-yl}-1-propanol (400 mg), methyl 5-hydroxy-2-methoxyphenylacetate (260 mg), tributylphosphine (550 mg) and tetrahydrofuran (30 ml) was added 1,1′-azodicarbonyldipiperidine (690 mg) at room temperature and the mixture was stirred overnight. The reaction solution was concentrated and isopropyl ether (20 ml) was added to the residue. The insoluble material was removed by filtration and the filtrate was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give [5-(3-{3-isopropyl-1-[5-(trifluoromethyl)-2-pyridyl]-1H-pyrazol-4-yl}propoxy)}-2-methoxyphenyl]acetic acid (510 mg, yield 84%). The crystals were recrystallized from ethyl acetate-hexane. melting point: 127-128° C.

### Example 210

To a mixture of 3-{3-cyclohexyl-1-[5-(trifluoromethyl)-2-pyridyl]-1H-pyrazol-4-yl}-1-propanol (400 mg), methyl (3-fluoro-2-hydroxyphenyl)acetate (220 mg), tributylphosphine (460 mg) and tetrahydrofuran (30 ml) was added 1,1′-azodicarbonyldipiperidine (580 mg) at room temperature and the mixture was stirred overnight. The reaction solution was concentrated and isopropyl ether (20 ml) was added to the residue. The insoluble material was removed by filtration and the filtrate was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give [2-(3-{3-cyclohexyl-1-[5-(trifluoromethyl)-2-pyridyl]-1H-pyrazol-4-yl}propoxy)-3-fluorophenyl]acetic acid (520 mg, yield 91%). The crystals were recrystallized from ethyl acetate-hexane. melting point: 149-150° C.

### Example 211

To a mixture of 3-{3-propyl-1-[5-(trifluoromethyl)-2-pyridyl]-1H-pyrazol-4-yl}-1-propanol (400 mg), methyl (3-fluoro-2-hydroxyphenyl)acetate (240 mg), tributylphosphine (550 mg) and tetrahydrofuran (30 ml) was added 1,1′-azodicarbonyldipiperidine (690 mg) at room temperature and the mixture was stirred overnight. The reaction solution was concentrated and isopropyl ether (20 ml) was added to the residue. The insoluble material was removed by filtration and the filtrate was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give [3-fluoro-2-(3-{3-propyl-1-[5-(trifluoromethyl)-2-pyridyl]-1H-pyrazol-4-yl}propoxy)phenyl]acetic acid (510 mg, yield 86%). The crystals were recrystallized from ethyl acetate-hexane. melting point: 105-106° C.

### Example 212

To a mixture of 3-{3-(1-ethylpropyl)-1-[5-(trifluoromethyl)-2-pyridyl]-1H-pyrazol-4-yl}-1-propanol (400 mg), methyl (2-hydroxy-3-methoxyphenyl)acetate (240 mg), tributylphosphine (480 mg) and tetrahydrofuran (30 ml) was added 1,1′-azodicarbonyldipiperidine (600 mg) at room temperature and the mixture was stirred overnight. The reaction solution was concentrated and isopropyl ether (20 ml) was added to the residue. The insoluble material was removed by filtration and the filtrate was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give [2-(3-{3-(1-ethylpropyl)-1-[5-(trifluoromethyl)-2-pyridyl]-1H-pyrazol-4-yl}propoxy)-3-methoxyphenyl]acetic acid (510 mg, yield 86%). The crystals were recrystallized from ethyl acetate-hexane. melting point: 100-101° C.

### Example 213

To a mixture of 3-{3-(1-ethylpropyl)-1-[5-(trifluoromethyl)-2-pyridyl]-1H-pyrazol-4-yl}-1-propanol (400 mg), methyl (2-hydroxy-3-methylphenyl)acetate (220 mg), tributylphosphine (480 mg) and tetrahydrofuran (30 ml) was added 1,1′-azodicarbonyldipiperidine (600 mg) at room temperature and the mixture was stirred overnight at 50° C. The reaction solution was concentrated and isopropyl ether (20 ml) was added to the residue. The insoluble material was removed by filtration and the filtrate was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give [2-(3-{3-(1-ethylpropyl)-1-[5-(trifluoromethyl)-2-pyridyl]-1H-pyrazol-4-yl}propoxy)-3-methylphenyl]acetic acid (430 mg, yield 75%). The crystals were recrystallized from ethyl acetate-hexane. melting point: 125-126° C.

### Example 214

To a mixture of 3-{3-(1-ethylpropyl)-1-[5-(trifluoromethyl)-2-pyridyl]-1H-pyrazol-4-yl}-1-propanol (400 mg), methyl (3-fluoro-2-hydroxyphenyl)acetate (220 mg), tributylphosphine (480 mg) and tetrahydrofuran (30 ml) was added 1,1′-azodicarbonyldipiperidine (600 mg) at room temperature and the mixture was stirred overnight. The reaction solution was concentrated and isopropyl ether (20 ml) was added to the residue. The insoluble material was removed by filtration and the filtrate was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give [2-(3-{3-(1-ethylpropyl)-1-[5-(trifluoromethyl)-2-pyridyl]-1H-pyrazol-4-yl}propoxy)-3-fluorophenyl]acetic acid (390 mg, yield 60%). The crystals were recrystallized from ethyl acetate-hexane. melting point: 102-103° C.

### Example 215

To a mixture of 3-{3-(1-ethylpropyl)-1-[5-(trifluoromethyl)-2-pyridyl]-1H-pyrazol-4-yl}-1-propanol (400 mg), methyl (2-hydroxyphenyl)acetate (200 mg), tributylphosphine (480 mg) and tetrahydrofuran (30 ml) was added 1,1′-azodicarbonyldipiperidine (600 mg) at room temperature and the mixture was stirred overnight. The reaction solution was concentrated and isopropyl ether (20 ml) was added to the residue. The insoluble material was removed by filtration and the filtrate was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give [2-(3-{3-(1-ethylpropyl)-1-[5-(trifluoromethyl)-2-pyridyl]-1H-pyrazol-4-yl}propoxy)phenyl]acetic acid (370 mg, yield 66%). The crystals were recrystallized from ethyl acetate-hexane. melting point: 65-66° C.

### Example 216

To a mixture of 3-{3-(1-methylpropyl)-1-[5-(trifluoromethyl)-2-pyridyl]-1H-pyrazol-4-yl}-1-propanol (400 mg), methyl (2-hydroxy-3-methoxyphenyl)acetate (250 mg), tributylphosphine (500 mg) and tetrahydrofuran (30 ml) was added 1,1′-azodicarbonyldipiperidine (630 mg) at room temperature and the mixture was stirred overnight. The reaction solution was concentrated and isopropyl ether (20 ml) was added to the residue. The insoluble material was removed by filtration and the filtrate was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give [3-methoxy-2-(3-{3-(1-methylpropyl)-1-[5-(trifluoromethyl)-2-pyridyl]-1H-pyrazol-4-yl}propoxy)phenyl]acetic acid (510 mg, yield 85%). The crystals were recrystallized from ethyl acetate-hexane. melting point: 117-118° C.

### Example 217

To a mixture of 3-{1-[3-chloro-5-(trifluoromethyl)-2-pyridyl]-3-isopropyl-1H-pyrazol-4-yl}-1-propanol (500 mg), methyl (2-hydroxy-3-methoxyphenyl)acetate (300 mg), tributylphosphine (600 mg) and tetrahydrofuran (30 ml) was added 1,1′-azodicarbonyldipiperidine (750 mg) at room temperature and the mixture was stirred overnight. The reaction solution was concentrated and isopropyl ether (20 ml) was added to the residue. The insoluble material was removed by filtration and the filtrate was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give [2-(3-{1-[3-chloro-5-(trifluoromethyl)-2-pyridyl]-3-isopropyl-1H-pyrazol-4-yl}propoxy)-3-methoxyphenyl]acetic acid (610 mg, yield 83%). The crystals were recrystallized from ethyl acetate-hexane. melting point: 96-97° C.

### Example 218

To a mixture of 3-[1-(5-bromo-2-pyridyl)-3-isopropyl-1H-pyrazol-4-yl]-1-propanol (500 mg), methyl (2-hydroxy-3-methoxyphenyl)acetate (310 mg), tributylphosphine (630 mg) and tetrahydrofuran (30 ml) was added 1,1′-azodicarbonyldipiperidine (790 mg) at room temperature and the mixture was stirred overnight. The reaction solution was concentrated and isopropyl ether (20 ml) was added to the residue. The insoluble material was removed by filtration and the filtrate was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give (2-{3-[1-(5-bromo-2-pyridyl)-3-isopropyl-1H-pyrazol-4-yl]propoxy}-3-methoxyphenyl)acetic acid (720 mg, yield 95%). The crystals were recrystallized from ethyl acetate-hexane. melting point: 152-153° C.

### Example 219

To a mixture of 3-{3-(1-methylpropyl)-1-[5-(trifluoromethyl)-2-pyridyl]-1H-pyrazol-4-yl}-1-propanol (400 mg), methyl (2-hydroxy-3-methylphenyl)acetate (230 mg), tributylphosphine (500 mg) and tetrahydrofuran (30 ml) was added 1,1′-azodicarbonyldipiperidine (630 mg) at room temperature and the mixture was stirred overnight at 50° C. The reaction solution was concentrated and isopropyl ether (20 ml) was added to the residue. The insoluble material was removed by filtration and the filtrate was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give [3-methyl-2-(3-{3-(1-methylpropyl)-1-[5-(trifluoromethyl)-2-pyridyl]-1H-pyrazol-4-yl}propoxy)phenyl]acetic acid (380 mg, yield 66%). The crystals were recrystallized from ethyl acetate-hexane. melting point: 134-135° C.

### Example 220

To a mixture of 3-{3-(1-methylbutyl)-1-[5-(trifluoromethyl)-2-pyridyl]-1H-pyrazol-4-yl}-1-propanol (400 mg), methyl (2-hydroxy-3-methoxyphenyl)acetate (240 mg), tributylphosphine (480 mg) and tetrahydrofuran (30 ml) was added 1,1′-azodicarbonyldipiperidine (600 mg) at room temperature and the mixture was stirred overnight. The reaction solution was concentrated and isopropyl ether (20 ml) was added to the residue. The insoluble material was removed by filtration and the filtrate was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours. 1N Hydrochloric acid (5 ml) was added and the mixture was extracted with ethyl acetate. The ethyl acetate layer was washed with saturated aqueous sodium chloride solution, dried (MgSO₄) and concentrated. The obtained colorless crystals were collected by filtration to give [3-methoxy-2-(3-{3-(1-methylbutyl)-1-[5-(trifluoromethyl)-2-pyridyl]-1H-pyrazol-4-yl}propoxy)phenyl]acetic acid (480 mg, yield 81%). The crystals were recrystallized from ethyl acetate-hexane. melting point: 107-108° C.

### Example 221

To a mixture of 3-{3-(1-ethylpropyl)-1-[5-(trifluoromethyl)-2-pyridyl]-1H-pyrazol-4-yl}-1-propanol (400 mg), methyl (3-ethoxy-2-hydroxyphenyl)acetate (250 mg), tributylphosphine (480 mg) and tetrahydrofuran (30 ml) was added 1,1′-azodicarbonyldipiperidine (600 mg) at room temperature and the mixture was stirred overnight. The reaction solution was concentrated and isopropyl ether (20 ml) was added to the residue. The insoluble material was removed by filtration and the filtrate was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours. 1N Hydrochloric acid (5 ml) was added and the mixture was extracted with ethyl acetate. The ethyl acetate layer was washed with saturated aqueous sodium chloride solution, dried (MgSO₄) and concentrated. The obtained colorless crystals were collected by filtration to give [3-ethoxy-2-(3-{3-(1-ethylpropyl)-1-[5-(trifluoromethyl)-2-pyridyl]-1H-pyrazol-4-yl}propoxy)phenyl]acetic acid (540 mg, yield 89%). The crystals were recrystallized from ethyl acetate-hexane. melting point: 83-84° C.

### Example 222

To a mixture of 3-{3-(1-methylbutyl)-1-[5-(trifluoromethyl)-2-pyridyl]-1H-pyrazol-4-yl}-1-propanol (400 mg), methyl (2-hydroxy-3-methylphenyl)acetate (210 mg), tributylphosphine (480 mg) and tetrahydrofuran (30 ml) was added 1,1′-azodicarbonyldipiperidine (600 mg) at room temperature and the mixture was stirred overnight at 50° C. The reaction solution was concentrated and isopropyl ether (20 ml) was added to the residue. The insoluble material was removed by filtration and the filtrate was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours. 1N Hydrochloric acid (5 ml) was added and the mixture was extracted with ethyl acetate. The ethyl acetate layer was washed with saturated aqueous sodium chloride solution, dried (MgSO₄) and concentrated. The obtained colorless crystals were collected by filtration to give [3-methyl-2-(3-{3-(1-methylbutyl)-1-[5-(trifluoromethyl)-2-pyridyl]-1H-pyrazol-4-yl}propoxy)phenyl]acetic acid (360 mg, yield 63%). The crystals were recrystallized from ethyl acetate-hexane. melting point: 147-148° C.

### Example 223

To a mixture of 3-[1-(5-chloro-2-pyridyl)-3-(1-ethylpropyl)-1H-pyrazol-4-yl]-1-propanol (670 mg), methyl (2-hydroxy-3-methoxyphenyl)acetate (430 mg), tributylphosphine (910 mg) and tetrahydrofuran (40 ml) was added 1,1′-azodicarbonyldipiperidine (1.15 g) at room temperature and the mixture was stirred overnight. The reaction solution was concentrated and isopropyl ether (40 ml) was added to the residue. The insoluble material was removed by filtration and the filtrate was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours. 1N Hydrochloric acid (5 ml) was added and the mixture was extracted with ethyl acetate. The ethyl acetate layer was washed with saturated aqueous sodium chloride solution, dried (MgSO₄) and concentrated. The obtained colorless crystals were collected by filtration to give (2-{3-[1-(5-chloro-2-pyridyl)-3-(1-ethylpropyl)-1H-pyrazol-4-yl]propoxy}-3-methoxyphenyl) acetic acid (760 mg, yield 74%). The crystals were recrystallized from ethyl acetate-hexane. melting point: 85-86° C.

### Example 224

To a mixture of 3-[1-(5-chloro-2-pyridyl)-3-(1-ethylpropyl)-1H-pyrazol-4-yl]-1-propanol (500 mg), methyl (2-hydroxy-3-methylphenyl)acetate (300 mg), tributylphosphine (660 mg) and tetrahydrofuran (30 ml) was added 1,1′-azodicarbonyldipiperidine (820 mg) at room temperature and the mixture was stirred overnight at 50° C. The reaction solution was concentrated and isopropyl ether (20 ml) was added to the residue. The insoluble material was removed by filtration and the filtrate was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours. 1N Hydrochloric acid (5 ml) was added and the mixture was extracted with ethyl acetate. The ethyl acetate layer was washed with saturated aqueous sodium chloride solution, dried (MgSO₄) and concentrated. The obtained colorless crystals were collected by filtration to give (2-{3-[1-(5-chloro-2-pyridyl)-3-(1-ethylpropyl)-1H-pyrazol-4-yl]propoxy}-3-methylphenyl)acetic acid (280 mg, yield 46%). The crystals were recrystallized from ethyl acetate-hexane. melting point: 106-107° C.

### Example 225

To a mixture of 3-[1-(5-chloro-2-pyridyl)-3-(1-ethylpropyl)-1H-pyrazol-4-yl]-1-propanol (500 mg), methyl (3-ethoxy-2-hydroxyphenyl)acetate (350 mg), tributylphosphine (660 mg) and tetrahydrofuran (30 ml) was added 1,1′-azodicarbonyldipiperidine (820 mg) at room temperature and the mixture was stirred overnight. The reaction solution was concentrated and isopropyl ether (20 ml) was added to the residue. The insoluble material was removed by filtration and the filtrate was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (5 ml), tetrahydrofuran (5 ml) and methanol (5 ml) was stirred at room temperature for 5 hours. 1N Hydrochloric acid (5 ml) was added and the mixture was extracted with ethyl acetate. The ethyl acetate layer was washed with saturated aqueous sodium chloride solution, dried (MgSO₄) and concentrated. The obtained colorless crystals were collected by filtration to give (2-{3-[1-(5-chloro-2-pyridyl)-3-(1-ethylpropyl)-1H-pyrazol-4-yl]propoxy}-3-ethoxyphenyl)acetic acid (590 mg, yield 75%). The crystals were recrystallized from ethyl acetate-hexane. melting point: 92-93° C.

### Example 226

To a mixture of 3-{3-isopropyl-1-[5-(trifluoromethyl)-2-pyridinyl]-1H-pyrazol-4-yl}-1-propanol (380 mg), methyl (3-ethyl-2-hydroxyphenyl)acetate (240 mg), tributylphosphine (520 mg) and tetrahydrofuran (30 ml) was added 1,1′-azodicarbonyldipiperidine (650 mg) at room temperature and the mixture was stirred overnight at 65° C. The reaction solution was concentrated and isopropyl ether was added. The insoluble material was removed by filtration and the filtrate was concentrated. Then, the residue was subjected to silica gel column chromatography, and was obtained from a fraction eluted with ethyl acetate-hexane (1:9, volume ratio), methyl [3-ethyl-2-(3-{3-isopropyl-1-[5-(trifluoromethyl)-2-pyridinyl]-1H-pyrazol-4-yl}propoxy)phenyl]acetate (400 mg, yield 68%) as a colorless oil.

¹H-NMR (CDCl₃)δ: 1.23 (3H, t, J=6.8 Hz), 1.35 (6H, d, J=6.8 Hz), 2.12-2.17 (2H, m), 2.67 (2H, q, J=6.8 Hz), 2.74 (2H, t, J=8.4 Hz), 3.00-3.10 (1H,m), 3.68 (2H, s), 3.68 (3H, s), 3.87 (2H, t, J=6.4 Hz), 7.04 (1H, t, J=7.6 Hz), 7.10 (1H, dd, J=7.6, 2.0 Hz), 7.15 (1H, dd, J=7.6,2.0 Hz), 7.94-7.97 (1H, m), 8.04 (1H, d, J=8.8 Hz), 8.33 (1H, s), 8.60-8.61 (1H, m).

### Example 227

A mixture of methyl [3-ethyl-2-(3-{3-isopropyl-1-[5-(trifluoromethyl)-2-pyridinyl]-1H-pyrazol-4-yl}propoxy)phenyl]acetate (400 mg), 1N aqueous sodium hydroxide solution (5 ml), tetrahydrofuran (5 ml) and methanol (5 ml) was stirred at room temperature for 4 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give [3-ethyl-2-(3-{3-isopropyl-1-[5-(trifluoromethyl)-2-pyridinyl]-1H-pyrazol-4-yl}propoxy)phenyl]acetic acid (370 mg, yield 96%). The crystals were recrystallized from hexane-ethyl acetate. melting point: 155-156° C.

### Example 228

To a mixture of 3-{3-propyl-1-[5-(trifluoromethyl)-2-pyridinyl]-1H-pyrazol-4-yl}-1-propanol (380 mg), methyl (3-ethyl-2-hydroxyphenyl)acetate (240 mg), tributylphosphine (520 mg) and tetrahydrofuran (30 ml) was added 1,1′-azodicarbonyldipiperidine (650 mg) at room temperature and the mixture was stirred overnight at 65° C. The reaction solution was concentrated and isopropyl ether was added. The insoluble material was removed by filtration and the filtrate was concentrated. Then, the residue was subjected to silica gel column chromatography, and methyl [3-ethyl-2-(3-{3-propyl-1-[5-(trifluoromethyl)-2-pyridinyl]-1H-pyrazol-4-yl}propoxy)phenyl]acetate (490 mg, yield 83%) was obtained as a colorless oil from a fraction eluted with ethyl acetate-hexane (1:9, volume ratio).

¹H-NMR (CDCl₃)δ: 1.03 (3H, t, J=7.2 Hz), 1.23 (3H, t, J=7.6 Hz), 1.73-1.80 (2H, m), 2.11-2.17 (2H, m), 2.64-2.73 (6H,m), 3.68 (2H, s), 3.68 (3H, s), 3.86 (2H, t, J=6.4 Hz), 7.04 (1H, t, J=7.6 Hz), 7.11 (1H, dd, J=7.6, 2.0 Hz), 7.15 (1H, dd, J=7.6, 2.0 Hz), 7.96 (1H, dd, J=8.8, 2.0 Hz), 8.02 (1H, d, J=8.8 Hz), 8.34 (1H, s), 8.61 (1H, m).

### Example 229

A mixture of methyl [3-ethyl-2-(3-{3-propyl-1-[5-(trifluoromethyl)-2-pyridinyl]-1H-pyrazol-4-yl}propoxy)phenyl]acetate (490 mg), 1N aqueous sodium hydroxide solution (5 ml), tetrahydrofuran (5 ml) and methanol (5 ml) was stirred at room temperature for 4 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give [3-ethyl-2-(3-{3-propyl-1-[5-(trifluoromethyl)-2-pyridinyl]-1H-pyrazol-4-yl}propoxy)phenyl]acetic acid (450 mg, yield 96%). The crystals were recrystallized from hexane-ethyl acetate. melting point: 146-147° C.

### Example 230

To a mixture of 3-{3-isopropyl-1-[5-(trifluoromethyl)-2-pyridinyl]-1H-pyrazol-4-yl}-1-propanol (570 mg), methyl (3-cyano-2-hydroxyphenyl)acetate (360 mg), tributylphosphine (790 mg) and tetrahydrofuran (40 ml) was added 1,1′-azodicarbonyldipiperidine (980 mg) at room temperature and the mixture was stirred overnight at 65° C. The reaction solution was concentrated and isopropyl ether was added. The insoluble material was removed by filtration and the filtrate was concentrated. Then, the residue was subjected to silica gel column chromatography, and methyl [3-cyano-2-(3-{3-isopropyl-1-[5-(trifluoromethyl)-2-pyridinyl]-1H-pyrazol-4-yl}propoxy)phenyl]acetate (680 mg, yield 75%) was obtained as a colorless oil from a fraction eluted with ethyl acetate-hexane (1:9, volume ratio).

¹H-NMR (CDCl₃)δ: 1.35 (6H, d, J=7.2 Hz), 2.14-2.21 (2H, m), 2.72-2.76 (2H, m), 3.03-3.09 (1H,m), 3.69 (2H, s), 3.69 (3H, s), 4.32 (2H, t, J=6.0 Hz), 7.13 (1H, t, J=7.6 Hz), 7.48 (1H, dd, J=7.6, 1.6 Hz), 7.54 (1H, dd, J=7.6, 1.6 Hz), 7.95 (1H, dd, J=8.4, 2.8 Hz), 8.04 (1H, d, J=8.4 Hz), 8.33 (1H, s), 8.60-8.61 (1H, m).

### Example 231

A mixture of methyl [3-cyano-2-(3-{3-isopropyl-1-[5-(trifluoromethyl)-2-pyridinyl]-1H-pyrazol-4-yl}propoxy)phenyl]acetate (650 mg), 1N aqueous sodium hydroxide solution (5 ml), tetrahydrofuran (5 ml) and methanol (5 ml) was stirred at room temperature for 4 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give [3-cyano-2-(3-{3-isopropyl-1-[5-(trifluoromethyl)-2-pyridinyl]-1H-pyrazol-4-yl}propoxy)phenyl]acetic acid (590 mg, yield 95%). The crystals were recrystallized from hexane-ethyl acetate. melting point: 144-145° C.

### Example 232

To a mixture of 3-{3-propyl-1-[5-(trifluoromethyl)-2-pyridinyl]-1H-pyrazol-4-yl}-1-propanol (320 mg), methyl (3-cyano-2-hydroxyphenyl)acetate (200 mg), tributylphosphine (440 mg) and tetrahydrofuran (30 ml) was added 1,1′-azodicarbonyldipiperidine (550 mg) at room temperature and the mixture was stirred overnight at 65° C. The reaction solution was concentrated and isopropyl ether was added. The insoluble material was removed by filtration and the filtrate was concentrated. Then, the residue was subjected to silica gel column chromatography, and methyl [3-cyano-2-(3-{3-propyl-1-[5-(trifluoromethyl)-2-pyridinyl]-1H-pyrazol-4-yl}propoxy)phenyl]acetate (360 mg, yield 71%) was obtained as a colorless oil from a fraction eluted with ethyl acetate-hexane (1:9, volume ratio).

¹H-NMR (CDCl₃)δ: 1.03 (3H, t, J=7.6 Hz), 1.72-1.80 (2H, m), 2.12-2.19 (2H, m), 2.65 (2H,t, J=8.0 Hz), 2.72 (2H,t, J=8.4 Hz), 3.69 (2H, s), 3.70 (3H, s), 4.31 (2H, t, J=6.0 Hz), 7.13 (1H, t, J=8.0 Hz), 7.48 (1H, dd, J=8.0, 1.6 Hz), 7.53 (1H, dd, J=8.0, 1.6 Hz), 7.96 (1H, dd, J=8.4, 2.0 Hz), 8.02 (1H, d, J=8.4 Hz), 8.34 (1H, s), 8.61-8.62 (1H, m).

### Example 233

A mixture of methyl [3-cyano-2-(3-{3-propyl-1-[5-(trifluoromethyl)-2-pyridinyl]-1H-pyrazol-4-yl}propoxy)phenyl]acetate (330 mg), 1N aqueous sodium hydroxide solution (5 ml), tetrahydrofuran (5 ml) and methanol (5 ml) was stirred at room temperature for 4 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give [3-cyano-2-(3-{3-propyl-1-[5-(trifluoromethyl)-2-pyridinyl]-1H-pyrazol-4-yl}propoxy)phenyl]acetic acid (270 mg, yield 86%). The crystals were recrystallized from hexane-ethyl acetate. melting point: 146-147° C.

### Example 234

To a mixture of 3-{3-isopropyl-1-[5-(trifluoromethyl)-2-pyridinyl]-1H-pyrazol-4-yl}-1-propanol (570 mg), methyl (3-bromo-2-hydroxyphenyl)acetate (460 mg), tributylphosphine (790 mg) and tetrahydrofuran (40 ml) was added 1,1′-azodicarbonyldipiperidine (980 mg) at room temperature and the mixture was stirred overnight at 65° C. The reaction solution was concentrated and isopropyl ether was added. The insoluble material was removed by filtration and the filtrate was concentrated. Then, the residue was subjected to silica gel column chromatography, and methyl [3-bromo-2-(3-{3-isopropyl-1-[5-(trifluoromethyl)-2-pyridinyl]-1H-pyrazol-4-yl}propoxy)phenyl]acetate (750 mg, yield 76%) was obtained as a colorless oil from a fraction eluted with ethyl acetate-hexane (1:9, volume ratio).

¹H-NMR (CDCl₃)δ: 1.35 (6H, d, J=6.8 Hz), 2.14-2.21 (2H, m), 2.76 (2H, t, J=8.4 Hz), 2.60-3.10 (1H,m), 3.69 (3H, s), 3.71 (2H, s), 4.04 (2H, t, J=6.4 Hz), 6.97 (1H, t, J=8.0 Hz), 7.22 (1H, dd, J=8.0, 1.6 Hz), 7.48 (1H, dd, J=8.0, 1.6 Hz), 7.95 (1H, dd, J=8.8, 2.0 Hz), 8.04 (1H, d, J=8.8 Hz), 8.34 (1H, s), 8.60-8.61 (1H, m).

### Example 235

A mixture of methyl [3-bromo-2-(3-{3-isopropyl-1-[5-(trifluoromethyl)-2-pyridinyl]-1H-pyrazol-4-yl}propoxy)phenyl]acetate (700 mg), 1N aqueous sodium hydroxide solution (5 ml), tetrahydrofuran (5 ml) and methanol (5 ml) was stirred at room temperature for 4 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give [3-bromo-2-(3-{3-isopropyl-1-[5-(trifluoromethyl)-2-pyridinyl]-1H-pyrazol-4-yl}propoxy)phenyl]acetic acid (610 mg, yield 89%). The crystals were recrystallized from hexane-ethyl acetate. melting point: 146-147° C.

### Example 236

To a mixture of 3-{3-propyl-1-[5-(trifluoromethyl)-2-pyridinyl]-1H-pyrazol-4-yl}-1-propanol (430 mg), methyl (3-bromo-2-hydroxyphenyl)acetate (340 mg), tributylphosphine (590 mg) and tetrahydrofuran (40 ml) was added 1,1′-azodicarbonyldipiperidine (730 mg) at room temperature and the mixture was stirred overnight at 65° C. The reaction solution was concentrated and isopropyl ether was added. The insoluble material was removed by filtration and the filtrate was concentrated. Then, the residue was subjected to silica gel column chromatography, and methyl [3-bromo-2-(3-{3-propyl-1-[5-(trifluoromethyl)-2-pyridinyl]-1H-pyrazol-4-yl}propoxy)phenyl]acetate (570 mg, yield 78%) was obtained as a colorless oil from a fraction eluted with ethyl acetate-hexane (1:9, volume ratio).

¹H-NMR (CDCl₃)δ: 1.03 (3H, t, J=7.6 Hz), 1.72-1.80 (2H, m), 2.12-2.20 (2H, m), 2.66 (2H,t, J=8.0 Hz), 2.73 (2H,t, J=8.4 Hz), 3.69 (3H, s), 3.70 (2H, s), 4.03 (2H, t, J=6.4 Hz), 6.97 (1H, t, J=7.6 Hz), 7.22 (1H, dd, J=7.6, 1.6 Hz), 7.48 (1H, dd, J=7.6, 1.6 Hz), 7.96 (1H, dd, J=8.8, 2.0 Hz), 8.02 (1H, d, J=8.8 Hz), 8.34 (1H, s), 8.61-8.62 (1H, m).

### Example 237

A mixture of methyl [3-bromo-2-(3-{3-propyl-1-[5-(trifluoromethyl)-2-pyridinyl]-1H-pyrazol-4-yl}propoxy)phenyl]acetate (500 mg), 1N aqueous sodium hydroxide solution (5 ml), tetrahydrofuran (5 ml) and methanol (5 ml) was stirred at room temperature 4 hours and 1N hydrochloric acid (5 ml) was added. The mixture was extracted with ethyl acetate. The ethyl acetate layer was washed with saturated aqueous sodium chloride solution, dried (MgSO₄) and concentrated. The obtained colorless crystals were collected by filtration to give [3-bromo-2-(3-{3-propyl-1-[5-(trifluoromethyl)-2-pyridinyl]-1H-pyrazol-4-yl}propoxy)phenyl]acetic acid (380 mg, yield 78%). The crystals were recrystallized from hexane-ethyl acetate. melting point: 145-146° C.

### Example 238

To a mixture of 3-{3-isopropyl-1-[5-(trifluoromethyl)-2-pyridinyl]-1H-pyrazol-4-yl}-1-propanol (430 mg), methyl (3-chloro-2-hydroxyphenyl)acetate (280 mg), tributylphosphine (580 mg) and tetrahydrofuran (30 ml) was added 1,1′-azodicarbonyldipiperidine (730 mg) at room temperature and the mixture was stirred overnight at 65° C. The reaction solution was concentrated and isopropyl ether was added. The insoluble material was removed by filtration and the filtrate was concentrated. Then, the residue was subjected to silica gel column chromatography, and methyl [3-chloro-2-(3-{3-isopropyl-1-[5-(trifluoromethyl)-2-pyridinyl]-1H-pyrazol-4-yl}propoxy)phenyl]acetate (590 mg, yield 85%) was obtained as a colorless oil from a fraction eluted with ethyl acetate-hexane (1:9, volume ratio).

¹H-NMR (CDCl₃)δ: 1.35 (6H, d, J=6.8 Hz), 2.11-2.30 (2H, m), 2.74 (2H, t, J=8.0 Hz), 3.04-3.10 (1H,m), 3.69 (3H, s), 3.70 (2H, s), 4.06 (2H, t, J=6.4 Hz), 7.02 (1H, t, J=8.0 Hz), 7.17 (1H, dd, J=8.0, 1.6 Hz), 7.31 (1H, dd, J=8.0, 1.6 Hz), 7.95 (1H, dd, J=8.8, 2.0 Hz), 8.04 (1H, d, J=8.8 Hz), 8.33 (1H, s), 8.60-8.61 (1H, m).

### Example 239

A mixture of methyl [3-chloro-2-(3-{3-isopropyl-1-[5-(trifluoromethyl)-2-pyridinyl]-1H-pyrazol-4-yl}propoxy)phenyl]acetate (510 mg), 1N aqueous sodium hydroxide solution (5 ml), tetrahydrofuran (5 ml) and methanol (5 ml) was stirred at room temperature for 4 hours. 1N Hydrochloric acid (5 ml) was added and the mixture was extracted with ethyl acetate. The ethyl acetate layer was washed with saturated aqueous sodium chloride solution, dried (MgSO₄) and concentrated. The obtained colorless crystals were collected by filtration to give [3-chloro-2-(3-{3-isopropyl-1-[5-(trifluoromethyl)-2-pyridinyl]-1H-pyrazol-4-yl}propoxy)phenyl]acetic acid (400 mg, yield 81%). The crystals were recrystallized from hexane-ethyl acetate. melting point: 141-142° C.

### Example 240

To a mixture of 3-{3-(1-ethylpropyl)-1-[5-(trifluoromethyl)-2-pyridinyl]-1H-pyrazol-4-yl}-1-propanol (460 mg), methyl (3-ethyl-2-hydroxyphenyl)acetate (270 mg), tributylphosphine (580 mg) and tetrahydrofuran (30 ml) was added 1,1′-azodicarbonyldipiperidine (730 mg) at room temperature and the mixture was stirred overnight at 65° C. The reaction solution was concentrated and isopropyl ether was added. The insoluble material was removed by filtration and the filtrate was concentrated. Then, the residue was subjected to silica gel column chromatography, and methyl [3-ethyl-2-(3-{3-(1-ethylpropyl)-1-[5-(trifluoromethyl)-2-pyridinyl]-1H-pyrazol-4-yl}propoxy)phenyl]acetate (510 mg, yield 73%) was obtained as a colorless oil from a fraction eluted with ethyl acetate-hexane (1:9, volume ratio).

¹H-NMR (CDCl₃)δ: 0.89 (6H, t, J=7.2 Hz), 1.24 (3H, t, J=7.6 Hz), 1.60-1.88 (4H,m), 2.09-2.16 (2H, m), 2.50-2.72 (5H, m), 3.69 (2H, s), 3.69 (3H, s), 3.87 (2H, t, J=6.0 Hz), 7.04 (1H, t, J=7.6 Hz), 7.11 (1H, dd, J=7.6, 2.0 Hz), 7.15 (1H, dd, J=7.6, 2.0 Hz), 7.95 (1H, dd, J=8.8, 2.4 Hz), 8.04 (1H, d, J=8.8 Hz), 8.33 (1H, s), 8.61 (1H, m).

### Example 241

A mixture of methyl [3-ethyl-2-(3-{3-(1-ethylpropyl)-1-[5-(trifluoromethyl)-2-pyridinyl]-1H-pyrazol-4-yl}propoxy)phenyl]acetate (480 mg), 1N aqueous sodium hydroxide solution (7 ml), tetrahydrofuran (5 ml) and methanol (5 ml) was stirred at room temperature 4 hours and 1N hydrochloric acid (7 ml) was added. The mixture was extracted with ethyl acetate. The ethyl acetate layer was washed with saturated aqueous sodium chloride solution, dried (MgSO₄) and concentrated. The obtained colorless crystals were collected by filtration to give [3-ethyl-2-(3-{3-(1-ethylpropyl)-1-[5-(trifluoromethyl)-2-pyridinyl]-1H-pyrazol-4-yl}propoxy)phenyl]acetic acid (350 mg, yield 75%). The crystals were recrystallized from hexane-ethyl acetate. melting point: 130-131° C.

### Example 242

To a mixture of 3-[1-(5-chloro-2-pyridinyl)-3-isopropyl-1H-pyrazol-4-yl]-1-propanol (70 mg), methyl (3-ethyl-2-hydroxyphenyl)acetate (50 mg), tributylphosphine (110 mg) and tetrahydrofuran (10 ml) was added 1,1′-azodicarbonyldipiperidine (140 mg) at room temperature and the mixture was stirred overnight at 65° C. The reaction solution was concentrated and isopropyl ether was added. The insoluble material was removed by filtration and the filtrate was concentrated. Then, the residue was subjected to silica gel column chromatography, and methyl (2-{3-[1-(5-chloro-2-pyridinyl)-3-isopropyl-1H-pyrazol-4-yl]propoxy}-3-ethylphenyl)acetate (80 mg, yield 69%) was obtained as a colorless oil from a fraction eluted with ethyl acetate-hexane (1:9, volume ratio).

¹H-NMR (CDCl₃)δ: 1.24 (3H, t, J=7.6 Hz), 1.34 (6H, d, J=6.8 Hz), 2.05-2.20 (2H, m), 2.56-2.80 (4H, m), 3.04-3.10 (1H,m), 3.68 (2H, s), 3.68 (3H, s), 3.86 (2H, t, J=6.4 Hz), 7.04 (1H, t, J=7.6 Hz), 7.05-7.16 (2H, m), 7.71 (1H, dd, J=8.8, 2.8 Hz), 7.90 (1H, dd, J=8.8, 0.8 Hz), 8.24 (1H, s), 8.29 (1H, m).

### Example 243

A mixture of methyl (2-{3-[1-(5-chloro-2-pyridinyl)-3-isopropyl-1H-pyrazol-4-yl]propoxy}-3-ethylphenyl)acetate (80 mg), 1N aqueous sodium hydroxide solution (2 ml), tetrahydrofuran (2 ml) and methanol (2 ml) was stirred at room temperature for 4 hours. 1N Hydrochloric acid (2 ml) was added and the mixture was extracted with ethyl acetate. The ethyl acetate layer was washed with saturated aqueous sodium chloride solution, dried (MgSO₄) and concentrated. The obtained colorless crystals were collected by filtration to give (2-{3-[1-(5-chloro-2-pyridinyl)-3-isopropyl-1H-pyrazol-4-yl]propoxy}-3-ethylphenyl)acetic acid (50 mg, yield 62%). The crystals were recrystallized from hexane-ethyl acetate. melting point: 142-143° C.

### Example 244

To a mixture of 3-{3-isopropyl-1-[6-(trifluoromethyl)pyridazin-3-yl]-1H-pyrazol-4-yl}-1-propanol (300 mg), methyl (3-ethyl-2-hydroxyphenyl)acetate (190 mg), tributylphosphine (410 mg) and tetrahydrofuran (30 ml) was added 1,1′-azodicarbonyldipiperidine (520 mg) at room temperature and the mixture was stirred overnight at 65° C. The reaction solution was concentrated and isopropyl ether was added. The insoluble material was removed by filtration and the filtrate was concentrated. Then, the residue was subjected to silica gel column chromatography, and methyl [3-ethyl-2-(3-{3-isopropyl-1-[6-(trifluoromethyl)pyridazin-3-yl]-1H-pyrazol-4-yl}propoxy)phenyl]acetate (250 mg, yield 53%) was obtained as a colorless oil from a fraction eluted with ethyl acetate-hexane (1:9, volume ratio).

¹H-NMR (CDCl₃)δ: 1.24 (3H, t, J=7.6 Hz), 1.35 (6H, d, J=6.8 Hz), 2.10-2.19 (2H, m), 2.65-2.72 (2H, m), 2.77 (2H, q, J=7.6 Hz), 3.05-3.11 (1H,m), 3.68 (2H, s), 3.69 (3H, s), 3.89 (2H, t, J=6.0 Hz), 7.05 (1H, t, J=7.6 Hz), 7.11 (1H, dd, J=7.6, 1.6 Hz), 7.15 (1H, dd, J=7.6, 1.6 Hz), 7.84 (1H, d, J-9.2 Hz), 8.30 (1H, d, J=9.2 Hz), 8.55 (1H, s).

### Example 245

A mixture of methyl [3-ethyl-2-(3-{3-isopropyl-1-[6-(trifluoromethyl)pyridazin-3-yl]-1H-pyrazol-4-yl}propoxy)phenyl]acetate (220 mg), 1N aqueous sodium hydroxide solution (7 ml), tetrahydrofuran (5 ml) and methanol (5 ml) was stirred at room temperature for 4 hours. 1N Hydrochloric acid (7 ml) was added, and the mixture was extracted with ethyl acetate. The ethyl acetate layer was washed with saturated aqueous sodium chloride solution, dried (MgSO₄) and concentrated. The obtained colorless crystals were collected by filtration to give [3-ethyl-2-(3-{3-isopropyl-1-[6-(trifluoromethyl)pyridazin-3-yl]-1H-pyrazol-4-yl}propoxy)phenyl]acetic acid (140 mg, yield 67%). The crystals were recrystallized from hexane-ethyl acetate. melting point: 126-127° C.

### Example 246

To a mixture of 3-[1-(5-bromo-2-pyridinyl)-3-isopropyl-1H-pyrazol-4-yl]-1-propanol (250 mg), methyl (3-ethyl-2-hydroxyphenyl)acetate (150 mg), tributylphosphine (330 mg) and tetrahydrofuran (20 ml) was added 1,1′-azodicarbonyldipiperidine (410 mg) at room temperature and the mixture was stirred overnight at 65° C. The reaction solution was concentrated and isopropyl ether was added. The insoluble material was removed by filtration and the filtrate was concentrated. Then, the residue was subjected to silica gel column chromatography, and methyl (2-{3-[1-(5-bromo-2-pyridinyl)-3-isopropyl-1H-pyrazol-4-yl]propoxy}-3-ethylphenyl)acetate (210 mg, yield 54%) was obtained as a colorless oil from a fraction eluted with ethyl acetate-hexane (1:9, volume ratio).

¹H-NMR (CDCl₃)δ: 1.24 (3H, t, J=7.6 Hz), 1.34 (6H, d, J=7.2 Hz), 2.05-2.17 (2H, m), 2.64-2.74 (4H, m), 3.02-3.09 (1H, m), 3.68 (2H, s), 3.68 (3H, s), 3.86 (2H, t, J=6.4 Hz), 7.04 (1H, t, J=7.6 Hz), 7.10 (1H, dd, J=7.6, 1.6 Hz), 7.15 (1H, dd, J=7.6, 1.6 Hz), 7.84-7.85 (2H, m), 8.24 (1H, s), 8.38-8.39 (1H, m).

### Example 247

A mixture of methyl (2-{3-[1-(5-bromo-2-pyridinyl)-3-isopropyl-1H-pyrazol-4-yl]propoxy}-3-ethylphenyl)acetate (190 mg), 1N aqueous sodium hydroxide solution (2 ml), tetrahydrofuran (2 ml) and methanol (2 ml) was stirred at room temperature for 4 hours. 1N Hydrochloric acid (2 ml) was added and the mixture was extracted with ethyl acetate. The ethyl acetate layer was washed with saturated aqueous sodium chloride solution, dried (MgSO₄) and concentrated. The obtained colorless crystals were collected by filtration to give (2-{3-[1-(5-bromo-2-pyridinyl)-3-isopropyl-1H-pyrazol-4-yl]propoxy}-3-ethylphenyl)acetic acid (140 mg, yield 76%). The crystals were recrystallized from hexane-ethyl acetate. melting point: 152° C.

### Example 248

To a mixture of 3-{1-[3-chloro-5-(trifluoromethyl)-2-pyridinyl]-3-isopropyl-1H-pyrazol-4-yl}-1-propanol (250 mg), methyl (3-ethyl-2-hydroxyphenyl)acetate (140 mg), tributylphosphine (310 mg) and tetrahydrofuran (20 ml) was added 1,1′-azodicarbonyldipiperidine (390 mg) at room temperature and the mixture was stirred overnight at 65° C. The reaction solution was concentrated and isopropyl ether was added. The insoluble material was removed by filtration and the filtrate was concentrated. Then, the residue was subjected to silica gel column chromatography, and methyl [2-(3-{1-[3-chloro-5-(trifluoromethyl)-2-pyridinyl]-3-isopropyl-1H-pyrazol-4-yl}propoxy)-3-ethylphenyl]acetate (180 mg, yield 48%) was obtained as a colorless oil from a fraction eluted with ethyl acetate-hexane (1:9, volume ratio).

¹H-NMR (CDCl₃)δ: 1.24 (3H, t, J=7.6 Hz), 1.37 (6H, d, J=7.2 Hz), 2.02-2.18 (2H, m), 2.64-2.77 (4H, m), 3.06-3.12 (1H, m), 3.68 (2H, s), 3.68 (3H, s), 3.88 (2H, t, J=6.4 Hz), 7.05 (1H, t, J=7.6 Hz), 7.10-7.12 (1H, m), 7.15 (1H, dd, J=7.6, 2.0 Hz), 8.08 (2H, m), 8.62 (1H, m).

### Example 249

A mixture of methyl [2-(3-{1-[3-chloro-5-(trifluoromethyl)-2-pyridinyl]-3-isopropyl-1H-pyrazol-4-yl}propoxy)-3-ethylphenyl]acetate (160 mg), 1N aqueous sodium hydroxide solution (2 ml), tetrahydrofuran (2 ml) and methanol (2 ml) was stirred at room temperature for 4 hours. 1N Hydrochloric acid (2 ml) was added and the mixture was extracted with ethyl acetate. The ethyl acetate layer was washed with saturated aqueous sodium chloride solution, dried (MgSO₄) and concentrated. The obtained colorless crystals were collected by filtration to give [2-(3-{1-[3-chloro-5-(trifluoromethyl)-2-pyridinyl]-3-isopropyl-1H-pyrazol-4-yl}propoxy)-3-ethylphenyl]acetic acid (90 mg, yield 58%). The crystals were recrystallized from hexane-ethyl acetate. melting point: 93-95° C.

### Example 250

To a mixture of 3-[1-(3,5-dichloro-2-pyridinyl)-3-isopropyl-1H-pyrazol-4-yl]-1-propanol (250 mg), methyl (3-ethyl-2-hydroxyphenyl)acetate (160 mg), tributylphosphine (340 mg) and tetrahydrofuran (20 ml) was added 1,1′-azodicarbonyldipiperidine (430 mg) at room temperature and the mixture was stirred overnight at 65° C. The reaction solution was concentrated and isopropyl ether was added. The insoluble material was removed by filtration and the filtrate was concentrated. Then, the residue was subjected to silica gel column chromatography, and methyl (2-{3-[1-(3,5-dichloro-2-pyridinyl)-3-isopropyl-1H-pyrazol-4-yl]propoxy}-3-ethylphenyl)acetate (260 mg, yield 67%) was obtained as a colorless oil from a fraction eluted with ethyl acetate-hexane (1:9, volume ratio).

¹H-NMR (CDCl₃)δ: 1.23 (3H, t, J=7.6 Hz), 1.36 (6H, d, J=6.8 Hz), 2.10-2.16 (2H, m), 2.67 (2H, q, J=7.6 Hz), 2.74 (2H, t, J=8.4 Hz), 3.06-3.13 (1H, m), 3.68 (2H, s) 3.68 (3H, s), 3.87 (2H, t, J=6.4 Hz), 7.04 (1H, t, J=7.6 Hz), 7.11 (1H, dd, J=7.6, 1.6 Hz), 7.15 (1H, dd, J=7.6, 1.6 Hz), 7.87 (1H, d, J=2.4 Hz),7.89 (1H, s), 8.35-8.36 (1H, m).

### Example 251

A mixture of methyl (2-{3-[1-(3,5-dichloro-2-pyridinyl)-3-isopropyl-1H-pyrazol-4-yl]propoxy}-3-ethylphenyl)acetate (230 mg), 1N aqueous sodium hydroxide solution (3 ml), tetrahydrofuran (3 ml) and methanol (3 ml) was stirred at room temperature for 4 hours. 1N Hydrochloric acid (3 ml) was added, and the mixture was extracted with ethyl acetate. The ethyl acetate layer was washed with saturated aqueous sodium chloride solution, dried (MgSO₄) and concentrated. The obtained colorless crystals were collected by filtration to give (2-{3-[1-(3,5-dichloro-2-pyridinyl)-3-isopropyl-1H-pyrazol-4-yl]propoxy}-3-ethylphenyl)acetic acid (180 mg, yield 81%). The crystals were recrystallized from hexane-ethyl acetate. melting point: 93-95° C.

### Example 252

To a mixture of 3-[1-(5-chloro-2-pyridinyl)-3-(1-ethylpropyl)-1H-pyrazol-4-yl]-1-propanol (530 mg), methyl (3-ethyl-2-hydroxyphenyl)acetate (340 mg), tributylphosphine (740 mg) and tetrahydrofuran (30 ml) was added 1,1′-azodicarbonyldipiperidine (920 mg) at room temperature and the mixture was stirred overnight at 65° C. The reaction solution was concentrated and isopropyl ether was added. The insoluble material was removed by filtration and the filtrate was concentrated. Then, the residue was subjected to silica gel column chromatography, and methyl (2-{3-[1-(5-chloro-2-pyridinyl)-3-(1-ethylpropyl)-1H-pyrazol-4-yl]propoxy}-3-ethylphenyl)acetate (440 mg, yield 53%) was obtained as a colorless oil from a fraction eluted with ethyl acetate-hexane (1:9, volume ratio).

¹H-NMR (CDCl₃)δ: 0.88 (6H, t, J=7.6 Hz), 1.23 (3H, t, J=7.6 Hz), 1.70-1.79 (4H, m), 2.10-2.13 (2H, m), 2.55-2.71 (5H, m), 3.68 (2H, s), 3.68 (3H, s), 3.86 (2H, t, J=6.4 Hz), 7.04 (1H, t, J=7.6 Hz), 7.12 (1H, dd, J=7.6, 2.0 Hz), 7.15 (1H, dd, J=7.6, 2.0 Hz), 7.70 (1H, dd, J=8.8, 2.4 Hz), 7.89 (1H, dd, J=8.8, 0.4 Hz), 8.24 (1H, s), 8.29-8.30 (1H, m).

### Example 253

A mixture of methyl (2-{3-[1-(5-chloro-2-pyridinyl)-3-(1-ethylpropyl)-1H-pyrazol-4-yl]propoxy}-3-ethylphenyl)acetate (440 mg), 1N aqueous sodium hydroxide solution (3 ml), tetrahydrofuran (3 ml) and methanol (3 ml) was stirred at room temperature for 4 hours. 1N Hydrochloric acid (3 ml) was added, and the mixture was extracted with ethyl acetate. The ethyl acetate layer was washed with saturated aqueous sodium chloride solution, dried (MgSO₄) and concentrated. The obtained colorless crystals were collected by filtration to give (2-{3-[1-(5-chloro-2-pyridinyl)-3-(1-ethylpropyl)-1H-pyrazol-4-yl]propoxy}-3-ethylphenyl)acetic acid (300 mg, yield 70%). The crystals were recrystallized from hexane-ethyl acetate. melting point: 114-115° C.

### Example 254

To a solution of 4-propyl-3-(3-{3-propyl-1-[5-(trifluoromethyl)-2-pyridyl]-1H-pyrazol-4-yl}propoxy)-1H-pyrazole (400 mg) in N,N-dimethylformamide (6 ml) was added sodium hydride (60%, in oil, 46 mg) at 0° C. and the mixture was stirred at room temperature for 15 minutes. Bromomethyl acetate (0.10 ml) was added at 0° C. and the mixture was stirred at room temperature for 1 hour. The reaction mixture was poured into water, and extracted with ethyl acetate. The ethyl acetate layer was washed with saturated aqueous sodium chloride solution, dried (MgSO₄) and concentrated. The residue was subjected to silica gel column chromatography, and white crystals were obtained from a fraction eluted with ethyl acetate-hexane (1:2, volume ratio). A mixture of the obtained crystal, 1N aqueous sodium hydroxide solution (1.5 ml), tetrahydrofuran (4 ml) and methanol (4 ml) was stirred at room temperature 5 hours. 1N Hydrochloric acid (1.5 ml) was added, and the mixture was extracted with ethyl acetate. The ethyl acetate layer was washed with saturated aqueous sodium chloride solution, dried (MgSO₄) and concentrated. The obtained colorless crystals were collected by filtration to give [4-propyl-3-(3-{3-propyl-1-[5-(trifluoromethyl)-2-pyridyl]-1H-pyrazol-4-yl}propoxy)-1H-pyrazol-1-yl]acetic acid (310 mg, yield 69%). The crystals were recrystallized from ethyl acetate-hexane. melting point: 138-139° C.

### Example 255

To a solution of 3-(3-{3-ethoxy-1-[5-(trifluoromethyl)-2-pyridyl]-1H-pyrazol-4-yl}propoxy)-4-propyl-1H-pyrazole (400 mg) in N,N-dimethylformamide (6 ml) was added sodium hydride (60%, in oil, 46 mg) at 0° C. and the mixture was stirred at room temperature for 15 minutes. Bromomethyl acetate (0.10 ml) was added at 0° C. and the mixture was stirred at room temperature for 1 hour. The reaction mixture was poured into water, and extracted with ethyl acetate. The ethyl acetate layer was washed with saturated aqueous sodium chloride solution, dried (MgSO₄) and concentrated. The residue was subjected to silica gel column chromatography, and white crystals were obtained from a fraction eluted with ethyl acetate-hexane (1:2, volume ratio). A mixture of the obtained crystal, 1N aqueous sodium hydroxide solution (1.5 ml), tetrahydrofuran (4 ml) and methanol (4 ml) was stirred at room temperature 5 hours. 1N Hydrochloric acid (1.5 ml) was added, and the mixture was extracted with ethyl acetate. The ethyl acetate layer was washed with saturated aqueous sodium chloride solution, dried (MgSO₄) and concentrated. The obtained colorless crystals were collected by filtration to give [3-(3-{3-ethoxy-1-[5-(trifluoromethyl)-2-pyridyl]-1H-pyrazol-4-yl}propoxy)-4-propyl-1H-pyrazol-1-yl]acetic acid (330 mg, yield 72%). The crystals were recrystallized from ethyl acetate-hexane. melting point: 134-135° C.

### Example 256

To a mixture of 3-{3-propyl-1-[5-(trifluoromethyl)-2-pyridyl]-1H-pyrazol-4-yl}-1-propanol (400 mg), ethyl 3-(1-cyclohexyl-3-hydroxy-1H-pyrazol-5-yl)propanoate (370 mg), tributylphosphine (520 mg) and tetrahydrofuran (30 ml) was added 1,1′-azodicarbonyldipiperidine (640 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (2 ml), tetrahydrofuran (4 ml) and methanol (4 ml) was stirred at room temperature for 5 hours. 1N Hydrochloric acid (2 ml) was added and the mixture was extracted with ethyl acetate. The ethyl acetate layer was washed with saturated aqueous sodium chloride solution, dried (MgSO₄) and concentrated. The obtained colorless crystals were collected by filtration to give 3-[1-cyclohexyl-3-(3-{3-propyl-1-[5-(trifluoromethyl)-2-pyridyl]-1H-pyrazol-4-yl}propoxy)-1H-pyrazol-5-yl]propanoic acid (240 mg, yield 35%). The crystals were recrystallized from ethyl acetate-hexane. melting point: 164-165° C.

### Example 257

To a mixture of 3-{3-ethoxy-1-[5-(trifluoromethyl)-2-pyridyl]-1H-pyrazol-4-yl}-1-propanol (400 mg), ethyl 3-(1-cyclohexyl-3-hydroxy-1H-pyrazol-5-yl)propanoate (370 mg), tributylphosphine (520 mg) and tetrahydrofuran (30 ml) was added 1,1′-azodicarbonyldipiperidine (640 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (2 ml), tetrahydrofuran (4 ml) and methanol (4 ml) was stirred at room temperature for 5 hours. 1N Hydrochloric acid (2 ml) was added and the mixture was extracted with ethyl acetate. The ethyl acetate layer was washed with saturated aqueous sodium chloride solution, dried (MgSO₄) and concentrated. The obtained colorless crystals were collected by filtration to give 3-[1-cyclohexyl-3-(3-{3-ethoxy-1-[5-(trifluoromethyl)-2-pyridyl]-1H-pyrazol-4-yl}propoxy)-1H-pyrazol-5-yl]propanoic acid (270 mg, yield 40%). The crystals were recrystallized from ethyl acetate-hexane. melting point: 89-90° C.

### Example 258

To a mixture of 3-{3-propyl-1-[5-(trifluoromethyl)-2-pyridyl]-1H-pyrazol-4-yl}-1-propanol (230 mg), ethyl (2-fluoro-3-hydroxyphenyl)acetate (140 mg), tributylphosphine (280 mg) and tetrahydrofuran (30 ml) was added 1,1′-azodicarbonyldipiperidine (350 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (1.5 ml), tetrahydrofuran (4 ml) and methanol (4 ml) was stirred at room temperature for 5 hours. 1N Hydrochloric acid (1.5 ml) was added, and the mixture was extracted with ethyl acetate. The ethyl acetate layer was washed with saturated aqueous sodium chloride solution, dried (MgSO₄) and concentrated. The obtained colorless crystals were collected by filtration to give [2-fluoro-3-(3-{3-propyl-1-[5-(trifluoromethyl)-2-pyridyl]-1H-pyrazol-4-yl}propoxy)phenyl]acetic acid (220 mg, yield 68%). The crystals were recrystallized from ethyl acetate-hexane. melting point: 109-110° C.

### Example 259

To a mixture of 3-{3-propyl-1-[5-(trifluoromethyl)-2-pyridyl]-1H-pyrazol-4-yl}-1-propanol (500 mg), methyl (2-hydroxy-3-methoxyphenyl)acetate (310 mg), tributylphosphine (640 mg) and tetrahydrofuran (35 ml) was added 1,1′-azodicarbonyldipiperidine (800 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (2.5 ml), tetrahydrofuran (4 ml) and methanol (4 ml) was stirred at room temperature for 5 hours. 1N Hydrochloric acid (2.5 ml) was added, and the mixture was extracted with ethyl acetate. The ethyl acetate layer was washed with saturated aqueous sodium chloride solution, dried (MgSO₄) and concentrated. The obtained colorless crystals were collected by filtration to give [3-methoxy-2-(3-{3-propyl-1-[5-(trifluoromethyl)-2-pyridyl]-1H-pyrazol-4-yl}propoxy)phenyl]acetic acid (590 mg, yield 78%). The crystals were recrystallized from ethyl acetate-hexane. melting point: 122-123° C.

### Example 260

To a mixture of 3-{3-propyl-1-[5-(trifluoromethyl)-2-pyridyl]-1H-pyrazol-4-yl}-1-propanol (620 mg), methyl (3-ethoxy-2-hydroxyphenyl)acetate (420 mg), tributylphosphine (800 mg) and tetrahydrofuran (35 ml) was added 1,1′-azodicarbonyldipiperidine (1.00 g) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (3 ml), tetrahydrofuran (4 ml) and methanol (4 ml) was stirred at room temperature for 5 hours. 1N Hydrochloric acid (3 ml) was added, and the mixture was extracted with ethyl acetate. The ethyl acetate layer was washed with saturated aqueous sodium chloride solution, dried (MgSO₄) and concentrated. The obtained colorless crystals were collected by filtration to give [3-ethoxy-2-(3-{3-propyl-1-[5-(trifluoromethyl)-2-pyridyl]-1H-pyrazol-4-yl}propoxy)phenyl]acetic acid (880 mg, yield 90%). The crystals were recrystallized from ethyl acetate-hexane. melting point: 133-134° C.

### Example 261

To a mixture of 3-{3-cyclohexyl-1-[5-(trifluoromethyl)-2-pyridyl]-1H-pyrazol-4-yl}-1-propanol (400 mg), ethyl 3-(1-cyclohexyl-3-hydroxy-1H-pyrazol-5-yl)propanoate (300 mg), tributylphosphine (460 mg) and tetrahydrofuran (30 ml) was added 1,1′-azodicarbonyldipiperidine (570 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (2 ml), tetrahydrofuran (4 ml) and methanol (4 ml) was stirred at room temperature for 5 hours. 1N Hydrochloric acid (2 ml) was added and the mixture was extracted with ethyl acetate. The ethyl acetate layer was washed with saturated aqueous sodium chloride solution, dried (MgSO₄) and concentrated. The obtained colorless crystals were collected by filtration to give 3-[1-cyclohexyl-3-(3-{3-cyclohexyl-1-[5-(trifluoromethyl)-2-pyridyl]-1H-pyrazol-4-yl}propoxy)-1H-pyrazol-5-yl]propanoic acid (300 mg, yield 46%). The crystals were recrystallized from ethyl acetate-hexane. melting point: 190-191° C.

### Example 262

To a mixture of 3-{3-isopropyl-1-[5-(trifluoromethyl)-2-pyridyl]-1H-pyrazol-4-yl}-1-propanol (400 mg), ethyl 3-(1-cyclohexyl-3-hydroxy-1H-pyrazol-5-yl)propanoate (340 mg), tributylphosphine (520 mg) and tetrahydrofuran (30 ml) was added 1,1′-azodicarbonyldipiperidine (650 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (2 ml), tetrahydrofuran (4 ml) and methanol (4 ml) was stirred at room temperature for 5 hours. 1N Hydrochloric acid (2 ml) was added and the mixture was extracted with ethyl acetate. The ethyl acetate layer was washed with saturated aqueous sodium chloride solution, dried (MgSO₄) and concentrated. The obtained colorless crystals were collected by filtration to give 3-[1-cyclohexyl-3-(3-{3-isopropyl-1-[5-(trifluoromethyl)-2-pyridyl]-1H-pyrazol-4-yl}propoxy)-1H-pyrazol-5-yl]propanoic acid (230 mg, yield 34%). The crystals were recrystallized from ethyl acetate-hexane. melting point: 166-167° C.

### Example 263

To a mixture of 3-{3-isopropyl-1-[5-(trifluoromethyl)-2-pyridyl]-1H-pyrazol-4-yl}-1-propanol (350 mg), ethyl 3-hydroxy-1-isopropyl-1H-pyrazol-5-yl]propanoate (250 mg), tributylphosphine (440 mg) and tetrahydrofuran (50 ml) was added 1,1′-azodicarbonyldipiperidine (560 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (1 ml), tetrahydrofuran (4 ml) and methanol (4 ml) was stirred at room temperature for 5 hours. 1N Hydrochloric acid (1 ml) was added, and the mixture was extracted with ethyl acetate. The ethyl acetate layer was washed with saturated aqueous sodium chloride solution, dried (MgSO₄) and concentrated. The obtained colorless crystals were collected by filtration to give 3-[1-isopropyl-3-(3-{3-isopropyl-1-[5-(trifluoromethyl)-2-pyridyl]-1H-pyrazol-4-yl}propoxy)-1H-pyrazol-5-yl]propanoic acid (200 mg, yield 36%). The crystals were recrystallized from ethyl acetate-hexane. melting point: 130-131° C.

### Example 264

A mixture of methyl (2-{3-[1-(5-amino-2-pyridyl)-3-isopropyl-1H-pyrazol-4-yl]propoxy}-3-methoxyphenyl)acetate (390 mg), 1N aqueous sodium hydroxide solution (1.5 ml), tetrahydrofuran (4 ml) and methanol (4 ml) was stirred at room temperature for 5 hours, and concentrated. To a mixture of the obtained residue and water (10 ml) was added a solution of calcium chloride (0.20 g) in water (1 ml) at room temperature and the mixture was stirred overnight. The resulting white precipitates were collected by filtration to give calcium (2-{3-[1-(5-amino-2-pyridyl)-3-isopropyl-1H-pyrazol-4-yl]propoxy}-3-methoxyphenyl)acetate (300 mg, yield 81%) as amorphous.

¹H-NMR (DMSO-d₆)δ: 1.23 (6H, d, J=6.6 Hz), 1.85-1.98 (2H, m), 2.60 (2H, t, J=7.4 Hz), 2.96 (1H, septet, J=6.9 Hz), 3.37 (2H, s), 3.73 (3H, s), 3.91 (2H, t, J=6.2 Hz), 5.29 (2H, br s), 6.75-6.90 (3H, m), 7.08 (1H, dd, J=8.6, 2.9 Hz), 7.51 (1H, d, J=8.4 Hz), 7.72 (1H, d, J=2.4 Hz), 8.10 (1H, s).

### Example 265

To a mixture of methyl (2-{3-[1-(5-amino-2-pyridyl)-3-isopropyl-1H-pyrazol-4-yl]propoxy}-3-methoxyphenyl)acetate (390 mg) and N,N-dimethylformamide (6 ml) was added acetic anhydride (0.10 ml) at room temperature and the mixture was stirred overnight. Water (20 ml) was added to the reaction mixture and the mixture was extracted with ethyl acetate. The ethyl acetate layer was washed with aqueous sodium hydrogen carbonate, dried (MgSO₄), and concentrated. A mixture of the obtained residue, 1N aqueous sodium hydroxide solution (1.5 ml), tetrahydrofuran (4 ml) and methanol (4 ml) was stirred at room temperature for 5 hours. 1N Hydrochloric acid (1.5 ml) was added, and the mixture was extracted with ethyl acetate. The ethyl acetate layer was washed with saturated aqueous sodium chloride solution, dried (MgSO₄) and concentrated. The obtained colorless crystals were collected by filtration to give (2-{3-[1-(5-acetylamino-2-pyridyl)-3-isopropyl-1H-pyrazol-4-yl]propoxy}-3-methoxyphenyl)acetic acid (320 mg, yield 78%). The crystals were recrystallized from ethyl acetate-hexane. melting point: 158-159° C.

### Example 266

To a mixture of methyl (2-{3-[1-(5-amino-2-pyridyl)-3-isopropyl-1H-pyrazol-4-yl]propoxy}-3-methoxyphenyl)acetate (400 mg) and N,N-dimethylformamide (6 ml) was added propionyl chloride (0.12 ml) at 0° C. and the mixture was stirred at room temperature overnight. Water (20 ml) was added to the reaction mixture, and the mixture was extracted with ethyl acetate. The ethyl acetate layer was washed with aqueous sodium hydrogen carbonate, dried (MgSO₄) and concentrated. The residue was subjected to silica gel column chromatography, and a colorless oil was obtained from a fraction eluted with ethyl acetate-hexane (1:1, volume ratio). A mixture of the obtained oily substance, 1N aqueous sodium hydroxide solution (2 ml), tetrahydrofuran (4 ml) and methanol (4 ml) was stirred at room temperature for 5 hours. 1N Hydrochloric acid (2 ml) was added and the mixture was extracted with ethyl acetate. The ethyl acetate layer was washed with saturated aqueous sodium chloride solution, dried (MgSO₄) and concentrated. The obtained colorless crystals were collected by filtration to give (3-methoxy-2-{3-[3-isopropyl-1-(5-propanoylamino-2-pyridyl)-1H-pyrazol-4-yl]propoxy}phenyl)acetic acid (340 mg, yield 78%). The crystals were recrystallized from ethyl acetate-hexane. melting point: 147-148° C.

### Example 267

To a mixture of methyl (2-{3-[1-(5-amino-2-pyridyl)-3-isopropyl-1H-pyrazol-4-yl]propoxy}-3-methoxyphenyl)acetate (400 mg) and N,N-dimethylformamide (6 ml) was added butyryl chloride (0.14 ml) at 0° C. and the mixture was stirred at room temperature overnight. Water (20 ml) was added to the reaction mixture, and the mixture was extracted with ethyl acetate. The ethyl acetate layer was washed with aqueous sodium hydrogen carbonate, dried (MgSO₄) and concentrated. The residue was subjected to silica gel column chromatography, and a colorless oil was obtained from a fraction eluted with ethyl acetate-hexane (1:1, volume ratio). A mixture of the obtained oily substance, 1N aqueous sodium hydroxide solution (2 ml), tetrahydrofuran (4 ml) and methanol (4 ml) was stirred at room temperature for 5 hours. 1N Hydrochloric acid (2 ml) was added and the mixture was extracted with ethyl acetate. The ethyl acetate layer was washed with saturated aqueous sodium chloride solution, dried (MgSO₄) and concentrated. The obtained colorless crystals were collected by filtration to give (2-{3-[1-(5-butyrylamino-2-pyridyl)-3-isopropyl-1H-pyrazol-4-yl]propoxy}-3-methoxyphenyl)acetic acid (350 mg, yield 83%). The crystals were recrystallized from ethyl acetate-hexane. melting point: 119-120° C.

### Example 268

To a mixture of methyl (2-{3-[1-(5-amino-2-pyridyl)-3-isopropyl-1H-pyrazol-4-yl]propoxy}-3-methoxyphenyl)acetate (400 mg) and N,N-dimethylformamide (6 ml) was added isobutyryl chloride (0.15 ml) at 0° C. and the mixture was stirred at room temperature overnight. Water (20 ml) was added to the reaction mixture, and the mixture was extracted with ethyl acetate. The ethyl acetate layer was washed with aqueous sodium hydrogen carbonate, dried (MgSO₄) and concentrated. The residue was subjected to silica gel column chromatography, and a colorless oil was obtained from a fraction eluted with ethyl acetate-hexane (1:1, volume ratio). A mixture of the obtained oily substance, 1N aqueous sodium hydroxide solution (2 ml), tetrahydrofuran (4 ml) and methanol (4 ml) was stirred at room temperature for 5 hours. 1N Hydrochloric acid (2 ml) was added and the mixture was extracted with ethyl acetate. The ethyl acetate layer was washed with saturated aqueous sodium chloride solution, dried (MgSO₄) and concentrated. The obtained colorless crystals were collected by filtration to give [3-methoxy-2-(3-{3-isopropyl-1-[5-(2-methylpropanoylamino)-2-pyridyl]-1H-pyrazol-4-yl}propoxy)phenyl]acetic acid (370 mg, yield 82%). The crystals were recrystallized from ethyl acetate-hexane. melting point: 122-123° C.

### Example 269

To a mixture of methyl (2-{3-[1-(5-amino-2-pyridyl)-3-isopropyl-1H-pyrazol-4-yl]propoxy}-3-methoxyphenyl)acetate (400 mg), pyridine (0.10 ml) and acetonitrile (6 ml) was added methanesulfonyl chloride (0.10 ml) at 0° C., and the mixture was stirred at room temperature for 1 hour. Water (15 ml) was added to the reaction mixture, and the mixture was extracted with ethyl acetate. The ethyl acetate layer was washed with saturated aqueous sodium chloride solution, dried (MgSO₄) and concentrated. The residue was subjected to silica gel column chromatography, and a colorless oil was obtained from a fraction eluted with ethyl acetate-hexane (1:1, volume ratio). A mixture of the obtained oily substance, 1N aqueous sodium hydroxide solution (2 ml), tetrahydrofuran (4 ml) and methanol (4 ml) was stirred at room temperature for 5 hours. 1N Hydrochloric acid (2 ml) was added and the mixture was extracted with ethyl acetate. The ethyl acetate layer was washed with saturated aqueous sodium chloride solution, dried (MgSO₄) and concentrated. The obtained colorless crystals were collected by filtration to give (3-methoxy-2-{3-[3-isopropyl-1-(5-methylsulfonylamino-2-pyridyl)-1H-pyrazol-4-yl]propoxy}phenyl)acetic acid (270 mg, yield 58%). The crystals were recrystallized from ethanol. melting point: 176-177° C.

### Example 270

A mixture of methyl (3-methoxy-2-{3-[3-isopropyl-1-(5-nitro-2-pyridyl)-1H-pyrazol-4-yl]propoxy}phenyl)acetate (400 mg), 1N aqueous sodium hydroxide solution (1.5 ml), tetrahydrofuran (4 ml) and methanol (4 ml) was stirred at room temperature for 5 hours. 1N Hydrochloric acid (1.5 ml) was added, and the mixture was extracted with ethyl acetate. The ethyl acetate layer was washed with saturated aqueous sodium chloride solution, dried (MgSO₄) and concentrated. The obtained colorless crystals were collected by filtration to give (3-methoxy-2-{3-[3-isopropyl-1-(5-nitro-2-pyridyl)-1H-pyrazol-4-yl]propoxy}phenyl)acetic acid (240 mg, yield 62%). The crystals were recrystallized from ethanol. melting point: 161-162° C.

### Example 271

To a mixture of 6-[4-(3-hydroxypropyl)-3-isopropyl-1H-pyrazol-1-yl]pyridine-3-carbonitrile (520 mg), methyl (2-hydroxy-3-methoxyphenyl)acetate (380 mg), tributylphosphine (780 mg) and tetrahydrofuran (30 ml) was added 1,1′-azodicarbonyldipiperidine (970 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (2 ml), tetrahydrofuran (4 ml) and methanol (4 ml) was stirred at room temperature for 5 hours. 1N Hydrochloric acid (2 ml) was added and the mixture was extracted with ethyl acetate. The ethyl acetate layer was washed with saturated aqueous sodium chloride solution, dried (MgSO₄) and concentrated. The residue was subjected to silica gel column chromatography, and (2-{3-[1-(5-cyano-2-pyridyl)-3-isopropyl-1H-pyrazol-4-yl]propoxy}-3-methoxyphenyl)acetic acid (460 mg, yield 67%) was obtained as colorless crystals from a fraction eluted with ethyl acetate-hexane (3:1, volume ratio). The crystals were recrystallized from ethyl acetate-hexane. melting point: 144-145° C.

### Example 272

To a mixture of 3-[3-isopropyl-1-(5-methyl-2-pyridyl)-1H-pyrazol-4-yl]-1-propanol (400 mg), methyl (2-hydroxy-3-methoxyphenyl)acetate (300 mg), tributylphosphine (620 mg) and tetrahydrofuran (50 ml) was added 1,1′-azodicarbonyldipiperidine (780 mg) at room temperature and the mixture was stirred overnight. The reaction solution was concentrated. The residue was subjected to silica gel column chromatography, and a colorless oil was obtained from a fraction eluted with ethyl acetate-hexane (1:3, volume ratio). A mixture of the obtained oily substance, 1N aqueous sodium hydroxide solution (2.5 ml), tetrahydrofuran (4 ml) and methanol (4 ml) was stirred at room temperature for 5 hours. 1N Hydrochloric acid (2.5 ml) was added, and the mixture was extracted with ethyl acetate. The ethyl acetate layer was washed with saturated aqueous sodium chloride solution, dried (MgSO₄) and concentrated. The obtained colorless crystals were collected by filtration to give (3-methoxy-2-{3-[3-isopropyl-1-(5-methyl-2-pyridyl)-1H-pyrazol-4-yl]propoxy}phenyl)acetic acid (470 mg, yield 72%). The crystals were recrystallized from ethyl acetate-hexane. melting point: 132-133° C.

### Example 273

To a mixture of 3-[1-(5-fluoro-2-pyridyl)-3-isopropyl-1H-pyrazol-4-yl]-1-propanol (500 mg), methyl (2-hydroxy-3-methoxyphenyl)acetate (410 mg), tributylphosphine (770 mg) and tetrahydrofuran (50 ml) was added 1,1′-azodicarbonyldipiperidine (960 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (2.5 ml), tetrahydrofuran (4 ml) and methanol (4 ml) was stirred at room temperature for 5 hours. 1N Hydrochloric acid (2.5 ml) was added, and the mixture was extracted with ethyl acetate. The ethyl acetate layer was washed with saturated aqueous sodium chloride solution, dried (MgSO₄) and concentrated. The obtained colorless crystals were collected by filtration to give (2-{3-[1-(5-fluoro-2-pyridyl)-3-isopropyl-1H-pyrazol-4-yl]propoxy}-3-methoxyphenyl)acetic acid (180 mg, yield 22%). The crystals were recrystallized from ethanol-hexane. melting point: 125-126° C.

### Example 274

To a mixture of 3-{3-isopropyl-1-[6-(trifluoromethyl)pyridazin-3-yl]-1H-pyrazol-4-yl}-1-propanol (400 mg), methyl (2-hydroxy-3-methoxyphenyl)acetate (280 mg), tributylphosphine (520 mg) and tetrahydrofuran (30 ml) was added 1,1′-azodicarbonyldipiperidine (640 mg) at room temperature and the mixture was stirred overnight. The reaction solution was concentrated. The residue was subjected to silica gel column chromatography, and a colorless oil was obtained from a fraction eluted with ethyl acetate-hexane (1:3, volume ratio). A mixture of the obtained oily substance, 1N aqueous sodium hydroxide solution (2 ml), tetrahydrofuran (4 ml) and methanol (4 ml) was stirred at room temperature for 5 hours. 1N Hydrochloric acid (2 ml) was added and the mixture was extracted with ethyl acetate. The ethyl acetate layer was washed with saturated aqueous sodium chloride solution, dried (MgSO₄) and concentrated. The obtained colorless crystals were collected by filtration to give [2-(3-{3-isopropyl-1-[6-(trifluoromethyl)pyridazin-3-yl]-1H-pyrazol-4-yl}propoxy)-3-methoxyphenyl]acetic acid (490 mg, yield 80%). The crystals were recrystallized from ethyl acetate-hexane. melting point: 92-93° C.

### Example 275

To a mixture of 3-{3-isopropyl-1-[5-(trifluoromethyl)pyrimidin-2-yl]-1H-pyrazol-4-yl}-1-propanol (400 mg), benzyl (2-hydroxy-3-methoxyphenyl)acetate (450 mg), tributylphosphine (520 mg) and tetrahydrofuran (30 ml) was added 1,1′-azodicarbonyldipiperidine (640 mg) at room temperature and the mixture was stirred overnight. The reaction solution was concentrated. The residue was subjected to silica gel column chromatography, and a colorless oil was obtained from a fraction eluted with ethyl acetate-hexane (1:3, volume ratio). A mixture of the obtained oily substance, 5% palladium-carbon (0.1 g) and tetrahydrofuran (8 ml) was stirred overnight at room temperature under a hydrogen atmosphere. Palladium-carbon was removed by filtration and the filtrate was concentrated. The residue was subjected to silica gel column chromatography, and [3-methoxy-2-(3-{3-isopropyl-1-[5-(trifluoromethyl)pyrimidin-2-yl]-1H-pyrazol-4-yl}propoxy)phenyl]acetic acid (390 mg, yield 65%) was obtained as colorless crystals from a fraction eluted with ethyl acetate-hexane (2:3, volume ratio). The crystals were recrystallized from ethyl acetate-hexane. melting point: 131-132° C.

### Example 276

To a mixture of 3-[1-(5-ethylpyrimidin-2-yl)-3-isopropyl-1H-pyrazol-4-yl]-1-propanol (400 mg), methyl (2-hydroxy-3-methoxyphenyl)acetate (310 mg), tributylphosphine (590 mg) and tetrahydrofuran (30 ml) was added 1,1′-azodicarbonyldipiperidine (740 mg) at room temperature and the mixture was stirred overnight. The reaction solution was concentrated. The residue was subjected to silica gel column chromatography, and a colorless oil was obtained from a fraction eluted with ethyl acetate-hexane (2:3, volume ratio). A mixture of the obtained oily substance, 1N aqueous sodium hydroxide solution (2 ml), tetrahydrofuran (4 ml) and methanol (4 ml) was stirred at room temperature for 5 hours. 1N Hydrochloric acid (2 ml) was added and the mixture was extracted with ethyl acetate. The ethyl acetate layer was washed with saturated aqueous sodium chloride solution, dried (MgSO₄) and concentrated. The obtained colorless crystals were collected by filtration to give (2-{3-[1-(5-ethylpyrimidin-2-yl)-3-isopropyl-1H-pyrazol-4-yl]propoxy}-3-methoxyphenyl)acetic acid (430 mg, yield 68%). The crystals were recrystallized from ethyl acetate-hexane. melting point: 103-104° C.

### Example 277

To a mixture of 3-[1-(6-methoxypyridazin-3-yl)-3-isopropyl-1H-pyrazol-4-yl]-1-propanol (300 mg), methyl (2-hydroxy-3-methoxyphenyl)acetate (240 mg), tributylphosphine (440 mg) and tetrahydrofuran (30 ml) was added 1,1′-azodicarbonyldipiperidine (550 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (1.5 ml), tetrahydrofuran (4 ml) and methanol (4 ml) was stirred at room temperature for 5 hours. 1N Hydrochloric acid (1.5 ml) was added, and the mixture was extracted with ethyl acetate. The ethyl acetate layer was washed with saturated aqueous sodium chloride solution, dried (MgSO₄) and concentrated. The obtained colorless crystals were collected by filtration to give (3-methoxy-2-{3-[1-(6-methoxypyridazin-3-yl)-3-isopropyl-1H-pyrazol-4-yl]propoxy}phenyl)acetic acid (400 mg, yield 84%). The crystals were recrystallized from ethyl acetate-hexane. melting point: 124-125° C.

### Example 278

To a mixture of 3-{1-[5-(trifluoromethyl)-2-pyridyl]-3-isopropyl-1H-pyrazol-4-yl}-1-propanol (400 mg), ethyl (4-hydroxy-3-methoxyphenyl)acetate (280 mg), tributylphosphine (520 mg) and tetrahydrofuran (50 ml) was added 1,1′-azodicarbonyldipiperidine (650 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (1.5 ml), tetrahydrofuran (4 ml) and methanol (4 ml) was stirred at room temperature for 5 hours. 1N Hydrochloric acid (1.5 ml) was added, and the mixture was extracted with ethyl acetate. The ethyl acetate layer was washed with saturated aqueous sodium chloride solution, dried (MgSO₄) and concentrated. The obtained colorless crystals were collected by filtration to give [3-methoxy-4-(3-{1-[5-(trifluoromethyl)-2-pyridyl]-3-isopropyl-1H-pyrazol-4-yl}propoxy)phenyl]acetic acid (250 mg, yield 41%). The crystals were recrystallized from ethyl acetate-hexane. melting point: 134-135° C.

### Example 279

To a mixture of 3-{1-[5-(trifluoromethyl)-2-pyridyl]-3-isopropyl-1H-pyrazol-4-yl}-1-propanol (400 mg), ethyl 3-(4-hydroxy-3-methoxyphenyl)propanoate (300 mg), tributylphosphine (520 mg) and tetrahydrofuran (50 ml) was added 1,1′-azodicarbonyldipiperidine (650 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (1.5 ml), tetrahydrofuran (4 ml) and methanol (4 ml) was stirred at room temperature for 5 hours. 1N Hydrochloric acid (1.5 ml) was added, and the mixture was extracted with ethyl acetate. The ethyl acetate layer was washed with saturated aqueous sodium chloride solution, dried (MgSO₄) and concentrated. The obtained colorless crystals were collected by filtration to give 3-[3-methoxy-4-(3-{1-[5-(trifluoromethyl)-2-pyridyl]-3-isopropyl-1H-pyrazol-4-yl}propoxy)phenyl]propanoic acid (220 mg, yield 35%). The crystals were recrystallized from ethyl acetate-hexane. melting point: 118-119° C.

### Example 280

To a mixture of 6-[4-(3-hydroxypropyl)-3-isopropyl-1H-pyrazol-1-yl]pyridazine-3-carbonitrile (400 mg), methyl (2-hydroxy-3-methoxyphenyl)acetate (320 mg), tributylphosphine (600 mg) and tetrahydrofuran (30 ml) was added 1,1′-azodicarbonyldipiperidine (740 mg) at room temperature and the mixture was stirred overnight. The reaction solution was concentrated. The residue was subjected to silica gel column chromatography, and a colorless oil was obtained from a fraction eluted with ethyl acetate-hexane (1:3, volume ratio). A mixture of the obtained oily substance, 1N aqueous sodium hydroxide solution (2 ml), tetrahydrofuran (4 ml) and methanol (4 ml) was stirred at room temperature for 2 hours. 1N Hydrochloric acid (2 ml) was added and the mixture was extracted with ethyl acetate. The ethyl acetate layer was washed with saturated aqueous sodium chloride solution, dried (MgSO₄) and concentrated. The residue was subjected to silica gel column chromatography, and 6-(4-{3-[2-methoxy-6-(methoxycarbonylmethyl)phenoxy]propyl}-3-isopropyl-1H-pyrazol-1-yl)pyridazine-3-carboxylic acid (220 mg, yield 32%) was obtained as colorless crystals from a fraction eluted with ethyl acetate-hexane (1:1, volume ratio). The crystals were recrystallized from ethyl acetate-hexane. melting point: 146-147° C.

### Example 281

To a mixture of 3-[3-isopropyl-1-(5-chloro-2-pyridinyl)-1H-pyrazol-4-yl]-1-propanol (400 mg), methyl (2-hydroxyphenyl)acetate (261 mg), tributylphosphine (713 μL) and tetrahydrofuran (100 ml) was added 1,1′-azodicarbonyldipiperidine (722 mg) at room temperature and the mixture was stirred for 2.5 days. The reaction solution was concentrated. The residue was subjected to silica gel column chromatography, and methyl (2-{3-[1-(5-chloro-2-pyridinyl)-3-isopropyl-1H-pyrazol-4-yl]propoxy}phenyl)acetate (470 mg, yield 77%) was obtained as a pale-yellow oily substance from a fraction eluted with ethyl acetate-hexane (1:12, volume ratio).

¹H-NMR (CDCl₃) δ: 1.32 (6H, d, J=6.9 Hz), 2.02-2.18 (2H, m), 2.62-2.71 (2H, m), 2.95-3.10 (1H, m), 3.65 (2H, s), 3.67 (3H, s), 4.00-4.06 (2H, m), 6.80-6.94 (2H, m), 7.15-7.27 (2H, m), 7.69 (1H, dd, J=2.7, 9.0 Hz), 7.88 (1H, d, J=9.0 Hz), 8.19 (1H, s), 8.27 (1H, d, J=2.7 Hz).

### Example 282

To a mixture of 3-[3-isopropyl-1-(5-chloro-2-pyridinyl)-1H-pyrazol-4-yl]-1-propanol (400 mg), methyl (2-hydroxy-3-methoxyphenyl)acetate (308 mg), tributylphosphine (713 μL) and tetrahydrofuran (100 ml) was added 1,1′-azodicarbonyldipiperidine (722 mg) at room temperature and the mixture was stirred overnight. The reaction solution was concentrated. The residue was subjected to silica gel column chromatography, and methyl (2-{3-[1-(5-chloro-2-pyridinyl)-3-isopropyl-1H-pyrazol-4-yl]propoxy}-3-methoxyphenyl)acetate (550 mg, yield 84%) was obtained as a colorless oil from a fraction eluted with ethyl acetate-hexane (1:12, volume ratio).

¹H-NMR (CDCl₃) δ: 1.33 (6H, d, J=6.9 Hz), 2.01-2.12 (2H, m), 2.64-2.72 (2H, m), 2.97-3.12 (1H, m), 3.66 (3H, s), 3.68 (2H, s), 3.83 (3H, s), 4.02-4.09 (2H, m), 6.78-6.86 (2H, m), 6.96-7.03 (1H, m), 7.68 (1H, dd, J=2.7, 9.0 Hz), 7.88 (1H, d, J=9.0 Hz), 8.23 (1H, s), 8.27 (1H, d, J=2.7 Hz).

### Example 283

To a mixture of 3-{(3-methyl-1-[5-(trifluoromethyl)-2-pyridinyl]-1H-pyrazol-4-yl}-1-propanol (367 mg), methyl (3-hydroxy-1-methyl-1H-pyrazol-5-yl)acetate (200 mg), tributylphosphine (588 μL) and tetrahydrofuran (25 ml) was added 1,1′-azodicarbonyldipiperidine (596 mg) at room temperature and the mixture was stirred overnight. The reaction solution was concentrated. The residue was subjected to silica gel column chromatography, and a yellow oily substance was obtained from a fraction eluted with ethyl acetate-hexane (1:1, volume ratio). A mixture of the obtained oily substance, 1N aqueous sodium hydroxide solution (25 ml), tetrahydrofuran (25 ml) and ethanol (25 ml) was stirred overnight at room temperature. 1N Hydrochloric acid (25 ml) was added, and the mixture was extracted with ethyl acetate. The ethyl acetate layer was washed with saturated aqueous sodium chloride solution, dried (MgSO₄) and concentrated. The obtained pale-red solid was recrystallized from ethyl acetate to give [1-methyl-3-(3-{3-methyl-1-[5-(trifluoromethyl)-2-pyridinyl]-1H-pyrazol-4-yl}propoxy)-1H-pyrazol-5-yl]acetic acid (222 mg, yield 44%) as colorless crystals. melting point: 143-144° C.

### Example 284

To a mixture of 2-{3-ethoxy-1-[5-(trifluoromethyl)-2-pyridinyl]-1H-pyrazol-4-yl}-1-ethanol (356 mg), methyl (3-hydroxy-1-methyl-1H-pyrazol-5-yl)acetate (200 mg), tributylphosphine (588 μL) and tetrahydrofuran (25 ml) was added 1,1′-azodicarbonyldipiperidine (596 mg) at room temperature and the mixture was stirred overnight. The reaction solution was concentrated. The residue was subjected to silica gel column chromatography, and a yellow oily substance was obtained from a fraction eluted with ethyl acetate-hexane (1:3, volume ratio). A mixture of the obtained oily substance, 1N aqueous sodium hydroxide solution (25 ml), tetrahydrofuran (25 ml) and ethanol (25 ml) was stirred overnight at room temperature. 1N Hydrochloric acid (25 ml) was added, and the mixture was extracted with ethyl acetate. The ethyl acetate layer was washed with saturated aqueous sodium chloride solution, dried (MgSO₄) and concentrated. The residue was subjected to silica gel column chromatography, and a white solid was obtained from a fraction eluted with ethyl acetate-hexane (7:3, volume ratio). The obtained solid was recrystallized from ethyl acetate-hexane to give [3-(2-{3-ethoxy-1-[5-(trifluoromethyl)-2-pyridinyl]-1H-pyrazol-4-yl}ethoxy)-1-methyl-1H-pyrazol-5-yl]acetic acid (104 mg, yield 20%) as colorless crystals. melting point: 156-158° C.

### Example 285

To a mixture of 3-{3-ethoxy-1-[4-(trifluoromethyl)-2-pyridinyl]-1H-pyrazol-4-yl}-1-propanol (500 mg), methyl 3-(2-ethoxy-4-hydroxyphenyl)propanoate (392 mg), tributylphosphine (792 μL) and tetrahydrofuran (32 ml) was added 1,1′-azodicarbonyldipiperidine (802 mg) at room temperature and the mixture was stirred overnight. The reaction solution was concentrated. The residue was subjected to silica gel column chromatography, and a white solid was obtained from a fraction eluted with ethyl acetate-hexane (1:5, volume ratio). The obtained solid was recrystallized from ethyl acetate-hexane to give methyl 3-[2-ethoxy-4-(3-{3-ethoxy-1-[4-(trifluoromethyl)-2-pyridinyl]-1H-pyrazol-4-yl}propoxy)phenyl]propanoate (174 mg, yield 34%) as colorless crystals. melting point: 79-81° C.

### Example 286

To a mixture of 3-{3-ethoxy-1-[4-(trifluoromethyl)-2-pyridinyl]-1H-pyrazol-4-yl}-1-propanol (500 mg), methyl (3-hydroxyphenyl)acetate (529 mg), tributylphosphine (792 μL) and tetrahydrofuran (32 ml) was added 1,1′-azodicarbonyldipiperidine (802 mg) at room temperature and the mixture was stirred overnight. The reaction solution was concentrated. The residue was subjected to silica gel column chromatography, and a white solid was obtained from a fraction eluted with ethyl acetate-hexane (1:5, volume ratio). A mixture of the obtained solid, 1N aqueous sodium hydroxide solution (25 ml), tetrahydrofuran (25 ml) and ethanol (25 ml) was stirred overnight at room temperature. 1N Hydrochloric acid (25 ml) was added, and the mixture was extracted with ethyl acetate. The ethyl acetate layer was washed with saturated aqueous sodium chloride solution, dried (MgSO₄) and concentrated. The obtained white solid was recrystallized from ethyl acetate-hexane to give [3-(3-{3-ethoxy-1-[4-(trifluoromethyl)-2-pyridinyl]-1H-pyrazol-4-yl}propoxy)phenyl]acetic acid (524 mg, yield 73%) as colorless crystals. melting point: 128-130° C.

### Example 287

To a mixture of 3-{3-ethoxy-1-[4-(trifluoromethyl)-2-pyridinyl]-1H-pyrazol-4-yl}-1-propanol (500 mg), ethyl 3-(3-ethoxy-4-hydroxyphenyl)propanoate (393 mg), tributylphosphine (792 μL) and tetrahydrofuran (50 ml) was added 1,1′-azodicarbonyldipiperidine (802 mg) at room temperature and the mixture was stirred overnight. The reaction solution was concentrated. The residue was subjected to silica gel column chromatography, and a white solid was obtained from a fraction eluted with ethyl acetate-hexane (1:5, volume ratio). A mixture of the obtained solid, 1N aqueous sodium hydroxide solution (25 ml), tetrahydrofuran (25 ml) and ethanol (25 ml) was stirred overnight at room temperature. 1N Hydrochloric acid (25 ml) was added, and the mixture was extracted with ethyl acetate. The ethyl acetate layer was washed with saturated aqueous sodium chloride solution, dried (MgSO₄) and concentrated. The obtained white solid was recrystallized from ethyl acetate-hexane to give 3-[4-(3-{3-ethoxy-1-[4-(trifluoromethyl)-2-pyridinyl]-1H-pyrazol-4-yl}propoxy)-3-methoxyphenyl]propanoic acid (403 mg, yield 51%) as colorless crystals. melting point: 111-112° C.

### Example 288

To a mixture of 3-{3-ethoxy-1-[4-(trifluoromethyl)-2-pyridinyl]-1H-pyrazol-4-yl}-1-propanol (500 mg), methyl (2-hydroxyphenyl)acetate (291 mg), tributylphosphine (792 μL) and tetrahydrofuran (50 ml) was added 1,1′-azodicarbonyldipiperidine (802 mg) at room temperature and the mixture was stirred overnight. The reaction solution was concentrated. The residue was subjected to silica gel column chromatography, and a white solid was obtained from a fraction eluted with ethyl acetate-hexane (1:5, volume ratio). A mixture of the obtained solid, 1N aqueous sodium hydroxide solution (25 ml), tetrahydrofuran (25 ml) and ethanol (25 ml) was stirred overnight at room temperature. 1N Hydrochloric acid (25 ml) was added, and the mixture was extracted with ethyl acetate. The ethyl acetate layer was washed with saturated aqueous sodium chloride solution, dried (MgSO₄) and concentrated. The obtained white solid was recrystallized from ethyl acetate-hexane to give [2-(3-{3-ethoxy-1-[4-(trifluoromethyl)-2-pyridinyl]-1H-pyrazol-4-yl}propoxy)phenyl]acetic acid (479 mg, yield 67%) as colorless crystals. melting point: 121-122° C.

### Example 289

To a mixture of 3-{3-ethoxy-1-[4-(trifluoromethyl)-2-pyridinyl]-1H-pyrazol-4-yl}-1-propanol (500 mg), ethyl 2-(3-hydroxyphenoxy)-2-methylpropaneacetate (393 mg), tributylphosphine (792 μL) and tetrahydrofuran (100 ml) was added 1,1′-azodicarbonyldipiperidine (802 mg) at room temperature and the mixture was stirred overnight. The reaction solution was concentrated. The residue was subjected to silica gel column chromatography, and a colorless oil was obtained from a fraction eluted with ethyl acetate-hexane (1:7, volume ratio). A mixture of the obtained oily substance, 1N aqueous sodium hydroxide solution (25 ml), tetrahydrofuran (25 ml) and ethanol (25 ml) was stirred overnight at room temperature. 1N Hydrochloric acid (25 ml) was added, and the mixture was extracted with ethyl acetate. The ethyl acetate layer was washed with saturated aqueous sodium chloride solution, dried (MgSO₄) and concentrated. The residue was subjected to silica gel column chromatography, and a colorless oil was obtained from a fraction eluted with ethyl acetate-hexane (1:1, volume ratio). The obtained oily substance was recrystallized from ethyl acetate-hexane to give 2-[3-(3-{3-ethoxy-1-[4-(trifluoromethyl)-2-pyridinyl]-1H-pyrazol-4-yl}propoxy)phenoxy]-2-methylpropanoic acid (140 mg, yield 18%) as colorless crystals. melting point: 98-99° C.

### Example 290

To a mixture of ethyl 2-{3-[3-(3-ethoxy-1H-pyrazol-4-yl)propoxy]phenoxy}-2-methylpropanoate (400 mg), 2-chloro-3-(trifluoromethyl)pyridine (240 mg) and N,N-dimethylformamide (20 ml) was added sodium hydride (60%, in oil, 52.8 mg) at 0° C. and the mixture was stirred at room temperature for 3 hours. Thereafter, to the reaction solution was added ethyl iodide (106 μL), and the mixture was stirred for 2.5 hours. To the reaction solution was added saturated aqueous ammonium chloride solution, and the mixture was extracted with ethyl acetate. The ethyl acetate layer was washed with saturated aqueous sodium chloride solution, dried (MgSO₄) and concentrated. The residue was subjected to silica gel column chromatography, and a colorless oil was obtained from a fraction eluted with ethyl acetate-hexane (1:5, volume ratio). A mixture of the obtained oily substance, 1N aqueous sodium hydroxide solution (25 ml), tetrahydrofuran (25 ml) and ethanol (25 ml) was stirred overnight at room temperature. 1N Hydrochloric acid (25 ml) was added, and the mixture was extracted with ethyl acetate. The ethyl acetate layer was washed with saturated aqueous sodium chloride solution, dried (MgSO₄) and concentrated. A mixture of the obtained colorless oil, 1N aqueous sodium hydroxide solution (325 μL), tetrahydrofuran (25 ml) and ethanol (25 ml) was stirred at room temperature for 1 hour, and concentrated. To a mixture of the obtained residue and water (50 ml) was added calcium chloride (36.0 mg) dissolved in a small amount of water and the mixture was stirred overnight at room temperature. The resulting white precipitates were collected by filtration to give calcium 2-[3-(3-{3-ethoxy-1-[3-(trifluoromethyl)-2-pyridinyl]-1H-pyrazol-4-yl}propoxy)phenoxy]-2-methylpropanoate (166 mg, yield 31%) as amorphous.

¹H-NMR (DMSO-d₆) δ: 1.33 (3H, t, J=7.2 Hz), 1.41 (6H, s), 1.92-2.04 (2H, m), 2.45-2.55 (2H, m), 3.88-3.96 (2H, m), 4.24 (2H, q, J=7.2 Hz), 6.36-6.45 (3H, m), 6.96-7.04 (1H, m), 7.44-7.51 (1H, m), 8.19 (1H, s) 8.29-8.35 (1H, m), 8.63-8.68 (1H, m).

### Example 291

A mixture of ethyl 2-{3-[3-(3-ethoxy-1H-pyrazol-4-yl)propoxy]phenoxy}-2-methylpropanoate (400 mg), 3-(trifluoromethyl)phenylboric acid (418 mg), copper(II) acetate (300 mg), pyridine (160 μL) and N,N-dimethylformamide (10 ml) was stirred overnight at room temperature. To the reaction solution was added saturated aqueous ammonium chloride solution, and the mixture was extracted with ethyl acetate. The ethyl acetate layer was washed with saturated aqueous sodium chloride solution, dried (MgSO₄) and concentrated. The residue was subjected to silica gel column chromatography, and a colorless oil was obtained from a fraction eluted with ethyl acetate-hexane (1:5, volume ratio). A mixture of the obtained oily substance, 1N aqueous sodium hydroxide solution (25 ml), tetrahydrofuran (25 ml) and ethanol (25 ml) was stirred overnight at room temperature. 1N Hydrochloric acid (25 ml) was added, and the mixture was extracted with ethyl acetate. The ethyl acetate layer was washed with saturated aqueous sodium chloride solution, dried (MgSO₄) and concentrated. The residue was subjected to silica gel column chromatography, and a colorless oil was obtained from a fraction eluted with ethyl acetate-hexane (1:1, volume ratio). A mixture of the obtained oily substance, 1N aqueous sodium hydroxide solution (610 μL), tetrahydrofuran (25 ml) and ethanol (25 ml) was stirred at room temperature for 1 hour, and concentrated. To a mixture of the obtained residue and water (50 ml) was added calcium chloride (67.6 mg) dissolved in a small amount of water and the mixture was stirred overnight at room temperature. The resulting white precipitates were collected by filtration to give calcium 2-[3-(3-{3-ethoxy-1-[3-(trifluoromethyl)phenyl]-1H-pyrazol-4-yl}propoxy)phenoxy]-2-methylpropanoate (343 mg, yield 63%) as amorphous.

¹H-NMR (DMSO-d₆) δ: 1.34 (3H, t, J=7.0 Hz), 1.41 (6H, s), 1.90-2.07 (2H, m), 2.42-2.54 (2H, m), 3.88-4.00 (2H, m), 4.29 (2H, q, J=7.0 Hz), 6.36-6.46 (3H, m), 6.96-7.07 (1H, m), 7.45-7.53 (1H, m), 7.59-7.70 (1H, m), 7.95-8.04 (2H, m), 8.41 (1H, s).

### Example 292

A mixture of ethyl 2-{3-[3-(3-ethoxy-1H-pyrazol-4-yl)propoxy]phenoxy}-2-methylpropanoate (400 mg), 2-(trifluoromethyl)phenylboric acid (418 mg), copper(II) acetate (300 mg), pyridine (160 μL) and N,N-dimethylformamide (10 ml) was stirred overnight at room temperature. To the reaction solution was added saturated aqueous ammonium chloride solution, and the mixture was extracted with ethyl acetate. The ethyl acetate layer was washed with saturated aqueous sodium chloride solution, dried (MgSO₄) and concentrated. The residue was subjected to silica gel column chromatography, and a colorless oil was obtained from a fraction eluted with ethyl acetate-hexane (1:5, volume ratio). A mixture of the obtained oily substance, 1N aqueous sodium hydroxide solution (25 ml), tetrahydrofuran (25 ml) and ethanol (25 ml) was stirred overnight at room temperature. 1N Hydrochloric acid (25 ml) was added, and the mixture was extracted with ethyl acetate. The ethyl acetate layer was washed with saturated aqueous sodium chloride solution, dried (MgSO₄) and concentrated. The residue was subjected to silica gel column chromatography, and a colorless oil was obtained from a fraction eluted with ethyl acetate-hexane (1:1, volume ratio). A mixture of the obtained oily substance, 1N aqueous sodium hydroxide solution (265 μL), tetrahydrofuran (25 ml) and ethanol (25 ml) was stirred at room temperature for 1 hour, and concentrated. To a mixture of the obtained residue and water (50 ml) was added calcium chloride (29.3 mg) dissolved in a small amount of water and the mixture was stirred overnight at room temperature. The resulting white precipitates were collected by filtration to give calcium 2-[3-(3-{3-ethoxy-1-[2-(trifluoromethyl)phenyl]-1H-pyrazol-4-yl}propoxy)phenoxy]-2-methylpropanoate (109 mg, yield 20%) as amorphous.

¹H-NMR (DMSO-d₆) δ: 1.30 (3H, t, J=7.0 Hz), 1.41 (6H, s), 1.86-2.04 (2H, m), 2.42-2.54 (2H, m), 3.86-3.98 (2H, m), 4.19 (2H, q, j=7.0 Hz), 6.35-6.47 (3H, m), 6.96-7.06 (1H, m), 7.53-7.65 (2H, m), 7.68-7.90 (3H, m).

### Example 293

A mixture of ethyl 2-{3-[3-(3-ethoxy-1H-pyrazol-4-yl)propoxy]phenoxy}-2-methylpropanoate (400 mg), 4-ethylphenylboric acid (318 mg), copper(II) acetate (289 mg), pyridine (154 μL) and N,N-dimethylformamide (10 ml) was stirred overnight at room temperature. To the reaction solution was added saturated aqueous ammonium chloride solution, and the mixture was extracted with ethyl acetate. The ethyl acetate layer was washed with saturated aqueous sodium chloride solution, dried (MgSO₄) and concentrated. The residue was subjected to silica gel column chromatography, and a colorless oil was obtained from a fraction eluted with ethyl acetate-hexane (1:9, volume ratio). A mixture of the obtained oily substance, 1N aqueous sodium hydroxide solution (25 ml), tetrahydrofuran (25 ml) and ethanol (25 ml) was stirred overnight at room temperature. 1N Hydrochloric acid (25 ml) was added, and the mixture was extracted with ethyl acetate. The ethyl acetate layer was washed with saturated aqueous sodium chloride solution, dried (MgSO₄) and concentrated. The residue was subjected to silica gel column chromatography, and a colorless oil was obtained from a fraction eluted with ethyl acetate-hexane (1:1, volume ratio). A mixture of the obtained oily substance, 1N aqueous sodium hydroxide solution (600 μL), tetrahydrofuran (25 ml) and ethanol (25 ml) was stirred at room temperature for 1 hour, and concentrated. To a mixture of the obtained residue and water (50 ml) was added calcium chloride (66.2 mg) dissolved in a small amount of water and the mixture was stirred overnight at room temperature. The resulting white precipitates were collected by filtration to give calcium 2-(3-{3-[3-ethoxy-1 (4-ethylphenyl)-1H-pyrazol-4-yl]propoxy}phenoxy)-2-methylpropanoate (274 mg, yield 55%) as amorphous.

¹H-NMR (DMSO-d₆) δ: 1.18 (3H, t, J=7.8 Hz), 1.33 (3H, t, J=7.0 Hz), 1.41 (6H, s), 1.88-2.06 (2H, m), 2.40-2.55 (2H, m), 2.59 (2H, q, J=7.8 Hz), 3.86-3.98 (2H, m), 4.25 (2H, q, J=7.0 Hz), 6.35-6.46 (3H, m), 6.95-7.07 (1H, m), 7.23 (2H, d, J=8.8 Hz), 7.57 (2H, d, J=8.8 Hz), 8.13 (1H, s).

### Example 294

A mixture of ethyl 2-{3-[3-(3-ethoxy-1H-pyrazol-4-yl)propoxy]phenoxy}-2-methylpropanoate (400 mg), 4-methylphenylboric acid (288 mg), copper(II) acetate (289 mg), pyridine (154 μL) and N,N-dimethylformamide (10 ml) was stirred overnight at room temperature. To the reaction solution was added saturated aqueous ammonium chloride solution, and the mixture was extracted with ethyl acetate. The ethyl acetate layer was washed with saturated aqueous sodium chloride solution, dried (MgSO₄) and concentrated. The residue was subjected to silica gel column chromatography, and a colorless oil was obtained from a fraction eluted with ethyl acetate-hexane (1:8, volume ratio). A mixture of the obtained oily substance, 1N aqueous sodium hydroxide solution (25 ml), tetrahydrofuran (25 ml) and ethanol (25 ml) was stirred overnight at room temperature. 1N Hydrochloric acid (25 ml) was added, and the mixture was extracted with ethyl acetate. The ethyl acetate layer was washed with saturated aqueous sodium chloride solution, dried (MgSO₄) and concentrated. The residue was subjected to silica gel column chromatography, and a colorless oil was obtained from a fraction eluted with ethyl acetate-hexane (1:1, volume ratio). A mixture of the obtained oily substance, 1N aqueous sodium hydroxide solution (775 μL), tetrahydrofuran (25 ml) and ethanol (25 ml) was stirred at room temperature for 1 hour, and concentrated. To a mixture of the obtained residue and water (50 ml) was added calcium chloride (86.0 mg) dissolved in a small amount of water and the mixture was stirred overnight at room temperature. The resulting white precipitates were collected by filtration to give calcium 2-(3-{3-[3-ethoxy-1-(4-methylphenyl)-1H-pyrazol-4-yl]propoxy}phenoxy)-2-methylpropanoate (353 mg, yield 73%) as amorphous.

¹H-NMR (DMSO-d₆) δ: 1.32 (3H, t, J=7.0 Hz), 1.41 (6H, s), 1.88-2.04 (2H, m), 2.29 (3H, s), 2.40-2.53 (2H, m), 3.86-3.98 (2H, m), 4.25 (2H, q, J=7.0 Hz), 6.35-6.47 (3H, m), 6.95-7.08 (1H, m), 7.21 (2H, d, J=8.4 Hz), 7.55 (2H, d, J=8.4 Hz), 8.13 (1H, s).

### Example 295

To a mixture of 4-{3-propyl-1-[5-(trifluoromethyl)-2-pyridinyl]-1H-pyrazol-4-yl}-1-butanol (500 mg), methyl (2-hydroxy-3-methoxyphenyl)acetate (330 mg), tributylphosphine (762 μL) and tetrahydrofuran (120 ml) was added 1,1′-azodicarbonyldipiperidine (772 mg) at room temperature and the mixture was stirred overnight. The reaction solution was concentrated. The residue was subjected to silica gel column chromatography, and a colorless oil was obtained from a fraction eluted with ethyl acetate-hexane (1:7, volume ratio). A mixture of the obtained oily substance, 1N aqueous sodium hydroxide solution (25 ml), tetrahydrofuran (25 ml) and ethanol (25 ml) was stirred overnight at room temperature. 1N Hydrochloric acid (25 ml) was added, and the mixture was extracted with ethyl acetate. The ethyl acetate layer was washed with saturated aqueous sodium chloride solution, dried (MgSO₄) and concentrated. The obtained white solid was recrystallized from ethyl acetate-hexane to give [3-methoxy-2-(4-{3-propyl-1-[5-(trifluoromethyl)-2-pyridinyl]-1H-pyrazol-4-yl}butoxy)phenyl]acetic acid (451 mg, yield 60%) as colorless crystals. melting point: 111-112° C.

### Example 296

To a mixture of 3-{3-propyl-1-[4-(trifluoromethyl)phenyl]-1H-pyrazol-4-yl}-1-propanol (400 mg), methyl (2-hydroxyphenyl)acetate (234 mg), tributylphosphine (638 μL) and tetrahydrofuran (100 ml) was added 1,1′-azodicarbonyldipiperidine (646 mg) at room temperature and the mixture was stirred for 2 days. The reaction solution was concentrated. The residue was subjected to silica gel column chromatography, and a colorless oil was obtained from a fraction eluted with ethyl acetate-hexane (1:7, volume ratio). A mixture of the obtained oily substance, 1N aqueous sodium hydroxide solution (25 ml), tetrahydrofuran (25 ml) and ethanol (25 ml) was stirred at room temperature for 2.5 days. 1N Hydrochloric acid (25 ml) was added, and the mixture was extracted with ethyl acetate. The ethyl acetate layer was washed with saturated aqueous sodium chloride solution, dried (MgSO₄) and concentrated. The residue was subjected to silica gel column chromatography, and [2-(3-{3-propyl-1-[4-(trifluoromethyl)phenyl]-1H-pyrazol-4-yl}propoxy)phenyl]acetic acid (340 mg, yield 59%) was obtained as a colorless oil from a fraction eluted with ethyl acetate-hexane (1:2, volume ratio).

¹H-NMR (CDCl₃) δ: 0.99 (3H, t, J=7.4 Hz), 1.60-1.82 (2H, m), 1.91-2.08 (2H, m), 2.54-2.68 (4H, m), 3.65 (2H, s), 3.90-4.00 (2H, m), 6.74-6.92 (2H, m), 7.08-7.26 (2H, m), 7.54-7.73 (5H, m).

### Example 297

To a mixture of 3-{3-propyl-1-[4-(trifluoromethyl)phenyl]-1H-pyrazol-4-yl}-1-propanol (420 mg), methyl (2-hydroxy-3-methoxyphenyl)acetate (290 mg), tributylphosphine (668 μL) and tetrahydrofuran (100 ml) was added 1,1′-azodicarbonyldipiperidine (676 mg) at room temperature and the mixture was stirred overnight for 2.5 days. The reaction solution was concentrated. The residue was subjected to silica gel column chromatography, and a colorless oil was obtained from a fraction eluted with ethyl acetate-hexane (1:7, volume ratio). A mixture of the obtained oily substance, 1N aqueous sodium hydroxide solution (25 ml), tetrahydrofuran (25 ml) and ethanol (25 ml) was stirred overnight at room temperature. 1N Hydrochloric acid (25 ml) was added, and the mixture was extracted with ethyl acetate. The ethyl acetate layer was washed with saturated aqueous sodium chloride solution, dried (MgSO₄) and concentrated. The residue was subjected to silica gel column chromatography, and a white solid was obtained from a fraction eluted with ethyl acetate-hexane (2:3, volume ratio). The obtained solid was recrystallized from diisopropyl ether-hexane to give [3-methoxy-2-(3-{3-propyl-1-[4-(trifluoromethyl)phenyl]-1H-pyrazol-4-yl}propoxy)phenyl]acetic acid (472 mg, yield 74%) as colorless crystals. melting point: 95-96° C.

### Example 298

A mixture of methyl 3-[2-ethoxy-4-(3-{3-ethoxy-1-[4-(trifluoromethyl)-2-pyridinyl]-1H-pyrazol-4-yl}propoxy)phenyl]propanoate (86.3 mg), 1N aqueous sodium hydroxide solution (20 ml), tetrahydrofuran (20 ml) and ethanol (20 ml) was stirred at room temperature for 8 hours. 1N Hydrochloric acid (20 ml) was added, and the mixture was extracted with ethyl acetate. The ethyl acetate layer was washed with saturated aqueous sodium chloride solution, dried (MgSO₄) and concentrated. The obtained white solid was recrystallized from ethyl acetate-hexane to give 3-[2-ethoxy-4-(3-{3-ethoxy-1-[4-(trifluoromethyl)-2-pyridinyl]-1H-pyrazol-4-yl}propoxy)phenyl]propanoic acid (67.3 mg, yield 80%) as colorless crystals. melting point: 119-120° C.

### Example 299

To a mixture of 3-{3-isopropyl-1-[5-(trifluoromethyl)-2-pyridinyl]-1H-pyrazol-4-yl}-1-propanol (400 mg), methyl (3-hydroxy-1-methyl-1H-pyrazol-4-yl)acetate (240 mg), tributylphosphine (638 μL) and tetrahydrofuran (100 ml) was added 1,1′-azodicarbonyldipiperidine (646 mg) at room temperature and the mixture was stirred overnight. The reaction solution was concentrated. The residue was subjected to silica gel column chromatography, and a white solid was obtained from a fraction eluted with ethyl acetate-hexane (1:3, volume ratio). A mixture of the obtained solid, 1N aqueous sodium hydroxide solution (20 ml), tetrahydrofuran (20 ml) and ethanol (20 ml) was stirred overnight at room temperature. 1N Hydrochloric acid (20 ml) was added, and the mixture was extracted with ethyl acetate. The ethyl acetate layer was washed with saturated aqueous sodium chloride solution, dried (MgSO₄) and concentrated. The obtained white solid was recrystallized from ethyl acetate-hexane to give [3-(3-{3-isopropyl-1-[5-(trifluoromethyl)-2-pyridinyl]-1H-pyrazol-4-yl}propoxy)-1-methyl-1H-pyrazol-4-yl]acetic acid (441 mg, yield 76%) as colorless crystals. melting point: 122-123° C.

### Example 300

To a mixture of 3-{3-isopropyl-1-[5-(trifluoromethyl)-2-pyridinyl]-1H-pyrazol-4-yl}-1-propanol (400 mg), methyl (3-ethoxy-2-hydroxyphenyl)acetate (424 mg), tributylphosphine (638 μL) and tetrahydrofuran (100 ml) was added 1,1′-azodicarbonyldipiperidine (646 mg) at room temperature and the mixture was stirred overnight. The reaction solution was concentrated. The residue was subjected to silica gel column chromatography, and a colorless oil was obtained from a fraction eluted with ethyl acetate-hexane (1:7, volume ratio). A mixture of the obtained oily substance, 1N aqueous sodium hydroxide solution (25 ml), tetrahydrofuran (25 ml) and ethanol (25 ml) was stirred at room temperature for 5 hours. 1N Hydrochloric acid (25 ml) was added, and the mixture was extracted with ethyl acetate. The ethyl acetate layer was washed with saturated aqueous sodium chloride solution, dried (MgSO₄) and concentrated. The obtained white solid was recrystallized from ethyl acetate-hexane to give [3-ethoxy-2-(3-{3-isopropyl-1-[5-(trifluoromethyl)-2-pyridinyl]-1H-pyrazol-4-yl}propoxy)phenyl]acetic acid (455 mg, yield 72%) as colorless crystals. melting point: 141-142° C.

### Example 301

To a mixture of 3-{3-ethoxy-1-[5-(trifluoromethyl)-2-pyridinyl]-1H-pyrazol-4-yl}-1-propanol (400 mg), methyl (3-ethoxy-2-hydroxyphenyl)acetate (420 mg), tributylphosphine (633 μL) and tetrahydrofuran (100 ml) was added 1,1′-azodicarbonyldipiperidine (641 mg) at room temperature and the mixture was stirred overnight. The reaction solution was concentrated. The residue was subjected to silica gel column chromatography, and a white solid was obtained from a fraction eluted with ethyl acetate-hexane (1:7, volume ratio). A mixture of the obtained solid, 1N aqueous sodium hydroxide solution (25 ml), tetrahydrofuran (25 ml) and ethanol (25 ml) was stirred at room temperature for 5 hours. 1N Hydrochloric acid (25 ml) was added, and the mixture was extracted with ethyl acetate. The ethyl acetate layer was washed with saturated aqueous sodium chloride solution, dried (MgSO₄) and concentrated. The obtained white solid was recrystallized from ethyl acetate-hexane to give [3-ethoxy-2-(3-{3-ethoxy-1-[5-(trifluoromethyl)-2-pyridinyl]-1H-pyrazol-4-yl}propoxy)phenyl]acetic acid (415 mg, yield 66%) as colorless crystals. melting point: 114-115° C.

### Example 302

To a mixture of 3-{3-cyclohexyl-1-[5-(trifluoromethyl)-2-pyridinyl]-1H-pyrazol-4-yl}-1-propanol (400 mg), methyl (2-hydroxyphenyl)acetate (206 mg), tributylphosphine (563 mL) and tetrahydrofuran (100 ml) was added 1,1′-azodicarbonyldipiperidine (570 mg) at room temperature and the mixture was stirred overnight. The reaction solution was concentrated. The residue was subjected to silica gel column chromatography, and a white solid was obtained from a fraction eluted with ethyl acetate-hexane (1:7, volume ratio). A mixture of the obtained solid, 1N aqueous sodium hydroxide solution (20 ml), tetrahydrofuran (20 ml) and ethanol (20 ml) was stirred at room temperature for 5 hours. 1N Hydrochloric acid (20 ml) was added, and the mixture was extracted with ethyl acetate. The ethyl acetate layer was washed with saturated aqueous sodium chloride solution, dried (MgSO₄) and concentrated. The obtained white solid was recrystallized from ethyl acetate-hexane to give [2-(3-{3-cyclohexyl-1-[5-(trifluoromethyl)-2-pyridinyl]-1H-pyrazol-4-yl}propoxy)phenyl]acetic acid (345 mg, yield 63%) as colorless crystals. melting point: 127-128° C.

### Example 303

To a mixture of 3-{3-cyclohexyl-1-[5-(trifluoromethyl)-2-pyridinyl]-1H-pyrazol-4-yl}-1-propanol (400 mg), methyl (3-hydroxy-1-methyl-1H-pyrazol-4-yl)acetate (211 mg), tributylphosphine (563 μL) and tetrahydrofuran (100 ml) was added 1,1′-azodicarbonyldipiperidine (570 mg) at room temperature and the mixture was stirred for 3 days. The reaction solution was concentrated. The residue was subjected to silica gel column chromatography, and a white solid was obtained from a fraction eluted with ethyl acetate-hexane (1:3, volume ratio). A mixture of the obtained solid, 1N aqueous sodium hydroxide solution (20 ml), tetrahydrofuran (20 ml) and ethanol (20 ml) was stirred at room temperature for 5 hours. 1N Hydrochloric acid (20 ml) was added, and the mixture was extracted with ethyl acetate. The ethyl acetate layer was washed with saturated aqueous sodium chloride solution, dried (MgSO₄) and concentrated. The obtained white solid was recrystallized from ethyl acetate-hexane to give [3-(3-{3-cyclohexyl-1-[5-(trifluoromethyl)-2-pyridinyl]-1H-pyrazol-4-yl}propoxy)-1-methyl-1H-pyrazol-4-yl]acetic acid (457 mg, yield 82%) as colorless crystals. melting point: 157-158° C.

### Example 304

To a mixture of 3-{3-cyclohexyl-1-[5-(trifluoromethyl)-2-pyridinyl]-1H-pyrazol-4-yl}-1-propanol (400 mg), ethyl (3-hydroxy-4-methoxyphenyl)acetate (261 mg), tributylphosphine (563 μL) and tetrahydrofuran (100 ml) was added 1,1′-azodicarbonyldipiperidine (570 mg) at room temperature and the mixture was stirred overnight. The reaction solution was concentrated. The residue was subjected to silica gel column chromatography, and a white solid was obtained from a fraction eluted with ethyl acetate-hexane (1:7, volume ratio). A mixture of the obtained solid, 1N aqueous sodium hydroxide solution (20 ml), tetrahydrofuran (20 ml) and ethanol (20 ml) was stirred at room temperature for 5 hours. 1N Hydrochloric acid (20 ml) was added, and the mixture was extracted with ethyl acetate. The ethyl acetate layer was washed with saturated aqueous sodium chloride solution, dried (MgSO₄) and concentrated. The obtained white solid was recrystallized from ethyl acetate-hexane to give [3-(3-{3-cyclohexyl-1-[5-(trifluoromethyl)-2-pyridinyl]-1H-pyrazol-4-yl}propoxy)-4-methoxyphenyl]acetic acid (268 mg, yield 46%) as colorless crystals. melting point: 117-118° C.

### Example 305

To a mixture of 3-{3-propyl-1-[5-(trifluoromethyl)-2-pyridinyl]-1H-pyrazol-4-yl}-1-propanol (170 mg), methyl (1-ethyl-3-hydroxy-1H-pyrazol-4-yl)acetate (100 mg), tributylphosphine (272 μL) and tetrahydrofuran (50 ml) was added 1,1′-azodicarbonyldipiperidine (275 mg) at room temperature and the mixture was stirred overnight. The reaction solution was concentrated. The residue was subjected to silica gel column chromatography, and a white solid was obtained from a fraction eluted with ethyl acetate-hexane (1:5, volume ratio). A mixture of the obtained solid, 1N aqueous sodium hydroxide solution (15 ml), tetrahydrofuran (15 ml) and ethanol (15 ml) was stirred at room temperature for 5 hours. 1N Hydrochloric acid (15 ml) was added, and the mixture was extracted with ethyl acetate. The ethyl acetate layer was washed with saturated aqueous sodium chloride solution, dried (MgSO₄) and concentrated. The obtained white solid was recrystallized from ethyl acetate-hexane to give [1-ethyl-3-(3-{3-propyl-1-[5-(trifluoromethyl)-2-pyridinyl]-1H-pyrazol-4-yl}propoxy)-1H-pyrazol-4-yl]acetic acid (117 mg, yield 46%) as colorless crystals. melting point: 105-106° C.

### Example 306

To a mixture of 3-{3-cyclohexyl-1-[5-(trifluoromethyl)-2-pyridinyl]-1H-pyrazol-4-yl}-1-propanol (500 mg), methyl (3-ethoxy-2-hydroxyphenyl)acetate (466 mg), tributylphosphine (703 μL) and tetrahydrofuran (100 ml) was added 1,1′-azodicarbonyldipiperidine (712 mg) at room temperature and the mixture was stirred overnight. The reaction solution was concentrated. The residue was subjected to silica gel column chromatography, and a colorless oil was obtained from a fraction eluted with ethyl acetate-hexane (1:12, volume ratio). A mixture of the obtained oily substance, 1N aqueous sodium hydroxide solution (25 ml), tetrahydrofuran (25 ml) and ethanol (25 ml) was stirred at room temperature for 5 hours. 1N Hydrochloric acid (25 ml) was added, and the mixture was extracted with ethyl acetate. The ethyl acetate layer was washed with saturated aqueous sodium chloride solution, dried (MgSO₄) and concentrated. The obtained white solid was recrystallized from ethyl acetate-hexane to give [2-(3-{3-cyclohexyl-1-[5-(trifluoromethyl)-2-pyridinyl]-1H-pyrazol-4-yl}propoxy)-3-ethoxyphenyl]acetic acid (537 mg, yield 72%) as colorless crystals. melting point: 156-157° C.

### Example 307

To a mixture of 3-{3-cyclohexyl-1-[5-(trifluoromethyl)-2-pyridinyl]-1H-pyrazol-4-yl}-1-propanol (400 mg), methyl (2-hydroxy-3-methoxyphenyl)acetate (243 mg), tributylphosphine (563 μL) and tetrahydrofuran (100 ml) was added 1,1′-azodicarbonyldipiperidine (570 mg) at room temperature and the mixture was stirred overnight. The reaction solution was concentrated. The residue was subjected to silica gel column chromatography, and a white solid was obtained from a fraction eluted with ethyl acetate-hexane (1:12, volume ratio). A mixture of the obtained solid, 1N aqueous sodium hydroxide solution (25 ml), tetrahydrofuran (25 ml) and ethanol (25 ml) was stirred overnight at room temperature. 1N Hydrochloric acid (25 ml) was added, and the mixture was extracted with ethyl acetate. The ethyl acetate layer was washed with saturated aqueous sodium chloride solution, dried (MgSO₄) and concentrated. The obtained white solid was recrystallized from ethyl acetate-hexane to give [2-(3-{3-cyclohexyl-1-[5-(trifluoromethyl)-2-pyridinyl]-1H-pyrazol-4-yl}propoxy)-3-methoxyphenyl]acetic acid (427 mg, yield 73%) as colorless crystals. melting point: 140-141° C.

### Example 308

A mixture of methyl (2-{3-[1-(5-chloro-2-pyridinyl)-3-isopropyl-1H-pyrazol-4-yl]propoxy}phenyl)acetate (230 mg), 1N aqueous sodium hydroxide solution (20 ml), tetrahydrofuran (20 ml) and ethanol (20 ml) was stirred overnight at room temperature. 1N Hydrochloric acid (20 ml) was added, and the mixture was extracted with ethyl acetate. The ethyl acetate layer was washed with saturated aqueous sodium chloride solution, dried (MgSO₄) and concentrated. The obtained white solid was recrystallized from ethyl acetate-hexane to give (2-{3-[1-(5-chloro-2-pyridinyl)-3-isopropyl-1H-pyrazol-4-yl]propoxy}phenyl)acetic acid (182 mg, yield 82%) as colorless crystals. melting point: 141-142° C.

### Example 309

A mixture of methyl (2-{3-[1-(5-chloro-2-pyridinyl)-3-isopropyl-1H-pyrazol-4-yl]propoxy}phenyl)acetate (240 mg), 5% palladium-carbon (100 mg) and ethanol (50 ml) was stirred at room temperature for 2 days under a hydrogen atmosphere. Palladium-carbon was removed by filtration and the filtrate was concentrated. The residue was subjected to silica gel column chromatography, and a colorless oil was obtained from a fraction eluted with ethyl acetate-hexane (1:5, volume ratio). A mixture of the obtained oily substance, 1N aqueous sodium hydroxide solution (20 ml), tetrahydrofuran (20 ml) and ethanol (20 ml) was stirred overnight at room temperature. 1N Hydrochloric acid (20 ml) was added, and the mixture was extracted with ethyl acetate. The ethyl acetate layer was washed with saturated aqueous sodium chloride solution, dried (MgSO₄) and concentrated. The obtained white solid was recrystallized from ethyl acetate-hexane to give (2-{3-[3-isopropyl-1-(2-pyridinyl)-1H-pyrazol-4-yl]propoxy}phenyl)acetic acid (92.4 mg, yield 43%) as colorless crystals. melting point: 140-142° C.

### Example 310

To a mixture of 3-{3-isopropyl-1-[5-(trifluoromethyl)-2-pyridinyl]-1H-pyrazol-4-yl}-1-propanol (354 mg), methyl 3-(3-hydroxy-1-methyl-1H-pyrazol-4-yl)propanoate (246 mg), tributylphosphine (563 μL) and tetrahydrofuran (100 ml) was added 1,1′-azodicarbonyldipiperidine (570 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (25 ml), tetrahydrofuran (25 ml) and ethanol (25 ml) was stirred overnight at room temperature. 1N Hydrochloric acid (25 ml) was added, and the mixture was extracted with ethyl acetate. The ethyl acetate layer was washed with saturated aqueous sodium chloride solution, dried (MgSO₄) and concentrated. The obtained white solid was recrystallized from ethyl acetate-hexane to give 3-[3-(3-{3-isopropyl-1-[5-(trifluoromethyl)-2-pyridinyl]-1H-pyrazol-4-yl}propoxy)-1-methyl-1H-pyrazol-4-yl]propanoic acid (307 mg, yield 58%) as colorless crystals. melting point: 124-125° C.

### Example 311

A mixture of methyl (2-{3-[1-(5-chloro-2-pyridinyl)-3-isopropyl-1H-pyrazol-4-yl]propoxy}-3-methoxyphenyl)acetate (260 mg), 1N aqueous sodium hydroxide solution (20 ml), tetrahydrofuran (20 ml) and ethanol (20 ml) was stirred overnight at room temperature. 1N Hydrochloric acid (20 ml) was added, and the mixture was extracted with ethyl acetate. The ethyl acetate layer was washed with saturated aqueous sodium chloride solution, dried (MgSO₄) and concentrated. The obtained white solid was recrystallized from ethyl acetate-hexane to give (2-{3-[1-(5-chloro-2-pyridinyl)-3-isopropyl-1H-pyrazol-4-yl]propoxy}-3-methoxyphenyl)acetic acid (214 mg, yield 85%) as colorless crystals. melting point: 148-149° C.

### Example 312

A mixture of methyl (2-{3-[1-(5-chloro-2-pyridinyl)-3-isopropyl-1H-pyrazol-4-yl]propoxy}-3-methoxyphenyl)acetate (290 mg), 5% palladium-carbon (300 mg) and ethanol (50 ml) was stirred overnight at room temperature under a hydrogen atmosphere. Palladium-carbon was removed by filtration and the filtrate was concentrated. The obtained residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (20 ml), tetrahydrofuran (20 ml) and ethanol (20 ml) was stirred overnight at room temperature. 1N Hydrochloric acid (20 ml) was added, and the mixture was extracted with ethyl acetate. The ethyl acetate layer was washed with saturated aqueous sodium chloride solution, dried (MgSO₄) and concentrated. The obtained colorless oil was recrystallized from diisopropyl ether-hexane to give (2-{3-[3-isopropyl-1-(2-pyridinyl)-1H-pyrazol-4-yl]propoxy}-3-methoxyphenyl)acetic acid (82.0 mg, yield 32%) as colorless crystals. melting point: 102-104° C.

### Example 313

To a mixture of 3-{3-isopropyl-1-[5-(trifluoromethyl)-2-pyridinyl]-1H-pyrazol-4-yl}-1-propanol (170 mg), methyl (1-ethyl-3-hydroxy-1H-pyrazol-4-yl)acetate (100 mg), tributylphosphine (272 μL) and tetrahydrofuran (50 ml) was added 1,1′-azodicarbonyldipiperidine (275 mg) at room temperature and the mixture was stirred overnight. The reaction solution was concentrated. The residue was subjected to silica gel column chromatography, and a pale-yellow oily substance was obtained from a fraction eluted with ethyl acetate-hexane (1:3, volume ratio). A mixture of the obtained oily substance, 1N aqueous sodium hydroxide solution (20 ml), tetrahydrofuran (20 ml) and ethanol (20 ml) was stirred at room temperature for 4 hours. 1N Hydrochloric acid (20 ml) was added, and the mixture was extracted with ethyl acetate. The ethyl acetate layer was washed with saturated aqueous sodium chloride solution, dried (MgSO₄) and concentrated. The residue was subjected to silica gel column chromatography, and a white solid was obtained from a fraction eluted with ethyl acetate-hexane (3:2, volume ratio). The obtained solid was recrystallized from diisopropyl ether-hexane to give [1-ethyl-3-(3-{3-isopropyl-1-[5-(trifluoromethyl)-2-pyridinyl]-1H-pyrazol-4-yl}propoxy)-1H-pyrazol-4-yl]acetic acid (105 mg, yield 42%) as colorless crystals. melting point: 99-100° C.

### Example 314

To a mixture of 3-{3-propyl-1-[5-(trifluoromethyl)-2-pyridinyl]-1H-pyrazol-4-yl}-1-propanol (400 mg), methyl 3-(3-hydroxy-1-methyl-1H-pyrazol-4-yl)propanoate (278 mg), tributylphosphine (638 μL) and tetrahydrofuran (100 ml) was added 1,1′-azodicarbonyldipiperidine (646 mg) at room temperature and the mixture was stirred overnight. The reaction solution was concentrated. The residue was subjected to silica gel column chromatography, and a colorless oil was obtained from a fraction eluted with ethyl acetate-hexane (3:7, volume ratio). A mixture of the obtained oily substance, 1N aqueous sodium hydroxide solution (25 ml), tetrahydrofuran (25 ml) and ethanol (25 ml) was stirred at room temperature for 5 hours. 1N Hydrochloric acid (25 ml) was added, and the mixture was extracted with ethyl acetate. The ethyl acetate layer was washed with saturated aqueous sodium chloride solution, dried (MgSO₄) and concentrated. The obtained white solid was recrystallized from ethyl acetate-hexane to give 3-[1-methyl-3-(3-{3-propyl-1-[5-(trifluoromethyl)-2-pyridinyl]-1H-pyrazol-4-yl}propoxy)-1H-pyrazol-4-yl]propanoic acid (436 mg, yield 73%) as colorless crystals. melting point: 103-104° C.

### Example 315

To a mixture of 3-{3-isopropyl-1-[5-(trifluoromethyl)-2-pyridinyl]-1H-pyrazol-4-yl}-1-propanol (313 mg), methyl (2-hydroxy-3-methylphenyl)acetate (541 mg), tributylphosphine (748 μL) and tetrahydrofuran (100 ml) was added 1,1′-azodicarbonyldipiperidine (757 mg) at room temperature and the mixture was heated to 50° C. The mixture was stirred at said temperature for 1 hour. The reaction solution was concentrated. The residue was subjected to silica gel column chromatography, and a white solid was obtained from a fraction eluted with ethyl acetate-hexane (1:19, volume ratio). A mixture of the obtained solid, 1N aqueous sodium hydroxide solution (25 ml), tetrahydrofuran (25 ml) and ethanol (25 ml) was stirred overnight at room temperature. 1N Hydrochloric acid (25 ml) was added, and the mixture was extracted with ethyl acetate. The ethyl acetate layer was washed with saturated aqueous sodium chloride solution, dried (MgSO₄) and concentrated. The residue was subjected to silica gel column chromatography, and a white solid was obtained from a fraction eluted with ethyl acetate-hexane (7:13, volume ratio). The obtained solid was recrystallized from ethyl acetate-hexane to give [2-(3-{3-isopropyl-1-[5-(trifluoromethyl)-2-pyridinyl]-1H-pyrazol-4-yl}propoxy)-3-methylphenyl]acetic acid (135 mg, yield 29%) as colorless crystals. melting point: 159-160° C.

### Example 316

To a mixture of 3-{3-cyclohexyl-1-[5-(trifluoromethyl)-2-pyridinyl]-1H-pyrazol-4-yl}-1-propanol (500 mg), methyl (2-hydroxy-3-methylphenyl)acetate (305 mg), tributylphosphine (703 μL) and tetrahydrofuran (100 ml) was added 1,1′-azodicarbonyldipiperidine (712 mg) at room temperature and the mixture was heated to 50° C. The mixture was stirred overnight at said temperature. The reaction solution was concentrated. The residue was subjected to silica gel column chromatography, and a pale yellow oily substance was obtained from a fraction eluted with ethyl acetate-hexane (1:9, volume ratio). A mixture of the obtained oily substance, 1N aqueous sodium hydroxide solution (25 ml), tetrahydrofuran (25 ml) and ethanol (25 ml) was stirred overnight at room temperature. 1N Hydrochloric acid (25 ml) was added, and the mixture was extracted with ethyl acetate. The ethyl acetate layer was washed with saturated aqueous sodium chloride solution, dried (MgSO₄) and concentrated. The residue was subjected to silica gel column chromatography, and a white solid was obtained from a fraction eluted with ethyl acetate-hexane (3:7, volume ratio). The obtained solid was recrystallized from ethyl acetate-hexane to give [2-(3-{3-cyclohexyl-1-[5-(trifluoromethyl)-2-pyridinyl]-1H-pyrazol-4-yl}propoxy)-3-methylphenyl]acetic acid (193 mg, yield 27%) as colorless crystals. melting point: 184-185° C.

### Example 317

To a mixture of 3-{3-isopropyl-1-[5-(trifluoromethyl)-2-pyridinyl]-1H-pyrazol-4-yl}-1-propanol (329 mg), ethyl (1-cyclohexyl-3-hydroxy-1H-pyrazol-4-yl)acetate (250 mg), tributylphosphine (523 μL) and tetrahydrofuran (100 ml) was added 1,1′-azodicarbonyldipiperidine (530 mg) at room temperature and the mixture was heated to 50° C. The mixture was stirred overnight at said temperature. The reaction solution was concentrated. The residue was subjected to silica gel column chromatography, and a yellow oily substance was obtained from a fraction eluted with ethyl acetate-hexane (1:3, volume ratio). A mixture of the obtained oily substance, 1N aqueous sodium hydroxide solution (25 ml), tetrahydrofuran (25 ml) and ethanol (25 ml) was stirred overnight at room temperature. 1N Hydrochloric acid (25 ml) was added, and the mixture was extracted with ethyl acetate. The ethyl acetate layer was washed with saturated aqueous sodium chloride solution, dried (MgSO₄) and concentrated. The residue was subjected to silica gel column chromatography, and a yellow oily substance was obtained from a fraction eluted with ethyl acetate-hexane (1:1, volume ratio). The obtained oily substance was recrystallized from ethyl acetate-hexane to give [1-cyclohexyl-3-(3-{3-isopropyl-1-[5-(trifluoromethyl)-2-pyridinyl]-1H-pyrazol-4-yl}propoxy)-1H-pyrazol-4-yl]acetic acid (145 mg, yield 27%) as colorless crystals. melting point: 116-117° C.

### Example 318

To a mixture of 3-{3-isopropyl-1-[5-(trifluoromethyl)-2-pyridinyl]-1H-pyrazol-4-yl}-1-propanol (450 mg), ethyl (5-hydroxy-1-methyl-1H-pyrazol-4-yl)acetate (292 mg), tributylphosphine (716 μL) and tetrahydrofuran (100 ml) was added 1,1′-azodicarbonyldipiperidine (727 mg) at room temperature and the mixture was heated to 50° C. The mixture was stirred overnight at said temperature. The reaction solution was concentrated. The residue was subjected to silica gel column chromatography, and a colorless oil was obtained from a fraction eluted with ethyl acetate-hexane (3:7, volume ratio). A mixture of the obtained oily substance, 1N aqueous sodium hydroxide solution (25 ml), tetrahydrofuran (25 ml) and ethanol (25 ml) was stirred overnight at room temperature. 1N Hydrochloric acid (25 ml) was added, and the mixture was extracted with ethyl acetate. The ethyl acetate layer was washed with saturated aqueous sodium chloride solution, dried (MgSO₄) and concentrated. The obtained white solid was recrystallized from ethyl acetate-hexane to give [5-(3-{3-isopropyl-1-[5-(trifluoromethyl)-2-pyridinyl]-1H-pyrazol-4-yl}propoxy)-1-methyl-1H-pyrazol-4-yl]acetic acid (246 mg, yield 38%) as colorless crystals. melting point: 146-148° C.

### Example 319

To a mixture of 3-{3-propyl-1-[5-(trifluoromethyl)-2-pyridinyl]-1H-pyrazol-4-yl}-1-propanol (500 mg), methyl (2-hydroxy-3-methylphenyl)acetate (344 mg), tributylphosphine (792 μL) and tetrahydrofuran (100 ml) was added 1,1′-azodicarbonyldipiperidine (802 mg) at room temperature and the mixture was heated to 50° C. The mixture was stirred overnight at said temperature. The reaction solution was concentrated. The residue was subjected to silica gel column chromatography, and a pale yellow solid was obtained from a fraction eluted with ethyl acetate-hexane (1:9, volume ratio). A mixture of the obtained solid, 1N aqueous sodium hydroxide solution (25 ml), tetrahydrofuran (25 ml) and ethanol (25 ml) was stirred overnight at room temperature. 1N Hydrochloric acid (25 ml) was added, and the mixture was extracted with ethyl acetate. The ethyl acetate layer was washed with saturated aqueous sodium chloride solution, dried (MgSO₄) and concentrated. The obtained white solid was recrystallized from ethyl acetate-hexane to give [3-methyl-2-(3-{3-propyl-1-[5-(trifluoromethyl)-2-pyridinyl]-1H-pyrazol-4-yl}propoxy)phenyl]acetic acid (268 mg, yield 37%) as colorless crystals. melting point: 134-135° C.

### Example 320

To a mixture of 3-{3-propyl-1-[5-(trifluoromethyl)-2-pyridinyl]-1H-pyrazol-4-yl}-1-propanol (400 mg), ethyl (1-cyclohexyl-3-hydroxy-1H-pyrazol-4-yl)acetate (336 mg), tributylphosphine (638 μL) and tetrahydrofuran (100 ml) was added 1,1′-azodicarbonyldipiperidine (646 mg) at room temperature and the mixture was heated to 50° C. The mixture was stirred overnight at said temperature. The reaction solution was concentrated. The residue was subjected to silica gel column chromatography, and a colorless oil was obtained from a fraction eluted with ethyl acetate-hexane (1:3, volume ratio). A mixture of the obtained oily substance, 1N aqueous sodium hydroxide solution (25 ml), tetrahydrofuran (25 ml) and ethanol (25 ml) was stirred overnight at room temperature. 1N Hydrochloric acid (25 ml) was added, and the mixture was extracted with ethyl acetate. The ethyl acetate layer was washed with saturated aqueous sodium chloride solution, dried (MgSO₄) and concentrated. The residue was subjected to silica gel column chromatography, and a pale yellow solid was obtained from a fraction eluted with ethyl acetate-hexane (1:1, volume ratio). The obtained solid was recrystallized from ethyl acetate-hexane to give [1-cyclohexyl-3-(3-{3-propyl-1-[5-(trifluoromethyl)-2-pyridinyl]-1H-pyrazol-4-yl}propoxy)-1H-pyrazol-4-yl]acetic acid (453 mg, yield 68%) as colorless crystals. melting point: 127-128° C.

### Example 321

To a mixture of 3-{3-propyl-1-[5-(trifluoromethyl)-2-pyridinyl]-1H-pyrazol-4-yl}-1-propanol (450 mg), ethyl (5-hydroxy-1-methyl-1H-pyrazol-4-yl)acetate (291 mg), tributylphosphine (718 μL) and tetrahydrofuran (100 ml) was added 1,1′-azodicarbonyldipiperidine (727 mg) at room temperature and the mixture was heated to 50° C. The mixture was stirred overnight at said temperature. The reaction solution was concentrated. The residue was subjected to silica gel column chromatography, and a pale yellow oily substance was obtained from a fraction eluted with ethyl acetate-hexane (3:7, volume ratio). A mixture of the obtained oily substance, 1N aqueous sodium hydroxide solution (25 ml), tetrahydrofuran (25 ml) and ethanol (25 ml) was stirred overnight at room temperature. 1N Hydrochloric acid (25 ml) was added, and the mixture was extracted with ethyl acetate. The ethyl acetate layer was washed with saturated aqueous sodium chloride solution, dried (MgSO₄) and concentrated. The obtained white solid was recrystallized from ethyl acetate-hexane to give [1-methyl-5-(3-{3-propyl-1-[5-(trifluoromethyl)-2-pyridinyl]-1H-pyrazol-4-yl}propoxy)-1H-pyrazol-4-yl]acetic acid (274 mg, yield 42%) as colorless crystals. melting point: 136-138° C.

### Example 322

To a mixture of 3-{3-isopropyl-1-[5-(trifluoromethyl)-2-pyridinyl]-1H-pyrazol-4-yl}-1-propanol (500 mg), ethyl (1-ethyl-5-hydroxy-1H-pyrazol-4-yl)acetate (381 mg), tributylphosphine (797 μL) and tetrahydrofuran (100 ml) was added 1,1′-azodicarbonyldipiperidine (807 mg) at room temperature and the mixture was heated to 50° C. The mixture was stirred overnight at said temperature. The reaction solution was concentrated. The residue was subjected to silica gel column chromatography, and a pale-yellow oily substance as obtained from a fraction eluted with ethyl acetate-hexane (1:3, volume ratio). A mixture of the obtained oily substance, 1N aqueous sodium hydroxide solution (25 ml), tetrahydrofuran (25 ml) and ethanol (25 ml) was stirred overnight at room temperature. 1N Hydrochloric acid (25 ml) was added, and the mixture was extracted with ethyl acetate. The ethyl acetate layer was washed with saturated aqueous sodium chloride solution, dried (MgSO₄) and concentrated. The residue was subjected to silica gel column chromatography, and a white solid was obtained from a fraction eluted with ethyl acetate-hexane (1:1, volume ratio). The obtained solid was recrystallized from ethyl acetate-hexane to give [1-ethyl-5-(3-{3-isopropyl-1-[5-(trifluoromethyl)-2-pyridinyl]-1H-pyrazol-4-yl}propoxy)-1H-pyrazol-4-yl]acetic acid (187 mg, yield 25%) as colorless crystals. melting point: 120-121° C.

### Example 323

To a mixture of 3-{3-isopropyl-1-[5-(trifluoromethyl)-2-pyridinyl]-1H-pyrazol-4-yl}-1-propanol (500 mg), ethyl 3-(2-hydroxy-3-methoxyphenyl)propanoate (395 mg), tributylphosphine (797 μL) and tetrahydrofuran (100 ml) was added 1,1′-azodicarbonyldipiperidine (807 mg) at room temperature and the mixture was heated to 50° C. The mixture was stirred overnight at said temperature. The reaction solution was concentrated. The residue was subjected to silica gel column chromatography, and a colorless oil was obtained from a fraction eluted with ethyl acetate-hexane (1:9, volume ratio). A mixture of the obtained oily substance, 1N aqueous sodium hydroxide solution (25 ml), tetrahydrofuran (25 ml) and ethanol (25 ml) was stirred overnight at room temperature. 1N Hydrochloric acid (25 ml) was added, and the mixture was extracted with ethyl acetate. The ethyl acetate layer was washed with saturated aqueous sodium chloride solution, dried (MgSO₄) and concentrated. The obtained white solid was recrystallized from ethyl acetate-hexane to give 3-[2-(3-{3-isopropyl-1-[5-(trifluoromethyl)-2-pyridinyl]-1H-pyrazol-4-yl}propoxy)-3-methoxyphenyl]propanoic acid (429 mg, yield 55%) as colorless crystals. melting point: 112-113° C.

### Example 324

To a mixture of 3-{3-isopropyl-1-[5-(trifluoromethyl)-2-pyridinyl]-1H-pyrazol-4-yl}-1-propanol (500 mg), ethyl 3-(3-hydroxy-4-methoxyphenyl)propanoate (395 mg), tributylphosphine (797 μL) and tetrahydrofuran (100 ml) was added 1,1′-azodicarbonyldipiperidine (807 mg) at room temperature and the mixture was heated to 50° C. The mixture was stirred overnight at said temperature. The reaction solution was concentrated. The residue was subjected to silica gel column chromatography, and a colorless oil was obtained from a fraction eluted with ethyl acetate-hexane (1:9, volume ratio). A mixture of the obtained oily substance, 1N aqueous sodium hydroxide solution (25 ml), tetrahydrofuran (25 ml) and ethanol (25 ml) was stirred overnight at room temperature. 1N Hydrochloric acid (25 ml) was added, and the mixture was extracted with ethyl acetate. The ethyl acetate layer was washed with saturated aqueous sodium chloride solution, dried (MgSO₄) and concentrated. The obtained white solid was recrystallized from ethyl acetate-hexane to give 3-[3-(3-{3-isopropyl-1-[5-(trifluoromethyl)-2-pyridinyl]-1H-pyrazol-4-yl}propoxy)-4-methoxyphenyl]propanoic acid (447 mg, yield 57%) as colorless crystals. melting point: 93-94° C.

### Example 325

To a mixture of 3-{3-cyclohexyl-1-[5-(trifluoromethyl)-2-pyridinyl]-1H-pyrazol-4-yl}-1-propanol (500 mg), ethyl (5-hydroxy-1-methyl-1H-pyrazol-4-yl)acetate (286 mg), tributylphosphine (703 μL) and tetrahydrofuran (100 ml) was added 1,1′-azodicarbonyldipiperidine (712 mg) at room temperature and the mixture was heated to 50° C. The mixture was stirred overnight at said temperature. The reaction solution was concentrated. The residue was subjected to silica gel column chromatography, and a white solid was obtained from a fraction eluted with ethyl acetate-hexane (3:7, volume ratio). A mixture of the obtained solid, 1N aqueous sodium hydroxide solution (25 ml), tetrahydrofuran (25 ml) and ethanol (25 ml) was stirred overnight at room temperature. 1N Hydrochloric acid (25 ml) was added, and the mixture was extracted with ethyl acetate. The ethyl acetate layer was washed with saturated aqueous sodium chloride solution, dried (MgSO₄) and concentrated. The obtained pale-yellow crystals were recrystallized from ethyl acetate-hexane to give [5-(3-{3-cyclohexyl-1-[5-(trifluoromethyl)-2-pyridinyl]-1H-pyrazol-4-yl}propoxy)-1-methyl-1H-pyrazol-4-yl]acetic acid (538 mg, yield 78%) as colorless crystals. melting point: 137-138° C.

### Example 326

To a mixture of 3-{3-propyl-1-[5-(trifluoromethyl)-2-pyridinyl]-1H-pyrazol-4-yl}-1-propanol (500 mg), ethyl (1-ethyl-5-hydroxy-1H-pyrazol-4-yl)acetate (349 mg), tributylphosphine (797 μL) and tetrahydrofuran (100 ml) was added 1,1′-azodicarbonyldipiperidine (807 mg) at room temperature and the mixture was heated to 50° C. The mixture was stirred overnight at said temperature. The reaction solution was concentrated. The residue was subjected to silica gel column chromatography, and a yellow oily substance was obtained from a fraction eluted with ethyl acetate-hexane (1:3, volume ratio). A mixture of the obtained oily substance, 1N aqueous sodium hydroxide solution (25 ml), tetrahydrofuran (25 ml) and ethanol (25 ml) was stirred overnight at room temperature. 1N Hydrochloric acid (25 ml) was added, and the mixture was extracted with ethyl acetate. The ethyl acetate layer was washed with saturated aqueous sodium chloride solution, dried (MgSO₄) and concentrated. The obtained pale-yellow crystals were recrystallized from ethyl acetate-hexane to give [1-ethyl-5-(3-{3-propyl-1-[5-(trifluoromethyl)-2-pyridinyl]-1H-pyrazol-4-yl}propoxy)-1H-pyrazol-4-yl]acetic acid (431 mg, yield 58%) as colorless crystals. melting point: 125-126° C.

### Example 327

To a mixture of 3-{3-isopropyl-1-[5-(trifluoromethyl)-2-pyridinyl]-1H-pyrazol-4-yl}-1-propanol (400 mg), ethyl (5-hydroxy-4-methyl-1H-pyrazol-1-yl)acetate (240 mg), tributylphosphine (550 mg) and tetrahydrofuran (30 ml) was added 1,1′-azodicarbonyldipiperidine (690 mg) at room temperature and the mixture was heated to 50° C. The mixture was stirred overnight at said temperature. The reaction solution was concentrated. The residue was subjected to silica gel column chromatography, and a colorless oil was obtained from a fraction eluted with ethyl acetate-hexane (1:3, volume ratio). A mixture of the obtained oily substance, 1N aqueous sodium hydroxide solution (25 ml), tetrahydrofuran (25 ml) and ethanol (25 ml) was stirred at room temperature for 1 hour. 1N Hydrochloric acid (25 ml) was added, and the mixture was extracted with ethyl acetate. The ethyl acetate layer was washed with saturated aqueous sodium chloride solution, dried (MgSO₄) and concentrated. The residue was subjected to silica gel column chromatography, and a brown solid was obtained from a fraction eluted with methanol-ethyl acetate (1:19, volume ratio). The obtained solid was recrystallized from ethyl acetate-hexane to give [5-(3-{3-isopropyl-1-[5-(trifluoromethyl)-2-pyridinyl]-1H-pyrazol-4-yl}propoxy)-4-methyl-1H-pyrazol-1-yl]acetic acid (222 mg, yield 37%) as colorless crystals. melting point: 123-124° C.

### Example 328

To a mixture of 3-{3-isopropyl-1-[5-(trifluoromethyl)-2-pyridinyl]-1H-pyrazol-4-yl}-1-propanol (500 mg), ethyl (4-ethyl-5-hydroxy-1H-pyrazol-1-yl)acetate (349 mg), tributylphosphine (797 μL) and tetrahydrofuran (100 ml) was added 1,1′-azodicarbonyldipiperidine (807 mg) at room temperature and the mixture was heated to 50° C. The mixture was stirred overnight at said temperature. The reaction solution was concentrated. The residue was subjected to silica gel column chromatography, and a white solid was obtained from a fraction eluted with ethyl acetate-hexane (1:3, volume ratio). A mixture of the obtained solid, 1N aqueous sodium hydroxide solution (25 ml), tetrahydrofuran (25 ml) and ethanol (25 ml) was stirred at room temperature for 30 minutes. 1N Hydrochloric acid (25 ml) was added, and the mixture was extracted with ethyl acetate. The ethyl acetate layer was washed with saturated aqueous sodium chloride solution, dried (MgSO₄) and concentrated. The obtained white solid was recrystallized from ethyl acetate-hexane to give [4-ethyl-5-(3-{3-isopropyl-1-[5-(trifluoromethyl)-2-pyridinyl]-1H-pyrazol-4-yl}propoxy)-1H-pyrazol-1-yl]acetic acid (635 mg, yield 85%) as colorless crystals. melting point: 111-112° C.

### Example 329

To a mixture of 3-{3-propyl-1-[5-(trifluoromethyl)-2-pyridinyl]-1H-pyrazol-4-yl}-1-propanol (400 mg), ethyl (5-hydroxy-4-methyl-1H-pyrazol-1-yl)acetate (240 mg), tributylphosphine (550 mg) and tetrahydrofuran (30 ml) was added 1,1′-azodicarbonyldipiperidine (690 mg) at room temperature and the mixture was heated to 50° C. The mixture was stirred overnight at said temperature. The reaction solution was concentrated. The residue was subjected to silica gel column chromatography, and a colorless oil was obtained from a fraction eluted with ethyl acetate-hexane (1:3, volume ratio). A mixture of the obtained oily substance, 1N aqueous sodium hydroxide solution (25 ml), tetrahydrofuran (25 ml) and ethanol (25 ml) was stirred at room temperature for 1 hour. 1N Hydrochloric acid (25 ml) was added, and the mixture was extracted with ethyl acetate. The ethyl acetate layer was washed with saturated aqueous sodium chloride solution, dried (MgSO₄) and concentrated. The obtained white solid was recrystallized from ethyl acetate-hexane to give [4-methyl-5-(3-{3-propyl-1-[5-(trifluoromethyl)-2-pyridinyl]-1H-pyrazol-4-yl}propoxy)-1H-pyrazol-1-yl]acetic acid (433 mg, yield 75%) as colorless crystals. melting point: 145-146° C.

### Example 330

To a mixture of 3-{3-(1-ethylpropyl)-1-[5-(trifluoromethyl)-2-pyridinyl]-1H-pyrazol-4-yl}-1-propanol (500 mg), ethyl (4-ethyl-5-hydroxy-1H-pyrazol-1-yl)acetate (318 mg), tributylphosphine (728 μL) and tetrahydrofuran (100 ml) was added 1,1′-azodicarbonyldipiperidine (737 mg) at room temperature and the mixture was heated to 50° C. The mixture was stirred overnight at said temperature. The reaction solution was concentrated. The residue was subjected to silica gel column chromatography, and a yellow oily substance was obtained from a fraction eluted with ethyl acetate-hexane (1:3, volume ratio). A mixture of the obtained oily substance, 1N aqueous sodium hydroxide solution (25 ml), tetrahydrofuran (25 ml) and ethanol (25 ml) was stirred at room temperature for 30 minutes. 1N Hydrochloric acid (25 ml) was added, and the mixture was extracted with ethyl acetate. The ethyl acetate layer was washed with saturated aqueous sodium chloride solution, dried (MgSO₄) and concentrated. The obtained colorless oil was recrystallized from ethyl acetate-hexane to give [4-ethyl-5-(3-{3-(1-ethylpropyl)-1-[5-(trifluoromethyl)-2-pyridinyl]-1H-pyrazol-4-yl}propoxy)-1H-pyrazol-1-yl]acetic acid (389 mg, yield 54%) as colorless crystals. melting point: 124-125° C.

### Example 331

To a mixture of 3-{3-propyl-1-[5-(trifluoromethyl)-2-pyridinyl]-1H-pyrazol-4-yl}-1-propanol (400 mg), ethyl (4-ethyl-5-hydroxy-1H-pyrazol-1-yl)acetate (279 mg), tributylphosphine (638 μL) and tetrahydrofuran (100 ml) was added 1,1′-azodicarbonyldipiperidine (646 mg) at room temperature and the mixture was heated to 50° C. The mixture was stirred at said temperature for 8 hours. The reaction solution was concentrated. The residue was subjected to silica gel column chromatography, and a white solid was obtained from a fraction eluted with ethyl acetate-hexane (1:3, volume ratio). A mixture of the obtained solid, 1N aqueous sodium hydroxide solution (25 ml), tetrahydrofuran (25 ml) and ethanol (25 ml) was stirred at room temperature for 1 hour. 1N Hydrochloric acid (25 ml) was added, and the mixture was extracted with ethyl acetate. The ethyl acetate layer was washed with saturated aqueous sodium chloride solution, dried (MgSO₄) and concentrated. The obtained white solid was recrystallized from ethyl acetate-hexane to give [4-ethyl-5-(3-{3-propyl-1-[5-(trifluoromethyl)-2-pyridinyl]-1H-pyrazol-4-yl}propoxy)-1H-pyrazol-1-yl]acetic acid (420 mg, yield 71%) as colorless crystals. melting point: 135-136° C.

### Example 332

To a mixture of 1-{6-[4-(3-hydroxypropyl)-3-isopropyl-1H-pyrazol-1-yl]-3-pyridinyl}ethanone (320 mg), methyl (2-hydroxy-3-methoxyphenyl)acetate (239 mg), tributylphosphine (553 μL) and tetrahydrofuran (100 ml) was added 1,1′-azodicarbonyldipiperidine (560 mg) at room temperature and the mixture was stirred for 3 hours. The reaction solution was concentrated. The residue was subjected to silica gel column chromatography, and a yellow oily substance was obtained from a fraction eluted with ethyl acetate-hexane (1:3, volume ratio). A mixture of the obtained oily substance, 1N aqueous sodium hydroxide solution (25 ml), tetrahydrofuran (25 ml) and ethanol (25 ml) was stirred at room temperature for 1 hour. 1N Hydrochloric acid (25 ml) was added, and the mixture was extracted with ethyl acetate. The ethyl acetate layer was washed with saturated aqueous sodium chloride solution, dried (MgSO₄) and concentrated. The obtained white solid was recrystallized from ethyl acetate-hexane to give (2-{3-[1-(5-acetyl-2-pyridinyl)-3-isopropyl-1H-pyrazol-4-yl]propoxy}-3-methoxyphenyl)acetic acid (299 mg, yield 60%) as colorless crystals. melting point: 148-149° C.

### Example 333

To a mixture of 3-{3-(1-ethylpropyl)-1-[5-(trifluoromethyl)-2-pyridinyl]-1H-pyrazol-4-yl}-1-propanol (500 mg), methyl (1-ethyl-3-hydroxy-1H-pyrazol-4-yl)acetate (297 mg), tributylphosphine (728 μL) and tetrahydrofuran (100 ml) was added 1,1′-azodicarbonyldipiperidine (737 mg) at room temperature and the mixture was stirred overnight. The reaction solution was concentrated. The residue was subjected to silica gel column chromatography, and a colorless oil was obtained from a fraction eluted with ethyl acetate-hexane (1:5, volume ratio). A mixture of the obtained oily substance, 1N aqueous sodium hydroxide solution (25 ml), tetrahydrofuran (25 ml) and ethanol (25 ml) was stirred at room temperature for 1 hour. 1N Hydrochloric acid (25 ml) was added, and the mixture was extracted with ethyl acetate. The ethyl acetate layer was washed with saturated aqueous sodium chloride solution, dried (MgSO₄) and concentrated. The residue was subjected to silica gel column chromatography, and [1-ethyl-3-(3-{3-(1-ethylpropyl)-1-[5-(trifluoromethyl)-2-pyridinyl]-1H-pyrazol-4-yl}propoxy)-1H-pyrazol-4-yl]acetic acid (520 mg, yield 72%) was obtained as amorphous from a fraction eluted with ethyl acetate-hexane (2:3, volume ratio).

¹H-NMR (CDCl₃) δ: 0.85 (6H, t, J=7.2 Hz), 1.40 (3H, t, J=7.2 Hz), 1.60-1.83 (4H, m), 2.01-2.13 (2H, m), 2.52-2.67 (3H, m), 3.44 (2H, s), 3.96 (2H, q, J=7.2 Hz), 4.21-4.28 (2H, m), 7.20 (1H, s), 7.90-7.96 (1H, m), 8.02 (1H, d, J=8.4 Hz), 8.31 (1H, s), 8.56-8.60 (1H, m).

### Example 334

To a mixture of 3-{3-butyl-1-[5-(trifluoromethyl)-2-pyridinyl]-1H-pyrazol-4-yl}-1-propanol (500 mg), methyl (1-ethyl-3-hydroxy-1H-pyrazol-4-yl)acetate (310 mg), tributylphosphine (762 μL) and tetrahydrofuran (100 ml) was added 1,1′-azodicarbonyldipiperidine (727 mg) at room temperature and the mixture was stirred overnight. The reaction solution was concentrated. The residue was subjected to silica gel column chromatography, and a white solid was obtained from a fraction eluted with ethyl acetate-hexane (1:3, volume ratio). A mixture of the obtained solid, 1N aqueous sodium hydroxide solution (25 ml), tetrahydrofuran (25 ml) and ethanol (25 ml) was stirred at room temperature for 1 hour. 1N Hydrochloric acid (25 ml) was added, and the mixture was extracted with ethyl acetate. The ethyl acetate layer was washed with saturated aqueous sodium chloride solution, dried (MgSO₄) and concentrated. The obtained white solid was recrystallized from ethyl acetate-hexane to give [3-(3-{3-butyl-1-[5-(trifluoromethyl)-2-pyridinyl]-1H-pyrazol-4-yl}propoxy)-1-ethyl-1H-pyrazol-4-yl]acetic acid (403 mg, yield 55%) as colorless crystals. melting point: 109-110° C.

### Example 335

To a mixture of 3-{3-ethoxy-1-[5-(trifluoromethyl)-2-pyridinyl]-1H-pyrazol-4-yl}-1-propanol (500 mg), methyl (1-ethyl-3-hydroxy-1H-pyrazol-4-yl)acetate (322 mg), tributylphosphine (792 μL) and tetrahydrofuran (100 ml) was added 1,1′-azodicarbonyldipiperidine (802 mg) at room temperature and the mixture was stirred overnight. The reaction solution was concentrated. The residue was subjected to silica gel column chromatography, and a colorless oil was obtained from a fraction eluted with ethyl acetate-hexane (1:3, volume ratio). A mixture of the obtained oily substance, 1N aqueous sodium hydroxide solution (25 ml), tetrahydrofuran (25 ml) and ethanol (25 ml) was stirred at room temperature for 1 hour. 1N Hydrochloric acid (25 ml) was added, and the mixture was extracted with ethyl acetate. The ethyl acetate layer was washed with saturated aqueous sodium chloride solution, dried (MgSO₄) and concentrated. The obtained white solid was recrystallized from ethyl acetate-hexane to give [3-(3-{3-ethoxy-1-[5-(trifluoromethyl)-2-pyridinyl]-1H-pyrazol-4-yl}propoxy)-1-ethyl-1H-pyrazol-4-yl]acetic acid (398 mg, yield 54%) as colorless crystals. melting point: 108-109° C.

### Example 336

To a mixture of 3-[1-(3,5-dichloro-2-pyridyl)-3-isopropyl-1H-pyrazol-4-yl]-1-propanol (500 mg), methyl (2-hydroxy-3-methoxyphenyl)acetate (315 mg), tributylphosphine (645 mg) and tetrahydrofuran (60 ml) was added 1,1′-azodicarbonyldipiperidine (805 mg) at room temperature and the mixture was stirred overnight. The reaction solution was concentrated and isopropyl ether (20 ml) was added to the residue. The insoluble material was removed by filtration and the filtrate was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (3 ml), tetrahydrofuran (6 ml) and methanol (6 ml) was stirred at 50° C. for 2 hours and poured into water. 2N Hydrochloric acid (3 ml) was added, and the mixture was extracted with ethyl acetate. The ethyl acetate layer was washed with saturated aqueous sodium chloride solution, dried (MgSO₄) and concentrated. The obtained colorless crystals were collected by filtration to give (2-{3-[1-(3,5-dichloro-2-pyridyl)-3-isopropyl-1H-pyrazol-4-yl]propoxy}-3-methoxyphenyl)acetic acid (550 mg, yield 72%). The crystals were recrystallized from ethyl acetate-hexane. melting point: 105-106° C.

### Example 337

To a mixture of 6-[4-(3-hydroxypropyl)-3-isopropyl-1H-pyrazol-1-yl]pyridazine-3-carbonitrile (500 mg), methyl (2-hydroxy-3-methoxyphenyl)acetate (430 mg), tributylphosphine (740 mg) and tetrahydrofuran (30 ml) was added 1,1′-azodicarbonyldipiperidine (930 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 2N hydrochloric acid (3 ml) and 1,4-dioxane (6 ml) was stirred while heating under reflux for 6 hours. Water (20 ml) was added, and the mixture was extracted with ethyl acetate. The ethyl acetate layer was washed with saturated aqueous sodium chloride solution, dried (MgSO₄) and concentrated. The residue was subjected to silica gel column chromatography, and (2-{3-[1-(6-cyanopyridazin-3-yl)-3-isopropyl-1H-pyrazol-4-yl]propoxy}-3-methoxyphenyl)acetic acid (300 mg, yield 37%) was obtained as colorless crystals from a fraction eluted with ethyl acetate-hexane (1:1, volume ratio). The crystals were recrystallized from ethyl acetate-hexane. melting point: 132-133° C.

### Example 338

To a mixture of 2-[4-(3-hydroxypropyl)-3-isopropyl-1H-pyrazol-1-yl]pyrimidine-5-carbonitrile (330 mg), methyl (2-hydroxy-3-methoxyphenyl)acetate (280 mg), tributylphosphine (490 mg) and tetrahydrofuran (30 ml) was added 1,1′-azodicarbonyldipiperidine (930 mg) at room temperature and the mixture was stirred overnight. The reaction solution was concentrated. The residue was subjected to silica gel column chromatography, and a colorless oil was obtained from a fraction eluted with ethyl acetate-hexane (1:3, volume ratio). A mixture of the obtained oily substance, 2N hydrochloric acid (3 ml) and 1,4-dioxane (6 ml) was stirred while heating under reflux for 6 hours. Water (20 ml) was added, and the mixture was extracted with ethyl acetate. The ethyl acetate layer was washed with saturated aqueous sodium chloride solution, dried (MgSO₄) and concentrated. The residue was subjected to silica gel column chromatography, and (2-{3-[1-(5-cyanopyrimidin-2-yl)-3-isopropyl-1H-pyrazol-4-yl]propoxy}-3-methoxyphenyl)acetic acid (140 mg, yield 28%) was obtained as colorless crystals from a fraction eluted with ethyl acetate-hexane (3:1, volume ratio). The crystals were recrystallized from ethyl acetate-hexane. melting point: 167-168° C.

### Example 339

To a mixture of 3-{3-(1-ethylpropyl)-1-[6-(trifluoromethyl)pyridazin-3-yl]-1H-pyrazol-4-yl}-1-propanol (700 mg), methyl (2-hydroxy-3-methoxyphenyl)acetate (480 mg), tributylphosphine (820 mg) and tetrahydrofuran (50 ml) was added 1,1′-azodicarbonyldipiperidine (1.03 g) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (4 ml), tetrahydrofuran (6 ml) and methanol (6 ml) was stirred at room temperature for hours. 1N Hydrochloric acid (4 ml) was added, and the mixture was extracted with ethyl acetate. The ethyl acetate layer was washed with saturated aqueous sodium chloride solution, dried (MgSO₄) and concentrated. The obtained colorless crystals were collected by filtration to give [2-(3-{3-(1-ethylpropyl)-1-[6-(trifluoromethyl)pyridazin-3-yl]-1H-pyrazol-4-yl}propoxy)-3-methoxyphenyl]acetic acid (760 mg, yield 76%). The crystals were recrystallized from ethyl acetate-hexane. melting point: 81-82° C.

### Example 340

To a mixture of 3-{3-(1-ethylpropyl)-1-[6-(trifluoromethyl)pyridazin-3-yl]-1H-pyrazol-4-yl}-1-propanol (700 mg), methyl (2-hydroxy-3-methylphenyl)acetate (440 mg), tributylphosphine (820 mg) and tetrahydrofuran (50 ml) was added 1,1′-azodicarbonyldipiperidine (1.03 g) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (4 ml), tetrahydrofuran (6 ml) and methanol (6 ml) was stirred at room temperature for 5 hours. 1N Hydrochloric acid (4 ml) was added, and the mixture was extracted with ethyl acetate. The ethyl acetate layer was washed with saturated aqueous sodium chloride solution, dried (MgSO₄) and concentrated. The obtained colorless crystals were collected by filtration to give [2-(3-{3-(1-ethylpropyl)-1-[6-(trifluoromethyl)pyridazin-3-yl]-1H-pyrazol-4-yl}propoxy)-3-methylphenyl]acetic acid (730 mg, yield 73%). The crystals were recrystallized from ethyl acetate-hexane. melting point: 76-77° C.

### Example 341

To a mixture of 3-{3-(1-ethylpropyl)-1-[6-(trifluoromethyl)pyridazin-3-yl]-1H-pyrazol-4-yl}-1-propanol (280 mg), methyl (3-ethyl-2-hydroxyphenyl)acetate (180 mg), tributylphosphine (330 mg) and tetrahydrofuran (30 ml) was added 1,1′-azodicarbonyldipiperidine (410 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (2 ml), tetrahydrofuran (4 ml) and methanol (4 ml) was stirred at room temperature for 5 hours. 1N Hydrochloric acid (2 ml) was added and the mixture was extracted with ethyl acetate. The ethyl acetate layer was washed with saturated aqueous sodium chloride solution, dried (MgSO₄) and concentrated. The obtained colorless crystals were collected by filtration to give [3-ethyl-2-(3-{3-(1-ethylpropyl)-1-[6-(trifluoromethyl)pyridazin-3-yl]-1H-pyrazol-4-yl}propoxy)phenyl]acetic acid (250 mg, yield 60%). The crystals were recrystallized from ethyl acetate-hexane. melting point: 93-94° C.

### Example 342

To a mixture of 3-{3-(1-ethylpropyl)-1-[5-(trifluoromethyl)pyrimidin-2-yl]-1H-pyrazol-4-yl}propanol (550 mg), methyl (2-hydroxy-3-methoxyphenyl)acetate (350 mg), tributylphosphine (710 mg) and tetrahydrofuran (30 ml) was added 1,1′-azodicarbonyldipiperidine (930 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 2N hydrochloric acid (3 ml) and 1,4-dioxane (6 ml) was stirred while heating under reflux for 6 hours. Water (20 ml) was added, and the mixture was extracted with ethyl acetate. The ethyl acetate layer was washed with saturated aqueous sodium chloride solution, dried (MgSO₄) and concentrated. The residue was subjected to silica gel column chromatography, and [2-(3-{3-(1-ethylpropyl)-1-[5-(trifluoromethyl)pyrimidin-2-yl]-1H-pyrazol-4-yl}propoxy)-3-methoxyphenyl]acetic acid (180 mg, yield 22%) was obtained as colorless crystals from a fraction eluted with ethyl acetate-hexane (1:1, volume ratio). The crystals were recrystallized from ethyl acetate-hexane. melting point: 163-164° C.

### Example 343

To a mixture of 3-{3-(1-ethylpropyl)-1-[5-(trifluoromethyl)pyrimidin-2-yl]-1H-pyrazol-4-yl}propanol (610 mg), methyl (2-hydroxy-3-methylphenyl)acetate (360 mg), tributylphosphine (720 mg) and tetrahydrofuran (30 ml) was added 1,1′-azodicarbonyldipiperidine (900 mg) at room temperature and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 2N hydrochloric acid (3 ml) and 1,4-dioxane (6 ml) was stirred while heating under reflux for 6 hours. Water (20 ml) was added, and the mixture was extracted with ethyl acetate. The ethyl acetate layer was washed with saturated aqueous sodium chloride solution, dried (MgSO₄) and concentrated. The residue was subjected to silica gel column chromatography, and [2-(3-{3-(1-ethylpropyl)-1-[5-(trifluoromethyl)pyrimidin-2-yl]-1H-pyrazol-4-yl}propoxy)-3-methylphenyl]acetic acid (260 mg, yield 30%) was obtained as colorless crystals from a fraction eluted with ethyl acetate-hexane (1:1, volume ratio). The crystals were recrystallized from ethyl acetate-hexane. melting point: 110-111° C.

### Example 344

To a mixture of 3-{3-isopropyl-1-[5-(trifluoromethyl)-1,3,4-thiadiazol-2-yl]-1H-pyrazol-4-yl}-1-propanol (400 mg), methyl (2-hydroxy-3-methoxyphenyl)acetate (270 mg), tributylphosphine (510 mg) and tetrahydrofuran (30 ml) was added 1,1′-azodicarbonyldipiperidine (630 mg) at room temperature, and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (2 ml), tetrahydrofuran (4 ml) and methanol (4 ml) was stirred at room temperature for 5 hours, and 1N hydrochloric acid (2 ml) was added. The mixture was extracted with ethyl acetate. The ethyl acetate layer was washed with saturated brine, dried (MgSO₄) and concentrated. The obtained colorless crystals were collected by filtration to give [2-(3-{3-isopropyl-1-[5-(trifluoromethyl)-1,3,4-thiadiazol-2-yl]-1H-pyrazol-4-yl}propoxy)-3-methoxyphenyl]acetic acid (131 mg, yield 22%) as colorless crystals. The crystals were recrystallized from ethyl acetate-hexane. melting point: 114-115° C.

### Example 345

To a mixture of 3-{3-tert-butyl-1-[5-(trifluoromethyl)-2-pyridinyl]-1H-pyrazol-4-yl}-1-propanol (500 mg), methyl (2-hydroxy-3-methoxyphenyl)acetate (330 mg), tributylphosphine (762 μl) and tetrahydrofuran (75 ml) was added 1,1′-azodicarbonyldipiperidine (772 mg) at room temperature, and the mixture was stirred overnight. The reaction solution was concentrated. The residue was subjected to silica gel column chromatography, and a pale-yellow oily substance was obtained from a fraction eluted with ethyl acetate-hexane (1:9, volume ratio). A mixture of the obtained oily substance, 1N aqueous sodium hydroxide solution (25 ml), tetrahydrofuran (25 ml) and ethanol (25 ml) was stirred at room temperature for 1 hour, and 1N hydrochloric acid (25 ml) was added. The mixture was extracted with ethyl acetate. The ethyl acetate layer was washed with saturated brine, dried (MgSO₄) and concentrated. The obtained colorless oil was crystallized from hexane to give [2-(3-{3-tert-butyl-1-[5-(trifluoromethyl)-2-pyridinyl]-1H-pyrazol-4-yl}propoxy)-3-methoxyphenyl]acetic acid (511 mg, yield 68%) as colorless crystals. melting point: 94-95° C.

### Example 346

To a mixture of 3-{3-tert-butyl-1-[5-(trifluoromethyl)-2-pyridinyl]-1H-pyrazol-4-yl}-1-propanol (380 mg), methyl (2-hydroxy-3-methylphenyl)acetate (230 mg), tributylphosphine (578 μl) and tetrahydrofuran (50 ml) was added 1,1′-azodicarbonyldipiperidine (585 mg) at room temperature, and the mixture was stirred overnight. The reaction solution was concentrated. The residue was subjected to silica gel column chromatography, and a yellow oily substance was obtained from a fraction eluted with ethyl acetate-hexane (1:9, volume ratio). A mixture of the obtained oily substance, 1N aqueous sodium hydroxide solution (25 ml), tetrahydrofuran (25 ml) and ethanol (25 ml) was stirred at room temperature for 1 hour, and 1N hydrochloric acid (25 ml) was added. The mixture was extracted with ethyl acetate. The ethyl acetate layer was washed with saturated brine, dried (MgSO₄) and concentrated. The obtained pale-yellow oily substance was crystallized from hexane to give [2-(3-{3-tert-butyl-1-[5-(trifluoromethyl)-2-pyridinyl]-1H-pyrazol-4-yl}propoxy)-3-methylphenyl]acetic acid (236 mg, yield 43%) as colorless crystals. melting point: 86-87° C.

### Example 347

To a mixture of 3-{3-tert-butyl-1-[5-(trifluoromethyl)-2-pyridinyl]-1H-pyrazol-4-yl}-1-propanol (500 mg), methyl (3-ethyl-2-hydroxyphenyl)acetate (326 mg), tributylphosphine (762 μl) and tetrahydrofuran (75 ml) was added 1,1′-azodicarbonyldipiperidine (772 mg) at room temperature, and the mixture was stirred overnight. The reaction solution was concentrated. The residue was subjected to silica gel column chromatography, and a yellow oily substance was obtained from a fraction eluted with ethyl acetate-hexane (1:9, volume ratio). A mixture of the obtained oily substance, 1N aqueous sodium hydroxide solution (25 ml), tetrahydrofuran (25 ml) and ethanol (25 ml) was stirred at room temperature for 3 hours and 1N hydrochloric acid (25 ml) was added. The mixture was extracted with ethyl acetate. The ethyl acetate layer was washed with saturated brine, dried (MgSO₄) and concentrated. The obtained pale-yellow oily substance was crystallized from hexane to give [2-(3-{3-tert-butyl-1-[5-(trifluoromethyl)-2-pyridinyl]-1H-pyrazol-4-yl}propoxy)-3-ethylphenyl]acetic acid (370 mg, yield 49%) as colorless crystals. melting point: 98-99° C.

### Example 348

To a mixture of 3-{3-(1-ethylpropyl)-1-[5-(trifluoromethyl)-2-pyridinyl]-1H-pyrazol-4-yl}-1-propanol (330 mg), methyl (3-hydroxy-2-methyl-4-pyridinyl)acetate (175 mg), tributylphosphine (538 μl) and tetrahydrofuran (50 ml) was added 1,1′-azodicarbonyldipiperidine (488 mg) at room temperature, and the mixture was stirred at 50° C. for 2 hours. The reaction solution was concentrated. The residue was subjected to silica gel column chromatography, and a yellow oily substance was obtained from a fraction eluted with ethyl acetate-hexane (1:4, volume ratio). A mixture of the obtained oily substance, 1N aqueous sodium hydroxide solution (15 ml), tetrahydrofuran (15 ml) and ethanol (15 ml) was stirred at room temperature for 30 minutes. 1N Hydrochloric acid (15 ml) was added, and the mixture was extracted with ethyl acetate. The ethyl acetate layer was washed with saturated brine, dried (MgSO₄) and concentrated. The residue was subjected to silica gel column chromatography, and a yellow oily substance was obtained from a fraction eluted with methanol-ethyl acetate (1:3, volume ratio). The obtained a yellow oily substance was crystallized from hexane to give [3-(3-{3-(1-ethylpropyl)-1-[5-(trifluoromethyl)-2-pyridinyl]-1H-pyrazol-4-yl}propoxy)-2-methyl-4-pyridinyl]acetic acid (53.0 mg, yield 11%) as pale-yellow crystals. melting point: 78-79° C.

### Example 349

To a mixture of 2-{3-isopropyl-1-[5-(trifluoromethyl)-2-pyridyl]-1H-pyrazol-4-yl}-1-ethanol (400 mg), methyl (2-hydroxy-3-methoxyphenyl)acetate (315 mg), tributylphosphine (540 mg) and tetrahydrofuran (30 ml) was added 1,1′-azodicarbonyldipiperidine (670 mg) at room temperature, and the mixture was stirred overnight. The reaction solution was concentrated. The residue was subjected to silica gel column chromatography, and a colorless oil was obtained from a fraction eluted with ethyl acetate-hexane (1:4, volume ratio). A mixture of the obtained oily substance, 1N aqueous sodium hydroxide solution (2 ml), tetrahydrofuran (4 ml) and methanol (4 ml) was stirred at room temperature for 5 hours. 1N Hydrochloric acid (2 ml) was added, and the mixture was extracted with ethyl acetate. The ethyl acetate layer was washed with saturated brine, dried (MgSO₄) and concentrated. The obtained colorless crystals were collected by filtration to give (2-{3-isopropyl-1-[5-(trifluoromethyl)-2-pyridyl]-1H-pyrazol-4-yl}ethoxy-3-methoxyphenyl)acetic acid (535 mg, yield 86%) as colorless crystals. The crystals were recrystallized from ethyl acetate-hexane. melting point: 165-166° C.

### Example 350

To a mixture of 3-[1-(5-bromo-2-pyridinyl)-3-(1-ethylpropyl)-1H-pyrazol-4-yl]-1-propanol (400 mg), methyl (2-hydroxy-3-methoxyphenyl)acetate (230 mg), tributylphosphine (490 mg) and tetrahydrofuran (30 ml) was added 1,1′-azodicarbonyldipiperidine (610 mg) at room temperature, and the mixture was stirred overnight at 65° C. The reaction solution was concentrated. Isopropyl ether was added and the insoluble material was filtered off. The filtrate was concentrated. The residue was subjected to silica gel column chromatography, and methyl (2-{3-[1-(5-bromo-2-pyridinyl)-3-(1-ethylpropyl)-1H-pyrazol-4-yl]propoxy}-3-methoxyphenyl)acetate (520 mg, yield 86%) was obtained as a colorless oil from a fraction eluted with ethyl acetate-hexane (1:9, volume ratio).

¹H-NMR (CDCl₃) δ: 0.87 (6H, t, J=7.6 Hz), 1.69-1.79 (4H, m), 2.02-2.09 (2H, m), 2.57-2.67 (3H, m), 3.68 (3H, s), 3.69 (2H, s), 3.85 (3H, s), 4.06 (2H, t, J=6.4 Hz), 6.82-6.87 (2H, m), 7.01 (1H, t, J=8.0 Hz), 7.83-7.84 (2H, m), 8.25 (1H, s), 8.38-8.39 (1H, m).

### Example 351

A mixture of methyl (2-{3-[1-(5-bromo-2-pyridinyl)-3-(1-ethylpropyl)-1H-pyrazol-4-yl]propoxy}-3-methoxyphenyl)acetate (500 mg), 1N aqueous sodium hydroxide solution (7 ml), tetrahydrofuran (5 ml) and methanol (7 ml) was stirred at room temperature for 4 hours and 1N hydrochloric acid (7 ml) was added. The mixture was extracted with ethyl acetate. The ethyl acetate layer was washed with saturated brine, dried (MgSO₄) and concentrated. The obtained colorless crystals were collected by filtration to give (2-{3-[1-(5-bromo-2-pyridinyl)-3-(1-ethylpropyl)-1H-pyrazol-4-yl]propoxy}-3-methoxyphenyl)acetic acid (430 mg, yield 88%). The crystals were recrystallized from hexane-ethyl acetate. melting point: 94-95° C.

### Example 352

To a mixture of 3-[1-(5-bromo-2-pyridinyl)-3-(1-ethylpropyl)-1H-pyrazol-4-yl]-1-propanol (400 mg), methyl (2-hydroxy-3-methylphenyl)acetate (210 mg), tributylphosphine (490 mg) and tetrahydrofuran (30 ml) was added 1,1′-azodicarbonyldipiperidine (610 mg) at room temperature, and the mixture was stirred overnight at 65° C. The reaction solution was concentrated. Isopropyl ether was added and the insoluble material was filtered off. The filtrate was concentrated. The residue was subjected to silica gel column chromatography, and methyl (2-{3-[1-(5-bromo-2-pyridinyl)-3-(1-ethylpropyl)-1H-pyrazol-4-yl]propoxy}-3-methylphenyl)acetate (380 mg, yield 65%) was obtained as a colorless oil from a fraction eluted with ethyl acetate-hexane (1:9, volume ratio).

¹H-NMR (CDCl₃) δ: 0.87(6H, t, J=7.2 Hz), 1.68-1.78 (4H,m), 2.07-2.13 (2H, m), 2.30 (3H, s), 2.57-2.70 (3H, m), 3.68 (2H, s), 3.68 (3H, s), 3.87 (2H, t, J=6.4 Hz), 6.99 (1H, t, J=7.6 Hz), 7.09-7.11 (2H, m), 7.84 (2H, m), 8.24 (1H, s), 8.38-8.39 (1H, m).

### Example 353

A mixture of methyl (2-{3-[1-(5-bromo-2-pyridinyl)-3-(1-ethylpropyl)-1H-pyrazol-4-yl]propoxy}-3-methylphenyl)acetate (360 mg), 1N aqueous sodium hydroxide solution (7 ml), tetrahydrofuran (5 ml) and methanol (7 ml) was stirred at room temperature for 4 hours and 1N hydrochloric acid (7 ml) was added. The mixture was extracted with ethyl acetate. The ethyl acetate layer was washed with saturated brine, dried (MgSO₄) and concentrated. The obtained colorless crystals were collected by filtration to give (2-{3-[1-(5-bromo-2-pyridinyl)-3-(1-ethylpropyl)-1H-pyrazol-4-yl]propoxy}-3-methylphenyl) acetic acid (310 mg, yield 89%). The crystals were recrystallized from hexane-ethyl acetate. melting point: 120-121° C.

### Example 354

To a mixture of 3-[1-(5-bromo-2-pyridinyl)-3-(1-ethylpropyl)-1H-pyrazol-4-yl]-1-propanol (400 mg), methyl (3-ethyl-2-hydroxyphenyl)acetate (230 mg), tributylphosphine (490 mg) and tetrahydrofuran (30 ml) was added 1,1′-azodicarbonyldipiperidine (610 mg) at room temperature, and the mixture was stirred overnight at 65° C. The reaction solution was concentrated. Isopropyl ether was added and the insoluble material was filtered off. The filtrate was concentrated. The residue was subjected to silica gel column chromatography, and methyl (2-{3-[1-(5-bromo-2-pyridinyl)-3-(1-ethylpropyl)-1H-pyrazol-4-yl]propoxy}-3-ethylphenyl)acetate (280 mg, yield 47%) was obtained as a colorless oil from a fraction eluted with ethyl acetate-hexane (1:9, volume ratio).

¹H-NMR (CDCl₃) δ: 0.88 (6H, t, J=7.6 Hz), 1.23 (3H, t, J=7.6 Hz), 1.69-1.79 (4H, m), 2.08-2.15 (2H, m), 2.58-2.70 (5H, m), 3.68 (2H, s), 3.68 (3H, s), 3.86 (2H,t, J=6.4 Hz), 7.04 (1H, t, J=7.6 Hz), 7.11 (1H, dd, J=7.6, 2.0 Hz), 7.15 (1H, dd, J=7.6, 2.0 Hz), 7.84 (2H, m), 8.24 (1H, s), 8.39 (1H, m).

### Example 355

A mixture of methyl (2-{3-[1-(5-bromo-2-pyridinyl)-3-(1-ethylpropyl)-1H-pyrazol-4-yl]propoxy}-3-ethylphenyl)acetate (220 mg), 1N aqueous sodium hydroxide solution (5 ml), tetrahydrofuran (5 ml) and methanol (5 ml) was stirred at room temperature for 4 hours. 1N Hydrochloric acid (5 ml) was added, and the mixture was extracted with ethyl acetate. The ethyl acetate layer was washed with saturated brine, dried (MgSO₄) and concentrated. The obtained colorless crystals were collected by filtration to give (2-{3-[1-(5-bromo-2-pyridinyl)-3-(1-ethylpropyl)-1H-pyrazol-4-yl]propoxy}-3-ethylphenyl)acetic acid (180 mg, yield 84%). The crystals were recrystallized from hexane-ethyl acetate. melting point: 123° C.

### Example 356

To a mixture of 3-{3-isopropyl-1-[5-(trifluoromethyl)-2-pyridinyl]-1H-pyrazol-4-yl}-1-propanol (410 mg), methyl (2-hydroxy-3-isopropylphenyl)acetate (280 mg), tributylphosphine (560 mg) and tetrahydrofuran (30 ml) was added 1,1′-azodicarbonyldipiperidine (700 mg) at room temperature, and the mixture was stirred overnight at 65° C. The reaction solution was concentrated. Isopropyl ether was added and the insoluble material was filtered off. The filtrate was concentrated. The residue was subjected to silica gel column chromatography, and methyl [3-isopropyl-2-(3-{3-isopropyl-1-[5-(trifluoromethyl)-2-pyridinyl]-1H-pyrazol-4-yl}propoxy)phenyl]acetate (430 mg, yield 66%) was obtained as a colorless oil from a fraction eluted with ethyl acetate-hexane (1:9, volume ratio).

¹H-NMR (CDCl₃) δ: 1.22 (6H, d, J=6.8 Hz), 1.36 (6H, d, J=7.2 Hz), 2.12-2.18 (2H, m), 2.74 (2H, t, J=8.0 Hz), 3.04-3.10 (1H, m), 3.25-3.31 (1H, m), 3.69 (2H, s), 3.69 (3H, s), 3.86 (2H, t, J=6.4 Hz), 7.06-7.11 (2H,m), 7.19-7.21 (1H, m), 7.95 (1H, dd, J=8.8, 2.4 Hz), 8.04 (1H, d, J=8.8 Hz), 8.33 (1H, s), 8.60-8.61 (1H, m).

### Example 357

A mixture of methyl [3-isopropyl-2-(3-{3-isopropyl-1-[5-(trifluoromethyl)-2-pyridinyl]-1H-pyrazol-4-yl}propoxy)phenyl]acetate (420 mg), 1N aqueous sodium hydroxide solution (5 ml), tetrahydrofuran (5 ml) and methanol (5 ml) was stirred at room temperature for 4 hours. 1N Hydrochloric acid (5 ml) was added, and the mixture was extracted with ethyl acetate. The ethyl acetate layer was washed with saturated brine, dried (MgSO₄) and concentrated. The obtained colorless crystals were collected by filtration to give [3-isopropyl-2-(3-{3-isopropyl-1-[5-(trifluoromethyl)-2-pyridinyl]-1H-pyrazol-4-yl}propoxy)phenyl]acetic acid (310 mg, yield 76%). The crystals were recrystallized from hexane-ethyl acetate. melting point: 108-109° C.

### Example 358

To a mixture of 3-{3-propyl-1-[5-(trifluoromethyl)-2-pyridinyl]-1H-pyrazol-4-yl}-1-propanol (410 mg), methyl (2-hydroxy-3-isopropylphenyl)acetate (280 mg), tributylphosphine (560 mg) and tetrahydrofuran (30 ml) was added 1,1′-azodicarbonyldipiperidine (700 mg) at room temperature, and the mixture was stirred overnight at 65° C. The reaction solution was concentrated. Isopropyl ether was added and the insoluble material was filtered off. The filtrate was concentrated. The residue was subjected to silica gel column chromatography, and methyl [3-isopropyl-2-(3-{3-propyl-1-[5-(trifluoromethyl)-2-pyridinyl]-1H-pyrazol-4-yl}propoxy)phenyl]acetate (430 mg, yield 66%) was obtained as a colorless oil from a fraction eluted with ethyl acetate-hexane (1:9, volume ratio).

¹H-NMR (CDCl₃) δ: 1.04 (3H, t, J=7.2 Hz), 1.21 (6H, d, J=7.2 Hz), 1.73-1.81 (2H, m), 2.11-2.17 (2H, m), 2.66 (2H, t, J=8.0 Hz), 2.71 (2H, t, J=8.0 Hz), 3.25-3.31 (1H, m), 3.68 (2H, s), 3.68 (3H, s), 3.85 (2H, t, J=6.4 Hz), 7.06-7.11 (2H, m), 7.20 (1H, dd, J=7.0, 2.4 Hz), 7.96 (1H, dd, J=8.4, 2.4 Hz), 8.02 (1H, d, J=8.4 Hz), 8.34 (1H, s), 8.61 (1H, m).

### Example 359

A mixture of methyl [3-isopropyl-2-(3-{3-propyl-1-[5-(trifluoromethyl)-2-pyridinyl]-1H-pyrazol-4-yl}propoxy)phenyl]acetate (420 mg), 1N aqueous sodium hydroxide solution (5 ml), tetrahydrofuran (5 ml) and methanol (5 ml) was stirred at room temperature for 4 hours. 1N Hydrochloric acid (5 ml) was added, and the mixture was extracted with ethyl acetate. The ethyl acetate layer was washed with saturated brine, dried (MgSO₄) and concentrated. The obtained colorless crystals were collected by filtration to give [3-isopropyl-2-(3-{3-propyl-1-[5-(trifluoromethyl)-2-pyridinyl]-1H-pyrazol-4-yl}propoxy)phenyl]acetic acid (350 mg, yield 86%). The crystals were recrystallized from hexane-ethyl acetate. melting point: 102-103° C.

### Example 360

To a mixture of 3-{3-isopropyl-1-[3-(trifluoromethyl)-2-pyridinyl]-1H-pyrazol-4-yl}-1-propanol (300 mg), methyl (2-hydroxy-3-methoxyphenyl)acetate (190 mg), tributylphosphine (410 mg) and tetrahydrofuran (20 ml) was added 1,1′-azodicarbonyldipiperidine (520 mg) at room temperature, and the mixture was stirred overnight at 65° C. The reaction solution was concentrated. Isopropyl ether was added and the insoluble material was filtered off. The filtrate was concentrated. The residue was subjected to silica gel column chromatography, and methyl [2-(3-{3-isopropyl-1-[3-(trifluoromethyl)-2-pyridinyl]-1H-pyrazol-4-yl}propoxy)-3-methoxyphenyl]acetate (440 mg, yield 94%) was obtained as a colorless oil from a fraction eluted with ethyl acetate-hexane (1:9, volume ratio).

¹H-NMR (CDCl₃) δ: 1.33 (6H, d, J=6.8 Hz), 2.05-2.12 (2H, m), 2.68-2.72 (2H, m), 3.01-3.08 (1H, m), 3.67 (3H, s), 3.69 (2H, s), 3.83 (3H, s), 4.07 (2H, t, J=6.4 Hz), 6.82-6.86 (2H, m), 6.98-7.02 (1H, m), 7.31 (1H, dd, J=8.0, 4.8 Hz), 8.01 (1H, s), 8.14 (1H, dd, J=8.0, 1.6 Hz), 8.59 (1H, dd, J=4.8, 1.6 Hz).

### Example 361

A mixture of methyl [2-(3-{3-isopropyl-1-[3-(trifluoromethyl)-2-pyridinyl]-1H-pyrazol-4-yl}propoxy)-3-methoxyphenyl]acetate (420 mg), 1N aqueous sodium hydroxide solution (6 ml), tetrahydrofuran (5 ml) and methanol (6 ml) was stirred at room temperature for 4 hours. 1N Hydrochloric acid (6 ml) was added, and the mixture was extracted with ethyl acetate. The ethyl acetate layer was washed with saturated brine, dried (MgSO₄) and concentrated. The obtained colorless crystals were collected by filtration to give [2-(3-{3-isopropyl-1-[3-(trifluoromethyl)-2-pyridinyl]-1H-pyrazol-4-yl}propoxy)-3-methoxyphenyl]acetic acid (370 mg, yield 91%) as a colorless oil.

¹H-NMR (CDCl₃) δ: 1.29 (6H, d, J=6.8 Hz), 2.15-2.22 (2H, m) 2.65 (2H, t, J=7.2 Hz), 2.96-3.03 (1H, m), 3.70 (2H, s), 3.83 (3H, s), 4.05-4.09 (2H, m), 6.82-6.86 (2H, m), 7.00 (1H, t, J=8.0 Hz), 7.36 (1H, dd, J=7.6, 4.8 Hz), 8.07 (1H, s), 8.18 (1H, dd, J=7.6, 1.6 Hz), 8.57 (1H, dd, J=4.8, 1.6 Hz).

### Example 362

To a mixture of 3-{3-isopropyl-1-[4-(trifluoromethyl)-2-pyridinyl]-1H-pyrazol-4-yl}-1-propanol (300 mg), methyl (2-hydroxy-3-methoxyphenyl)acetate (190 mg), tributylphosphine (410 mg) and tetrahydrofuran (20 ml) was added 1,1′-azodicarbonyldipiperidine (520 mg) at room temperature, and the mixture was stirred overnight at 65° C. The reaction solution was concentrated. Isopropyl ether was added and the insoluble material was filtered off. The filtrate was concentrated. The residue was subjected to silica gel column chromatography, and methyl [2-(3-{3-isopropyl-1-[4-(trifluoromethyl)-2-pyridinyl]-1H-pyrazol-4-yl}propoxy)-3-methoxyphenyl]acetate (400 mg, yield 85%) was obtained as a colorless oil from a fraction eluted with ethyl acetate-hexane (1:9, volume ratio).

¹H-NMR (CDCl₃) δ: 1.35 (6H, d, J=6.8 Hz), 2.05-2.12 (2H, m), 2.68-2.72 (2H, m), 3.03-3.10 (1H, m), 3.67 (3H, s), 3.69 (2H, s), 3.85 (3H, s), 4.07 (2H, t, J=6.4 Hz), 6.83-6.86 (2H, m), 6.99-7.03 (1H, m), 7.28 (1H, d, J=5.2 Hz), 8.16 (1H, s), 8.31 (1H, s), 8.50 (1H, d, J=5.2 Hz).

### Example 363

A mixture of methyl [2-(3-{3-isopropyl-1-[4-(trifluoromethyl)-2-pyridinyl]-1H-pyrazol-4-yl}propoxy)-3-methoxyphenyl]acetate (310 mg), 1N aqueous sodium hydroxide solution (6 ml), tetrahydrofuran (5 ml) and methanol (6 ml) was stirred at room temperature for 4 hours. 1N Hydrochloric acid (6 ml) was added, and the mixture was extracted with ethyl acetate. The ethyl acetate layer was washed with saturated brine, dried (MgSO₄) and concentrated. The obtained colorless crystals were collected by filtration to give [2-(3-{3-isopropyl-1-[4-(trifluoromethyl)-2-pyridinyl]-1H-pyrazol-4-yl}propoxy)-3-methoxyphenyl]acetic acid (260 mg, yield 86%). melting point: 127-128° C.

### Example 364

To a mixture of 3-{3-isopropyl-1-[6-(trifluoromethyl)-2-pyridinyl]-1H-pyrazol-4-yl}-1-propanol (300 mg), methyl (2-hydroxy-3-methoxyphenyl)acetate (190 mg), tributylphosphine (410 mg) and tetrahydrofuran (20 ml) was added 1,1′-azodicarbonyldipiperidine (520 mg) at room temperature, and the mixture was stirred overnight at 65° C. The reaction solution was concentrated. Isopropyl ether was added and the insoluble material was filtered off. The filtrate was concentrated. The residue was subjected to silica gel column chromatography, and methyl [2-(3-{3-isopropyl-1-[6-(trifluoromethyl)-2-pyridinyl]-1H-pyrazol-4-yl}propoxy)-3-methoxyphenyl]acetate (390 mg, yield 83%) was obtained as a colorless oil from a fraction eluted with ethyl acetate-hexane (1:9, volume ratio).

¹H-NMR (CDCl₃) δ: 1.34 (6H, d, J=6.8 Hz), 2.05-2.12 (2H, m) 2.68-2.72 (2H, m), 3.03-3.09 (1H,m), 3.68 (3H, s), 3.69 (2H, s), 3.86 (3H, s), 4.07 (2H, t, J=6.4 Hz), 6.83-6.87 (2H, m), 6.99-7.03 (1H, m), 7.44 (1H, d, J=7.6 Hz), 7.88-7.92 (1H, m), 8.13 (1H, d, J=8.4 Hz), 8.35 (1H, s).

### Example 365

A mixture of methyl [2-(3-{3-isopropyl-1-[6-(trifluoromethyl)-2-pyridinyl]-1H-pyrazol-4-yl}propoxy)-3-methoxyphenyl]acetate (360 mg), 1N aqueous sodium hydroxide solution (6 ml), tetrahydrofuran (5 ml) and methanol (6 ml) was stirred at room temperature for 4 hours. 1N Hydrochloric acid (6 ml) was added, and the mixture was extracted with ethyl acetate. The ethyl acetate layer was washed with saturated brine, dried (MgSO₄) and concentrated. The obtained colorless crystals were collected by filtration to give [2-(3-{3-isopropyl-1-[6-(trifluoromethyl)-2-pyridinyl]-1H-pyrazol-4-yl}propoxy)-3-methoxyphenyl]acetic acid (290 mg, yield 83%). melting point: 95-97° C.

### Example 366

To a mixture of 3-{3-tert-butyl-1-[6-(trifluoromethyl)pyridazin-3-yl]-1H-pyrazol-4-yl}-1-propanol (300 mg), methyl (2-hydroxy-3-methoxyphenyl)acetate (215 mg), tributylphosphine (454 μl) and tetrahydrofuran (60 ml) was added 1,1′-azodicarbonyldipiperidine (460 mg) at room temperature, and the mixture was stirred overnight. The reaction solution was concentrated. The residue was subjected to silica gel column chromatography, and a pale-yellow oily substance was obtained from a fraction eluted with ethyl acetate-hexane (1:10, volume ratio). A mixture of the obtained oily substance, 1N aqueous sodium hydroxide solution (6 ml), tetrahydrofuran (6 ml) and methanol (6 ml) was stirred at room temperature for 1 hour. 1N Hydrochloric acid (6 ml) was added, and the mixture was extracted with ethyl acetate. The ethyl acetate layer was washed with saturated brine, dried (MgSO₄) and concentrated. The residue was subjected to silica gel column chromatography. The fraction eluted with ethyl acetate-hexane (1:1, volume ratio) was concentrated and crystallized from ethyl acetate-hexane to give [2-(3-{3-tert-butyl-1-[6-(trifluoromethyl)pyridazin-3-yl]-1H-pyrazol-4-yl}propoxy)-3-methoxyphenyl]acetic acid (261 mg, yield 58%) as colorless crystals. melting point: 131-132° C.

### Example 367

To a mixture of 3-{3-tert-butyl-1-[6-(trifluoromethyl)pyridazin-3-yl]-1H-pyrazol-4-yl}-1-propanol (350 mg), methyl (2-hydroxy-3-methylphenyl)acetate (202 mg), tributylphosphine (558 μl) and tetrahydrofuran (50 ml) was added 1,1′-azodicarbonyldipiperidine (565 mg) at room temperature, and the mixture was stirred overnight. The reaction solution was concentrated. The residue was subjected to silica gel column chromatography, and a yellow oily substance was obtained from a fraction eluted with ethyl acetate-hexane (1:10, volume ratio). A mixture of the obtained oily substance, 1N aqueous sodium hydroxide solution (6 ml), tetrahydrofuran (6 ml) and methanol (6 ml) was stirred at room temperature for 1 hour. 1N Hydrochloric acid (6 ml) was added, and the mixture was extracted with ethyl acetate. The ethyl acetate layer was washed with saturated brine, dried (MgSO₄) and concentrated. The residue was subjected to silica gel column chromatography. The fraction eluted with ethyl acetate-hexane (1:1, volume ratio) was concentrated, and crystallized from ethyl acetate-hexane to give [2-(3-{3-tert-butyl-1-[6-(trifluoromethyl)pyridazin-3-yl]-1H-pyrazol-4-yl}propoxy)-3-methylphenyl]acetic acid (219 mg, yield 43%) as colorless crystals. melting point: 96-97° C.

### Example 368

To a mixture of 3-[1-(5-bromo-2-pyridinyl)-3-tert-butyl-1H-pyrazol-4-yl]-1-propanol (200 mg), methyl (2-hydroxy-3-methoxyphenyl)acetate (120 mg), tributylphosphine (240 mg) and tetrahydrofuran (40 ml), was added 1,1′-azodicarbonyldipiperidine (300 mg) at room temperature, and the mixture was stirred for 3 hours. The reaction solution was oncentrated. The precipitated crystals were removed by filtration with diethyl ether and the filtrate was concentrated. The residue was subjected to silica gel column chromatography, and an oily substance (244 mg) was obtained from a fraction eluted with ethyl acetate-hexane (1:5, volume ratio). A mixture of the obtained oily substance, 1N aqueous sodium hydroxide solution (2 ml), tetrahydrofuran (3 ml) and methanol (3 ml) was stirred at 50° C.-60° C. for 1 hour. The reaction mixture was poured into water and the mixture was acidified with 2N hydrochloric acid. The mixture was extracted with ethyl acetate. The ethyl acetate layer was washed with saturated brine, dried (MgSO₄) and concentrated. The residue was subjected to silica gel column chromatography, and (2-{3-[1-(5-bromo-2-pyridinyl)-3-tert-butyl-1H-pyrazol-4-yl]propoxy}-3-methoxyphenyl)acetic acid (210 mg, yield 71%) was obtained as a colorless oil from a fraction eluted with ethyl acetate-hexane (1:2, volume ratio).

¹H-NMR (CDCl₃) δ: 1.37 (9H, s), 2.1-2.35 (2H, m), 2.78 (2H, d, J=7.2 Hz), 3.73 (2H, s), 3.84 (3H, s), 4.12 (2H, t, J=7.2 Hz), 6.8-7.05 (3H, m), 7.86 (2H, d, J=1.4 Hz), 8.33 (1H, br s), 8.36 (1H, t, J=1.4 Hz).

### Example 369

To a mixture of 3-[1-(5-bromo-2-pyridinyl)-3-tert-butyl-1H-pyrazol-4-yl]-1-propanol (250 mg), methyl (2-hydroxy-3-methylphenyl)acetate (200 mg), tributylphosphine (450 mg) and tetrahydrofuran (30 ml) was added 1,1′-azodicarbonyldipiperidine (560 mg) at room temperature, and the mixture was stirred at 100° C. for 6 hours. The reaction solution was concentrated. The precipitated crystals were filtrated with diethyl ether and the filtrate was concentrated. The residue was subjected to silica gel column chromatography, and an oily substance (250 mg) was obtained from a fraction eluted with ethyl acetate-hexane (1:5, volume ratio). A mixture of the obtained oily substance, 1N aqueous sodium hydroxide solution (2 ml), tetrahydrofuran (2 ml) and methanol (2 ml) was stirred at 50° C.-60° C. for 1 hour. The reaction mixture was poured into water and the mixture was acidified with 2N hydrochloric acid. The mixture was extracted with ethyl acetate. The ethyl acetate layer was washed with saturated brine, dried (MgSO₄) and concentrated. The residue was subjected to silica gel column chromatography, and (2-{3-[1-(5-bromo-2-pyridinyl)-3-tert-butyl-1H-pyrazol-4-yl]propoxy}-3-methylphenyl)acetic acid (190 mg, yield 53%) was obtained as crystals from a fraction eluted with ethyl acetate-hexane (1:2, volume ratio). The crystals were recrystallized from diethyl ether-hexane. melting point: 62-63° C.

### Example 370

To a mixture of 3-[3-tert-butyl-1-(5-chloropyridin-2-yl)-1H-pyrazol-4-yl]-1-propanol (240 mg), methyl (2-hydroxy-3-methoxyphenyl)acetate (190 mg), tributylphosphine (320 mg) and tetrahydrofuran (60 ml) was added 1,1′-azodicarbonyldipiperidine (400 mg) at room temperature, and the mixture was stirred for 15 hours. The reaction solution was concentrated. The residue was subjected to silica gel column chromatography, and a colorless oil was obtained from a fraction eluted with ethyl acetate-hexane (1:9, volume ratio). A mixture of the obtained oily substance, 1N aqueous sodium hydroxide solution (2 ml), tetrahydrofuran (2 ml) and ethanol (2 ml) was stirred at 50° C. for 30 minutes and 1N hydrochloric acid (2 ml) and water were added. The mixture was extracted with ethyl acetate. The ethyl acetate layer was washed with saturated brine, dried (MgSO₄) and concentrated. The residue was subjected to silica gel column chromatography, and (2-(3-[3-tert-butyl-1-(5-chloropyridin-2-yl)-1H-pyrazol-4-yl]propoxy)-3-methoxyphenyl)acetic acid (230 mg, yield 61%) was obtained as colorless crystals from a fraction eluted with ethyl acetate-hexane (1:1, volume ratio). The crystals were recrystallized from hexane-diethyl ether to give colorless prism crystals. melting point: 87-88° C.

### Example 371

To a mixture of 3-[3-tert-butyl-1-(5-chloropyridin-2-yl)-1H-pyrazol-4-yl]-1-propanol (240 mg), methyl (2-hydroxy-3-methylphenyl)acetate (180 mg), tributylphosphine (640 mg) and tetrahydrofuran (50 ml) was added 1,1′-azodicarbonyldipiperidine (800 mg) at room temperature, and the mixture was heated under reflux for 4 hours. The reaction solution was concentrated. The residue was subjected to silica gel column chromatography, and a colorless oil was obtained from a fraction eluted with ethyl acetate-hexane (0:100 to 5:95, volume ratio). A mixture of the obtained oily substance, 1N aqueous sodium hydroxide solution (2 ml), tetrahydrofuran (2 ml) and ethanol (2 ml) was stirred at 50° C. for 30 minutes. 1N Hydrochloric acid (2 ml) and water were added, and the mixture was extracted with ethyl acetate. The ethyl acetate layer was washed with saturated brine, dried (MgSO₄) and concentrated to give (2-{3-[3-tert-butyl-1-(5-chloropyridin-2-yl)-1H-pyrazol-4-yl]propoxy}-3-methylphenyl)acetic acid (150 mg, yield 42%) as colorless crystals. The crystals were recrystallized from hexane-diethyl ether to give colorless prism crystals. melting point: 112-113° C.

### Example 372

To a mixture of [3-methyl-1-(2-pyridinyl)-1H-pyrazol-4-yl]methanol (2.30 g), ethyl 3-(4-hydroxyphenyl)propionate (2.58 g), tributylphosphine (4.93 g) and tetrahydrofuran (100 ml) was added 1,1′-azodicarbonyldipiperidine (6.16 g) at room temperature, and the mixture was stirred overnight at room temperature. The reaction solution was concentrated. The residue was subjected to silica gel column chromatography, and a colorless oil was obtained from a fraction eluted with ethyl acetate-hexane (10:90, volume ratio). A mixture of the obtained oily substance, 1N aqueous sodium hydroxide solution (15 ml), tetrahydrofuran (15 ml) and ethanol (15 ml) was stirred at room temperature for 2 hours. 1N Hydrochloric acid (15 ml) and water were added, and the mixture was extracted with ethyl acetate. The ethyl acetate layer was washed with saturated brine, dried (MgSO₄) and concentrated to give 3-{4-[3-methyl-1-(2-pyridinyl)-1H-pyrazol-4-ylmethoxy]phenyl}propionic acid (3.05 g, yield 74%) as colorless crystals. The crystals were recrystallized from ethanol-water. melting point: 129-130° C.

### Example 373

To a mixture of [3-methyl-1-(2-pyridinyl)-1H-pyrazol-4-yl]methanol (3.14 g), methyl (4-hydroxyphenyl)acetate (3.00 g), tributylphosphine (6.71 g) and tetrahydrofuran (100 ml) was added 1,1′-azodicarbonyldipiperidine (8.36 g) at room temperature, and the mixture was stirred overnight at room temperature. The reaction solution was concentrated. The residue was subjected to silica gel column chromatography, and a colorless oil was obtained from a fraction eluted with ethyl acetate-hexane (10:90, volume ratio). A mixture of the obtained oily substance, 1N aqueous sodium hydroxide solution (20 ml), tetrahydrofuran (20 ml) and ethanol (20 ml) was stirred at room temperature for 2 hours. 1N Hydrochloric acid (20 ml) and water were added, and the mixture was extracted with ethyl acetate. The ethyl acetate layer was washed with saturated brine, dried (MgSO₄) and concentrated to give {4-[3-methyl-1-(2-pyridinyl)-1H-pyrazol-4-ylmethoxy]phenyl}acetic acid (3.15 g, yield 59%) as colorless crystals. The crystals were recrystallized from ethanol-water. melting point: 161-162° C.

### Example 374

To a mixture of 3-{3-ethoxy-1-[5-(trifluoromethyl)-2-pyridinyl]-1H-pyrazol-4-yl}-1-propanol (500 mg), ethyl 2-(3-hydroxy-4-methylphenoxy)-2-methylpropanonate (455 mg), tributylphosphine (792 μL) and tetrahydrofuran (60 ml) was added 1,1′-azodicarbonyldipiperidine (802 mg) at room temperature, and the mixture was stirred for 3 hours. The reaction solution was concentrated. The residue was subjected to silica gel column chromatography, and a colorless oil was obtained from a fraction eluted with ethyl acetate-hexane (1:9, volume ratio). A mixture of the obtained oily substance, 1N aqueous sodium hydroxide solution (25 ml), tetrahydrofuran (25 ml) and ethanol (25 ml) was stirred at room temperature for 3 hours. 1N Hydrochloric acid (25 ml) was added, and the mixture was extracted with ethyl acetate. The ethyl acetate layer was washed with saturated brine, dried (MgSO₄) and concentrated. The residue was subjected to silica gel column chromatography, and a colorless oil was obtained from a fraction eluted with ethyl acetate-hexane (3:7, volume ratio). A mixture of the obtained oily substance, 1N aqueous sodium hydroxide solution (1.20 ml), tetrahydrofuran (25 ml) and ethanol (25 ml) was stirred at room temperature for 1 hour and concentrated. To a mixture of the residue and water (50 ml) was added calcium chloride (127 mg) dissolved in a small amount of water and the mixture was stirred at room temperature for 3 hours. The resulting white precipitates were collected by filtration to give calcium 2-[3-(3-{3-ethoxy-1-[5-(trifluoromethyl)-2-pyridinyl]-1H-pyrazol-4-yl}propoxy)-4-methylphenoxy]-2-methylpropanoate (521 mg, yield 62%) as amorphous.

¹H-NMR (DMSO-d₆) δ: 1.34 (3H, t, J=7.0 Hz), 1.38 (6H, s), 1.92-2.09 (2H, m), 2.04 (3H, s), 2.46-2.61 (2H, m), 3.85-3.97 (2H, m), 4.31 (2H, q, J=7.0 Hz), 6.31-6.39 (1H, m), 6.41-6.47 (1H, m), 6.85 (1H, d, J=8.0 Hz), 7.81 (1H, d, J=8.8 Hz), 8.22-8.30 (1H, m), 8.34 (1H, s), 8.70-8.76 (1H, m).

### Example 375

To a mixture of 3-{3-isopropyl-1-[5-(trifluoromethyl)-2-pyridinyl]-1H-pyrazol-4-yl}-1-propanol (500 mg), ethyl 2-(3-hydroxy-4-methylphenoxy)-2-methylpropanoate (458 mg), tributylphosphine (797 μL) and tetrahydrofuran (50 ml) was added 1,1′-azodicarbonyldipiperidine (807 mg) at room temperature, and the mixture was stirred overnight. The reaction solution was concentrated. The residue was subjected to silica gel column chromatography, and a colorless oil was obtained from a fraction eluted with ethyl acetate-hexane (1:9, volume ratio). A mixture of the obtained oily substance, 1N aqueous sodium hydroxide solution (25 ml), tetrahydrofuran (25 ml) and ethanol (25 ml) was stirred at room temperature for 3 hours. 1N Hydrochloric acid (25 ml) was added, and the mixture was extracted with ethyl acetate. The ethyl acetate layer was washed with saturated brine, dried (MgSO₄) and concentrated. A mixture of the residue, 1N aqueous sodium hydroxide solution (1.50 ml), tetrahydrofuran (25 ml) and ethanol (25 ml) was stirred at room temperature for 1 hour and concentrated. To a mixture of the residue and water (50 ml) was added calcium chloride (158 mg) dissolved in a small amount of water and the mixture was stirred at room temperature for 1 hour. The resulting white precipitates were collected by filtration to give calcium 2-[3-(3-{3-isopropyl-1-[5-(trifluoromethyl)-2-pyridinyl]-1H-pyrazol-4-yl}propoxy)-4-methylphenoxy]-2-methylpropanoate (647 mg, yield 77%) as amorphous.

¹H-NMR (DMSO-d₆) δ: 1.26 (6H, d, J=6.8 Hz), 1.39 (6H, s), 1.92-2.12 (2H, m), 2.05 (3H, s), 2.56-2.72 (2H, m), 2.92-3.10 (1H, m), 3.87-4.02 (2H, m), 6.32-6.41 (1H, m), 6.43-6.49 (1H, m), 6.85 (1H, d, J=8.4 Hz), 7.98 (1H, d, J=8.8 Hz), 8.29 (1H, dd, J=2.2, 8.8 Hz), 8.39 (1H, s), 8.74-8.82 (1H, m).

### Example 376

To a mixture of 3-{3-isopropyl-1-[5-(trifluoromethyl)-2-pyridinyl]-1H-pyrazol-4-yl}-1-propanol (500 mg), ethyl 2-(3-hydroxy-4-methoxyphenoxy)-2-methylpropanoate (488 mg), tributylphosphine (797 μL) and tetrahydrofuran (50 ml) was added 1,1′-azodicarbonyldipiperidine (807 mg) at room temperature, and the mixture was stirred overnight. The reaction solution was concentrated. The residue was subjected to silica gel column chromatography, and a colorless oil was obtained from a fraction eluted with ethyl acetate-hexane (1:9, volume ratio). A mixture of the obtained oily substance, 1N aqueous sodium hydroxide solution (25 ml), tetrahydrofuran (25 ml) and ethanol (25 ml) was stirred at room temperature for 5 hours. 1N Hydrochloric acid (25 ml) was added, and the mixture was extracted with ethyl acetate. The ethyl acetate layer was washed with saturated brine, dried (MgSO₄) and concentrated. The obtained pale-yellow solid was recrystallized from diisopropyl ether-hexane to give 2-[3-(3-{3-isopropyl-1-[5-(trifluoromethyl)-2-pyridinyl]-1H-pyrazol-4-yl}propoxy)-4-methoxyphenoxy]-2-methylpropanoic acid (610 mg, yield 73%) as colorless crystals. melting point: 106-107° C.

### Example 377

To a mixture of 3-{3-isopropyl-1-[5-(trifluoromethyl)-2-pyridinyl]-1H-pyrazol-4-yl}-1-propanol (500 mg), ethyl 2-(4-ethyl-3-hydroxyphenoxy)-2-methylpropanoate (484 mg), tributylphosphine (797 μL) and tetrahydrofuran (50 ml) was added 1,1′-azodicarbonyldipiperidine (807 mg) at room temperature, and the mixture was stirred for 1.5 days. The reaction solution was concentrated. The residue was subjected to silica gel column chromatography, and a colorless oil was obtained from a fraction eluted with ethyl acetate-hexane (3:37, volume ratio). A mixture of the obtained oily substance, 1N aqueous sodium hydroxide solution (25 ml), tetrahydrofuran (25 ml) and ethanol (25 ml) was stirred at room temperature for 5 hours. 1N Hydrochloric acid (25 ml) was added, and the mixture was extracted with ethyl acetate. The ethyl acetate layer was washed with saturated brine, dried (MgSO₄) and concentrated. The obtained white solid was recrystallized from diisopropyl ether-hexane to give 2-[4-ethyl-3-(3-{3-isopropyl-1-[5-(trifluoromethyl)-2-pyridinyl]-1H-pyrazol-4-yl}propoxy)phenoxy]-2-methylpropanoic acid (565 mg, yield 68%) as colorless crystals. melting point: 115-116° C.

### Example 378

A mixture of 2-(3-{3-isopropyl-1-[5-(trifluoromethyl)pyridin-2-yl]-1H-pyrazol-4-yl}propoxy)-3-methoxybenzaldehyde (0.4 g), tetrahydrofuran (4 ml), methyl methylthiomethyl sulfoxide (0.14 ml) and sodium hydroxide (0.04 g) was reacted for about 5 hours under reflux. The mixture was cooled to room temperature and water was added. The mixture was extracted with ethyl acetate. The aqueous layer was further extracted with ethyl acetate. The organic layers were combined and washed twice with water. The organic layer was concentrated and a mixture of the residue, ethanol (8 ml) and conc. hydrochloric acid (0.74 ml) was stirred at 80° C. for about 14 hours. The mixture was cooled to room temperature and 1N aqueous sodium hydroxide solution (10.7 ml) and ethanol (20 ml) were added. The mixture was stirred at about 70° C. for 1 hour. The mixture was cooled to room temperature and water (10 ml) and toluene (10 ml) were added. 1N Aqueous sodium hydroxide solution (10 ml) was added to the organic layer and partitioned. The aqueous layers were combined and conc. hydrochloric acid was dropwise added. The mixture was adjusted to pH 2.0 and extracted with ethyl acetate. The organic layer was washed twice with water, and heptane (2 ml) was added. The mixture was stirred at room temperature for about 0.5 hour. The precipitated crystals were collected by filtration and washed with heptane to give [2-(3-{3-isopropyl-1-(5-trifluoromethylpyridin-2-yl)-1H-pyrazol-4-yl}propoxy)-3-methoxyphenyl]acetic acid (0.30 g, yield 70.3%) as pale-yellow white crystals.

¹H-NMR (CDCl₃) δ: 1.32 (6H, d, J=6.9 Hz), 2.1-2.2 (2H, m), 2.6-2.7 (2H, m), 3.0-3.1 (1H, m), 3.71 (2H, s), 3.84 (3H, s), 4.0-4.2 (2H, m), 6.8-7.1 (3H, m), 7.90 (1H, dd, J=8.7, 2.2 Hz), 8.04 (1H, d, J=8.7 Hz), 8.36 (1H, s), 8.5-8.6 (1H, m).

### Example 379

A mixture of 2-(3-{3-(1-ethylpropyl)-1-[5-(trifluoromethyl)pyridin-2-yl]-1H-pyrazol-4-yl}propoxy)-3-methoxybenzaldehyde (585 mg), tetrahydrofuran (5.85 ml), methyl methylthiomethyl sulfoxide (0.193 ml) and sodium hydroxide (0.049 g) was stirred for about 5 hours under reflux. The mixture was cooled to room temperature and water was added. The mixture was extracted with ethyl acetate and the aqueous layer was further extracted with ethyl acetate. The organic layers were combined, washed with water, and the organic layer was concentrated. A mixture of the residue, ethanol (10.5 ml) and 4N hydrochloric acid-ethyl acetate (3.05 ml) was stirred at about 80° C. for about 1 hour. The mixture was cooled to room temperature. Saturated aqueous sodium hydrogen carbonate was added, and the mixture was extracted with ethyl acetate. The organic layer was washed with water, dried over anhydrous magnesium sulfate and concentrated under reduced pressure to give ethyl [2-(3-{3-(1-ethylpropyl)-1-[5-(trifluoromethyl)pyridin-2-yl]-1H-pyrazol-4-yl}propoxy)-3-methoxyphenyl]acetate (537 mg, yield 82%) as an oily substance.

¹H-NMR (CDCl₃) δ: 0.8-0.9 (6H, m), 1.2-1.3 (3H, m), 1.6-1.8 (4H, m), 2.0-2.1 (2H, m), 2.6-2.7 (3H, m), 3.68 (2H, s), 3.85 (3H, s), 4.0-4.2 (2H, m), 6.8-6.9 (2H, m), 7.0-7.1 (1H, m), 7.94 (1H, dd, J=8.7, 2.2 Hz), 8.04 (1H, d, J=8.7 Hz), 8.33 (1H, s), 8.6-8.7 (1H, m).

### Preparation Example 1

### Production of Capsules

| | |
|---|---|
| 1) Compound of Example 1 | 30 mg |
| 2) Finely divided cellulose | 10 mg |
| 3) Lactose | 19 mg |
| 4) Magnesium stearate | 1 mg |
| Total | 60 mg |

1), 2), 3) and 4) are admixed and filled into a gelatin capsule.

### Preparation Example 2

### Production of Tablets

| | |
|---|---|
| 1) Compound of Example 1 | 30 g |
| 2) Lactose | 50 g |
| 3) Corn starch | 15 g |
| 4) Carboxymethylcellulose calcium | 44 g |
| 5) Magnesium stearate | 1 g |
| 1000 tablets | 140 g |

The whole amounts of 1), 2) and 3) and 30 g of 4) are kneaded together with water and the mixture, after vacuum drying, is granulated. The granular mixture is admixed with 14 g of 4) and 1 g of 5) and the resulting mixture is tableted using a tableting machine, to give 1000 tablets each containing 30 mg of compound of Example 1.

### INDUSTRIAL APPLICABILITY

The compound of the present invention is superior in a hypoglycemic action, a hypolipidemic action, a hypoinsulinemic action, insulin resistance-improving action, insulin sensitivity enhancing action and retinoid-related receptor function regulating activity, and can be used as an agent for the prophylaxis or treatment of diabetes (e.g., type 1 diabetes, type 2 diabetes, gestational diabetes); an agent for the prophylaxis or treatment of hyperlipidemia (e.g., hypertriglyceridemia, hypercholesterolemia, hypo-high-density-lipoproteinemia, postprandial hyperlipemia); an agent for improving insulin resistance; an agent for enhancing insulin sensitivity; an agent for the prophylaxis or treatment of impaired glucose tolerance [IGT]; and an agent for preventing progress from impaired glucose tolerance to diabetes.

This application is based on patent application Nos. 2002-151405, 2002-287161 and 2003-16748 filed in Japan, the contents of which are hereby incorporated by reference.

1. A compound represented by the formula 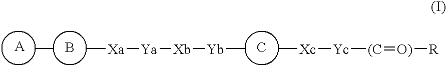
wherein
ring A is a ring optionally having 1 to 3 substituents;
ring B is a 1,2-azole ring optionally further having 1 to 3 substituents;
Xa, Xb and Xc
are the same or different and each is a bond, —O—, —S—, —SO—, —SO₂—, —CO—, —CS—, —CR¹(OR²)—, —NR³—, —CONR³— or —NR³CO— (R¹ is a hydrogen atom or an optionally substituted hydrocarbon group, R² is a hydrogen atom or a hydroxy-protecting group, and R³ is a hydrogen atom, an optionally substituted hydrocarbon group or an amino-protecting group);
Ya is a divalent aliphatic hydrocarbon residue having 1 to 20 carbon atoms;
Yb and Yc
are the same or different and each is a bond or a divalent aliphatic hydrocarbon residue having 1 to 20 carbon atoms;
ring C is a monocyclic aromatic ring optionally further having 1 to 3 substituents; and
R represents —OR⁴ (R⁴ is a hydrogen atom or an optionally substituted hydrocarbon group) or —NR⁵R⁶ (R⁵ and R⁶ are the same or different and each is a hydrogen atom, an optionally substituted hydrocarbon group or an optionally substituted heterocyclic group, or R⁵ and R⁶ form, together with the adjacent nitrogen atom, an optionally substituted heterocyclic ring),
provided that,
(1) when the 1,2-azole ring represented by ring B is pyrazole, ring C is not thiadiazole or oxadiazole;
(2) when the 1,2-azole ring represented by ring B is isoxazole, ring C is not an optionally substituted pyridone; and
(3) when the 1,2-azole ring represented by ring B is pyrazole and Xa and Xb are each a bond, ring C is not a benzene ring,
or a salt thereof.

2. The compound of claim 1, wherein the ring represented by ring A is an aromatic ring.

3. The compound of claim 2, wherein the aromatic ring is a benzene ring, a pyridine ring or a pyridazine ring.

4. The compound of claim 1, wherein the 1,2-azole ring represented by ring B is pyrazole.

5. The compound of claim 1, wherein the substituent that ring B is optionally further having is a hydrocarbon group.

6. The compound of claim 1, wherein the substituent that ring B is optionally further having is an alkoxy group.

7. The compound of claim 1, wherein Ya is C₁₋₆ alkylene or C₂₋₆ alkenylene.

8. The compound of claim 1, wherein Xb is —O—, —S—, —SO—, —SO₂—, —CO—, —CS—, —CR¹(OR²)—, —NR³—, —CONR³— or —NR³CO— (R¹ is a hydrogen atom or an optionally substituted hydrocarbon group, R² is a hydrogen atom or a hydroxy-protecting group, and R³ is a hydrogen atom, an optionally substituted hydrocarbon group or an amino-protecting group).

9. The compound of claim 1, wherein the monocyclic aromatic ring represented by ring C is a benzene ring.

10. The compound of claim 1, wherein the monocyclic aromatic ring represented by ring C is pyrazole.

11. The compound of claim 1, wherein R represents —OR⁴ (R⁴ is a hydrogen atom or an optionally substituted hydrocarbon group).

12. The compound of claim 1, wherein Xa is a bond.

13. The compound of claim 1, wherein Xb is —O—.

14. The compound of claim 1, wherein Yb is a bond.

15. The compound of claim 1, wherein Xc is a bond or —O—.

16. The compound of claim 1, wherein Yc is C₁₋₆ alkylene or C₂₋₆ alkenylene.

17. The compound of claim 1, which is 3-[1-phenyl-3-(4-{3-[4-(trifluoromethyl)phenyl]-5-isoxazolyl}butoxy)-1H-pyrazol-5-yl]propionic acid;
2-[3-(3-{3-ethoxy-1-[5-(trifluoromethyl)-2-pyridyl]-1H-pyrazol-4-yl}propoxy)phenoxy]-2-methylpropionic acid;
3-[2-ethoxy-4-(3-{3-ethoxy-1-[5-(trifluoromethyl)-2-pyridyl]-1H-pyrazol-4-yl}propoxy)phenyl]propionic acid;
3-[3-(3-{3-ethoxy-1-[5-(trifluoromethyl)-2-pyridyl]-1H-pyrazol-4-yl} propoxy)-1-phenyl-1H-pyrazol-5-yl]propionic acid;
[1-phenyl-3-(4-{3-propyl-1-[5-(trifluoromethyl)-2-pyridinyl]-1H-pyrazol-4-yl}butoxy)-1H-pyrazol-4-yl]acetic acid; [2-(3-{3-isopropyl-1-[5-(trifluoromethyl)-2-pyridyl]-1H-pyrazol-4-yl}propoxy)-3-methoxyphenyl]acetic acid;
[2-(3-{3-(1-ethylpropyl)-1-[5-(trifluoromethyl)-2-pyridyl]-1H-pyrazol-4-yl}propoxy)-3-methoxyphenyl]acetic acid;
(2-{3-[1-(5-chloro-2-pyridyl)-3-(1-ethylpropyl)-1H-pyrazol-4-yl]propoxy}-3-methoxyphenyl)acetic acid;
[3-ethyl-2-(3-{3-isopropyl-1-[6-(trifluoromethyl)pyridazin-3-yl]-1H-pyrazol-4-yl}propoxy)phenyl]acetic acid;
[2-(3-{3-isopropyl-1-[6-(trifluoromethyl)pyridazin-3-yl]-1H-pyrazol-4-yl}propoxy)-3-methoxyphenyl]acetic acid;
[3-(3-{3-isopropyl-1-[5-(trifluoromethyl)-2-pyridinyl]-1H-pyrazol-4-yl}propoxy)-1-methyl-1H-pyrazol-4-yl]acetic acid;
[1-ethyl-5-(3-{3-isopropyl-1-[5-(trifluoromethyl)-2-pyridinyl]-1H-pyrazol-4-yl}propoxy)-1H-pyrazol-4-yl]acetic acid;
[1-ethyl-5-(3-{3-propyl-1-[5-(trifluoromethyl)-2-pyridinyl]-1H-pyrazol-4-yl}propoxy)-1H-pyrazol-4-yl]acetic acid;
(2-{3-[1-(5-bromo-2-pyridinyl)-3-(1-ethylpropyl)-1H-pyrazol-4-yl]propoxy}-3-methoxyphenyl)acetic acid; or
[2-(3-{3-tert-butyl-1-[6-(trifluoromethyl)pyridazin-3-yl]-1H-pyrazol-4-yl}propoxy)-3-methylphenyl]acetic acid.

18. A prodrug of the compound of claim 1 or a salt thereof.

19. A pharmaceutical composition comprising the compound of claim 1 or a salt thereof or a prodrug thereof, and a pharmaceutically acceptable carrier excipient or diluent.

20. A pharmaceutical composition for the prophylaxis or treatment of diabetes, which comprises a compound represented by the formula 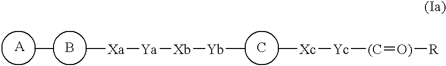
wherein
ring A is a ring optionally having 1 to 3 substituents;
ring B is a 1,2-azole ring optionally further having 1 to 3 substituents;
Xa, Xb and Xc
are the same or different and each is a bond, —O—, —S—, —SO—, —SO₂—, —CO—, —CS—, —CR¹(OR²)—, —NR³—, —CONR³— or —NR³CO— (R¹ is a hydrogen atom or an optionally substituted hydrocarbon group, R² is a hydrogen atom or a hydroxy-protecting group, and R³ is a hydrogen atom, an optionally substituted hydrocarbon group or an amino-protecting group);
Ya is a divalent aliphatic hydrocarbon residue having 1 to 20 carbon atoms;
Yb and Yc
are the same or different and each is a bond or a divalent aliphatic hydrocarbon residue having 1 to 20 carbon atoms;
ring C is a monocyclic aromatic ring optionally further having 1 to 3 substituents; and
R represents —OR⁴ (R⁴ is a hydrogen atom or an optionally substituted hydrocarbon group) or —NR⁵R⁶ (R⁵ and R⁶ are the same or different and each is a hydrogen atom, an optionally substituted hydrocarbon group or an optionally substituted heterocyclic group, or R⁵ and R⁶ form, together with the adjacent nitrogen atom, an optionally substituted heterocyclic ring),
or a salt thereof or a prodrug thereof, and a pharmaceutically acceptable carrier excipient or diluent.

21. A pharmaceutical composition for the prophylaxis or treatment of hyperlipidemia, which comprises a compound represented by the formula 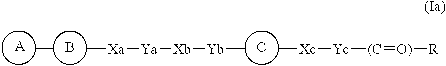
wherein
ring A is a ring optionally having 1 to 3 substituents;
ring B is a 1,2-azole ring optionally further having 1 to 3 substituents;
Xa, Xb and Xc
are the same or different and each is a bond, —O—, —S—, —SO—, —SO₂—, —CO—, —CS—, —CR¹(OR²)—, —NR³—, —CONR³— or —NR³CO— (R₁ is a hydrogen atom or an optionally substituted hydrocarbon group, R² is a hydrogen atom or a hydroxy-protecting group, and R³ is a hydrogen atom, an optionally substituted hydrocarbon group or an amino-protecting group);
Ya is a divalent aliphatic hydrocarbon residue having 1 to 20 carbon atoms;
Yb and Yc
are the same or different and each is a bond or a divalent aliphatic hydrocarbon residue having 1 to 20 carbon atoms;
ring C is a monocyclic aromatic ring optionally further having 1 to 3 substituents; and
R represents —OR⁴ (R⁴ is a hydrogen atom or an optionally substituted hydrocarbon group) or —NR⁵R⁶ (R⁵ and R⁶ are the same or different and each is a hydrogen atom, an optionally substituted hydrocarbon group or an optionally substituted heterocyclic group, or R⁵ and R⁶ form, together with the adjacent nitrogen atom, an optionally substituted heterocyclic ring),
or a salt thereof or a prodrug thereof, and a pharmaceutically acceptable carrier, excipient or diluent.

22. A pharmaceutical composition for the prophylaxis or treatment of arteriosclerosis, which comprises a compound represented by the formula 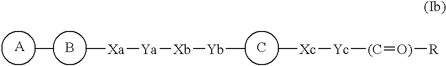
wherein
ring A is a ring optionally having 1 to 3 substituents;
ring B is a 1,2-azole ring optionally further having 1 to 3 substituents;
Xa, Xb and Xc
are the same or different and each is a bond, —O—, —S—, —SO—, —SO₂—, —CO—, —CS—, —CR¹(OR²)—, —NR³—, —CONR³— or —NR³CO— (R¹ is a hydrogen atom or an optionally substituted hydrocarbon group, R² is a hydrogen atom or a hydroxy-protecting group, and R³ is a hydrogen atom, an optionally substituted hydrocarbon group or an amino-protecting group);
Ya is a divalent aliphatic hydrocarbon residue having 1 to 20 carbon atoms;
Yb and Yc
are the same or different and each is a bond or a divalent aliphatic hydrocarbon residue having 1 to 20 carbon atoms;
ring C is a monocyclic aromatic ring optionally further having 1 to 3 substituents; and
R represents —OR⁴ (R⁴ is a hydrogen atom or an optionally substituted hydrocarbon group) or —NR⁵R⁶ (R⁵ and R⁶ are the same or different and each is a hydrogen atom, an optionally substituted hydrocarbon group or an optionally substituted heterocyclic group, or R⁵ and R⁶ form, together with the adjacent nitrogen atom, an optionally substituted heterocyclic ring),
provided that, when the 1,2-azole ring represented by ring B is isoxazole, ring C is not an optionally substituted pyridone,
or a salt thereof or a prodrug thereof, and a pharmaceutically acceptable carrier, excipient or diluent.

23. A pharmaceutical composition for the prophylaxis or treatment of impaired glucose tolerance, which comprises a compound represented by the formula 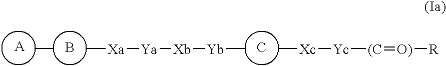
wherein
ring A is a ring optionally having 1 to 3 substituents;
ring B is a 1,2-azole ring optionally further having 1 to 3 substituents;
Xa, Xb and Xc
are the same or different and each is a bond, —O—, —S—, —SO—, —SO₂—, —CO—, —CS—, —CR¹(OR²)—, —NR³—, —CONR³— or —NR³CO— (R¹ is a hydrogen atom or an optionally substituted hydrocarbon group, R² is a hydrogen atom or a hydroxy-protecting group, and R³ is a hydrogen atom, an optionally substituted hydrocarbon group or an amino-protecting group);
Ya is a divalent aliphatic hydrocarbon residue having 1 to 20 carbon atoms;
Yb and Yc
are the same or different and each is a bond or a divalent aliphatic hydrocarbon residue having 1 to 20 carbon atoms;
ring C is a monocyclic aromatic ring optionally further having 1 to 3 substituents; and
R represents —OR⁴ (R⁴ is a hydrogen atom or an optionally substituted hydrocarbon group) or —NR⁵R⁶ (R⁵ and R⁶ are the same or different and each is a hydrogen atom, an optionally substituted hydrocarbon group or an optionally substituted heterocyclic group, or R⁵ and R⁶ form, together with the adjacent nitrogen atom, an optionally substituted heterocyclic ring),
or a salt thereof or a prodrug thereof, and a pharmaceutically acceptable carrier, excipient or diluent.

24. A pharmaceutical composition which is a retinoid-related receptor function regulating agent, which comprises a compound represented by the formula 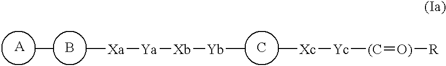
wherein
ring A is a ring optionally having 1 to 3 substituents;
ring B is a 1,2-azole ring optionally further having 1 to 3 substituents;
Xa, Xb and Xc
are the same or different and each is a bond, —O—, —S—, —SO—, —SO₂—, —CO—, —CS—, —CR¹(OR²)—, —NR³—, —CONR³— or —NR³CO— (R¹ is a hydrogen atom or an optionally substituted hydrocarbon group, R² is a hydrogen atom or a hydroxy-protecting group, and R³ is a hydrogen atom, an optionally substituted hydrocarbon group or an amino-protecting group);
Ya is a divalent aliphatic hydrocarbon residue having 1 to 20 carbon atoms;
Yb and Yc
are the same or different and each is a bond or a divalent aliphatic hydrocarbon residue having 1 to 20 carbon atoms;
ring C is a monocyclic aromatic ring optionally further having 1 to 3 substituents; and
R represents —OR⁴ (R⁴ is a hydrogen atom or an optionally substituted hydrocarbon group) or —NR⁵R⁶ (R⁵ and R⁶ are the same or different and each is a hydrogen atom, an optionally substituted hydrocarbon group or an optionally substituted heterocyclic group, or R⁵ and R⁶ form, together with the adjacent nitrogen atom, an optionally substituted heterocyclic ring),
or a salt thereof or a prodrug thereof, and a pharmaceutically acceptable carrier, excipient or diluent.

25. The agent of claim 24, which is a peroxisome proliferator-activated receptor ligand.

26. The agent of claim 24, which is a retinoid X receptor ligand.

27. A pharmaceutical composition which is an insulin resistance improving agent, which comprises a compound represented by the formula 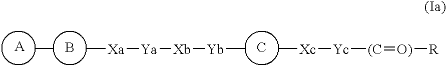
wherein
ring A is a ring optionally having 1 to 3 substituents;
ring B is a 1,2-azole ring optionally further having 1 to 3 substituents;
Xa, Xb and Xc
are the same or different and each is a bond, —O—, —S—, —SO—, —SO₂—, —CO—, —CS—, —CR¹(OR²)—, —NR³—, —CONR³— or —NR³CO— (R¹ is a hydrogen atom or an optionally substituted hydrocarbon group, R² is a hydrogen atom or a hydroxy-protecting group, and R³ is a hydrogen atom, an optionally substituted hydrocarbon group or an amino-protecting group);
Ya is a divalent aliphatic hydrocarbon residue having 1 to 20 carbon atoms;
Yb and Yc
are the same or different and each is a bond or a divalent aliphatic hydrocarbon residue having 1 to 20 carbon atoms;
ring C is a monocyclic aromatic ring optionally further having 1 to 3 substituents; and
R represents —OR⁴ (R⁴ is a hydrogen atom or an optionally substituted hydrocarbon group) or —NR⁵R⁶ (R⁵ and R⁶ are the same or different and each is a hydrogen atom, an optionally substituted hydrocarbon group or an optionally substituted heterocyclic group, or R⁵ and R⁶ form, together with the adjacent nitrogen atom, an optionally substituted heterocyclic ring),
or a salt thereof or a prodrug thereof, and a pharmaceutically acceptable carrier, excipient or diluent.

28. A method for the prophylaxis or treatment of diabetes in a mammal in need thereof, which comprises administering to the mammal a compound represented by the formula 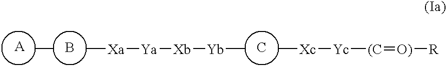
wherein
ring A is a ring optionally having 1 to 3 substituents;
ring B is a 1,2-azole ring optionally further having 1 to 3 substituents;
Xa, Xb and Xc
are the same or different and each is a bond, —O—, —S—, —SO—, —SO₂—, —CO—, —CS—, —CR¹(OR²)—, —NR³—, —CONR³— or —NR³CO— (R¹ is a hydrogen atom or an optionally substituted hydrocarbon group, R² is a hydrogen atom or a hydroxy-protecting group, and R³ is a hydrogen atom, an optionally substituted hydrocarbon group or an amino-protecting group);
Ya is a divalent aliphatic hydrocarbon residue having 1 to 20 carbon atoms;
Yb and Yc
are the same or different and each is a bond or a divalent aliphatic hydrocarbon residue having 1 to 20 carbon atoms;
ring C is a monocyclic aromatic ring optionally further having 1 to 3 substituents; and
R represents —OR⁴ (R⁴ is a hydrogen atom or an optionally substituted hydrocarbon group) or —NR⁵R⁶ (R⁵ and R⁶ are the same or different and each is a hydrogen atom, an optionally substituted hydrocarbon group or an optionally substituted heterocyclic group, or R⁵ and R⁶ form, together with the adjacent nitrogen atom, an optionally substituted heterocyclic ring),
or a salt thereof or a prodrug thereof.

29. A method for making a pharmaceutical composition with hypoglycemic or hypolipidemic activity, said method comprising combining a compound represented by the formula 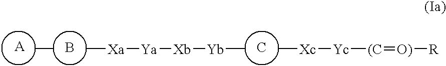
wherein
ring A is a ring optionally having 1 to 3 substituents;
ring B is a 1,2-azole ring optionally further having 1 to 3 substituents;
Xa, Xb and Xc
are the same or different and each is a bond, —O—, —S—, —SO—, —SO₂—, —CO—, —CS—, —CR¹(OR²)—, —NR³—, —CONR³— or —NR³CO— (R¹ is a hydrogen atom or an optionally substituted hydrocarbon group, R² is a hydrogen atom or a hydroxy-protecting group, and R³ is a hydrogen atom, an optionally substituted hydrocarbon group or an amino-protecting group);
Ya is a divalent aliphatic hydrocarbon residue having 1 to 20 carbon atoms;
Yb and Yc
are the same or different and each is a bond or a divalent aliphatic hydrocarbon residue having 1 to 20 carbon atoms;
ring C is a monocyclic aromatic ring optionally further having 1 to 3 substituents; and
R represents —OR⁴ (R⁴ is a hydrogen atom or an optionally substituted hydrocarbon group) or —NR⁵R⁶ (R⁵ and R⁶ are the same or different and each is a hydrogen atom, an optionally substituted hydrocarbon group or an optionally substituted heterocyclic group, or R⁵ and R⁶ form, together with the adjacent nitrogen atom, an optionally substituted heterocyclic ring),
or a salt thereof or a prodrug thereof, for the production of an agent for the prophylaxis or treatment of diabetes.

30. A pharmaceutical composition which is a GPR40 receptor function modulator comprising a compound represented by the formula 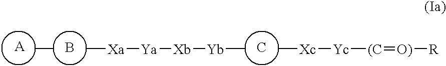
wherein
ring A is a ring optionally having 1 to 3 substituents;
ring B is 1,2-azole ring optionally further having 1 to 3 substituents;
Xa, Xb and Xc
are the same or different and each is a bond, —O—, —S—, —SO—, —SO₂—, —CO—, —CS—, —CR¹(OR²)—, —NR³—, —CONR³— or —NR³CO— (R¹ is a hydrogen atom or an optionally substituted hydrocarbon group, R² is a hydrogen atom or hydroxy-protecting group, and R³ is a hydrogen atom, an optionally substituted hydrocarbon group or an amino-protecting group);
Ya is a divalent aliphatic hydrocarbon residue having 1 to 20 carbon atoms;
Yb and Yc
are the same or different and each is a bond or a divalent aliphatic hydrocarbon residue having 1 to 20 carbon atoms;
ring C is a monocyclic aromatic ring optionally further having 1 to 3 substituents; and
R represents —OR⁴ (R⁴ is a hydrogen atom or an optionally substituted hydrocarbon group) or —NR⁵R⁶ (R⁵ and R⁶ are the same or different and each is a hydrogen atom, an optionally substituted hydrocarbon group or an optionally substituted heterocyclic group, or R⁵ and R⁶ form, together with the adjacent nitrogen atom, an optionally substituted heterocyclic ring),
or a salt thereof or a prodrug thereof, and a pharmaceutically acceptable carrier, excipient or diluent.

31. A method of producing a compound represented by the formula 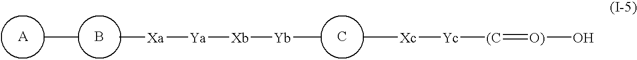
wherein the symbols in the formula are as defined in claim 1, or a salt thereof, which comprises subjecting a compound represented by the formula 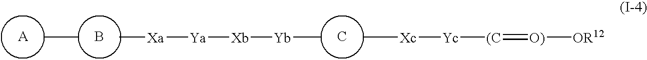
wherein R¹² is an optionally substituted hydrocarbon group and other symbols are as defined above, or a salt thereof to a hydrolysis reaction.

32. A method of producing a compound represented by the formula 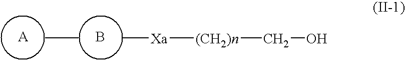
wherein n is an integer of 0 to 5 and other symbols are as defined in claim 1, or a salt thereof, which comprises subjecting a compound represented by the formula 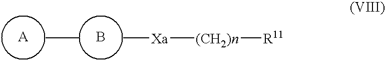
wherein R¹¹ is CHO or COOR¹³ (R¹³ is an alkyl group having 1-6 carbon atoms), and other symbols are as defined above, or a salt thereof to a reduction reaction.

33. A compound represented by the formula 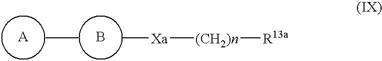
wherein n is an integer of 0 to 5, R^{13a} is CH₂OH, CHO or COOR¹⁴ (R¹⁴ is an alkyl group having 1-6 carbon atoms), and other symbols are as defined in claim 1, or a salt thereof.
